(19) United States
(12) Patent Application Publication
(10) Pub. No.: US 2011/0092553 A1
(43) Pub. Date: Apr. 21, 2011

(54) Substituted Azole Derivatives, Compositions, and Methods of Use

(75) Inventors: Adnan M.M. Mjalli, Oak Ridge, NC (US); Dharma R. Polisetti, High Point, NC (US); Govindan Subramanian, Belle Mead, NJ (US); James C. Quada, JR., High Point, NC (US); Murty N. Arimilli, Oak Ridge, NC (US); Ravindra R. Yarragunta, Greensboro, NC (US); Robert C. Andrews, Jamestown, NC (US); Rongyuan Xie, Greensboro, NC (US)

(73) Assignee: TransTech Pharma, Inc., High Point, NC (US)

(21) Appl. No.: 12/950,967

(22) Filed: Nov. 19, 2010

Related U.S. Application Data
(63) Continuation of application No. 11/056,498, filed on Feb. 11, 2005, now abandoned.
(60) Provisional application No. 60/543,971, filed on Feb. 12, 2004.

(51) Int. Cl. A61K 31/433; C07D 233/64; C07D 403/10; C07D 285/06; A61K 31/4164; A61K 31/4178; A61K 31/417; A61P 3/10; A61P 35/00; A61P 3/04; A61P 37/00

(52) U.S. Cl. 514/361; 548/341.5; 548/338.1; 548/314.7; 548/311.4; 548/127; 514/399; 514/398; 514/397

(57) ABSTRACT

The present invention provides azole derivatives of Formula (I), methods of their preparation, pharmaceutical compositions comprising the compounds of Formula (I), and their use in treating human or animal disorders. The compounds of the invention can be useful as inhibitors of protein tyrosine phosphatases and thus can be useful for the management, treatment, control, or the adjunct treatment of diseases mediated by PTPase activity. Such diseases include Type I diabetes and Type II diabetes.

### STATEMENT OF RELATED APPLICATION

This application is a continuation of U.S. patent application Ser. No. 11/056,498, filed Feb. 11, 2005, which in turn claims priority to U.S. Provisional Patent Application No. 60/543,971, filed Feb. 12, 2004. The disclosures of each of the aforementioned applications are expressly incorporated by reference herein in their entirety as though fully set forth in the present application.

### FIELD OF THE INVENTION

This invention relates to substituted azole derivatives, compositions, and methods of treatment using the compounds and compositions which may be useful for the management, treatment, control, or adjunct treatment of diseases caused by activity of protein tyrosine phosphatases (PTPases).

### BACKGROUND OF THE INVENTION

Protein phosphorylation is now recognized as central to the fundamental processes of cellular signal transduction. Alterations in protein phosphorylation may therefore constitute either a physiological or pathological change in an in vivo system. Protein de-phosphorylation, mediated by phosphatases, is also central to certain signal transduction processes.

The two major classes of phosphatases are (a) protein serine/threonine phosphatases (PSTPases), which catalyze the dephosphorylation of serine and/or threonine residues on proteins or peptides; and (b) the protein tyrosine phosphatases (PTPases), which catalyze the dephosphorylation of tyrosine residues on proteins and/or peptides. A third class of phosphatases is the dual specificity phosphatases, or DSP's, which possess the ability to act both as PTPases and as PSTPases.

Among the PTPases there exist two important families, the intracellular PTPases, and the transmembrane PTPases. The intracellular PTPases include PTP1B, STEP, PTPD1, PTPD2, PTPMEG1, T-cell PTPase, PTPH1, FAP-1/BAS, PTP1D, and PTP1C. The transmembrane PTPases include LAR, CD45, PTPα, PTPβ, PTPδ, PTPε, PTPζ, PTPκ, PTPμ, PTPσ, HePTP, SAP-1, and PTP-U2. The dual—specificity phosphatases include KAP, cdc25, MAPK phosphatase, PAC-1, and rVH6.

The PTPases, especially PTP1B, are implicated in insulin insensitivity characteristic of type II diabetes (Kennedy, B. P.; Ramachandran, C. Biochem. Pharm. 2000, 60, 877-883). The PTPases, notably CD45 and HePTP, are also implicated in immune system function, and in particular T-cell function. Certain PTPases, notably TC-PTP, DEP-1, SAP-1, and CDC25, are also implicated in certain cancers. Certain PTPases, notably the bone PTPase OST-PTP, are implicated in osteoporosis. PTPases are implicated in mediating the actions of somatostatin on target cells, in particular the secretion of hormone and/or growth factor secretion.

Thus, there is a need for agents which inhibit the action of protein tyrosine phosphatases. Such agents would be useful for the treatment of Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### SUMMARY OF THE INVENTION

This invention provides substituted azole derivatives and compositions which inhibit PTP1B. In an embodiment, the present invention provides compounds of Formula (I) as depicted below. In another embodiment, the present invention provides methods of preparation of compounds of Formula (I). In another embodiment, the present invention provides pharmaceutical compositions comprising the compounds of Formula (I). In another embodiment, the present invention provides methods of using the compounds of Formula (I) in treating human or animal disorders. The compounds of the invention are useful as inhibitors of protein tyrosine phosphatases and thus may be useful for the management, treatment, control and adjunct treatment of diseases mediated by PTPase activity. Such diseases may comprise Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### DETAILED DESCRIPTION

Embodiments of the present invention comprise substituted azole derivatives, compositions, and methods of use. The present invention may be embodied in a variety of ways.

In a first aspect, the present invention provides azole inhibitors of protein tyrosine phosphatases (PTPases) which are useful for the management and treatment of disease caused by PTPases.

In a second aspect, the present invention provides compounds of Formula (I):

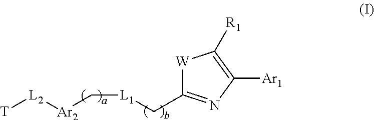

wherein a and b are, independently, equal to 0, 1, or 2, wherein the values of 0, 1, and 2 represent a direct bond, —CH₂—, and —CH₂CH₂—, respectively, and wherein the —CH₂— and —CH₂CH₂— groups are optionally substituted 1 to 2 times with a substituent group, comprising: -alkyl, -aryl, -alkylene-aryl, -arylene-alkyl, -alkylene-arylene-alkyl, —O-alkyl, —O-aryl, or -hydroxyl. In an embodiment, a and b are equal to 0.
W comprises —O—, —S—, or —N(R₂)—,

wherein
R₂ comprises
   a) -alkyl;
   b) -L₃-D₁-G₁-G₂;
   c) -L₃-D₁-alkyl:
   d) -L₃-D₁-aryl;
   e) -L₃-D₁-heteroaryl;
   f) -L₃-D₁-cycloalkyl;
   g) -L₃-D₁-heterocyclyl;
   h) -L₃-D₁-arylene-alkyl;
   i) -L₃-D₁-alkylene-arylene-alkyl;
   j) -L₃-D₁-alkylene-aryl;
   k) -L₃-D₁-alkylene-G₁-G₂;
   l) -L₃-D₁-arylene-G₁-G₂;
   m) -L₃-D₁-heteroarylene-G₁-G₂;
   n) -L₃-D₁-cycloalkylene-G₁-G₂;
   o) -L₃-D₁-heterocyclylene-G₁-G₂;
   p) -L₃-D₁-arylene-alkylene-G₁-G₂;
   q) -L₃-D₁-alkylene-arylene-alkylene-G₁-G₂;
   r) -L₃-D₁-alkylene-arylene-G₁-G₂;
   s) -L₃-D₁-arylene-D₂-G₁-G₂; and
   t) -L₃-D₁-alkylene-arylene-heteroarylene;
wherein
L₃ comprises a direct bond, -alkylene, -alkenylene, or alkynylene;
D₁ and D₂ independently comprise a direct bond, —CH₂—, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, —N(R₅)SO₂N(R₆)—, —N═N—, or —N(R₅)—N(R₆)—;
   wherein
      R₅ and R₆ independently comprise: -hydrogen, -alkyl, -aryl, -arylene-alkyl, -alkylene-aryl, or -alkylene-arylene-alkyl; and
         G₁ comprises a direct bond, -alkylene, -alkenylene, or alkynylene;
         G₂ comprises hydrogen, —CN, —SO₃H, —P(O)(OH)₂, —P(O)(O-alkyl)(OH), —CO₂H, —CO₂-alkyl, an acid isostere, —NR₇R₈, or

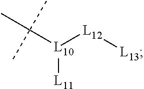

wherein
L₁₀ comprises alkyline, cycloalkyline, heteroaryline, aryline, or heterocyclyline;
L₁₂ comprises —O—, —C(O)—N(R₄₀)—, —C(O)—O—, —C(O)—, or —N(R₄₀)—CO—N(R₄₁)—;
L₁₃ comprises hydrogen, alkyl, alkenyl, alkynyl, heterocyclyl, heteroaryl, or -alkylene-aryl;
L₁₁ comprises hydrogen, alkyl, alkenyl, alkynyl, -alkylene-aryl, -alkylene -heteroaryl, alkylene-O-alkylene-aryl, -alkylene-S-alkylene-aryl, -alkylene-O-alkyl, -alkylene-S-alkyl, -alkylene-NH₂, -alkylene-OH, -alkylene-SH, -alkylene-C(O)—OR₄₂, -alkylene-C(O)—NR₄₂R₄₃, -alkylene-NR₄₂R₄₃, -alkylene-N(R₄₂)—C(O)—R₄₃, -alkylene-N(R₄₂)—S(O₂)—R₄₃, or the side chain of a natural or non-natural amino acid;
   wherein
      R₄₂ and R₄₃ independently comprise hydrogen, aryl, alkyl, or alkylene-aryl; or
         R₄₂ and R₄₃ may be taken together to form a ring having the formula —(CH₂)_{q}—Y—(CH₂)ₙ— bonded to the nitrogen atom to which R₄₂ and R₄₃ are attached, wherein q and r are, independently, 1, 2, 3, or 4; Y is —CH₂—, —C(O)—, —O—, —N(H)—, —S—, —S(O)—, —SO₂—, —CON(H)—, —NHC(O)—, —NHCON(H)—, —NHSO₂—, —SO₂N(H)—, —(O)CO—, —NHSO₂NH—, —OC(O)—, —N(R₄₄)—, —N(C(O)R₄₄)—, —N(C(O)NHR₄₄)—, —N(SO₂NHR₄₄)—, —N(SO₂R₄₄)—, and —N(C(O)OR₄₄)—; or
         R₄₂ and R₄₃ may be taken together, with the nitrogen atom to which they are attached, to form a heterocyclyl or heteroaryl ring; and
      R₄₀, R₄₁, and R₄₄ independently comprise hydrogen, aryl, alkyl, or alkylene-aryl.

and wherein
R₇ and R₈ independently comprise hydrogen, -alkyl, -L₄-E-alkyl, -L₄-E-aryl, —C(O)-alkyl, —C(O)-aryl, —SO₂-alkyl, —SO₂-aryl, or

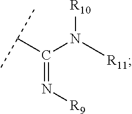

wherein
   R₉, R₁₀, and R₁₁ independently comprise: -hydrogen, -alkyl, -aryl, -arylene-alkyl, -alkylene-aryl, or -alkylene-arylene-alkyl;
   L₄ comprises a direct bond, -alkylene, -alkenylene, or -alkynylene;
   E comprises a direct bond, —CH₂—, —O—, —N(R₁₂)—, —C(O)—, —CON(R₁₂)—, —N(R₁₂)C(O)—, —N(R₁₂)CON(R₁₃)—, —N(R₁₂)C(O)O—, —OC(O)N(R₁₂)—, —N(R₁₂)SO₂—, —SO₂N(R₁₂)—, —C(O)—O—, —O—C(O)—, —S—, —S(O)—, —S(O₂)—, —N(R₁₂)SO₂N(R₁₃)—, —N═N—, or —N(R₁₂)—N(R₁₃)—,
      wherein
      R₁₂ and R₁₃ independently comprise: -hydrogen, -alkyl, -aryl, -arylene-alkyl, -alkylene-aryl, or -alkylene-arylene-alkyl.

In an embodiment, W comprises —N(R₂)—. In another embodiment, W comprises —N(R₂)—, wherein R₂ comprises alkyl, or -L₃-D₁-alkylene-aryl, wherein L₃ comprises alkylene, D₁ comprises —CO(NR₅)—, wherein R₅ comprises hydrogen. In another embodiment, W comprises —N(R₂)—, wherein R₂ comprises alkyl.

In another embodiment, W comprises —N(R₂)—, wherein R₂ comprises -L₃-D₁-arylene-D₂-G₁-G₂, wherein L₃ comprises a direct bond or alkylene, D₁ is a direct bond, D₂ is a direct bond, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, wherein R₅ and R₆ independently comprise: -hydrogen, -alkyl, -aryl, or -alkylene-aryl, G₁ is a direct bond or alkylene, and G₂ comprises —CO₂H, —CO₂-alkyl, or an acid isostere, wherein the arylene group may be optionally substituted with halo, —O-alkyl optionally substituted 1 to 5 times with halo, and -alkyl optionally substituted 1 to 5 times with halo. In another embodiment, W comprises —N(R₂)—, wherein R₂ comprises a phenyl group or benzyl group wherein the benzene ring is substituted with a group selected from the group consisting of —CO₂H, —CO₂-alkyl, -acid isostere, —NHCH₂CO₂H, and —N(SO₂CH₃)CH₂CO₂H, and further optionally substituted with a group selected from the group consisting of -halo, -perhaloalkyl, and —NHSO₂CH₃. In another embodiment, W comprises —N(R₂)—, wherein R₂ comprises -methylene-benzoic acid.

R₁ comprises

a) -hydrogen;

b) -fluoro;

c) -chloro;

d) -bromo;

e) -iodo;

f) -cyano;

g) -alkyl;

h) -aryl;

i) -alkylene-aryl;

j) -heteroaryl;

k) -alkylkene-heteroaryl;

l) -cycloalkyl;

m) -alkylene-cycloalkyl

n) -heterocyclyl; or

o) -alkylene-heterocyclyl;

In another embodiment, R₁ comprises hydrogen or aryl. In another embodiment, R₁ comprises hydrogen.

L₁ comprises

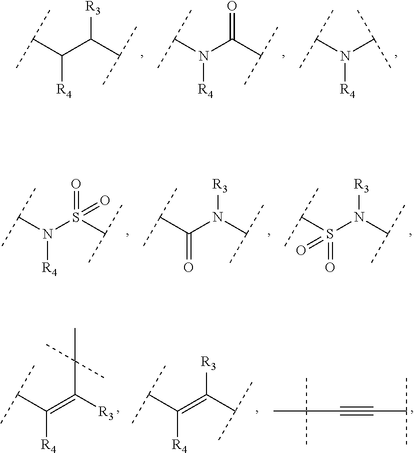

—C(O)—, -alkylene-O—, —CH₂—, 1,1 cycloalkylmethylene, or a direct bond

In an embodiment, L₁ comprises

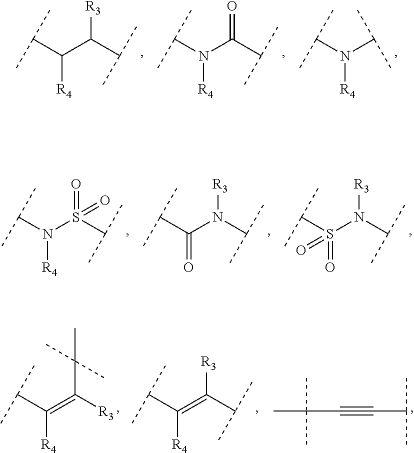

or a direct bond;
wherein R₃ and R₄ independently comprise hydrogen, chloro, fluoro, bromo, alkyl, aryl, -alkylene-aryl, -cycloalkyl, -alkylene-cycloalkyl, -heterocyclyl, -alkylene-heterocyclyl, or -alkynylene. In another embodiment, L₁ comprises

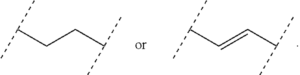

In another embodiment, L₁ comprises

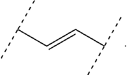

In another embodiment, L₁ comprises —CH₂—, or —CH₂—O—.

Ar₁ comprises an aryl, heteroaryl, fused cycloalkylaryl, fused cycloalkylheteroaryl, fused heterocyclylaryl, or fused heterocyclylheteroaryl group optionally substituted 1 to 7 times. In an embodiment, Ar₁ comprises a mono- or bicyclic aryl group optionally substituted 1 to 7 times. In another embodiment, Ar₁ comprises a phenyl or naphthyl group optionally having 1 to 5 substituents. In an embodiment, the substituents independently comprise:

a) -fluoro;

b) -chloro;

c) -bromo;

d) -iodo;

e) -cyano;

f) -nitro;

g) -perfluoroalkyl;

h) -J-R₁₄;

i) -alkyl;

j) -aryl;

k) -heteroaryl;

l) -heterocyclyl;

m) -cycloalkyl;

n) -L₅-aryl;

o) -L₅-arylene-aryl;

p) -L₅-arylene-alkyl;

q) -arylene-alkyl;

r) -arylene-arylene-alkyl;

s) -J-alkyl;

t) -J-aryl;

u) -J-alkylene-aryl;

v) -J-arylene-alkyl;

w) -J-alkylene-arylene-aryl;

x) -J-arylene-arylene-aryl;

y) -J-alkylene-arylene-alkyl;

z) -L₅-J-alkylene-aryl;

aa) -arylene-J-alkyl;

bb) -L₅-J-aryl;

cc) -L₅-J-heteroaryl;

dd) -L₅-J-cycloalkyl;

ee) -L₅-J-heterocyclyl;

ff) -L₅-J-arylene-alkyl;

gg) -L₅-J-alkylene-arylene-alkyl;

hh) -L₅-J-alkyl;

ii) -L₅-J-R₁₄;

jj) -arylene-J-R₁₄; or

kk) -hydrogen;

wherein L₅ comprises a direct bond, -alkylene, -alkenylene, or -alkynylene; and wherein J comprises a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—, and wherein R₁₄, R₁₅, and R₁₆ independently comprise: -hydrogen, -alkyl, -aryl, -arylene-alkyl, -alkylene-aryl, or -alkylene-arylene-alkyl.

In another embodiment, Ar₁ comprises a phenyl group substituted 1 to 5 times, wherein the substituents independently comprise:

a) -fluoro;

b) -chloro;

c) -bromo;

d) -iodo;

e) -cyano;

f) -nitro; or

g) -aryl.

In another embodiment, Ar₁ comprises a phenyl group substituted 1 to 5 times, wherein the substituents comprise: -chloro or -fluoro.

Ar₂ comprises an arylene, heteroarylene, fused arylcycloalkylene, fused cycloalkylarylene, fused cycloalkylheteroarylene, fused heterocyclylarylene, or fused heterocyclylheteroarylene group optionally substituted 1 to 7 times. Ar₂ may also be taken in combination with R₄ to constitute a fused arylcycloalkylene, fused cycloalkylarylene, fused cycloalkylheteroarylene, fused heterocyclylarylene, or fused heterocyclylheteroarylene group, optionally substituted 1 to 7 times. In an embodiment, Ar₂ comprises a arylene group optionally substituted 1 to 7 times. In another embodiment, Ar₂ comprises a phenylene or naphthylene group optionally having 1 to 5 substituents. In an embodiment, the substituents independently comprise:
a) -fluoro;
b) -chloro;
c) -bromo;
d) -iodo;
e) -cyano;
f) -nitro;
g) -perfluoroalkyl;
h) -Q-R₁₇;
i) -alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s) -Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y) -Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L₆-Q-arylene-alkyl;
gg) -L₆-Q-alkylene-arylene-alkyl;
hh) -L₆-Q-alkyl;
ii) -L₆-Q-alkylene-aryl-R₁₇;
jj) -L₆-Q-alkylene-heteroaryl-R₁₇;
kk) -arylene-Q-alkylene-R₁₇;
ll) -heteroarylene-Q-alkylene-R₁₇;
mm) -L₆-Q-aryl-R₁₇;
nn) -L₆-Q-heteroarylene-R₁₇;
oo) -L₆-Q-heteroaryl-R₁₇;
pp) -L₆-Q-cycloalkyl-R₁₇;
qq) -L₆-Q-heterocyclyl-R₁₇;
rr) -L₆-Q-arylene-alkyl-R₁₇;
ss) -L₆-Q-heteroarylene-alkyl-R₁₇;
tt) -L₆-Q-alkylene-arylene-alkyl-R₁₇;
uu) -L₆-Q-alkylene-heteroarylene-alkyl-R₁₇;
vv) -L₆-Q-alkylene-cycloalkylene-alkyl-R₁₇;
ww) -L₆-Q-alkylene-heterocyclylene-alkyl-R₁₇;
xx) -L₆-Q-alkyl-R₁₇;
yy) -L₆-Q-R₁₇;
zz) -arylene-Q-R₁₇;
aaa) -heteroarylene-Q-R₁₇;
bbb) -heterocyclylene-Q-R₁₇;
ccc) -Q-alkylene-R₁₇;
ddd) -Q-arylene-R₁₇;
eee) -Q-heteroarylene-R₁₇;
fff) -Q-alkylene-arylene-R₁₇;
ggg) -Q-alkylene-heteroarylene-R₁₇;
hhh) -Q-heteroarylene-alkylene-R₁₇;
iii) -Q-arylene-alkylene-R₁₇;
jjj) -Q-cycloalkylene-alkylene-R₁₇;
kkk) -Q-heterocyclylene-alkylene-R₁₇
lll) -Q-alkylene-arylene-alkyl-R₁₇;
mmm) -Q-alkylene-heteroarylene-alkyl-R₁₇;

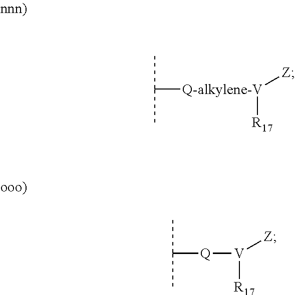

or
ppp) -hydrogen

wherein
L₆ comprises a direct bond, -alkylene, -alkenylene, or -alkynylene;
Q comprises a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₈)SO₂N(R₁₉)—, —N═N—, or —N(R₁₈)—N(R₁₉)—;
   wherein
      R₁₈ and R₁₉ independently comprise: -hydrogen, -alkyl, -aryl, -arylene-alkyl, -alkylene-aryl, or -alkylene-arylene-alkyl;
V comprises

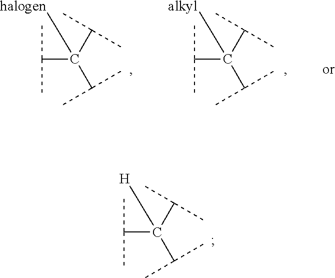

Z comprises hydrogen, -alkylene-aryl, -alkyl, -aryl, -heteroaryl, -heterocyclyl, -cycloalkyl, -alkylene-heteroaryl, or -alkylene-cycloalkyl;
R₁₇ comprises —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, acyloxy-alkylene-, or -alkylene-arylene-alkyl.

In another embodiment, Ar₂ comprises a phenyl group or naphthyl group substituted 1 to 5 times, wherein the substituents independently comprise:

a) -fluoro;

b) -chloro;

c) -bromo;

d) -iodo;

h) -Q-R₁₇;

i) -alkyl;

j) -aryl;

q) -arylene-alkyl;

s) -Q-alkyl; or

t) -arylene-Q-alkyl;

wherein
Q comprises —CH₂—, —O—, —C(O)—, —C(O)—O—, and
R₁₇ comprises: -hydrogen, -alkyl, -aryl, —CO₂H, or an acid isostere.

In another embodiment, Ar₂ comprises a phenyl group substituted 1 to 5 times, wherein the substituents independently comprise:

a) -fluoro;

b) -chloro;

c) -bromo;

d) -iodo;

e) -Q-R₁₇;

f) -alkyl;

g) -phenyl;

h) -phenylene-alkyl;

i) -Q-alkyl; or

j) -phenylene-Q-alkyl;

wherein
Q comprises —CH₂—, —O—, —C(O)—, —C(O)—O—, and
R₁₇ comprises: -hydrogen, -alkyl, -phenyl, or —CO₂H.

L₂ comprises: —CH₂—, —O—, —K—, -alkylene-, -alkenylene-, -alkynelene-, —K-alkylene-, -alkylene-K—, -alkylene-K-alkylene-, -alkenylene-K-alkylene-, -alkylene-K-alkenylene-, -arylene-K-alkylene-, alkylene-K-arylene-, -heteroarylene-K-alkylene-, alkylene-K-heteroarylene-, -arylene-K—, —K-arylene-, -heteroarylene-K—, —K-heteroarylene-, or a direct bond,
wherein K comprises a direct bond, —O—, —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂—, —SO₂N(R₂₀)—, —C(O)—O—, —O—C(O)—, —S—, —S(O)—, —S(O₂)—, —N(R₂₀)SO₂N(R₂₁)—, —N═N—, or —N(R₂₀)—N(R₂₁)—; wherein R₂₀ and R₂₁ independently comprise: -hydrogen, -alkyl, -aryl, -arylene-alkyl, -alkylene-aryl, or -alkylene-arylene-alkyl.

In an embodiment, L₂ comprises: —O—, —O-alkylene-, -alkylene-O, or a direct bond. In another embodiment, L₂ comprises: —O-alkylene- or a direct bond. In another embodiment, L₂ comprises —K—, wherein K comprises —O—, —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂⁻, —SO₂N(R₂₀)—, —C(O)—O—, —O—C(O)—, —S—, —S(O)—, —S(O₂)—, —N(R₂₀)SO₂N(R₂₁)—, —N═N—, or —N(R₂₀)—N(R₂₁)—. In another embodiment, L₂ comprises —K—, wherein K comprises —N(R₂₀)CO—, wherein R₂₀ comprises hydrogen or alkyl.

T comprises: hydrogen, alkyl, cycloalkyl, heterocyclyl, aryl, heteroaryl, fused cycloalkylaryl, fused cycloalkylheteroaryl, fused heterocyclylaryl, or fused heterocyclylheteroaryl group optionally substituted 1 to 7 times. In an embodiment, T comprises an alkyl, -alkylene-aryl, or aryl group optionally substituted 1 to 7 times. In another embodiment, T comprises an aryl group optionally having 1 to 5 substituents. In an embodiment, the substituents independently comprise:

a) -fluoro;

b) -chloro;

c) -bromo;

d) -iodo;

e) -cyano;

f) -nitro;

g) -perfluoroalkyl;

h) —U₁-perfluoroalkyl;

i) —U₁—R₂₂;

j) -alkyl;

k) -aryl;

l) -heteroaryl;

m) -heterocyclyl;

n) -cycloalkyl;

o) -L₇-aryl;

p) -L₇-arylene-aryl;

q) -L₇-arylene-alkyl;

r) -arylene-alkyl;

s) -arylene-arylene-alkyl;

t) —U₁-alkyl;

u) —U₁-aryl;

v) —U₁-alkylene-aryl;

w) —U₁-arylene-alkyl;

x) —U₁-alkylene-arylene-aryl;

y) —U₁-arylene-arylene-aryl;

z) —U₁-alkylene-arylene-alkyl;

aa) -L₇-U₁-alkylene-aryl;

bb) -arylene-U₁-alkyl;

cc) -L₇-U₁-aryl;

dd) -L₇-U₁-heteroaryl;

ee) -L₇-U₁-cycloalkyl;

ff) -L₇-U₁-heterocyclyl;

gg) -L₇-U₁-arylene-alkyl;

hh) -L₇-U₁-alkylene-arylene-alkyl;

ii) -L₇-U₁-alkyl;

jj) -L₇-U₁-alkylene-aryl-R₂₂;

kk) -L₇-U₁-alkylene-heteroaryl-R₂₂;

ll) -arylene-U₁-alkylene-R₂₂;

mm) -heteroarylene-U₁-alkylene-R₂₂;

nn) -L₇-U₁-aryl-R₂₂;

oo) -L₇-U₁-heteroarylene-R₂₂;

PP) -L₇-U₁-heteroaryl-R₂₂;

qq) -L₇-U₁cycloalkyl-R₂₂;

rr) -L₇-U₁-heterocyclyl-R₂₂;

ss) -L₇-U₁-arylene-alkyl-R₂₂;

tt) -L₇-U₁-heteroarylene-alkyl-R₂₂;

uu) -L₇-U₁-alkylene-arylene-alkyl-R₂₂;

vv) -L₇-U₁-alkylene-heteroarylene-alkyl-R₂₂;

ww) -L₇-U₁-alkylene-cycloalkylene-alkyl-R₂₂;

xx) -L₇-U₁-alkylene-heterocyclylene-alkyl-R₂₂;

yy) -L₇-U₁-alkylene-R₂₂;

zz) -L₇-U₁—R₂₂;

aaa) -arylene-U₁—R₂₂;

bbb) -heteroarylene-U₁—R₂₂;

ccc) -heterocyclylene-U₁—R₂₂;

ddd) —U₁-alkylene-R₂₂;

eee) —U₁arylene-R₂₂;

fff) —U₁-heteroarylene-R₂₂;

ggg) —U₁-alkylene-arylene-R₂₂;

hhh) —U₁-alkylene-heteroarylene-R₂₂;

iii) —U₁-heteroarylene-alkylene-R₂₂;

jjj) —U₁-arylene-alkylene-R₂₂;

kkk) —U₁-cycloalkylene-alkylene-R₂₂;

lll) —U₁-heterocyclylene-alkylene-R₂₂;

mmm) —U₁-alkylene-arylene-alkyl-R₂₂;

nnn) —U₁-alkylene-heteroarylene-alkyl-R₂₂;

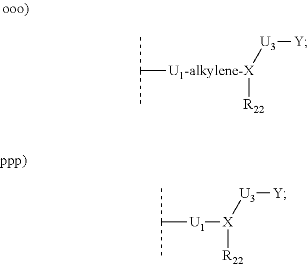

qqq) —U₁-alkylene-U₂-alkyl;

rrr) —U₁—U₂-alkyl; or

sss) -hydrogen
wherein
   L₇ comprises a direct bond, -alkylene, -alkenylene, or -alkynylene;
   U₁, U₂, and U₃ independently comprise a direct bond, —CH₂—, —O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;
      wherein
         R₂₃ and R₂₄ independently comprise: -hydrogen, —U₅-alkyl, —U₅-aryl, —U₅-perhaloalkyl, -arylene-alkyl, -alkylene-aryl, or -alkylene-arylene-alkyl; wherein U₅ comprises a direct bond, —SO₂—, —CO—, or —SO₂—NHCO₂—;
         or wherein T comprises

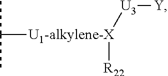

R₂₃ or R₂₄ may be fused with the alkylene group between U₁ and X to form a 5 to 7 membered ring;
X comprises

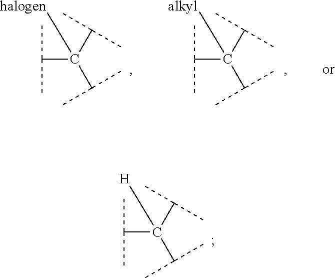

Y comprises hydrogen, -alkylene-aryl, -alkyl, -aryl, -heteroaryl, -heterocyclyl, -cycloalkyl, -alkylene-heteroaryl, or -alkylene-cycloalkyl;
R₂₂ comprises —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, acyloxy-alkylene-, or -alkylene-arylene-alkyl.

In another embodiment, T comprises an aryl group substituted by —U₁-alkylene-R₂₂, wherein U₁ comprises —O— or a direct bond, and R₂₂ comprises —CO₂H or an acid isostere.

In another embodiment, —Ar₂-L₂-T together comprise a biphenyl group substituted with at least one group selected from the group consisting of

—U₁-alkyl,

—U₁-perhaloalkyl,

—U₁—R₂₂,

fluoro, and

chloro,
wherein
   U₁ comprises a direct bond, —CO₂—, —O—, —S—, —NHSO₂—, —N(R₂₃)SO₂—, —CONH—SO₂—, —SO₂—, —NHCO—, —NHCO₂—, —NHCO₂NH—, wherein R₂₃ comprises —U₅-alkyl, wherein U₅ comprises a direct bond or —SO₂—,
   R₂₂ comprises alkyl, —CO₂H or acid isostere, and
wherein
   the alkyl group may be optionally substituted 1 to 5 times with halo.

In another embodiment, —Ar₂-L₂-T together comprise phenoxy-biphenylene group, wherein the phenyoxy group is substituted with at least one group selected from the group consisting of

—U₁-alkyl,

—U₁-perfluoroalkyl, and

—U₁—R₂₂,
wherein
   U₁ comprises a direct bond, —CO₂—, —O—, —S—, —NHSO₂—, —N(R₂₅)SO₂—, —CONH—SO₂—, —SO₂—, —NHCO—, —NHCO₂—, —NHCO₂NH—, wherein R₂₃ comprises —U₅-alkyl, wherein U₅ comprises a direct bond or —SO₂—,
   R₂₂ comprises alkyl, —CO₂H or acid isostere, and
wherein
   the alkyl group may be optionally substituted 1 to 5 times with halo.

In another embodiment of the compound of Formula (I), Ar₁ comprises: 2,4-dichlorophenyl.

In another embodiment of the compound of Formula (I), W comprises —N(R₂)—, wherein R₂ comprises -L₃-D₁-arylene-G₁-G₂, wherein
L₃ comprises alkylene,
D₁ is a direct bond,
G₁ is a direct bond or alkylene, and
G₂ comprises —CN, —SO₃H, —P(O)(OH)₂, —P(O)(O-alkyl)(OH), —CO₂H, —CO₂-alkyl, or an acid isostere.

In another embodiment of the compound of Formula (I), W comprises —N(R₂)—, wherein R₂ comprises -L₃-D₁-alkylene-arylene-G₁-G₂, wherein
L₃ comprises alkylene,
D₁ comprises —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, —N═N—, or —N(R₅)—N(R₆)—, wherein R₅ and R₆ are -hydrogen;
G₁ comprises a direct bond or alkylene; and
G₂ comprises —CN, —SO₃H, —P(O)(OH)₂, —P(O)(O-alkyl)(OH), —CO₂H, —CO₂-alkyl, or an acid isostere.

In another embodiment of the compound of Formula (I),

Ar₂ comprises phenyl,
L₂ comprises a direct bond, —K— or -arylene-K—; wherein K comprises —NH₂—OH₂—, —NH₂—SO₂—, —N(alkyl)-SO₂—, or —O—
T comprises phenyl substituted with at least one group comprising

a) -fluoro;

b) -chloro;

c) -cyano;

d) -perfluoroalkyl;

e) —U₁-perfluoroalkyl;

f) —U₁-alkylene-R₂₂;

g) —U₁—R₂₂; or

e) -alkyl substituted 1 to 5 times with halo;

wherein
U₁ comprises —O—, direct bond, —SO₂—, or —NHSO₂—; and
R₂₂ comprises -alkyl, —SO₃H, —P(O)(OH)₂, —P(O)(O-alkyl)(OH), —CO₂H, —CO₂-alkyl, or an acid isostere.

In another embodiment of the compound of Formula (I),

Ar₂ comprises phenyl,
L₂ comprises a direct bond,
T comprises thiophenyl substituted with at least one group comprising

a) -halo;

b)-alkyl;

c)-alkyl substituted 1 to 5 times with halo; or

d) —U₁—R₂₂;

wherein
U₁ comprises —O—, direct bond, —SO₂—, or —NHSO₂—; and
R₂₂ comprises -alkyl, —SO₃H, —P(O)(OH)₂, —P(O)(O-alkyl)(OH), —CO₂H, —CO₂-alkyl, or an acid isostere.

In another embodiment of the compound of Formula (I), wherein a and b are equal to zero, -L₁-Ar₂-L₂-T together comprise a group selected from the group consisting of: 2-[alkyl-benzenesulfonylamino-phenyl]-(E)-vinyl, 2-[(alkyl-benzylamino)-phenyl]-(E)-vinyl, 2-[(trifluoroalkyl-benzenesulfonylamino)-phenyl]-(E)-vinyl, 2-{[(alkyl-benzenesulfonyl)-alkyl-amino]-phenyl}-(E)-vinyl, 2-(4′-trifluoroalkoxy-biphenyl-4-yl)-(E)-vinyl, 2-(3′-trifluoroalkylsulfonyl amino-biphenyl-4-yl)-(E)-vinyl, 2-(3′-carboxy-biphenyl-4-yl)-(E)-vinyl, 2-(4′-carboxy-biphenyl-4-yl)-(E)-vinyl, 2-(3′-alkylsulfonyl-biphenyl-4-yl)-(E)-vinyl, 2-{4′-[(trifluoromethanesulfonamide)-phenyoxy]-biphenyl-4-yl}-(E)-vinyl, 2-{4′-[bis(trifluoromethanesulfonimide)-phenyoxy]-biphenyl-4-yl}-(E)-vinyl, 2-{4′-[(N-methyl-trifluoromethanesulfonamide)-phenyoxy]-biphenyl-4-yl}-(E)-vinyl, 2-[4′-(4-alkylsulfonylamino-phenoxy)-biphenyl-4-yl]-(E)-vinyl, 2-[4-(5-Chloro-thiophen-2-yl)-phenyl]-(E)-vinyl, 2-(4′-alkylsulfanyl-biphenyl-4-yl)-(E)-vinyl, 2-[(4-pyrimidin-3-yl)-phenyl]-(E)-vinyl, 2-[4-(5-acetyl-thiophen-2-yl-phenyl)]-(E)-vinyl, 2-[3′-(1,1,4-trioxo-1-[1,2,5]-thiadiazolidin-2-yl)-biphenyl-4-yl]-(E)-vinyl, 2-(4′-alkoxyoxycarbonylamino-3′-alkoxyoxy-biphenyl-4-yl)-(E)-vinyl, 2-(4′-amino-3′-alkoxy-biphenyl-4-yl)-(E)-vinyl, 2-[4′-(3-isopropyl-ureido)-3′-alkoxyoxy-biphenyl-4-yl]-(E)-vinyl, and 2-[4-(trifluoroalkyl-phenoxy)-phenyl]-(E)-vinyl.

In another embodiment of the compound of Formula (I), wherein a and b are equal to zero, -L₁-Ar₂-L₂-T together comprise a group selected from the group consisting of: 3′-trifluoroalkyl-biphenyl-4-ylmethyl, 4′-trifluoroalkyl-biphenyl-4-ylmethyl, (3′-alkylsulfonylamino-biphenyl-4-yl)-methyl, (4′-alkylsulfonylamino-biphenyl-4-yl)-methyl, [4′-(trifluoromethanesulfonylamino-carboxy)-phenyoxy]-biphenyl-4-ylmethyl, or 4′-[(trifluoromethyl-carboxy)-phenoxy]-biphenyl-4-yloxyethyl.

In another embodiment of the compound of Formula (I), wherein a and b are equal to zero, -L₁-Ar₂-L₂-T together comprise a group selected from the group consisting of: 4′-tert-butoxycarbonylamino-3′-methoxy-biphenyl-4-yl or 4′-alkylsulfonylamino-3′-alkoxyoxy-biphenyl-4-yl.

In another embodiment, the present invention provides the compound of Formula (I)

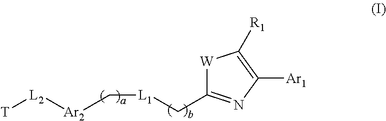

wherein W comprises —N—R₂, and wherein the compound of Formula (I) comprises one or more groups having at least a partial negative charge at physiological pH or a biohydrolyzable ester or biohydrolyzable amide thereof. In an embodiment, either T-L₂-Ar₂— together comprise a group having at least a partial negative charge at physiological pH or a prodrug thereof or R₂ comprises a group having at least a partial negative charge at physiological pH or a biohydrolyzable ester or biohydrolyzable amide thereof. Groups that may have at least a partial negative at physiological pH include, but are not limited to, —SO₃H, —P(O)(OH)₂, —P(O)(O-alkyl)(OH), —CO₂H, and an acid isostere.

The alkyl, aryl, heteroaryl, alkylene, arylene, and heteroarylene groups in Ar₁, Ar₂, and in R₁ through R₄₄ and Y may be optionally substituted 1 to 5 times with a substituent selected from the group consisting of:

a) -halogen;

b) -perhaloalkyl;

c) -hydroxyl;

d) —U₄-alkyl; and

e) —U₄-alkylene-aryl;

wherein U₄ is selected from the group consisting of —CH₂—, —O—, —N(H)—, —S—, —SO₂—, —CON(H)—, —NHC(O)—, —NHCON(H)—, —NHSO₂—, —SO₂N(H)—, —CO₂—, —NHSO₂NH—, and —O—CO—.

In the compounds of Formula (I), the various functional groups represented should be understood to have a point of attachment at the functional group having the hyphen. In other words, in the case of -alkylene-aryl, it should be understood that the point of attachment is the alkylene group; an example would be benzyl. In the case of a group such as —C(O)—NH— alkylene-aryl, the point of attachment is the carbonyl carbon.

Also included within the scope of the invention are the individual enantiomers of the compounds represented by Formula (I) above as well as any wholly or partially racemic mixtures thereof. The present invention also covers the individual enantiomers of the compounds represented by formula above as mixtures with diastereoisomers thereof in which one or more stereocenters are inverted.

In another aspect, the present invention provides a pharmaceutically acceptable salt, solvate, or prodrug of compounds of Formula (I). In an embodiment, the prodrug comprises a biohydrolyzable ester or biohydrolyzable amide of a compound of Formula I.

Examples of compounds of Formula (I) of the present invention having potentially useful biological activity are listed by name below in Table 1. The ability of compounds Formula (I) to inhibit PTP-1B was established with representative compounds of Formula (I) listed in Table I using a standard primary/secondary assay test procedure that measures the inhibition of PTP-1B activity. The compounds of Formula I in Table I may inhibit PTP-IB with an IC50 of less than 20 microMolar (μM; 10⁻⁶ M).

Compounds that inhibit PTP-1B activity are potentially useful in treating metabolic disorders related to insulin resistance or hyperglycemia, typically associated with obesity or glucose intolerance. The compounds of Formula (I) of the present invention may therefore be particularly useful in the treatment or inhibition of type II diabetes. The compounds of this invention may also potentially be useful in modulating glucose levels in disorders such as type I diabetes.

TABLE 1

| Ex. | Structure | Name |
|---|---|---|
| 1 | 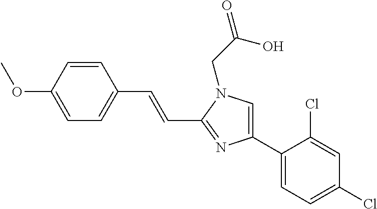 | {4-(2,4-dichloro-phenyl)-2- [2-(4-methoxy-phenyl)- (E)-vinyl]-imidazol-1-yl}- acetic acid |
| 2 | 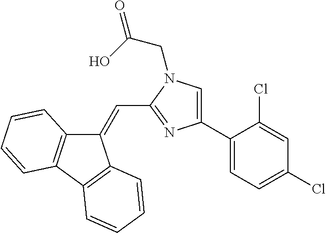 | [4-(2,4-dichloro-phenyl)-2- fluoren-9-ylidenemethyl- imidazol-1-yl]acetic acid |
| 3 | 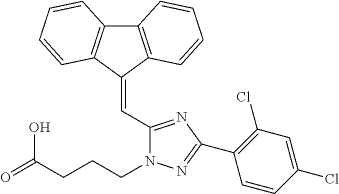 | 4-[4-(2,4-dichloro-phenyl)- 2-fluoren-9-ylidenemethyl- imidazol-1-yl]-butyric acid |
| 4 | 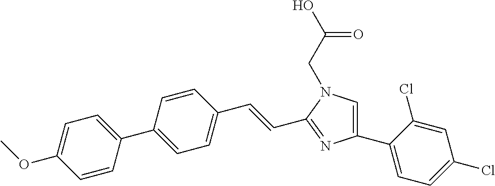 | {4-(2,4-dichloro-phenyl)-2- [2-(4′-methoxy-biphenyl- 4-yl)-(E)-vinyl]-imidazol-1- yl}-acetic acid |
| 5 | 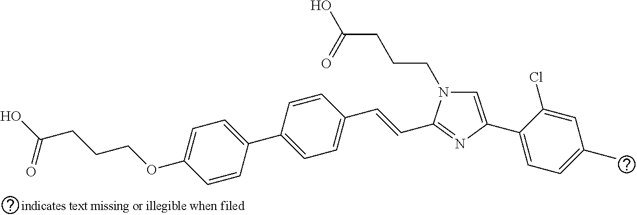 | 4-[2-{2-[4′-(3-carboxy- propoxy)-biphenyl-4-yl]- (E)-vinyl}-4-(2,4-dichloro- phenyl)-imidazol-1-yl]- butyric acid |
| 6 | 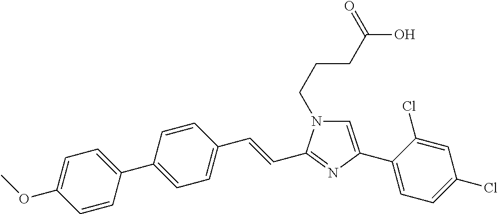 | 4-{4-(2,4-dichloro-phenyl)- 2-[2-(4′-methoxy- biphenyl-4-yl)-(E)-vinyl]- imidazol-1-yl}-butyric acid |
| 7 | 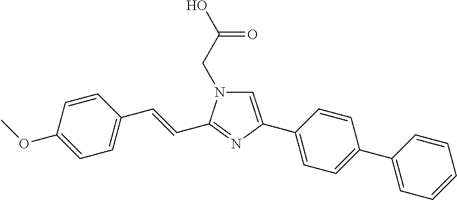 | {4-biphenyl-4-yl-2-[2-(4- methoxy-phenyl)-(E)- vinyl]-imidazole-1yl}- acetic acid |
| 8 | 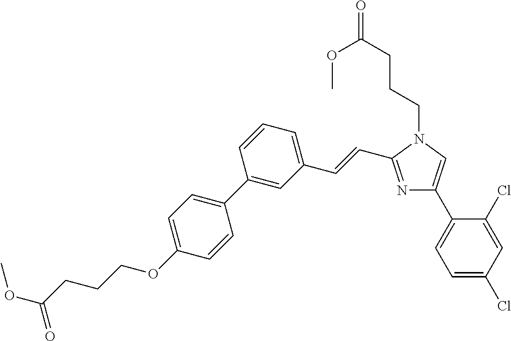 | 4-(4-(2,4-dichloro-phenyl)- 2-{2-[4′-(3- methoxycarbonyl- propoxy)-biphenyl-3yl]- (E)-vinyl}-imidazol-1yl)- butyric acid methyl ester |
| 9 | 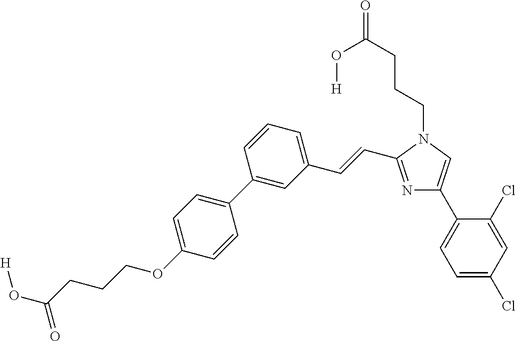 | 4-[2-{2-[4′-(3-carboxy- propoxy)-biphenyl-3-yl]- (E)-vinyl}-4-(2,4-dichloro- phenyl)-imidazol-1-yl]- butyric acid |
| 10 | 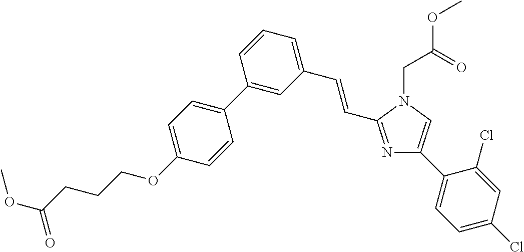 | 4-(3′-{2-[4-(2,4-dichloro- phenyl)-1- methoxycarbonylmethyl- 1H-imidazol-2-yl]-(E)- vinyl}-biphenyl-4yloxy)- butyric acid methyl ester |
| 11 | 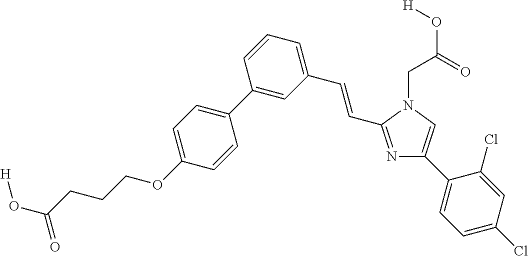 | 4-(3′-{2-[4-(2,4-dichloro- phenyl)-1- methoxycarbonylmethyl- 1H-imidazol-2-yl]-(E)- vinyl}-biphenyl-4yloxy)- butyric acid |
| 12 | 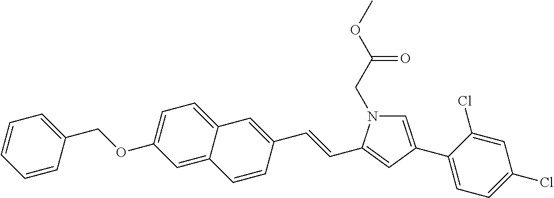 | 2-[2-(6-benzyloxy- naphthalen-2-yl)-(E)- vinyl]-4-(2,4-dichloro- phenyl)-imidazol-1-yl]- acetic acid methyl ester |
| 13 | 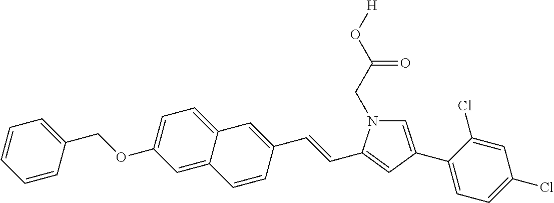 | 2-[2-(6-benzyloxy- naphthalen-2-yl)-(E)- vinyl]-4-(2,4-dichloro- phenyl)-imidazol-1-yl]- acetic acid methyl ester |
| 14 | 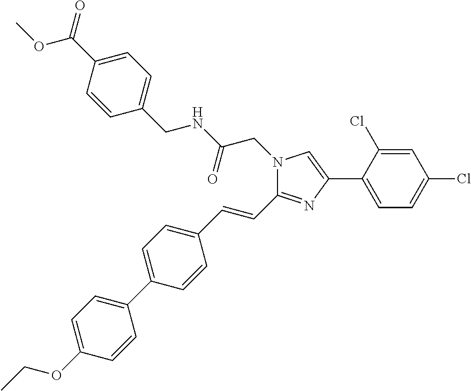 | 4-[(2-{4-(2,4-Dichloro- phenyl)-2-[2-(4′-ethoxy- biphenyl-4-yl)-(E)-vinyl]- imidazol-1-yl}- acetylamino)-methyl]- benzoic acid methyl ester |
| 15 | 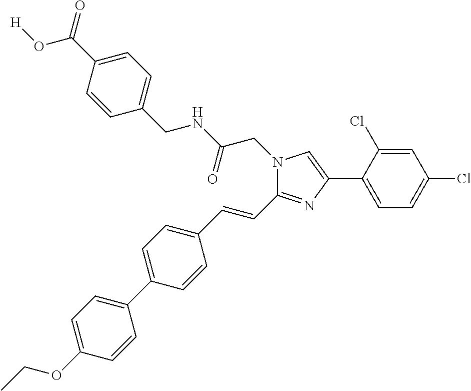 | 4-[(2-{4-(2,4-Dichloro- phenyl)-2-[2-(4′-ethoxy- biphenyl-4-yl)-(E)-vinyl]- imidazol-1-yl}- acetylamino)-methyl]- benzoic acid |
| 16 | 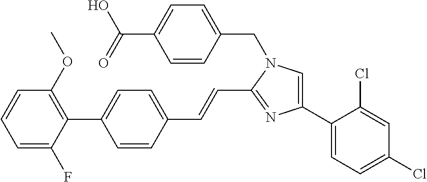 | 4-{4-(2,4-dichloro-phenyl)- 2-[2-(6′-fluoro-2′-methoxy- biphenyl-4-yl)-(E)-vinyl]- imidazol-1-ylmethyl}- benzoic acid |
| 17 | 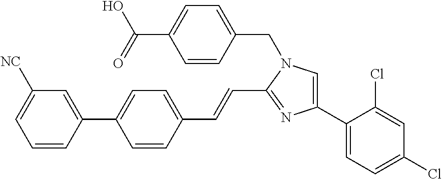 | 4-[2-[2-(3′-cyano- biphenyl-4-yl)-(E)-vinyl]-4- (2,4-dichloro-phenyl)- imidazol-1-ylmethyl]- benzoic acid |
| 18 | 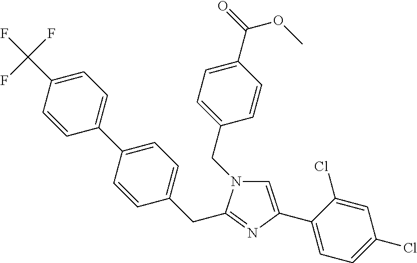 | 4-[4-(2,4-dichloro-phenyl)- 2-(4′-trifluoromethyl- biphenyl-4-ylmethyl)- imidazol-1-ylmethyl]- benzoic acid methyl ester |
| 19 | 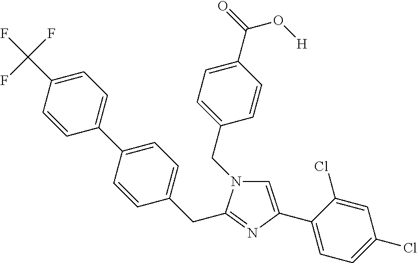 | 4-[4-(2,4-dichloro-phenyl)- 2-(4′-trifluoromethyl- biphenyl-4-ylmethyl)- imidazol-1-ylmethyl]- benzoic acid |
| 20 | 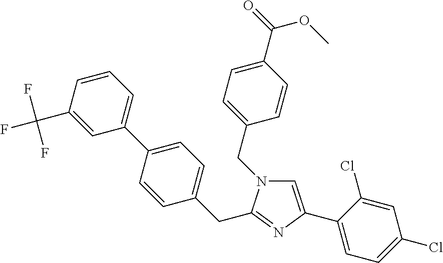 | 4-[4-(2,4-dichloro-phenyl)- 2-(3′-trifluoromethyl- biphenyl-4-ylmethyl)- imidazol-1-ylmethyl]- benzoic acid methyl ester |
| 21 | 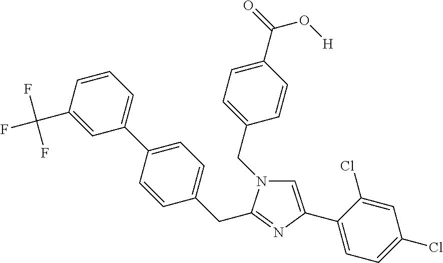 | 4-[4-(2,4-dichloro-phenyl)- 2-(3′-trifluoromethyl- biphenyl-4-ylmethyl)- imidazol-1-ylmethyl]- benzoic acid |
| 22 | 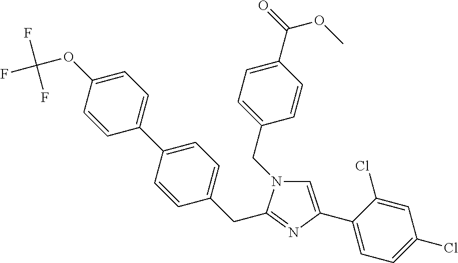 | 4-[4-(2,4-dichloro-phenyl)- 2-(4′-trifluoromethoxy- biphenyl-4-ylmethyl)- imidazol-1-ylmethyl]- benzoic acid methyl ester |
| 23 | 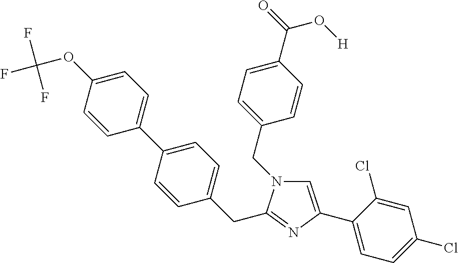 | 4-[4-(2,4-dichloro-phenyl)- 2-(4′-trifluoromethoxy- biphenyl-4-ylmethyl)- imidazol-1-ylmethyl]- benzoic acid |
| 24 | 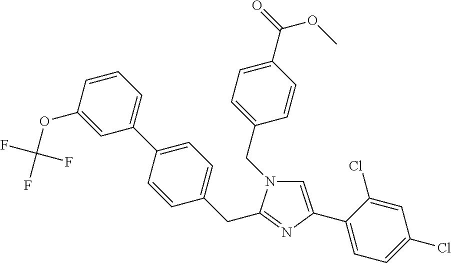 | 4-[4-(2,4-dichloro-phenyl)- 2-(3′-trifluoromethoxy- biphenyl-4-ylmethyl)- imidazol-1-ylmethyl]- benzoic acid methyl ester |
| 25 | 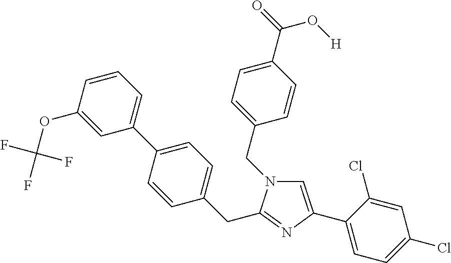 | 4-[4-(2,4-dichloro-phenyl)- 2-(3′-trifluoromethoxy- biphenyl-4-ylmethyl)- imidazol-1-ylmethyl]- benzoic acid |
| 26 | 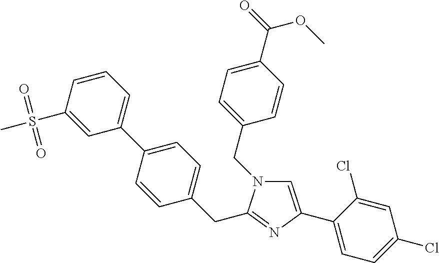 | 4-[4-(2,4-dichloro-phenyl)- 2-(3′-methanesulfonyl- biphenyl-4-ylmethyl)- imidazol-1-ylmethyl]- benzoic acid methyl ester |
| 27 | 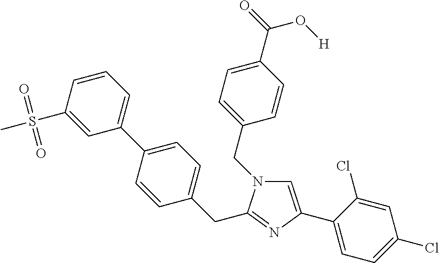 | 4-[4-(2,4-dichloro-phenyl)- 2-(3′-methanesulfonyl- biphenyl-4-ylmethyl)- imidazol-1-ylmethyl]- benzoic acid |
| 28 | 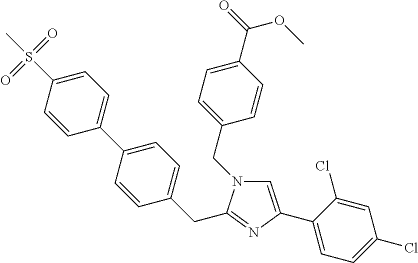 | 4-[4-(2,4-dichloro-phenyl)- 2-(4′-methanesulfonyl- biphenyl-4-ylmethyl)- imidazol-1-ylmethyl]- benzoic acid methyl ester |
| 29 | 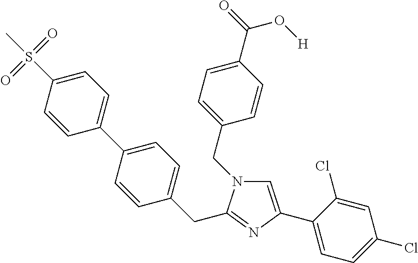 | 4-[4-(2,4-dichloro-phenyl)- 2-(4′-methanesulfonyl- biphenyl-4-ylmethyl)- imidazol-1-ylmethyl]- benzoic acid |
| 30 | 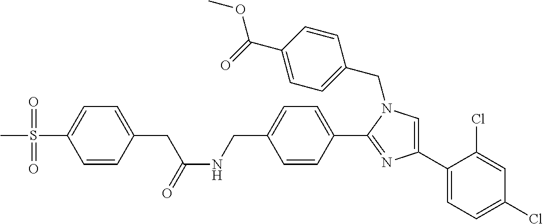 | 4-[4-(2,4-dichloro-phenyl)- 2-(4-{[2-(4- methanesulfonyl-phenyl)- acetylamino]-methyl}- phenyl)-imidazol-1- ylmethyl]-benzoic acid methyl ester |
| 31 | 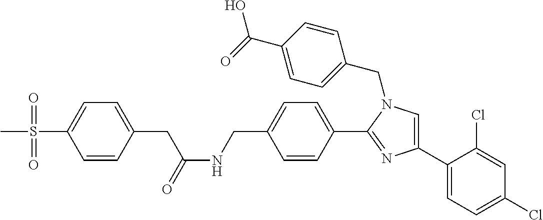 | 4-[4-(2,4-dichloro-phenyl)- 2-(4-{[2-(4- methanesulfonyl-phenyl)- acetylamino]-methyl}- phenyl)-imidazol-1- ylmethyl]-benzoic acid |
| 32 | 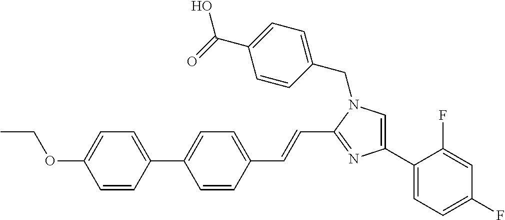 | 4-{4-(2,4-difluoro-phenyl)- 2-[2-(4′-ethoxy-biphenyl- 4-yl)-(E)-vinyl]-imidazol-1- ylmethyl}-benzoic acid |
| 33 | 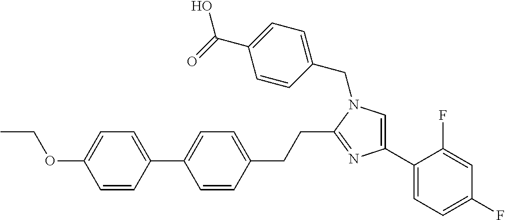 | 4-{4-(2,4-difluoro-phenyl)- 2-[2-(4′-ethoxy-biphenyl- 4-yl)-ethyl]-imidazol-1- ylmethyl}-benzoic acid |
| 34 | 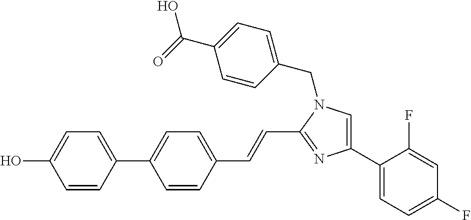 | 4-{4-(2,4-difluoro-phenyl)- 2-[2-(4′-hydroxy-biphenyl- 4-yl)-(E)-vinyl]-imidazol-1- ylmethyl}-benzoic acid |
| 35 | 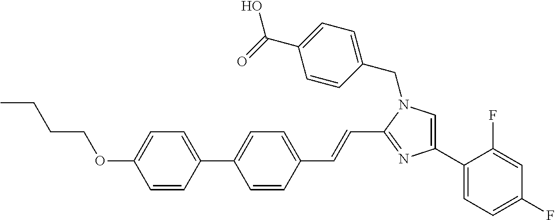 | 4-[2-[2-(4′-butoxy- biphenyl-4-yl)-(E)-vinyl]-4- (2,4-difluoro-phenyl)- imidazol-1-ylmethyl]- benzoic acid |
| 36 | 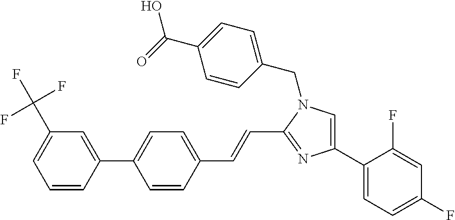 | 4-{4-(2,4-difluoro-phenyl)- 2-[2-(3′-trifluoromethyl- biphenyl-4-yl)-(E)-vinyl]- imidazol-1-ylmethyl}- benzoic acid |
| 37 | 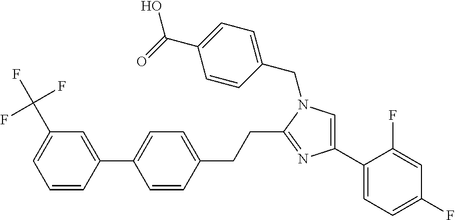 | 4-{4-(2,4-difluoro-phenyl)- 2-[2-(3′-trifluoromethyl- biphenyl-4-yl)-ethyl]- imidazol-1-ylmethyl}- benzoic acid |
| 38 | 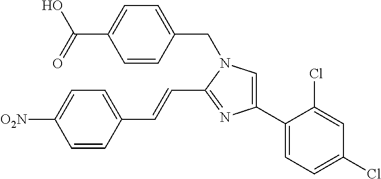 | 4-{4-(2,4-dichloro-phenyl)- 2-[2-(4-nitro-phenyl)-(E)- vinyl]-imidazol-1- ylmethyl}-benzoic acid |
| 39 | 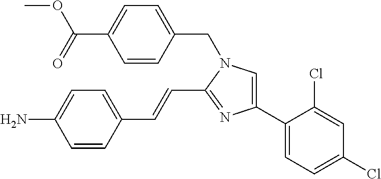 | 4-[2-[2-(4-amino-phenyl)- (E)-vinyl]-4-(2,4-dichloro- phenyl)-imidazol-1- ylmethyl]-benzoic acid methyl ester |
| 40 | 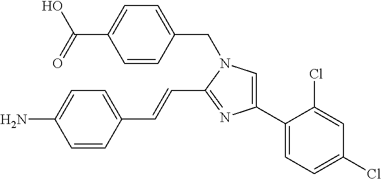 | 4-[2-[2-(4-amino-phenyl)- (E)-vinyl]-4-(2,4-dichloro- phenyl)-imidazol-1- ylmethyl]-benzoic acid |
| 41 | 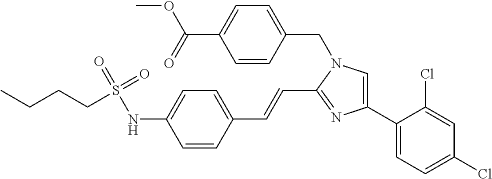 | 4-[2-{2-[4-(butane-1- sulfonylamino)-phenyl]- (E)-vinyl}-4-(2,4-dichloro- phenyl)-imidazol-1- ylmethyl]-benzoic acid methyl ester |
| 42 | 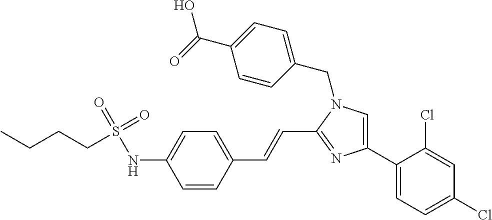 | 4-[2-{2-[4-(butane-1- sulfonylamino)-phenyl]- (E)-vinyl}-4-(2,4-dichloro- phenyl)-imidazol-1- ylmethyl]-benzoic acid |
| 43 | 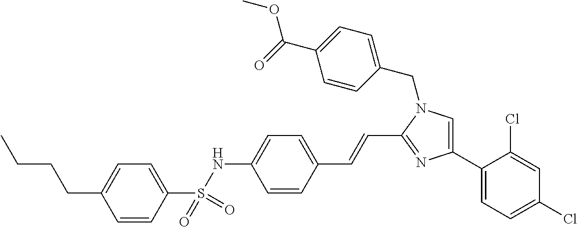 | 4-[2-{2-[4-(4-butyl- benzenesulfonylamino)- phenyl]-(E)-vinyl}-4-(2,4- dichloro-phenyl)-imidazol- 1-ylmethyl]-benzoic acid methyl ester |
| 44 | 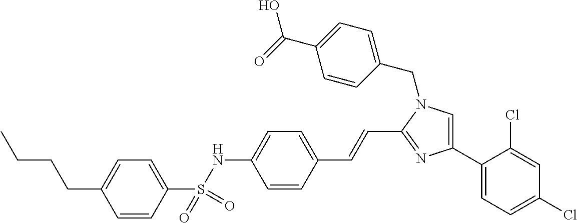 | 4-[2-{2-[4-(4-butyl- benzenesulfonylamino)- phenyl]-(E)-vinyl}-4-(2,4- dichloro-phenyl)-imidazol- 1-ylmethyl]-benzoic acid |
| 45 | 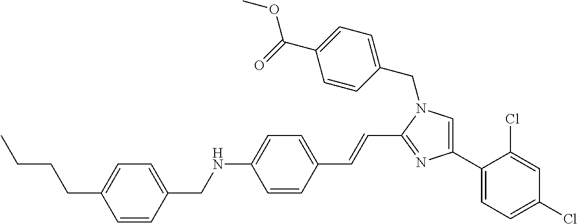 | 4-[2-{2-[4-(4-butyl- benzylamino)-phenyl]-(E)- vinyl}-4-(2,4-dichloro- phenyl)-imidazol-1- ylmethyl]-benzoic acid methyl ester |
| 46 | 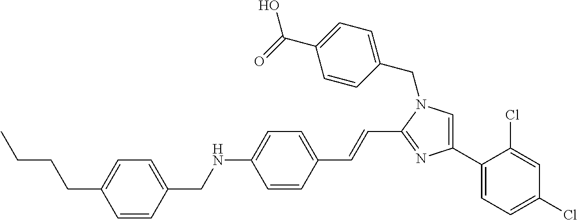 | 4-[2-{2-[4-(4-butyl- benzylamino)-phenyl]-(E)- vinyl}-4-(2,4-dichloro- phenyl)-imidazol-1- ylmethyl]-benzoic acid |
| 47 | 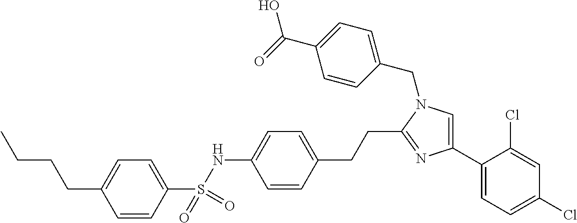 | 4-[2-{2-[4-(4-butyl- benzenesulfonylamino)- phenyl]-ethyl}-4-(2,4- dichloro-phenyl)-imidazol- 1-ylmethyl]-benzoic acid |
| 48 | 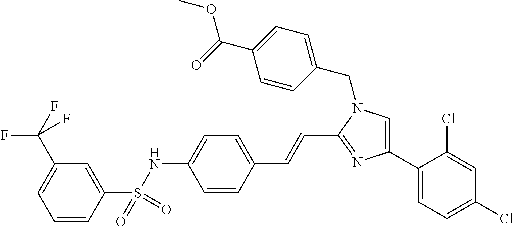 | 4-(4-(2,4-dichloro-phenyl)- 2-{2-[4-(3-trifluoromethyl- benzenesulfonylamino)- phenyl]-(E)-vinyl}- imidazol-1-ylmethyl)- benzoic acid methyl ester |
| 49 | 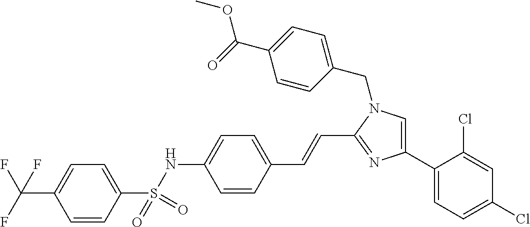 | 4-(4-(2,4-dichloro-phenyl)- 2-{2-[4-(4-trifluoromethyl- benzenesulfonylamino)- phenyl]-(E)-vinyl}- imidazol-1-ylmethyl)- benzoic acid methyl ester |
| 50 | 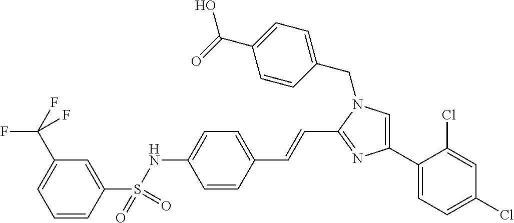 | 4-(4-(2,4-dichloro-phenyl)- 2-{2-[4-(3-trifluoromethyl- benzenesulfonylamino)- phenyl]-(E)-vinyl}- imidazol-1-ylmethyl)- benzoic acid |
| 51 | 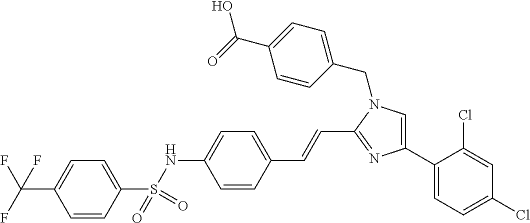 | 4-(4-(2,4-dichloro-phenyl)- 2-{2-[4-(4-trifluoromethyl- benzenesulfonylamino)- phenyl]-(E)-vinyl}- imidazol-1-ylmethyl)- benzoic acid |
| 52 | 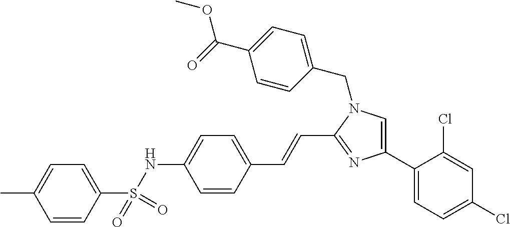 | 4-(4-(2,4-dichloro-phenyl)- 2-{2-[4-(toluene-4- sulfonylamino)-phenyl]- (E)-vinyl}-imidazol-1- ylmethyl)-benzoic acid methyl ester |
| 53 | 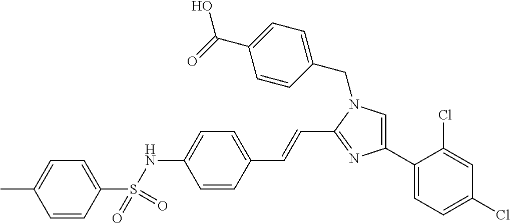 | 4-(4-(2,4-dichloro-phenyl)- 2-{2-[4-(toluene-4- sulfonylamino)-phenyl]- (E)-vinyl}-imidazol-1- ylmethyl)-benzoic acid |
| 54 | 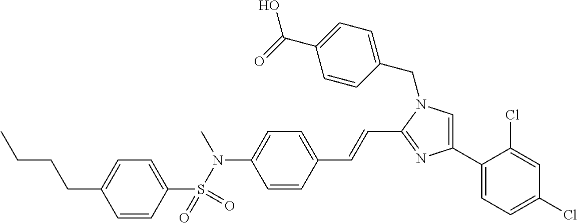 | 4-[2-(2-{4-[(4-butyl- benzenesulfonyl)-methyl- amino]-phenyl}-(E)-vinyl)- 4-(2,4-dichloro-phenyl)- imidazol-1-ylmethyl]- benzoic acid |
| 55 | 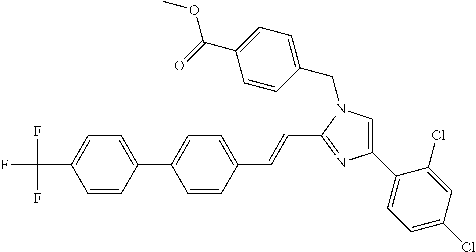 | 4-{4-(2,4-dichloro-phenyl)- 2-[2-(4′-trifluoromethyl- biphenyl-4-yl)-(E)-vinyl]- imidazol-1yl-methyl} benzoic acid methyl ester |
| 56 | 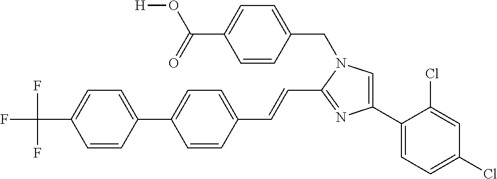 | 4-{4-(2,4-dichloro-phenyl)- 2[2-(4′-trifluoromethyl- biphenyl-4-yl)-(E)-vinyl]- imidazol-1-ylmethyl}- benzoic acid |
| 57 | 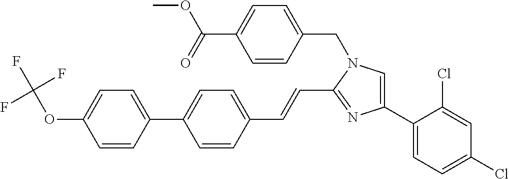 | 4-{4-(2,4-dichloro-phenyl)- 2-[2-(4′-trifluoromethoxy- biphenyl-4-yl)-(E)-vinyl]- imidazol-1yl-methyl} benzoic acid methyl ester |
| 58 | 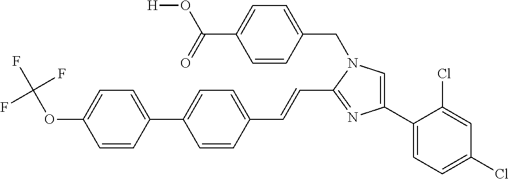 | 4-{4-(2,4-dichloro-phenyl)- 2[2-(4′-trifluoromethoxy- biphenyl-4-yl)-(E)-vinyl]- imidazol-1-ylmethyl}- benzoic acid |
| 59 | 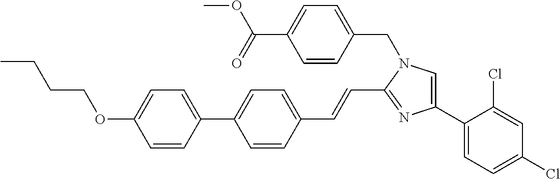 | 4-[2-[2-(4′-butoxy- biphenyl-4-yl)-(E)-vinyl]-4- (2,4-dichloro-phenyl)- imidazol-1-ylmethyl]- benzoic acid methyl ester |
| 60 | 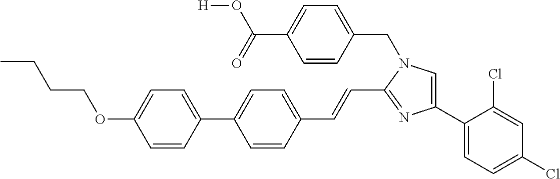 | 4-[2-[2-(4′-butoxy- biphenyl-4-yl)-(E)-vinyl]-4- (2,4-dichloro-phenyl)- imidazol-1-ylmethyl]- benzoic acid |
| 61 | 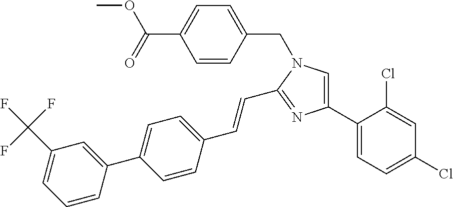 | 4-{4-(2,4-dichloro-phenyl)- 2-[2-(3′-trifluoromethyl- biphenyl-4-yl)-(E)-vinyl]- imidazol-1yl-methyl} benzoic acid methyl ester |
| 62 | 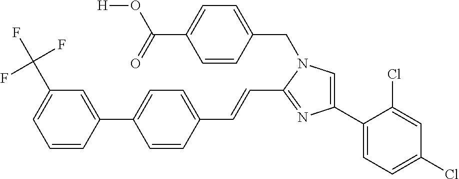 | 4-{4-(2,4-dichloro-phenyl)- 2[2-(3′-trifluoromethyl- biphenyl-4-yl)-(E)-vinyl]- imidazol-1-ylmethyl}- benzoic acid |
| 63 | 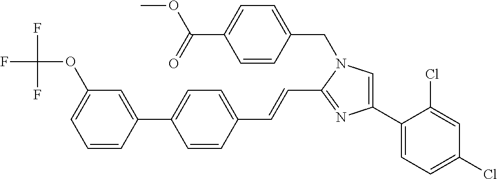 | 4-{4-(2,4-dichloro-phenyl)- 2-[2-(3′-trifluoromethoxy- biphenyl-4-yl)-(E)-vinyl]- imidazol-1yl-methyl} benzoic acid methyl ester |
| 64 | 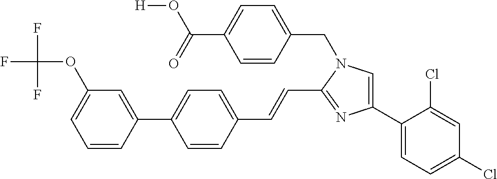 | 4-{4-(2,4-dichloro-phenyl)- 2-[2-(3′-trifluoromethoxy- biphenyl-4-yl)-(E)-vinyl]- imidazol-1-ylmethyl}- benzoic acid |
| 65 | 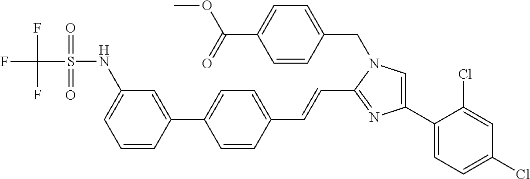 | 4-{4-(2,4-dichloro-phenyl)- 2[2-(3- trifluoromethanesulfonyl amino-biphenyl-4-yl)-(E)- vinyl]-imidazol-1- ylmethyl}-benzoic acid methyl ester |
| 66 | 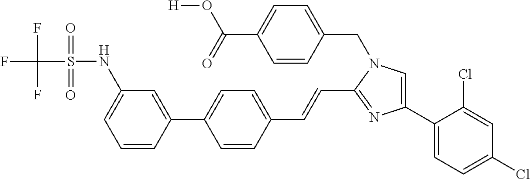 | 4-{4-(2,4-dichloro-phenyl)- 2-[2-(3′- trifluoromethanesulfonyl amino-biphenyl-4-yl)-(E)- vinyl]-imidazol-1- ylmethyl}-benzoic acid |
| 67 | 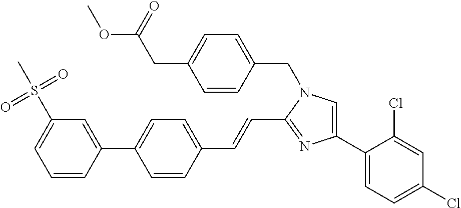 | (4-{4-(2,4-dichloro- phenyl)-2-[2-(3′- methanesulfonyl- biphenyl-4-yl)-(E)-vinyl]- imidazol-1-ylmethyl}- phenyl)-acetic acid methyl ester |
| 68 | 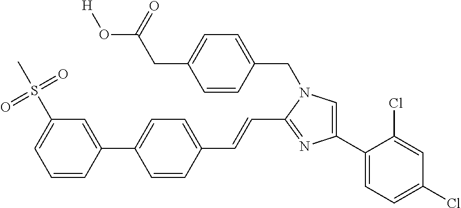 | (4-{4-(2,4-dichloro- phenyl)-2-[2-(3′- methanesulfonyl- biphenyl-4-yl)-(E)-vinyl]- imidazol-1-ylmethyl}- phenyl)-acetic acid |
| 69 | 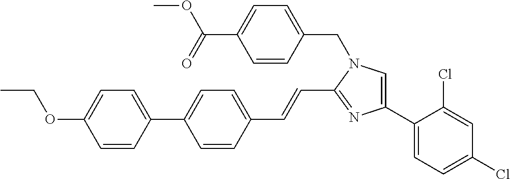 | 4-{4-(2,4-dichloro-phenyl)- 2-[2-(4′-ethoxy-biphenyl- 4-yl)-(E)-vinyl]-imidazol-1- ylmethyl}-benzoic acid methyl ester |
| 70 | 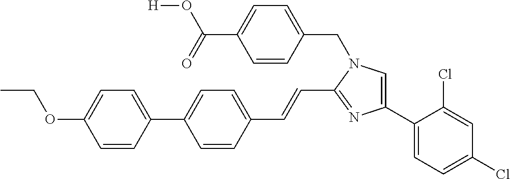 | 4-{4-(2,4-dichloro-phenyl)- 2-[2-(4′-ethoxy-biphenyl- 4-yl)-(E)-vinyl]-imidazol-1- ylmethyl}-benzoic acid |
| 71 | 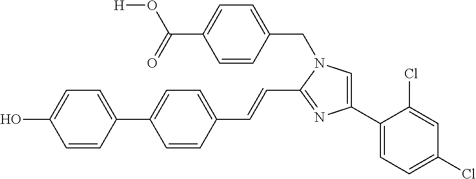 | 4-{4-(2,4-dichloro-phenyl)- 2-[2-(4′-hydroxy-biphenyl- 4-yl-(E)-vinyl]-imidazol-1- ylmethyl}-benzoic acid |
| 72 | 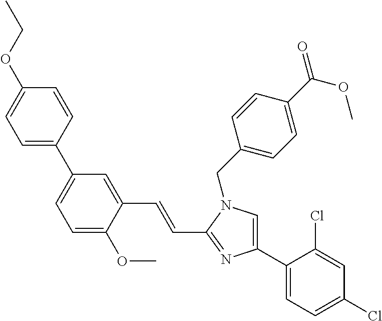 | 4-{4-(2,4-dichloro-phenyl)- 2-[2-(4′-ethoxy-4- methoxy-biphenyl-3-yl)- (E)-vinyl]-imidazol-1- ylmethyl}-benzoic acid methyl ester |
| 73 | 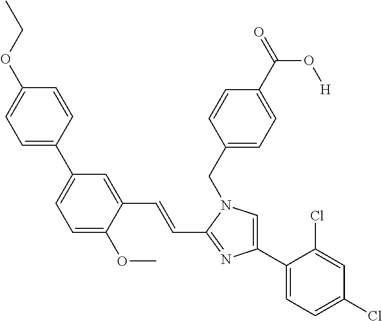 | 4-{4-(2,4-dichloro-phenyl)- 2-[2-(4′-ethoxy-4- methoxy-biphenyl-3-yl)- (E)-vinyl]-imidazol-1- ylmethyl}-benzoic acid |
| 74 | 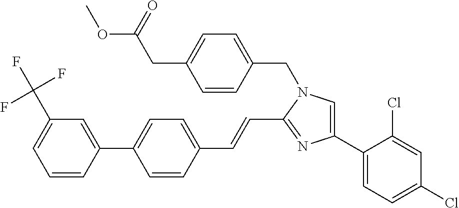 | (4-{4-(2,4-dichloro- phenyl)-2-[2-(3′- trifluoromethyl-biphenyl-4- yl)-(E)-vinyl]-imidazol-1- ylmethyl}-phenyl)-acetic acid methyl ester |
| 75 | 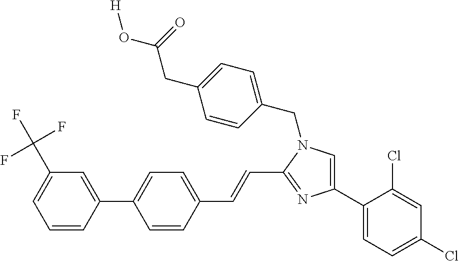 | (4-{4-(2,4-dichloro- phenyl)-2-[2-(3′- trifluoromethyl-biphenyl-4- yl-(E)-vinyl]-imidazol-1- ylmethyl}-phenyl)-acetic acid |
| 76 | 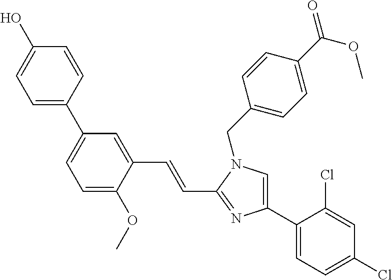 | 4-{4-(2,4-dichloro-phenyl)- 2-[2-(4′-hydroxy-4- methoxy-biphenyl-3-yl)- (E)-vinyl]-imidazol-1- ylmethyl}-benzoic acid methyl ester |
| 77 | 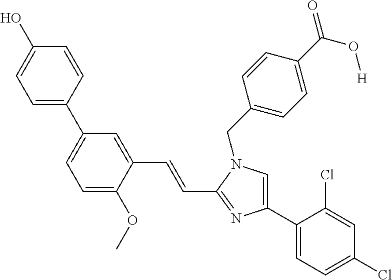 | 4-{4-(2,4-dichloro-phenyl)- 2-[2-(4′-hydroxy-4- methoxy-biphenyl-3-yl)- (E)-vinyl]-imidazol-1- ylmethyl}-benzoic acid |
| 78 | 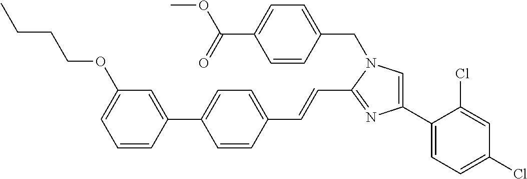 | 4-[2-[2-(3′-butoxy- biphenyl-4-yl)-(E)-vinyl]-4- (2,4-dichloro-phenyl)- imidazol-1-ylmethyl]- benzoic acid methyl ester |
| 79 | 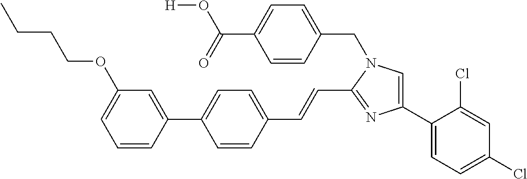 | 4-[2-[2-(3′-butoxy- biphenyl-4-yl)-(E)-vinyl]-4- (2,4-dichloro-phenyl)- imidazol-1-ylmethyl]- benzoic acid |
| 80 | 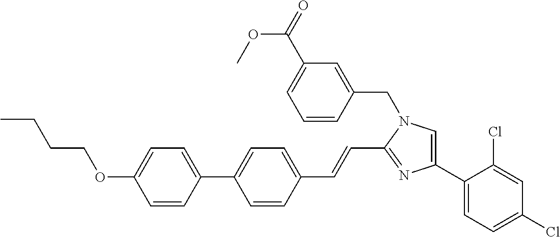 | 3-[2-[2-(4′-butoxy- biphenyl-4-yl)-(E)-vinyl]-4- (2,4-dichloro-phenyl)- imidazol-1-ylmethyl]- benzoic acid methyl ester |
| 81 | 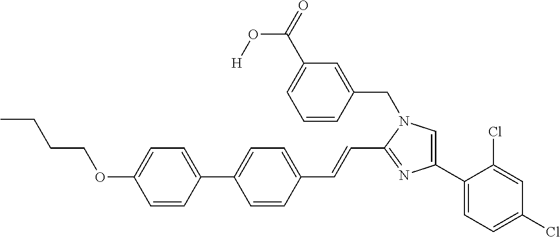 | 3-[2-[2-(4′-butoxy- biphenyl-4-yl)-(E)-vinyl]-4- (2,4-dichloro-phenyl)- imidazol-1-ylmethyl]- benzoic acid |
| 82 | 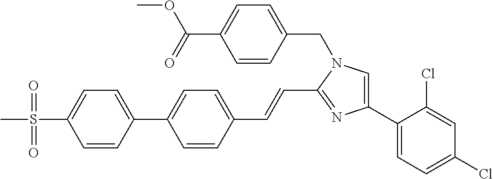 | 4-{4-(2,4-dichloro-phenyl)- 2-[2-(4′-methanesulfonyl- biphenyl-4-yl)-(E)-vinyl]- imidazol-1-ylmethyl}- benzoic acid methyl ester |
| 83 | 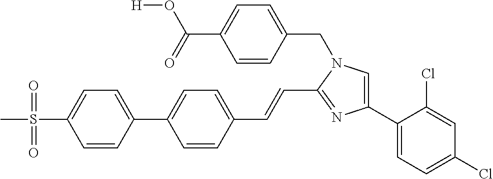 | 4-{4-(2,4-dichloro-phenyl)- 2-[2-(4′-methanesulfonyl- biphenyl-4-yl)-(E)-vinyl]- imidazol-1-ylmethyl}- benzoic acid |
| 84 | 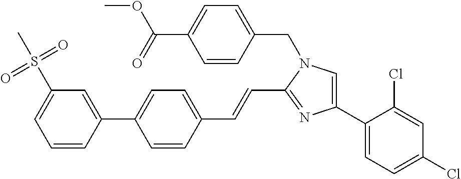 | 4-{4-(2,4-dichloro-phenyl)- 2-[2-(3′-methanesulfonyl- biphenyl-4-yl)-(E)-vinyl]- imidazol-1-ylmethyl}- benzoic acid methyl ester |
| 85 | 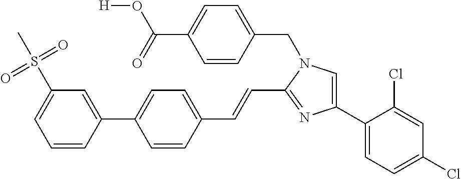 | 4-{4-(2,4-dichloro-phenyl)- 2-[2-(3′-methanesulfonyl- biphenyl-4-yl)-(E)-vinyl]- imidazol-1-ylmethyl}- benzoic acid |
| 86 | 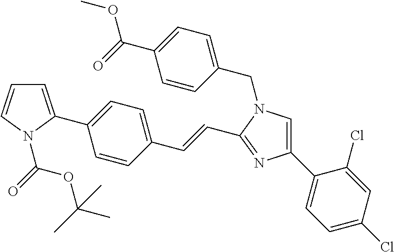 | 2-(4-{2-[4-(2,4-dichloro- phenyl)-1-(4- methoxycarbonyl-benzyl)- 1H-imidazol-2-yl]-(E)- vinyl}-phenyl)-pyrrole-1- carboxylic acid tert-butyl ester |
| 87 | 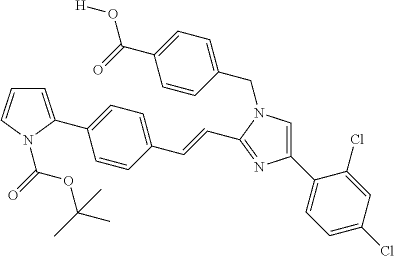 | 2-(4-{2-[1-(4-carboxy- benzyl)-4-(2,4-dichloro- phenyl)-1H-imidazol-2-yl]- (E)-vinyl}-phenyl)-pyrrole- 1-carboxylic acid tert-butyl ester |
| 88 | 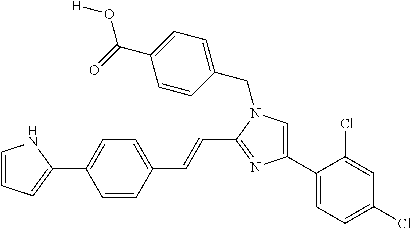 | 4-(4-(2,4-dichloro-phenyl)- 2-{2-[4-(1H-pyrrol-2-yl)- phenyl]-(E)-vinyl}- imidazol-1-ylmethyl)- benzoic acid |
| 89 | 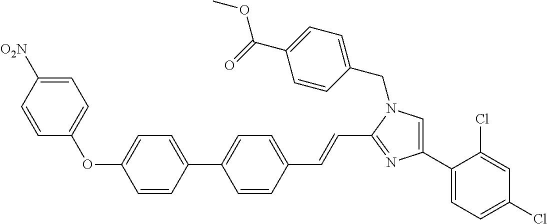 | 4-[2-{2-[4′-(4-nitro- phenoxy)-biphenyl-4-yl]- (E)-vinyl}-4-(2,4-dichloro- phenyl)-imidazol-1- ylmethyl]-benzoic acid methyl ester |
| 90 | 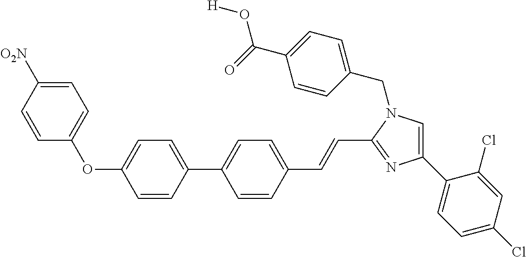 | 4-[2-{2-[4′-(4-nitro- phenoxy)-biphenyl-4-yl]- (E)-vinyl}-4-(2,4-dichloro- phenyl)-imidazol-1- ylmethyl]-benzoic acid |
| 91 | 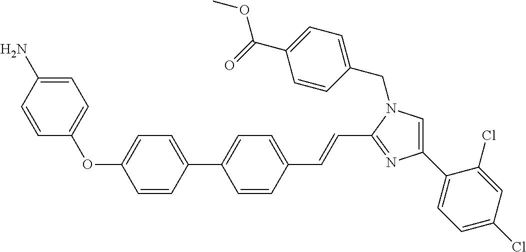 | 4-[2-{2-[4′-(4-amino- phenoxy)-biphenyl-4-yl]- (E)-vinyl}-4-(2,4-dichloro- phenyl)-imidazol-1- ylmethyl]-benzoic acid methyl ester |
| 92 | 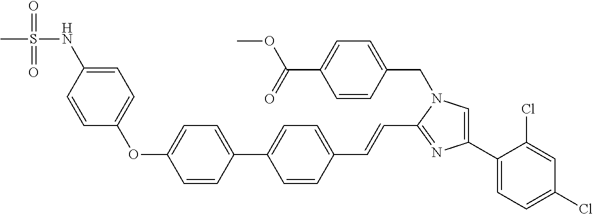 | 4-(4-(2,4-dichloro-phenyl)- 2-{2-[4′-(4- methanesulfonylamino- phenoxy)-biphenyl-4-yl]- (E)-vinyl}-imidazol-1- ylmethyl)-benzoic acid methyl ester |
| 93 | 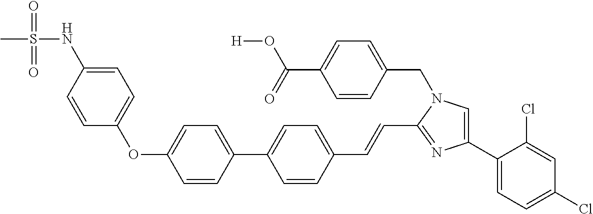 | 4-(4-(2,4-dichloro-phenyl)- 2-{2-[4′-(4- methanesulfonylamino- phenoxy)-biphenyl-4-yl]- (E)-vinyl}-imidazol-1- ylmethyl)-benzoic acid |
| 94 | 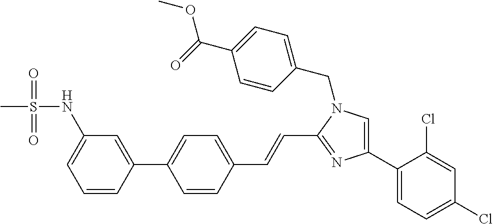 | 4-{4-(2,4-dichloro-phenyl)- 2-[2-(3′- methanesulfonylamino- biphenyl-4-yl)-(E)-vinyl]- imidazol-1-ylmethyl}- benzoic acid methyl ester |
| 95 | 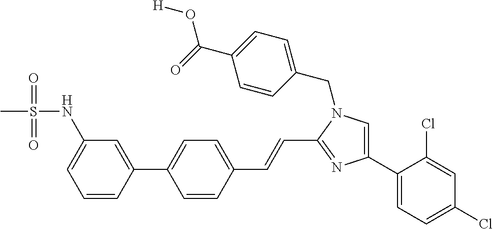 | 4-{4-(2,4-dichloro-phenyl)- 2-[2-(3′- methanesulfonylamino- biphenyl-4-yl)-(E)-vinyl]- imidazol-1-ylmethyl}- benzoic acid |
| 96 | 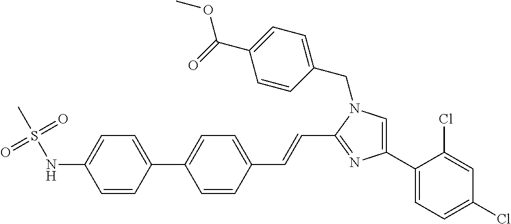 | 4-{4-(2,4-dichloro-phenyl)- 2-[2-(4′- methanesulfonylamino- biphenyl-4-yl)-(E)-vinyl]- imidazol-1-ylmethyl}- benzoic acid methyl ester |
| 97 | 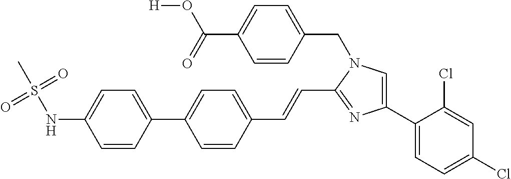 | 4-{4-(2,4-Dichloro- phenyl)-2-[2-(4′- methanesulfonylamino- biphenyl-4-yl)-(E)-vinyl]- imidazol-1-ylmethyl}- benzoic acid |
| 98 | 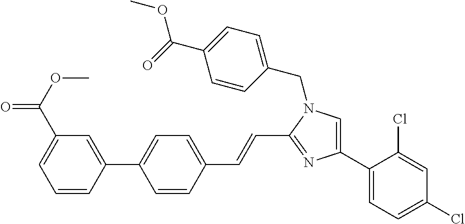 | 4′-{2-[4-(2,4-dichloro- phenyl)-1-(4- methoxycarbonyl-benzyl)- 1H-imidazol-2-yl]-(E)- vinyl}-biphenyl-3- carboxylic acid methyl ester |
| 99 | 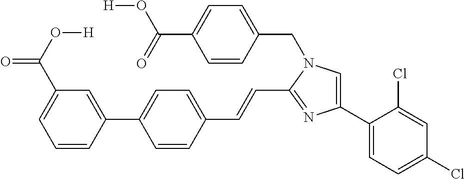 | 4′-{2-[1-(4-carboxy- benzyl)-4-(2,4-dichloro- phenyl)-1H-imidazol-2-yl]- (E)-vinyl}-biphenyl-3- carboxylic acid |
| 100 | 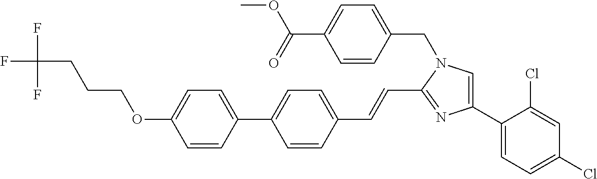 | 4-(4-(2,4-dichloro-phenyl)- 2-{2-[4′-(4,4,4-trifluoro- butoxy)-biphenyl-4-yl]-(E)- vinyl}-imidazol-1- ylmethyl)-benzoic acid methyl ester |
| 101 | 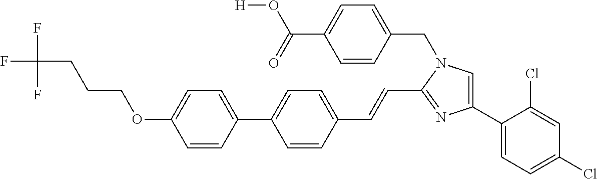 | 4-(4-(2,4-dichloro-phenyl)- 2-{2-[4′-(4,4,4-trifluoro- butoxy)-biphenyl-4-yl]-(E)- vinyl}-imidazol-1- ylmethyl)-benzoic acid |
| 102 | 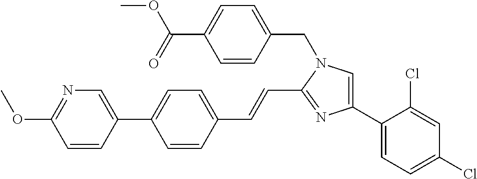 | 4-(4-(2,4-dichloro-phenyl)- 2-{2-[4-(6-methoxy- pyridin-3-yl)-phenyl]-(E)- vinyl}-imidazol-1- ylmethyl)-benzoic acid methyl ester |
| 103 | 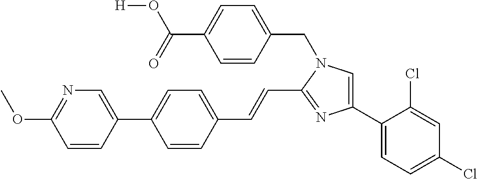 | 4-(4-(2,4-dichloro-phenyl)- 2-{2-[4-(6-methoxy- pyridin-3-yl)-phenyl]-(E)- vinyl}-imidazol-1- ylmethyl)-benzoic acid |
| 104 | 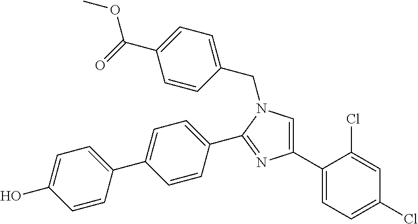 | 4-[4-(2,4-dichloro-phenyl)- 2-(4′-hydroxy-biphenyl-4- yl)-imidazol-1-ylmethyl]- benzoic acid methyl ester |
| 105 | 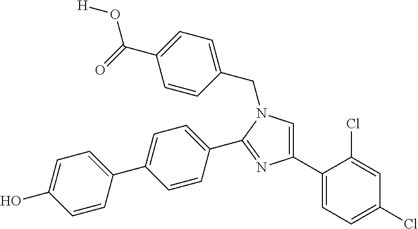 | 4-[4-(2,4-dichloro-phenyl)- 2-(4′-hydroxy-biphenyl-4- yl)-imidazol-1-ylmethyl]- benzoic acid |
| 106 | 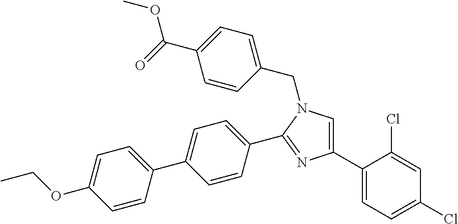 | 4-[4-(2,4-dichloro-phenyl)- 2-(4′-ethoxy-biphenyl-4- yl)-imidazol-1-ylmethyl]- benzoic acid methyl ester |
| 107 | 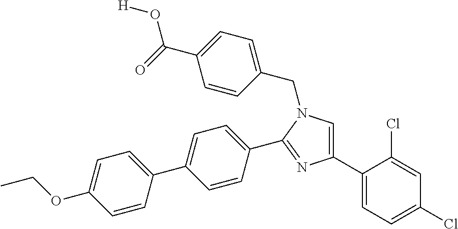 | 4-[4-(2,4-dichloro-phenyl)- 2-(4′-ethoxy-biphenyl-4- yl)-imidazol-1-ylmethyl]- benzoic acid |
| 108 | 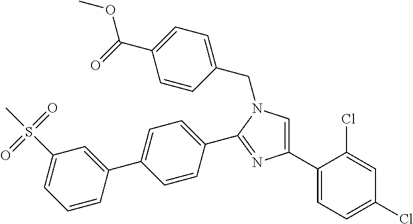 | 4-[4-(2,4-dichloro-phenyl)- 2-(3′-methanesulfonyl- biphenyl-4-yl)-imidazol-1- ylmethyl]-benzoic acid methyl ester |
| 109 | 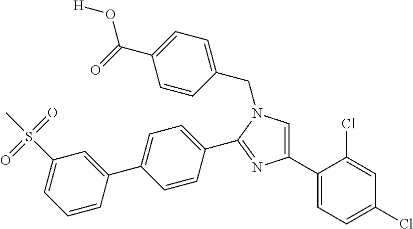 | 4-[4-(2,4-dichloro-phenyl)- 2-(3′-methanesulfonyl- biphenyl-4-yl)-imidazol-1- ylmethyl]-benzoic acid |
| 110 | 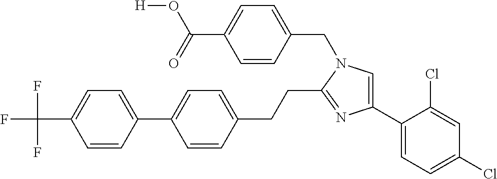 | 4-{4-(2,4-dichloro-phenyl)- 2-[2-(4′-trifluoromethyl- biphenyl-4-yl)-ethyl]- imidazol-1-ylmethyl}- benzoic acid |
| 111 | 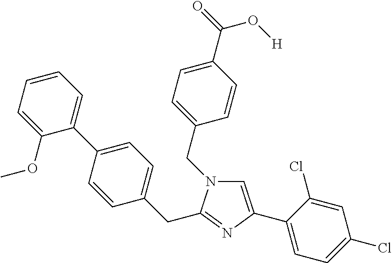 | 4-[4-(2,4-dichloro-phenyl)- 2-(2′-methoxy-biphenyl-4- ylmethyl)-imidazol-1- ylmethyl]-benzoic acid |
| 112 | 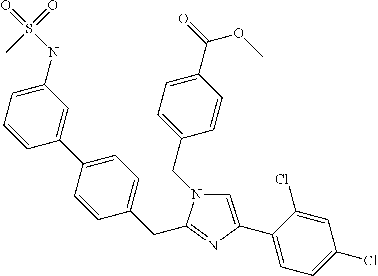 | 4-[4-(2,4-dichloro-phenyl)- 2-(3′- methanesulfonylamino- biphenyl-4-ylmethyl)- imidazol-1-ylmethyl]- benzoic acid methyl ester |
| 113 | 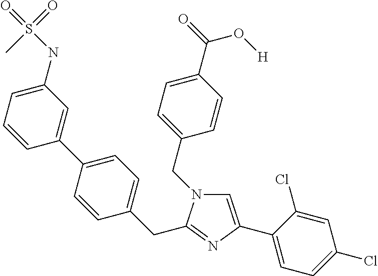 | 4-[4-(2,4-dichloro-phenyl)- 2-(3′- methanesulfonylamino- biphenyl-4-ylmethyl)- imidazol-1-ylmethyl]- benzoic acid |
| 114 | 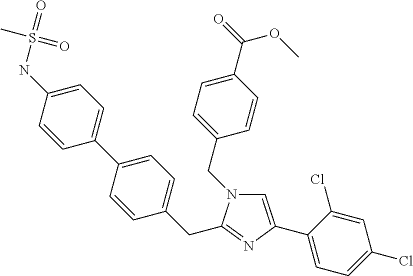 | 4-[4-(2,4-dichloro-phenyl)- 2-(4′- methanesulfonylamino- biphenyl-4-ylmethyl)- imidazol-1-ylmethyl]- benzoic acid methyl ester |
| 115 | 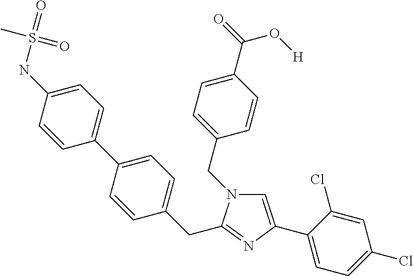 | 4-[4-(2,4-dichloro-phenyl)- 2-(4′- methanesulfonylamino- biphenyl-4-ylmethyl)- imidazol-1-ylmethyl]- benzoic acid |
| 116 | 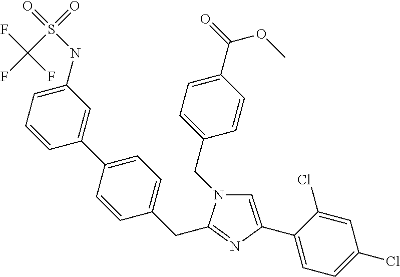 | 4-[4-(2,4-dichloro-phenyl)- 2-(3′- trifluoromethanesulfonyl- amino-biphenyl-4- ylmethyl)-imidazol-1- ylmethyl]-benzoic acid methyl ester |
| 117 | 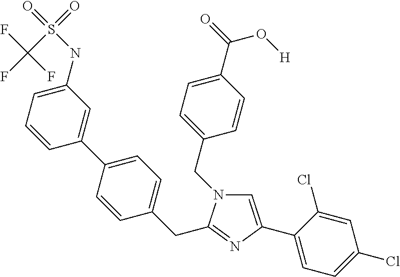 | 4-[4-(2,4-dichloro-phenyl)- 2-(3′- trifluoromethanesulfonyl- amino-biphenyl-4- ylmethyl)-imidazol-1- ylmethyl]-benzoic acid |
| 118 | 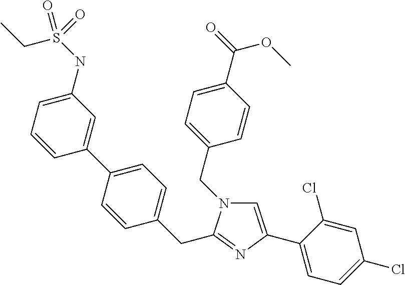 | 4-[4-(2,4-dichloro-phenyl)- 2-(3′- ethanesulfonylamino- biphenyl-4-ylmethyl)- imidazol-1-ylmethyl]- benzoic acid methyl ester |
| 119 | 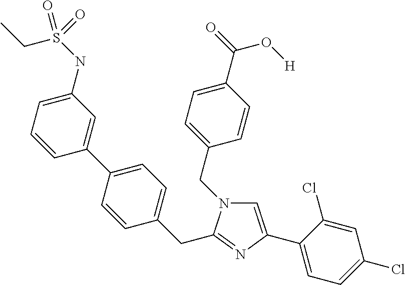 | 4-[4-(2,4-dichloro-phenyl)- 2-(3′- ethanesulfonylamino- biphenyl-4-ylmethyl)- imidazol-1-ylmethyl]- benzoic acid |
| 120 | 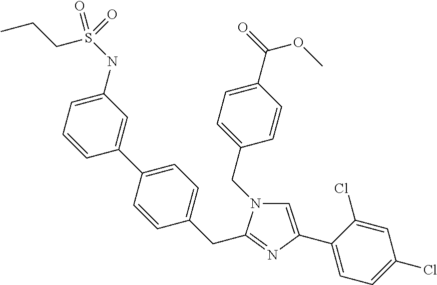 | 4-[4-(2,4-dichloro-phenyl)- 2-(3′- propanesulfonylamino- biphenyl-4-ylmethyl)- imidazol-1-ylmethyl]- benzoic acid methyl ester |
| 121 | 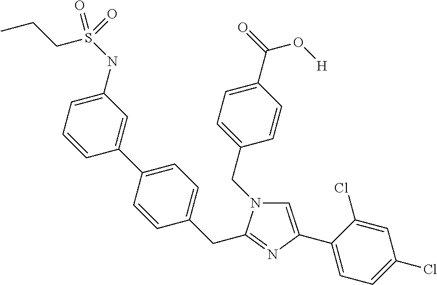 | 4-[4-(2,4-dichloro-phenyl)- 2-(3′- propanesulfonylamino- biphenyl-4-ylmethyl)- imidazol-1-ylmethyl]- benzoic acid |
| 122 | 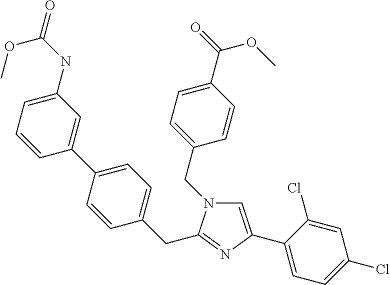 | 4-[4-(2,4-dichloro-phenyl)- 2-(3′- methoxycarbonylamino- biphenyl-4-ylmethyl)- imidazol-1-ylmethyl]- benzoic acid methyl ester |
| 123 | 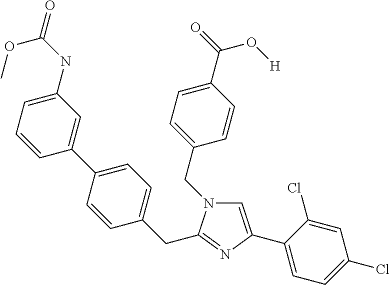 | 4-[4-(2,4-dichloro-phenyl)- 2-(3′- methoxycarbonylamino- biphenyl-4-ylmethyl)- imidazol-1-ylmethyl]- benzoic acid |
| 124 | 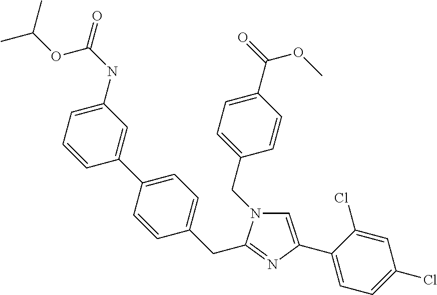 | 4-[4-(2,4-dichloro-phenyl)- 2-(3′- isopropoxycarbonylamino- biphenyl-4-ylmethyl)- imidazol-1-ylmethyl]- benzoic acid methyl ester |
| 125 | 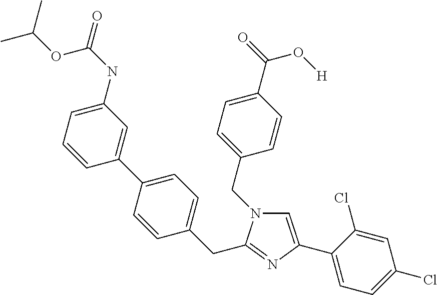 | 4-[4-(2,4-dichloro-phenyl)- 2-(3′- isopropoxycarbonylamino- biphenyl-4-ylmethyl)- imidazol-1-ylmethyl]- benzoic acid |
| 126 | 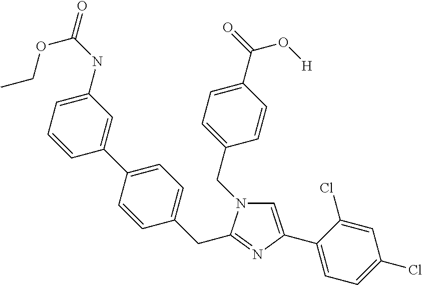 | 4-[4-(2,4-dichloro-phenyl)- 2-(3′- ethoxycarbonylamino- biphenyl-4-ylmethyl)- imidazol-1-ylmethyl]- benzoic acid |
| 127 | 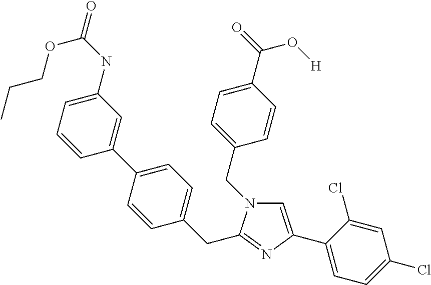 | 4-[4-(2,4-dichloro-phenyl)- 2-(3′- propoxycarbonylamino- biphenyl-4-ylmethyl)- imidazol-1-ylmethyl]- benzoic acid |
| 128 | 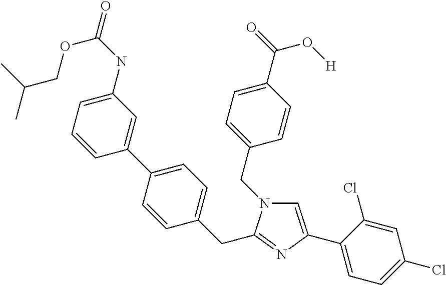 | 4-[4-(2,4-dichloro-phenyl)- 2-(3′- isobutoxycarbonylamino- biphenyl-4-ylmethyl)- imidazol-1-ylmethyl]- benzoic acid |
| 129 | 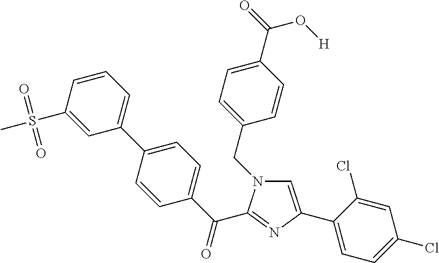 | 4-[4-(2,4-dichloro-phenyl)- 2-(3′-methanesulfonyl- biphenyl-4-carbonyl)- imidazol-1-ylmethyl]- benzoic acid |
| 130 | 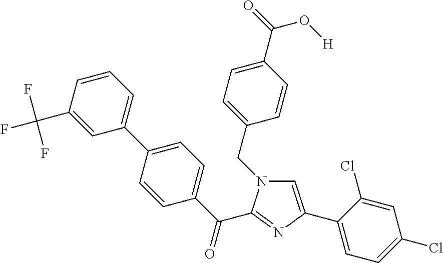 | 4-[4-(2,4-dichloro-phenyl)- 2-(3′-trifluoromethyl- biphenyl-4-carbonyl)- imidazol-1-ylmethyl]- benzoic acid |
| 131 | 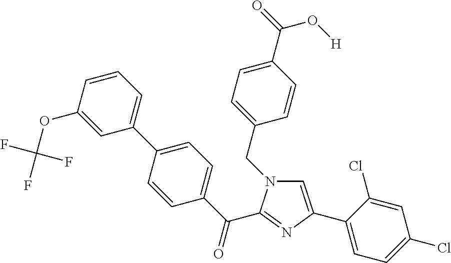 | 4-[4-(2,4-dichloro-phenyl)- 2-(3′-trifluoromethoxy- biphenyl-4-carbonyl)- imidazol-1-ylmethyl]- benzoic acid |
| 132 | 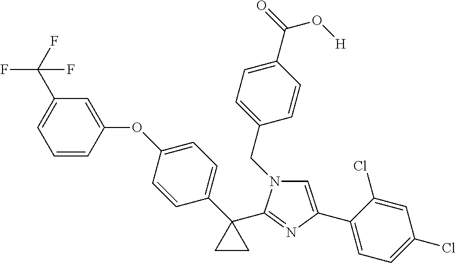 | 4-(4-(2,4-dichloro-phenyl)- 2-{1-[4-(3-trifluoromethyl- phenoxy)-phenyl]- cyclopropyl}-imidazol-1- ylmethyl)-benzoic acid |
| 133 | 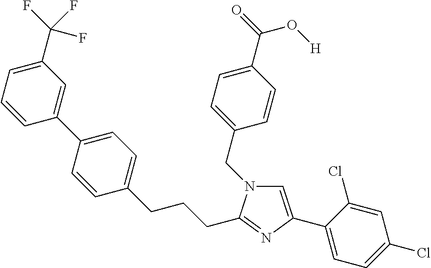 | 4-{4-(2,4-dichloro-phenyl)- 2-[3-(3′-trifluoromethyl- biphenyl-4-yl)-propyl]- imidazol-1-ylmethyl}- benzoic acid |
| 134 | 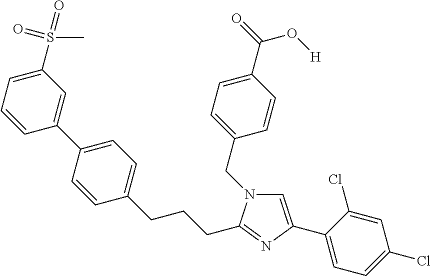 | 4-{4-(2,4-dichloro-phenyl)- 2-[3-(3′-methanesulfonyl- biphenyl-4-yl)-propyl]- imidazol-1-ylmethyl}- benzoic acid |
| 135 | 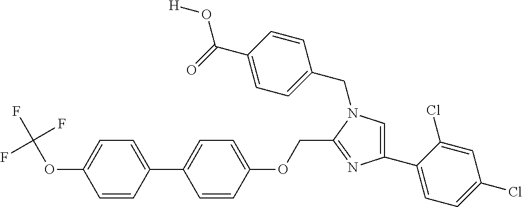 | 4-[4-(2,4-dichloro-phenyl)- 2-(4′-trifluoromethoxy- biphenyl-4-yloxymethyl)- imidazol-1-ylmethyl]- benzoic acid |
| 136 | 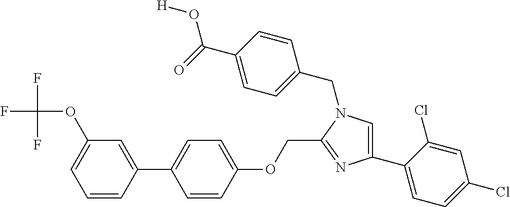 | 4-[4-(2,4-dichloro-phenyl)- 2-(3′-trifluoromethoxy- biphenyl-4-yloxymethyl)- imidazol-1-ylmethyl]- benzoic acid |
| 137 | 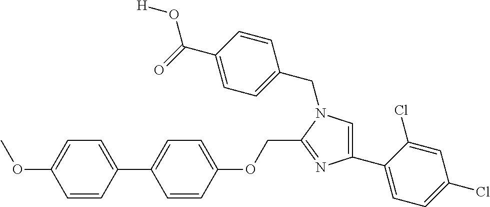 | 4-[4-(2,4-dichloro-phenyl)- 2-(4′-methoxy-biphenyl-4- yloxymethyl)-imidazol-1- ylmethyl]-benzoic acid |
| 138 | 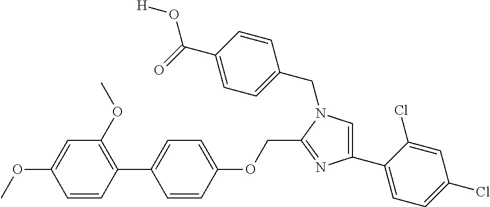 | 4-[4-(2,4-dichloro-phenyl)- 2-(2′,4′-dimethoxy- biphenyl-4-yloxymethyl)- imidazol-1-ylmethyl]- benzoic acid |
| 139 | 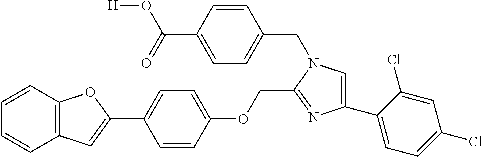 | 4-[2-(4-benzofuran-2-yl- phenoxymethyl)-4-(2,4- dichloro-phenyl)-imidazol- 1-ylmethyl]-benzoic acid |
| 140 | 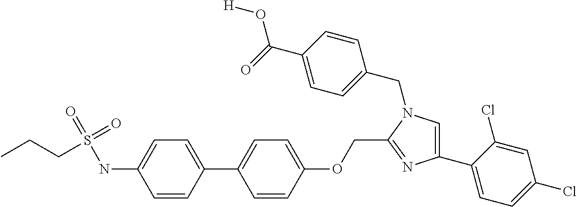 | 4-{4-(2,4-dichloro-phenyl)- 2-[4′-(propane-1- sulfonylamino)-biphenyl- 4-yloxymethyl]-imidazol-1- ylmethyl}-benzoic acid |
| 141 | 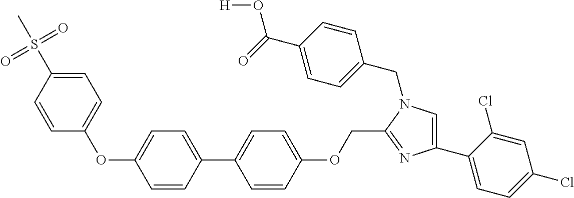 | 4-{4-(2,4-dichloro-phenyl)- 2-[4′-(4-methanesulfonyl- phenoxy)-biphenyl-4- yloxymethyl]-imidazol-1- ylmethyl}-benzoic acid |
| 142 | 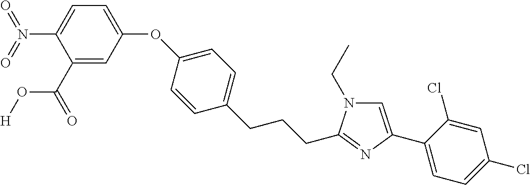 | 5-(4-{3-[4-(2,4-dichloro- phenyl)-1-ethyl-1H- imidazol-2-yl]-propyl}- phenoxy)-2-nitro-benzoic acid |
| 143 | 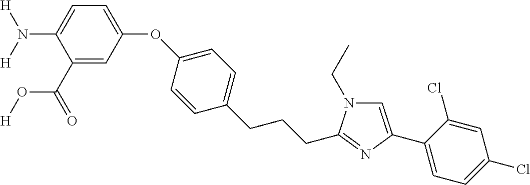 | 2-amino-5-(4-{3-[4-(2,4- dichloro-phenyl)-1-ethyl- 1H-imidazol-2-yl]-propyl}- phenoxy)-benzoic acid |
| 144 | 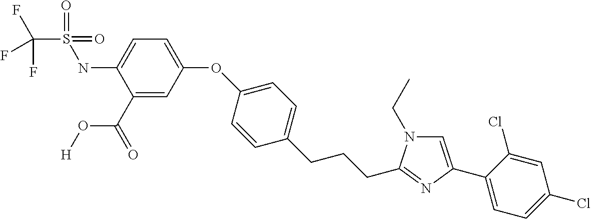 | 5-(4-{3-[4-(2,4-dichloro- phenyl)-1-ethyl-1H- imidazol-2-yl]-propyl}- phenoxy)-2- trifluoromethanesulfonyl- amino-benzoic acid |
| 145 | 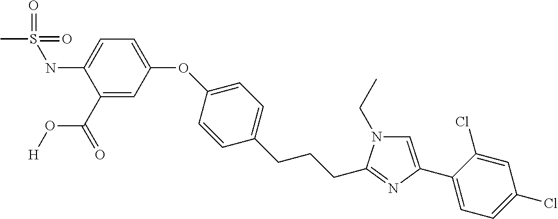 | 5-(4-{3-[4-(2,4-dichloro- phenyl)-1-ethyl-1H- imidazol-2-yl]-propyl}- phenoxy)-2- methanesulfonylamino- benzoic acid |
| 146 | 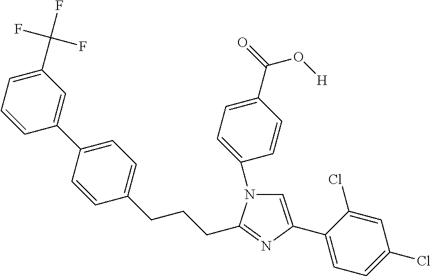 | 4-{4-(2,4-dichloro-phenyl)- 2-[3-(3′-trifluoromethyl- biphenyl-4-yl)-propyl]- imidazol-1-yl}-benzoic acid |
| 147 | 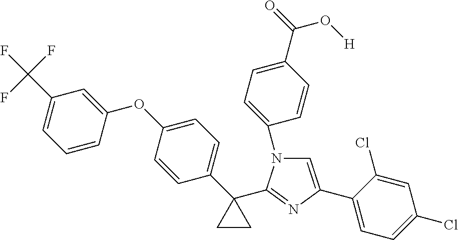 | 4-(4-(2,4-dichloro-phenyl)- 2-{1-[4-(3-trifluoromethyl- phenoxy)-phenyl]- cyclopropyl}-imidazol-1- yl)-benzoic acid |
| 148 | 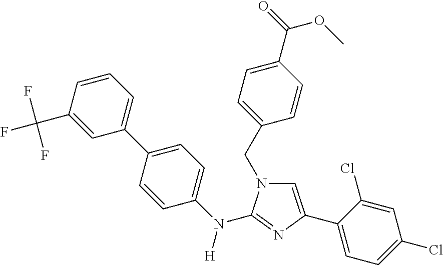 | 4-[4-(2,4-dichloro-phenyl)- 2-(3′-trifluoromethyl- biphenyl-4-ylamino)- imidazol-1-ylmethyl]- benzoic acid methyl ester |
| 149 | 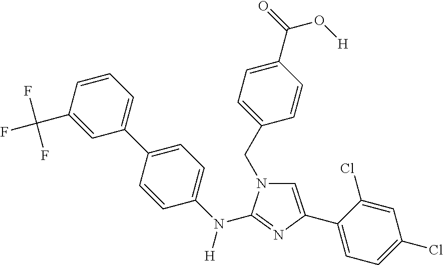 | 4-[4-(2,4-dichloro-phenyl)- 2-(3′-trifluoromethyl- biphenyl-4-ylamino)- imidazol-1-ylmethyl]- benzoic acid |
| 150 | 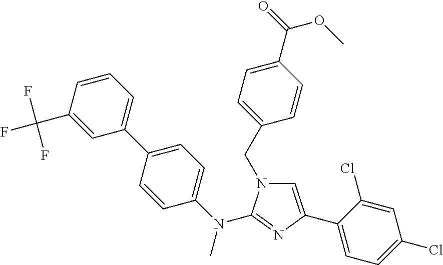 | 4-{4-(2,4-dichloro-phenyl)- 2-[methyl-(3′- trifluoromethyl-biphenyl-4- yl)-amino]-imidazol-1- ylmethyl}-benzoic acid methel ester |
| 151 | 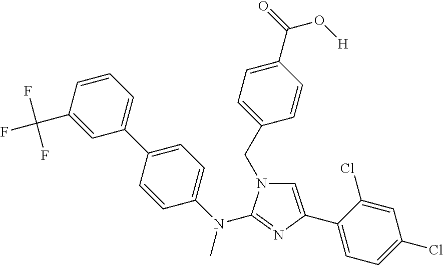 | 4-{4-(2,4-dichloro-phenyl)- 2-[methyl-(3′- trifluoromethyl-biphenyl-4- yl)-amino]-imidazol-1- ylmethyl}-benzoic acid |
| 152 | 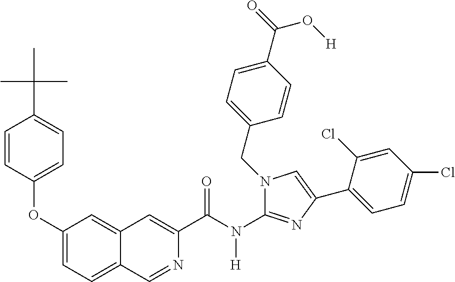 | 4-[2-{[6-(4-tert-butyl- phenoxy)-isoquinoline-3- carbonyl]-amino}-4-(2,4- dichloro-phenyl)-imidazol- 1-ylmethyl]-benzoic acid |
| 153 | 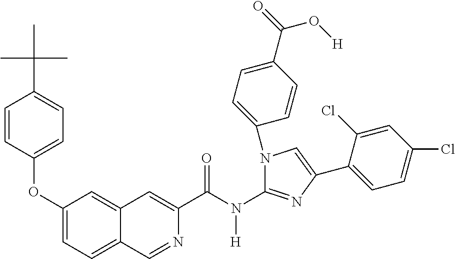 | 4-[2-{[6-(4-tert-butyl- phenoxy)-isoquinoline-3- carbonyl]-amino}-4-(2,4- dichloro-phenyl)-imidazol- 1-yl]-benzoic acid |
| 154 | 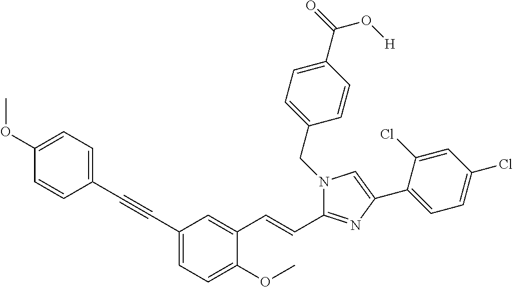 | 4-(4-(2,4-dichloro-phenyl)- 2-{2-[2-methoxy-5-(4- methoxy-phenylethynyl)- phenyl]-(E)-vinyl}- imidazol-1-ylmethyl)- benzoic acid |
| 155 | 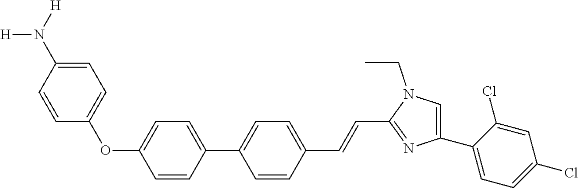 | 4-(4′-{2-[4-(2,4-dichloro- phenyl)-1-ethyl-1H- imidazol-2-yl]-(E)-vinyl}- biphenyl-4-yloxy)- phenylamine |
| 156 | 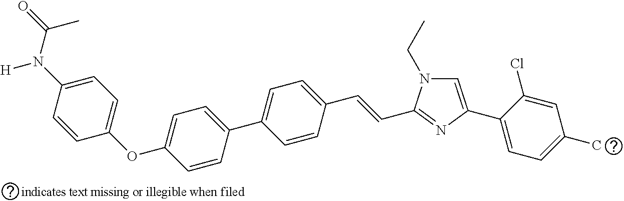 | N-[4-(4′-{2-[4-(2,4- dichloro-phenyl)-1-ethyl- 1H-imidazol-2-yl]-(E)- vinyl}-biphenyl-4-yloxy)- phenyl]-acetamide |
| 157 | 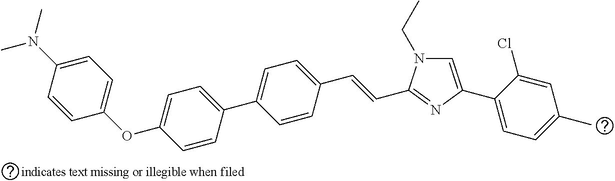 | [4-(4′-{2-[4-(2,4-dichloro- phenyl)-1-ethyl-1H- imidazol-2-yl]-(E)-vinyl}- biphenyl-4-yloxy)-phenyl]- dimethyl-amine |
| 158 | 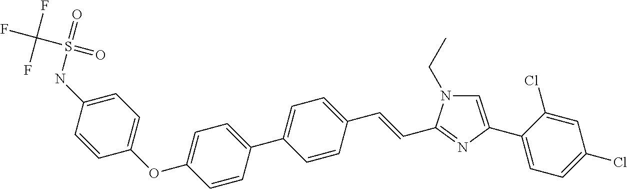 | N-[4-(4′-{2-[4-(2,4- dichloro-phenyl)-1-ethyl- 1H-imidazol-2-yl]-(E)- vinyl}-biphenyl-4-yloxy)- phenyl]- trifluoromethanesulfon- amide |
| 159 | 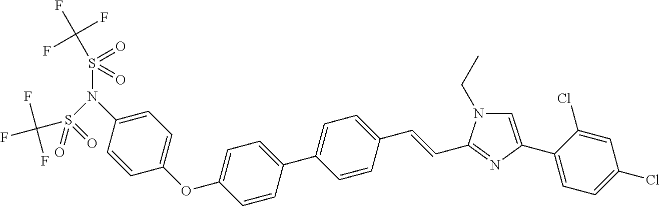 | N-[4-(4′-{2-[4-(2,4- dichloro-phenyl)-1-ethyl- 1H-imidazol-2-yl]-(E)- vinyl}-biphenyl-4-yloxy)- phenyl]- bis(trifluoromethane) sulfonimide |
| 160 | 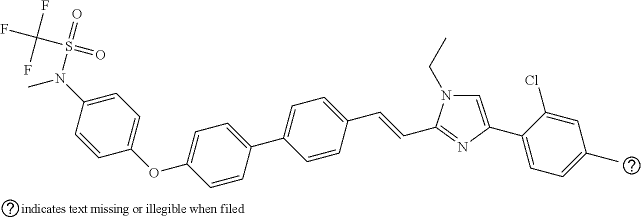 | N-[4-(4′-{2-[4-(2,4- dichloro-phenyl)-1-ethyl- 1H-imidazol-2-yl]-(E)- vinyl}-biphenyl-4-yloxy)- phenyl]-N-methyl- trifluoromethanesulfon- amide |
| 161 | 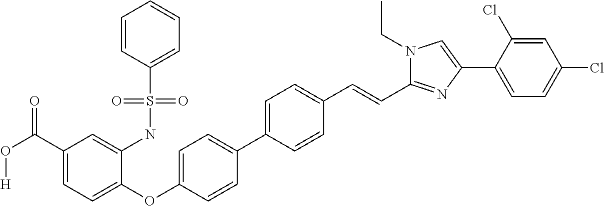 | 3-benzenesulfonylamino- 4-(4′-{2-[4-(2,4-dichloro- phenyl)-1-ethyl-1H- imidazol-2-yl]-(E)-vinyl}- biphenyl-4-yloxy)-benzoic acid |
| 162 | 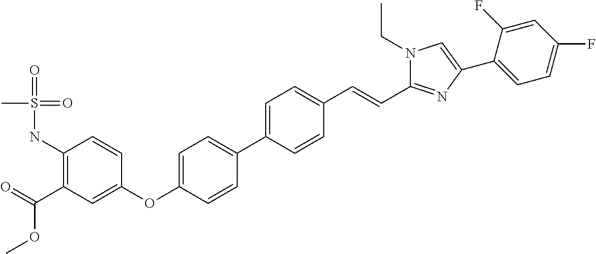 | 5-(4′-{2-[4-(2,4-difluoro- phenyl)-1-ethyl-1H- imidazol-2-yl]-(E)-vinyl}- biphenyl-4-yloxy)-2- methanesulfonylamino- benzoic acid methyl ester |
| 163 | 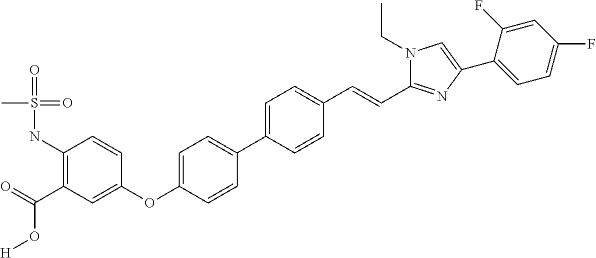 | 5-(4′-{2-[4-(2,4-difluoro- phenyl)-1-ethyl-1H- imidazol-2-yl]-(E)-vinyl}- biphenyl-4-yloxy)-2- methanesulfonylamino- benzoic acid |
| 164 | 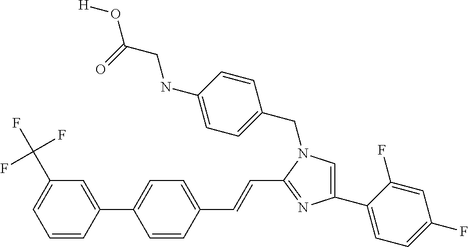 | (4-{4-(2,4-difluoro- phenyl)-2-[2-(3′- trifluoromethyl-biphenyl-4- yl)-(E)-vinyl]-imidazol-1- ylmethyl}-phenylamino)- acetic acid |
| 165 | 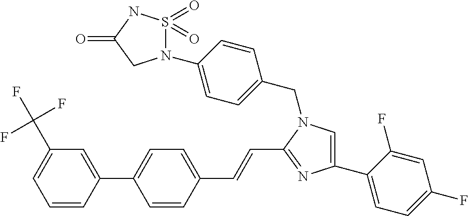 | 5-(4-{4-(2,4-difluoro- phenyl)-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 166 | 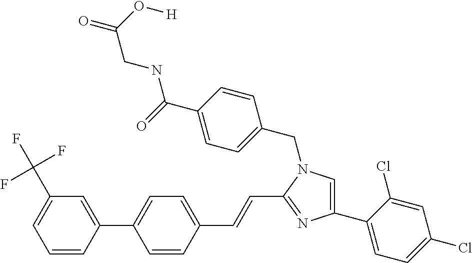 | (4-{4-(2,4-dichloro- phenyl)-2-[2-(3′- trifluoromethyl-biphenyl-4- yl)-(E)-vinyl]-imidazol-1- ylmethyl}-benzoylamino)- acetic acid |
| 167 | 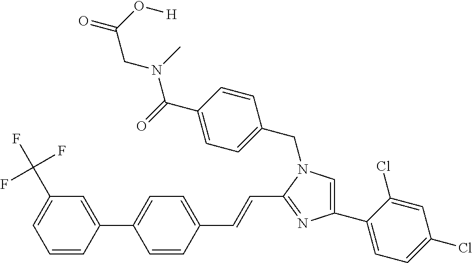 | [(4-{4-(2,4-dichloro- phenyl)-2-[2-(3′- trifluoromethyl-biphenyl-4- yl)-(E)-vinyl]-imidazol-1- ylmethyl}-benzoyl)- methyl-amino]-acetic acid |
| 168 | 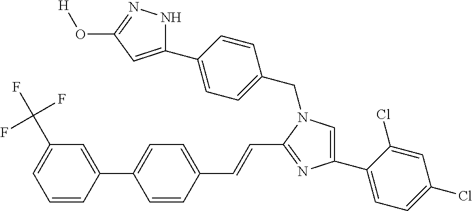 | 5-(4-{4-(2,4-dichloro- phenyl)-2-[2-(3′- trifluoromethyl-biphenyl-4- yl)-(E)-vinyl]-imidazol-1- ylmethyl}-phenyl)-1H- pyrazol-3-ol |
| 169 | 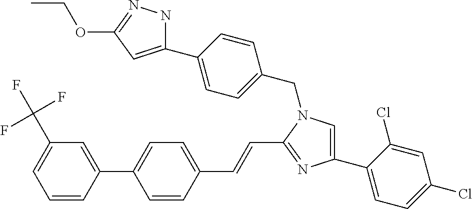 | 5-(4-{4-(2,4-dichloro- phenyl)-2-[2-(3′- trifluoromethyl-biphenyl-4- yl)-(E)-vinyl]-imidazol-1- ylmethyl}-phenyl)-3- ethoxy-1H-pyrazole |
| 170 | 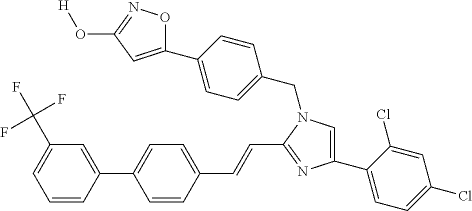 | 5-(4-{4-(2,4-dichloro- phenyl)-2-[2-(3′- trifluoromethyl-biphenyl-4- yl)-(E)-vinyl]-imidazol-1- ylmethyl}-phenyl)- isoxazol-3-ol |
| 171 | 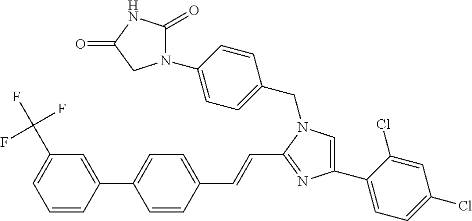 | 1-(4-{4-(2,4-dichloro- phenyl)-2-[2-(3′- trifluoromethyl-biphenyl-4- yl)-(E)-vinyl]-imidazol-1- ylmethyl}-phenyl)- imidazolidine-2,4-dione |
| 172 | 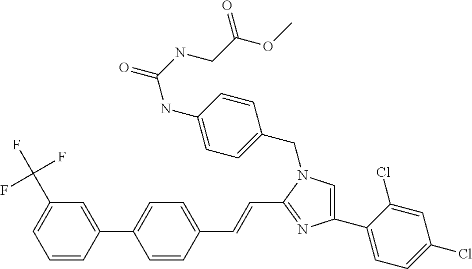 | [3-(4-{4-(2,4-dichloro- phenyl)-2-[2-(3′- trifluoromethyl-biphenyl-4- yl)-(E)-vinyl]-imidazol-1- ylmethyl}-phenyl)-ureido]- acetic acid methyl ester |
| 173 | 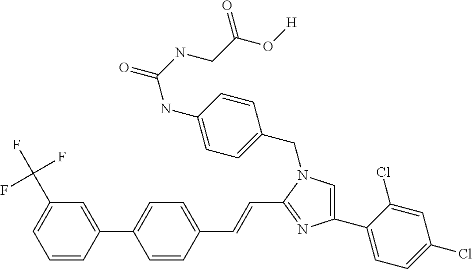 | [3-(4-{4-(2,4-dichloro- phenyl)-2-[2-(3′- trifluoromethyl-biphenyl-4- yl)-(E)-vinyl]-imidazol-1- ylmethyl}-phenyl)-ureido]- acetic acid |
| 174 | 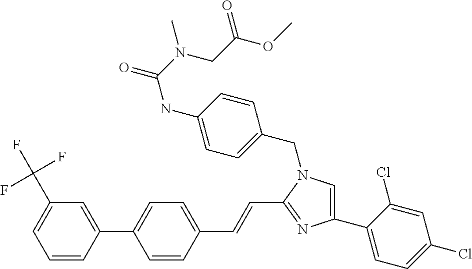 | [3-(4-{4-(2,4-dichloro- phenyl)-2-[2-(3′- trifluoromethyl-biphenyl-4- yl)-(E)-vinyl]-imidazol-1- ylmethyl}-phenyl)-1- methyl-ureido]-acetic acid methyl ester |
| 175 | 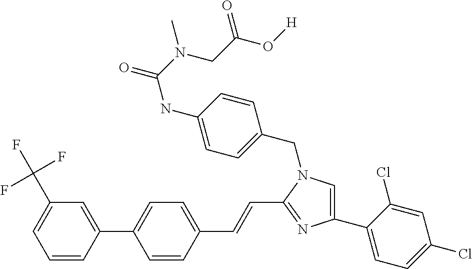 | [3-(4-{4-(2,4-dichloro- phenyl)-2-[2-(3′- trifluoromethyl-biphenyl-4- yl)-(E)-vinyl]-imidazol-1- ylmethyl}-phenyl)-1- methyl-ureido]-acetic acid |
| 176 | 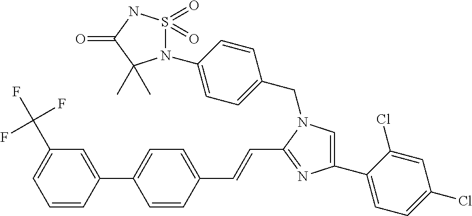 | 5-(4-{4-(2,4-dichloro- phenyl)-2-[2-(3′- trifluoromethyl-biphenyl-4- yl)-(E)-vinyl]-imidazol-1- ylmethyl}-phenyl)-4,4- dimethyl-1,2,5- thiadiazolidine-3-one-1,1- dioxide |
| 177 | 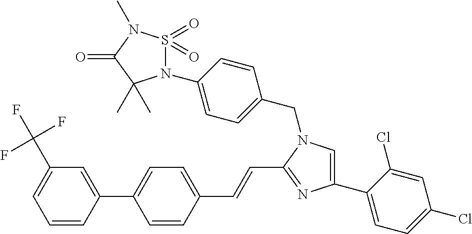 | 5-(4-{4-(2,4-dichloro- phenyl)-2-[2-(3′- trifluoromethyl-biphenyl-4- yl)-(E)-vinyl]-imidazol-1- ylmethyl}-phenyl)-2,4,4- trimethyl-1,2,5- thiadiazolidine-3-one-1,1- dioxide |
| 178 | 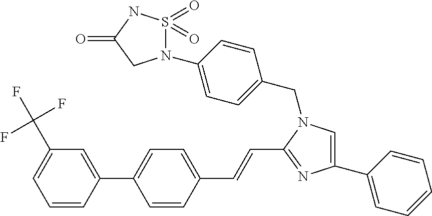 | 5-(4-{4-phenyl-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 179 | 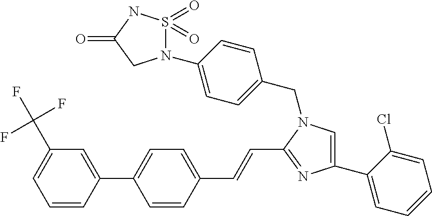 | 5-(4-{4-(2-chloro-phenyl)- 2-[2-(3′-trifluoromethyl- biphenyl-4-yl)-(E)-vinyl]- imidazol-1-ylmethyl}- phenyl)-1,2,5- thiadiazolidine-3-one-1,1- dioxide |
| 180 | 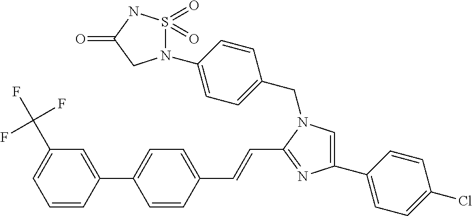 | 5-(4-{4-(4-chloro-phenyl)- 2-[2-(3′-trifluoromethyl- biphenyl-4-yl)-(E)-vinyl]- imidazol-1-ylmethyl}- phenyl)-1,2,5- thiadiazolidine-3-one-1,1- dioxide |
| 181 | 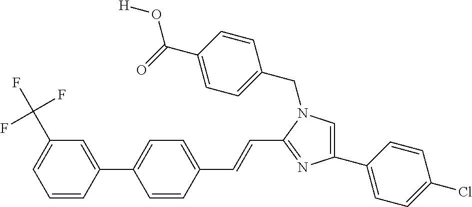 | 4-{4-(4-chloro-phenyl)-2- [2-(3′-trifluoromethyl- biphenyl-4-yl)-(E)-vinyl]- imidazol-1-ylmethyl}- benzoic acid |
| 182 | 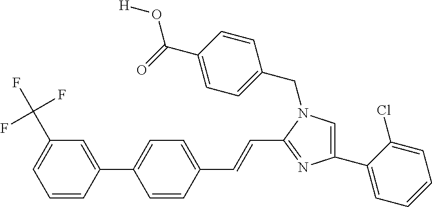 | 4-{4-(2-chloro-phenyl)-2- [2-(3′-trifluoromethyl- biphenyl-4-yl)-(E)-vinyl]- imidazol-1-ylmethyl}- benzoic acid |
| 183 | 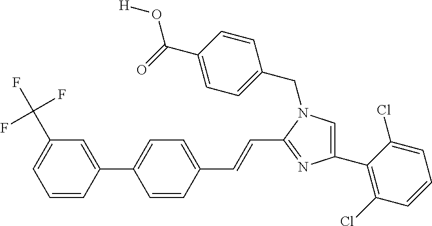 | 4-{4-(2,6-dichloro-phenyl)- 2-[2-(3′-trifluoromethyl- biphenyl-4-yl)-(E)-vinyl]- imidazol-1-ylmethyl}- benzoic acid |
| 184 | 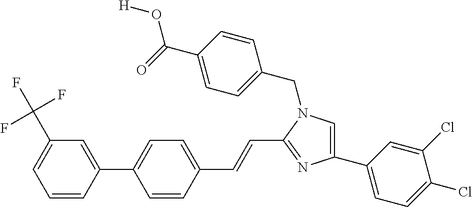 | 4-{4-(3,4-dichloro-phenyl)- 2-[2-(3′-trifluoromethyl- biphenyl-4-yl)-(E)-vinyl]- imidazol-1-ylmethyl}- benzoic acid |
| 185 | 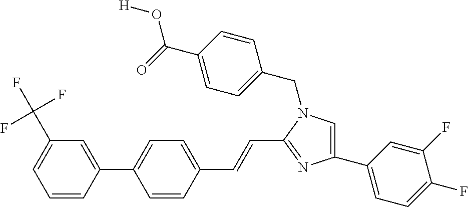 | 4-{4-(3,4-difluoro-phenyl)- 2-[2-(3′-trifluoromethyl- biphenyl-4-yl)-(E)-vinyl]- imidazol-1-ylmethyl}- benzoic acid |
| 186 | 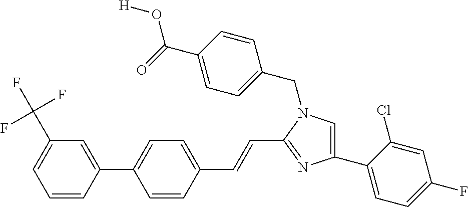 | 4-{4-(2-chloro-4-fluoro- phenyl)-2-[2-(3′- trifluoromethyl-biphenyl-4- yl)-(E)-vinyl]-imidazol-1- ylmethyl}-benzoic acid |
| 187 | 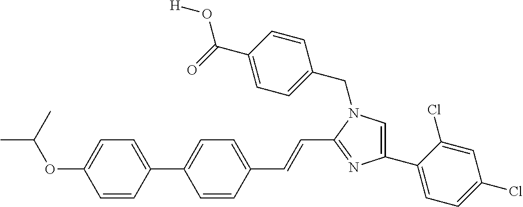 | 4-{4-(2,4-dichloro-phenyl)- 2-[2-(4′-isopropoxy- biphenyl-4-yl)-(E)-vinyl]- imidazol-1-ylmethyl}- benzoic acid |
| 188 | 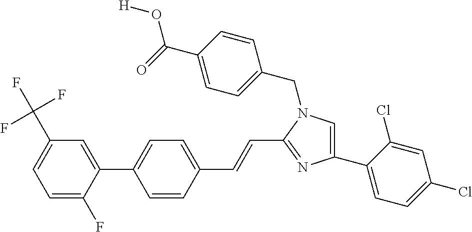 | 4-{4-(2,4-dichloro-phenyl)- 2-[2-(2′-fluoro-5′- trifluoromethyl-biphenyl-4- yl)-(E)-vinyl]-imidazol-1- ylmethyl}-benzoic acid |
| 189 | 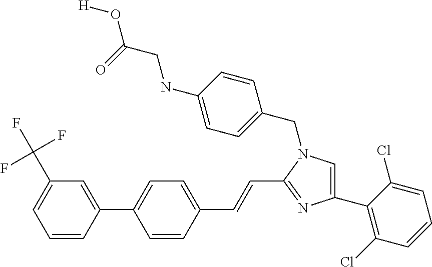 | (4-{4-(2,6-dichloro- phenyl)-2-[2-(3′- trifluoromethyl-biphenyl-4- yl)-(E)-vinyl]-imidazol-1- ylmethyl}-phenylamino)- acetic acid |
| 190 | 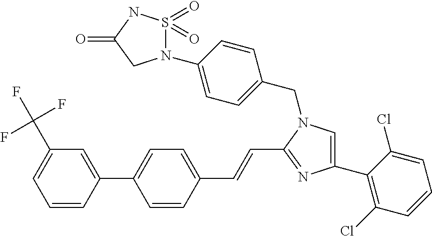 | 5-(4-{4-(2,6-dichloro- phenyl)-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 191 | 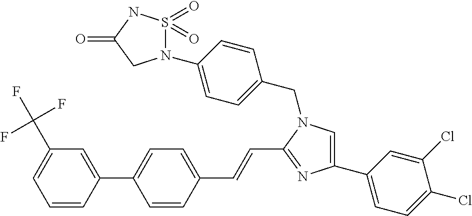 | 5-(4-{4-(3,4-dichloro- phenyl)-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 192 | 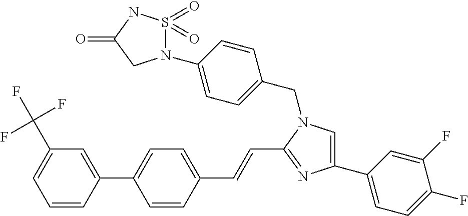 | 5-(4-{4-(3,4-difluoro- phenyl)-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 193 | 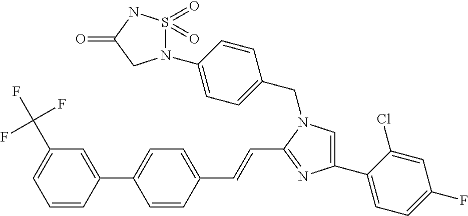 | 5-(4-{4-(2-chloro-4-fluoro- phenyl)-2-[2-(3′- trifluoromethyl-biphenyl-4- yl)-(E)-vinyl]-imidazol-1- ylmethyl}-phenyl)-1,2,5- thiadiazolidine-3-one-1,1- dioxide |
| 194 | 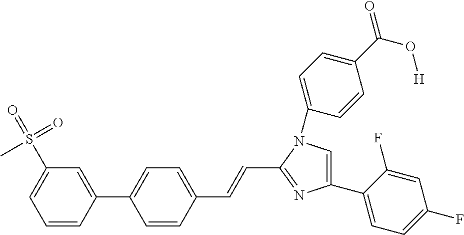 | 4-{4-(2,4-difluoro-phenyl)- 2-[2-(3′-methanesulfonyl- biphenyl-4-yl)-(E)-vinyl]- imidazol-1-yl}-benzoic acid |
| 195 | 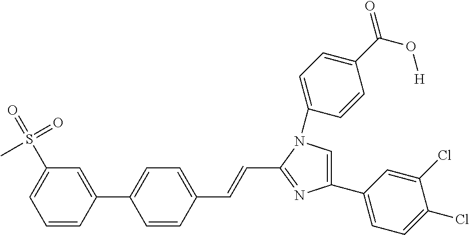 | 4-{4-(3,4-dichloro-phenyl)- 2-[2-(3′-methanesulfonyl- biphenyl-4-yl)-(E)-vinyl]- imidazol-1-yl}-benzoic acid |
| 196 | 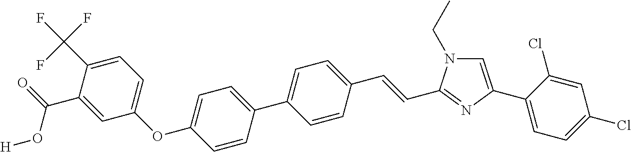 | 5-(4′-{2-[4-(2,4-dichloro- phenyl)-1-ethyl-1H- imidazol-2-yl]-(E)-vinyl}- biphenyl-4-yloxy)-2- trifluoromethyl-benzoic acid |
| 197 | 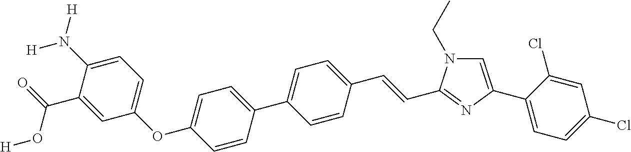 | 2-amino-5-(4′-{2-[4-(2,4- dichloro-phenyl)-1-ethyl- 1H-imidazol-2-yl]-(E)- vinyl}-biphenyl-4-yloxy)- benzoic acid |
| 198 | 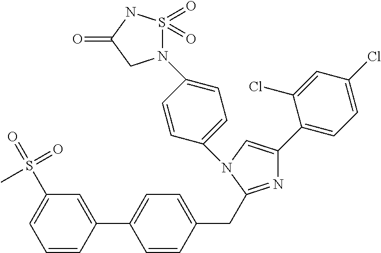 | 5-{4-[4-(2,4-dichloro- phenyl)-2-(3′- methanesulfonyl- biphenyl-4-ylmethyl)- imidazol-1-yl]-phenyl}- 1,2,5-thiadiazolidine-3- one-1,1-dioxide |
| 199 | 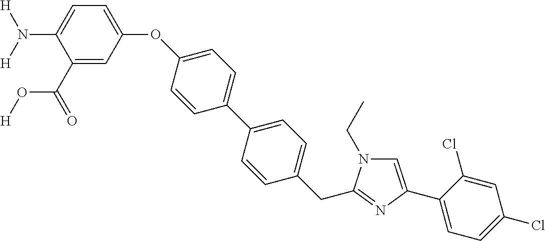 | 2-amino-5-{4′-[4-(2,4- dichloro-phenyl)-1-ethyl- 1H-imidazol-2-ylmethyl]- biphenyl-4-yloxy}-benzoic acid |
| 200 | 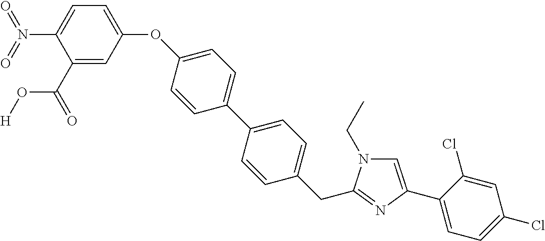 | 5-{4′-[4-(2,4-dichloro- phenyl)-1-ethyl-1H- imidazol-2-ylmethyl]- biphenyl-4-yloxy}-2-nitro- benzoic acid |
| 201 | 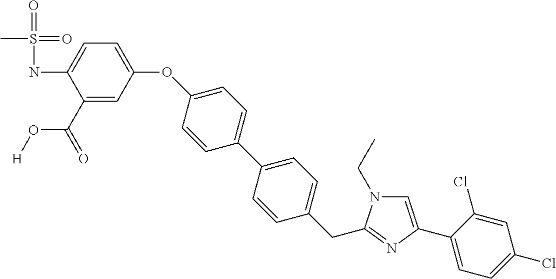 | 5-{4′-[4-(2,4-dichloro- phenyl)-1-ethyl-1H- imidazol-2-ylmethyl]- biphenyl-4-yloxy}-2- methanesulfonylamino- benzoic acid |
| 202 | 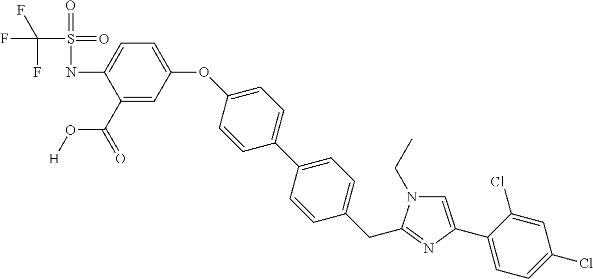 | 5-{4′-[4-(2,4-dichloro- phenyl)-1-ethyl-1H- imidazol-2-ylmethyl]- biphenyl-4-yloxy}-2- trifluoromethanesulfonyl- amino-benzoic acid |
| 203 | 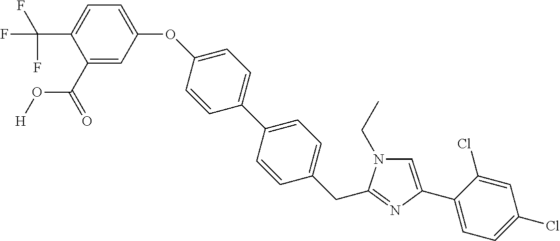 | 5-{4′-[4-(2,4-dichloro- phenyl)-1-ethyl-1H- imidazol-2-ylmethyl]- biphenyl-4-yloxy}-2- trifluoromethyl-benzoic acid |
| 204 | 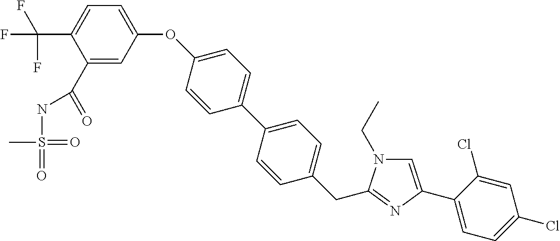 | N-(5-{4′-[4-(2,4-dichloro- phenyl)-1-ethyl-1H- imidazol-2-ylmethyl]- biphenyl-4-yloxy}-2- trifluoromethyl-benzoyl)- methanesulfonamide |
| 205 | 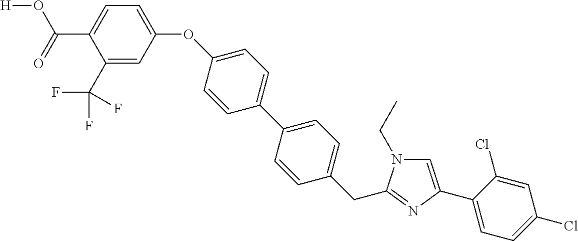 | 4-{4′-[4-(2,4-dichloro- phenyl)-1-ethyl-1H- imidazol-2-ylmethyl]- biphenyl-4-yloxy}-2- trifluoromethyl-benzoic acid |
| 206 | 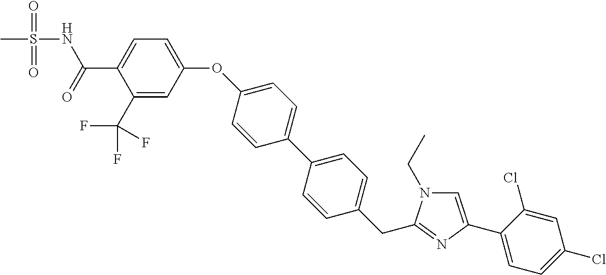 | N-(4-{4′-[4-(2,4-dichloro- phenyl)-1-ethyl-1H- imidazol-2-ylmethyl]- biphenyl-4-yloxy}-2- trifluoromethyl-benzoyl)- methanesulfonamide |
| 207 | 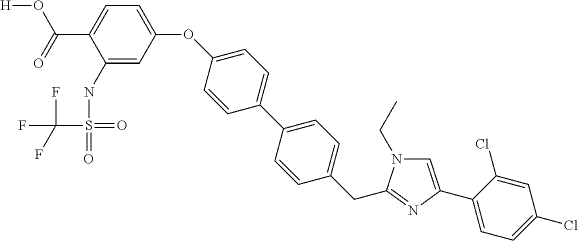 | 4-{4′-[4-(2,4-dichloro- phenyl)-1-ethyl-1H- imidazol-2-ylmethyl]- biphenyl-4-yloxy}-2- trifluoromethanesulfonyl- amino-benzoic acid |
| 208 | 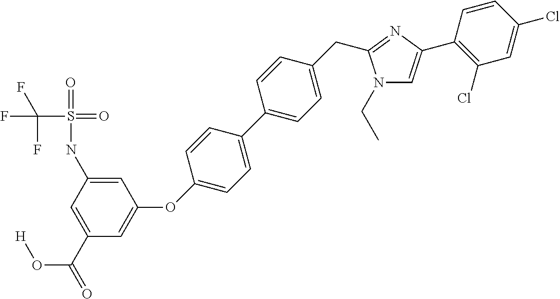 | 3-{4′-[4-(2,4-dichloro- phenyl)-1-ethyl-1H- imidazol-2-ylmethyl]- biphenyl-4-yloxy}-5- trifluoromethanesulfonyl- amino-benzoic acid |
| 209 | 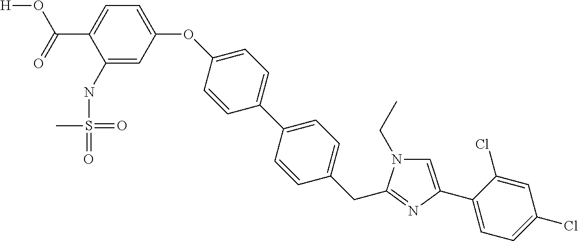 | 4-{4′-[4-(2,4-dichloro- phenyl)-1-ethyl-1H- imidazol-2-ylmethyl]- biphenyl-4-yloxy}-2- methanesulfonylamino- benzoic acid |
| 210 | 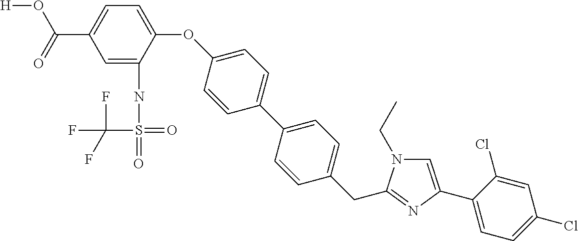 | 4-{4′-[4-(2,4-dichloro- phenyl)-1-ethyl-1H- imidazol-2-ylmethyl]- biphenyl-4-yloxy}-3- trifluoromealnesulfonyl- amino-benzoic acid |
| 211 | 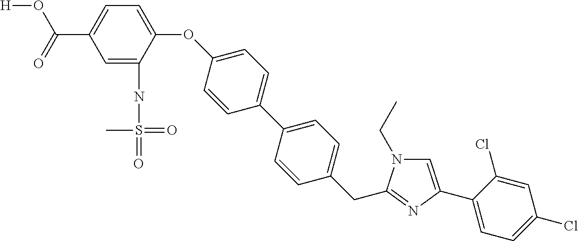 | 4-{4′-[4-(2,4-dichloro- phenyl)-1-ethyl-1H- imidazol-2-ylmethyl]- biphenyl-4-yloxy}-3- methanesulfonylamino- benzoic acid |
| 212 | 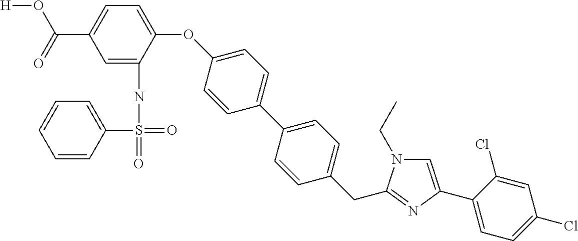 | 3-benzenesulfonylamino- 4-{4′-[4-(2,4-dichloro- phenyl)-1-ethyl-1H- imidazol-2-ylmethyl]- biphenyl-4-yloxy}-benzoic acid |
| 213 | 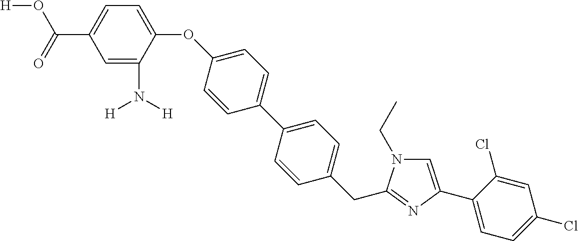 | 3-amino-4-{4′-[4-(2,4- dichloro-phenyl)-1-ethyl- 1H-imidazol-2-ylmethyl]- biphenyl-4-yloxyl-benzoic acid |
| 214 | 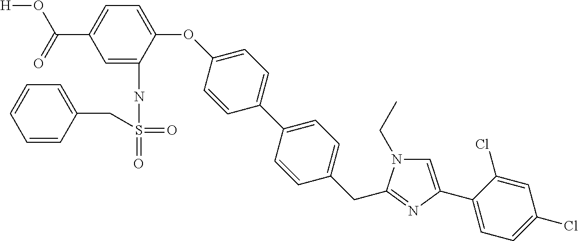 | 4-{4′-[4-(2,4-dichloro- phenyl)-1-ethyl-1H- imidazol-2-ylmethyl]- biphenyl-4-yloxy}-3- phenylmethanesulfonyl- amino-benzoic acid |
| 215 | 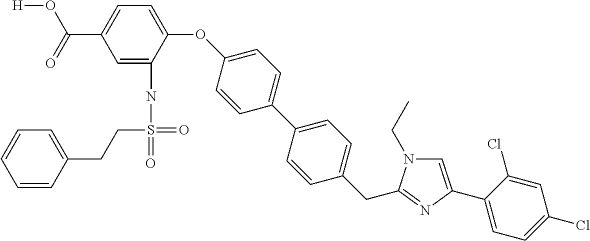 | 4-{4′-[4-(2,4-dichloro- phenyl)-1-ethyl-1H- imidazol-2-ylmethyl]- biphenyl-4-yloxy}-3-(2- phenyl- ethanesulfonylamino)- benzoic acid |
| 216 | 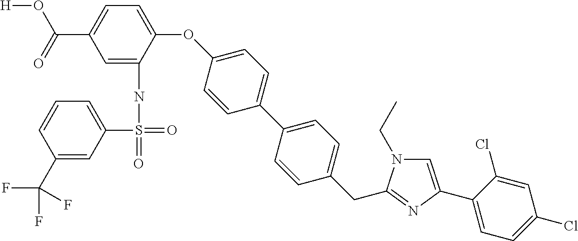 | 4-{4′-[4-(2,4-dichloro- phenyl)-1-ethyl-1H- imidazol-2-ylmethyl]- biphenyl-4-yloxy}-3-(3- trifluoromethyl- benzenesulfonylamino)- benzoic acid |
| 217 | 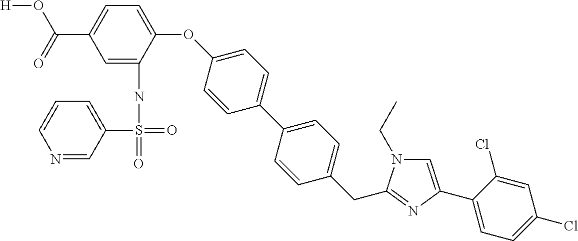 | 4-{4′-[4-(2,4-dichloro- phenyl)-1-ethyl-1H- imidazol-2-ylmethyl]- biphenyl-4-yloxy}-3- (pyridine-3- sulfonylamino)-benzoic acid |
| 218 | 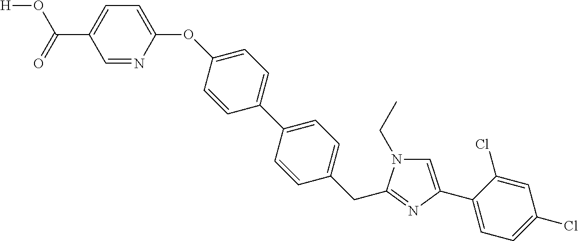 | 6-{4′-[4-(2,4-dichloro- phenyl)-1-ethyl-1H- imidazol-2-ylmethyl]- biphenyl-4-yloxy}-nicotinic acid |
| 219 | 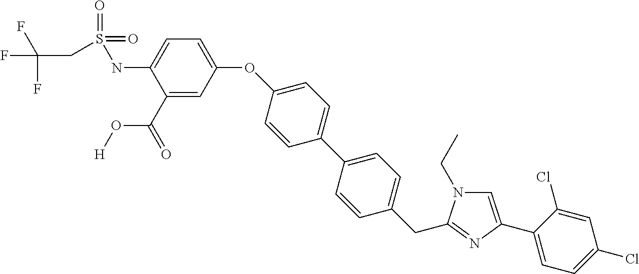 | 5-{4′-[4-(2,4-dichloro- phenyl)-1-ethyl-1H- imidazol-2-ylmethyl]- biphenyl-4-yloxy}-2- (2,2,2-trifluoro- ethanesulfonylamino)- benzoic acid |
| 220 | 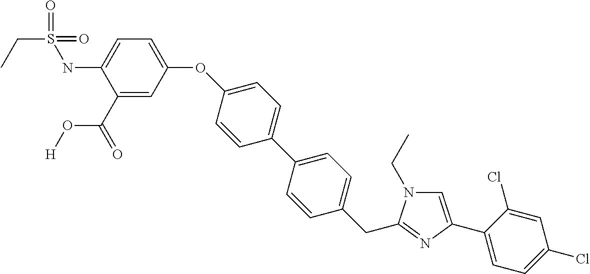 | 5-{4′-[4-(2,4-dichloro- phenyl)-1-ethyl-1H- imidazol-2-ylmethyl]- biphenyl-4-yloxy}-2- ethanesulfonylamino- benzoic acid |
| 221 | 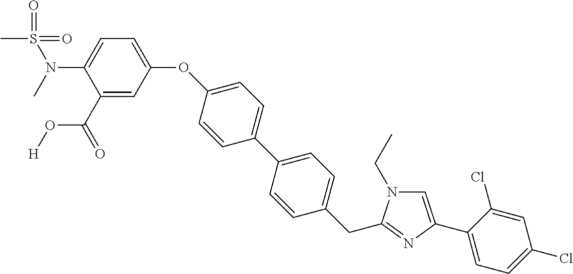 | 5-{4′-[4-(2,4-dichloro- phenyl)-1-ethyl-1H- imidazol-2-ylmethyl]- biphenyl-4-yloxy}-2- (methanesulfonyl-methyl- amino)-benzoic acid |
| 222 | 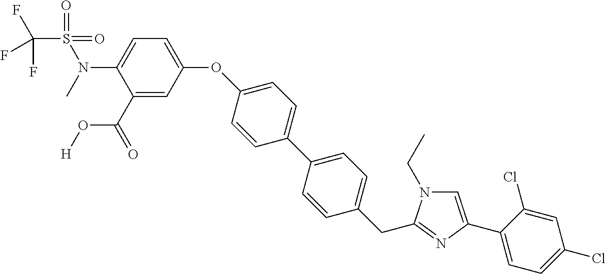 | 5-{4′-[4-(2,4-dichloro- phenyl)-1-ethyl-1H- imidazol-2-ylmethyl]- biphenyl-4-yloxy}-2- (methyl-trifluoromethane sulfonyl-amino)-benzoic acid |
| 223 | 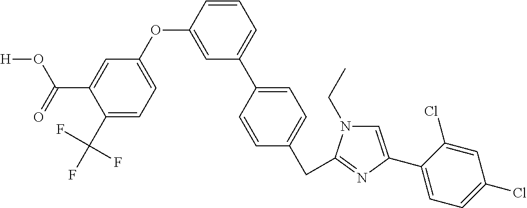 | 5-{4′-[4-(2,4-dichloro- phenyl)-1-ethyl-1H- imidazol-2-ylmethyl]- biphenyl-3-yloxy}-2- trifluoromethyl-benzoic acid |
| 224 | 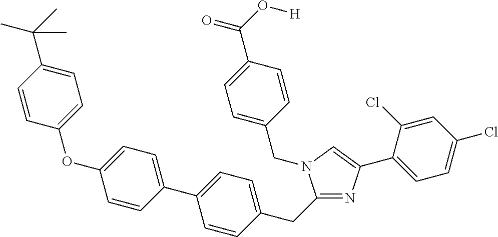 | 4-[2-[4′-(4-tert-Butyl- phenoxy)-biphenyl-4- ylmethyl]-4-(2,4-dichloro- phenyl)-imidazol-1- ylmethyl]-benzoicacid |
| 225 | 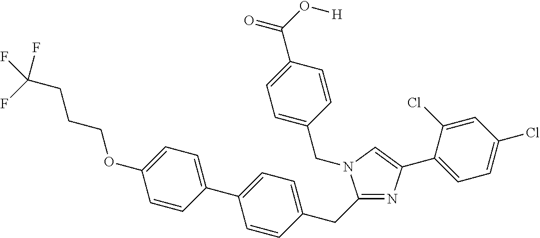 | 4-{4-(2,4-Dichloro- phenyl)-2-[4′-(4,4,4- trifluoro-butoxy)-biphenyl- 4-ylmethyl]-imidazol-1- ylmethyl}-benzoic acid |
| 226 | 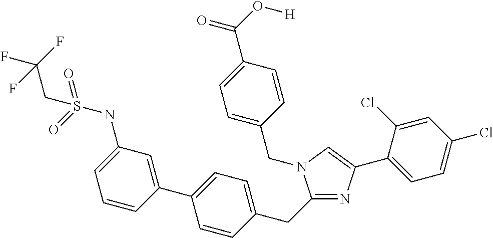 | 4-{4-(2,4-Dichloro- phenyl)-2-[3′-(2,2,2- trifluoro- ethanesulfonylamino)- biphenyl-4-ylmethyl]- imidazol-1-ylmethyl}- benzoic acid |
| 227 | 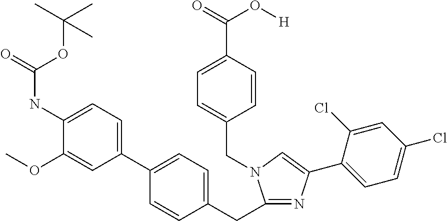 | 4-[2-(4′-tert- Butoxycarbonylamino-3′- methoxy-biphenyl-4- ylmethyl)-4-(2,4-dichloro- phenyl)-imidazol-1- ylmethyl]-benzoic acid |
| 228 | 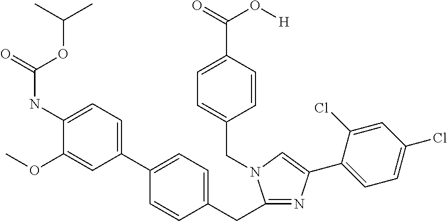 | 4-[4-(2,4-Dichloro- phenyl)-2-(4′- isopropoxycarbonylamino- 3′-methoxy-biphenyl-4- ylmethyl)-imidazol-1- ylmethyl]-benzoic acid |
| 229 | 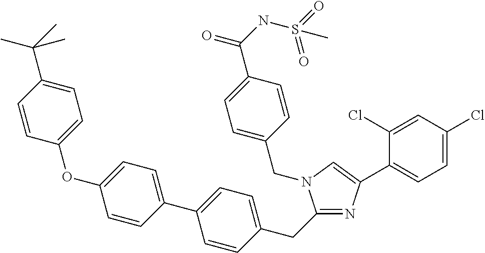 | N-{4-[2-[4′-(4-tert-Butyl- phenoxy)-biphenyl-4- ylmethyl]-4-(2,4-dichloro- phenyl)-imidazol-1- ylmethyl]-benzoyl}- methanesulfonamide |
| 230 | 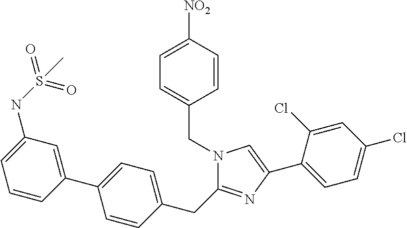 | N-{4′-[1-(4-Nitro-benzyl)- 4-(2,4-dichloro-phenyl)- 1H-imidazol-2-ylmethyl]- biphenyl-3-yl}- methanesulfonamide |
| 231 | 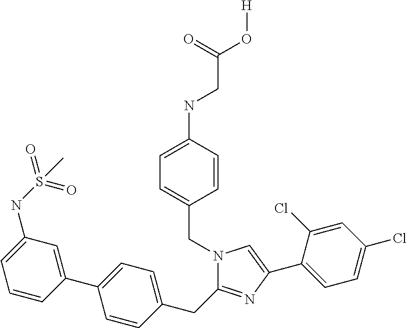 | {4-[4-(2,4-Dichloro- phenyl)-2-(3′- methanesulfonylamino- biphenyl-4-ylmethyl)- imidazol-1-ylmethyl]- phenylamino}-acetic acid |
| 232 | 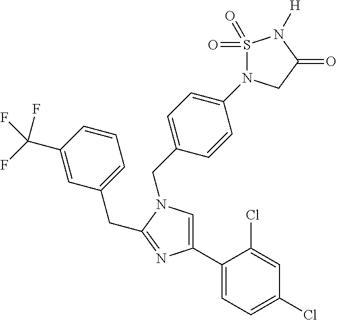 | 5-{4-[4-(2,4-Dichloro- phenyl)-2-(3- trifluoromethyl-benzyl)- imidazol-1-ylmethyl]- phenyl}-1-[1,2,5]- thiadiazolidin-3-one-1,1- dioxide |
| 233 | 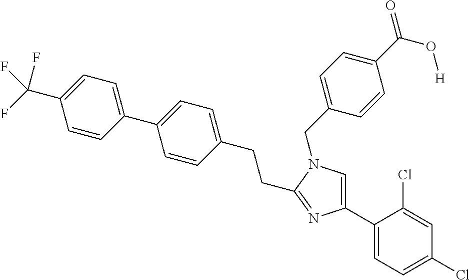 | 4-{4-(2,4-Dichloro- phenyl)-2-[2-(4′- trifluoromethyl-biphenyl-4- yl)-ethyl]-imidazol-1- ylmethyl}-benzoic acid |
| 234 | 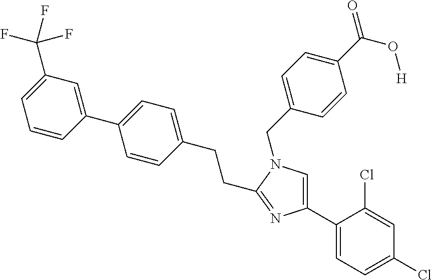 | 4-{4-(2,4-Dichloro- phenyl)-2-[2-(3′- trifluoromethyl-biphenyl-4- yl)-ethyl]-imidazol-1- ylmethyl}-benzoic acid |
| 235 | 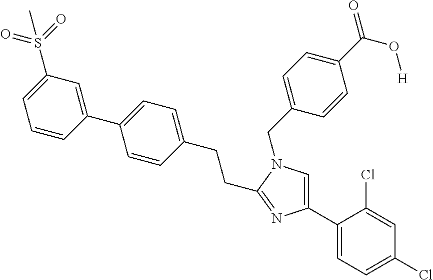 | 4-{4-(2,4-Dichloro- phenyl)-2-[2-(3′- methanesulfonyl- biphenyl-4-yl)-ethyl]- imidazol-1-ylmethyl}- benzoic acid |
| 236 | 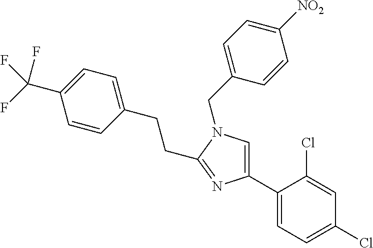 | 4-{4-(2,4-Dichloro- phenyl)-2-[2-(4- trifluoromethyl-phenyl)- ethyl]-nitro benzyl imidazole |
| 237 | 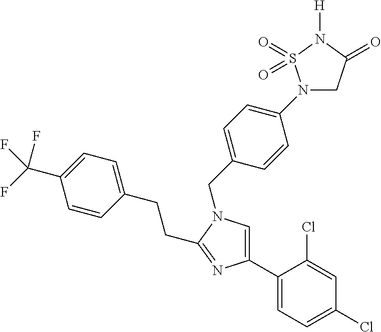 | 5-(4-{4-(2,4-Dichloro- phenyl)-2-[2-(4- trifluoromethyl-phenyl)- ethyl]-imidazol-1- ylmethyl}-phenyl)-1,2,5- thiadiazolidin-3-one 1,1dioxide |
| 238 | 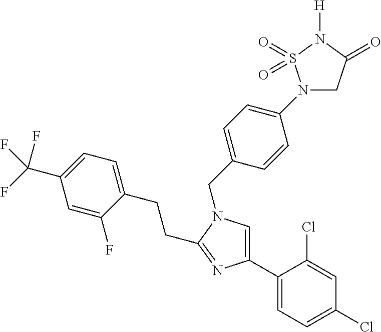 | 5-(4-{4-(2,4-Dichloro- phenyl)-2-[2-(2-fluoro-4- trifluoromethyl-phenyl)- ethyl]-imidazol-1- ylmethyl}-phenyl)-1,2,5- thiadiazolidin-3-one-1,1- dioxide |
| 239 | 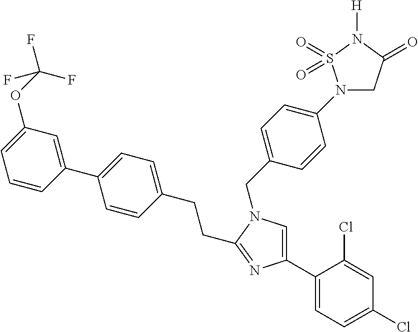 | 5-(4-{4-(2,4-Dichloro- phenyl)-2-[2-(3′- trifluoromethoxy-biphenyl- 4-yl)-ethyl]-imidazol-1- ylmethyl}-phenyl)-1,2,5- thiadiazolidin-3-one-1,1- dioxide |
| 240 | 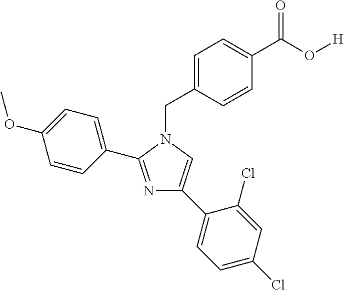 | 4-[4-(2,4-Dichloro- phenyl)-2-(4-methoxy- phenyl)-imidazol-1- ylmethyl]-benzoic acid |
| 241 | 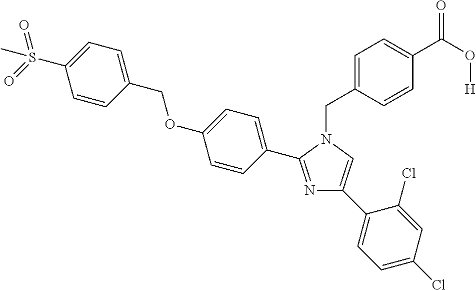 | 4-{4-(2,4-Dichloro- phenyl)-2-[4-(4- methanesulfonyl- benzyloxy)-phenyl]- imidazol-1-ylmethyl}- benzoic acid |
| 242 | 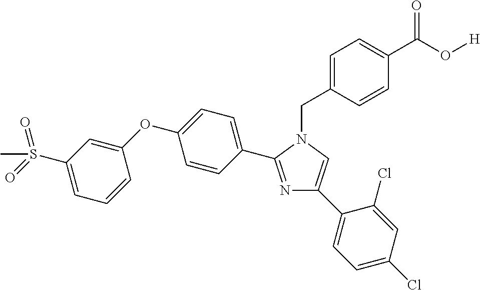 | 4-{4-(2,4-Dichloro- phenyl)-2-[4-(3- methanesulfonyl- phenoxy)-phenyl]- imidazol-1-ylmethyl}- benzoic acid |
| 243 | 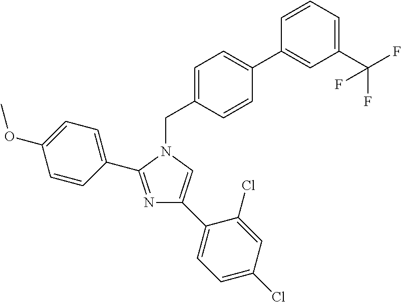 | 4-(2,4-Dichloro-phenyl)-2- (4-methoxy-phenyl)-1-(3′- trifluoromethyl-biphenyl-4- ylmethyl) 1H-imidazole |
| 244 | 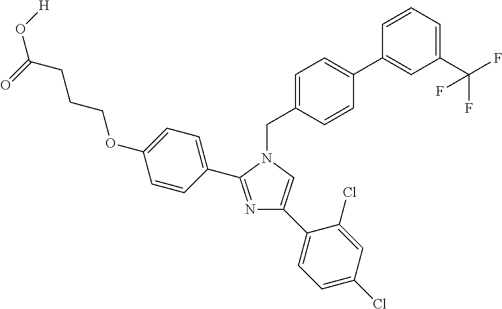 | 4-{4-[4-(2,4-Dichloro- phenyl)-1-(3′- trifluoromethyl-biphenyl-4- ylmethyl)-1H-imidazol-2- yl]-phenoxyl-butyric acid |
| 245 | 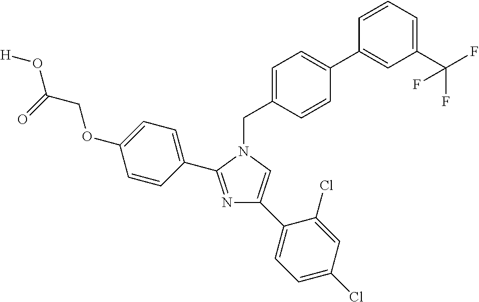 | {4-[4-(2,4-Dichloro- phenyl)-1-(3′- trifluoromethyl-biphenyl-4- ylmethyl)-1H-imidazol-2- yl]-phenoxy}-acetic acid |
| 246 | 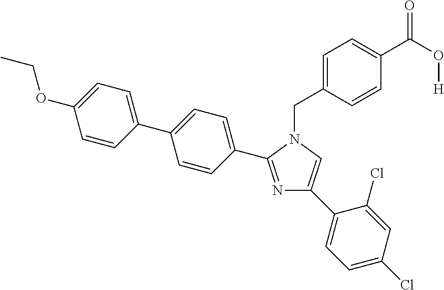 | 4-[4-(2,4-Dichloro- phenyl)-2-(4′-ethoxy- biphenyl-4-yl)-imidazol-1- ylmethyl]-benzoic acid |
| 247 | 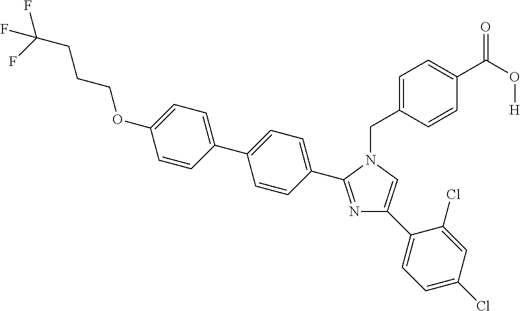 | 4-{4-(2,4-Dichloro- phenyl)-2-[4′-(4,4,4- trifluoro-butoxy)-biphenyl- 4-yl]-imidazol-1-ylmethyl}- benzoic acid |
| 248 | 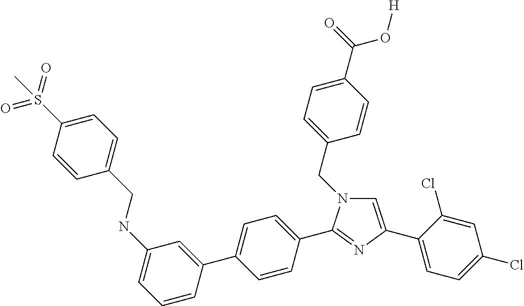 | 4-{4-(2,4-Dichloro- phenyl)-2-[3′-(4- methanesulfonyl- benzylamino)-biphenyl-4- yl]-imidazol-1-ylmethyl}- benzoic acid |
| 249 | 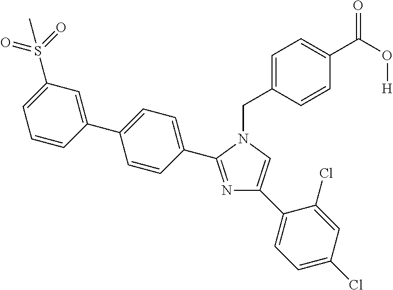 | 4-[4-(2,4-Dichloro- phenyl)-2-(3′- methanesulfonyl- biphenyl-4-yl)-imidazol-1- ylmethyl]-benzoic acid |
| 250 | 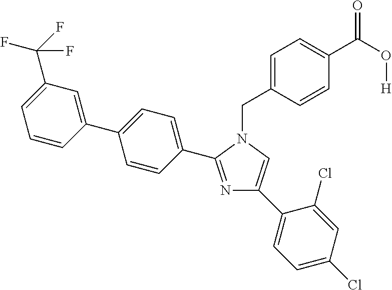 | 4-[4-(2,4-Dichloro- phenyl)-2-(3′- trifluoromethyl-biphenyl-4- yl)-imidazol-1-ylmethyl]- benzoic acid |
| 251 | 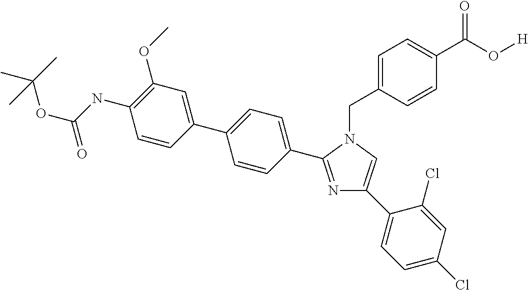 | 4-[2-(4′-tert- Butoxycarbonylamino-3′- methoxy-biphenyl-4-yl)-4- (2,4-dichloro-phenyl)- imidazol-1-ylmethyl]- benzoic acid |
| 252 | 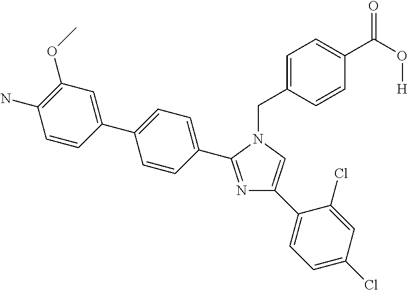 | 4-[2-(4′-Amino-3′- methoxy-biphenyl-4-yl)-4- (2,4-dichloro-phenyl)- imidazol-1-ylmethyl]- benzoic acid |
| 253 | 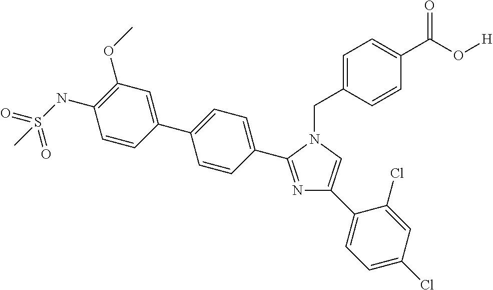 | 4-[4-(2,4-Dichloro- phenyl)-2-(4′- methanesulfonylamino-3′- methoxy-biphenyl-4-yl)- imidazol-1-ylmethyl]- benzoic acid |
| 254 | 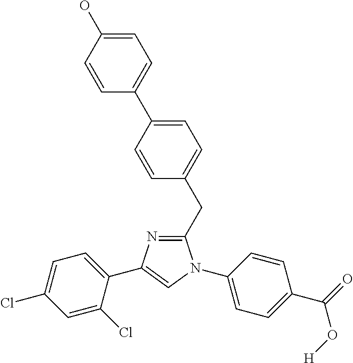 | 4-[4-(2,4-Dichloro- phenyl)-2-(4′-hydroxy- biphenyl-4-ylmethyl)- imidazol-1-yl]-benzoic acid |
| 255 | 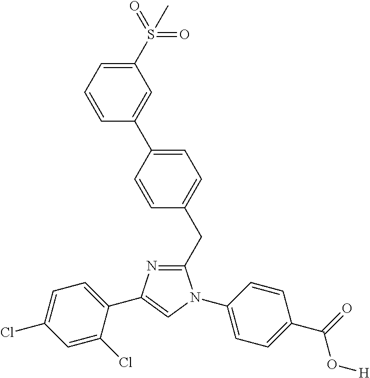 | 4-[4-(2,4-Dichloro- phenyl)-2-(3′- methanesulfonyl- biphenyl-4-ylmethyl)- imidazol-1-yl]-benzoic acid |
| 256 | 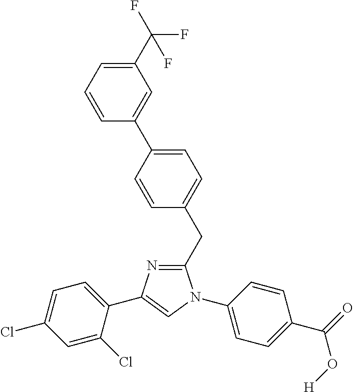 | 4-[4-(2,4-Dichloro- phenyl)-2-(3′- trifluoromethy-biphenyl-4- ylmethyl)-imidazol-1-yl]- benzoic acid |
| 257 | 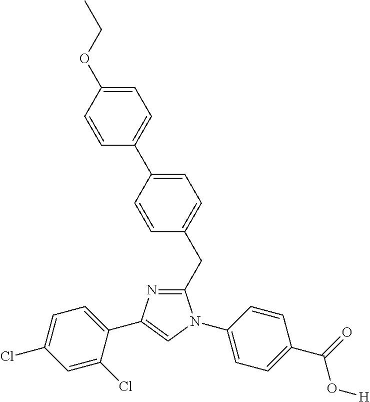 | 4-[4-(2,4-Dichloro- phenyl)-2-(4′-ethoxy- biphenyl-4-ylmethyl)- imidazol-1-yl]-benzoic acid |
| 258 | 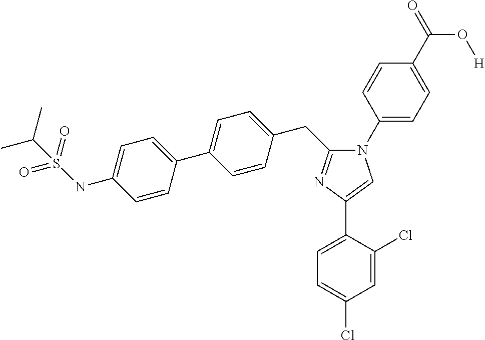 | 4-{4-(2,4-Dichloro- phenyl)-2-[4′-(propane-2- sulfonylamino)-biphenyl- 4-ylmethyl]-imidazol-1-yl}- benzoic acid |
| 259 | 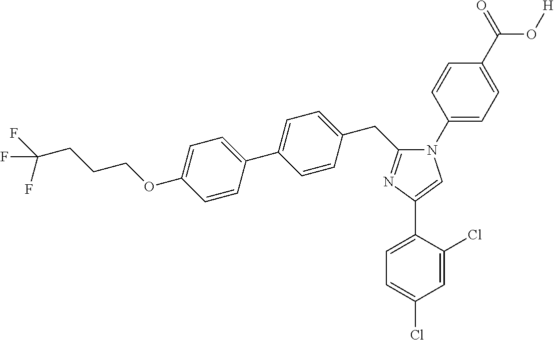 | 4-{4-(2,4-Dichloro- phenyl)-2-[4′-(4,4,4- trifluoro-butoxy)-biphenyl- 4-ylmethyl]-imidazol-1-yl}- benzoic acid |
| 260 | 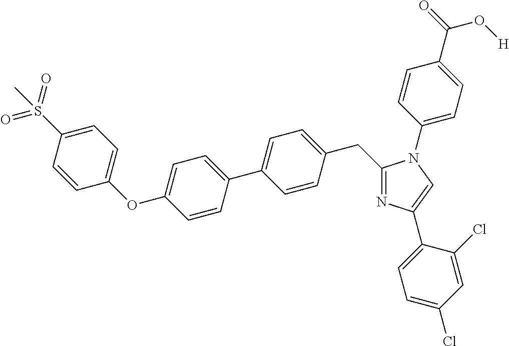 | 4-{4-(2,4-Dichloro- phenyl)-2-[4′-(4- methanesulfonyl- phenoxy)-biphenyl-4- ylmethyl]-imidazol-1-yl}- benzoic acid |
| 261 | 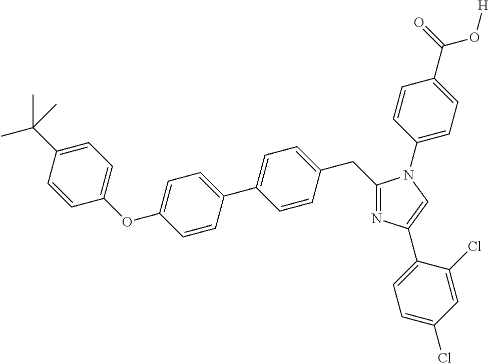 | 4-[2-[4′-(4-tert-Butyl- phenoxy)-biphenyl-4- ylmethyl]-4-(2,4-dichloro- phenyl)-imidazol-1-yl]- benzoic acid |
| 262 | 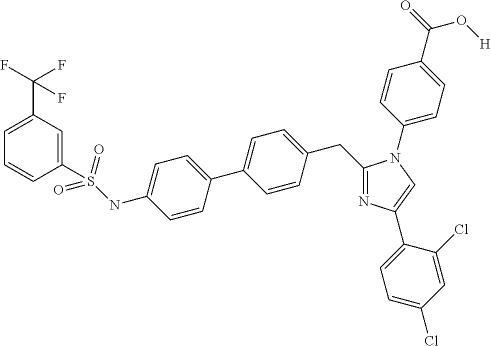 | 4-{4-(2,4-Dichloro- phenyl)-2-[4′-(3- trifluoromethyl- benzenesulfonylamino)- biphenyl-4-ylmethyl]- imidazol-1-yl}-benzoic acid |
| 263 | 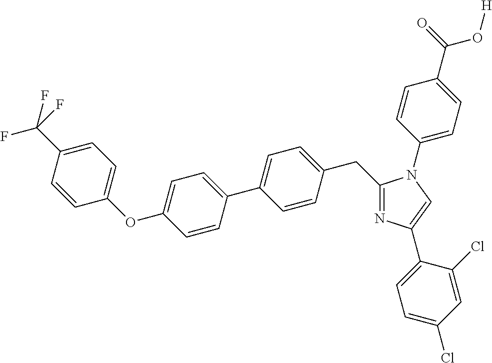 | 4-{4-(2,4-Dichloro- phenyl)-2-[4′-(4- trifluoromethyl-phenoxy)- biphenyl-4-yl- methyl]imidazol-yl}- benzoic acid |
| 264 | 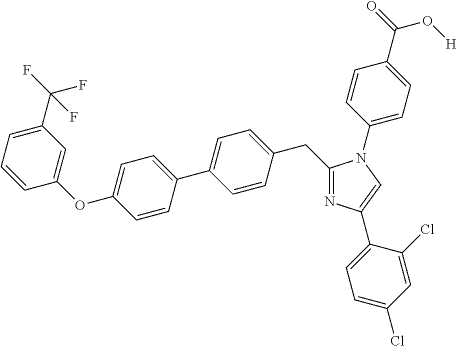 | 4-{4-(2,4-Dichloro- phenyl)-2-[4′-(3- trifluoromethyl-phenoxy)- biphenyl-4-ylmethyl]- imidazol-1-yl}-benzoic acid |
| 265 | 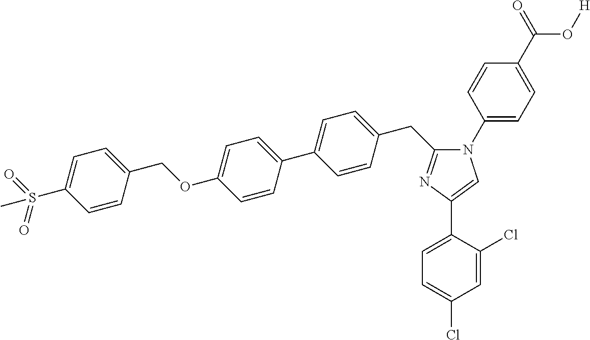 | 4-{4-(2,4-Dichloro- phenyl)-2-[4′-(4- methanesulfonyl- benzyloxy)-biphenyl-4- ylmethyl]-imidazol-1-yl}- benzoic acid |
| 266 | 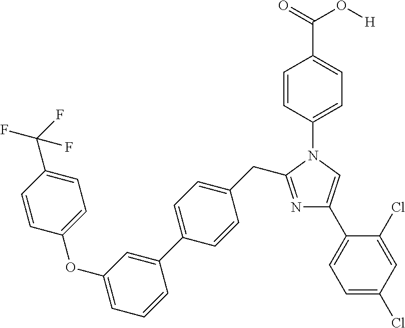 | 4-{4-(2,4-Dichloro- phenyl)-2-[3′-(4- trifluoromethyl-phenoxy)- biphenyl-4-ylmethyl]- imidazol-1-yl}-benzoic acid |
| 267 | 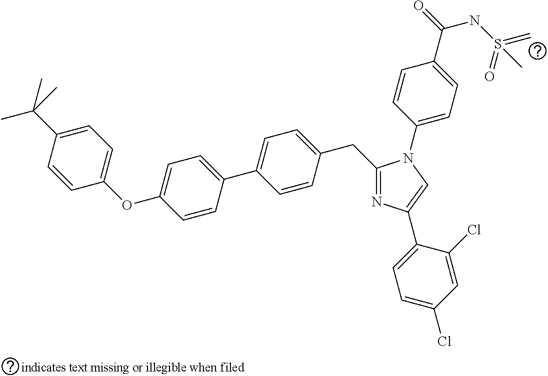 | N-{4-[2-[4′-(4-tert-Butyl- phenoxy)-biphenyl-4- ylmethyl]-4-(2,4-dichloro- phenyl)-imidazol-1-yl]- benzoyl}- methanesulfonamide |
| 268 | 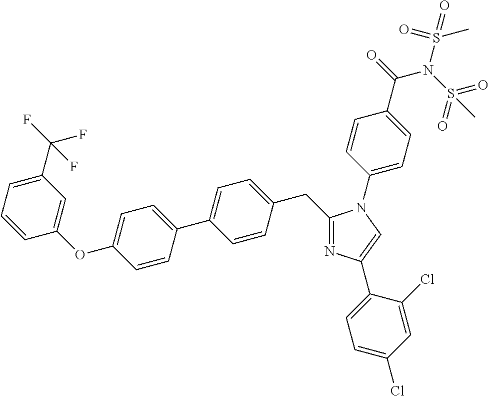 | N-(4-{4-(2,4-Dichloro- phenyl)-2-[4′-(3- trifluoromethyl-phenoxy)- biphenyl-4-ylmethyl]- imidazol-1-yl}-benzoyl)-N- N-dimethanesulfonamide |
| 269 | 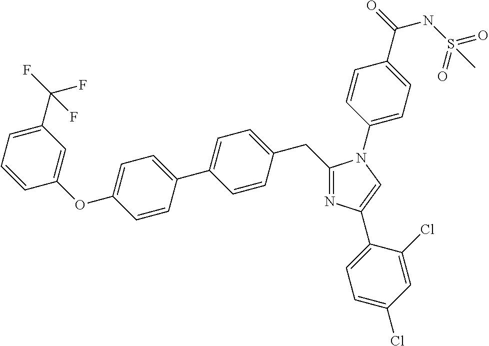 | N-(4-{4-(2,4-Dichloro- phenyl)-2-[4′-(3- trifluoromethyl-phenoxy)- biphenyl-4-ylmethyl]- imidazol-1-yl}-benzoyl)- methanesulfonamide |
| 270 | 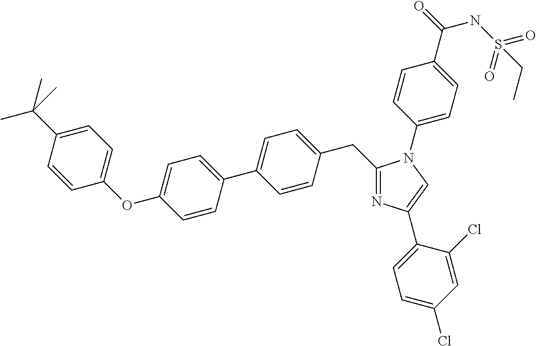 | Ethanesulfonic acid 4-[2- [4′-(4-tert-butyl-phenoxy)- biphenyl-4-ylmethyl]-4- (2,4-dichloro-phenyl)- imidazol-1-yl]- benzoylamide |
| 271 | 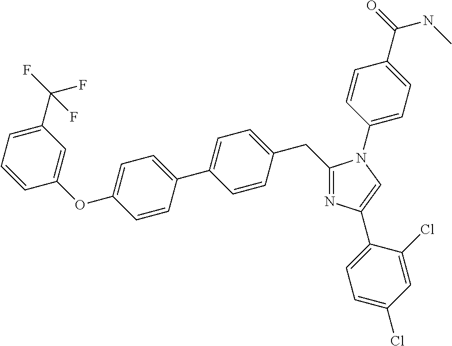 | 4-{4-(2,4-Dichloro- phenyl)-2-[4′-(3- trifluoromethyl-phenoxy)- biphenyl-4-ylmethyl]- imidazol-1-yl}-N-methyl- benzamide |
| 272 | 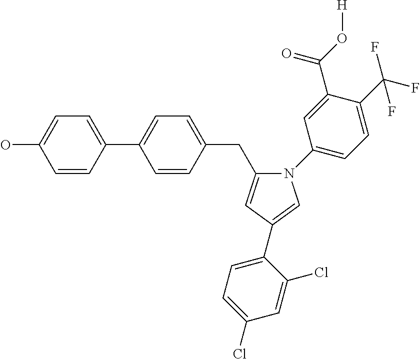 | 5-[4-(2,4-Dichloro- phenyl)-2-(4′-hydroxy- biphenyl-4-ylmethyl)- imidazol-1-yl]-2- trifluoromethyl-benzoic acid |
| 273 | 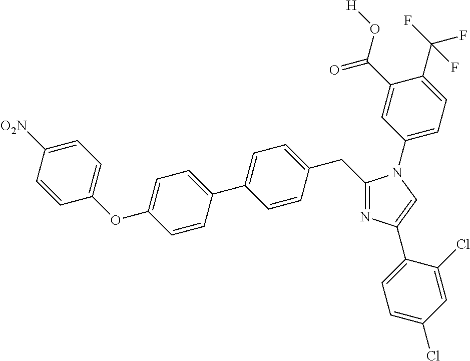 | 5-[2-[4′-(4-Nitro-phenoxy)- biphenyl-4-ylmethyl]-4- (2,4-dichloro-phenyl)- imidazol-1-yl]-2- trifluoromethyl-benzoic acid |
| 274 | 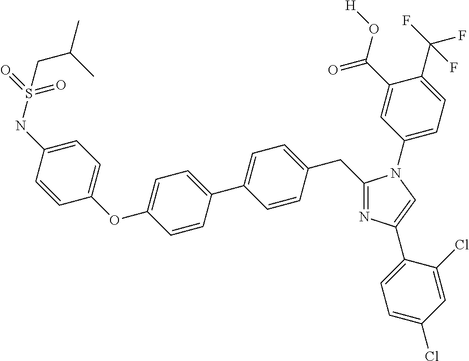 | 5-(4-(2,4-Dichloro- phenyl)-2-{4′-[4-(2-methyl- propane-1- sulfonylamino)-phenoxy]- biphenyl-4-ylmethyl}- imidazol-1-yl)-2- trifluoromethyl-benzoic acid |
| 275 | 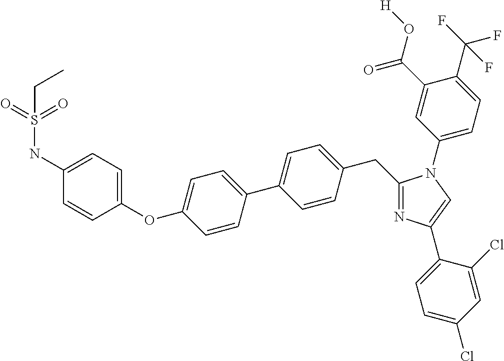 | 5-{4-(2,4-Dichloro- phenyl)-2-[4′-(4- ethanesulfonylamino- phenoxy)-biphenyl-4- ylmethyl]-imidazol-1-yl}-2- trifluoromethyl-benzoic acid |
| 276 | 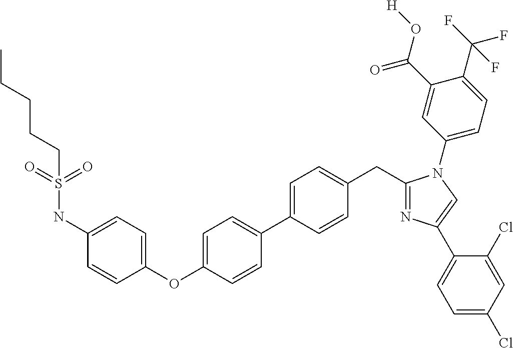 | 5-(4-(2,4-Dichloro- phenyl)-2-{4′-[4-(pentane- 1-sulfonylamino)- phenoxy]-biphenyl-4- ylmethyl}-imidazol-1-yl)-2- trifluoromethyl-benzoic acid |
| 277 | 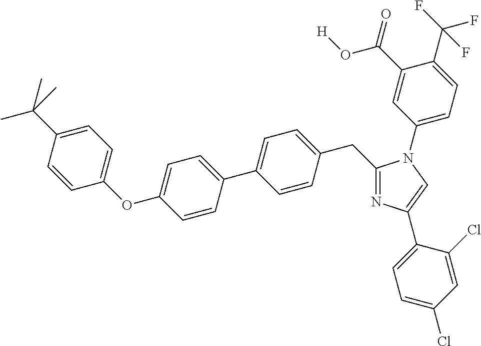 | 5-[2-[4′-(4-tert-Butyl- phenoxy)-biphenyl-4- ylmethyl]-4-(2,4-dichloro- phenyl)-imidazol-1-yl]-2- trifluoromethyl-benzoic acid |
| 278 | 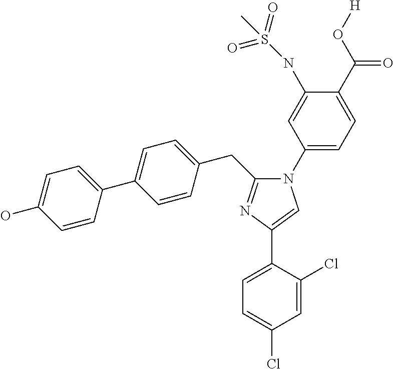 | 4-[4-(2,4-Dichloro- phenyl)-2-(4′-hydroxy- biphenyl-4-ylmethyl)- imidazol-1-yl]-2- methanesulfonylamino- benzoic acid |
| 279 | 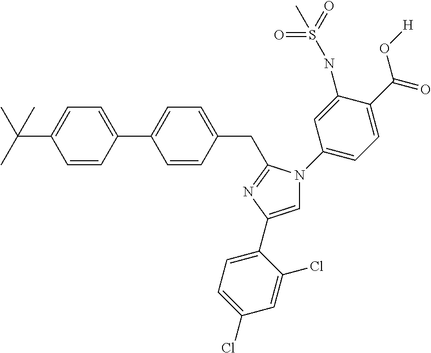 | 4-[2-(4′-tert-Butyl- biphenyl-4-ylmethyl)-4- (2,4-dichloro-phenyl)- imidazol-1-yl]-2- methanesulfonylamino- benzoic acid |
| 280 | 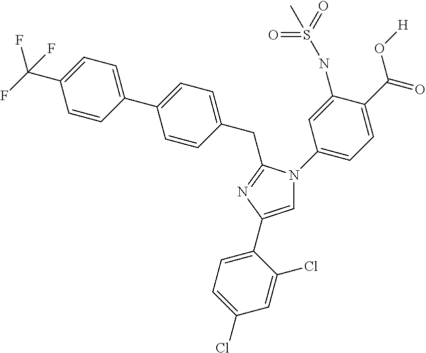 | 4-[4-(2,4-Dichloro- phenyl)-2-(4′- trifluoromethyl-biphenyl-4- ylmethyl)-imidazol-1-yl]-2- methanesulfonylamino- benzoic acid |
| 281 | 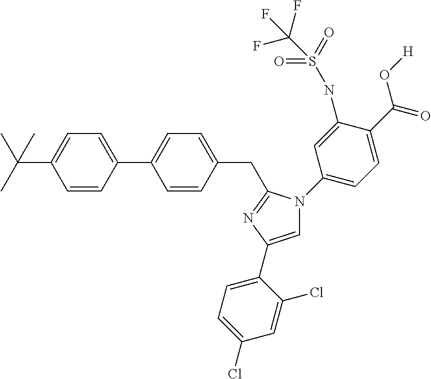 | 4-[2-(4′-tert-Butyl- biphenyl-4-ylmethyl)-4- (2,4-dichloro-phenyl)- imidazol-1-yl]-2- trifluoromethanesulfonyl- amino-benzoic acid |
| 282 | 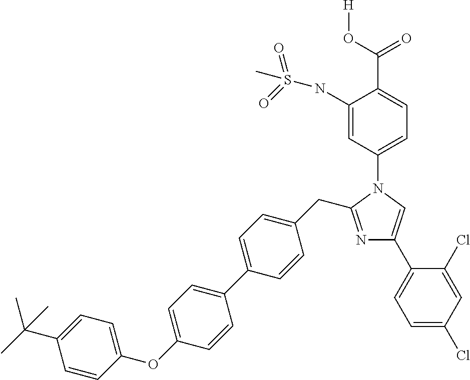 | 4-[2-[4′-(4-tert-Butyl- phenoxy)-biphenyl-4- ylmethyl]-4-(2,4-dichloro- phenyl)-imidazol-1-yl]-2- methanesulfonylamino- benzoic acid |
| 283 | 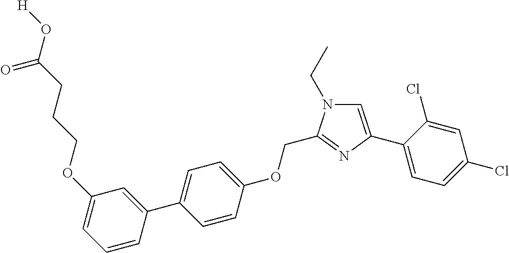 | 4-{4′-[4-(2,4-Dichloro- phenyl)-1-ethyl-1H- imidazol-2-ylmethoxy]- biphenyl-3-yloxy}-butyric acid |
| 284 | 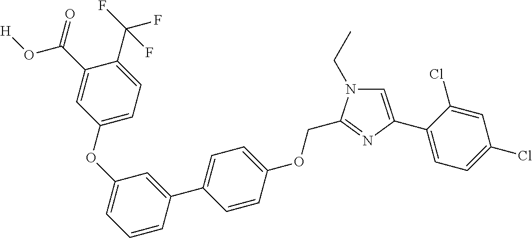 | 5-{4′-[4-(2,4-Dichloro- phenyl)-1-ethyl-1H- imidazol-2-ylmethoxy]- biphenyl-3-yloxy}-2- trifluoromethyl-benzoic acid |
| 285 | 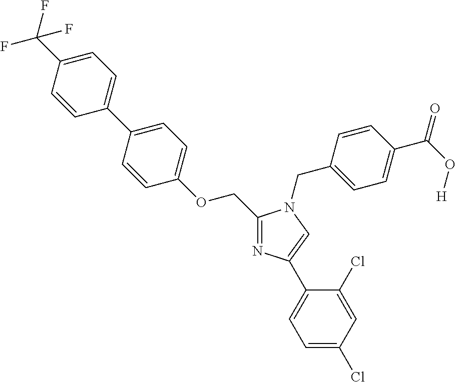 | 4-[4-(2,4-Dichloro- phenyl)-2-(4′- trifluoromethyl-biphenyl- 4-yloxymethyl)-imidazol- 1-ylmethyl]-benzoic acid |
| 286 | 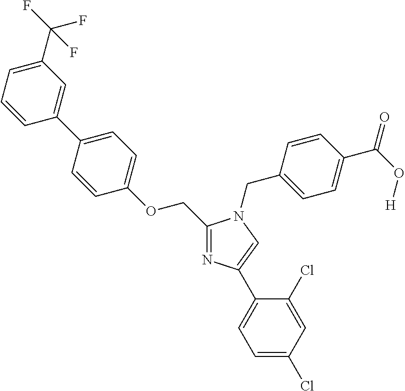 | 4-[4-(2,4-Dichloro- phenyl)-2-(3′- trifluoromethyl-biphenyl- 4-yloxymethyl)-imidazol- 1-ylmethyl]-benzoic acid |
| 287 | 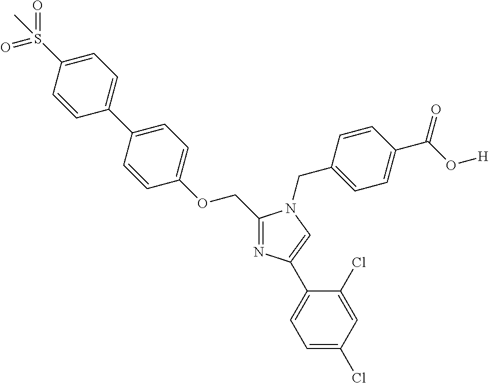 | 4-[4-(2,4-Dichloro- phenyl)-2-(4′- methanesulfonyl- biphenyl-4-yloxymethyl)- imidazol-1-ylmethyl]- benzoic acid |
| 288 | 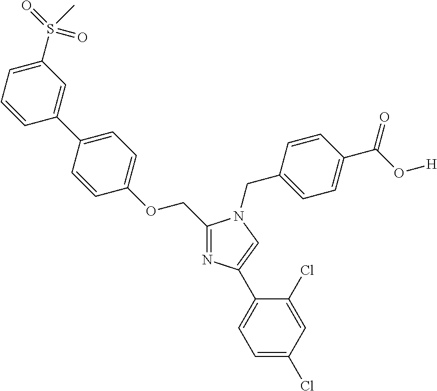 | 4-[4-(2,4-Dichloro- phenyl)-2-(3′- methanesulfonyl- biphenyl-4-yloxymethyl)- imidazol-1-ylmethyl]- benzoic acid |
| 289 | 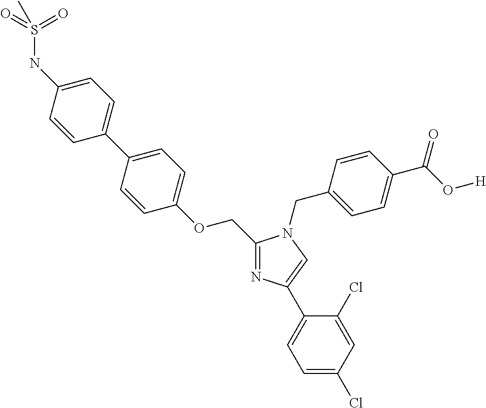 | 4-[4-(2,4-Dichloro- phenyl)-2-(4′- methanesulfonylamino- biphenyl-4-yloxymethyl)- imidazol-1-ylmethyl]- benzoic acid |
| 290 | 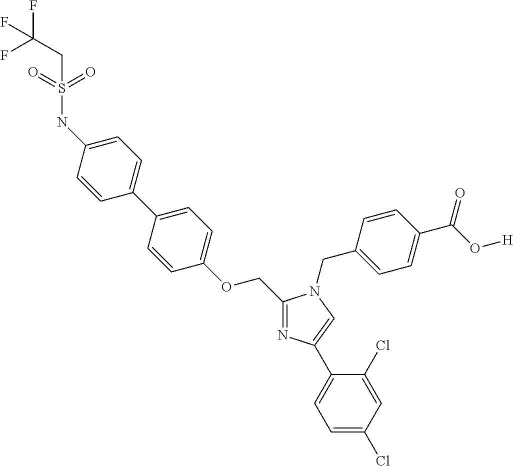 | 4-{4-(2,4-Dichloro- phenyl)-2-[4′-(2,2,2- trifluoro- ethanesulfonylamino)- biphenyl-4-yloxymethyl]- imidazol-1-ylmethyl}- benzoic acid |
| 291 | 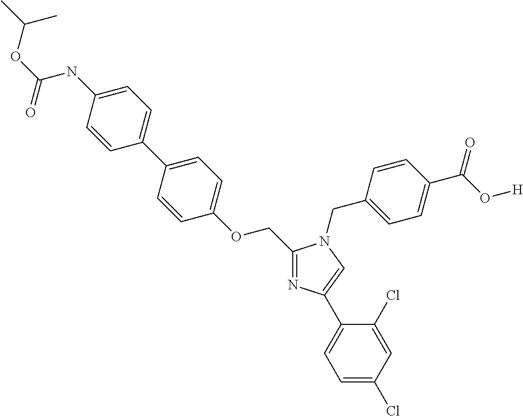 | 4-[4-(2,4-Dichloro- phenyl)-2-(4′- isopropoxycarbonylamino- biphenyl-4- yloxymethyl)-imidazol-1- ylmethyl]-benzoic acid |
| 292 | 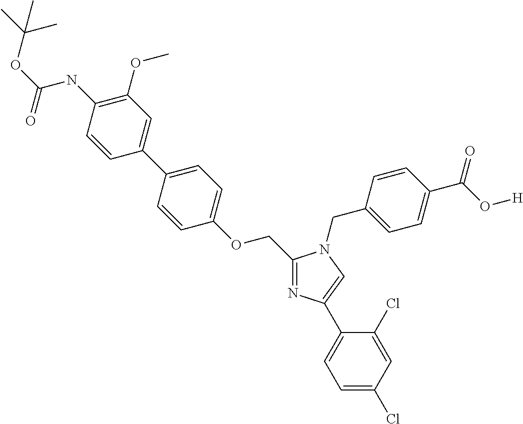 | 4-[2-(4′-tert- Butoxycarbonylamino-3′- methoxy-biphenyl-4- yloxymethyl)-4-(2,4- dichloro-phenyl)- imidazol-1-ylmethyl]- benzoic acid |
| 293 | 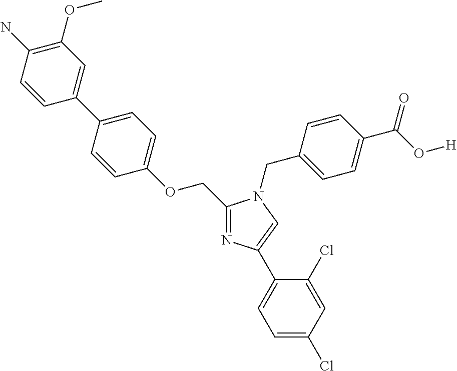 | 4-[2-(4′-Amino-3′- methoxy-biphenyl-4- yloxymethyl)-4-(2,4- dichloro-phenyl)- imidazol-1-ylmethyl]- benzoic acid |
| 294 | 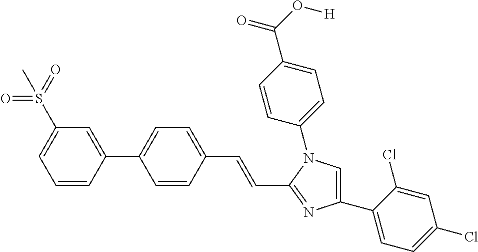 | 4-{4-(2,4-Dichloro- phenyl)-2-[2-(3′- methanesulfonyl- biphenyl-4-yl)-(E)-vinyl]- imidazol-1-yl}-benzoic acid |
| 295 | 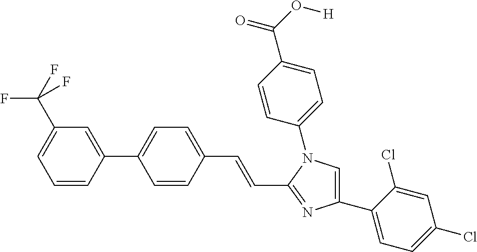 | 4-{4-(2,4-Dichloro- phenyl)-2-[2-(3′- trifluoromethyl-biphenyl- 4-yl)-(E)-vinyl]-imidazol- 1-yl}-benzoic acid |
| 296 | 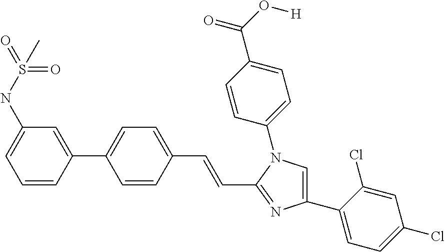 | 4-{4-(2,4-Dichloro- phenyl)-2-[2-(3′- methanesulfonylamino- biphenyl-4-yl)-(E)-vinyl]- imidazol-1-yl}-benzoic acid |
| 297 | 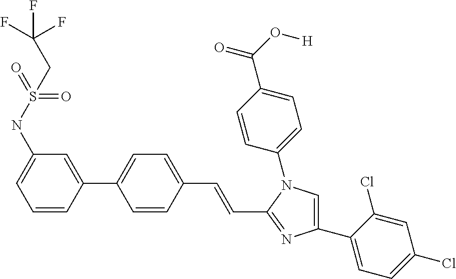 | 4-(4-(2,4-Dichloro- phenyl)-2-{2-[3′-(2,2,2- trifluoro- ethanesulfonylamino)- biphenyl-4-yl]-(E)-vinyl}- imidazol-1-yl)-benzoic acid |
| 298 | 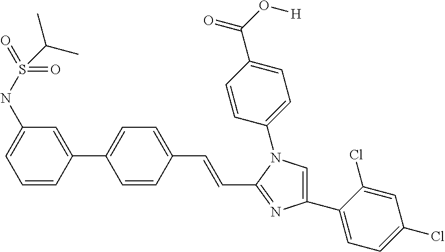 | 4-(4-(2,4-Dichloro- phenyl)-2-{2-[3′- (propane-2- sulfonylamino)-biphenyl- 4-yl]-(E)-vinyl}-imidazol- 1-yl)-benzoic acid |
| 299 | 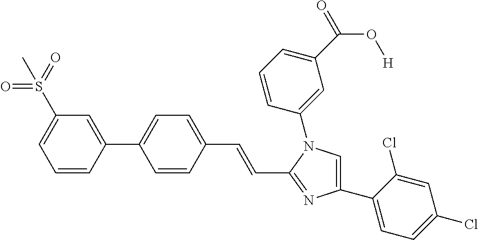 | 3-{4-(2,4-Dichloro- phenyl)-2-[2-(3′- methanesulfonyl- biphenyl-4-yl)-(E)-vinyl]- imidazol-1-yl}-benzoic acid |
| 300 | 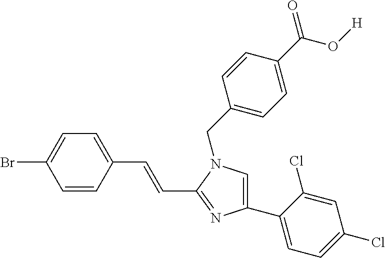 | 4-[2-[2-(4-Bromo- phenyl)-(E)-vinyl]-4-(2,4- dichloro-phenyl)- imidazol-1-ylmethyl]- benzoic acid |
| 301 | 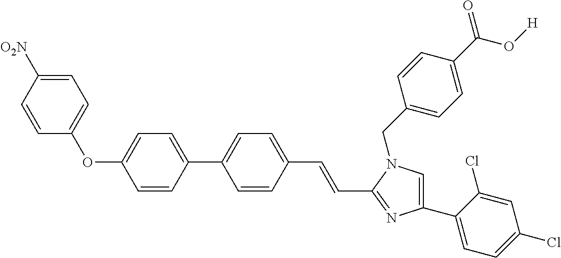 | 4-[2-{2-[4′-(4-Nitro- phenoxy)-biphenyl-4-yl]- (E)-vinyl}-4-(2,4-dichloro- phenyl)-imidazol-1- ylmethyl]-benzoic acid |
| 302 | 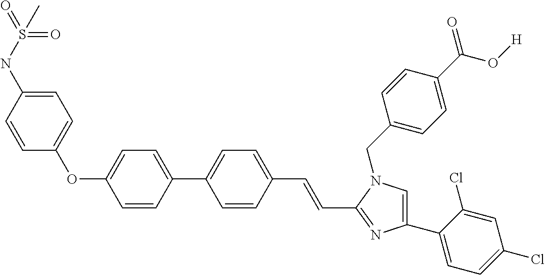 | 4-(4-(2-4-Dichloro- phenyl)-2-{2-[4′-(4- methanesulfonylamino- phenoxy)-biphenyl-4yl]- (E)-vinyl}-imidazol-1-yl- methyl)-benzoic acid |
| 303 | 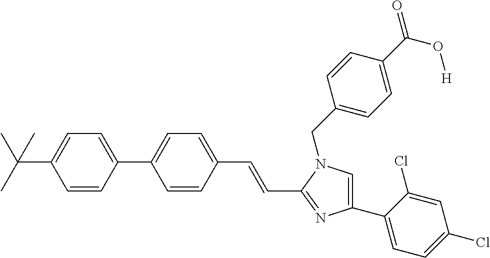 | 4-[2-[2-(4′-tert-Butyl- biphenyl-4-yl)-(E)-vinyl]- 4-(2,4-dichloro-phenyl)- imidazol-1-ylmethyl]- benzoic acid |
| 304 | 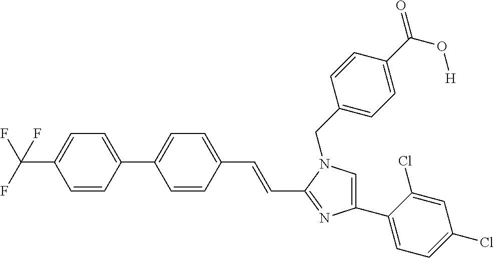 | 4-{4-(2,4-Dichloro- phenyl)-2-[2-(4′- trifluoromethyl-biphenyl- 4-yl)-(E)-vinyl]-imidazol- 1-ylmethyl}-benzoic acid |
| 305 | 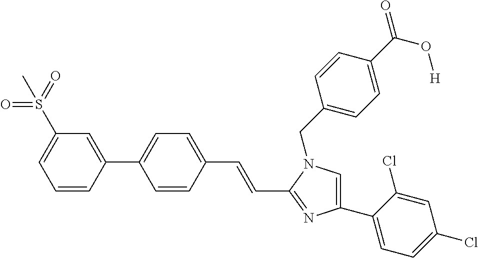 | 4-{4-(2,4-Dichloro- phenyl)-2-[2-(3′- methanesulfonyl- biphenyl-4-yl)-(E)-vinyl]- imidazol-1-ylmethyl}- benzoic acid |
| 306 | 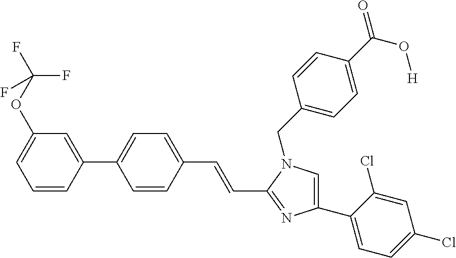 | 4-{4-(2,4-Dichloro- phenyl)-2-[2-(3′- trifluoromethoxy- biphenyl-4-yl)-(E)-vinyl]- imidazol-1-ylmethyl}- benzoic acid |
| 307 | 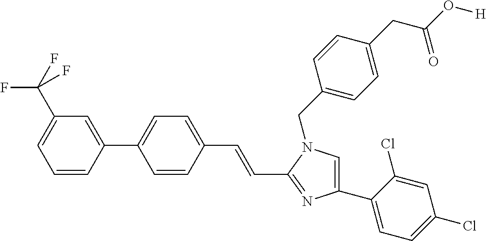 | (4-{4-(2,4-Dichloro- phenyl)-2-[2-(3′- trifluoromethyl-biphenyl- 4-yl)-(E)-vinyl]-imidazol- 1-ylmethyl}-phenyl) acetic acid |
| 308 | 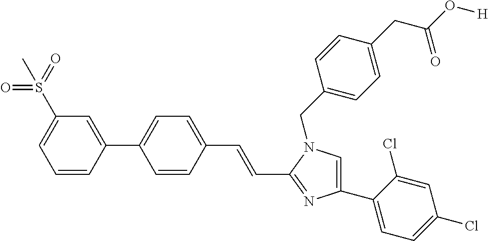 | (4-{4-(2,4-Dichloro- phenyl)-2-[2-(3′- methanesulfonyl- biphenyl-4-yl)-(E)-vinyl]- imidazol-1-ylmethyl}- phenyl)-acetic acid |
| 309 | 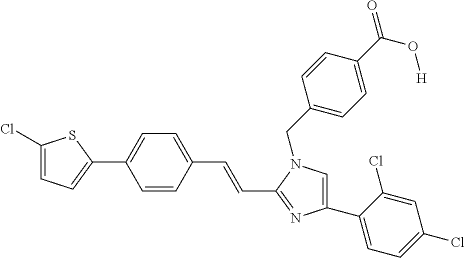 | 4-[2-{2-[4-(5-Chloro- thiophen-2-yl)-phenyl]- (E)-vinyl}-4-(2,4-dichloro- phenyl)-imidazol-1- ylmethyl]-benzoic acid |
| 310 | 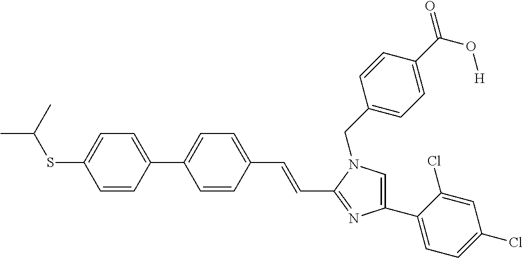 | 4-{4-(2,4-Dichloro- phenyl)-2-[2-(4′- isopropylsulfanyl- biphenyl-4-yl)-(E)-vinyl]- imidazol-1-ylmethyl}- benzoic acid |
| 311 | 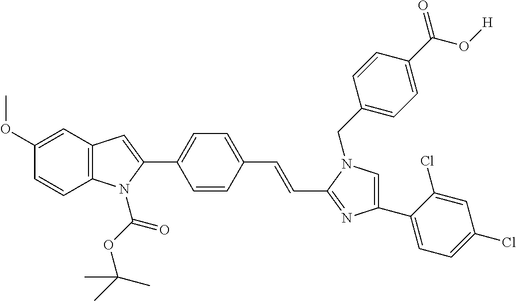 | 2-(4-{2-[1-(4-Carboxy- benzyl)-4-(2,4-dichloro- phenyl)-1H-imidazol-2- yl]-(E)-vinyl}-phenyl)-5- methoxy-indole-1- carboxylic acid tert-butyl ester |
| 312 | 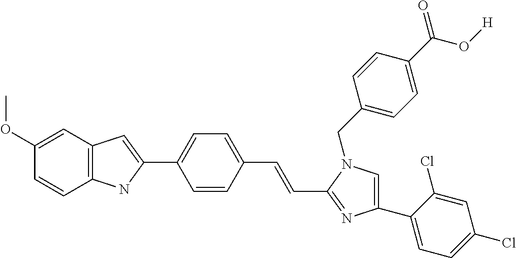 | 4-(4-(2,4-Dichloro- phenyl)-2-{2-[4-(5- methoxy-1H-indol-2-yl)- phenyl]-(E)-vinyl}- imidazol-1-ylmethyl)- benzoic acid |
| 313 | 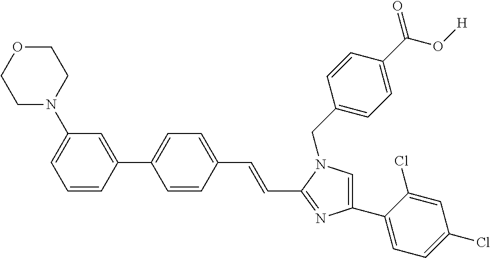 | 4-{4-(2,4-Dichloro- phenyl)-2-[2-(3′- morpholin-4-yl-biphenyl- 4-yl)-(E)-vinyl]-imidazol- 1-ylmethyl}-benzoic acid |
| 314 | 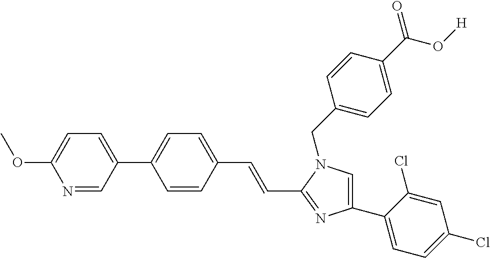 | 4-{4-(2,4-Dichloro- phenyl)-2-[2-[4-(6- methoxy-pyridin-3-yl)- phenyl]-(E)-vinyl}- imidazol-1-ylmethyl)- benzoic acid |
| 315 | 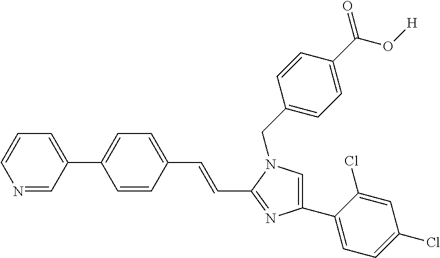 | 4-{4-(2,4-Dichloro- phenyl)-2-[2-[4-pyridin-3- yl)-phenyl]-(E)-vinyl}- imidazol-1-ylmethyl)- benzoic acid |
| 316 | 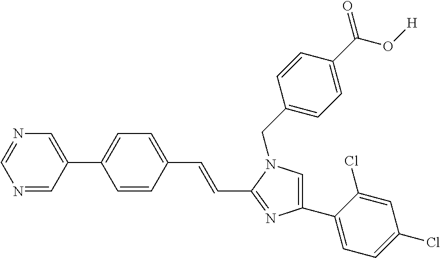 | 4-{4-(2,4-Dichloro- phenyl)-2-[2-[4-pyrimidin- 3-yl)-phenyl]-(E)-vinyl}- imidazol-1-ylmethyl)- benzoic acid |
| 317 | 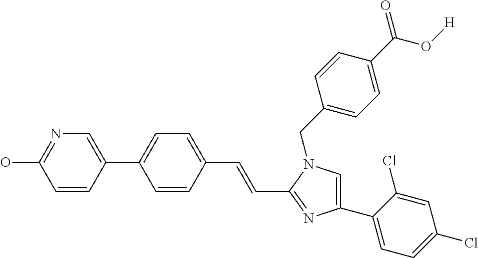 | 4-{4-(2,4-Dichloro- phenyl)-2-[2-[4-(6- hydroxy-pyridin-3-yl)- phenyl]-(E)-vinyl}- imidazol-1-ylmethyl)- benzoic acid |
| 318 | 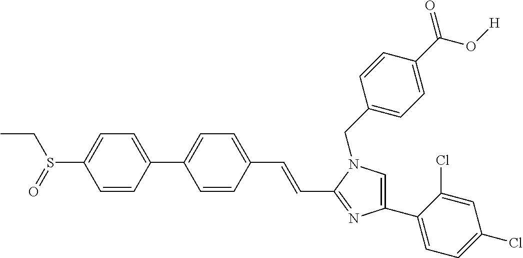 | 4-{4-(2,4-Dichloro- phenyl)-2-[2-(4′- ethanesulfinyl-biphenyl- 4-yl)-(E)-vinyl]-imidazol- 1-ylmethyl}-benzoic acid |
| 319 | 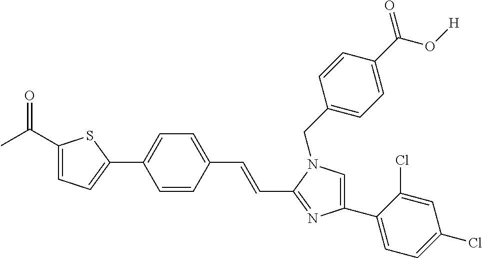 | 4-[2-{2-[4-(5-Acetyl- thiophen-2-yl-phenyl]- (E)-vinyl}-4-(2,4-dichloro- phenyl)-imidazol-1- ylmethyl]-benzoic acid |
| 320 | 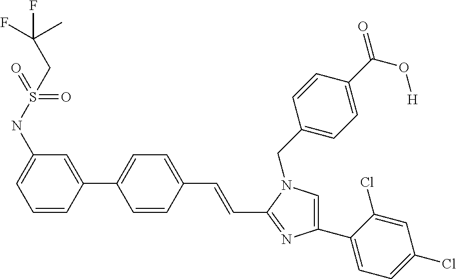 | 4-(4-(2,4-Dichloro- phenyl)-2-{2-[3′-(2,2,2- trifluoro- ethanesulfonylamino)- biphenyl-4-yl]-(E)-vinyl}- imidazol-1-ylmethyl)- benzoic acid |
| 321 | 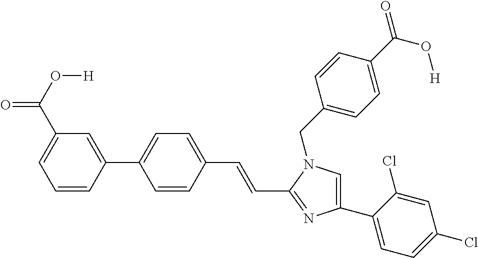 | 4′-{2-[1-(4-Carboxy- benzyl)-4-(2,4-dichloro- phenyl)-1H-imidazol-2- yl]-(E)-vinyl}-biphenyl-3- carboxylic acid |
| 322 | 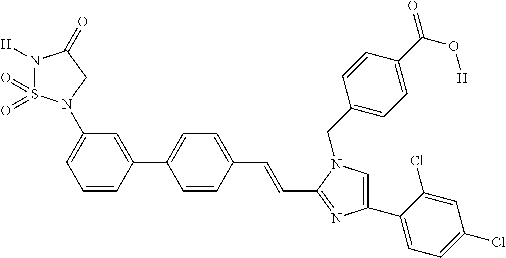 | 4-(4-(2,4-Dichloro- phenyl)-2-{2-[3′-(1,1,4- trioxo-1-[1,2,5]- thiadiazolidin-2-yl)- biphenyl-4-yl]-(E)-vinyl}- imidazol-1-ylmethyl)- benzoic acid |
| 323 | 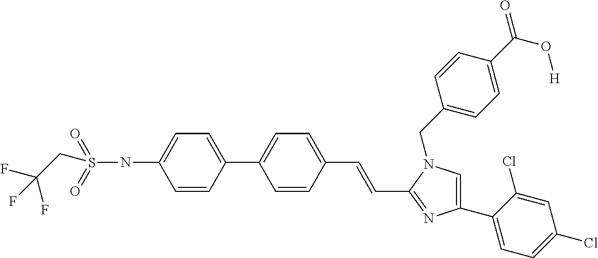 | 4-(4-(2,4-Dichloro- phenyl)-2-{2-[4′-(2,2,2- trifluoro- ethanesulfonylamino)- biphenyl-4-yl]-(E)-vinyl}- imidazol-1-ylmethyl)- benzoic acid |
| 324 | 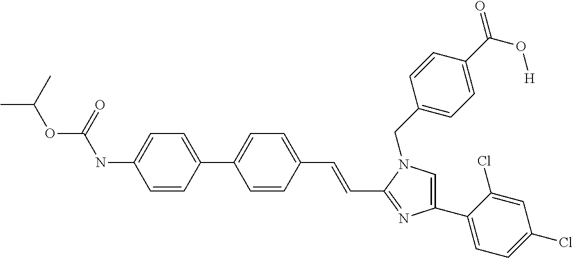 | 4-{4-(2,4-Dichloro- phenyl)-2-[2-(4′- isopropoxycarbonylamino- biphenyl-4-yl)-(E)- vinyl]-imidazol-1- ylmethyl}-benzoic acid |
| 325 | 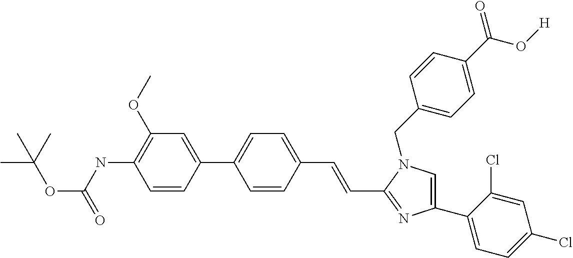 | 4-[2-[2-(4′-tert- Butoxycarbonylamino-3′- methoxy-biphenyl-4-yl)- (E)-vinyl]-4-(2,4-dichloro- phenyl)-imidazol-1- ylmethyl]-benzoic acid |
| 326 | 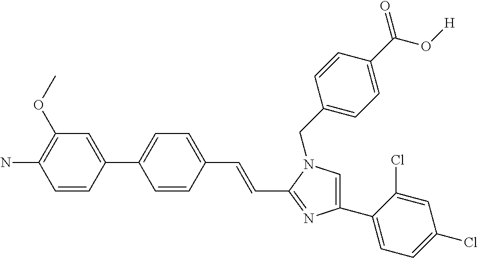 | 4-[2-[2-(4′-Amino-3′- methoxy-biphenyl-4-yl)- (E)-vinyl]-4-(2,4-dichloro- phenyl)-imidazol-1- ylmethyl]-benzoic acid |
| 327 | 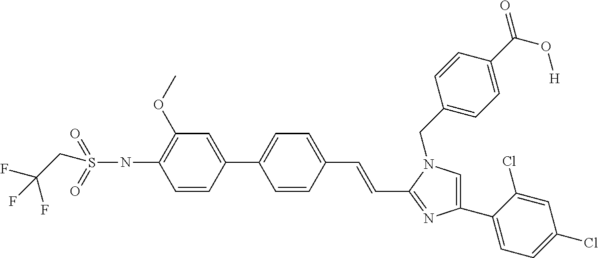 | 4-(4-(2,4-Dichloro- phenyl)-2-{2-[3′-methoxy- 4′-(2,2,2-trifluoro- ethanesulfonylamino)- biphenyl-4-yl]-(E)-vinyl}- imidazol-1-ylmethyl)- benzoic acid |
| 328 | 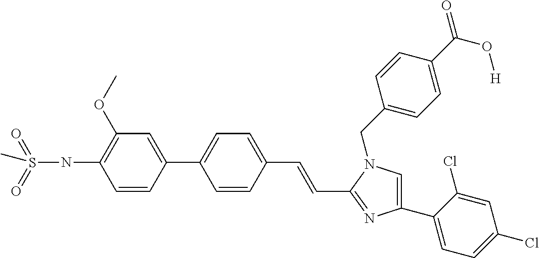 | 4-{4-(2,4-Dichloro- phenyl)-2-[2-(4′- methanesulfonylamino- 3′-methoxy-biphenyl-4- yl)-(E)-vinyl]-imidazol-1- ylmethyl}-benzoic acid |
| 329 | 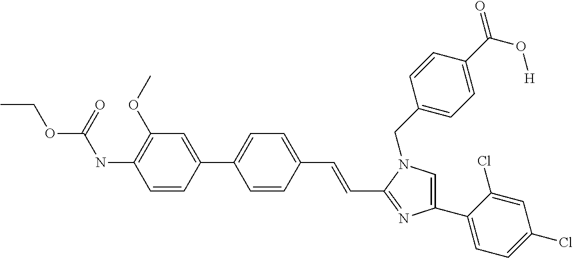 | 4-{4-(2,4-Dichloro- phenyl)-2-[2-(4′- ethoxycarbonylamino-3′- methoxy-biphenyl-4-yl)- (E)-vinyl]-imidazol-1- ylmethyl}-benzoic acid |
| 330 | 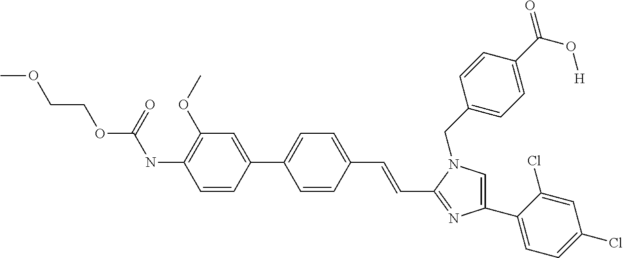 | 4-(4-(2,4-Dichloro- phenyl)-2-{2-[3′-methoxy- 4′-(2-methoxy- ethoxycarbonylamino)- biphenyl-4-yl]-(E)-vinyl}- imidazol-1-ylmethyl)- benzoic acid |
| 331 | 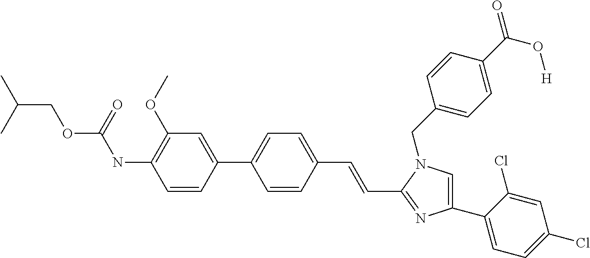 | 4-{4-(2,4-Dichloro- phenyl)-2-[2-(4′- isobutoxycarbonylamino- 3′-methoxy-biphenyl-4- yl)-(E)-vinyl]-imidazol-1- ylmethyl}-benzoic acid |
| 332 | 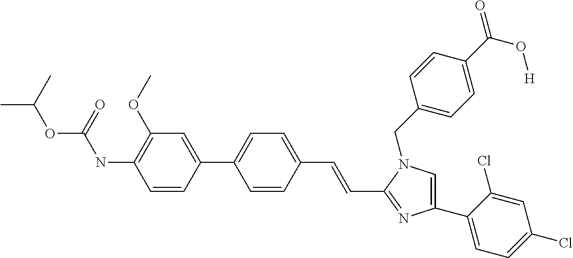 | 4-{4-(2,4-Dichloro- phenyl)-2-[2-(4′- isopropoxycarbonylamino- 3′-methoxy-biphenyl-4- yl)-(E)-vinyl]-imidazol-1- ylmethyl}-benzoic acid |
| 333 | 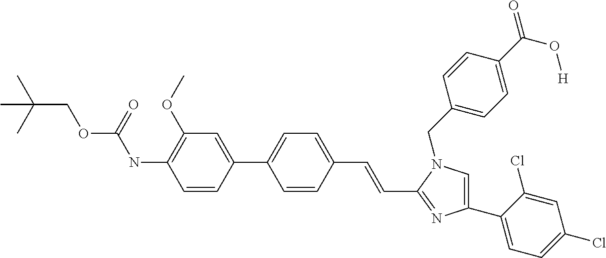 | 4-(4-(2,4-Dichloro- phenyl)-2-{2-[4′-(2,2- dimethyl- propoxycarbonylamino)- 3′-methoxy biphenyl-4-yl]-(E)-vinyl}- imidazol-1-ylmethyl)- benzoic acid |
| 334 | 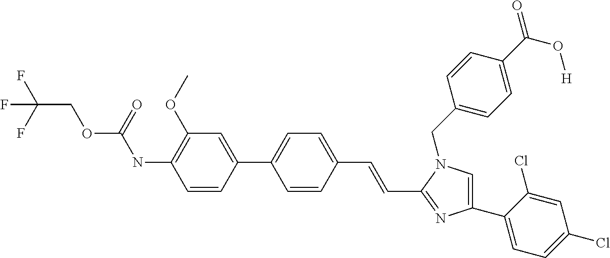 | 4-(4-(2,4-Dichloro- phenyl)-2-{2-[3′-methoxy- 4′-(2,2,2-trifluoro- ethoxycarbonylamino)- biphenyl-4-yl]-(E)-vinyl}- imidazol-1-ylmethyl)- benzoic acid |
| 335 | 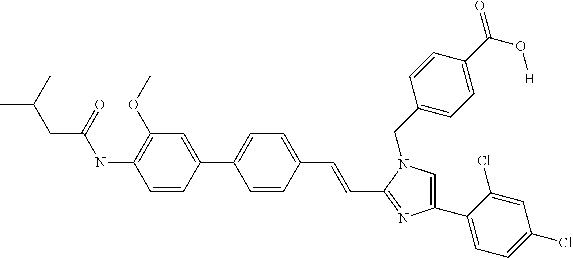 | 4-(4-(2,4-Dichloro- phenyl)-2-{2-[3′-methoxy- 4′-(3-methyl- butyrylamino)-biphenyl- 4-yl]-(E)-vinyl}-imidazol- 1-ylmethyl)-benzoic acid |
| 336 | 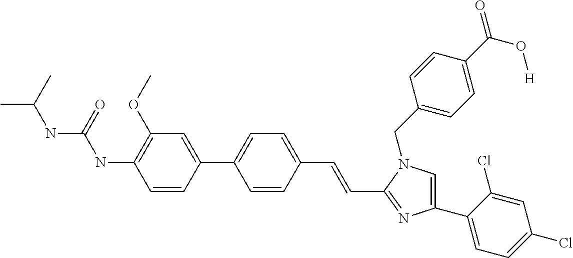 | 4-(4-(2,4-Dichloro- phenyl)-2-{2-[4′-(3- isopropyl-ureido)-3′- methoxy-biphenyl-4-yl]- (E)-vinyl}-imidazol-1- ylmethyl)-benzoic acid |
| 337 | 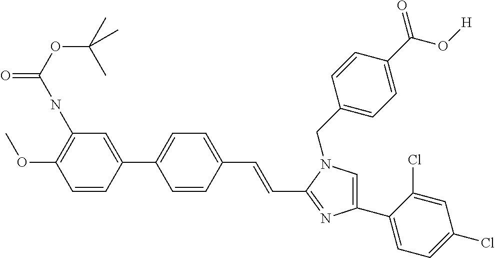 | 4-[2-[2-(3′-tert- Butoxycarbonylamino-4′- methoxy-biphenyl-4-yl)- (E)-vinyl]-4-(2,4-dichloro- phenyl)-imidazol-1- ylmethyl]-benzoic acid |
| 338 | 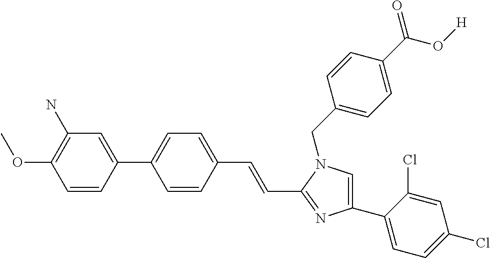 | 4-[2-[2-(3′-Amino-4′- methoxy-biphenyl-4-yl)- (E)-vinyl]-4-(2,4-dichloro- phenyl)-imidazol-1- ylmethyl]-benzoic acid |
| 339 | 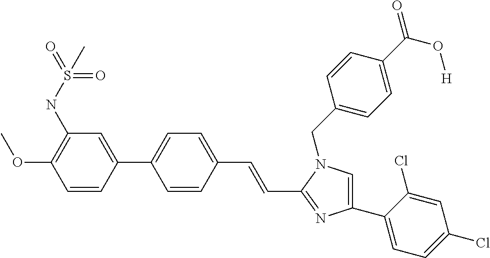 | 4-{4-(2,4-Dichloro- phenyl)-2-[2-(3′- methanesulfonylamino- 4′-methoxy-biphenyl-4- yl)-(E)-vinyl]-imidazol-1- ylmethyl}-benzoic acid |
| 340 | 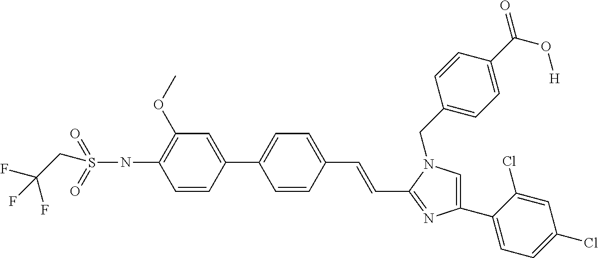 | 4-(4-(2,4-Dichloro- phenyl)-2-{2-[4′-methoxy- 3′-(2,2,2-trifluoro- ethanesulfonylamino)- biphenyl-4-yl]-(E)-vinyl}- imidazol-1-ylmethyl)- benzoic acid |
| 341 | 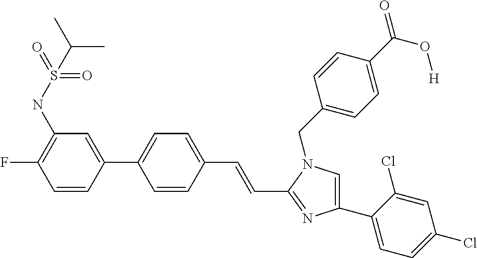 | 4-(4-(2,4-Dichloro- phenyl)-2-{2-[4′-fluoro-3′- (propane-2- sulfonylamino)-biphenyl- 4-yl]-(E)-vinyl}-imidazol- 1-ylmethyl)-benzoic acid |
| 342 | 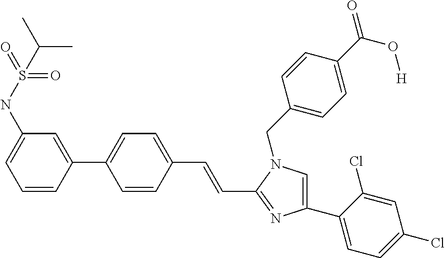 | 4-(4-(2,4-Dichloro- phenyl)-2-{2-[3′- (propane-2- sulfonylamino)-biphenyl- 4-yl]-(E)-vinyl}-imidazol- 1-ylmethyl)-benzoic acid |
| 343 | 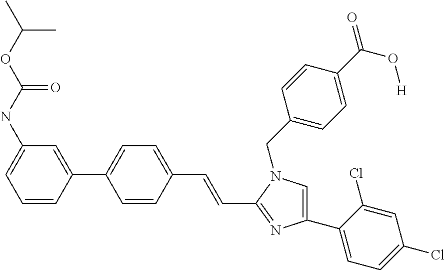 | 4-{4-(2,4-Dichloro- phenyl)-2-[2-(3′- isopropoxycarbonylamino- biphenyl-4-yl)-(E)- vinyl]-imidazol-1- ylmethyl}-benzoic acid |
| 344 | 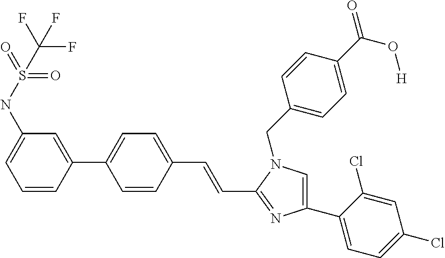 | 4-{4-(2,4-Dichloro- phenyl)-2-[2-(3′- trifluoromethanesulfonyl- amino-biphenyl-4-yl)-(E)- vinyl]imidazol-1- ylmethyl}-benzoic acid |
| 345 | 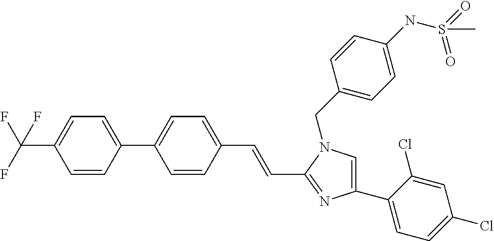 | N-(4-{4-(2,4-Dichloro- phenyl)-2-[2-(4′- trifluoromethyl-biphenyl- 4-yl)-(E)-vinyl]-imidazol- 1ylmethyl}-phenyl)- methanesulfonamide |
| 346 | 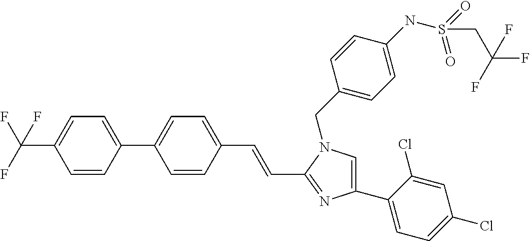 | 2,2,2-Trifluoro- ethanesulfonic acid (4- {4-(2,4-dichloro-phenyl)- 2-[2-(4′-trifluoromethyl biphenyl-4-yl)-(E)-vinyl]- imidazol-1-ylmethyl}- phenyl)-amide |
| 347 | 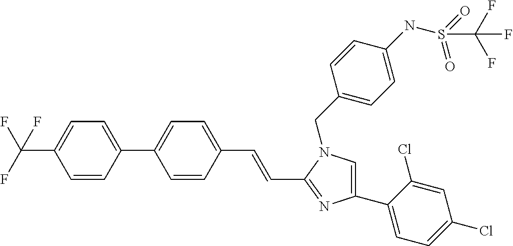 | N-(4-{4-(2,4-Dichloro- phenyl)-2-[2-(4′-trifluoro- methyl-biphenyl-4-yl)- (E)-vinyl]-imidazol-1- ylmethyl}-phenyl)- trifluoro- methanesulfonamide |
| 348 | 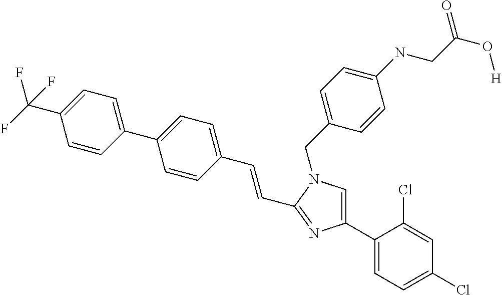 | (4-{4-(2,4-Dichloro- phenyl)-2-[2-(4′- trifluoromethyl-biphenyl- 4-yl)-(E)-vinyl]-imidazol- 1-ylmethyl}- phenylamino)-acetic acid |
| 349 | 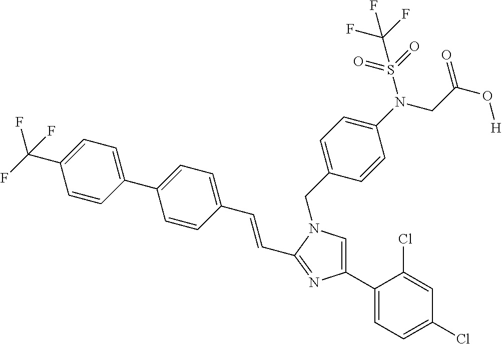 | [(4-{4-(2,4-Dichloro- phenyl)-2-[2-(4′- trifluoromethyl-biphenyl- 4-yl)-(E)-vinyl]-imidazol- 1-ylmethyl}-phenyl)- trifluoromethanesulfonyl- amino]-acetic acid |
| 350 | 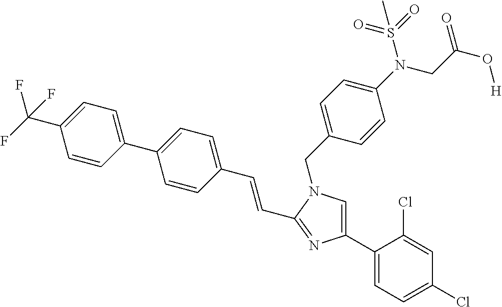 | [(4-{4-(2,4-Dichloro- phenyl)-2-[2-(4′- trifluoromethyl-biphenyl- 4-yl)-(E)-vinyl]-imidazol- 1-ylmethyl}-phenyl)- methanesulfonyl-amino]- acetic acid |
| 351 | 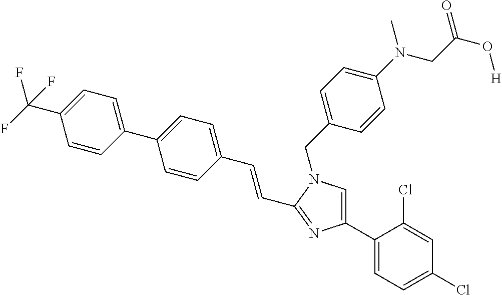 | [(4-{4-(2,4-Dichloro- phenyl)-2-[2-(4′- trifluoromethyl-biphenyl- 4-yl)-(E)-vinyl]-imidazol- 1-ylmethyl}-phenyl)- methyl-amino]-acetic acid |
| 352 | 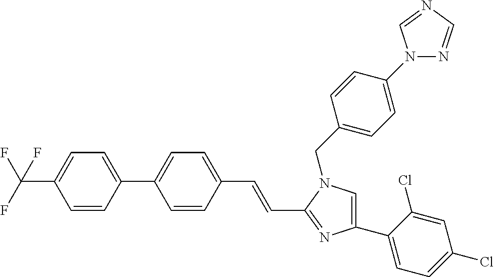 | 1-(4-{4-(2,4-Dichloro- phenyl)-2-[2-(4′- trifluoromethyl-biphenyl- 4-yl)-(E)-vinyl]-imidazol-1 ylmethyl}-phenyl)-1H- [1,2,4]-triazole |
| 353 | 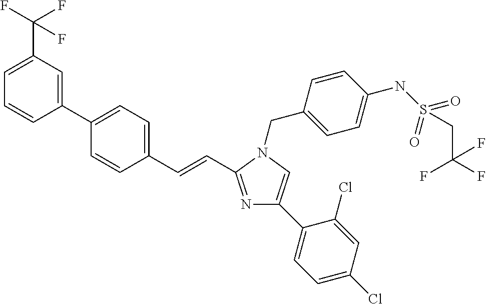 | 2,2,2-Trifluoro- ethanesulfonic acid (4- {4-(2,4-dichloro-phenyl)- 2-[2-(3′-trifluoromethyl- biphenyl-4-yl)-(E)-vinyl]- imidazol-1-ylmethyl}- phenyl)-amide |
| 354 | 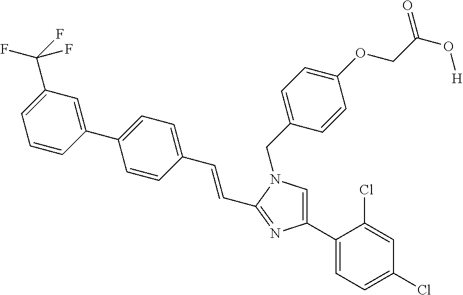 | (4-{4-(2,4-Dichloro- phenyl)-2-[2-(3′- trifluoromethyl-biphenyl- 4-yl)-(E)-vinyl]-imidazol- 1-ylmethyl}-phenoxy)- acetic acid |
| 355 | 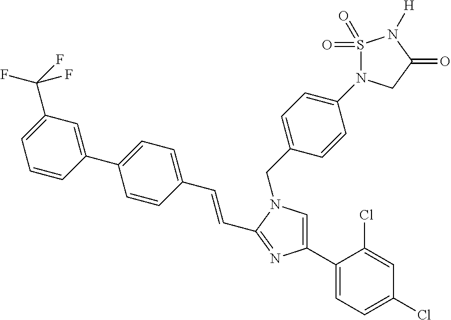 | 5-(4-{4-(2,4-Dichloro- phenyl)-2-[2-(3′- trifluoromethyl-biphenyl- 4-yl)-(E)-vinyl]-imidazol-1 ylmethyl}-phenyl)-1,2,5- thiadiazolidin-3-one-1,1- dioxide |
| 356 | 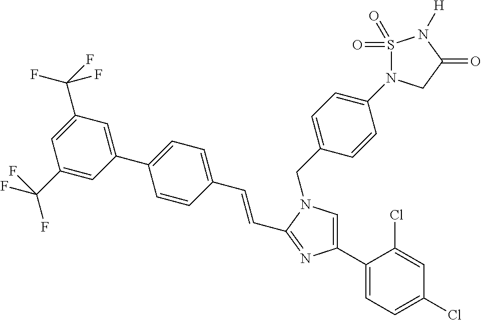 | 5-{4-[2-[2-(3′,5′ Bistrifluoromethyl- biphenyl- 4-yl)-(E)-vinyl]-4-(2,4- dichlorophenyl) imidazol-1-ylmethyl]- phenyl}-1,2,5- thiadiazolidin-3-one-1,1- dioxide |
| 357 | 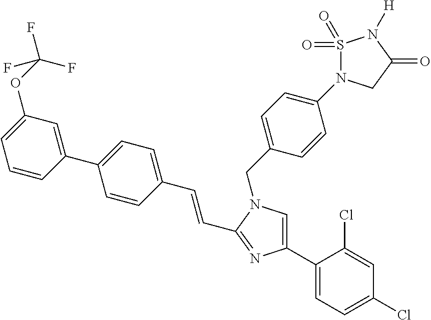 | 5-(4-{4-(2,4-Dichloro- phenyl)-2 [2-(3′-trifluoromethoxy- biphenyl 4-yl)-(E)-vinyl]-imidazol-1 ylmethyl}-phenyl)-1,2,5- thiadiazolidin-3-one-1,1- dioxide |
| 358 | 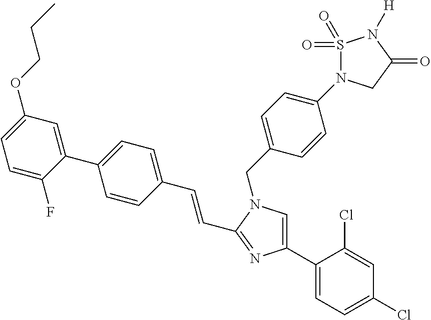 | 5-(4-{4-(2,4-Dichloro- phenyl)-2 [2-(2′-fluoro-5′-propoxy- biphenyl 4-yl)-(E)-vinyl]-imidazol-1 ylmethyl}-phenyl)-1,2,5- thiadiazolidin-3-one-1,1- dioxide |
| 359 | 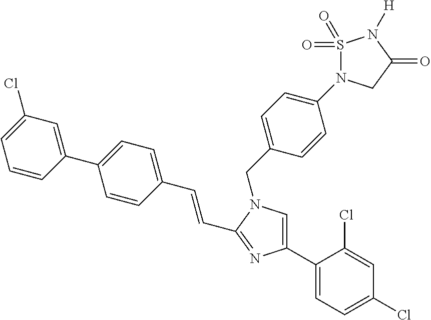 | 5-{4-[2-[2-(3′-Chloro- biphenyl-4 yl)-(E)-vinyl]-4-(2,4- dichloro-phenyl) imidazol-1ylmethyl] phenyl}-1,2,5- thiadiazolidin-3-one-1,1- dioxide |
| 360 | 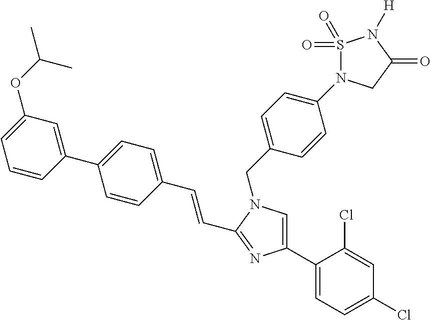 | 5-(4-{4-(2,4-Dichloro- phenyl)-2 [2-(3′-isopropoxy- biphenyl-4-yl) vinyl]-imidazol- ylmethyl}-phenyl)-1,2,5- thiadiazolidin-3-one-1,1- dioxide |
| 361 | 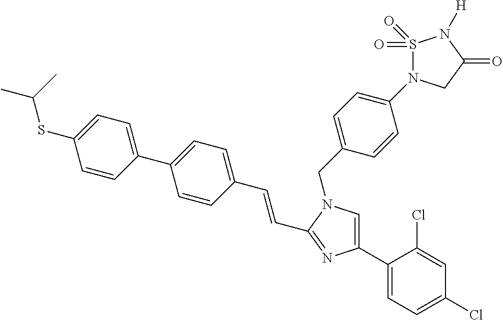 | 5-(4-{4-(2,4-Dichloro- phenyl)-2 [2-(4′-isopropylsulfanyl- biphenyl 4-yl)-(E)-vinyl]-imidazol-1 ylmethyl}-phenyl)-1,2,5- thiadiazolidin-3-one-1,1- dioxide |
| 362 | 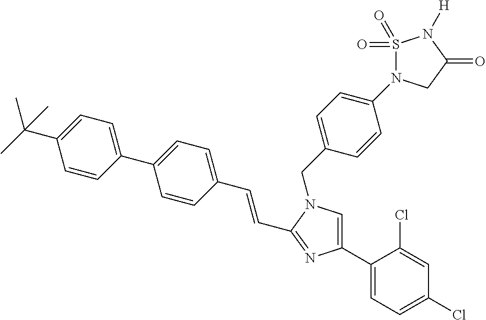 | 5-{4-[2-[2-(4′-tert- Butylbiphenyl4-yl)-(E)- vinyl]-4-(2,4- dichlorophenyl)-imidazol- 1-ylmethyl]phenyl}-1,2,5- thiadiazolidin-3-one-1,1- dioxide |
| 363 | 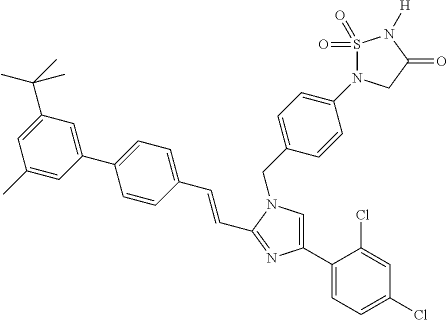 | 5-{4-[2-[2-(3′-tert-Butyl- 5′-methyl biphenyl-4-yl)-(E)-vinyl]- 4-(2,4 dichloro-phenyl)- imidazol-1 ylmethyl]-phenyl}-1,2,5- thiadiazolidin-3-one-1,1- dioxide |
| 364 | 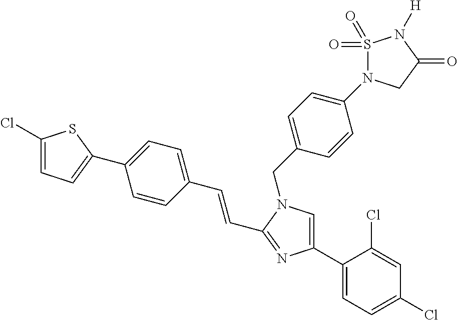 | 5-{4-[2-{2-[4-(5-Chloro- thiophen 2-yl)-phenyl]-(E)-vinyl}-4- (2,4 dichloro-phenyl)- imidazol-1 ylmethyl]-phenyl}-1,2,5- thiadiazolidin-3-one-1,1- dioxide |
| 365 | 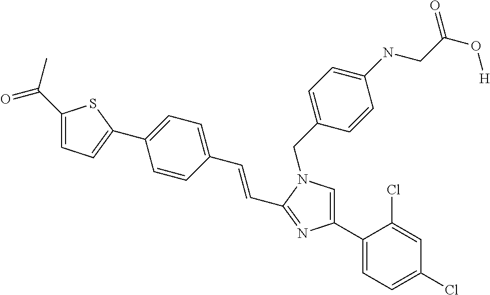 | {4-[2-{2-[4-(5-Acetyl- thiophen-2 yl)-phenyl]-(E)-vinyl}-4- (2,4-dichloro phenyl)-imidazol-1- ylmethyl]- phenylamino}-acetic acid |
| 366 | 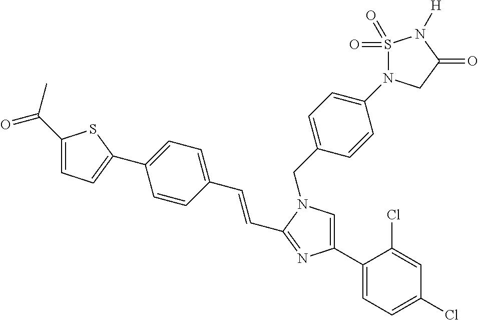 | 5-{4-[2-{2-[4-(5-Acetyl- thiophen 2-yl)-phenyl]-(E)-vinyl}-4- (2,4 dichloro-phenyl)- imidazol-1 ylmethyl]-phenyl}-1,2,5- thiadiazolidin-3-one-1,1- dioxide |
| 367 | 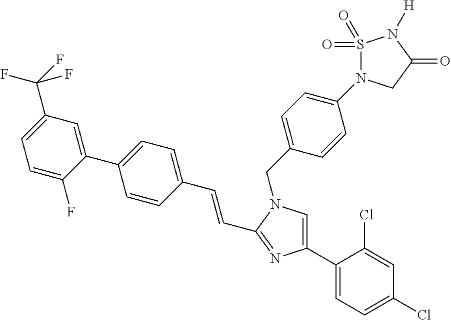 | 5-(4-{4-(2,4-Dichloro- phenyl)-2 [2-(2′-fluoro-5′- trifluoromethyl biphenyl-4-yl)-(E)-vinyl] imidazol-1-ylmethyl}- phenyl)-1,2,5- thiadiazolidin-3-one-1,1- dioxide |
| 368 | 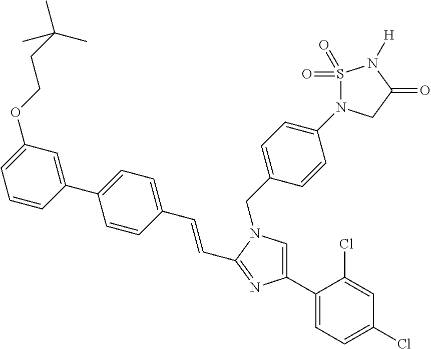 | 5-[4-(4-(2,4-Dichloro- phenyl)-2 {2-[3′-(3,3-dimethyl- butoxy) biphenyl-4-yl]-(E)-vinyl}- imidazol 1-ylmethyl)-phenyl]- 1,2,5-thiadiazolidin-3- one-1,1-dioxide |
| 369 | 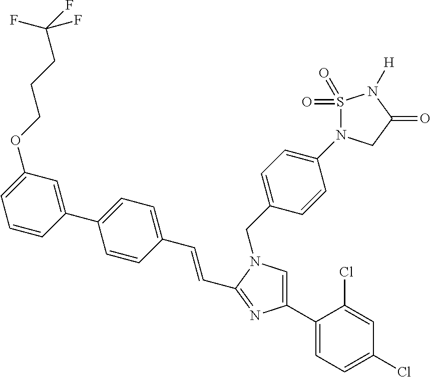 | 5-[4-(4-(2,4-Dichloro- phenyl)-2 {2-[3′-(4,4,4-trifluoro- butoxy) biphenyl-4-yl]-(E)-vinyl}- imidazol-1-ylmethyl)- phenyl]-1,2,5- thiadiazolidin-3-one-1,1- dioxide |
| 370 | 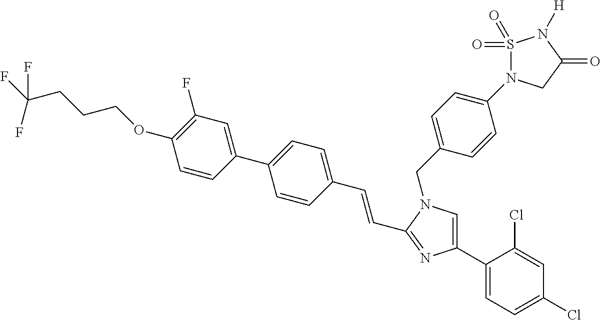 | 5-[4-(4-(2,4-Dichloro- phenyl)-2 {2-[3′-fluoro-4′-(4,4,4- trifluoro butoxy)-biphenyl-4-yl]- vinyl}-imidazol-1- ylmethyl)-phenyl]-1,2,5- thiadiazolidin-3-one-1,1- dioxide |
| 371 | 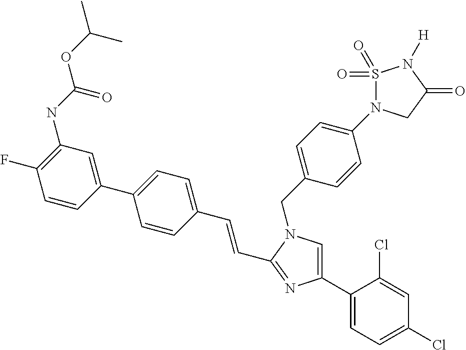 | [4′-(2-{4-(2,4-Dichloro- phenyl)-1-[4-(1,1,4- trioxo-1- [1,2,5]thiadiazolidin-2- yl)benzyl]-1H-imidazol-2- yl}-(E)-vinyl)-4-fluoro- biphenyl-3-yl]-carbamic acid isopropyl ester |
| 372 | 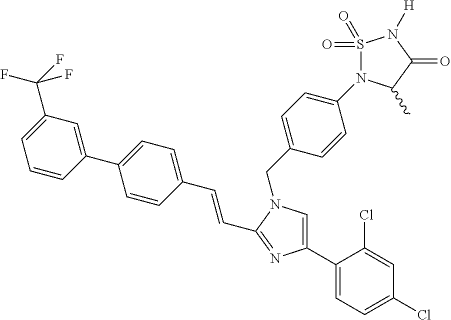 | 5-(4-{4-(2,4-Dichloro- phenyl)-2 [2-(3′-trifluoromethyl- biphenyl-4 yl)-(E)-vinyl]-imidazol-1 ylmethyl}-phenyl)-4- methyl-,2,5- thiadiazolidin-3-one-1,1- dioxide |
| 373 | 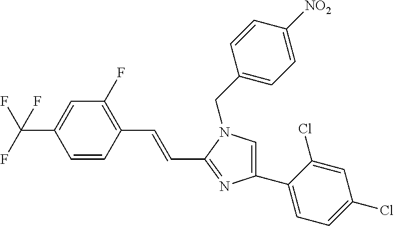 | 4-(2,4-Dichloro-phenyl)- 2-[2-(2-fluoro-4- trifluoromethyl-phenyl)- (E)-vinyl]-1-(4-nitro- benzyl)-1H- imidazole |
| 374 | 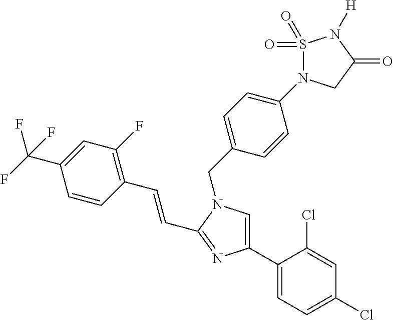 | 5-(4-{4-(2,4-Dichloro- phenyl)-2-[2-(2-fluoro-4- trifluoromethyl-phenyl)- (E)-vinyl]-imidazol-1- ylmethyl}-phenyl)-1,2,5- thiadiazolidin-3-one-1,1- dioxide |
| 375 | 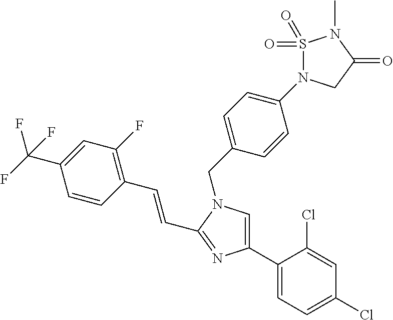 | 5-(4-{4-(2,4-Dichloro- phenyl)-2-[2-(2-fluoro-4- trifluoromethyl-phenyl)- (E)-vinyl]-imidazol-1 ylmethyl}-phenyl)-2- methyl-2,5-thiadiazolidin- 3-on-1,1-dioxide |
| 376 | 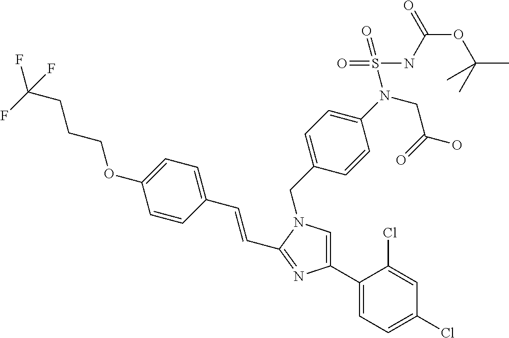 | {[4-(4-(2,4-Dichloro- phenyl)-2-{2 [4-(4,4,4-trifluoro-butoxy) phenyl]-(E)-vinyl}- imidazol-1- ylmethyl)-phenyl]-N-Boc- sulfonyl-amino}-acetic acid |
| 377 | 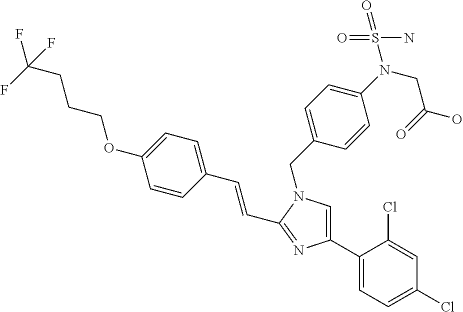 | {[4-(4-(2,4-Dichloro- phenyl)-2-{2 [4-(4,4,4-trifluoro-butoxy) phenyl]-(E)-vinyl}- imidazol-1- ylmethyl)-phenyl]-N- sulfonyl-amino}-acetic acid |
| 378 | 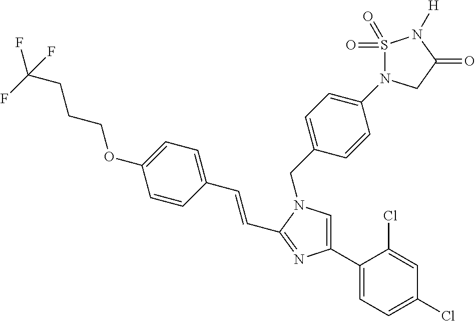 | 5-[4-(4-(2,4-Dichloro- phenyl)-2-{2-[4-(4,4,4- trifluoro-butoxy)-phenyl]- (E)-vinyl}-imidazol-1- ylmethyl)-phenyl]-1,2,5- thiadiazolidin-3-one-1,1- dioxide |
| 379 | 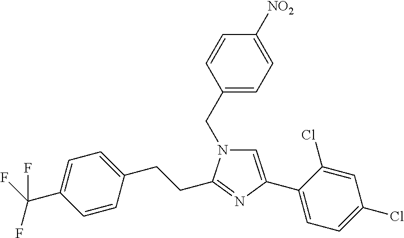 | 4-(2,4-Dichloro-phenyl)- 1-(4-nitro-benzyl)-2-[2-(4- trifluoromethyl-phenyl)- ethyl]-1H-imidazole |
| 380 | 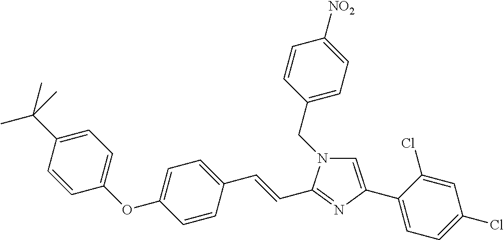 | 2-{2-[4-(4-tert-Butyl- phenoxy) phenyl]-(E)-vinyl}-4-(2,4- dichloro phenyl)-1-(4-nitro-benzyl)- 1H-imidazole |
| 381 | 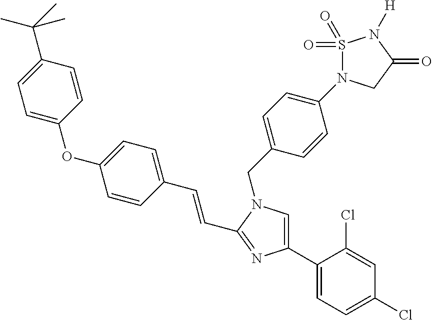 | 5-{4-[2-{2-[4-(4-tert-Butyl phenoxy)-phenyl]-(E)- vinyl}-4-(2,4 dichloro-phenyl)- imidazol-1 ylmethyl]-phenyl}-1,2,5- thiadiazolidin-3-one-1,1- dioxide |
| 382 | 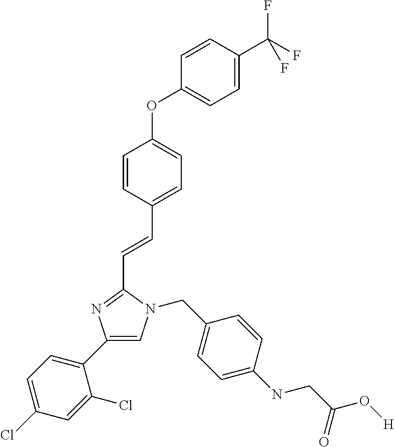 | [4-(4-(2,4-Dichloro- phenyl)-2-{2 [4-(4-trifluoromethyl- phenoxy) phenyl]-(E)-vinyl}- imidazol-1 ylmethyl)-phenylamino]- acetic acid |
| 383 | 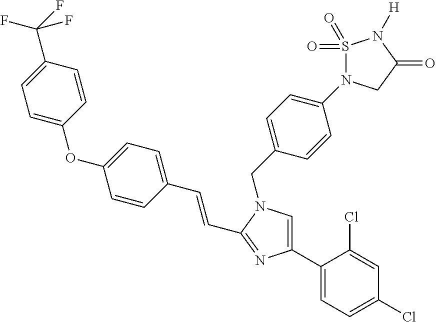 | 5-[4-(4-(2,4-Dichloro- phenyl)-2-{2-[4-(4- trifluoromethyl-phenoxy)- phenyl]-(E)-vinyl}- imidazol-1-ylmethyl)- phenyl]- 1,2,5]thiadiazolidin-3- one-1,1-dioxide |
| 384 | 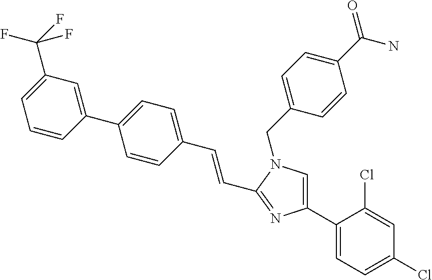 | 4-{4-(2,4-Dichloro- phenyl)-2-[2-(3′- trifluoromethyl-biphenyl- 4-yl)-(E)-vinyl]-imidazol- 1-ylmethyl}-benzamide |
| 385 | 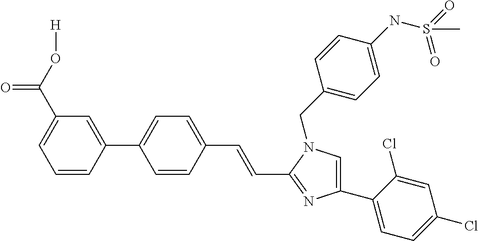 | 4′-{2-[4-(2,4-Dichloro- phenyl)-1-(4- methanesulfonylamino- benzyl)-1H-imidazol-2- yl]-(E)-vinyl}-biphenyl-3- carboxylic acid |
| 386 | 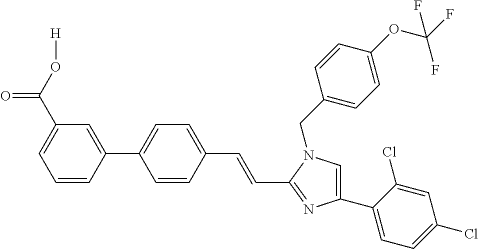 | 4′-{2-[4-(2,4-Dichloro- phenyl)-1-(4- trifluoromethoxy-benzyl)- 1H-imidazol-2-yl]-(E)- vinyl}-biphenyl-3- carboxylic acid |
| 387 | 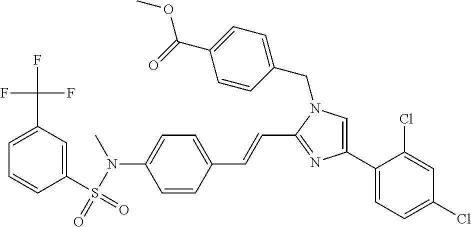 | 4-[4-(2,4-dichloro- phenyl)-2-(2-{4-[methyl- (3-trifluoromethyl- benzenesulfonyl)- amino]-phenyl}-(E)- vinyl)-imidazol-1- ylmethyl]-benzoic acid methyl ester |
| 388 | 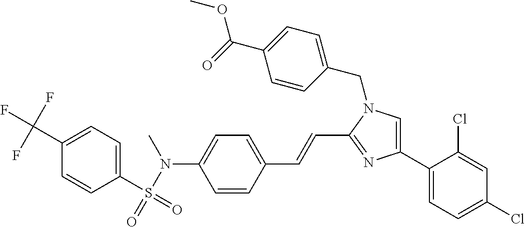 | 4-[4-(2,4-dichloro- phenyl)-2-(2-{4-[methyl- (4-trifluoromethyl- benzenesulfonyl)- amino]-phenyl}-(E)- vinyl)-imidazol-1- ylmethyl]-benzoic acid methyl ester |
| 389 | 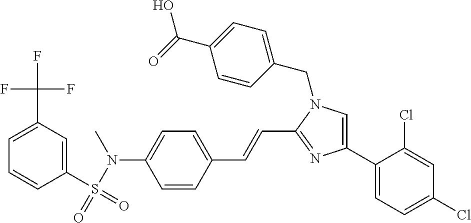 | 4-[4-(2,4-dichloro- phenyl)-2-(2-{4-[methyl- (3-trifluoromethyl- benzenesulfonyl)- amino]-phenyl}-(E)- vinyl)-imidazol-1- ylmethyl]-benzoic acid |
| 390 | 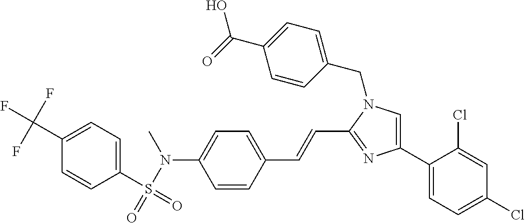 | 4-[4-(2,4-dichloro- phenyl)-2-(2-{4-[methyl- (4-trifluoromethyl- benzenesulfonyl- amino]-phenyl}-(E)- vinyl)-imidazol-1- ylmethyl]-benzoic acid |
| 391 | 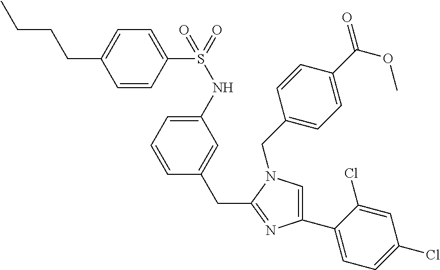 | 4-[2-[3-(4-butyl- benzenesulfonylamino)- benzyl]-4-(2,4-dichloro- phenyl)-imidazol-1- ylmethyl]-benzoic acid methyl ester |
| 392 | 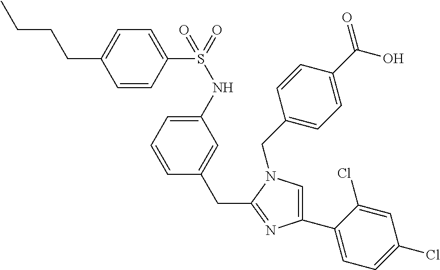 | 4-[2-[3-(4-Butyl- benzenesulfonylamino)- benzyl]-4-(2,4-dichloro- phenyl)-imidazol-1- ylmethyl]-benzoic acid |
| 393 | 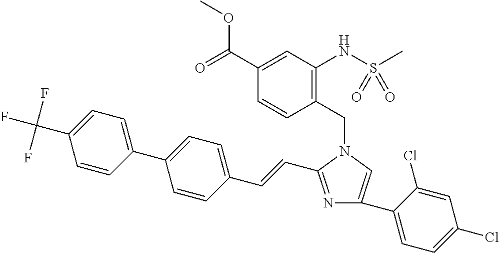 | 4-{4-(2,4-dichloro- phenyl)-2-[2-(4′- trifluoromethyl-biphenyl- 4-yl)-(E)-vinyl]-imidazol- 1-ylmethyl}-3- methanesulfonylamino- benzoic acid methyl ester |
| 394 | 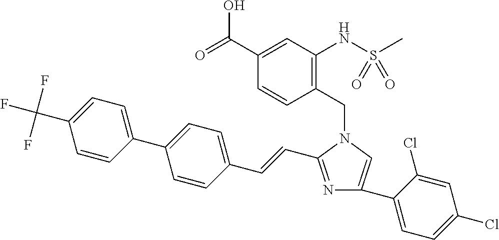 | 4-{4-(2,4-dichloro- phenyl)-2-[2-(4′- trifluoromethyl-biphenyl- 4-yl)-(E)-vinyl]-imidazol- 1-ylmethyl}-3- methanesulfonylamino- benzoic acid |
| 395 | 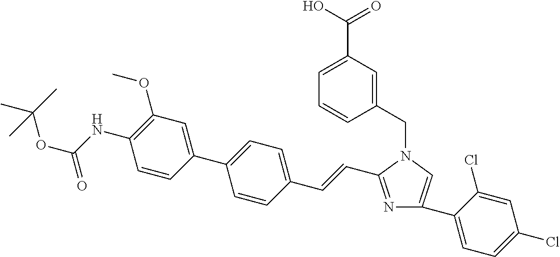 | 3-[2-[2-(4′-tert- butoxycarbonylamino-3′- methoxy-biphenyl-4-yl)- (E)-vinyl]-4-(2,4-dichloro- phenyl)-imidazol-1- ylmethyl]-benzoic acid |
| 396 | 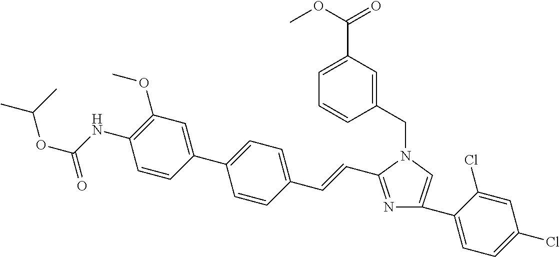 | 3-{4-(2,4-dichloro- phenyl)-2-[2-(4′- isopropoxycarbonylamino- 3′-methoxy-biphenyl-4- yl)-(E)-vinyl]-imidazol-1- ylmethyl}-benzoic acid methyl ester |
| 397 | 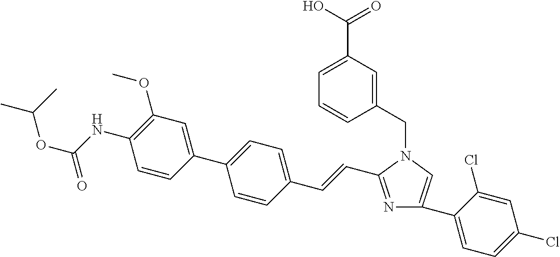 | 3-{4-(2,4-dichloro- phenyl)-2-[2-(4′- isopropoxycarbonylamino- 3′-methoxy-biphenyl-4- yl)-(E)-vinyl]-imidazol-1- ylmethyl}-benzoic acid |
| 398 | 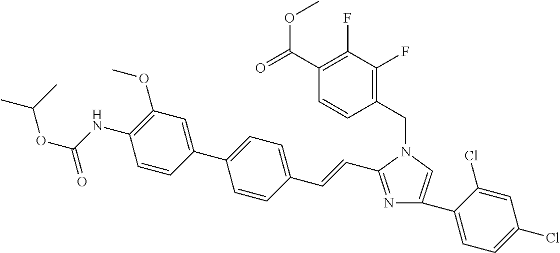 | 4-{4-(2,4-dichloro- phenyl)-2-[2-(4′- isopropoxycarbonylamino- 3′-methoxy-biphenyl-4- yl)-(E)-vinyl]-imidazol-1- ylmethyl}-2,3-difluoro- benzoic acid methyl ester |
| 399 | 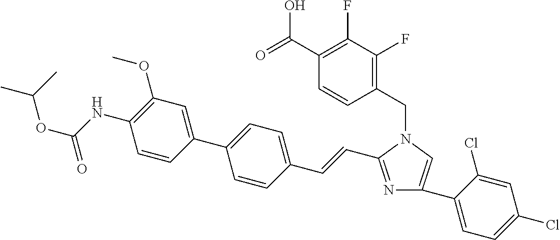 | 4-{4-(2,4-dichloro- phenyl)-2-[2-(4′- isopropoxycarbonylamino- 3′-methoxy-biphenyl-4- yl)-(E)-vinyl]-imidazol-1- ylmethyl}-2,3-difluoro- benzoic acid |
| 400 | 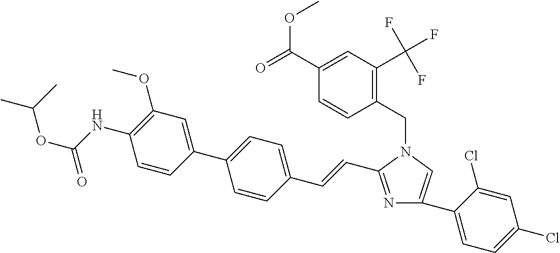 | 4-{4-(2,4-dichloro- phenyl)-2-[2-(4′- isopropoxycarbonylamino- 3′-methoxy-biphenyl-4- yl)-(E)-vinyl]-imidazol-1- ylmethyl}-3- trifluoromethyl-benzoic acid methyl ester |
| 401 | 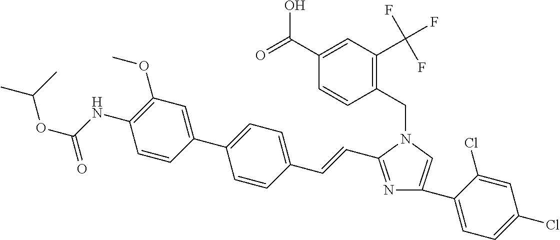 | 4-{4-(2,4-dichloro- phenyl)-2-[2-(4′- isopropoxycarbonylamino- 3′-methoxy-biphenyl-4- yl)-(E)-vinyl]-imidazol-1- ylmethyl}-3- trifluoromethyl--benzoic acid |
| 402 | 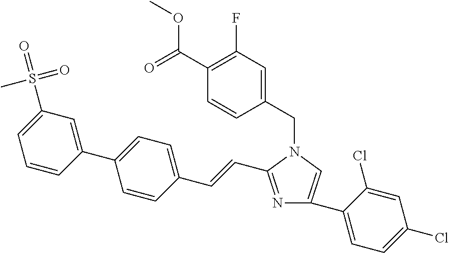 | 4-{4-(2,4-dichloro- phenyl)-2-[2-(3′- methanesulfonyl- biphenyl-4-yl)-(E)-vinyl]- imidazol-1-ylmethyl}-2- fluoro-benzoic acid methyl ester |
| 403 | 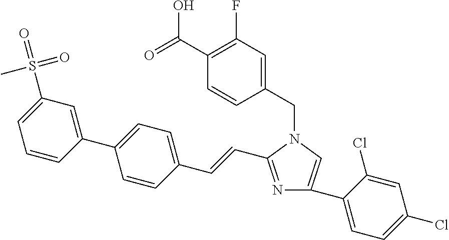 | 4-{4-(2,4-dichloro- phenyl)-2-[2-(3′- methanesulfonyl- biphenyl-4-yl)-(E)-vinyl]- imidazol-1-ylmethyl}-2- fluoro-benzoic acid |
| 404 | 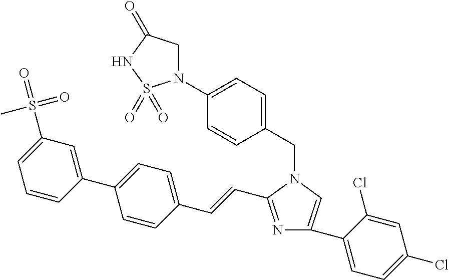 | 5-(4-{4-(2,4-dichloro- phenyl)-2-[2-(3′- methanesulfonyl- biphenyl-4-yl)-(E)-vinyl]- imidazol-1-ylmethyl}- phenyl)-1,2,5- thiadiazolidine-3-one- 1,1-dioxide |
| 405 | 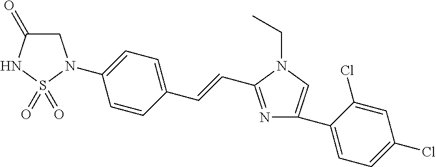 | 5-(4-{2-[4-(2,4-dichloro- phenyl)-1-ethyl-1H- imidazol-2-yl]-(E)-vinyl}- phenyl)-1,2,5- thiadiazolidine-3-one- 1,1-dioxide |
| 406 | 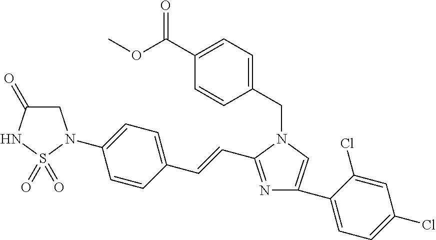 | 4-(4-(2,4-dichloro- phenyl)-2-{2-[4-(1,1,4- trioxo-1,2,5- thiadiazolidin-2-yl)- phenyl]-(E)-vinyl}- imidazol-1-ylmethyl)- benzoic acid methyl ester |
| 407 | 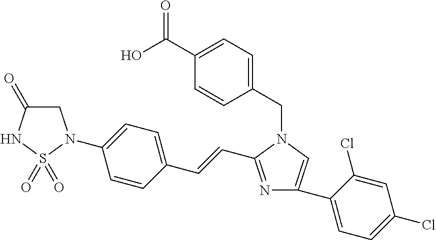 | 4-(4-(2,4-dichloro- phenyl)-2-{2-[4-(1,1,4- trioxo-1,2,5- thiadiazolidin-2-yl)- phenyl]-(E)-vinyl}- imidazol-1-ylmethyl)- benzoic acid |
| 408 | 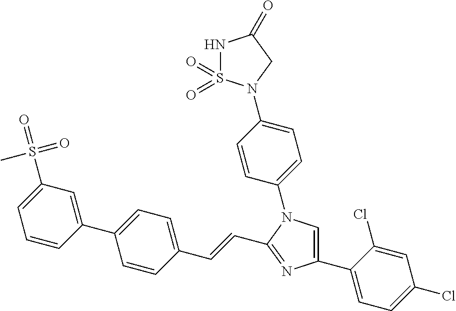 | 5-(4-{4-(2,4-dichloro- phenyl)-2-[2-(3′- methanesulfonyl- biphenyl-4-yl)-(E)-vinyl]- imidazol-1-yl}-phenyl)- 1,2,5-thiadiazolidine-3- one-1,1-dioxide |
| 409 | 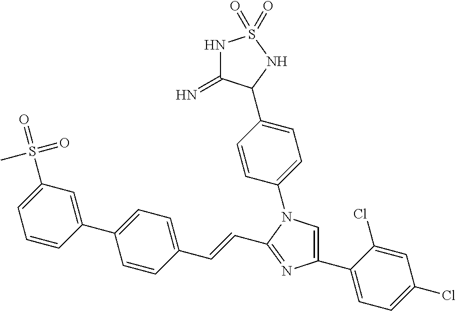 | (±)-4-(4-{4-(2,4-dichloro- phenyl)-2-[2-(3′- methanesulfonyl- biphenyl-4-yl)-(E)-vinyl]- imidazol-1-yl}-phenyl)- 1,1-dioxo-1,2,5- thiadiazolidin-3- ylideneamine |
| 410 | 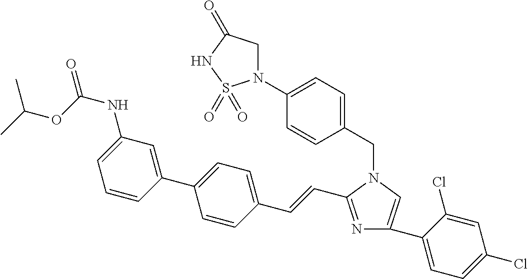 | [4′-(2-{4-(2,4-dichloro- phenyl)-1-[4-(1,1,4- trioxo-1,2,5- thiadiazolidin-2-yl)- benzyl]-1H-imidazol-2- yl}-(E)-vinyl)-biphenyl-3- yl]-carbamic acid isopropyl ester |
| 411 | 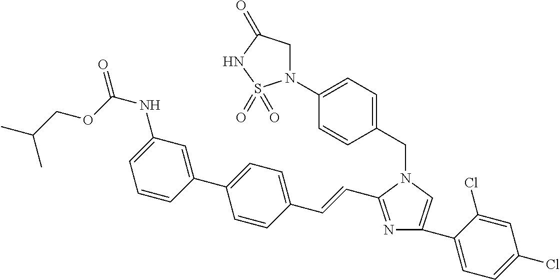 | [4′-(2-{4-(2,4-dichloro- phenyl)-1-[4-(1,1,4- trioxo-1,2,5- thiadiazolidin-2-yl)- benzyl]-1H-imidazol-2- yl}-(E)-vinyl)-biphenyl-3- yl]-carbamic acid isobutyl ester |
| 412 | 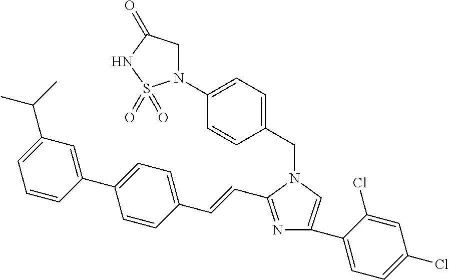 | 5-(4-{4-(2,4-dichloro- phenyl)-2-[2-(3′- isopropyl-biphenyl-4-yl)- (E)-vinyl]-imidazol-1- ylmethyl}-phenyl)-1,2,5- thiadiazolidine-3-one- 1,1-dioxide |
| 413 | 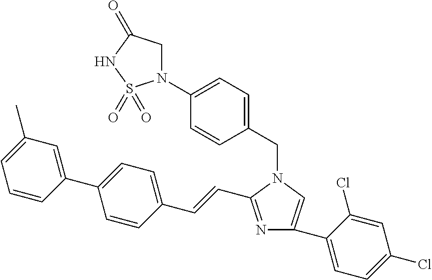 | 5-(4-{4-(2,4-dichloro- phenyl)-2-[2-(3′-methyl- biphenyl-4-yl)-(E)-vinyl]- imidazol-1-ylmethyl}- phenyl)-1,2,5- thiadiazolidine-3-one- 1,1-dioxide |
| 414 | 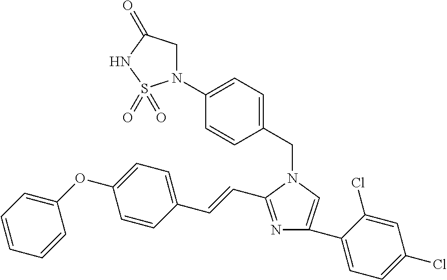 | 5-(4-{4-(2,4-dichloro- phenyl)-2-[2-(4-phenoxy- phenyl)-(E)-vinyl]- imidazol-1-ylmethyl}- phenyl)-1,2,5- thiadiazolidine-3-one- 1,1-dioxide |
| 415 | 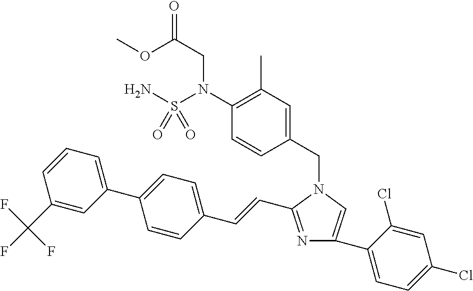 | (4-{4-(2,4-dichloro- phenyl)-2-[2-(3′- trifluoromethyl-biphenyl- 4-yl)-(E)-vinyl]-imidazol- 1-ylmethyl}-2-methyl- phenylaminosulfonamido)- acetic acid methyl ester |
| 416 | 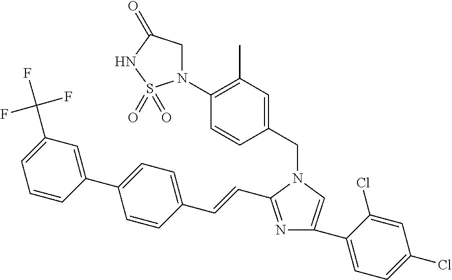 | 5-(4-{4-(2,4-dichloro- phenyl)-2-[2-(3′- trifluoromethyl-biphenyl- 4-yl)-(E)-vinyl]-imidazol- 1-ylmethyl}-2-methyl- phenyl)-1,2,5- thiadiazolidine-3-one- 1,1-dioxide |
| 417 | 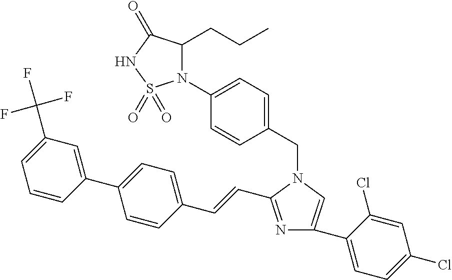 | (±)-5-(4-{4-(2,4-dichloro- phenyl)-2-[2-(3′- trifluoromethyl-biphenyl- 4-yl)-(E)-vinyl]-imidazol- 1-ylmethyl}-phenyl)-4- propyl-1,2,5- thiadiazolidine-3-one- 1,1-dioxide |
| 418 | 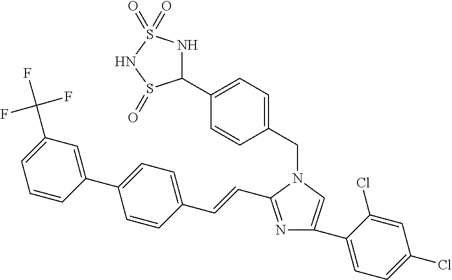 | (±)-4-(4-{4-(2,4-dichloro- phenyl)-2-[2-(3′- trifluoromethyl-biphenyl- 4-yl)-(E)-vinyl]-imidazol- 1-ylmethyl}-phenyl)- 1,2,5-thiadiazolidine-3- one-1,1-dioxide |
| 419 | 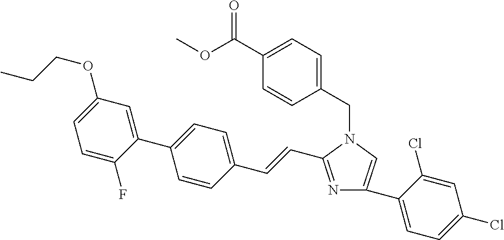 | 4-{4-(2,4-dichloro- phenyl)-2-[2-(2′-fluoro-5′- propoxy-biphenyl-4-yl)- (E)-vinyl]-imidazol-1- ylmethyl}-benzoic acid methyl ester |
| 420 | 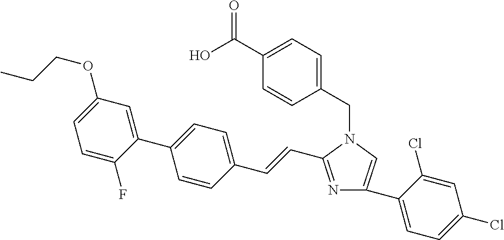 | 4-{4-(2,4-dichloro- phenyl)-2-[2-(2′-fluoro-5′- propoxy-biphenyl-4-yl)- (E)-vinyl]-imidazol-1- ylmethyl}-benzoic acid |
| 421 | 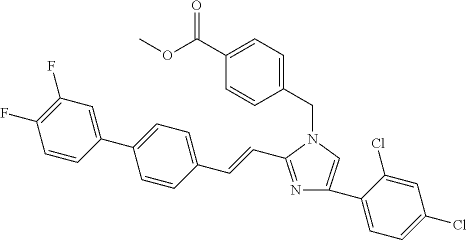 | 4-{4-(2,4-dichloro- phenyl)-2-[2-(3′,4′- difluoro-biphenyl-4-yl)- (E)-vinyl]-imidazol-1- ylmethyl}-benzoic acid methyl ester |
| 422 | 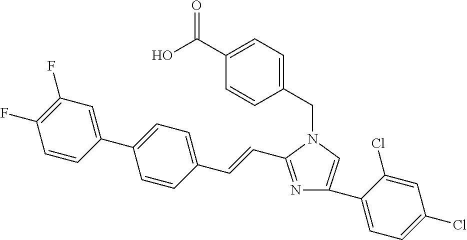 | 4-{4-(2,4-Dichloro- phenyl)-2-[2-(3′,4′- difluoro-biphenyl-4-yl)- (E)-vinyl]-imidazol-1- ylmethyl}-benzoic acid |
| 423 | 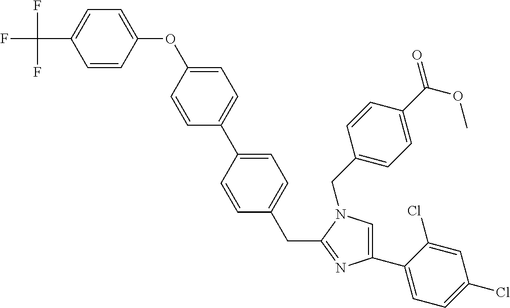 | 4-{4-(2,4-dichloro- phenyl)-2-[4′-(4- trifluoromethyl-phenoxy)- biphenyl-4-ylmethyl]- imidazol-1-ylmethyl}- benzoic acid methyl ester |
| 424 | 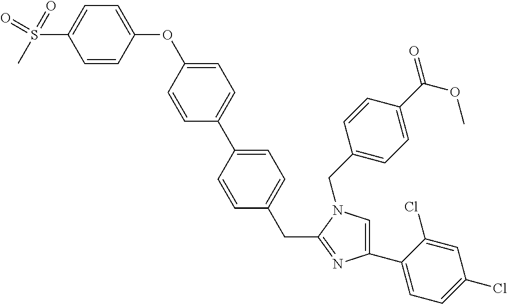 | 4-{4-(2,4-dichloro- phenyl)-2-[4′-(4- methanesulfonyl- phenoxy)-biphenyl-4- ylmethyl]-imidazol-1- ylmethyl}-benzoic acid methyl ester |
| 425 | 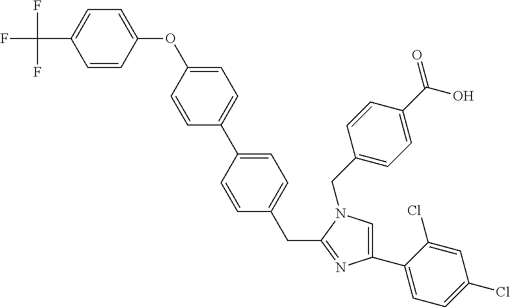 | 4-{4-(2,4-dichloro- phenyl)-2-[4′-(4- trifluoromethyl-phenoxy)- biphenyl-4-ylmethyl]- imidazol-1-ylmethyl}- benzoic acid |
| 426 | 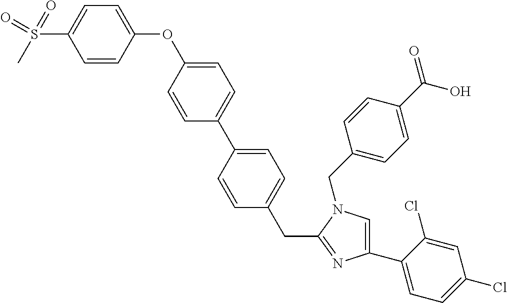 | 4-{4-(2,4-dichloro- phenyl)-2-[4′-(4- methanesulfonyl- phenoxy)-biphenyl-4- ylmethyl]-imidazol-1- ylmethyl}-benzoic acid |
| 427 | 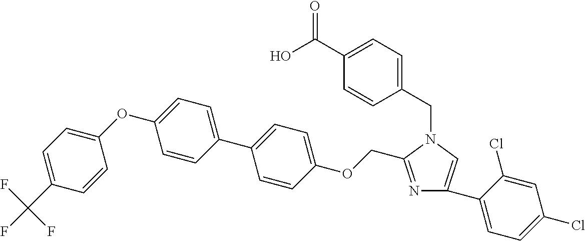 | 4-{4-(2,4-dichloro- phenyl)-2-[4′-(4- trifluoromethyl-phenoxy)- biphenyl-4-yloxymethyl]- imidazol-1-ylmethyl}- benzoic acid |
| 428 | 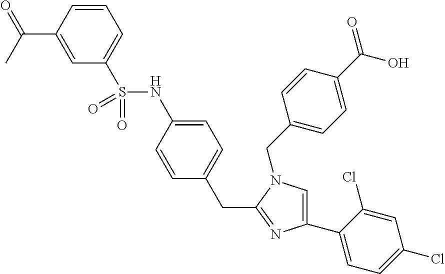 | 4-[2-[4-(3-acetyl- benzenesulfonylamino)- benzyl]-4-(2,4-dichloro- phenyl)-imidazol-1- ylmethyl]-benzoic acid |
| 429 | 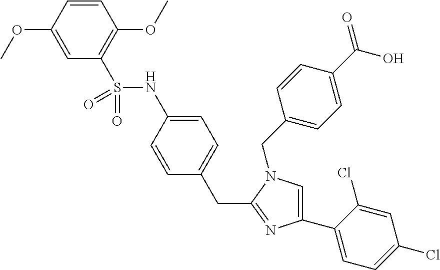 | 4-{4-(2,4-dichloro- phenyl)-2-[4-(2,5- dimethoxy- benzenesulfonylamino)- benzyl]-imidazol-1- ylmethyl}-benzoic acid |
| 430 | 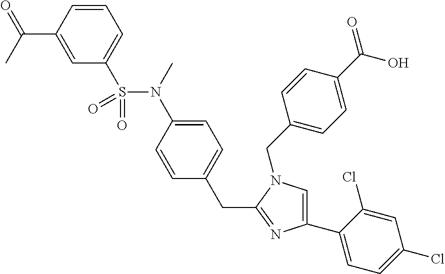 | 4-[2-{4-[(3-acetyl- benzenesulfonyl)-methyl- amino]-benzyl}-4-(2,4- dichloro-phenyl)- imidazol-1-ylmethyl]- benzoic acid |
| 431 | 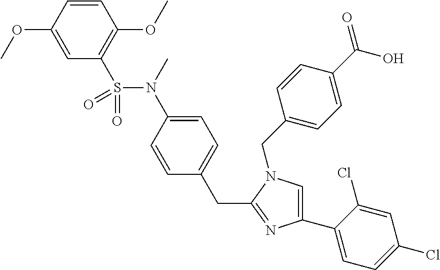 | 4-(4-(2,4-dichloro- phenyl)-2-{4-[(2,5- dimethoxy- benzenesulfonyl)-methyl- amino]-benzyl}-imidazol- 1-ylmethyl)benzoic acid |
| 432 | 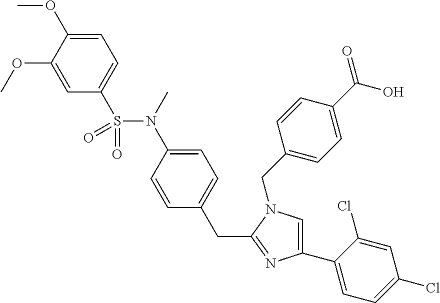 | 4-(4-(2,4-dichloro- phenyl)-2-{4-[(3,4- dimethyoxy- benzenesulfonyl)-methyl- amino]-benzyl}-imidazol- 1-ylmethyl)-benzoic acid |
| 433 | 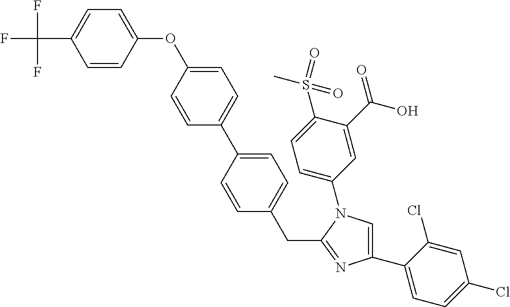 | 5-{4-(2,4-dichloro- phenyl)-2-[4′-(4- trifluoromethyl-phenoxy)- biphenyl-4-ylmethyl]- imidazol-1-yl}-2- methanesulfonyl-benzoic acid |
| 434 | 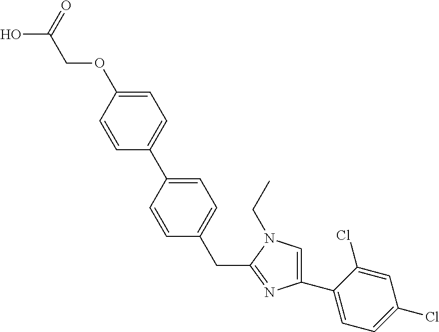 | {4′-[4-(2,4-dichloro- phenyl)-1-ethyl-1H- imidazol-2-ylmethyl]- biphenyl-4-yloxy}-acetic acid |
| 435 | 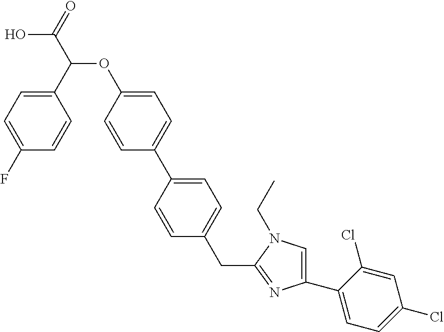 | {4′-[4-(2,4-dichloro- phenyl)-1-ethyl-1H- imidazol-2-ylmethyl]- biphenyl-4-yloxy}-(4- fluoro-phenyl)-acetic acid |
| 436 | 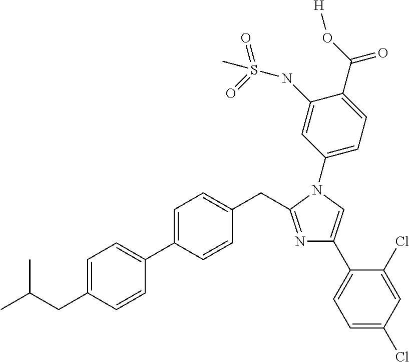 | 4-[2-(4′-Isobutyl- biphenyl-4-ylmethyl)-4- (2,4-dichloro-phenyl) imidazol-1-yl]-2- methanesulfonylamino- benzoic acid |
| 437 | 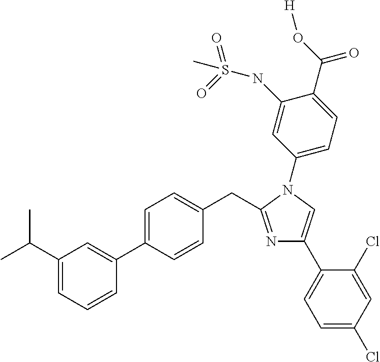 | 4-[2-(3′-Isopropyl- biphenyl-4-ylmethyl)-4- (2,4-dichloro-phenyl)- imidazol-1-yl]-2- methanesulfonylamino- benzoic acid |
| 438 | 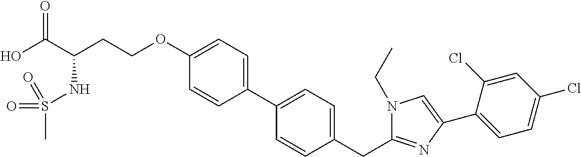 | 4-{4′-[4-(2,4-Dichloro- phenyl)-1-ethyl-1H- imidazol-2-ylmethyl]- biphenyl-4-yloxy}-2-(S)- methanesulfonylamino- butyric acid |
| 439 | 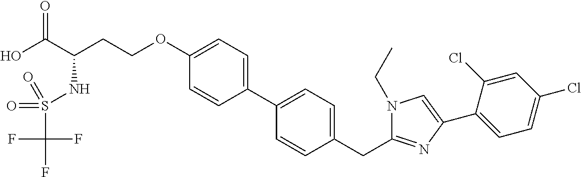 | 4-{4′-[4-(2,4-Dichloro- phenyl)-1-ethyl-1H- imidazol-2-ylmethyl]- biphenyl-4-yloxy}-2-(S)- trifluoromethanesulfonyl- amino-butyric acid |
| 440 | 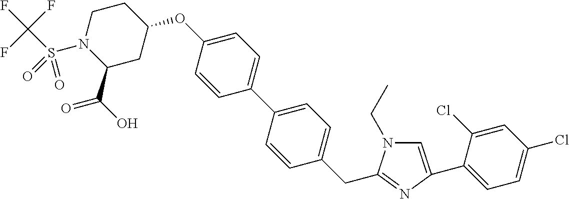 | 4(S)-{4′-[4-(2,4-Dichloro- phenyl)-1-ethyl-1H- imidazol-2-ylmethyl]- biphenyl-4-yloxy}-1- trifluoromethanesulfonyl- piperidine-2-(S)- carboxylic acid |
| 441 | 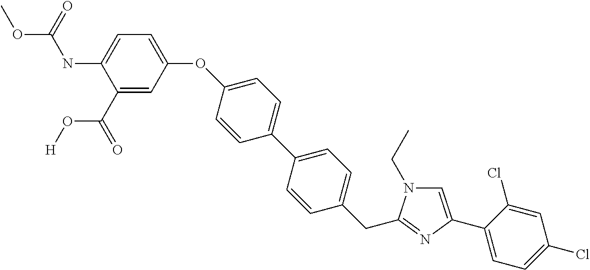 | 5-{4′-[4-(2,4-Dichloro- phenyl)-1-ethyl-1H- imidazol-2-ylmethyl]- biphenyl-4-yloxy}-2- methoxycarbonylamino- benzoic acid |
| 442 | 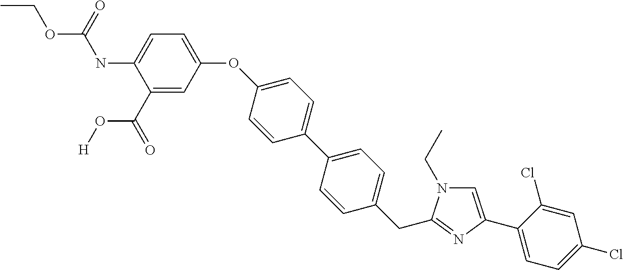 | 5-{4′-[4-(2,4-Dichloro- phenyl)-1-ethyl-1H- imidazol-2-ylmethyl]- biphenyl-4-yloxy}-2- ethoxycarbonylamino- benzoic acid |
| 443 | 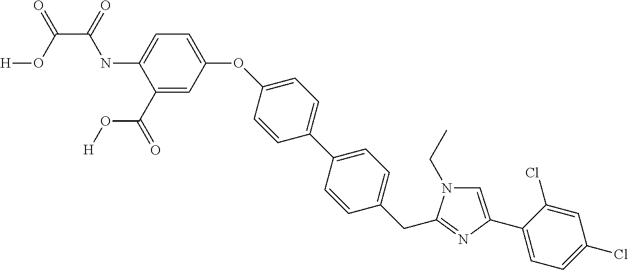 | 5-{4′-[4-(2,4-Dichloro- phenyl)-1-ethyl-1H- imidazol-2-ylmethyl]- biphenyl-4-yloxy}-2- (oxalyl-amino)-benzoic acid |
| 444 | 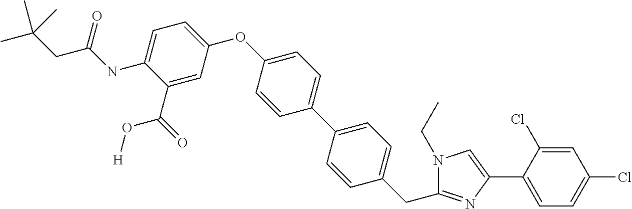 | 5-{4′-[4-(2,4-Dichloro- phenyl)-1-ethyl-1H- imidazol-2-ylmethyl]- biphenyl-4-yloxy}-2-(3,3- dimethyl-butyrylamino)- benzoic acid |
| 445 | 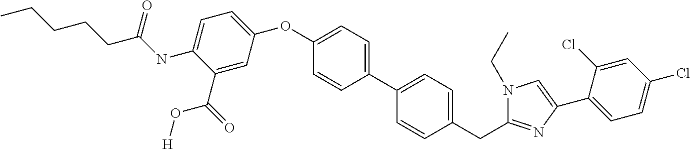 | 5-{4′-[4-(2,4-Dichloro- phenyl)-1-ethyl-1H- imidazol-2-ylmethyl]- biphenyl-4-yloxy}-2- hexanoylamino-benzoic acid |

Incomplete valences for heteroatoms such as oxygen and nitrogen in the chemical structures listed in Table 1 are assumed to be completed by hydrogen.

In another aspect, the present invention comprises a pharmaceutical composition comprising the compound of Formula (I) and one or more pharmaceutically acceptable carriers, excipients, or diluents.

As used herein, the term “lower” refers to a group having between one and six carbons.

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “alkyl” as used herein include, but are not limited to, methyl, n-butyl, t-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ atoms. Examples of “alkylene” as used herein include, but are not limited to, methylene, ethylene, and the like.

As used herein, the term “alkyline” refers to a straight or branched chain trivalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “alkyline” as used herein include, but are not limited to, methine, ethyline, and the like.

As used herein, the term “alkenyl” refers to a hydrocarbon radical having from two to ten carbons and at least one carbon-carbon double bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ atoms.

As used herein, the term “alkenylene” refers to a straight or branched chain divalent hydrocarbon radical having from two to ten carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ atoms.

As used herein, the term “alkynylene” refers to a straight or branched chain divalent hydrocarbon radical having from two to ten carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ atoms. Examples of “alkynylene” as used herein include, but are not limited to, ethyne-1,2-diyl, propyne-1,3-diyl, and the like.

As used herein, “cycloalkyl” refers to an alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “cycloalkyline” refers to an non-aromatic alicyclic trivalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkyline” as used herein include, but are not limited to, cyclopropyl-1,1,2-triyl, cyclohexyl-1,3,4-triyl, and the like.

As used herein, the term “heterocyclic” or the term “heterocyclyl” refers to a three to twelve-membered heterocyclic ring optionally possessing one or more degrees of unsaturation, containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, 1,4-dioxane, 1,3-dioxane, piperidine, pyrrolidine, morpholine, piperazine, and the like.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

As used herein, the term “heterocyclyline” refers to a three to twelve-membered heterocyclic ring triradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclyline” include, but are not limited to, tetrahydrofuran-2,4,5-triyl, morpholine-2,3,4-triyl, pyran-2,4,5-triyl, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

As used herein, the term “aryline” refers to a benzene ring triradical or to a benzene ring system triradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, trialkylsilylalkyloxyalkyl, silyl optionally substituted by alkoxy, alkyl, or aryl, nitro, cyano, halogen, or lower perfluoroalkyl, multiple degrees of substitution being allowed. Examples of “aryline” include, but are not limited to, benzene-1,2,4-triyl, naphthalene-1,4,8-triyl, and the like.

As used herein, the term “heteroaryl” refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “heteroaryline” refers to a five- to seven-membered aromatic ring triradical, or to a polycyclic heterocyclic aromatic ring triradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkylsulfonylamino optionally substituted by alkyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroaryline” used herein are furan-2,4,5-triyl, thiophene-2,3,4-triyl, and the like.

As used herein, the term “fused cycloalkylaryl” refers to one or more cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl,

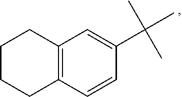

and the like.

As used herein, the term “fused cycloalkylarylene” refers to a fused cycloalkylaryl, wherein the aryl group is divalent. Examples include

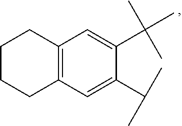

and the like.

As used herein, the term “fused arylcycloalkyl” refers to one or more aryl groups fused to a cycloalkyl group, the cycloalkyl and aryl groups having two atoms in common, and wherein the cycloalkyl group is the point of substitution. Examples of “fused arylcycloalkyl” used herein include 1-indanyl, 2-indanyl, 9-fluorenyl, 1-(1,2,3,4-tetrahydronaphthyl),

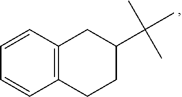

and the like.

As used herein, the term “fused arylcycloalkylene” refers to a fused arylcycloalkyl, wherein the cycloalkyl group is divalent. Examples include 9,1-fluorenylene,

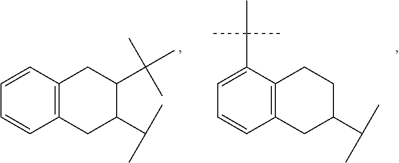

and the like.

As used herein, the term “fused heterocyclylaryl” refers to one or more heterocyclyl groups fused to an aryl group, the aryl and heterocyclyl groups having two atoms in common, and wherein the aryl group is the point of substitution. Examples of “fused heterocyclylaryl” used herein include 3,4-methylenedioxy-1-phenyl,

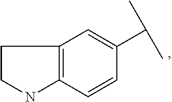

and the like

As used herein, the term “fused heterocyclylarylene” refers to a fused heterocyclylaryl, wherein the aryl group is divalent. Examples include

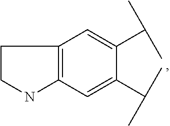

and the like.

As used herein, the term “fused arylheterocyclyl” refers to one or more aryl groups fused to a heterocyclyl group, the heterocyclyl and aryl groups having two atoms in common, and wherein the heterocyclyl group is the point of substitution. Examples of “fused arylheterocyclyl” used herein include 2-(1,3-benzodioxolyl),

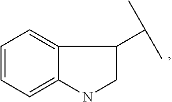

and the like.

As used herein, the term “fused arylheterocyclylene” refers to a fused arylheterocyclyl, wherein the heterocyclyl group is divalent. Examples include

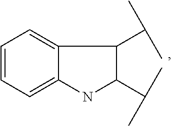

and the like.

As used herein, the term “fused cycloalkylheteroaryl” refers to one or more cycloalkyl groups fused to a heteroaryl group, the heteroaryl and cycloalkyl groups having two atoms in common, and wherein the heteroaryl group is the point of substitution. Examples of “fused cycloalkylheteroaryl” used herein include 5-aza-6-indanyl,

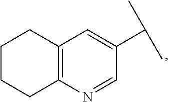

and the like.

As used herein, the term “fused cycloalkylheteroarylene” refers to a fused cycloalkylheteroaryl, wherein the heteroaryl group is divalent. Examples include

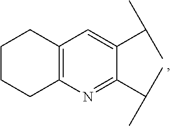

and the like.

As used herein, the term “fused heteroarylcycloalkyl” refers to one or more heteroaryl groups fused to a cycloalkyl group, the cycloalkyl and heteroaryl groups having two atoms in common, and wherein the cycloalkyl group is the point of substitution. Examples of “fused heteroarylcycloalkyl” used herein include 5-aza-1-indanyl,

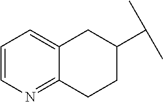

and the like.

As used herein, the term “fused heteroarylcycloalkylene” refers to a fused heteroarylcycloalkyl, wherein the cycloalkyl group is divalent. Examples include

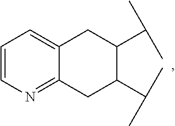

and the like.

As used herein, the term “fused heterocyclylheteroaryl” refers to one or more heterocyclyl groups fused to a heteroaryl group, the heteroaryl and heterocyclyl groups having two atoms in common, and wherein the heteroaryl group is the point of substitution. Examples of “fused heterocyclylheteroaryl” used herein include 1,2,3,4-tetrahydro-beta-carbolin-8-yl,

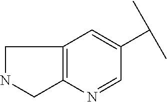

and the like.

As used herein, the term “fused heterocyclylheteroarylene” refers to a fused heterocyclylheteroaryl, wherein the heteroaryl group is divalent. Examples include

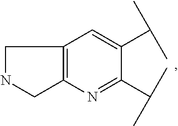

and the like.

As used herein, the term “fused heteroarylheterocyclyl” refers to one or more heteroaryl groups fused to a heterocyclyl group, the heterocyclyl and heteroaryl groups having two atoms in common, and wherein the heterocyclyl group is the point of substitution. Examples of “fused heteroarylheterocyclyl” used herein include -5-aza-2,3-dihydrobenzofuran-2-yl,

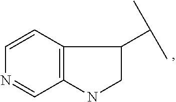

and the like.

As used herein, the term “fused heteroarylheterocyclylene” refers to a fused heteroarylheterocyclyl, wherein the heterocyclyl group is divalent. Examples include

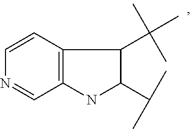

and the like.

As used herein, the term “acid isostere” refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, 5-hydroxy-4H-pyran-4-on-2-yl, 1,2,5-thiadiazolidin-3-one-1,1-dioxide-4-yl, 1,2-5-thiadiazolidin-3-one-1,1-dioxide-5-yl, 1,2,5-thiadiazolidin-3-one-1,1-dioxide-5-yl having substituents at the 2 and/or 4 position; or —N-acyl-alkylsulfonamide.

As used herein, the term “side chain of a natural or non-natural amino acid” refers to the group “R” in a substance of formula HO₂C—CH(R)—NH₂. Examples of such substances bearing a group “R” include but are not limited to alanine, asparigine, arginine, aspartic acid, cystine, cysteine, glutamic acid, histidine, isoleucine, leucine, lysine, methionine, phenylalanine, serine, threonine, tryptophan, tyrosine, valine, alpha-aminoadipic acid, alpha-aminobutyric acid, norleucine, 3,4-dihydroxyphenylalanine, homoserine, and ornithine. Where such groups “R” bear carboxyl, hydroxyl, or amino functional groups, such functional groups may be protected. In addition, where groups “R” bear a sulfhydryl group, such a group may be protected in a form such as but not limited to a tert-butyl thioether, a benzyl thioether, or an alkanoyl thioester.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfenyl” refers to the group RₐS(O)—, where Rₐ is alkyl.

As used herein, the term “alkenylsulfenyl” refers to the group RₐS(O)—, where Rₐ is alkenyl.

As used herein, the term “alkynylsulfenyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “aroyloxy” refers to the group RₐC(O)O—, where Rₐ is aryl.

As used herein, the term “heteroaroyloxy” refers to the group RₐC(O)O—, where Rₐ is heteroaryl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” shall include iodine, bromine, chlorine and fluorine.

As used herein, the term “mercapto” shall refer to the substituent —SH.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)₂—.

The compounds can be prepared according to the following reaction Schemes (in which variables are as defined before or are defined) using readily available starting materials, and reagents. In these reactions, it is also possible to make use of variants which are themselves known to those of ordinary skill in this art, but are not mentioned in greater detail.

The present invention also provides a method for the synthesis of compounds useful as intermediates in the preparation of compounds of Formula (I) along with methods for the preparation of compounds of Formula (I). Unless otherwise specified, structural variables are as defined for Formula (I).

An unsaturated carboxylic acid (Scheme 1) can be reacted with aryl acyl bromides in the presence of base such as DIEA, triethyl amine, or DBU in a polar solvents such as THF, or DMF to afford intermediate keto-ester (2), which can be treated with ammonium acetate in acetic acid at temperatures ranging from 60-120° C., which leads to the corresponding mixture of oxazole (W═O) and imidazole (W═N) (3) (Strzybny, P. P. E., van Es, T.; Backeberg, O. G., J. Org. Chem. 1963, 25, 1151). The ratio of oxazole and imidazole may vary depending on the substitution and reaction conditions and the two compounds were separated through silica gel column. Alternatively other conditions may also be employed for cyclization of keto-esters (2), such as BF₃/Et₂O, methanolic ammonia, at temperatures ranging from room temperature to 120° C.

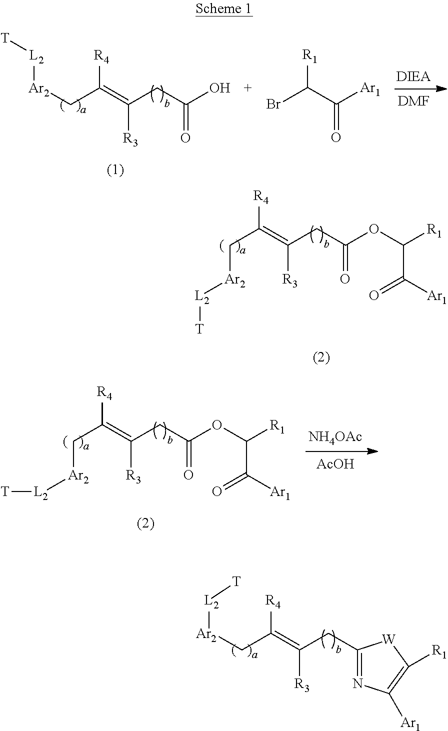

In another embodiment, a bromo or iodo aryl compound (4) (Scheme 2) can be subjected to palladium catalyzed coupling (Syn. Commu. 1981, 11, 513-574) with an optionally substituted heteteroaryl or aryl boronic acid. Ar₃ is a group such as but not limited to a heteroaryl or aryl group. Typical conditions used to carry out the coupling reaction include the use of boronic acid or ester as the coupling partner, a palladium catalyst (2 to 20 mole %) such as Pd(PPh₃)₄ or [1,1-bis(diphenylphosphino)-ferrocene]dichloro-palladium (II) and base such as potassium carbonate, sodium carbonate, barium hydroxide, potassium phosphate or triethyl amine in a suitable solvent such as aqueous dimethoxyethane, THF, acetone, DMF or toluene at temperatures ranging from 25° C. to 125° C. In this instance, Ar₃ is a group such as, but not limited to, an aryl or heteroaryl group.

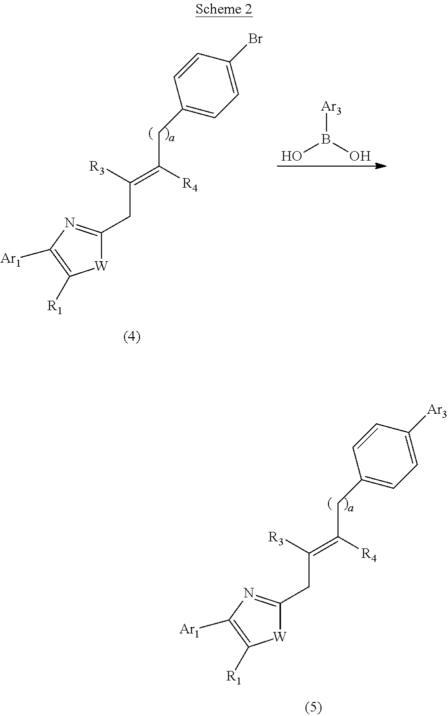

In another embodiment (Scheme 3), the O-alky, or O-aryl group in compound (5a) can be dealkylated or dearylated using reagents such as boron tribromide or PhSMe, in a solvent such as dichloromethane or TFA, at temperatures ranging from -20° C. to room temperature to afford hydroxy biphenyls (6). In this instance, Ar₄ is a group such as, but not limited to, heteryarylene or arylene, and R₃₀ is a group such as, but not limited to, lower alkyl.

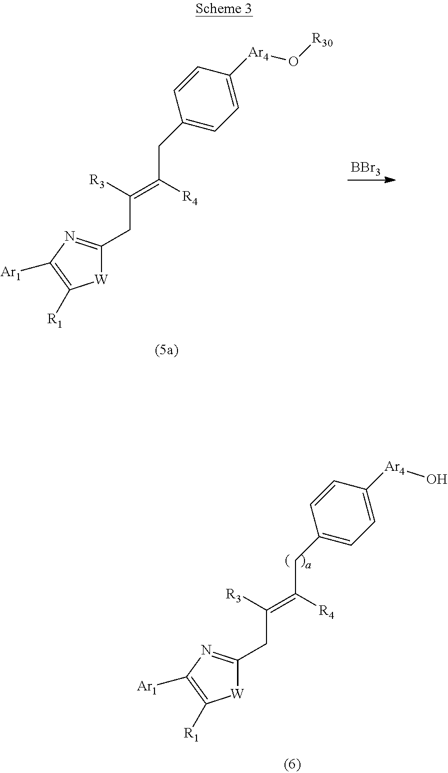

In Scheme 4, the biphenyl alcohols (6) were alkylated with bromo or chloro alkyl carboxylates [(Br or Cl)(CH₂)ₙ—CO₂—R₃₀] (where n=1 to 6) in the presence of base such as sodium hydride, potassium tert-butoxide, or potassium carbonate using DMF, THF, acetonitrile as the solvent at temperatures ranging from 50° C. to 100° C. Subsequent saponification of esters (7) with bases such as sodium hydroxide, lithium hydroxide in aqueous and organic solvents such as THF, methanol, at temperatures ranging from room temperature to 60° C. produces carboxylic acid (8). In this instance, R₃₀ is a group such as, but not limited to, lower alkyl. In this instance, Ar₄ is a group such as, but not limited to, an arylene or heteroarylene group.

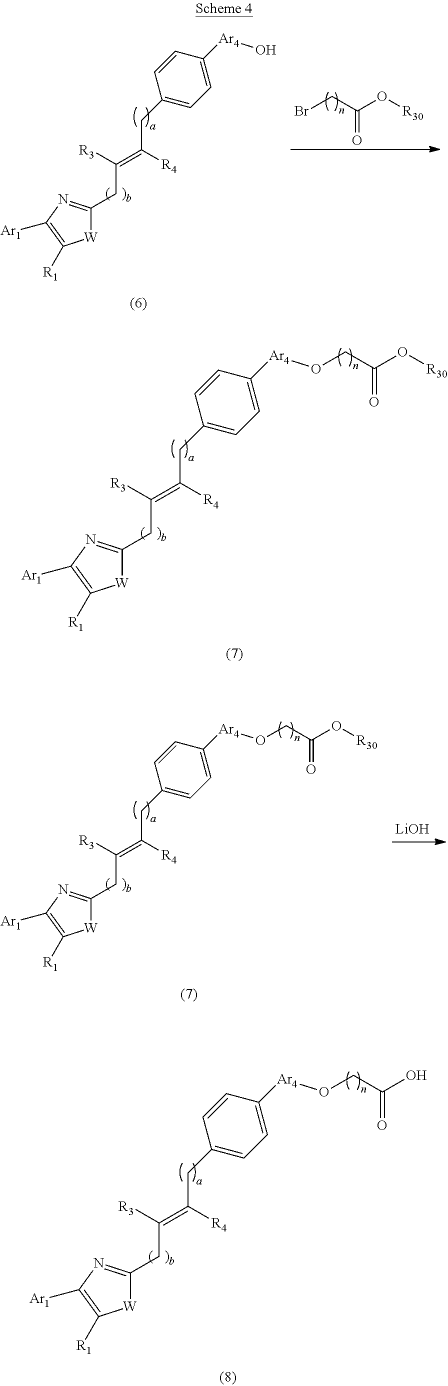

In another embodiment (Scheme 5), the imidazole nitrogen in compound (9) can be alkylated with bromo or chloro alkyl carboxylates [(Br or Cl) (CH₂)ₙCO₂R₃₀] in the presence of base such as sodium hydride, potassium tert-butoxide, or potassium carbonate using DMF, THF, or acetonitrile as the solvent at temperatures ranging from 50° C. to 100° C. Subsequent saponification of esters (10) with base such as sodium hydroxide, lithium hydroxide in aqueous and organic solvents such as THF, or methanol at temperatures ranging from room temperature to 60° C. produces carboxylic acid (11). In this instance, R₃₀ is a group such as, but not limited to, lower alkyl.

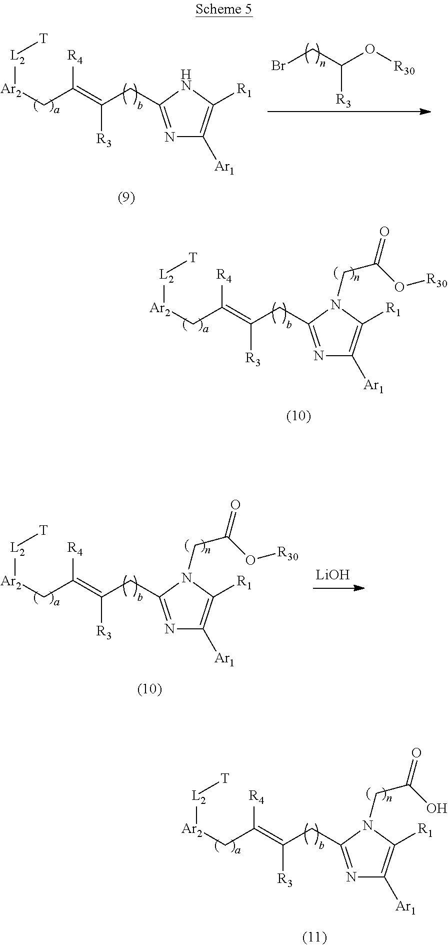

In Scheme 6 the carboxylic acids (12) can be transformed into their carboxylic acid amide analogs. This transformation can be accomplished using standard methods to effect carboxylic acid to carboxylic acid amide transformations. These methods include converting the acid to an activated acid, reacting with one or more molar equivalents of the desired amine. Methods to activate the carboxylic acid include reacting the acid with one or more molar equivalents of DIC or DIEA, with or without one or more molar equivalents of HOBt or HBTU in a suitable solvent such as dichloromethane or DMF at temperatures ranging from O° C. to 40° C. to afford amides (13). In this instance, R₃₁ is a group such as, but not limited to, -alkyl or -alkylene-aryl.

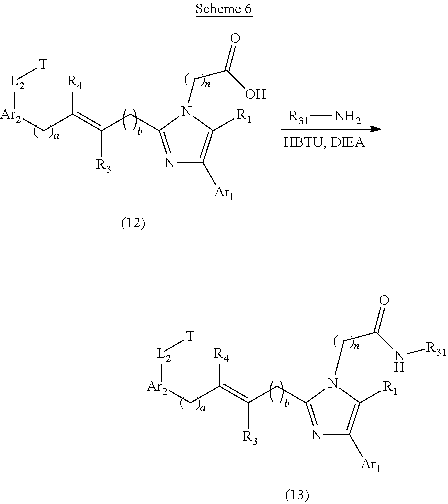

In another embodiment (Scheme 7), an imidazole nitrogen in compound (14) was alkylated with alkyl halides [(Br or Cl)(CH₂)ₙ—R₃₂] [n=1 to 6] in the presence of base such as sodium hydride, potassium tert-butoxide, or potassium carbonate using DMF, THF, or acetonitrile as the solvent at temperatures ranging from 0° C. to 80° C. afford N-alkylated products (15). In this instance R₃₂ is a group such as, but not limited to, -alkyl, aryl, or -alkenylene-aryl.

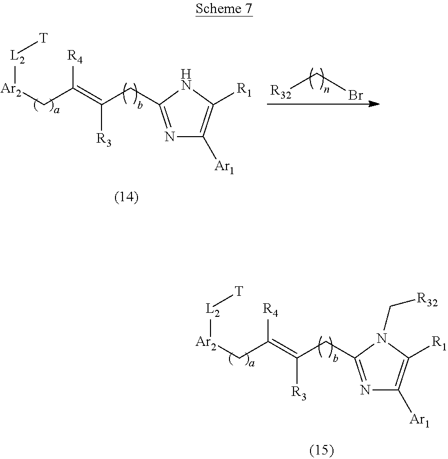

The term “amino protecting group” as used herein refers to substituents of the amino group commonly employed to block or protect the amino functionality while reacting other functional groups on the compound. Examples of such amino-protecting groups include the formyl group, the trityl group, the phthalimido group, the trichloroacetyl group, the chloroacetyl, bromoacetyl and iodoacetyl groups, urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxy-carbonyl, 2-(4-xenyl)iso-propoxycarbonyl, 1,1-diphenyleth-1-yloxycarbonyl, 1,1-diphenylprop-1-yloxycarbonyl, 2-phenylprop-2-yloxycarbonyl, 2-(p-toluoyl)prop-2-yloxycarbonyl, cyclopentanyloxycarbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluoylsulfonyl)ethoxycarbonyl, 2(methylsulfonyl)ethoxycarbonyl, 2-(triphenylphosphino)ethoxycarbonyl, 9-fluorenylmethoxycarbonyl (“FMOC”), t-butoxycarbonyl (“BOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonyl and the like; the benzoylmethylsulfonyl group, the 2-(nitro)phenylsulfenyl group, the diphenylphosphine oxide group and like amino-protecting groups. The species of amino-protecting group employed is not critical so long as the derivatized amino group is stable to the condition of subsequent reaction(s) on other positions of the compound of Formula (I) and can be removed at the desired point without disrupting the remainder of the molecule. Preferred amino-protecting groups are the allyloxycarbonyl, the t-butoxycarbonyl, 9-fluorenylmethoxycarbonyl, and the trityl groups. Similar amino-protecting groups used in the cephalosporin, penicillin and peptide art are also embraced by the above terms. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected amino” or “protected amino group” defines an amino group substituted with an amino-protecting group discussed above.

The term “hydroxyl protecting group” as used herein refers to substituents of the alcohol group commonly employed to block or protect the alcohol functionality while reacting other functional groups on the compound. Examples of such alcohol -protecting groups include the 2-tetrahydropyranyl group, 2-ethoxyethyl group, the trityl group, the trichloroacetyl group, urethane-type blocking groups such as benzyloxycarbonyl, and the trialkylsilyl group, examples of such being trimethylsilyl, tert-butyldimethylsilyl, phenyldimethylsilyl, triiospropylsilyl and thexyldimethylsilyl. The choice of alcohol-protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected hydroxyl” or “protected alcohol” defines a hydroxyl group substituted with a hydroxyl-protecting group as discussed above.

The term “carboxyl protecting group” as used herein refers to substituents of the carboxyl group commonly employed to block or protect the —OH functionality while reacting other functional groups on the compound. Examples of such alcohol -protecting groups include the 2-tetrahydropyranyl group, 2-ethoxyethyl group, the trityl group, the allyl group, the trimethylsilylethoxymethyl group, the 2,2,2-trichloroethyl group, the benzyl group, and the trialkylsilyl group, examples of such being trimethylsilyl, tert-butyldimethylsilyl, phenyldimethylsilyl, triiospropylsilyl and thexyldimethylsilyl. The choice of carboxyl protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected carboxyl” defines a carboxyl group substituted with a carboxyl-protecting group as discussed above.

Embodiments of the present invention demonstrate utility in inhibiting protein tyrosine phosphatase PTP 1B. The compounds of the present invention set forth in the present examples were found to inhibit protein tyrosine phosphatase PTP1B with inhibitory potencies (IC50's) of less than about 20 μM.

In general, embodiments of the present invention useful for pharmaceutical applications may have inhibitory potencies (IC50's) for a protein of interest of below about 100 μM. In an embodiment, embodiments of the present invention useful for pharmaceutical applications may have inhibitory potencies (IC50's) for a protein of interest of below about 50 μM. For particular applications, lower inhibitory potencies are useful. Thus, in another embodiment, compounds of the present invention may inhibit protein tyrosine phosphatase PTP1B with inhibitory potencies (IC50's) in a range of about 0.001 μM to about 10 μM. In another embodiment, compounds of the present invention may inhibit protein tyrosine phosphatase PTP1B with inhibitory potencies (IC50's) of about 0.001 μM to about 3 μM.

Embodiments of the compounds of the present invention demonstrate utility as inhibitors of protein tyrosine phosphatases (PTPases). Embodiments of the invention described herein are additionally directed to pharmaceutical compositions and methods of inhibiting PTPase activity in a mammal, which methods comprise administering, to a mammal in need of inhibition of PTPase activity, a therapeutically defined amount of a compound of Formula (I), defined above, as a single or polymorphic crystalline form or forms, an amorphous form, a single enantiomer, a racemic mixture, a single stereoisomer, a mixture of stereoisomers, a single diastereoisomer, a mixture of diastereoisomers, a solvate, a pharmaceutically acceptable salt, a solvate, a prodrug, a biohydrolyzable ester, or a biohydrolyzable amide thereof.

Thus, the present invention provides a method of inhibiting a PTPase, comprising the step of administering to a mammal in need thereof a pharmacologically effective amount of a compound of the present invention. The invention further provides a pharmaceutical composition comprising a pharmaceutically acceptable carrier and a pharmacologically effective amount of a compound of the present invention sufficient to inhibit a PTPase. A PTPase-inhibiting amount can be an amount that reduces or inhibits a PTPase activity in the subject. The compound of formula (I) may comprise a single or polymorphic crystalline form or forms, an amorphous form, a single enantiomer, a racemic mixture, a single stereoisomer, a mixture of stereoisomers, a single diastereoisomer, a mixture of diastereoisomers, a solvate, a pharmaceutically acceptable salt, a solvate, a prodrug, a biohydrolyzable ester, or a biohydrolyzable amide thereof.

Additionally provided is a pharmaceutical composition comprising a pharmaceutically acceptable carrier and a pharmacologically effective amount of a compound of Formula (I) of the present invention sufficient to treat type I diabetes.

Further, the present invention provides a pharmaceutical composition comprising a pharmaceutically acceptable carrier and a pharmacologically effective amount of a compound of Formula (I) of the present invention sufficient to treat type II diabetes.

Further, the present invention provides a pharmaceutical composition comprising a pharmaceutically acceptable carrier and a pharmacologically effective amount of a compound of Formula (I) of the present invention sufficient to treat immune dysfunction.

Further, the present invention provides a pharmaceutical composition comprising a pharmaceutically acceptable carrier and a pharmacologically effective amount of a compound of Formula (I) of the present invention sufficient to treat AIDS.

Further, the present invention provides a pharmaceutical composition comprising a pharmaceutically acceptable carrier and a pharmacologically effective amount of a compound of Formula (I) of the present invention sufficient to treat an autoimmune disease.

Further, the present invention provides a pharmaceutical composition comprising a pharmaceutically acceptable carrier and a pharmacologically effective amount of a compound of Formula (I) of the present invention sufficient to treat glucose intolerance.

Further, the present invention provides a pharmaceutical composition comprising a pharmaceutically acceptable carrier and a pharmacologically effective amount of a compound of Formula (I) of the present invention sufficient to treat obesity.

Further, the present invention provides a pharmaceutical composition comprising a pharmaceutically acceptable carrier and a pharmacologically effective amount of a compound of Formula (I) of the present invention sufficient to treat cancer.

Further, the present invention provides a pharmaceutical composition comprising a pharmaceutically acceptable carrier and a pharmacologically effective amount of a compound of Formula (I) of the present invention sufficient to treat psoriasis.

Further, the present invention provides a pharmaceutical composition comprising a pharmaceutically acceptable carrier and a pharmacologically effective amount of a compound of Formula (I) of the present invention sufficient to treat an allergic disease.

Further, the present invention provides a pharmaceutical composition comprising a pharmaceutically acceptable carrier and a pharmacologically effective amount of a compound of Formula (I) of the present invention sufficient to treat an infectious disease.

Further, the present invention provides a pharmaceutical composition comprising a pharmaceutically acceptable carrier and a pharmacologically effective amount of a compound of Formula (I) of the present invention sufficient to treat an inflammatory disease.

Further, the present invention provides a pharmaceutical composition comprising a pharmaceutically acceptable carrier and a pharmacologically effective amount of a compound of Formula (I) of the present invention sufficient to treat a disease involving the modulated synthesis of growth hormone.

Further, the present invention provides a pharmaceutical composition comprising a pharmaceutically acceptable carrier and a pharmacologically effective amount of a compound of Formula (I) of the present invention sufficient to treat a disease that involves at least in part the modulated synthesis of growth factors or cytokines that affect the production of growth hormone.

Further, the present invention provides a pharmaceutical composition comprising a pharmaceutically acceptable carrier and a pharmacologically effective amount of a compound of Formula (I) of the present invention sufficient to treat Alzheimer's disease.

The compounds of the present invention can be administered to subjects in need of inhibition of PTPase activity. Such subjects can include, for example, horses, cows, sheep, pigs, mice, dogs, cats, primates such as chimpanzees, gorillas, rhesus monkeys, and, humans. In an embodiment, a subject is a human in need of inhibition of PTPase activity.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile indictable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Also provided by the present invention are prodrugs of the invention. Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19.

Other salts which are not pharmaceutically acceptable may be useful in the preparation of compounds of the invention and these form a further aspect of the invention.

In addition, some of the compounds of the present invention may form solvates with water or common organic solvents. Such solvates are also encompassed within the scope of the invention.

Thus, in a further embodiment, there is provided a pharmaceutical composition comprising a compound of the present invention, or a pharmaceutically acceptable salt, solvate, or prodrug thereof, and one or more pharmaceutically acceptable carriers, excipients, or diluents.

The compounds of the present invention selectively act as inhibitors of one PTPase in preference to one or more other PTPases, and therefore may posess advantage in the treatment of one or more PTPase—mediated disease in preference to others.

In a further aspect, the present invention provides a method comprising administering to a human a compound of Formula I. In one embodiment, the present invention comprises method for the inhibition of PTPases. Thus, an embodiment of the present invention provides a method for treating a disease state mediated at least in part by a PTPase enzyme, comprising administering to a subject in need thereof a compound of the present invention. In alternate embodiments, the disease treated using a method of the present invention comprises acute and/or chronic inflammation, Type I diabetes, Type II diabetes, immune dysfunction, AIDS, autoimmune disease, glucose intolerance, cancer, Alzheimer's disease, psoriasis, allergic disease, graft versus host disease, infectious disease, a disease involving the modulated systhesis of growth hormone, or a disease involving at least in part the modulated synthesis of growth factors and/or cytokines that affect the production of growth hormone. In an embodiment, a pharmacologically effective amount may be administered. In another embodiment a therapeutically effective amount may be administered. In another embodiment, at least one compound of Formula (I) is utilized, either alone or in combination with one or more known therapeutic agents. In a further embodiment, the present invention provides method of prevention and/or treatment of PTPase—mediated human diseases, treatment comprising alleviation of one or more symptoms resulting from that disorder, to an outright cure for that particular disorder or prevention of the onset of the disorder, the method comprising administration to a human in need thereof a therapeutically effective amount of a compound of Formula (I).

In this method, factors which may influence what constitutes an effective amount include, but are not limited to, the size and weight of the subject, the biodegradability of the therapeutic agent, the activity of the therapeutic agent, as well as its bioavailability. As used herein, the phrase “a subject in need thereof” includes mammalian subjects, such as humans, who either suffer from one or more of the aforesaid diseases or disease states or are at risk for such. Accordingly, in the context of the therapeutic method of the invention, this method also is comprised of a method for treating a mammalian subject prophylactically, or prior to the onset of diagnosis such disease(s) or disease state(s).

The following is a non-exhaustive listing of adjuvants and additional therapeutic agents which may be utilized in combination with the PTPase inhibitors of the present invention:

Pharmacologic classifications of anticancer agents:
1. Alkylating agents: Cyclophosphamide, nitrosoureas, carboplatin, cisplatin, procarbazine
2. Antibiotics: Bleomycin, Daunorubicin, Doxorubicin
3. Antimetabolites: Methotrexate, Cytarabine, Fluorouracil
4. Plant alkaloids: Vinblastine, Vincristine, Etoposide, Paclitaxel,
5. Hormones: Tamoxifen, Octreotide acetate, Finasteride, Flutamide
6. Biologic response modifiers: Interferons, Interleukins

Pharmacologic classifications of treatment for Rheumatoid Arthritis (Inflammation)
1. Analgesics: Aspirin
2. NSAIDs (Nonsteroidal anti-inflammatory drugs): Ibuprofen, Naproxen, Diclofenac
3. DMARDs (Disease-Modifying Antirheumatic drugs): Methotrexate, gold preparations, hydroxychloroquine, sulfasalazine
4. Biologic Response Modifiers, DMARDs: Etanercept, Infliximab Glucocorticoids

Pharmacologic classifications of treatment for Diabetes Mellitus
1. Sulfonylureas: Tolbutamide, Tolazamide, Glyburide, Glipizide
2. Biguanides: Metformin
3. Miscellaneous oral agents: Acarbose, PPAR agonists such as Troglitazone, DPP-IV inhibitors, Glucokinase activators
4. Insulin, insulin mimetics, insulin secretagogues, insulin sensitizers
5. GLP-1, GLP-1 mimetics

Pharmacologic classifications of treatment for Alzheimer's Disease
1. Cholinesterase Inhibitor: Tacrine, Donepezil
2. Antipsychotics: Haloperidol, Thioridazine
3. Antidepressants: Desipramine, Fluoxetine, Trazodone, Paroxetine
4. Anticonvulsants: Carbamazepine, Valproic acid

Pharmacologic classifications of treatment for Hyperlipidemia
1. HMG CoA reductase inhibitors Inhibitor: Mevinolin
2. cholestyramine
3. fibrates

In a further embodiment, the present invention provides a method of treating diseases mediated at least in part by a PTPase enzyment (iPTPase mediated diseases), the method comprising administering to a subject in need thereof, a therapeutically effective amount of a compound of Formula (I) in combination with a therapeutic agent. Examples of combination therapeutic agents may include, but are not limited to, alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, or fibrates.

Generally speaking, a compound of Formula (I) may be administered at a dosage level of from about 0.003 to 500 mg/kg of the body weight of the subject being treated. In an embodiment, a compound of Formula (I) may be administered at a dosage range between about 0.003 and 200 mg/kg of body weight per day. In an embodiment, a compound of Formula (I) may be administered at a dosage range between about 0.1 to 100 mg/kg of body weight per day. The amount of active ingredient that may be combined with the carrier materials to produce a single dosage may vary depending upon the host treated and the particular mode of administration. For example, a formulation intended for oral administration to humans may contain 1 mg to 2 grams of a compound of Formula (I) with an appropriate and convenient amount of carrier material which may vary from about 5 to 95 percent of the total composition. Dosage unit forms may generally contain between from about 5 mg to about 500 mg of active ingredient. This dosage may be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

### EXAMPLES

The general procedures used in the methods of the present invention are described below.

### General Experimental

LC-MS data was obtained using gradient elution on a Waters 600 controller equipped with a 2487 dual wavelength detector and a Leap Technologies HTS PAL Autosampler using an YMC Combiscreen ODS-A 50×4.6 mm column. A three minute gradient was run from 25% B (97.5% acetonitrile, 2.5% water, 0.05% TFA) and 75% A (97.5% water, 2.5% acetonitrile, 0.05% TFA) to 100% B. The mass spectrometer used was a Micromass ZMD instrument. All data was obtained in the positive mode unless otherwise noted. ¹H NMR data was obtained on a Varian 400 MHz spectrometer.

### General Procedure A: Imidazole Formation

To a mixture of a carboxylic acid (1 eq) and an aromatic acyl bromide (2 eq) in anhydrous DMF (0.1-0.5 M) was added DIEA (3 eq). The reaction mixture was stirred at room temperature under nitrogen for 6 to 8 hours. After that, it was poured into water, acidified with 10% citric acid and extracted with ethyl acetate. The organic extract was washed with water and brine, dried over Na₂SO₄. After evaporation of the solvent, the pale-brown residue was recrystallized from EtOAc-Hexanes, dried and used directly in the next step.

The intermediate obtained above was dissolved in glacial acetic acid (0.1-0.5 M), and ammonium acetate (20 eq) was added. The mixture was then heated at 120° C. under nitrogen for 8 to 10 hours. At completion, it was poured into water, neutralized with saturated sodium bicarbonate and extracted with ethyl acetate. The organic extract was washed with water and brine, and dried over Na₂SO₄. After removal of the solvent in vacuo, the residue was purified by flash column chromatography to afford the desired product.

### General Procedure B: Boronic Acid Coupling

To a solution of the bromo compound (1 eq) in a 2:1 mixture of toluene and ethanol (0.1-0.5 M) was added the appropriate boronic acid (1.2 eq) and a catalytic amount of tetrakis(triphenylphosphine)palladium(0) (0.05 eq), followed by 2 M sodium carbonate solution in water (30 eq). The reaction mixture was stirred at 90° C. under nitrogen for 6 hours. After cooling, the reaction mixture was diluted with water and extracted with ethyl acetate. The organic extract was washed with water and brine, and dried over Na₂SO₄. After removal of the solvent in vacuo, the residue was purified by flash column chromatography to afford the desired compound.

### General Procedure C: Dealkylation

To the solution of alkyl phenolic ether (1 eq) in anhydrous DCM (0.1-0.5 M) at −20° C. was added dropwise BBr₃ (2 eq, solution in anhydrous DCM). The solution was warmed to room temperature over 30 minutes, and the reaction mixture quenched with ice water. The reaction mixture was then diluted with water/EtOAc and the layers were separated. The aqueous layer was further extracted with EtOAc, and the organic layers combined, washed with water and brine, and dried over Na₂SO₄. The solvent was removed in vacuo, and the residue subjected to silica gel chromatography to yield the final product.

### General Procedure D: Hydrogenation of Double Bond

To 1 equivalent of the desired alkene suspension in ethyl acetate (0.1-0.5 M) was added a catalytic amount of platinum(IV) oxide (wet). After degassing and introducing of nitrogen and degassing again, hydrogen was introduced through a hydrogen balloon. The reaction mixture was stirred at room temperature for 0.5 hour. The reaction mixture was then filtered through celite, the celite cake was washed three times with ethyl acetate, and the filtrates combined. The solvent was then removed in vacuo, and the residue was purified by silica gel chromatography to afford the desired compound.

### General Procedure E: Alkylation of Imidazole Nitrogen or Phenolic Oxygen

To a solution of imidazole or phenol (1 eq) in anhydrous DMF (0.1-0.5 M) was added an alkyl or aryl halide (2 eq) followed by freshly ground K₂CO₃ (4 eq). The reaction mixture was heated at 100° C. under nitrogen for 2 hours. The mixture was then diluted with water/EtOAc and the layers separated. The aqueous layer was further extracted with EtOAc, and the organic layers combined and dried over Na₂SO₄. The solvent was removed in vacuo and the residue was purified by silica gel chromatography to yield the final product.

### General Procedure F: Hydrolysis of Ester

The ester (1 eq) was suspended in a mixture of MeOH:THF:H₂O (1:1:1 ; 0.1-0.2 M). LiOH (10-15 eq) was added and the mixture stirred at 40° C. for 3 hours. The solution was acidified with 10% citric acid solution, and extracted with ethyl acetate. The organic extracts were combined, washed with brine, dried over Na₂SO₄, and the solvent removed in vacuo. The residue was purified by silica gel chromatography to yield the final compound.

### General Procedure G: Coupling of Carboxylic Acid and Amine

To a solution of carboxylic acid (1.1 eq) in DMF (0.1-0.5 M), HBTU (1.1 eq) was added followed by DIEA (1.2 eq) and the appropriate protected amine (1 eq.). The reaction mixture was then stirred at room temperature for 4 hours. At completion, the reaction mixture was diluted with water/EtOAc, acidified with 10% citric acid, and the layers were separated. The combined organic layer was washed with water, saturated NaHCO₃ and brine, dried over Na₂SO₄ and filtered. The filtrate was concentrated and purified by silica gel chromatography to afford the amide derivative.

### General Procedure H: Sonogashira Coupling

To a solution of aryl bromide or aryl iodide (1 eq) in anhydrous DMF (0.1-0.5 M) was added the appropriate terminal acetylene (1.2 eq) followed by tetrakis (triphenylphosphine)palladium(0) (0.05 eq), CuI (0.1 eq), and DIEA (2 eq). The reaction mixture was then heated at 120° C. under nitrogen for 6-8 hours. At completion, the reaction mixture was diluted with water/EtOAc, acidified with 10% citric acid, and the layers separated. The combined organic layers was washed with water and brine, dried over Na₂SO₄ and filtered. The filtrate was concentrated and purified by silica gel chromatography to afford the acetylene derivative.

### General Procedure I: Diaryl Ether Formation Using Aryl Fluoride

To a solution of phenol compound (1 eq) in anhydrous DMF (0.1-0.5 M), the appropriate activated aryl fluoride (1.5 eq) was added followed by Cs₂CO₃ (3 eq). The reaction mixture was then heated at 120° C. under nitrogen for 2 hours. At completion, the reaction mixture was diluted with water/EtOAc and the layers separated. The aqueous layer was reextracted with EtOAc and the organic layers combined, washed with water and brine. The organic phase was then dried over Na₂SO₄, filtered, and the filtrate was concentrated and purified by silica gel chromatography to afford the diaryl ether derivative.

### General Procedure J: Ullmann Diaryl Ether Coupling

To a solution of phenol compound (1 eq) in anhydrous NMP (0.1-0.5 M), the appropriate aryl bromide or iodide (1.5 eq) was added followed by CuCl (0.2 eq), 2,2,6,6-tetramethyl-3,5-heptanedione (0.2 eq) and Cs₂CO₃ (3 eq). The reaction mixture was then heated at 120° C. under nitrogen for 6 to 8 hours. At completion, the reaction mixture was diluted with water/EtOAc and the layers separated. The aqueous layer was reextracted with EtOAc and the organic layers combined, washed with water and brine. The organic phase was then dried over Na₂SO₄, filtered, and the filtrate was concentrated and purified by silica gel chromatography to afford the diaryl ether derivative.

### General Procedure K: Reduction of Aryl Nitro Group

To a suspension of aryl nitro compound (1 eq) in HOAc (0.1-0.5 M), iron powder (−325 mesh, 4 eq) was added and the mixture was then heated at 120° C. under nitrogen for 3 to 4 hours. At completion, the reaction mixture was diluted with water/EtOAc and the leftover iron powder was filtered and washed with EtOAc. The combined organic layer was washed with water, saturated NaHCO₃ and brine. The organic phase was then dried over Na₂SO₄, filtered, and the filtrate was concentrated and purified by silica gel chromatography to afford the aniline derivative.

General Procedure L: Coupling of Aniline with Sulfonyl Chloride or Sulfonic Anhydride

To a suspension of aniline compound (1 eq) in anhydrous DCM (0.1-0.5 M) at 0° C. was added DIEA (1.2 eq) followed by the appropriate sulfonyl chloride or sulfonic anhydride (1.1 eq, diluted in anhydrous DCM). The reaction mixture was then warmed up and stirred at room temperature under nitrogen for 3 to 4 hours. At completion, the reaction mixture was diluted with water/EtOAc and the layers separated. The aqueous layer was reextracted with EtOAc and the organic layers combined, washed with 10% citric acid, water and brine. The organic phase was then dried over Na₂SO₄, filtered, and the filtrate was concentrated and purified by silica gel chromatography to afford the sulfonamide derivative.

### General Procedure M: Formation of Tetrazole

To a solution of phenol compound (1 eq) in anhydrous DMF (0.1-0.5 M) was added an appropriate bromoalkylnitrile (2 eq) followed by freshly ground K₂CO₃ (4 eq). The reaction mixture was heated at 100° C. under nitrogen for 2 hours. The mixture was then diluted with water/EtOAc and the layers separated. The aqueous layer was further extracted with EtOAc, and the organic layers combined and dried over Na₂SO₄. The solvent was removed in vacuo and the residue purified by silica gel chromatography to yield the nitrile intermediate.

The nitrile intermediate (1 eq) obtained above was dissolved in anhydrous DMF (0.1-0.5 M) and sodium azide (10 eq) and ammonium chloride (10 eq) were added. The reaction mixture was heated at 120° C. under nitrogen for 8 to 10 hours. At completion, the reaction mixture was diluted with water/EtOAc and the layers separated. The aqueous layer was further extracted with EtOAc, and the organic layers combined and dried over Na₂SO₄. The solvent was removed in vacuo and the residue was purified by silica gel chromatography to afford the final product.

General Procedure N: Protection of imidazole nitrogen 1 equivalent of an imidazole was suspended in anhydrous THF (0.1-0.5 M), to which was added 1.4 equivalents of TEA and 1.5 equivalents of di-tert-butyl-dicarbonate. The mixture was stirred for 2 hours and diluted with water and the layers were separated. The aqueous layer was further extracted with EtOAc, the organic layers combined, washed with brine, and the organic layer dried over sodium sulfate. The solvent was removed in vacuo, and the crude product purified by flash chromatography on silica gel to give the final product.
General Procedure O: Removal of the t-Butyl Carbamate Group

The protected compound was stirred in 4N HCl/dioxane for 1 hour. The solvent removed, and the product triturated several times with ether to afford the desired compound.

### General Procedure P: Alkylation.

To a solution of imidazole or phenol (1 eq) in anhydrous DMF (0.1-0.5M) was added 1-2 eq sodium hydride, either solid or as a suspension in DMF or THF. The mixture was stirred at room temperature for 20 min and a solution of alkyl or aryl halide (1-3 eq) was added in DMF or THF. Stirring continued for 1 hour, then the mixture was diluted with water/EtOAc and neutralized with 10% aqueous citric acid. The organic layer was washed with brine, dried over Na₂SO₄, and evaporated in vacuo. The residue was purified by silica gel chromatography to provide the final product.

### General Procedure Q: Benzimidazole Formation

To a solution of an aldehyde (1 eq) in ethanol (0.1-0.5 M) was added 1.5 eq of a benzenediamine. The mixture was sealed in a heavy walled glass tube with stir bar and stirred at 100° C. for 2 hours to overnight. The mixture was then evaporated and taken up in water/EtOAc and layers were separated. The aqueous layer was further extracted with EtOAc and the combined organic extracts were washed with brine, dried over Na₂SO₄, and evaporated in vacuo. The residue was purified by silica gel chromatography to give the product.

General Procedure R: Catalytic reduction of aryl nitro group

To a solution of aryl nitro compound (1 eq) in methanol (0.1-0.5 M) was added 0.1 eq of 10% Pd/C catalyst. The flask was flushed with H₂ and stirred under H₂ pressure (balloon) overnight at room temperature. The mixture was then filtered on a celite pad and evaporated, and the residue was purified by silica gel column chromatography to provide the desired product.

### General Procedure S: Silyl Group Deprotection

To a solution of O- or N-silyl compound (1 eq) in THF (0.1-0.5 M) was added 5 eq of tetrabutylammonium fluoride as a solution in THF. The mixture was stirred at 65° C. for 1-3 hours, then was evaporated to a small volume and taken up in water/EtOAc. Layers were separated and the aqueous layer was further extracted with EtOAc. The combined organic extracts were washed with brine, dried over Na₂SO₄, and evaporated in vacuo. The residue was purified by silica gel column chromatography to give the desired product.

### General Procedure T: Selective Trimethylsilyl Group Deprotection

To a solution of trimethylsilyl compound (1 eq) in anhydrous methanol (0.1-0.5 M) was added 10 eq anhydrous K₂CO₃ under nitrogen. The mixture was stirred under nitrogen at room temperature for 3 hours, then diluted with water/EtOAc and layers were separated. The aqueous layer was further extracted with EtOAc and the combined organic layers were washed with brine, dried over Na₂SO₄ and evaporated in vacuo. The residue was purified by silica gel column chromatography to provide the desired product.

### General Procedure U: Reductive Amination

To a solution of amine (1 eq) in 1,2-dichloroethane (0.1-0.5 M) was added an aldehyde (1.2 eq) and a catalytic amount of acetic acid. The mixture was stirred at room temperature for 30 minutes under nitrogen, then sodium triacetoxyborohydride (3 eq) was added and the mixture was allowed to stir for 12-16 hours at room temperature. The mixture was then diluted with water/EtOAc and layers were separated. The aqueous layer was extracted additionally with EtOAc and the combined organic extracts were washed with water, brine, dried over Na₂SO₄ and evaporated in vacuo. The residue was purified by silica gel column chromatography to provide the desired product.

### General Procedure V: Saturation of Double Bond

To a suspension of double bond containing compound (1 eq) in HOAc (0.1-0.5 M) was added iron powder (−325 mesh, 10-20 eq) and the mixture was stirred and heated at 120° C. for 18-24 hours. The mixture was then diluted with water/EtOAc and filtered to remove excess iron powder, then layers were separated and the aqueous layer was washed again with EtOAc. The combined organic extracts were washed with water, saturated NaHCO₃, and brine, then dried over Na₂SO₄. After evaporation in vacuo, the residue was purified by silica gel column chromatography to provide the desired product.

### General Procedure W: Evans Coupling

To a solution of phenol compound (1 eq) in anhydrous DCM (0.1-0.5 M) was added Cu(OAc)₂ (1 eq), arylboronic acid (3 eq), and powdered 4 A molecular sieves, followed by DIEA (5 eq). The colored heterogeneous reaction mixture was then stirred at room temperature under ambient atmosphere for 18 hours to 2 days. At completion, the resulting slurry was filtered through celite, washed with DCM. The combined organic layers was washed with water and brine, dried over Na₂SO₄ and filtered. The filtrate was concentrated and purified by silica gel chromatography to afford the diaryl ether derivative.

### General Procedure X: Oxidation of Benzylic Methylene Carbon

To a solution of benzylic compound (1 eq) in acetic acid (0.1-0.5 M) was added selenium dioxide (10 eq). The colored heterogeneous reaction mixture was then stirred at reflux under ambient atmosphere for 2 to 3 days. At completion, the resulting slurry was filtered through celite, washed with ethyl acetate. The combined organic layers was washed with water and brine, dried over Na₂SO₄ and filtered. The filtrate was concentrated and purified by silica gel chromatography to afford the keto derivative.

### General Procedure Y: Preparation of Sulfahydantoin Derivatives

### Procedure Y1: Reduction of Aryl Nitro Group

To a suspension of aryl nitro compound (1 eq) in HOAc (0.1-0.5 M), iron powder (−325 mesh, 8 eq) was added and the mixture was then heated at 80° C. under nitrogen for 5-10 minutes. The reaction mixture was then diluted with water/EtOAc and the leftover iron powder was filtered and washed with EtOAc. The combined organic layer was washed with water, saturated NaHCO₃ and brine. The organic layer was then dried over Na₂SO₄, filtered, and the filtrate was concentrated and purified by silica gel chromatography to afford the aniline derivative.

### Procedure Y2: Alkylation of Aniline

To a suspension of aniline compound (1 eq) in anhydrous DMF (0.1-0.5 M) at room temperature was added 1.2 eq of α-bromo ester followed by 2.5 eq of DIEA. The reaction mixture was then stirred at room temperature under nitrogen for 18 hours. Alternately, to a suspension of aniline compound (1 eq) in anhydrous DMF (0.1-0.5 M) at room temperature was added 2 eq of α-bromo ester followed by 5 eq of anhydrous cesium carbonate. The reaction mixture was then stirred at 120° C. under nitrogen for 18 hours. The reaction mixture was then diluted with water/EtOAc and the layers separated. The aqueous layer was repeatedly extracted with EtOAc and the organic layers were combined and washed with water and brine. The organic phase was then dried over Na₂SO₄, filtered, and the filtrate was concentrated and purified by silica gel chromatography to afford the α-anilino ester derivative.

### Procedure Y3: Formation of Sulfahydantoin

Step 1: To a solution of chlorosulfonyl isocyanate (1.5 eq) in anhydrous 1,2-dichloroethane (0.1-0.5 M) at 0° C. was added 1.5 eq of tert-butanol as a solution in anhydrous 1,2-dichloroethane (0.5 M). The mixture was allowed to warm to room temperature while stirring and was then cooled to 0° C. again. A suspension of anilino ester from general procedure Y2 (1.0 eq) in 1,2-dichloroethane (0.3-0.5 M) and 2.5 eq DIEA was cooled to 0° C. and the chlorosulfonyl isocyanate-tert-butanol mixture was added dropwise while stirring. The mixture was stirred at room temperature for 1 hour, then diluted with water/CH₂Cl₂ and the layers separated. The organic layers were combined and washed with water and brine. The organic phase was then dried over Na₂SO₄, filtered, and the filtrate was concentrated and purified by silica gel chromatography to afford the aniline N-Boc sulfonylurea derivative.

Step 2: Boc protected compound was stirred in dichloromethane/trifluoroacetic acid for 30 minutes. The solvent was removed and the residue was triturated several times with ether to afford the deprotected compound.

Step 3: To a suspension of the deprotected aniline N-sulfonyl compound in ethanol (0.1-0.5 M) was added 5.0 eq of NaOH as a 2 M solution in water. The mixture was stirred at room temperature for 5-7 minutes, then diluted with 2% citric acid/EtOAc and the layers separated. The organic layer was washed with water and brine. The organic layer was then dried over Na₂SO₄, filtered, and the filtrate was concentrated and purified by silica gel chromatography to afford the sulfahydantoin product.

### General Procedure Z: Alkylation of Sulfahydantoin

To a suspension of sulfahydantoin compound (1 eq) in anhydrous DMF (0.1-0.5 M) at 0° C. was added 1.5 eq of NaH followed by 1.5 eq of MeI. The reaction mixture was then stirred at 0° C. under nitrogen for 0.5 hours. At completion, it was diluted with water/EtOAc and the layers separated. The aqueous layer was reextracted with EtOAc and the organic layers were combined and washed with water and brine. The organic phase was then dried over Na₂SO₄, filtered, and the filtrate was concentrated and purified by silica gel chromatography to afford the N-methylated sulfahydantoin derivative.

### General Procedure AA: Preparation of N-acyl-alkanesulfonamids

To a solution of acid (1 eq) in anhydrous THF (0.1-0.5 M) was added to CDI (3 eq). The reaction mixture was stirred at room temperature for 10-12 hours for complete conversion of acid to mixed anyhydride. A mixture of DBU (1.5 eq) and appropriate sulfonamide (1.5 eq) in anhydrous THF (0.1 M) was added to the reaction mixture and was refluxed for 6-8 hours. At completion the reaction mixture was diluted with water/EtOAc and the layers were separated. The aqueous layer was extracted with EtOAc and the organic layers combined, washed with water and brine. The organic phase was dried over Na₂SO4, filtered and the filtrate was concentrated and purified by silica gel chromatography to afford the acylsulfonamide derivative.

### General Procedure AB: Preparation of C-sulfahydantoin Derivatives

To a suspension of aryl aldehyde in ethanol (0.1-0.5 M) was added sodium cyanide (20 eq) and sulfamide (10 eq). The mixture was heated at reflux under nitrogen for 18 hours. The mixture was then diluted with aqueous NaHCO₃/EtOAc and the layers were separated. The aqueous layer was washed with EtOAc and the combined organic layers were washed with water and brine, then dried over Na₂SO₄ and concentrated. The residue was purified by silica gel column chromatography to afford the iminosulfahydantoin derivative.

To a suspension of iminosulfahydantoin in ethanol (0.1-0.5 M) was added concentrated HCl (100 eq). The mixture was heated at reflux 12-18 hours, then diluted with aqueous NaHCO₃/EtOAc and the layers were separated. The aqueous layer was washed with EtOAc and the combined organic layers were washed with water and brine, then dried over Na₂SO₄ and concentrated. The residue was purified by silica gel column chromatography to afford the α-N-sulfonamide ethyl ester.

To a suspension of α-N-sulfonamide ethyl ester in dry methanol (0.1-0.5 M) was added sodium methoxide (5 eq) in methanol. The mixture stirred 15 min under nitrogen at room temperature. The mixture was then diluted with 2% citric acid and EtOAc and the layers were separated. The aqueous layer was washed with EtOAc and the combined organic layers were washed with water and brine, then dried over Na₂SO₄ and concentrated. The residue was purified by silica gel column chromatography to afford the sulfahydantoin product.

### Example 1

4-(2,4-Dichloro-phenyl)-2-[2-(4-methoxy-phenyl)-(E)-vinyl]-1H-imidazole (3.45 g, 10 mmol) was treated with methyl bromoacetate as described in general procedure E followed by ester hydrolysis as described in general procedure F to afford {4-(2,4-dichloro-phenyl)-2-[2-(4-methoxy-phenyl)-(E)-vinyl]-imidazol-1-yl}-acetic acid (2.26 g, 56% yield).

LCMS: m/z 403 (M+H)⁺; ¹H NMR (CD₃OD, 400 MHz): δ 3.82 (s, 3H), 4.97 (s, 2H), 6.88 (d, 1H), 6.95 (d, 2H), 7.33 (d, 1H), 7.51 (d, 2H), 7.52 (d, 1H), 7.54 (s, 1H), 7.66 (d, 1H), 7.93 (s, 1H) ppm.

### Example 2

4-(2,4-Dichloro-phenyl)-2-fluoren-9-ylidenemethyl-1H-imidazole (389 mg, 1 mmol) was treated with methyl bromoacetate as described in general procedure E followed by ester hydrolysis as described in general procedure F to afford [4-(2,4-dichloro-phenyl)-2-fluoren-9-ylidenemethyl-imidazol-1-yl]-acetic acid (260 mg, 58% yield).

LCMS: m/z 447 (M+H)⁺; ¹H NMR (CD₃OD, 400 MHz): δ 5.02 (s, 2H), 7.25 (m, 1H), 7.37-7.51 (m, 5H), 7.57 (dd, 1H), 7.73 (d, 1H), 7.77-7.82 (m, 3H), 7.93 (d, 1H), 8.08 (s, 1H) ppm.

### Example 3

4-(2,4-Dichloro-phenyl)-2-fluoren-9-ylidenemethyl-1H-imidazole (39 mg, 0.1 mmol) was treated with methyl 1-bromobutyrate as described in general procedure E followed by ester hydrolysis as described in general procedure F to afford 4-[4-(2,4-dichloro-phenyl)-2-fluoren-9-ylidenemethyl-imidazol-1-yl]-butyric acid (23 mg, 48% yield).

LCMS: m/z 475 (M+H)⁺; ¹H NMR (CD₃OD, 400 MHz): δ 2.14 (m, 2H), 2.40 (t, 2H), 4.32 (t, 2H), 7.26 (m, 1H), 7.33 (m, 1H), 7.39 (t, 2H), 7.44 (dd, 1H), 7.53 (s 1H), 7.56 (dd, 1H), 7.75 (t, 2H), 7.97 (s, 1H), 8.02 (d, 1H), 8.12 (d, 1H), 8.83 (d, 1H) ppm.

### Example 4

4-(2,4-Dichloro-phenyl)-2-[2-(4′-methoxy-biphenyl-4-yl)-(E)-vinyl]-1H-imidazole (421 mg, 1 mmol) was treated with methyl bromoacetate as described in general procedure E followed by ester hydrolysis as described in general procedure F to afford {4-(2,4-dichloro-phenyl)-2-[2-(4′-methoxy-biphenyl-4-yl)-(E)-vinyl]-imidazol-1-yl}-acetic acid (268 mg, 56% yield).

LCMS: m/z 479 (M+H)⁺; ¹H NMR (CD₃OD, 400 MHz): δ 3.82 (s, 3H), 4.95 (s, 2H), 7.03 (d, 2H), 7.15 (d, 1H), 7.58-7.61 (m, 3H), 7.68-7.70 (m, 6H), 7.73 (d, 1H), 7.90 (s, 1H) ppm.

### Example 5

4-(2,4-Dichloro-phenyl)-2-[2-(4′-methoxy-biphenyl-4-yl)-(E)-vinyl]-1H-imidazole (42 mg, 0.1 mmol) was demethylated as described in the general procedure C and the resulting intermediate was treated with 2 equivalents of methyl 4-bromobutyrate as described in the general procedure E followed by ester hydrolysis as described in the general procedure F to afford 4-[2-{2-[4′-(3-carboxy-propoxy)-biphenyl-4-yl]-(E)-vinyl}-4-(2,4-dichloro-phenyl)-imidazol-1-yl]-butyric acid (16 mg, 27% yield).

LCMS: m/z 579 (M+H)⁺.

### Example 6

4-(2,4-Dichloro-phenyl)-2-[2-(4′-methoxy-biphenyl-4-yl)-(E)-vinyl]-1H-imidazole (42 mg, 0.1 mmol) was treated with methyl 1-bromobutyrate as described in general procedure E followed by ester hydrolysis as described in general procedure F to provide 4-{4-(2,4-dichloro-phenyl)-2-[2-(4′-methoxy-biphenyl-4-yl)-(E)-vinyl]-imidazol-1-yl}-butyric acid (27 mg, 53% yield).

LCMS: m/z 507 (M+H)⁺.

### Example 7

{4-Biphenyl-4-yl-2-[2-(4-methoxy-phenyl)-(E)-vinyl]-imidazole-1-yl)-acetic acid methyl ester (212 mg, 0.5 mmol) was hydrolyzed according to general procedure F to give {4-biphenyl-4-yl-2-[2-(4-methoxy-phenyl)-(E)-vinyl]-imidazole-1yl}-acetic acid (212 mg, 80%).

LCMS: m/z 411 (M+H)⁺; ¹H NMR (CDCl₃, 400 MHz): δ 3.78 (s, 3H), 5.17 (s, 2H), 6.95-6.93 (m, 2H), 7.36-7.33 (m, 2H), 7.48-7.44 (m, 2H), 7.55-7.53 (m, 2H), 7.71-7.64 (m, 6H), 7.90-7.88 (m, 2H) ppm.

### Example 8

4-(4-(2,4-Dichloro-phenyl)-2-{2-[4′-(3-methoxycarbonyl-propoxy)-biphenyl-3-yl]-(E)-vinyl}-imidazol-1-yl)-butyric acid methyl ester (421 mg, 69%) was prepared according to general procedure A using trans-3-bromocinnamic acid (227 mg, 1 mmol) and 2-bromo-2,4-dichloroacetophenone (267 mg, 1 mmol) and obtained 2-[2-(3-bromo-phenyl)-(E)-vinyl]-4-(2,4-dichloro-phenyl)-1H-imidazole (394 mg, 1 mmol) was coupled with 4-hydroxyphenylboronic acid (137 mg, 1 mmol) following general procedure B and resulting 3′-{2-[4-(2,4-dichloro-phenyl)-1H-imidazol-2-yl]-(E)-vinyl}-biphenyl-4-ol (407 mg, 1 mmol) was di-alkylated with methyl 4-bromobutyrate (362 mg, 2 mmol) following general procedure E.

LCMS: m/z 607 (M+H)⁺; ¹H NMR (CDCl₃, 400 MHz): δ 2.18 (m, 2H), 2.42 (t, 3H), 2.56 (t, 3H), 3.66 (s, 3H), 3.70 (s, 3H), 4.06 (q, 2H), 4.20 (q, 2H), 6.96 (d, 2H), 7.07 (d, 2H), 7.31 (d, 1H), 7.33-7.42 (m, 2H), 7.44-7.52 (m, 2H), 7.56 (d, 2H), 7.64 (s, 1H), 7.77 (d, 1H), 8.27 (d, 1H) ppm.

### Example 9

4-(4-(2,4-Dichloro-phenyl)-2-{2-[4′-(3-methoxycarbonyl-propoxy)-biphenyl-3-yl]-(E)-vinyl}-imidazol-1-yl)-butyric acid methyl ester (304 mg, 0.5 mmol) was hydrolyzed according to general procedure F to give 4-[2-{2-[4′-(3-carboxy-propoxy)-biphenyl-3-yl]-(E)-vinyl}-4-(2,4-dichloro-phenyl)-imidazol-1-yl]-butyric acid (212 mg, 73%).

LCMS: m/z 579 (M+H)⁺; ¹H NMR (DMSO-d₆, 400 MHz): δ 1.96 (m, 2H), 2.28 (t, 3H), 2.42 (t, 3H), 4.03 (q, 2H), 4.25 (q, 2H), 7.03 (d, 2H), 7.40-7.55 (m 4H), 7.61-7.65 (m, 4H), 7.67-7.69 (m, 2H), 7.94 (d, 1H), 8.26 (d, 1H) ppm.

### Example 10

4-(3′-{2-[4-(2,4-Dichloro-phenyl)-1-methoxycarbonylmethyl-1H-imidazol-2-yl]-(E)-vinyl}-biphenyl-4-yloxy)-butyric acid methyl ester (379 mg, 65%) was prepared according to general procedure A using trans 3-bromo cinnamic acid (227 mg, 1 mmol) and 2-bromo-2,4-dichloro acetophenone (267 mg, 1 mmol) and obtained 2-[2-(3-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(3-Bromo-phenyl)-(E)-vinyl]-4-(2,4-dichloro-phenyl)-1H-imidazol-1-yl]-acetic acid methyl ester (466 mg, 1 mmol) was coupled with 4-hydroxy phenyl boronic acid (137 mg, 1 mmol) following general procedure B and resulting 4{-(2,4-dichloro-phenyl)-2-[2-(4′-hydroxy-biphenyl-3-yl]imidazol-1-yl} acetic acid methyl ester (479 mg, 1 mmol) was alkylated with 4-bromomethyl butyrate (181 mg, 1 mmol) following general procedure E.

LCMS: m/z 579 (M+H)⁺.

### Example 11

4-(3′-{2-[4-(2,4-Dichloro-phenyl)-1-methoxycarbonylmethyl-1H-imidazol-2-yl]-(E)-vinyl}-biphenyl-4-yloxy)-butyric acid methyl ester (290 mg, 0.5 mmol) was hydrolyzed according to general procedure F to give 4-(3′-{2-[4-(2,4-dichloro-phenyl)-1-methoxycarbonylmethyl-1H-imidazol-2-yl]-(E)-vinyl}-biphenyl-4-yloxyybutyric acid (382 mg, 69%).

LCMS: m/z 551 (M+H)⁺; ¹H NMR (DMSO-d₆, 400 MHz): δ 1.98 (m, 2H), 2.42 (t, 2H), 4.03 (t, 2H), 5.17 (d, 2H), 7.03 (d, 1H), 7.30 (s, 1H), 7.34 (s, 1H), 7.38-7.49 (m, 2H), 7.50-7.54 (m, 2H), 7.55-7.71 (m, 4H), 7.94 (d, 1H), 7.97 (d, 1H), 8.30 (d, 1H) ppm.

### Example 12

2-[2-(6-Benzyloxy-naphthalen-2-yl)-(E)-vinyl]-4-(2,4-dichloro-phenyl)-imidazol-1-yl]-acetic acid methyl ester (139 mg, 51%) was prepared according to general procedure A using trans-3-(6-methoxy naphthalene-2-yl)acrylic acid (Rwerechem-BKHW-0217) (228 mg, 1 mmol) and 2-bromo-2,4-dichloro acetophenone (267 mg, 1 mmol) and obtained 4-(2,4-dichloro-phenyl)-2[2-(6-methoxy-naphthalen-2-yl)-(E)-vinyl]-1H-imidazol (197 mg, 0.5 mmol) was alkylated with methyl bromo acetate (77 mg, 0.5 mmol) following general procedure E. The resulted 4-(2,4-dichloro-phenyl)-2-[2-(6-methoxy-naphthalen-2-yl)-(E)-vinyl]-imidazol-1-yl}-acetic acid methyl ester (233 mg, 0.5 mmol) was de-alkylated as described in general procedure C and obtained 4-(2,4-dichloro-phenyl)-2-[2-(6-hydroxy-naphthalen-2-yl)-(E)-vinyl]-imidazol-1-yl}-acetic acid methyl ester (227 mg, 0.5 mmol) was alkylated with benzyl bromide (171 mg, 1 mmol) following general procedure E.

LCMS: m/z 543 (M+H)⁺.

### Example 13

2-[2-(6-Benzyloxy-naphthalen-2-yl)-(E)-vinyl]-4-(2,4-dichloro-phenyl)-imidazol-1-yl]-acetic acid methyl ester (135 mg, 0.25 mmol) was hydrolyzed according to general procedure F to give 2-[2-(6-Benzyloxy-naphthalen-2-yl)-(E)-vinyl]-4-(2,4-dichloro-phenyl)-imidazol-1-yl]-acetic acid methyl ester (75 mg, 57%).

LCMS: m/z 529 (M+H)⁺; ¹H NMR (DMSO-d₆, 400 MHz): δ 5.17 (s, 2H), 5.23 (s, 2H), 7.15 (d, 1H), 7.19-7.28 (m, 2H), 7.32-7.37 (m, 2H), 7.40-7.48 (m, 2H), 7.51-7.55 (m, 2H), 7.68 (d, 1H), 7.80-7.95 (m, 3H), 7.98 (s, 1H), 8.04 (s, 1H), 8.20 (d, 1H), 8.31 (d, 1H) ppm

### Example 14

4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid methyl ester (179 mg, 55%) was prepared according to General Procedure A using trans 4-bromo cinnamic acid (227 mg, 1 mmol) and 2-bromo-2,4-dichloro acetophenone (267 mg, 1 mmol) and obtained 2-[2-(4-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(4-Bromo-phenyl)-(E)-vinyl]-4-(2,4-dichloro-phenyl)-1H-imidazol-1-yl]-acetic acid methyl ester (466 mg, 1 mmol) was coupled with 4-ethoxy phenyl boronic acid (165 mg, 1 mmol) following General Procedure B and resulting 4{-(2,4-dichloro-phenyl)-2-[2-(4′-ethoxy-biphenyl-3-yl]-imidazol-1-yl}acetic acid methyl ester (479 mg, 1 mmol) was hydrolyzed according to General Procedure F and resulted {4-(2,4-Dichloro-phenyl)-2-[2-(4′-ethoxy-biphenyl-4-yl)-(E)-vinyl]-imidazol-1-yl}-acetic acid (247 mg, 0.5 mmol) was coupled with 4-(aminomethyl)-benzoic acid-methyl ester (83 mg, 0.5 mmol) following general procedure G. LCMS: 640 (M+H)⁺

### Example 15

4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid methyl ester (160 mg, 0.25 mmol) was hydrolyzed according to General Procedure F to give 4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid (99 mg, 63%).

LCMS: 626 (M+H)⁺

### Example 16

4-[2-[2-(4-Bromo-phenyl)-(E)-vinyl]-4-(2,4-dichloro-phenyl)-imidazol-1-ylmethyl]-benzoic acid methyl ester (300 mg, 0.55 mmol) was treated with 6-fluoro-2-methoxyphenylboronic acid using general procedure B, followed by ester hydrolysis according to general procedure F to give 4-{4-(2,4-dichloro-phenyl)-2-[2-(6′-fluoro-2′-methoxy-biphenyl-4-yl)-(E)-vinyl]-imidazol-1-ylmethyl}-benzoic acid (197 mg, 62% yield).

LCMS: m/z 573 (M+H)⁺; ¹H NMR (DMSO-d₆, 400 MHz): δ 3.74 (s, 3H), 5.62 (s, 2H), 7.08-7.20 (m, 3H), 7.30-7.37 (m, 3H), 7.48-7.53 (m, 3H), 7.56 (d, 1H), 7.63 (d, 1H), 7.69 (d, 2H), 7.93 (d, 2H), 8.10 (s, 1H), 8.27 (d, 1H) ppm.

### Example 17

4-[2-[2-(4-Bromo-phenyl)-(E)-vinyl]-4-(2,4-dichloro-phenyl)-imidazol-1-ylmethyl]-benzoic acid methyl ester (300 mg, 0.55 mmol) was treated with 3-cyanophenyl boronic acid using general procedure B, followed by ester hydrolysis according to general procedure F to give 4-[2-[2-(3′-cyano-biphenyl-4-yl)-(E)-vinyl]-4-(2,4-dichloro-phenyl)-imidazol-1-ylmethyl]-benzoic acid (53 mg, 17% yield).

LCMS: m/z 550 (M+H)⁺; ¹H NMR (DMSO-d₆, 400 MHz): δ 5.64 (s, 2H), 7.33-7.41 (m, 3H), 7.50 (dd, 1H), 7.58 (d, 1H), 7.64 (d, 1H), 7.67 (d, 1H), 7.75-7.79 (m, 4H), 7.82 (d, 1H), 7.93 (d, 2H), 8.06 (d, 1H), 8.10 (s, 1H), 8.20 (s, 1H), 8.27 (d, 1H) ppm.

### Example 18

4-Bromophenylacetic acid (2.15 g, 10 mmol) is treated according to general procedure A using 2,4-dichlorophenacyl bromide to give the intermediate 2-(4-bromo-benzyl)-4-(2,4-dichloro-phenyl)-1H-imidazole, which is then treated as described in general procedure E using methyl 4-(bromomethyl)benzoate to give 4-[2-(4-bromo-benzyl)-4-(2,4-dichloro-phenyl)-imidazol-1-ylmethyl]-benzoic acid methyl ester (1.96 g, 37% total yield). LCMS: m/z 531 (M+H)⁺

4-[4-(2,4-Dichloro-phenyl)-2-(4′-trifluoromethyl-biphenyl-4-ylmethyl)-imidazol-1-ylmethyl]-benzoic acid methyl ester (41 mg, 34% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 4-(trifluoromethyl)benzeneboronic acid (46 mg, 0.24 mmol).

LCMS: m/z 595 (M+H)⁺.

### Example 19

4-[4-(2,4-Dichloro-phenyl)-2-(4′-trifluoromethyl-biphenyl-4-ylmethyl)-imidazol-1-ylmethyl]-benzoic acid (32 mg, 91% yield) is prepared according to general procedure F using 4-[4-(2,4-dichloro-phenyl)-2-(4′-trifluoromethyl-biphenyl-4-ylmethyl)-imidazol-1-ylmethyl]-benzoic acid methyl ester (36 mg, 0.06 mmol).

LCMS: m/z 581 (M+H)⁺; ¹H NMR (DMSO-d6, 400 MHz): δ 4.10 (s, 2H), 5.34 (s, 2H), 7.13 (d, 2H), 7.23 (d, 2H), 7.40 (d, 2H), 7.44 (dd, 1H), 7.48 (d, 2H), 7.60 (d, 1H), 7.68 (d, 2H), 7.81 (d, 2H), 7.94 (s, 1H), 8.18 (d, 1H) ppm.

### Example 20

4-[4-(2,4-Dichloro-phenyl)-2-(3′-trifluoromethyl-biphenyl-4-ylmethyl)-imidazol-1-ylmethyl]-benzoic acid methyl ester (37 mg, 31% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 3-(trifluoromethyl)benzeneboronic acid (46 mg, 0.24 mmol).

LCMS: m/z 595 (M+H)⁺.

### Example 21

4-[4-(2,4-Dichloro-phenyl)-2-(3′-trifluoromethyl-biphenyl-4-ylmethyl)-imidazol-1-ylmethyl]-benzoic acid (26 mg, 89% yield) is prepared according to general procedure F using 4-[4-(2,4-dichloro-phenyl)-2-(3′-trifluoromethyl-biphenyl-4-ylmethyl)-imidazol-1-ylmethyl]-benzoic acid methyl ester (30 mg, 0.05 mmol).

LCMS: m/z 581 (M+H)⁺; ¹H NMR (DMSO-d6, 400 MHz): δ 4.12 (s, 2H), 5.35 (s, 2H), 7.14 (d, 2H), 7.26 (d, 2H), 7.44 (dd, 1H), 7.57 (d, 2H), 7.60 (d, 1H), 7.65-7.69 (m, 4H), 7.82 (d, 2H), 7.95 (s, 1H), 8.17 (d, 1H) ppm.

### Example 22

4-[4-(2,4-Dichloro-phenyl)-2-(4′-trifluoromethoxy-biphenyl-4-ylmethyl)-imidazol-1-ylmethyl]-benzoic acid methyl ester (93 mg, 78% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 4-(trifluoromethoxy)benzeneboronic acid (50 mg, 0.24 mmol).

LCMS: m/z 611 (M+H)⁺.

### Example 23

4-[4-(2,4-Dichloro-phenyl)-2-(4′-trifluoromethoxy-biphenyl-4-ylmethyl)-imidazol-1-ylmethyl]-benzoic acid (54 mg, 90% yield) is prepared according to general procedure F using 4-[4-(2,4-dichloro-phenyl)-2-(4′-trifluoromethoxy-biphenyl-4-ylmethyl)-imidazol-1-ylmethyl]-benzoic acid methyl ester (61 mg, 0.1 mmol).

LCMS: m/z 597 (M+H)⁺; ¹H NMR (DMSO-d6, 400 MHz): δ 4.11 (s, 2H), 5.34 (s, 2H), 7.13 (d, 2H), 7.23 (d, 2H), 7.39 (d, 2H), 7.43 (dd, 1H), 7.48 (d, 2H), 7.60 (d, 1H), 7.68 (d, 2H), 7.81 (d, 2H), 7.94 (s, 1H), 8.17 (d, 1H) ppm.

### Example 24

4-[4-(2,4-Dichloro-phenyl)-2-(3′-trifluoromethoxy-biphenyl-4-ylmethyl)-imidazol-1-ylmethyl]-benzoic acid methyl ester (88 mg, 72% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 3-(trifluoromethoxy)benzeneboronic acid (50 mg, 0.24 mmol).

LCMS: m/z 611 (M+H)⁺.

### Example 25

4-[4-(2,4-Dichloro-phenyl)-2-(3′-trifluoromethoxy-biphenyl-4-ylmethyl)-imidazol-1-ylmethyl]-benzoic acid (50 mg, 83% yield) is prepared according to general procedure F using 4-[4-(2,4-dichloro-phenyl)-2-(3′-trifluoromethoxy-biphenyl-4-ylmethyl)-imidazol-1-ylmethyl]-benzoic acid methyl ester (61 mg, 0.1 mmol).

LCMS: m/z 597 (M+H)⁺; ¹H NMR (DMSO-d6, 400 MHz): δ 4.14 (s, 2H), 5.37 (s, 2H), 7.13 (d, 2H), 7.24 (d, 2H), 7.44 (dd, 1H), 7.57 (d, 2H), 7.60 (d, 1H), 7.65-7.69 (m, 4H), 7.81 (d, 2H), 7.94 (s, 1H), 8.17 (d, 1H) ppm.

### Example 26

4-[4-(2,4-Dichloro-phenyl)-2-(3′-methanesulfonyl-biphenyl-4-ylmethyl)-imidazol-1-ylmethyl]-benzoic acid methyl ester (68 mg, 56% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and (3-methylsulfonylphenyl)boronic acid (48 mg, 0.24 mmol).

LCMS: m/z 605 (M+H)⁺.

### Example 27

4-[4-(2,4-Dichloro-phenyl)-2-(3′-methanesulfonyl-biphenyl-4-ylmethyl)-imidazol-1-ylmethyl]-benzoic acid (51 mg, 86% yield) is prepared according to general procedure F using 4-[4-(2,4-dichloro-phenyl)-2-(3′-methanesulfonyl-biphenyl-4-ylmethyl)-imidazol-1-ylmethyl]-benzoic acid methyl ester (61 mg, 0.1 mmol).

LCMS: m/z 591 (M+H)⁺; ¹H NMR (DMSO-d6, 400 MHz): δ 3.28 (s, 3H), 4.14 (s, 2H), 5.37 (s, 2H), 7.13 (d, 2H), 7.24 (d, 2H), 7.44 (dd, 1H), 7.57 (d, 2H), 7.60 (d, 1H), 7.65-7.69 (m, 4H), 7.81 (d, 2H), 7.94 (s, 1H), 8.17 (d, 1H) ppm.

### Example 28

4-[4-(2,4-Dichloro-phenyl)-2-(4′-methanesulfonyl-biphenyl-4-ylmethyl)-imidazol-1-ylmethyl]-benzoic acid methyl ester (74 mg, 61% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and (4-methylsulfonylphenyl)boronic acid (48 mg, 0.24 mmol).

LCMS: m/z 605 (M+H)⁺.

### Example 29

4-[4-(2,4-Dichloro-phenyl)-2-(4′-methanesulfonyl-biphenyl-4-ylmethyl)-imidazol-1-ylmethyl]-benzoic acid (53 mg, 89% yield) is prepared according to general procedure F using 4-[4-(2,4-dichloro-phenyl)-2-(4′-methanesulfonyl-biphenyl-4-ylmethyl)-imidazol-1-ylmethyl]-benzoic acid methyl ester (61 mg, 0.1 mmol).

LCMS: m/z 591 (M+H)⁺; ¹H NMR (DMSO-d6, 400 MHz): δ 3.26 (s, 3H), 4.13 (s, 2H), 5.36 (s, 2H), 7.13 (d, 2H), 7.24 (d, 2H), 7.44 (dd, 1H), 7.57 (d, 2H), 7.60 (d, 1H), 7.65 (d, 2H), 7.72 (d, 2H), 7.81 (d, 2H), 7.94 (s, 1H), 8.17 (d, 1H) ppm.

### Example 30

4-(tert-Butoxycarbonylamino-methyl)-benzoic acid (502 mg, 2 mmol) is treated according to general procedure A using 2,4-dichlorophenacyl bromide to give {4-[4-(2,4-dichloro-phenyl)-1H-imidazol-2-yl]-benzyl}-carbamic acid tert-butyl ester, which is then treated as described in general procedure E using methyl 4-(bromomethyl)benzoate to give 4-[2-[4-(tert-butoxycarbonylamino-methyl)-phenyl]-4-(2,4-dichloro-phenyl)-imidazol-1-ylmethyl]-benzoic acid methyl ester, which is then treated with hydrogen chloride in ethyl ether and then coupled with 4-methylsulphonylphenylacetic acid according to general procedure G to afford the title compound 4-[4-(2,4-dichloro-phenyl)-2-(4-{[2-(4-methanesulfonyl-phenyl)-acetylamino]-methyl}-phenyl) -imidazol-1-ylmethyl]-benzoic acid methyl ester (239 mg, 18% total yield).

LCMS: m/z 662 (M+H)⁺.

### Example 31

4-[4-(2,4-Dichloro-phenyl)-2-(4-{[2-(4-methanesulfonyl-phenyl)-acetylamino]-methyl}-phenyl)-imidazol-1-ylmethyl]-benzoic acid (92 mg, 71% yield) is prepared according to general procedure F using 4-[4-(2,4-dichloro-phenyl)-2-(4-{[2-(4-methanesulfonyl-phenyl)-acetylamino]-methyl}-phenyl)-imidazol-1-ylmethyl]-benzoic acid methyl ester (133 mg, 0.2 mmol).

LCMS: m/z 648 (M+H)⁺; ¹H NMR (DMSO-d6, 400 MHz): δ 3.16 (s, 3H), 3.51 (s, 2H), 4.25 (d, 2H), 5.38 (s, 2H), 7.13 (d, 2H), 7.24 (d, 2H), 7.46-7.58 (m, 3H), 7.60 (d, 1H), 7.65 (d, 2H), 7.72 (d, 2H), 7.81 (d, 2H), 7.94 (s, 1H), 8.15 (d, 1H) ppm.

### Example 32

Trans-4-bromocinnamic acid (2.27 g, 10 mmol) is treated according to general procedure A using 2,4-difluorophenacyl bromide to give the intermediate 2-[2-(4-bromo-phenyl)-(E)-vinyl]-4-(2,4-difluoro-phenyl)-1H-imidazole, which is then treated as described in general procedure E using methyl 4-(bromomethyl)benzoate to give 4-[2-[2-(4-bromo-phenyl)-(E)-vinyl]-4-(2,4-difluoro-phenyl)-imidazol-1-ylmethyl]-benzoic acid methyl ester (1.68 g, 33% total yield). LCMS: m/z 510 (M+H)⁺

4-{4-(2,4-Difluoro-phenyl)-2-[2-(4′-ethoxy-biphenyl-4-yl)-(E)-vinyl]-imidazol-1-ylmethyl}-benzoic acid (150 mg, 56% total yield) is prepared according to general procedure B using 4-[2-[2-(4-bromo-phenyl)-(E)-vinyl]-4-(2,4-difluoro-phenyl)-imidazol-1-ylmethyl]-benzoic acid methyl ester (255 mg, 0.5 mmol) and 4-ethoxyphenylboronic acid (100 mg, 0.6 mmol), followed by ester-hydrolysis according to general procedure F.

LCMS: m/z 537 (M+H)⁺; ¹H NMR (DMSO-d6, 400 MHz): δ 1.34 (t, 3H), 4.06 (q, 2H), 5.63 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.17 (d, 1H) ppm.

### Example 33

4-{4-(2,4-Difluoro-phenyl)-2-[2-(4′-ethoxy-biphenyl-4-yl)-ethyl]-imidazol-1-ylmethyl}-benzoic acid (18 mg, 67% yield) is prepared according to general procedure V using 4-{4-(2,4-difluoro-phenyl)-2-[2-(4′-ethoxy-biphenyl-4-yl)-(E)-vinyl]-imidazol-1-ylmethyl}-benzoic acid (27 mg, 0.05 mmol).

LCMS: m/z 539 (M+H)⁺; ¹H NMR (DMSO-d6, 400 MHz): δ 1.32 (t, 3H), 2.86 (m, 2H), 2.96 (m, 2H), 4.03 (q, 2H), 5.32 (s, 2H), 7.13 (d, 2H), 7.24 (d, 2H), 7.39 (d, 1H), 7.47 (d, 2H), 7.62 (d, 1H), 7.65-7.69 (m, 4H), 7.81 (d, 2H), 7.94 (s, 1H), 8.17 (d, 1H) ppm.

### Example 34

4-{4-(2,4-Difluoro-phenyl)-2-[2-(4′-hydroxy-biphenyl-4-yl)-(E)-viny]-imidazol-1-ylmethyl}-benzoic acid (72 mg, 71% total yield) is prepared according to general procedure C using 4-{4-(2,4-difluoro-phenyl)-2-[2-(4′-ethoxy-biphenyl-4-yl)-(E)-vinyl]-imidazol-1-ylmethyl}-benzoic acid (107 mg, 0.2 mmol).

LCMS: m/z 509 (M+H)⁺; ¹H NMR (DMSO-d6, 400 MHz): δ 5.62 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.16 (d, 1H) ppm.

### Example 35

4-[2-[2-(4′-Butoxy-biphenyl-4-yl)-(E)-vinyl]-4-(2,4-difluoro-phenyl)-imidazol-1-ylmethyl]-benzoic acid (28 mg, 49% total yield) is prepared according to general procedure E using 4-{4-(2,4-difluoro-phenyl)-2-[2-(4′-hydroxy-biphenyl-4-yl)-(E)-vinyl]-imidazol-1-ylmethyl}-benzoic acid (51 mg, 0.1 mmol) and 1-bromobutane, followed by ester-hydrolysis according to general procedure F.

LCMS: m/z 565 (M+H)⁺; ¹H NMR (DMSO-d6, 400 MHz): δ 1.04 (t, 3H), 1.46 (m, 2H), 1.90 (m, 2H), 4.18 (t, 2H), 5.61 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.17 (d, 1H) ppm.

### Example 36

4-{4-(2,4-Difluoro-phenyl)-2-[2-(3′-trifluoromethyl-biphenyl-4-yl)-(E)-vinyl]-imidazol-1-ylmethyl}-benzoic acid (87 mg, 31% total yield) is prepared according to general procedure B using 4-[2-[2-(4-bromo-phenyl)-(E)-vinyl]-4-(2,4-difluoro-phenyl)-imidazol-1-ylmethyl]-benzoic acid methyl ester (255 mg, 0.5 mmol) and 3-(trifluoromethyl)benzeneboronic acid (114 mg, 0.6 mmol), followed by ester-hydrolysis according to general procedure F.

LCMS: m/z 561 (M+H)⁺; ¹H NMR (DMSO-d6, 400 MHz): δ 5.60 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.18 (d, 1H) ppm.

### Example 37

4-{4-(2,4-Difluoro-phenyl)-2-[2-(3′-trifluoromethyl-biphenyl-4-yl)-ethyl]-imidazol-1-ylmethyl}-benzoic acid (21 mg, 74% yield) is prepared according to general procedure V using 4-{4-(2,4-difluoro-phenyl)-2-[2-(3′-trifluoromethyl-biphenyl-4-yl)-(E)-vinyl]-imidazol-1-ylmethyl}-benzoic acid (28 mg, 0.05 mmol).

LCMS: m/z 563 (M+H)⁺; ¹H NMR (DMSO-d6, 400 MHz): δ 2.88 (m, 2H), 2.97 (m, 2H), 5.32 (s, 2H), 7.13 (d, 2H), 7.24 (d, 2H), 7.39 (d, 1H), 7.47 (d, 2H), 7.62 (d, 1H), 7.65-7.69 (m, 4H), 7.81 (d, 2H), 7.94 (s, 1H), 8.17 (d, 1H) ppm.

### Example 38

4-(2,4-Dichloro-phenyl)-2-[2-(4-nitro-phenyl)-(E)-vinyl]-1H-imidazole (1.98 g, 5.5. mmol) was treated with methyl 4-bromomethyl benzoate using general procedure E to provide 4-{4-(2,4-dichloro-phenyl)-2-[2-(4-nitro-phenyl)-(E)-vinyl]-imidazol-1-ylmethyl}-benzoic acid methyl ester (753 mg, 27% yield). 30 mg (0.059 mmol) of the ester was hydrolyzed according to general procedure F to provide 4-{4-(2,4-dichloro-phenyl)-2-[2-(4-nitro-phenyl)-(E)-vinyl]-imidazol-1-ylmethyl}-benzoic acid (24 mg, 82% yield).

LCMS: m/z 494 (M+H)+; 1H NMR (CD3OD, 400 MHz): δ 5.53 (s, 2H), 7.18 (d, 1H), 7.31 (d, 2H), 7.38 (dd, 1H), 7.49 (d, 1H), 7.65-7.72 (m, 3H), 7.79 (s, 1H), 8.06 (m, 3H), 8.23 (d, 2H) ppm.

### Example 39

4-{4-(2,4-Dichloro-phenyl)-2-[2-(4-nitro-phenyl)-(E)-vinyl]-imidazol-1-ylmethyl}-benzoic acid methyl ester (453 mg, 0.89 mmol) was reduced according to general procedure K to provide 4-[2-[2-(4-amino-phenyl)-(E)-vinyl]-4-(2,4-dichloro-phenyl)-imidazol-1-ylmethyl]-benzoic acid methyl ester (350 mg, 82% yield).

LCMS: m/z 478 (M+H)⁺.

### Example 40

4-[2-[2-(4-Amino-phenyl)-(E)-vinyl]-4-(2,4-dichloro-phenyl)-imidazol-1-ylmethyl]-benzoic acid methyl ester (17 mg, 0.036 mmol) was hydrolyzed according to general procedure F to provide 4-[2-[2-(4-amino-phenyl)-(E)-vinyl]-4-(2,4-dichloro-phenyl)-imidazol-1-ylmethyl]-benzoic acid (5.4 mg, 33% yield).

LCMS: m/z 464 (M+H)⁺; ¹H NMR (DMSO, 400 MHz): δ 5.52 (s, 2H), 6.54 (d, 2H), 6.90 (d, 1H), 7.25-7.34 (m, 4H), 7.38 (d, 1H), 7.49 (dd, 1H), 7.63 (d, 1H), 7.90 (d, 2H), 8.05 (s, 1H), 8.27 (d, 1H) ppm.

### Example 41

4-[2-[2-(4-Amino-phenyl)-(E)-vinyl]-4-(2,4-dichloro-phenyl)-imidazol-1-ylmethyl]-benzoic acid methyl ester (69 mg, 0.14 mmol) was treated with n-butanesulfonyl chloride according to general procedure L to provide 4-[2-{2-[4-(butane-1-sulfonylamino)-phenyl]-(E)-vinyl}-4-(2,4-dichloro-phenyl)-imidazol-1-ylmethyl]-benzoic acid methyl ester (48 mg, 57% yield).

LCMS: m/z 598 (M+H)⁺; ¹H NMR (CDCl₃, 400 MHz): δ 0.90 (t, 3H), 1.42 (m, 2H), 1.80 (m, 2H), 3.10 (m, 2H), 3.93 (s, 3H), 5.34 (s, 2H), 6.66 (s, 1H), 6.73 (d, 1H), 7.17 (d, 2H), 7.23 (d, 2H), 7.34 (dd, 1H), 7.41 (d, 2H), 7.43 (d, 1H), 7.64 (d, 1H), 7.71 (s, 1H), 8.05 (d, 2H), 8.26 (d, 1H) ppm.

### Example 42

4-[2-{2-[4-(Butane-1-sulfonylamino)-phenyl]-(E)-vinyl}-4-(2,4-dichloro-phenyl)-imidazol-1-ylmethyl]-benzoic acid methyl ester (45 mg, 0.075 mmol) was hydrolyzed according to general procedure F to provide 4-[2-{2-[4-(butane-1-sulfonylamino)-phenyl]-(E)-vinyl}-4-(2,4-dichloro-phenyl)-imidazol-1-ylmethyl]-benzoic acid (30 mg, 68% yield).

LCMS: m/z 584 (M+H)⁺; ¹H NMR (DMSO, 400 MHz): δ 0.83 (t, 3H), 1.35 (m, 2H), 1.64 (m, 2H), 3.12 (m, 2H), 5.60 (s, 2H), 6.66 (s, 1H), 7.17-7.23 (m, 3H), 7.34 (d, 2H), 7.46-7.53 (m, 2H), 7.62 (d, 2H), 7.65 (d, 1H), 7.93 (d, 2H), 8.09 (s, 1H), 8.28 (d, 1H), 9.93 (br s, 1H), 12.82 (br s, 1H) ppm.

### Example 43

4-[2-[2-(4-Amino-phenyl)-(E)-vinyl]-4-(2,4-dichloro-phenyl)-imidazol-1-ylmethyl]-benzoic acid methyl ester (71 mg, 0.15 mmol) was treated with 4-n-butylbenzenesulfonyl chloride according to general procedure L to provide 4-[2-{2-[4-(4-butyl-benzenesulfonylamino)-phenyl]-(E)-vinyl}-4-(2,4-dichloro-phenyl)-imidazol-1-ylmethyl]-benzoic acid methyl ester (95 mg, 93% yield).

LCMS: m/z 674 (M+H)⁺; ¹H NMR (CDCl₃, 400 MHz): δ 0.90 (t, 3H), 1.30 (m, 2H), 1.57 (m, 2H), 2.62 (t, 2H), 3.92 (s, 3H), 5.31 (s, 2H), 6.69 (d, 1H), 6.98-7.05 (m, 3H), 7.21 (m, 4H), 7.28-7.33 (m, 3H), 7.42 (d, 1H), 7.58 (d, 1H), 7.68 (m, 3H), 8.03 (d, 2H), 8.24 (d, 1H) ppm.

### Example 44

4-[2-{2-[4-(4-Butyl-benzenesulfonylamino)-phenyl]-(E)-vinyl}-4-(2,4-dichloro-phenyl)-imidazol-1-ylmethyl]-benzoic acid methyl ester (92 mg, 0.14 mmol) was hydrolyzed according to general procedure F to provide 4-[2-{2-[4-(4-butyl-benzenesulfonylamino)-phenyl]-(E)-vinyl}-4-(2,4-dichloro-phenyl)-imidazol-1-ylmethyl]-benzoic acid (82 mg, 91% yield).

LCMS: m/z 660 (M+H)⁺; ¹H NMR (DMSO, 400 MHz): δ 0.85 (t, 3H), 1.26 (m, 2H), 1.51 (m, 2H), 2.60 (t, 2H), 5.57 (s, 2H), 7.09 (d, 2H), 7.15 (d, 1H), 7.33 (d, 2H), 7.37 (d, 2H), 7.42 (d, 1H), 7.48-7.54 (m, 3H), 7.64 (d, 1H), 7.69 (d, 2H) 7.92 (d, 2H), 8.07 (s, 1H), 8.25 (d, 1H), 10.40 (S, 1H), 12.94 (br s, 1H) ppm.

### Example 45

4-[2-[2-(4-Amino-phenyl)-(E)-vinyl]-4-(2,4-dichloro-phenyl)-imidazol-1-ylmethyl]-benzoic acid methyl ester (70 mg, 0.15 mmol) was treated with 4-n-butylbenzaldehyde according to general procedure U to provide 4-[2-{2-[4-(4-butyl-benzylamino)-phenyl]-(E)-vinyl}-4-(2,4-dichloro-phenyl)-imidazol-1-ylmethyl]-benzoic acid methyl ester (59 mg, 63% yield).

LCMS: m/z 624 (M+H)⁺; ¹H NMR (CDCl₃, 400 MHz): δ 0.92 (t, 3H), 1.35 (m, 2H), 1.58 (m, 2H), 2.60 (t, 2H), 3.90 (s, 3H), 4.29 (s, 2H), 5.28 (s, 2H), 6.54-6.60 (m, 3H), 7.15 (d, 2H), 7.20-7.30 (m, 6H), 7.32 (dd, 1H), 7.41 (d, 1H), 7.59 (d, 1H), 7.65 (s, 1H), 8.03 (d, 2H), 8.29 (d, 1H) ppm.

### Example 46

4-[2-{2-[4-(4-Butyl-benzylamino)-phenyl]-(E)-vinyl}-4-(2,4-dichloro-phenyl)-imidazol-1-ylmethyl]-benzoic acid methyl ester (55 mg, 0.09 mmol) was hydrolyzed according to general procedure F to provide 4-[2-{2-[4-(4-butyl-benzylamino)-phenyl]-(E)-vinyl}-4-(2,4-dichloro-phenyl)-imidazol-1-ylmethyl]-benzoic acid (39 mg, 72% yield).

LCMS: m/z 610 (M+H)⁺; ¹H NMR (DMSO, 400 MHz): δ 0.90 (t, 3H), 1.29 (m, 2H), 1.53 (m, 2H), 2.55 (t, 2H), 4.24 (d, 2H), 5.55 (s, 2H), 6.56 (d, 2H), 6.89 (d, 1H), 7.13 (d, 2H), 7.25 (d, 2H), 7.31-7.40 (m, 5H), 7.49 (dd, 1H), 7.63 (d, 1H), 7.92 (d, 2H), 8.02 (s, 1H), 8.27 (d, 1H), 12.95 (br s, 1H) ppm.

### Example 47

4-[2-{2-[4-(4-Butyl-benzenesulfonylamino)-phenyl]-(E)-vinyl}-4-(2,4-dichloro-phenyl)-imidazol-1-ylmethyl]-benzoic acid (16 mg, 0.024 mmol) was reduced according to general procedure V to provide 4-[2-{2-[4-(4-butyl-benzenesulfonylamino)-phenyl]-ethyl}-4-(2,4-dichloro-phenyl)-imidazol-1-ylmethyl]-benzoic acid (8 mg, 50% yield).

LCMS: m/z 662 (M+H)⁺; ¹H NMR (CD₃OD, 400 MHz): δ 0.89 (t, 3H), 1.28 (m, 2H), 1.50 (m, 2H), 2.55 (t, 2H), 2.86 (m, 4H), 4.96 (s, 2H), 6.92 (d, 2H), 6.97 (d, 2H), 7.09 (d, 2H), 7.22 (d, 2H), 7.38 (dd, 1H), 7.51 (d, 1H), 7.58 (s, 1H), 7.63 (d, 2H) 7.88 (d, 1H), 7.97 (d, 2H) ppm.

### Example 48

4-[2-[2-(4-Amino-phenyl)-(E)-vinyl]-4-(2,4-dichloro-phenyl)-imidazol-1-ylmethyl]-benzoic acid methyl ester (66 mg, 0.14 mmol) was treated with 3-trifluoromethylbenzenesulfonyl chloride according to general procedure L to provide 4-(4-(2,4-dichloro-phenyl)-2-{2-[4-(3-trifluoromethyl-benzenesulfonylamino)-phenyl]-(E)-vinyl}-imidazol-1-ylmethyl)-benzoic acid methyl ester (87 mg, 92% yield).

LCMS: m/z 686 (M+H)⁺; ¹H NMR (CDCl₃, 400 MHz): δ 3.92 (s, 3H), 5.34 (s, 2H), 6.67 (br s, 1H), 6.71 (d, 1H), 7.03 (d, 2H), 7.22 (d, 2H), 7.31-7.36 (m, 3H), 7.43 (d, 1H), 7.56-7.62 (m, 2H), 7.70 (s, 1H), 7.80 (d, 1H), 7.91 (d, 1H), 8.01-8.06 (m, 3H), 8.24 (d, 1H) ppm.

### Example 49

4-[2-[2-(4-Amino-phenyl)-(E)-vinyl]-4-(2,4-dichloro-phenyl)-imidazol-1-ylmethyl]-benzoic acid methyl ester (66 mg, 0.14 mmol) was treated with 4-trifluoromethylbenzenesulfonyl chloride according to general procedure L to provide 4-(4-(2,4-dichloro-phenyl)-2-{2-[4-(4-trifluoromethyl-benzenesulfonylamino)-phenyl]-(E)-vinyl}-imidazol-1-ylmethyl)-benzoic acid methyl ester (87 mg, 92% yield).

LCMS: m/z 686 (M+H)⁺; ¹H NMR (CDCl₃, 400 MHz): δ 3.92 (s, 3H), 5.33 (s, 2H), 6.69-6.73 (m, 2H), 7.04 (d, 2H), 7.22 (d, 2H), 7.31-7.36 (m, 3H), 7.43 (d, 1H), 7.60 (d, 1H), 7.71 (m, 3H), 7.88 (d, 2H), 8.04 (d, 2H), 8.24 (d, 1H) ppm.

### Example 50

4-(4-(2,4-Dichloro-phenyl)-2-{2-[4-(3-trifluoromethyl-benzenesulfonylamino)-phenyl]-(E)-vinyl}-imidazol-1-ylmethyl)-benzoic acid methyl ester (79 mg, 0.12 mmol) was hydrolyzed according to general procedure F to provide 4-(4-(2,4-dichloro-phenyl)-2-{2-[4-(3-trifluoromethyl-benzenesulfonylamino)-phenyl]-(E)-vinyl}-imidazol-1-ylmethyl)-benzoic acid (46 mg, 59% yield).

LCMS: m/z 672 (M+H)⁺; ¹H NMR (DMSO, 400 MHz): δ 5.58 (s, 2H), 7.09 (d, 2H), 7.18 (d, 1H), 7.33 (d, 2H), 7.43 (d, 1H), 7.50 (dd, 1H), 7.56 (d, 2H), 7.64 (d, 1H), 7.82 (t, 1H) 7.93 (d, 2H), 8.01-8.06 (m, 3H), 8.08 (s, 1H), 8.25 (d, 1H), 10.59 (s, 1H), 12.96 (br s, 1H) ppm.

### Example 51

4-(4-(2,4-Dichloro-phenyl)-2-{2-[4-(4-trifluoromethyl-benzenesulfonylamino)-phenyl]-(E)-vinyl}-imidazol-1-ylmethyl)-benzoic acid methyl ester (79 mg, 0.12 mmol) was hydrolyzed according to general procedure F to provide 4-(4-(2,4-dichloro-phenyl)-2-{2-[4-(4-trifluoromethyl-benzenesulfonylamino)-phenyl]-(E)-vinyl}-imidazol-1-ylmethyl)-benzoic acid (54 mg, 70% yield).

LCMS: m/z 672 (M+H)⁺; ¹H NMR (DMSO, 400 MHz): δ 5.59 (s, 2H), 7.10 (d, 2H), 7.17 (d, 1H), 7.33 (d, 2H), 7.43 (d, 1H), 7.49 (dd, 1H), 7.55 (d, 2H), 7.64 (d, 1H), 7.92 (d, 2H) 7.97 (s, 4H), 8.08 (s, 1H), 8.25 (d, 1H), 10.68 (br s, 1H), 12.96 (br s, 1H) ppm.

### Example 52

4-[2-[2-(4-Amino-phenyl)-(E)-vinyl]-4-(2,4-dichloro-phenyl)-imidazol-1-ylmethyl]-benzoic acid methyl ester (35 mg, 0.073 mmol) was treated with p-toluenesulfonyl chloride according to general procedure L to provide 4-(4-(2,4-dichloro-phenyl)-2-{2-[4-(toluene-4-sulfonylamino)-phenyl]-(E)-vinyl}-imidazol-1-ylmethyl)-benzoic acid methyl ester (39 mg, 84% yield).

LCMS: m/z 632 (M+H)⁺; ¹H NMR (CDCl₃, 400 MHz): δ 2.36 (s, 3H), 3.90 (s, 3H), 5.30 (s, 2H), 6.68 (d, 1H), 7.03 (d, 2H), 7.20 (d, 4H), 7.26-7.32 (m, 3H), 7.41 (d, 1H), 7.57 (d, 1H), 7.65 (d, 2H), 7.68 (s, 1H), 8.03 (d, 2H), 8.23 (d, 1H) ppm.

### Example 53

4-(4-(2,4-Dichloro-phenyl)-2-{2-[4-(toluene-4-sulfonylamino)-phenyl]-(E)-vinyl}-imidazol-1-ylmethyl)-benzoic acid methyl ester (36 mg, 0.057 mmol) was hydrolyzed according to general procedure F to provide 4-(4-(2,4-dichloro-phenyl)-2-{2-[4-(toluene-4-sulfonylamino)-phenyl]-(E)-vinyl}-imidazol-1-ylmethyl)-benzoic acid (26 mg, 74% yield).

LCMS: m/z 618 (M+H)⁺; ¹H NMR (CD₃OD, 400 MHz): δ 2.33 (s, 3H), 5.45 (s, 2H), 6.95 (d, 1H), 7.07 (d, 2H), 7.23 (d, 2H), 7.28 (d, 2H), 7.36 (m, 3H), 7.43 (d, 1H), 7.48 (d, 1H), 7.63 (d, 2H) 7.77 (s, 1H), 7.95-8.00 (m, 3H) ppm.

### Example 54

4-[2-{2-[4-(4-Butyl-benzenesulfonylamino)-phenyl]-(E)-vinyl}-4-(2,4-dichloro-phenyl)-imidazol-1-ylmethyl]-benzoic acid (24 mg, 0.036 mmol) was treated with sodium hydride and methyl iodide according to general procedure P, then the methyl ester which formed was hydrolyzed according to general procedure F to provide 4-[2-(2-{4-[(4-butyl-benzenesulfonyl)-methyl-amino]-phenyl}-(E)-vinyl)-4-(2,4-dichloro-phenyl)-imidazol-1-ylmethyl]-benzoic acid (11 mg, 45% yield).

LCMS: m/z 674 (M+H)⁺; ¹H NMR (CD₃OD, 400 MHz): δ 0.95 (t, 3H), 1.38 (m, 2H), 1.64 (M, 2H), 2.70 (t, 2H), 3.18 (s, 3H), 5.48 (s, 2H), 6.95 (d, 1H), 7.09 (d, 2H), 7.28-7.33 (m, 4H), 7.37 (dd, 1H), 7.43-7.49 (m, 5H), 7.58 (d, 1H) 7.74 (s, 1H), 8.03-8.09 (m, 3H) ppm.

### Example 55

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethyl)-phenyl boronic acid (189 mg, 1 mmol) following General Procedure B to give 4-{4-(2,4-dichloro-phenyl)-2-[2-(4′-trifluoromethyl-biphenyl-4-yl)-(E)-vinyl]imidazol-1-yl-methyl} benzoic acid methyl ester (313 mg, 51%).

LCMS: 607 (M+H)⁺.

### Example 56

4-{4-(2,4-dichloro-phenyl)-2-[2-(4′-trifluoromethyl-biphenyl-4-yl)-(E)-vinyl]-imidazol-1-yl-methyl} benzoic acid methyl ester (303 mg, 0.5 mmol) was hydrolyzed according to General Procedure F to give 4-{4-(2,4-Dichloro-phenyl)-2[2-(4′-trifluoromethyl-biphenyl-4-yl)-(E)-vinyl]-imidazol-1-ylmethyl}-benzoic acid (197 mg, 67%).

LCMS: 593 (M+H)^{+ 1}H NMR (DMSO, 400 MHz): δ 5.82 (s, 2H), 7.48-7.50 (m, 2H), 7.56 (s, 1H), 7.60-7.64 (m, 3H), 7.81-7.88 (m, 4H), 7.91-7.99 (m, 4H), 8.14-8.19 (m, 3H), 8.32 (s, 1H) ppm.

### Example 57

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethoxy)-phenyl boronic acid (205 mg, 1 mmol) following General Procedure B to give 4-{4-(2,4-dichloro-phenyl)-2-[2-(4′-trifluoromethoxy-biphenyl-4-yl)-(E)-vinyl]-imidazol-1-yl-methyl} benzoic acid methyl ester (324 mg, 52%).

LCMS: 623 (M+H)⁺

### Example 58

4-{4-(2,4-dichloro-phenyl)-2-[2-(4′-trifluoromethoxy-biphenyl-4-yl)-(E)-vinyl]-imidazol-1-yl-methyl} benzoic acid methyl ester (311 mg, 0.5 mmol) was hydrolyzed according to General Procedure F to give 4-{4-(2,4-Dichloro-phenyl)-2[2-(4′-trifluoromethoxy-biphenyl-4-yl)-(E)-vinyl]-imidazol-1-ylmethyl}-benzoic acid (198 mg, 65%).

LCMS: 609 (M+H)^{+ 1}H NMR (DMSO, 400 MHz): δ 5.66 (s, 2H), 7.36-7.40 (m, 2H), 7.44-7.46 (m, 2H), 7.51 (d, 1H), 7.52 (d, 1H), 7.53 (d, 1H), 7.59 (s, 1H), 7.63-7.66 (m, 2H), 7.70-7.72 (m, 2H), 7.76-7.84 (m, 2H), 7.93-7.95 (m. 2H), 8.13 (s, 1H), 8.27 (d, 1H) ppm.

### Example 59

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-butoxy-phenyl boronic acid (195 mg, 1 mmol) following General Procedure B to give 4-2-[2-(4′-butoxy-biphenyl-4-yl)-(E)-vinyl]-4-{4-(2,4-dichloro-phenyl)-imidazol-1-yl-methyl} benzoic acid methyl ester (315 mg, 51%).

LCMS: 611 (M+H)⁺.

### Example 60

4-2-[2-(4′-butoxy-biphenyl-4-yl)-(E)-vinyl]-4-{4-(2,4-dichloro-phenyl)-imidazol-1-yl-methyl} benzoic acid methyl ester (305 mg, 0.5 mmol) was hydrolyzed according to General Procedure F to give 4-[2-[2-(4′-Butoxy-biphenyl-4-yl)-(E)-vinyl]-4-(2,4-dichloro-phenyl)-imidazol-1-ylmethyl]-benzoic acid (198 mg, 66%)

LCMS: 597 (M+H)^{+ 1}H NMR (DMSO, 400 MHz): δ 0.96 (t, 3H), 1.43-1.45 (m, 2H), 1.69-1.73 (m, 2H), 4.02 (q, 2H), 5.64 (s, 2H), 7.02 (d, 1H), 7.29 (s, 1H), 7.33-7.37 (m, 4H), 7.52-7.54 (m, 4H), 7.58-7.64 (m, 4H), 7.65 (d, 1H), 7.92 (d, 1H), 8.10 (s, 1H), 8.27 (d, 1H) ppm.

### Example 61

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(trifluoromethyl)-phenyl boronic acid (189 mg, 1 mmol) following General Procedure B to give 4-{4-(2,4-dichloro-phenyl)-2-[2-(3′-trifluoromethyl-biphenyl-4-yl)-(E)-vinyl]-imidazol-1-yl-methyl} benzoic acid methyl ester (312 mg, 52%).

LCMS: 607 (M+H)^{+ 1}H NMR (CDCl₃, 400 MHz): δ 3.91 (s, 3H), 5.37 (s, 2H) 6.87 (d, 1H), 7.33-7.7.36 (m, 4H), 7.43 (d, 1H), 7.53 (s, 1H), 7.55-7.61 (m, 4H), 7.72-7.75 (m, 4H), 7.83 (s, 1H), 8.05 (s, 1H), 8.30 (d, 1H) ppm.

### Example 62

4-{4-(2,4-Dichloro-phenyl)-2-[2-(3′-trifluoromethyl-biphenyl-4-yl)-(E)-vinyl]-imidazol-1yl-methyl} benzoic acid methyl ester (303 mg, 0.5 mmol) was hydrolyzed according to General Procedure F to give 4-{4-(2,4-Dichloro-phenyl)-2[2-(3′-trifluoromethyl-biphenyl-4-yl)-(E)-vinyl]-imidazol-1-ylmethyl}-benzoic acid (197 mg, 67%).

LCMS: 593 (M+H)^{+ 1}H NMR (DMSO, 400 MHz): δ 5.70 (s, 2H), 7.40-7.42 (m, 4H), 7.47 (s, 1H), 7.55 (d, 2H), 7.71 (d, 2H), 7.81 (s, 1H), 7.94 (d, 2H), 8.01-8.04 (m, 2H), 8.18-8.22 (m, 4H) ppm.

### Example 63

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethoxy)-phenyl boronic acid (205 mg, 1 mmol) following General Procedure B to give 4-{4-(2,4-dichloro-phenyl)-2-[2-(3′-trifluoromethoxy-biphenyl-4-yl)-(E)-vinyl]-imidazol-1yl-methyl} benzoic acid methyl ester (321 mg, 51%).

LCMS: 623 (M+H)⁺.

### Example 64

4-{4-(2,4-dichloro-phenyl)-2-[2-(3′-trifluoromethoxy-biphenyl-4-yl)-(E)-vinyl]-imidazol-1yl-methyl} benzoic acid methyl ester (311 mg, 0.5 mmol) was hydrolyzed according to General Procedure F to give 4-{4-(2,4-Dichloro-phenyl)-2[2-(4′-trifluoromethoxy-biphenyl-4-yl)-(E)-vinyl]-imidazol-1-ylmethyl}-benzoic acid (198 mg, 65%).

LCMS: 609 (M+H)^{+ 1}H NMR (DMSO, 400 MHz): δ 4.81 (s, 2H), 6.51-6.55 (m, 2H), 6.66 (d, 2H), 6.72-6.75 (m, 4H), 6.76 (s, 1H), 6.77 (s, 1H), 6.81-6.93 (m, 4H), 7.10 (d, 2H), 7.27 (s, 1H), 7.45 (d, 1H) ppm.

### Example 65

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-amino-phenyl boronic acid (137 mg, 1 mmol) following General Procedure B and obtained 4-{4-(2,4-dichloro-phenyl)-2-[2-(3′-amino-biphenyl-4-yl)-(E)-viny]-imidazol-1-yl-methyl}benzoic acid methyl ester (277 mg, 0.5 mmol) was alkylated according to General Procedure P to give 4-{4-(2,4-Dichloro-phenyl)-2[2-(3-trifluoromethanesulfonylamino -biphenyl-4-yl)-(E)-vinyl]imidazol-1-ylmethyl}-benzoic acid (228 mg, 66%).

LCMS: 686 (M+H)⁺.

### Example 66

4-{4-(2,4-Dichloro-phenyl)-2-[2-(3′-trifluoromethanesulfonylamino-biphenyl-4-yl)-(E)-vinyl]-imidazol-1-ylmethyl}-benzoic acid methyl ester (343 mg, 0.5 mmol) was hydrolyzed according to General Procedure F to give 4-{4-(2,4-Dichloro-phenyl)-2-[2-(3′-trifluoromethanesulfonylamino-biphenyl-4-yl)-(E)-vinyl]-imidazol-1-ylmethyl}-benzoic acid (238 mg, 70%).

LCMS: 672 (M+H)^{+ 1}H NMR (DMSO, 400 MHz): δ 5.61 (s, 2H), 6.93 (d, 1H), 7.05 (d, 1H), 7.12-7.14 (m, 2H), 7.24 (s, 1H), 7.30-7.34 (m, 4H), 7.50-7.57 (m, 4H), 7.64 (s, 1H), 7.70 (d, 1H), 7.92 (d, 2H), 8.10 (s, 1H), 8.30 (d, 1H) ppm.

### Example 67

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with (4-Bromomethyl-phenyl)-acetic acid methyl ester (243 mg, 1 mmol) following general procedure E. The resulted {-4-[2-[2-(4-Bromo-phenyl)-(E)-vinyl]-4-(2,4-dichloro-phenyl)-imidazol-1-ylmethyl]-phenyl}-acetic acid methyl ester (556 mg, 1 mmol) was coupled with 3-methanesulfonyl-phenyl boronic acid (200 mg, 1 mmol) following General Procedure B to give (4-{4-(2,4-Dichloro-phenyl)-2-[2-(3′-methanesulfonyl-biphenyl-4-yl)-(E)-vinyl]-imidazol-1-ylmethyl}-phenyl) -acetic acid methyl ester (321 mg, 50%).

LCMS: 631 (M+H)⁺

### Example 68

(4-{4-(2,4-Dichloro-phenyl)-2-[2-(3′-methanesulfonyl-biphenyl-4-yl)-(E)-vinyl]-imidazol-1-ylmethyl}-phenyl)-acetic acid methyl ester (315 mg, 0.5 mmol) was hydrolyzed according to General Procedure F to give (4-{4-(2,4-Dichloro-phenyl)-2-[2-(3′-methanesulfonyl-biphenyl-4-yl)-(E)-vinyl]-imidazol-1-ylmethyl}-phenyl) -acetic acid (198 mg, 64%).

LCMS: 617 (M+H)^{+ 1}H NMR (DMSO, 400 MHz): δ 3.31 (s, 3H), 3.46 (s, 2H), 5.51 (s, 2H), 7.23 (s, 1H), 7.45-7.49 (m, 2H), 7.51-7.57 (m, 2H), 7.61-7.64 (m, 2H), 7.75-7.76 (m, 2H), 7.79-7.82 (m, 2H), 7.84-8.07 (m, 4H), 8.10 (d, 1H), 8.19 (s, 1H), 8.25 (d, 1H) ppm.

### Example 69

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-ethoxy-phenyl boronic acid (165 mg, 1 mmol) following General Procedure B to give 4-2-[2-(4′-ethoxy-biphenyl-4-yl)-(E)-vinyl]-4-{4-(2,4-dichloro-phenyl)-imidazol-1yl-methyl} benzoic acid methyl ester (305 mg, 52%).

LCMS: 583 (M+H)⁺.

### Example 70

4-2-[2-(4′-ethoxy-biphenyl-4-yl)-(E)-vinyl]-4-{4-(2,4-dichloro-phenyl)-imidazol-1-yl-methyl} benzoic acid methyl ester (292 mg, 0.5 mmol) was hydrolyzed according to General Procedure F to give 4-[2-[2-(4′-ethoxy-biphenyl-4-yl)-(E)-vinyl]-4-(2,4-dichloro-phenyl)-imidazol-1-ylmethyl]-benzoic acid (198 mg, 69%)

LCMS: 569 (M+H)^{+ 1}H NMR (DMSO, 400 MHz): δ 0.96 (t, 3H), 4.02 (q, 2H), 5.64 (s, 2H), 7.02 (d, 1H), 7.29 (s, 1H), 7.33-7.37 (m, 4H), 7.52-7.54 (m, 4H), 7.58-7.64 (m, 4H), 7.65 (d, 1H), 7.92 (d, 1H), 8.10 (s, 1H), 8.27 (d, 1H) ppm.

### Example 71

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B to give 4-2-[2-(4′-hydroxy-biphenyl-4-yl)-(E)-vinyl]-4-{4-(2,4-dichloro-phenyl)-imidazol-1yl-methyl} benzoic acid methyl ester (288 mg, 54%)

LCMS: 556 (M+H)⁺4-2-[2-(4′-hydroxy-biphenyl-4-yl)-(E)-vinyl]-4-{4-(2,4-dichloro-phenyl)-imidazol-1yl-methyl} benzoic acid methyl ester (278 mg, 0.5 mmol) was hydrolyzed according to General Procedure F to give 4-[2-[2-(4′-hydroxy-biphenyl-4-yl)-(E)-vinyl]-4-(2,4-dichloro-phenyl)-imidazol-1-ylmethyl]-benzoic acid (168 mg, 62%)

LCMS: 541 (M+H)^{+ 1}H NMR (DMSO, 400 MHz): δ 5.68 (s, 2H), 7.12 (d, 1H), 7.36 (s, 1H), 7.37-7.40 (m, 4H), 7.52-7.54 (m, 4H), 7.58-7.64 (m, 4H), 7.66 (d, 1H), 7.91 (d, 1H), 8.09 (s, 1H), 8.21 (d, 1H) ppm.

### Example 72

Trans 5-bromo 2-methoxy cinnamic acid (257 mg, 1 mmol) was reacted with 2-bromo-2,4-dichloro acetophenone (267 mg, 1 mmol) according to general procedure A and obtained 2-[2-(5-Bromo-2-methoxy-phenyl)-(E)-vinyl]-4-(2,4-dichloro-phenyl)-1H-imidazole (424 mg, 1 mmol) was N-alkylated with methyl-4-(bromomethyl)benzoate (229 mg, 1 mmol) following general procedure E. The resulted 4-[2-[2-(5-Bromo-2-methoxy-phenyl)-(E)-vinyl]-4-(2,4-dichloro-phenyl)-imidazol-1-ylmethyl]-benzoic acid methyl ester (572 mg, 1 mmol) was coupled with 4-ethoxy-phenyl boronic acid (165 mg, 1 mmol) following General Procedure B to give 4-{4-(2,4-Dichloro-phenyl)-2-[2-(4′-ethoxy-4-methoxy-biphenyl-3-yl)-(E)-viny]-imidazol-1-ylmethyl}-benzoic acid methyl ester (298 mg, 49%).

LCMS: 613 (M+H)⁺.

### Example 73

4-{4-(2,4-Dichloro-phenyl)-2-[2-(4′-ethoxy-4-methoxy-biphenyl-3-yl)-(E)-vinyl]-imidazol-1-ylmethyl}-benzoic acid methyl ester (154 mg, 0.25 mmol) was hydrolyzed according to General Procedure F to give 4-{4-(2,4-Dichloro-phenyl)-2-[2-(4′-ethoxy-4-methoxy-biphenyl-3-yl)-(E)-vinyl]-imidazol-1-ylmethyl}-benzoic acid (117 mg, 78%).

LCMS: 599 (M+H)⁺. ¹H NMR (DMSO, 400 MHz): δ 1.39 (t, 3H), 3.90 (s, 3H), 4.24 (q, 2H), 5.28 (d, 2H), 7.09 (d, 2H), 7.11-7.21 (m, 2H), 7.28-7.36 (m, 2H), 7.38 (d, 1H), 7.41-7.56 (m, 4H), 7.71 (d, 1H), 7.76-8.02 (m. 4H), 8.16 (d, 1H) ppm

### Example 74

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with (4-Bromomethyl-phenyl)-acetic acid methyl ester (243 mg, 1 mmol) following general procedure E. The resulted {4-[2-[2-(4-Bromo-phenyl)-(E)-vinyl]-4-(2,4-dichloro-phenyl)-imidazol-1-ylmethyl]-phenyl}-acetic acid methyl ester (556 mg, 1 mmol) was coupled with 3-trifluoromethyl-phenyl boronic acid (189 mg, 1 mmol) following General Procedure B to give (4-{4-(2,4-Dichloro-phenyl)-2-[2-(3′-trifluoromethyl-biphenyl-4-yl)-(E)-vinyl]-imidazol-1-ylmethyl}-phenyl) -acetic acid methyl ester (321 mg, 51%).

LCMS: 621 (M+H)⁺

### Example 75

(4-{4-(2,4-Dichloro-phenyl)-2-[2-(3′-trifluoromethyl-biphenyl-4-yl)-(E)-vinyl]-imidazol-1-ylmethyl}-phenyl) -acetic acid methyl ester (310 mg, 0.5 mmol) was hydrolyzed according to General Procedure F to give (4-{4-(2,4-Dichloro-phenyl)-2-[2-(3′-trifluoromethyl-biphenyl-4-yl)-(E)-vinyl]-imidazol-1-ylmethyl}-phenyl) -acetic acid (198 mg, 65%).

LCMS: 607 (M+H)^{+ 1}H NMR (DMSO, 400 MHz): δ 3.81 (s, 2H), 5.56 (s, 2H), 7.44-7.48 (m, 2H), 7.50-7.53 (m, 2H), 7.58 (s, 1H), 7.61-7.64 (m, 2H), 7.75-7.76 (m, 2H), 7.79-7.82 (m, 2H), 7.83-8.07 (m, 4H), 8.09 (d, 1H), 8.19 (s, 1H), 8.27 (d, 1H) ppm.

### Example 76

Trans 5-bromo 2-methoxy cinnamic acid (257 mg, 1 mmol) was reacted with 2-bromo-2,4-dichloro acetophenone (267 mg, 1 mml) according to general procedure A and obtained 2-[2-(5-Bromo-2-methoxy-phenyl)-(E)-vinyl]-4-(2,4-dichloro-phenyl)-1H-imidazole (424 mg, 1 mmol) was N-alkylated with methyl-4-(bromomethyl)benzoate (229 mg, 1 mmol) following general procedure E. The resulted 4-[2-[2-(5-Bromo-2-methoxy-phenyl)-(E)-vinyl]-4-(2,4-dichloro-phenyl)-imidazol-1-ylmethyl]-benzoic acid methyl ester (572 mg, 1 mmol) was coupled with 4-hydroxy-phenyl boronic acid (137 mg, 1 mmol) following General Procedure B to give 4-{4-(2,4-Dichloro-phenyl)-2-[2-(4′-hydroxy-4-methoxy-biphenyl-3-yl)-(E)-vinyl]-imidazol-1-ylmethyl}-benzoic acid methyl ester (291 mg, 50%).

LCMS: 585 (M+H)⁺.

### Example 77

4-{4-(2,4-Dichloro-phenyl)-2-[2-(4′-hydroxy-4-methoxy-biphenyl-3-yl)-(E)-vinyl]-imidazol-1-ylmethyl}-benzoic acid methyl ester (146 mg, 0.25 mmol) was hydrolyzed according to General Procedure F to give 4-{4-(2,4-Dichloro-phenyl)-2-[2-(4′-hydroxy-4-methoxy-biphenyl-3-yl)-(E)-vinyl]-imidazol-1-ylmethyl}-benzoic acid (107 mg, 75%).

LCMS: 571 (M+H)⁺. ¹H NMR (DMSO, 400 MHz): 6, 3.87 (s, 3H), 5.26 (d, 2H), 7.13 (d, 2H), 7.16-7.22 (m, 2H), 7.28-7.36 (m, 2H), 7.39 (d, 1H), 7.41-7.56 (m, 4H), 7.70 (d, 1H), 7.76-8.11 (m. 4H), 8.14 (d, 1H) ppm

### Example 78

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-butoxy-phenyl boronic acid (195 mg, 1 mmol) following General Procedure B to give 4-2-[2-(3′-butoxy-biphenyl-4-yl)-(E)-vinyl]-4-{4-(2,4-dichloro-phenyl)-imidazol-1yl-methyl} benzoic acid methyl ester (325 mg, 53%).

LCMS: 611 (M+H)⁺

### Example 79

4-2-[2-(3′-butoxy-biphenyl-4-yl)-(E)-vinyl]-4-{4-(2,4-dichloro-phenyl)-imidazol-1-yl-methyl} benzoic acid methyl ester (305 mg, 0.5 mmol) was hydrolyzed according to General Procedure F to give 4-[2-[2-(3′-Butoxy-biphenyl-4-yl)-(E)-vinyl]-4-(2,4-dichloro-phenyl)-imidazol-1-ylmethyl]-benzoic acid (192 mg, 64%)

LCMS: 597 (M+H)^{+ 1}H NMR (DMSO, 400 MHz): δ 0.94 (t, 3H), 1.41-1.44 (m, 2H), 1.68-1.72 (m, 2H), 4.01 (q, 2H), 5.66 (s, 2H), 7.10 (d, 1H), 7.29 (s, 1H), 7.31-7.36 (m, 4H), 7.51-7.56 (m, 4H), 7.59-7.66 (m, 4H), 7.67 (d, 1H), 7.91 (d, 1H), 8.11 (s, 1H), 8.29 (d, 1H) ppm.

### Example 80

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-3-(bromomethyl)benzoate (229 mg, 1 mmol) following general procedure E. The resulted 3-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-butoxy-phenyl boronic acid (195 mg, 1 mmol) following General Procedure B to give 3-2-[2-(4′-butoxy-biphenyl-4-yl)-(E)-vinyl]-4-{4-(2,4-dichloro-phenyl)-imidazol-1yl-methyl} benzoic acid methyl ester (319 mg, 52%).

LCMS: 611 (M+H)⁺

### Example 81

3-2-[2-(4′-butoxy-biphenyl-4-yl)-(E)-vinyl]-4-{4-(2,4-dichloro-phenyl)-imidazol-1-yl-methyl} benzoic acid methyl ester (305 mg, 0.5 mmol) was hydrolyzed according to General Procedure F to give 3-[2-[2-(4′-Butoxy-biphenyl-4-yl)-(E)-vinyl]-4-(2,4-dichloro-phenyl)-imidazol-1-ylmethyl]-benzoic acid (191 mg, 64%)

LCMS: 597 (M+H)^{+ 1}H NMR (DMSO, 400 MHz): δ 0.97 (t, 3H), 1.42-1.46 (m, 2H), 1.69-1.71 (m, 2H), 4.01 (q, 2H), 5.67 (s, 2H), 7.04 (d, 1H), 7.27 (s, 1H), 7.34-7.38 (m, 4H), 7.51-7.55 (m, 4H), 7.57-7.63 (m, 4H), 7.64 (d, 1H), 7.90 (d, 1H), 8.09 (s, 1H), 8.21 (d, 1H) ppm.

### Example 82

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(methanesulfonyl)-phenyl boronic acid (200 mg, 1 mmol) following General Procedure B to give 4-2-[2-(4′-methanesulfonyl -biphenyl-4-yl)-(E)-vinyl]-4-{4-(2,4-dichloro-phenyl)-imidazol-1yl-methyl} benzoic acid methyl ester (294 mg, 47%)

LCMS: 617 (M+H)⁺.

### Example 83

4-{4-(2,4-Dichloro-phenyl)-2-[2-(4′-methanesulfonyl-biphenyl-4-yl)-(E)-vinyl]-imidazol-1-ylmethyl}-benzoic acid methyl ester (155 mg, 0.25 mmol) was hydrolyzed according to General Procedure F to give 4-{4-(2,4-Dichloro-phenyl)-2-[2-(4′-methanesulfonyl-biphenyl-4-yl)-(E)-vinyl]-imidazol-1-ylmethyl}-benzoic acid (108 mg, 72%)

LCMS: 603 (M+H)^{+ 1}H NMR (DMSO, 400 MHz): δ 3.47 (s, 3H), 5.66 (s, 2H), 7.12 (d, 1H), 7.36 (s, 1H), 7.37-7.40 (m, 4H), 7.52-7.54 (m, 4H), 7.58-7.64 (m, 4H), 7.66 (d, 1H), 7.91 (d, 1H), 8.09 (s, 1H), 8.21 (d, 1H) ppm.

### Example 84

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methanesulfonyl)-phenyl boronic acid (200 mg, 1 mmol) following General Procedure B to give 4-2-[2-(3′-methanesulfonyl -biphenyl-4-yl)-(E)-vinyl]-4-{4-(2,4-dichloro-phenyl)-imidazol-1yl-methyl} benzoic acid methyl ester (299 mg, 48%)

LCMS: 617 (M+H)⁺.

### Example 85

4-{4-(2,4-Dichloro-phenyl)-2-[2-(3′-methanesulfonyl-biphenyl-4-yl)-(E)-vinyl]-imidazol-1-ylmethyl}-benzoic acid methyl ester (155 mg, 0.25 mmol) was hydrolyzed according to General Procedure F to give 4-{4-(2,4-Dichloro-phenyl)-2-[2-(3′-methanesulfonyl-biphenyl-4-yl)-(E)-vinyl]-imidazol-1-ylmethyl}-benzoic acid (101 mg, 67%)

LCMS: 603 (M+H)^{+ 1}H NMR (DMSO, 400 MHz): δ 3.31 (s, 3H), 5.51 (s, 2H), 7.23 (s, 1H), 7.45-7.49 (m, 2H), 7.51-7.57 (m, 2H), 7.61-7.64 (m, 2H), 7.75-7.76 (m, 2H), 7.79-7.82 (m, 2H), 7.84-8.07 (m, 4H), 8.10 (d, 1H), 8.19 (s, 1H), 8.25 (d, 1H) ppm.

### Example 86

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 1-(tert-butoxycarbonyl)-pyrrole-2-boronic acid (211 mg, 1 mmol) following General Procedure B to give 2-(4-{2-[4-(2,4-Dichloro-phenyl)-1-(4-methoxycarbonyl-benzyl)-1H-imidazol-2-yl]-(E)-vinyl}-phenyl)-pyrrole-1-carboxylic acid tert-butyl ester (278 mg, 44%)

LCMS: 628 (M+H)⁺.

### Example 87

2-(4-{2-[4-(2,4-Dichloro-phenyl)-1-(4-methoxycarbonyl-benzyl)-1H-imidazol-2-yl]-(E)-vinyl}-phenyl)-pyrrole-1-carboxylic acid tert-butyl ester (157 mg, 0.25 mmol) was hydrolyzed according to General Procedure F to give 2-(4-{2-[1-(4-Carboxy-benzyl)-4-(2,4-dichloro-phenyl)-1H-imidazol-2-yl]-(E)-vinyl}-phenyl)-pyrrole-1-carboxylic acid tert-butyl ester (89 mg, 59%)

LCMS: 614 (M+H)⁺.

### Example 88

2-(4-{2-[1-(4-Carboxy-benzyl)-4-(2,4-dichloro-phenyl)-1H-imidazol-2-yl]-(E)-vinyl}-phenyl)-pyrrole-1-carboxylic acid tert-butyl ester (62 mg, 0.1 mmol) was de-protected according to General Procedure O to give 4-(4-(2,4-Dichloro-phenyl)-2-{2-[4-(1H-pyrrol-2-yl)-phenyl]-(E)-vinyl}-imidazol-1-ylmethyl)-benzoic acid (29 mg, 55%).

LCMS: 514 (M+H)⁺.

### Example 89

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B and obtained 4-2-[2-(4′-hydroxy-biphenyl-4-yl)-(E)-vinyl]-4-{4-(2,4-dichloro-phenyl)-imidazol-1yl-methyl} benzoic acid methyl ester (278 mg, 0.5 mmol) was alkylated with 4-fluoronitro benzene (71 mg, 0.5 mmol) according to general procedure I to give 4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (221 mg, 65%).

LCMS: 676 (M+H)⁺.

### Example 90

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (169 mg, 0.25 mmol) was hydrolyzed according to General Procedure F to give 4-[2-{2-[4′-(4-Nitro-phenoxy)-biphenyl-4-yl]-(E)-vinyl}-4-(2,4-dichloro-phenyl)-imidazol-1-ylmethyl]-benzoic acid (125 mg, 75%).

LCMS: 662 (M+H)⁺.

### Example 91

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (169 mg, 0.25 mmol) was reduced according to general procedure K to give 4-[2-{2-[4′-(4-amino-phenoxy)-biphenyl-4-yl]-(E)-vinyl}-4-(2,4-dichloro-phenyl)-imidazol-1-ylmethyl]-benzoic acid methyl ester (112 mg, 69%).

LCMS: 646 (M+H)⁺.

### Example 92

4-[2-{2-[4′-(4-amino-phenoxy)-biphenyl-4-yl]-(E)-vinyl}-4-(2,4-dichloro-phenyl)-imidazol-1-ylmethyl]-benzoic acid methyl ester (65 mg, 0.1 mmol) was coupled with methanesulfonyl chloride (12 mg, 0.1 mmol) following general procedure L to give 4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid methyl ester (41 mg, 57%).

LCMS: 724 (M+H)⁺.

### Example 93

4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid methyl ester (36 mg, 0.05 mmol) was hydrolyzed according to General Procedure F to give 4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid (20 mg, 64%).

LCMS: 710 (M+H)⁺

### Example 94

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methanesulfonylamino)-phenyl boronic acid (215 mg, 1 mmol) following General Procedure B to give 4-2-[2-(3′-methanesulfonylamino -biphenyl-4-yl)-(E)-vinyl]-4-{4-(2,4-dichloro-phenyl)-imidazol-1yl-methyl} benzoic acid methyl ester (304 mg, 48%)

LCMS: 632 (M+H)⁺.

### Example 95

4-{4-(2,4-Dichloro-phenyl)-2-[2-(3′-methanesulfonylamino -biphenyl-4-yl)-(E)-vinyl]-imidazol-1-ylmethyl}-benzoic acid methyl ester (158 mg, 0.25 mmol) was hydrolyzed according to General Procedure F to give 4-{4-(2,4-Dichloro-phenyl)-2-[2-(3′-methane-sulfonylamino -biphenyl-4-yl)-(E)-vinyl]-imidazol-1-ylmethyl}-benzoic acid (109 mg, 70%)

LCMS: 618 (M+H)⁺; ¹H NMR (DMSO, 400 MHz): δ 3.38 (s, 3H), 5.64 (s, 2H), 7.21 (d, 1H), 7.33-7.42 (m, 4H), 7.43-7.52 (m, 4H), 7.56-7.75 (m, 4H). 7.77 (d, 1H), 7.92 (d, 1H), 8:11 (s, 1H), 8327 (d, 1H), 9.85 (s, 1H), 13.02 (s, 1H) ppm.

### Example 96

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(methanesulfonylamino)-phenyl boronic acid (215 mg, 1 mmol) following General Procedure B to give 4-2-[2-(4′-methanesulfonylamino -biphenyl-4-yl)-(E)-vinyl]-4-{4-(2,4-dichloro-phenyl)-imidazol-1yl-methyl} benzoic acid methyl ester (308 mg, 48%)

LCMS: 632 (M+H)⁺

### Example 97

4-{4-(2,4-Dichloro-phenyl)-2-[2-(4′-methanesulfonylamino -biphenyl-4-yl)-(E)-vinyl]-imidazol-1-ylmethyl}-benzoic acid methyl ester (158 mg, 0.25 mmol) was hydrolyzed according to General Procedure F to give 4-{4-(2,4-Dichloro-phenyl)-2-[2-(4′-methanesulfonylamino -biphenyl-4-yl)-(E)-vinyl]imidazol-1-ylmethyl}-benzoic acid (101 mg, 66%)

LCMS: 618 (M+H)^{+ 1}H NMR (DMSO, 400 MHz): δ 3.47 (s, 3H). 5.64 (s, 2H), 6.70 (d, 2H), 7.01 (d, 2H), 7.28-7.30 (m, 2H). 7.35-7.37 (m, 2H). 7.51-7.59 (m, 2H), 7.65-712 (m, 2H), 7.74 (d, 1H), 7.93 (s, 1H), 8.11 (s, 1H), 8.27 (d. 1H), 9.18 (s, 1H), 9.37 (s, 1H), 13.01 (s, 1H) ppm.

### Example 98

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methoxycarbonyl)-phenyl boronic acid (179 mg, 1 mmol) following General Procedure B to give 4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester (289 mg, 48%)

LCMS: 597 (M+H)⁺.

### Example 99

4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester (149 mg, 0.25 mmol) was hydrolyzed according to General Procedure F to give 4′-{2-[4-(2,4-Dichloro-phenyl)-1-(4-methoxycarbonyl-benzyl)-1H-imidazol-2-yl]-(E)-vinyl}-biphenyl-3-carboxylic acid (99 mg, 69%)

LCMS: 569 (M+H)⁺; ¹H NMR (DMSO, 400 MHz): δ 5.70 (s, 2H), 7.39-7.45 (m, 4H), 7.54 (d, 1H), 7.61 (d. 1H), 7.70-7.74 (m, 4H), 7.76 (d, 1H), 7.79-7.96 (m, 4H), T98 (s, 1H), 8.17 (d, 1H), 8.22 (d, 1H) ppm.

### Example 100

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B and obtained 4-2-[2-(4′-hydroxy-biphenyl-4-yl)-(E)-vinyl]-4-{4-(2,4-dichloro-phenyl)-imidazol-1yl-methyl}benzoic acid methyl ester (277 mg, 0.5 mmol) was alkylated with 1-bromo-4,4,4-trifluorobutane following general procedure E to give 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid methyl ester (214 mg, 64%).

LCMS: 665 (M+H)⁺.

### Example 101

4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid methyl ester (166 mg, 0.25 mmol) was hydrolyzed according to General Procedure F to give 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid (106 mg, 65%)

LCMS: 651 (M+H)^{+ 1}H NMR (DMSO, 400 MHz): δ 1.41-1.44 (m, 2H), 1.66-1.71 (m, 2H), 2.41-2.47 (m, 2H), 5.66 (s, 2H), 7.12 (d, 1H), 7.19 (s, 1H), 7.33-7.37 (m, 4H), 7.51-7.55 (m, 4H), 7.56-7.62 (m, 4H), 7.65 (d, 1H), 7.91 (d, 1H), 8.11 (s, 1H), 8.29 (d, 1H) ppm.

### Example 102

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 2-methoxy-5-pyridine boronic acid (153 mg, 1 mmol) following General Procedure B to give 4-(4-(2,4-Dichloro-phenyl)-2-{2-[4-(6-methoxy-pyridin-3-yl)-phenyl]-(E)-vinyl}-imidazol-1-ylmethyl)-benzoic acid methyl ester (289 mg, 50%)

LCMS: 570 (M+H)⁺

### Example 103

4-(4-(2,4-Dichloro-phenyl)-2-{2-[4-(6-methoxy-pyridin-3-yl)-phenyl]-(E)-vinyl}-imidazol-1-ylmethyl)-benzoic acid methyl ester (143 mg, 0.25 mmol) was hydrolyzed according to General Procedure F to give 4-(4-(2,4-Dichloro-phenyl)-2-{2-[4-(6-methoxy-pyridin-3-yl)-phenyl]-(E)-vinyl}-imidazol-1-ylmethyl)-benzoic acid (95 mg, 68%)

LCMS: 556 (M+H)^{+ 1}H NMR (DMSO, 400 MHz): δ 3.79 (s, 3H), 5.68 (s, 2H), 7.01 (d, 1H), 7.26 (s, 1H), 7.36-7.40 (m, 3H), 7.51-7.56 (m, 3H), 7.58-7.64 (m, 4H), 7.67 (d, 1H), 7.92 (d, 1H), 8.11 (s, 1H), 8.27 (d, 1H) ppm.

### Example 104

4-Hydroxy-4-biphenyl carboxylic acid (214 mg, 1 mmol) was reacted with 2-bromo-2,4-dichloro acetophenone (267 mg, 1 mmol) according to general procedure A and obtained 4′-[4-(2,4-Dichloro-phenyl)-1H-imidazol-2-yl]-biphenyl-4-ol (381 mg, 1 mmol) was N-alkylated with methyl-4-(bromomethyl)benzoate (229 mg, 1 mmol) following general procedure E to give 4-[4-(2,4-Dichloro-phenyl)-2-(4′-hydroxy-biphenyl-4-yl)-imidazol-1-ylmethyl]-benzoic acid methyl ester (312 mg, 59%).

LCMS: 529 (M+H)⁺.

### Example 105

4-[4-(2,4-Dichloro-phenyl)-2-(4′-hydroxy-biphenyl-4-yl)-imidazol-1-ylmethyl]-benzoic acid methyl ester (264 mg, 0.5 mmol) was hydrolyzed according to General Procedure F to give 4-[4-(2,4-Dichloro-phenyl)-2-(4′-hydroxy-biphenyl-4-yl)-imidazol-1-ylmethyl]-benzoic acid (186 mg, 72%).

LCMS: 515 (M+H)^{+ 1}H NMR (DMSO, 400 MHz): δ 5.54 (s, 2H), 6.81-6.86 (m, 5H), 7.23 (d, 1H), 7.41-7.57 (m, 5H), 7.74 (d, 1H), 7.89 (d, 1H), 7.94 (d, 1H), 8.11 (s, 1H), 8.27 (d, 1H) ppm.

### Example 106

4-Hydroxy-4-biphenyl carboxylic acid (214 mg, 1 mmol) was reacted with 2-bromo-2,4-dichloro acetophenone (267 mg, 1 mmol) according to general procedure A and obtained 4′-[4-(2,4-Dichloro-phenyl)-1H-imidazol-2-yl]-biphenyl-4-ol (381 mg, 1 mmol) was N-alkylated with methyl-4-(bromomethyl)benzoate (229 mg, 1 mmol) following general procedure E. The resulted 4-[4-(2,4-Dichloro-phenyl)-2-(4′-hydroxy-biphenyl-4-yl)-imidazol-1-ylmethyl]-benzoic acid methyl ester (265 mg, 0.5 mmol) was alkylated with bromo ethane (55 mg, 0.5 mmol) following general procedure E to give 4-[4-(2,4-Dichloro-phenyl)-2-(4′-ethoxy-biphenyl-4-yl)-imidazol-1-ylmethyl]-benzoic acid methyl ester (191 mg, 68%).

LCMS: 557 (M+H)⁺.

### Example 107

4-[4-(2,4-Dichloro-phenyl)-2-(4′-ethoxy-biphenyl-4-yl)-imidazol-1-ylmethyl]-benzoic acid methyl ester (278 mg, 0.5 mmol) was hydrolyzed according to General Procedure F to give 4-[4-(2,4-Dichloro-phenyl)-2-(4′-ethoxy-biphenyl-4-yl)-imidazol-1-ylmethyl]-benzoic acid (189 mg, 69%).

LCMS: 543 (M+H)⁺; ¹H NMR (DMSO, 400 MHz): δ 0.94 (t, 3H), 4.07 (q, 2H), 5.56 (s, 2H), 6.83-6.88 (m, 4H), 7.21 (d, 1H), 7.43-7.58 (m, 4H), 7.65-7.69 (m, 2H), 7.71 (d, 1H), 7.90 (d, 1H), 7.94 (d, 1H), 8.12 (s, 1H), 8.28 (d, 1H) ppm.

### Example 108

4-Bromo benzoic acid (201 mg, 1 mmol) was reacted with 2-bromo-2,4-dichloro acetophenone (267 mg, 1 mmol) according to general procedure A and obtained 2-(4-bromo-phenyl)-4-(2,4-dichloro-phenyl)-1H-imidazole (368 mg, 1 mmol) was N-alkylated with methyl-4-(bromomethyl)benzoate (229 mg, 1 mmol) following general procedure E. The resulted 4-[2-(4-Bromo-phenyl)-4-(2,4-dichloro-phenyl)-imidazol-1-ylmethyl]-benzoic acid methylester (516 mg, 1 mmol) was coupled with 3-(methanesulfonyl)-phenyl boronic acid (200 mg, 1 mmol) following General Procedure B to give 4-[4-(2,4-Dichloro-phenyl)-2-(3′-methanesulfonyl-biphenyl-4-yl)-imidazol-1-ylmethyl]-benzoic acid methyl ester (324 mg, 55%).

LCMS: 591 (M+H)⁺

### Example 109

4-[4-(2,4-Dichloro-phenyl)-2-(3′-methanesulfonyl-biphenyl-4-yl)-imidazol-1-ylmethyl]-benzoic acid methyl ester (295 mg, 0.5 mmol) was hydrolyzed according to General Procedure F to give 4-[4-(2,4-Dichloro-phenyl)-2-(3′-methanesulfonyl-biphenyl-4-yl)-imidazol-1-ylmethyl]-benzoic acid (201 mg, 69%).

LCMS: 577 (M+H)^{+ 1}H NMR (DMSO, 400 MHz): δ 3.31 (s, 3H), 5.64 (s, 2H), 7.25-7.33 (m, 4H), 7.60 (d, 1H), 7.76 (s, 1H), 7.82 (d, 1H), 7.84 (d, 1H), 7.90-7.96 (m, 4H), 8.10 (d, 1H), 8.18 (d, 1H), 8.23 (s, 1H), 8.30 (s, 1H) ppm.

### Example 110

4-{4-(2,4-dichloro-phenyl)-2-[2-(4′-trifluoromethyl-biphenyl-4-yl)-(E)-vinyl]-imidazol-1yl-methyl} benzoic acid (148 mg, 0.25 mmol) was reduced according to General Procedure V to give 4-{4-(2,4-Dichloro-phenyl)-2-[2-(4′-trifluoromethyl-biphenyl-4-yl)-ethyl]-imidazol-1-ylmethyl}-benzoic acid (79 mg, 53%).

LCMS: 595 (M+H)^{+ 1}H NMR (DMSO, 400 MHz): δ 2.92-2.94 (m, 2H), 2.98-3.0 (m, 2H), 5.64 (d, 2H), 7.20 (d, 1H), 7.31-7.38 (m, 2H), 7.42-7.52 (m, 2H), 7.58-7.65 (m, 2H), 7.75-7.79 (m, 2H), 7.80-7.95 (m, 4H), 8.11 (s, 1H), 8.22 (d, 1H), 8.30 (d, 1H) ppm.

### Example 111

4-Bromophenylacetic acid (107.5 g, 0.5 mol) was treated according to general procedure A using 2,4-dichlorophenacyl bromide to give 2-(4-bromo-benzyl)-4-(2,4-dichloro-phenyl)-1H-imidazole (38.2 g, 20%). LCMS: m/z 382 (M+H)⁺;

2-(4-Bromo-benzyl)-4-(2,4-dichloro-phenyl)-1H-imidazole (19.1 g, 50 mmol) was treated as described in general procedure E using methyl 4-(bromomethyl)benzoate to give 4-[2-(4-bromo-benzyl)-4-(2,4-dichloro-phenyl)-imidazol-1-ylmethyl]-benzoic acid methyl ester (17.5 g, 66%). LCMS: m/z 530 (M+H)⁺;

4-[2-(4-Bromo-benzyl)-4-(2,4-dichloro-phenyl)-imidazol-1-ylmethyl]-benzoic acid methyl ester (106 mg, 0.2 mmol) was treated as described in general procedure B using 2-methoxyphenylboronic acid (46 mg, 0.3 mmol) to give 4-[4-(2,4-dichloro-phenyl)-2-(2′-methoxy-biphenyl-4-ylmethyl)-imidazol-1-ylmethyl]-benzoic acid methyl ester (75 mg, 67%). LCMS: m/z 557 (M+H)⁺.

4-[4-(2,4-Dichloro-phenyl)-2-(2′-methoxy-biphenyl-4-ylmethyl)-imidazol-1-ylmethyl]-benzoic acid (48 mg, 88%) is prepared according to general procedure F using 4-[4-(2,4-dichloro-phenyl)-2-(2′-methoxy-biphenyl-4-ylmethyl)-imidazol-1-ylmethyl]-benzoic acid methyl ester (56 mg, 0.1 mmol).

LCMS: m/z 543 (M+H)⁺; ¹H NMR (DMSO-d₆, 400 MHz): δ 3.79 (s, 3H), 4.12 (s, 2H), 5.35 (s, 2H), 7.13 (d, 2H), 7.25 (d, 2H), 7.39 (d, 2H), 7.43 (dd, 1H), 7.68 (d, 2H), 7.74 (d, 2H), 7.80-7.97 (m, 4H), 8.06 (d, 1H) ppm.

### Example 112

4-[2-(4-Bromo-benzyl)-4-(2,4-dichloro-phenyl)-imidazol-1-ylmethyl]-benzoic acid methyl ester (106 mg, 0.2 mmol) was treated as described in general procedure B using [(3-methylsulfonyl)aminophenyl]boronic acid (64 mg, 0.3 mmol) to give 4-[4-(2,4-dichloro-phenyl)-2-(3′-methanesulfonylamino-biphenyl-4-ylmethyl)-imidazol-1-ylmethyl]-benzoic acid methyl ester (83 mg, 67%).

LCMS: m/z 620 (M+H)⁺.

### Example 113

4-[4-(2,4-Dichloro-phenyl)-2-(3′-methanesulfonylamino-biphenyl-4-ylmethyl)-imidazol-1-ylmethyl]-benzoic acid (56 mg, 92%) is prepared according to general procedure F using 4-[4-(2,4-dichloro-phenyl)-2-(3′-methanesulfonylamino-biphenyl-4-ylmethyl)-imidazol-1-ylmethyl]-benzoic acid methyl ester (62 mg, 0.1 mmol).

LCMS: m/z 606 (M+H)⁺; ¹H NMR (DMSO-d₆, 400 MHz): δ 3.27 (s, 3H), 4.14 (s, 2H), 5.36 (s, 2H), 7.15 (d, 2H), 7.26 (d, 2H), 7.39 (d, 2H), 7.43 (dd, 1H), 7.68 (d, 2H), 7.74 (d, 2H), 7.81-7.99 (m, 4H), 8.16 (d, 1H) ppm.

### Example 114

4-[2-(4-Bromo-benzyl)-4-(2,4-dichloro-phenyl)-imidazol-1-ylmethyl]-benzoic acid methyl ester (106 mg, 0.2 mmol) was treated as described in general procedure B using [(4-methylsulfonyl)aminophenyl]boronic acid (64 mg, 0.3 mmol) to give 4-[4-(2,4-dichloro-phenyl)-2-(4′-methanesulfonylamino-biphenyl-4-ylmethyl)-imidazol-1-ylmethyl]-benzoic acid methyl ester (77 mg, 62%).

LCMS: m/z 620 (M+H)⁺.

### Example 115

4-[4-(2,4-Dichloro-phenyl)-2-(4′-methanesulfonylamino-biphenyl-4-ylmethyl)-imidazol-1-ylmethyl]-benzoic acid (51 mg, 84%) is prepared according to general procedure F using 4-[4-(2,4-dichloro-phenyl)-2-(4′-methanesulfonylamino-biphenyl-4-ylmethyl)-imidazol-1-ylmethyl]-benzoic acid methyl ester (62 mg, 0.1 mmol).

LCMS: m/z 606 (M+H)⁺; ¹H NMR (DMSO-d₆, 400 MHz): δ 3.28 (s, 3H), 4.13 (s, 2H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.14 (d, 1H) ppm.

### Example 116

4-[2-(4-Bromo-benzyl)-4-(2,4-dichloro-phenyl)-imidazol-1-ylmethyl]-benzoic acid methyl ester (1.06 g, 2 mmol) was treated as described in general procedure B using 3-aminobenzeneboronic acid (548 mg, 4 mmol) to give 4-[2-(3′-amino-biphenyl-4-ylmethyl)-4-(2,4-dichloro-phenyl)-imidazol-1-ylmethyl]-benzoic acid methyl ester (531 mg, 49%). LCMS: m/z 542 (M+H)⁺.

4-[2-(3′-Amino-biphenyl-4-ylmethyl)-4-(2,4-dichloro-phenyl)-imidazol-1-ylmethyl]-benzoic acid methyl ester (54 mg, 0.1 mmol) was treated as described in general procedure L using trifluoromethanesulfonic anhydride (21 μL, 0.12 mmol) to give 4-[4-(2,4-dichloro-phenyl)-2-(3′-trifluoromethanesulfonylamino-biphenyl-4-ylmethyl)-imidazol-1-ylmethyl]-benzoic acid methyl ester (55 mg, 82%). LCMS: m/z 674 (M+H)⁺.

### Example 117

4-[4-(2,4-Dichloro-phenyl)-2-(3′-trifluoromethanesulfonylamino-biphenyl-4-ylmethyl)-imidazol-1-ylmethyl]-benzoic acid (14 mg, 42%) is prepared according to general procedure F using 4-[4-(2,4-dichloro-phenyl)-2-(3′-trifluoromethanesulfonyl-amino-biphenyl-4-ylmethyl)-imidazol-1-ylmethyl]-benzoic acid methyl ester (34 mg, 0.05 mmol).

LCMS: m/z 660 (M+H)⁺; ¹H NMR (DMSO-d₆, 400 MHz): δ 4.14 (s, 2H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.16 (d, 1H) ppm.

### Example 118

4-[2-(3′-Amino-biphenyl-4-ylmethyl)-4-(2,4-dichloro-phenyl)-imidazol-1-ylmethyl]-benzoic acid methyl ester (54 mg, 0.1 mmol) was treated as described in general procedure L using ethanesulfonyl chloride (12 μL, 0.12 mmol) to give 4-[4-(2,4-dichloro-phenyl)-2-(3′-ethanesulfonylamino-biphenyl-4-ylmethyl)-imidazol-1-ylmethyl]-benzoic acid methyl ester (48 mg, 75%). LCMS: m/z 634 (M+H)⁺.

### Example 119

4-[4-(2,4-Dichloro-phenyl)-2-(3′-ethanesulfonylamino-biphenyl-4-ylmethyl)-imidazol-1-ylmethyl]-benzoic acid (15 mg, 48%) is prepared according to general procedure F using 4-[4-(2,4-dichloro-phenyl)-2-(3′-ethanesulfonylamino-biphenyl-4-ylmethyl)-imidazol-1-ylmethyl]-benzoic acid methyl ester (32 mg, 0.05 mmol).

LCMS: m/z 620 (M+H)⁺; ¹H NMR (DMSO-d₆, 400 MHz): δ 1.64 (t, 3H), 3.75 (q, 2H), 4.14 (s, 2H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.13 (d, 1H) ppm.

### Example 120

4-[2-(3′-Amino-biphenyl-4-ylmethyl)-4-(2,4-dichloro-phenyl)-imidazol-1-ylmethyl]-benzoic acid methyl ester (54 mg, 0.1 mmol) was treated as described in general procedure L using 1-propanesulfonyl chloride (14 μL, 0.12 mmol) to give 4-[4-(2,4-dichloro-phenyl)-2-(3′-propanesulfonylamino-biphenyl-4-ylmethyl)-imidazol-1-ylmethyl]-benzoic acid methyl ester (43 mg, 66%). LCMS: m/z 648 (M+H)⁺.

### Example 121

4-[4-(2,4-Dichloro-phenyl)-2-(3′-propanesulfonylamino-biphenyl-4-ylmethyl)-imidazol-1-ylmethyl]-benzoic acid (12 mg, 38%) is prepared according to general procedure F using 4-[4-(2,4-dichloro-phenyl)-2-(3′-propanesulfonylamino-biphenyl-4-ylmethyl)-imidazol-1-ylmethyl]-benzoic acid methyl ester (32 mg, 0.05 mmol).

LCMS: m/z 634 (M+H)⁺; ¹H NMR (DMSO-d₆, 400 MHz): δ 1.26 (t, 3H), 2.13 (m, 2H), 3.65 (t, 2H), 4.14 (s, 2H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.14 (d, 1H) ppm.

### Example 122

4-[2-(3′-Amino-biphenyl-4-ylmethyl)-4-(2,4-dichloro-phenyl)-imidazol-1-ylmethyl]-benzoic acid methyl ester (54 mg, 0.1 mmol) was treated as described in general procedure L using methyl chloroformate (10 μL, 0.12 mmol) to give 4-[4-(2,4-dichloro-phenyl)-2-(3′-methoxycarbonylamino-biphenyl-4-ylmethyl)-imidazol-1-ylmethyl]-benzoic acid methyl ester (50 mg, 83%). LCMS: m/z 600 (M+H)⁺.

### Example 123

4-[4-(2,4-Dichloro-phenyl)-2-(3′-methoxycarbonylamino-biphenyl-4-ylmethyl)-imidazol-1-ylmethyl]-benzoic acid (20 mg, 68%) is prepared according to general procedure F using 4-[4-(2,4-dichloro-phenyl)-2-(3′-methoxycarbonylamino-biphenyl-4-ylmethyl)-imidazol-1-ylmethyl]-benzoic acid methyl ester (30 mg, 0.05 mmol).

LCMS: m/z 586 (M+H)⁺; ¹H NMR (DMSO-d₆, 400 MHz): δ 3.79 (s, 3H), 4.14 (s, 2H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.81-8.00 (m, 4H), 8.11 (d, 1H) ppm.

### Example 124

4-[2-(3′-Amino-biphenyl-4-ylmethyl)-4-(2,4-dichloro-phenyl)-imidazol-1-ylmethyl]-benzoic acid methyl ester (54 mg, 0.1 mmol) was treated as described in general procedure L using isopropyl chloroformate (1.0 M in toluene, 0.12 mL, 0.12 mmol) to give 4-[4-(2,4-dichloro-phenyl)-2-(3′-isopropoxycarbonylamino-biphenyl-4-ylmethyl)-imidazol-1-ylmethyl]-benzoic acid methyl ester (38 mg, 61%).

LCMS: m/z 628 (M+H)⁺.

### Example 125

4-[4-(2,4-Dichloro-phenyl)-2-(3′-isopropoxycarbonylamino-biphenyl-4-ylmethyl)-imidazol-1-ylmethyl]-benzoic acid (18 mg, 58%) is prepared according to general procedure F using the methyl ester of Example 124 (31 mg, 0.05 mmol).

LCMS: m/z 614 (M+H)⁺; ¹H NMR (DMSO-d₆, 400 MHz): δ 1.31 (d, 6H), 4.14 (s, 2H), 5.02 (m, 1H), 5.35 (s, 2H), 7.15 (d, 2H), 7.26 (d, 2H), 7.39 (d, 2H), 7.43 (dd, 1H), 7.68 (d, 2H), 7.74 (d, 2H), 7.81-8.02 (m, 4H), 8.10 (d, 1H) ppm.

By analagous methods to those used to prepare Example 125, the following compounds were synthesized:

| Example | Name | LC/MS (m/z) |
|---|---|---|
| 126 | 4-[4-(2,4-Dichloro-phenyl)-2- | 600 (M + H)⁺ |
| | (3′-ethoxycarbonylamino-biphenyl- | |
| | 4-ylmethyl)-imidazol-1-ylmethyl]-benzoic | |
| | acid | |
| 127 | 4-[4-(2,4-Dichloro-phenyl)-2- | 614 (M + H)⁺; |
| | (3′-propoxycarbonylamino-biphenyl- | |
| | 4-ylmethyl)-imidazol-1-ylmethyl]-benzoic | |
| | acid | |
| 128 | 4-[4-(2,4-Dichloro-phenyl)-2-(3′- | 628 (M + H)⁺ |
| | isobutoxycarbonylamino-biphenyl-4- | |
| | ylmethyl)-imidazol-1-ylmethyl]-benzoic | |
| | acid | |

### Example 129

4-[4-(2,4-Dichloro-phenyl)-2-(3′-methanesulfonyl-biphenyl-4-carbonyl)-imidazol-1-ylmethyl]-benzoic acid (7 mg, 12%) is prepared according to general procedure X using 4-[4-(2,4-dichloro-phenyl)-2-(3′-methanesulfonyl-biphenyl-4-ylmethyl)-imidazol-1-ylmethyl]-benzoic acid (59 mg, 0.1 mmol).

LCMS: m/z 605 (M+H)⁺; ¹H NMR (DMSO-d₆, 400 MHz): δ 3.28 (s, 3H), 5.39 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 130

4-[4-(2,4-Dichloro-phenyl)-2-(3′-trifluoromethyl-biphenyl-4-carbonyl)-imidazol-1-ylmethyl]-benzoic acid (8 mg, 14%) is prepared according to general procedure X using 4-[4-(2,4-dichloro-phenyl)-2-(3′-trifluoromethyl-biphenyl-4-ylmethyl)-imidazol-1-ylmethyl]-benzoic acid (58 mg, 0.1 mmol).

LCMS: m/z 595 (M+H)⁺; ¹H NMR (DMSO-d₆, 400 MHz): δ 5.40 (s, 2H), 7.16 (d, 2H), 7.29 (d, 2H), 7.43 (dd, 1H), 7.59 (d, 2H), 7.64 (d, 1H), 7.69-7.73 (m, 4H), 7.82 (d, 2H), 7.96 (s, 1H), 8.15 (d, 1H) ppm.

### Example 131

4-[4-(2,4-Dichloro-phenyl)-2-(3′-trifluoromethoxy-biphenyl-4-carbonyl)-imidazol-1-ylmethyl]-benzoic acid (9 mg, 15%) is prepared according to general procedure X using 4-[4-(2,4-dichloro-phenyl)-2-(3′-trifluoromethoxy-biphenyl-4-ylmethyl)-imidazol-1-ylmethyl]-benzoic acid (60 mg, 0.1 mmol).

LCMS: m/z 611 (M+H)⁺; ¹H NMR (DMSO-d₆, 400 MHz): δ 5.39 (s, 2H), 7.16 (d, 2H), 7.29 (d, 2H), 7.43 (dd, 1H), 7.59 (d, 2H), 7.64 (d, 1H), 7.69-7.73 (m, 4H), 7.82 (d, 2H), 7.96 (s, 1H), 8.14 (d, 1H) ppm.

### Example 132

### Step 1

1-(4-Methoxyphenyl)-1-cyclopropanecarboxylic acid (38.4 g, 0.2 mol) was treated according to general procedure A using 2,4-dichlorophenacyl bromide to give 4-(2,4-dichloro-phenyl)-2-[1-(4-methoxy-phenyl)-cyclopropyl]-1H-imidazole (23.0 g, 32%). The resulted 1H-imidazole intermediate (21.5 g, 60 mmol) was treated as described in general procedure E using methyl 4-(bromomethyl)benzoate to give 4-{4-(2,4-dichloro-phenyl)-2-[1-(4-methoxy-phenyl)-cyclopropyl]-imidazol-1-ylmethyl}-benzoic acid methyl ester (17.9 g, 59%). LCMS: m/z 507 (M+H)⁺4-{4-(2,4-Dichloro-phenyl)-2-[1-(4-hydroxy-phenyl)-cyclopropyl]-imidazol-1-ylmethyl}-benzoic acid methyl ester (9.9 g, 67%) was prepared according to general procedure C using 4-{4-(2,4-dichloro-phenyl)-2-[1-(4-methoxy-phenyl)-cyclopropyl]-imidazol-1-ylmethyl}-benzoic acid methyl ester (15.2 g, 30 mmol). LCMS: m/z 493 (M+H)⁺.

4-{4-(2,4-Dichloro-phenyl)-2-[1-(4-hydroxy-phenyl)-cyclopropyl]-imidazol-1-ylmethyl}-benzoic acid methyl ester (4.9 g, 10 mmol) was treated as described in general procedure W using 3-(trifluoromethyl)benzeneboronic acid (5.7 g, 30 mmol) to give 4-(4-(2,4-dichloro-phenyl)-2-{1-[4-(3-trifluoromethyl-phenoxy)-phenyl]-cyclopropyl}-imidazol-1-ylmethyl)-benzoic acid methyl ester (764 mg, 12%). LCMS: m/z 637 (M+H)⁺.

4-(4-(2,4-Dichloro-phenyl)-2-{1-[4-(3-trifluoromethyl-phenoxy)-phenyl]-cyclopropyl}-imidazol-1-ylmethyl)-benzoic acid (51 mg, 82%) is prepared according to general procedure F using 4-(4-(2,4-dichloro-phenyl)-2-{1-[4-(3-trifluoromethyl-phenoxy)-pheny]-cyclopropyl}-imidazol-1-ylmethyl)-benzoic acid methyl ester (64 mg, 0.1 mmol).

LCMS: m/z 623 (M+H)⁺; ¹H NMR (DMSO-d₆, 400 MHz): δ 1.16 (m, 2H), 1.42 (m, 2H), 5.36 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 133

4-(4-Iodo-phenyl)-butyric acid (29.0 g, 0.1 mol) was treated according to general procedure A using 2,4-dichlorophenacyl bromide to give 4-(2,4-dichloro-phenyl)-2-[3-(4-iodo-phenyl)-propyl]-1H-imidazole (15.5 g, 34%). The resulted 1H-imidazole intermediate (13.7 g, 30 mmol) was treated as described in general procedure E using methyl 4-(bromomethyl)benzoate to give 4-{4-(2,4-dichloro-phenyl)-2-[3-(4-iodo-phenyl)-propyl]-imidazol-1-ylmethyl}-benzoic acid methyl ester (11.1 g, 61%). LCMS: m/z 605 (M+H)⁺.

4-{4-(2,4-Dichloro-phenyl)-2-[3-(4-iodo-phenyl)-propyl]-imidazol-1-ylmethyl}-benzoic acid methyl ester (605 mg, 1 mmol) was treated as described in general procedure B using 3-(trifluoromethyl)benzeneboronic acid (228 mg, 1.2 mmol) to give 4-{4-(2,4-dichloro-phenyl)-2-[3-(3′-trifluoromethyl-biphenyl-4-yl)-propyl]-imidazol-1-ylmethyl}-benzoic acid methyl ester (243 mg, 39%). LCMS: m/z 623 (M+H)⁺.

4-{4-(2,4-Dichloro-phenyl)-2-[3-(3′-trifluoromethyl-biphenyl-4-yl)-propyl]-imidazol-1-ylmethyl}-benzoic acid (51 mg, 84%) is prepared according to general procedure F using 4-{4-(2,4-dichloro-phenyl)-2-[3-(3′-trifluoromethyl-biphenyl-4-yl)-propyl]-imidazol-1-ylmethyl}-benzoic acid methyl ester (62 mg, 0.1 mmol).

LCMS: m/z 609 (M+H)⁺; ¹H NMR (DMSO-d₆, 400 MHz): δ 2.02 (m, 2H), 2.68 (m, 4H), 5.34 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.16 (d, 1H) ppm.

### Example 134

4-{4-(2,4-Dichloro-phenyl)-2-[3-(4-iodo-phenyl)-propyl]-imidazol-1-ylmethyl}-benzoic acid methyl ester (605 mg, 1 mmol) was treated as described in general procedure B using (3-methanesulfonylphenyl)boronic acid (240 mg, 1.2 mmol) to give 4-{4-(2,4-dichloro-phenyl)-2-[3-(3′-methanesulfonyl-biphenyl-4-yl)-propyl]-imidazol-1-ylmethyl}-benzoic acid methyl ester (196 mg, 31%). LCMS: m/z 633 (M+H)⁺.

4-{4-(2,4-Dichloro-phenyl)-2-[3-(3′-methanesulfonyl-biphenyl-4-yl)-propyl]-imidazol-1-ylmethyl}-benzoic acid (47 mg, 76%) is prepared according to general procedure F using 4-{4-(2,4-dichloro-phenyl)-2-[3-(3′-methanesulfonyl-biphenyl-4-yl)-propyl]-imidazol-1-ylmethyl}-benzoic acid methyl ester (63 mg, 0.1 mmol).

LCMS: m/z 619 (M+H)⁺; ¹H NMR (DMSO-d₆, 400 MHz): δ 2.03 (m, 2H), 2.69 (m, 4H), 3.28 (s, 3H), 5.34 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 135

4-Bromophenoxyacetic acid (23.1 g, 0.1 mol) was treated according to general procedure A using 2,4-dichlorophenacyl bromide to give 2-(4-bromo-phenoxymethyl)-4-(2,4-dichloro-phenyl)-1H-imidazole (14.3 g, 36%). The resulted 1H-imidazole intermediate (11.9 g, 30 mmol) was treated as described in general procedure E using methyl 4-(bromomethyl)benzoate to give 4-[2-(4-bromo-phenoxymethyl)-4-(2,4-dichloro-phenyl)-imidazol-1-ylmethyl]-benzoic acid methyl ester (11.3 g, 69%). LCMS: m/z 546 (M+H)⁺

4-[2-(4-Bromo-phenoxymethyl)-4-(2,4-dichloro-phenyl)-imidazol-1-ylmethyl]-benzoic acid methyl ester (55 mg, 0.1 mmol) was treated as described in general procedure B using 4-(trifluoromethoxy)benzeneboronic acid (25 mg, 0.12 mmol) to give 4-[4-(2,4-dichloro-phenyl)-2-(4′-trifluoromethoxy-biphenyl-4-yloxymethyl)-imidazol-1-ylmethyl]-benzoic acid methyl ester (27 mg, 43%). LCMS: m/z 627 (M+H)⁺.

4-[4-(2,4-Dichloro-phenyl)-2-(4′-trifluoromethoxy-biphenyl-4-yloxymethyl)-imidazol-1-ylmethyl]-benzoic acid (15 mg, 84%) is prepared according to general procedure F using 4-[4-(2,4-dichloro-phenyl)-2-(4′-trifluoromethoxy-biphenyl-4-yloxymethyl)-imidazol-1-ylmethyl]-benzoic acid methyl ester (19 mg, 0.03 mmol).

LCMS: m/z 613 (M+H)⁺; ¹H NMR (DMSO-d₆, 400 MHz): δ 4.64 (s, 2H), 5.35 (s, 2H), 7.16 (d, 2H), 7.29 (d, 2H), 7.43 (dd, 1H), 7.59 (d, 2H), 7.64 (d, 1H), 7.69-7.73 (m, 4H), 7.82 (d, 2H), 7.96 (s, 1H), 8.14 (d, 1H) ppm.

By analagous methods to those used to prepare Example 135, the following compounds were synthesized:

| Example | Name | LC/MS (m/z) |
|---|---|---|
| 136 | 4-[4-(2,4-Dichloro-phenyl)-2-(3′- | 613 (M + H)⁺ |
| | trifluoromethoxy-biphenyl-4-yloxymethyl)- | |
| | imidazol-1-ylmethyl]-benzoic acid | |
| 137 | 4-[4-(2,4-Dichloro-phenyl)-2-(4′- | 559 (M + H)⁺ |
| | methoxy-biphenyl-4-yloxymethyl)-imidazol- | |
| | 1-ylmethyl]-benzoic acid | |
| 138 | 4-[4-(2,4-Dichloro-phenyl)-2-(2′,4′- | 589 (M + H)⁺ |
| | dimethoxy-biphenyl-4-yloxymethyl)- | |
| | imidazol-1-ylmethyl]-benzoic acid | |
| 139 | 4-[2-(4-Benzofuran-2-yl-phenoxymethyl)- | 569 (M + H)⁺ |
| | 4-(2,4-dichloro-phenyl)-imidazol-1- | |
| | ylmethyl]-benzoic acid | |
| 140 | 4-{4-(2,4-Dichloro-phenyl)-2-[4′-(propane- | 650 (M + H)⁺ |
| | 1-sulfonylamino)-biphenyl-4-yloxymethyl]- | |
| | imidazol-1-ylmethyl}-benzoic acid | |
| 141 | 4-{4-(2,4-Dichloro-phenyl)-2-[4′- | 699 (M + H)⁺ |
| | (4-methanesulfonyl-phenoxy)-biphenyl-4- | |
| | yloxymethyl]-imidazol-1-ylmethyl}- | |
| | benzoic acid | |

### Example 142

4-(4-Methoxy-phenyl)-butyric acid (2 g, 10 mmol) was treated according to general procedure A using 2,4-dichlorophenacyl bromide to give 4-(2,4-dichloro-phenyl)-2-[3-(4-methoxy-phenyl)-propyl]-1H-imidazole, which was treated as described in general procedure E using bromoethane to give the intermediate 4-(2,4-dichloro-phenyl)-1-ethyl-2-[3-(4-methoxy-phenyl)-propyl]-1H-imidazole, which was then treated as described in general procedure C to give 4-{3-[4-(2,4-dichloro-phenyl)-1-ethyl-1H-imidazol-2-yl]-propyl}-phenol (638 mg, 17%). LCMS: m/z 375 (M+H)⁺.

The phenol (375 mg, 1 mmol) was treated according to general procedure I using 5-fluoro-2-nitro-benzoic acid methyl ester to give 5-(4-{3-[4-(2,4-dichloro-phenyl)-1-ethyl-1H-imidazol-2-yl]-propyl}-phenoxy)-2-nitro-benzoic acid methyl ester, which was then treated as described in general procedure F to give 5-(4-{3-[4-(2,4-dichloro-phenyl)-1-ethyl-1H-imidazol-2-yl]-propyl}-phenoxy)-2-nitro-benzoic acid (308 mg, 57%).

LCMS: m/z 540 (M+H)⁺; ¹H NMR (DMSO-d₆, 400 MHz): δ 1.29 (t, 3H), 2.03 (m, 2H), 2.69 (m, 4H), 3.96 (q, 2H), 6.92 (d, 2H), 7.15 (d, 1H), 7.24 (d, 2H), 7.41 (dd, 2H), 7.57 (d, 2H), 7.78 (s, 1H), 8.15 (d, 1H) ppm.

### Example 143

The methyl ester of Example 142 (277 mg, 0.5 mmol) was treated according to general procedure K to give 2-amino-5-(4-{3-[4-(2,4-dichloro-phenyl)-1-ethyl-1H-imidazol-2-yl]-propyl}-phenoxy)-benzoic acid methyl ester, which was then treated as described in general procedure F to give 2-amino-5-(4-{3-[4-(2,4-dichloro-phenyl)-1-ethyl-1H-imidazol-2-yl]-propyl}-phenoxy)-benzoic acid (120 mg, 47%).

LCMS: m/z 510 (M+H)⁺; ¹H NMR (DMSO-d₆, 400 MHz): δ 1.19 (t, 3H), 2.02 (m, 2H), 2.68 (m, 4H), 3.96 (q, 2H), 6.92 (d, 2H), 7.15 (d, 1H), 7.28-7.53 (m, 6H), 7.76 (s, 1H), 8.13 (d, 1H) ppm.

### Example 144

2-Amino-5-(4-{3-[4-(2,4-dichloro-phenyl)-1-ethyl-1H-imidazol-2-yl]- propyl}-phenoxy)-benzoic acid methyl ester (105 mg, 0.2 mmol) was treated according to general procedure L using trifluoromethanesulfonic anhydride (68 μL, 0.4 mmol) and DIEA (53 μL, 0.3 mmol) till the starting material disappeared (monitored by LC-MS). The resulted mixture of di-sulfonamide and mono-sulfonamide was concentrated and treated directly as described in general procedure F to give 5-(4-{3-[4-(2,4-dichloro-phenyl)-1-ethyl-1H-imidazol-2-yl]-propyl}-phenoxy)-2-trifluoromethanesulfonylamino-benzoic acid (35 mg, 27%).

LCMS: m/z 642 (M+H)⁺; ¹H NMR (DMSO-d₆, 400 MHz): δ 1.28 (t, 3H), 2.03 (m, 2H), 2.69 (m, 4H), 3.96 (q, 2H), 6.92 (d, 2H), 7.15 (d, 1H), 7.24 (d, 2H), 7.41 (dd, 2H), 7.57 (d, 2H), 7.78 (s, 1H), 8.15 (d, 1H) ppm.

### Example 145

(5-(4-{3-[4-(2,4-dichloro-phenyl)-1-ethyl-1H-imidazol-2-yl]-propyl}-phenoxy)-2-methanesulfonylamino-benzoic acid was prepared by analagous methods to those used to prepare Example 144.

LCMS: m/z 588 (M+H)⁺.

### Example 146

4-(4-Iodo-phenyl)-butyric acid (290 mg, 1 mmol) was treated according to general procedure A using 2,4-dichlorophenacyl bromide to give 4-(2,4-dichloro-phenyl)-2-[3-(4-iodo-phenyl)-propyl]-1H-imidazole (160 mg, 34%). The resulted 1H-imidazole intermediate (140 mg, 0.3 mmol) was treated as described in general procedure E using ethyl 4-fluorobenzoate as the aryl halide and Cs₂CO₃ as the base to give 4-{4-(2,4-dichloro-phenyl)-2-[3-(4-iodo-phenyl)-propy]-imidazol-1-yl}-benzoic acid ethyl ester, which was treated as described in general procedure B using 3-(trifluoromethyl)benzeneboronic acid to give 4-{4-(2,4-dichloro-phenyl)-2-[3-(3′-trifluoromethyl-biphenyl-4-yl)-propy]-imidazol-1-yl}-benzoic acid ethyl ester, which was then treated directly according to general procedure F to give 4-{4-(2,4-dichloro-phenyl)-2-[3-(3′-trifluoromethyl-biphenyl-4-yl)-propy]-imidazol-1-yl}-benzoic acid (20 mg, 11%).

LCMS: m/z 595 (M+H)⁺; ¹H NMR (DMSO-d₆, 400 MHz): δ 2.02 (m, 2H), 2.68 (m, 4H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 147

1-(4-Methoxyphenyl)-1-cyclopropanecarboxylic acid (385 mg, 2 mmol) was treated according to general procedure A using 2,4-dichlorophenacyl bromide to give 4-(2,4-dichloro-phenyl)-2-[1-(4-methoxy-phenyl)-cyclopropyl]-1H-imidazole (230 mg, 32% yield). The resulted 1H-imidazole intermediate (216 mg, 0.6 mmol) was treated as described in general procedure E using ethyl 4-fluorobenzoate as the aryl halide and Cs₂CO₃ as the base to give 4-{4-(2,4-dichloro-phenyl)-2-[1-(4-methoxy-phenyl)-cyclopropyl]-imidazol-1-yl}-benzoic acid ethyl ester, which was treated as described in general procedure C to give 4-{4-(2,4-dichloro-phenyl)-2-[1-(4-hydroxy-phenyl)-cyclopropyl]-imidazol-1-yl}-benzoic acid ethyl ester, which was treated as described in general procedure W using 3-(trifluoromethyl)benzeneboronic acid (570 mg, 3 mmol) to give 4-(4-(2,4-dichloro-phenyl)-2-{1-[4-(3-trifluoromethyl-phenoxy)-phenyl]-cyclopropyl}-imidazol-1-yl)-benzoic acid ethyl ester, which was then treated directly according to general procedure F to give the final compound 4-(4-(2,4-dichloro-phenyl)-2-{1-[4-(3-trifluoromethyl-phenoxy)-phenyl]-cyclopropyl}-imidazol-1-yl)-benzoic acid (44 mg, 12%).

LCMS: m/z 609 (M+H)⁺; ¹H NMR (DMSO-d₆, 400 MHz): δ 1.16 (m, 2H), 1.42 (m, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 148

4-Bromoaniline (17.2 g, 0.1 mol) was heated in reflux overnight with 1H-pyrazole-1-carboxamidine hydrochloride (22.0 g, 0.15 mol) and DIEA (53 mL, 0.3 mol) in 0.5 L anhydrous THF to give N-(4-bromo-phenyl)-guanidine, which was treated according to general procedure A using 2,4-dichlorophenacyl bromide to give (4-bromo-phenyl)-[4-(2,4-dichloro-phenyl)-1H-imidazol-2-yl]-amine. The resulted 1H-imidazole intermediate was treated as described in general procedure N followed by removal of the t-butyl carbamate group of imidazole nitrogen with K₂CO₃ in MeOH at room temperature overnight to give (4-bromo-phenyl)-[4-(2,4-dichloro-phenyl)-1H-imidazol-2-yl]-carbamic acid tert-butyl ester, which was treated as described in general procedure E using methyl 4-(bromomethyl)benzoate to give 4-[2-[(4-bromo-phenyl)-tert-butoxycarbonyl-amino]-4-(2,4-dichloro-phenyl)-imidazol-1-ylmethyl]-benzoic acid methyl ester (7.6 g, 12%). LCMS: m/z 631 (M+H)⁺.

4-[2-[(4-Bromo-phenyl)-tert-butoxycarbonyl-amino]-4-(2,4-dichloro-phenyl)-imidazol-1-ylmethyl]-benzoic acid methyl ester (6.3 g, 10 mmol) was treated as described in general procedure B using 3-(trifluoromethyl)benzeneboronic acid (3.8 g, 20 mmol) to give 4-[2-[tert-butoxycarbonyl-(3′-trifluoromethyl-biphenyl-4-yl)-amino]-4-(2,4-dichloro-phenyl)-imidazol-1-ylmethyl]-benzoic acid methyl ester, which was treated according to general procedure O to give 4-[4-(2,4-dichloro-phenyl)-2-(3′-trifluoromethyl-biphenyl-4-ylamino)-imidazol-1-ylmethyl]-benzoic acid methyl ester (1.3 g, 22%).

LCMS: m/z 596 (M+H)⁺.

### Example 149

The methyl ester of Example 148 (60 mg, 0.1 mmol) was treated according to general procedure F to give 4-[4-(2,4-dichloro-phenyl)-2-(3′-trifluoromethyl-biphenyl-4-ylamino)-imidazol-1-ylmethyl]-benzoic acid (31 mg, 53%).

LCMS: m/z 582 (M+H)⁺

### Example 150

The methyl ester of Example 148 (596 mg, 1 mmol) was treated as described in general procedure P using iodomethane (63 μL, 1 mmol) to give 4-{4-(2,4-dichloro-phenyl)-2-[methyl-(3′-trifluoromethyl-biphenyl-4-yl)-amino]-imidazol-1-ylmethyl}-benzoic acid methyl ester (384 mg, 63%).

LCMS: m/z 610 (M+H)⁺.

### Example 151

The methyl ester of Example 150 (61 mg, 0.1 mmol) was treated according to general procedure F to give 4-{4-(2,4-dichloro-phenyl)-2-[methyl-(3′-trifluoromethyl-biphenyl-4-yl)-amino]-imidazol-1-ylmethyl}-benzoic acid (39 mg, 65%).

LCMS: m/z 596 (M+H)⁺; ¹H NMR (DMSO-d₆, 400 MHz): δ 2.90 (s, 3H), 5.34 (s, 2H), 7.15 (d, 2H), 7.28 (d, 2H), 7.42 (dd, 1H), 7.59 (d, 2H), 7.64 (d, 1H), 7.68-7.72 (m, 4H), 7.82 (d, 2H), 7.95 (s, 1H), 8.14 (d, 1H) ppm.

### Example 152

6-Hydroxy-1,2,3,4-tetrahydro-isoquinoline-3-carboxylic acid (2.0 g, 10 mmol) was stirred in 2N HCl/dioxane-MeOH at 100° C. for 2 hour. At completion, the reaction mixture was condensed in vacuo and the resulted 6-hydroxy-1,2,3,4-tetrahydro-isoquinoline-3-carboxylic acid methyl ester was treated directly according to general procedure N using di-tert-butyl-dicarbonate (2.6 g, 12 mmol) to give 6-hydroxy-3,4-dihydro-1H-isoquinoline-2,3-dicarboxylic acid 2-tert-butyl ester 3-methyl ester, which was treated according to general procedure W using 4-tert-butylphenylboronic acid (5.3 g, 30 mmol) to give 6-(4-tert-butyl-phenoxy)-3,4-dihydro-1H-isoquinoline-2,3-dicarboxylic acid 2-tert-butyl ester 3-methyl ester, which was treated according to general procedure O to give 6-(4-tert-butyl-phenoxy)-1,2,3,4-tetrahydro-isoquinoline-3-carboxylic acid methyl ester, which was then oxidized with 2,3-dichloro-5,6-dicyano-1,4-benzoquinone (4.5 g, 20 mmol) in anhydrous toluene (0.5 M) at 100° C. overnight (work-up procedure was similar as described in general procedure X) to give 6-(4-tert-butyl-phenoxy)-isoquinoline-3-carboxylic acid methyl ester, which was finally treated according to general procedure F to give 6-(4-tert-butyl-phenoxy)-isoquinoline-3-carboxylic acid (449 mg, 14%).

Guanidine hydrochloride (956 mg, 10 mmol) was treated according to general procedure A using 2,4-dichlorophenacyl bromide to give 4-(2,4-dichloro-phenyl)-1H-imidazol-2-ylamine (251 mg, 11%). The resulted imidazole intermediate (228 mg, 1 mmol) was treated as described in general procedure G using 6-(4-tert-butyl-phenoxy)-isoquinoline-3-carboxylic acid (321 mg, 1 mmol, prepared in the above procedure) to give 6-(4-tert-butyl-phenoxy)-isoquinoline-3-carboxylic acid [4-(2,4-dichloro-phenyl)-1H-imidazol-2-yl]-amide (181 mg, 34%). LCMS: m/z 531 (M+H)⁺.

6-(4-tert-Butyl-phenoxy)-isoquinoline-3-carboxylic acid [4-(2,4-dichloro-phenyl)-1H-imidazol-2-yl]-amide (53 mg, 0.1 mmol) was treated as described in general procedure E using methyl 4-(bromomethyl)benzoate to give 4-[2-{[6-(4-tert-butyl-phenoxy)-isoquinoline-3-carbonyl]-amino}-4-(2,4-dichloro-phenyl)-imidazol-1-ylmethyl]-benzoic acid methyl ester, which was then treated according to general procedure F to give 4-[2-{[6-(4-tert-butyl-phenoxy)-isoquinoline-3-carbonyl]-amino}-4-(2,4-dichloro-phenyl)-imidazol-1-ylmethyl]-benzoic acid (17 mg, 25%). LCMS: m/z 665 (M+H)⁺.

### Example 153

6-(4-tert-Butyl-phenoxy)-isoquinoline-3-carboxylic acid [4-(2,4-dichloro-phenyl)-1H-imidazol-2-yl]-amide (53 mg, 0.1 mmol) was treated as described in general procedure E using ethyl 4-fluorobenzoate as the aryl halide and Cs₂CO₃ as the base to give 4-[2-{[6-(4-tert-butyl-phenoxy)-isoquinoline-3-carbonyl]-amino}-4-(2,4-dichloro-phenyl)-imidazol-1-yl]-benzoic acid ethyl ester, which was then treated according to general procedure F to give 4-[2-{[6-(4-tert-butyl-phenoxy)-isoquinoline-3-carbonyl]-amino}-4-(2,4-dichloro-phenyl)-imidazol-1-yl]-benzoic acid (14 mg, 21%).

LCMS: m/z 651 (M+H)⁺.

### Example 154

3-(5-Bromo-2-methoxy-phenyl)-acrylic acid (514 mg, 2 mmol) was treated according to general procedure A using 2,4-dichlorophenacyl bromide to give 2-[2-(5-bromo-2-methoxy-phenyl)-(E)-vinyl]-4-(2,4-dichloro-phenyl)-1H-imidazole, which was treated as described in general procedure H using 1-ethynyl-4-methoxy-benzene (312 μL, 2.4 mmol) to give 4-(2,4-dichloro-phenyl)-2-{2-[2-methoxy-5-(4-methoxy-phenylethynyl)-phenyl]-(E)-vinyl}-1H-imidazole (133 mg, 14%).

LCMS: m/z 475 (M+H)⁺.

4-(2,4-Dichloro-phenyl)-2-{2-[2-methoxy-5-(4-methoxy-phenylethynyl)-phenyl]-(E)-vinyl}-1H-imidazole (95 mg, 0.2 mmol) was treated as described in general procedure E using methyl 4-(bromomethyl)benzoate to give 4-(4-(2,4-dichloro-phenyl)-2-{2-[2-methoxy-5-(4-methoxy-phenylethynyl)-phenyl]-(E)-vinyl}-imidazol-1-ylmethyl)-benzoic acid methyl ester, which was then treated according to general procedure F to give 4-(4-(2,4-dichloro-phenyl)-2-{2-[2-methoxy-5-(4-methoxy-phenylethynyl)-phenyl]-(E)-vinyl}-imidazol-1-ylmethyl)-benzoic acid (54 mg, 44%).

LCMS: m/z 609 (M+H)⁺; ¹H NMR (DMSO-d6, 400 MHz): δ 3.79 (s, 3H), 3.81 (s, 3H), 5.36 (s, 2H), 6.84 (d, 2H), 7.03 (d, 2H), 7.29 (d, 1H), 7.41-7.48 (m, 3H), 7.53 (d, 1H), 7.58 (d, 1H), 7.67-7.79 (m, 3H), 7.86 (d, 2H), 7.97 (s, 1H), 8.14 (d, 1H) ppm.

### Example 155

4-Bromocinnamic acid (predominantly trans, 22.7 g, 0.1 mol) was treated according to general procedure A using 2,4-dichlorophenacyl bromide to give 2-[2-(4-bromo-phenyl)-(E)-vinyl]-4-(2,4-dichloro-phenyl)-1H-imidazole, which was treated as described in general procedure E using bromoethane to give 2-[2-(4-bromo-phenyl)-(E)-vinyl]-4-(2,4-dichloro-phenyl)-1-ethyl-1H-imidazole. The bromo-derivative was treated as described in general procedure B using 4-methoxyphenylboronic acid (30.4 g, 0.2 mol) to give 4-(2,4-dichloro-phenyl)-1-ethyl-2-[2-(4′-methoxy-biphenyl-4-yl)-(E)-vinyl]-1H-imidazole, which was then treated as described in general procedure C to give 4′-{2-[4-(2,4-dichloro-phenyl)-1-ethyl-1H-imidazol-2-yl]-(E)-vinyl}-biphenyl-4-ol (4.8 g, 11%).

LCMS: m/z 435 (M+H)⁺.

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure W using 4-(N-boc-amino)phenylboronic acid (711 mg, 3 mmol) to give [4-(4′-{2-[4-(2,4-dichloro-phenyl)-1-ethyl-1H-imidazol-2-yl]-(E)-vinyl}-biphenyl-4-yloxy)-phenyl]-carbamic acid tert-butyl ester, which was then treated according to general procedure O to give 4-(4′-{2-[4-(2,4-dichloro-phenyl)-1-ethyl-1H-imidazol-2-yl]-(E)-vinyl}-biphenyl-4-yloxy)-phenylamine (116 mg, 22%).

LCMS: m/z 526 (M+H)⁺.

### Example 156

The amine of Example 155 (22 mg, 0.05 mmol) was treated as described in general procedure L using acetyl chloride (5 μL, 0.06 mmol) to give N-[4-(4′-{2-[4-(2,4-dichloro-phenyl)-1-ethyl-1H-imidazol-2-yl]-(E)-vinyl}-biphenyl-4-yloxy)-phenyl]-acetamide (17 mg, 60%).

LCMS: m/z 568 (M+H)⁺; ¹H NMR (DMSO-d₆, 400 MHz): δ 1.38 (t, 3H), 2.13 (s, 3H), 4.26 (q, 2H), 7.19 (d, 2H), 7.23 (d, 2H), 7.31 (d, 2H), 7.37 (d, 1H), 7.56 (d, 1H), 7.65 (d, 2H), 7.72-7.78 (m, 6H), 7.85 (s, 1H), 8.16 (d, 1H) ppm.

### Example 157

The amine of Example 155 (22 mg, 0.05 mmol) was treated as described in general procedure U using formaldehyde (37% solution in water, 15 μL, 0.2 mmol) to give [4-(4′-{2-[4-(2,4-dichloro-phenyl)-1-ethyl-1H-imidazol-2-yl]-(E)-vinyl}-biphenyl-4-yloxy)-phenyl]-dimethyl-amine (13 mg, 47%).

LCMS: m/z 554 (M+H)⁺.

### Example 158

The amine of Example 155 (44 mg, 0.1 mmol) was treated as described in general procedure L using trifluoromethanesulfonic anhydride (20 μL, 0.12 mmol) to give N-[4-(4′-{2-[4-(2,4-dichloro-phenyl)-1-ethyl-1H-imidazol-2-yl]-(E)-vinyl}-biphenyl-4-yloxy)-phenyl]-trifluoromethanesulfonamide (26 mg, 39%).

LCMS: m/z 658 (M+H)⁺.

### Example 159

The amine of Example 155 (22 mg, 0.05 mmol) was treated as described in general procedure L using trifluoromethanesulfonic anhydride (20 μL, 0.12 mmol) to give N-[4-(4′-{2[4-(2,4-dichloro-phenyl)-1-ethyl-1H-imidazol-2-yl]-(E)-vinyl}-biphenyl-4-yloxy)-phenyl]-bis(trifluoromethane)sulfonimide (12 mg, 30%).

LCMS: m/z 790 (M+H)⁺.

### Example 160

The compound of Example 158 (13 mg, 0.02 mmol) was treated as described in general procedure P using iodomethane (4 μL, 0.06 mmol) to give N-[4-(4′-{2-[4-(2,4-dichloro-phenyl)-1-ethyl-1H-imidazol-2-yl]-(E)-vinyl}-biphenyl-4-yloxy)-phenyl]-N-methyl-trifluoromethanesulfonamide (9 mg, 67%).

LCMS: m/z 672 (M+H)⁺; ¹H NMR (DMSO-d₆, 400 MHz): δ 1.38 (t, 3H), 3.46 (s, 3H), 4.28 (q, 2H), 7.19 (d, 2H), 7.23 (d, 2H), 7.31 (d, 2H), 7.37 (d, 1H), 7.56 (d, 1H), 7.65 (d, 2H), 7.70-7.77 (m, 6H), 7.84 (s, 1H), 8.15 (d, 1H) ppm.

### Example 161

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 4-fluoro-3-nitrobenzoate (299 mg, 1.5 mmol) to give 4-(4′-{2-[4-(2,4-dichloro-phenyl)-1-ethyl-1H-imidazol-2-yl]-(E)-vinyl}-biphenyl-4-yloxy)-3-nitro-benzoic acid methyl ester, which was then treated according to general procedure K to give 3-amino-4-(4′-{2-[4-(2,4-dichloro-phenyl)-1-ethyl-1H-imidazol-2-yl]-(E)-vinyl}-biphenyl-4-yloxy)-benzoic acid methyl ester (310 mg, 53%). LCMS: m/z 584 (M+H)⁺.

3-Amino-4-(4′-{2-[4-(2,4-dichloro-phenyl)-1-ethyl-1H-imidazol-2-yl]-(E)-vinyl}-biphenyl-4-yloxy)-benzoic acid methyl ester (117 mg, 0.2 mmol) was treated as described in general procedure L using benzenesulfonyl chloride (31 μL, 0.24 mmol), the resulted mixture was condensed and then treated directly as described in general procedure F to give 3-benzenesulfonylamino-4-(4′-{2-[4-(2,4-dichloro-phenyl)-1-ethyl-1H-imidazol-2-yl]-(E)-vinyl}-biphenyl-4-yloxy)-benzoic acid (51 mg, 36%).

LCMS: m/z 710 (M+H)⁺; ¹H NMR (DMSO-d₆, 400 MHz): δ 1.37 (t, 3H), 4.26 (q, 2H), 7.19 (d, 2H), 7.23 (d, 2H), 7.31 (d, 2H), 7.37 (d, 1H), 7.56 (d, 1H), 7.65 (d, 2H), 7.71-7.82 (m, 10H), 7.86 (s, 1H), 8.13 (d, 1H) ppm.

### Example 162

4-Bromocinnamic acid (predominantly trans, 22.7 g, 0.1 mol) was treated according to general procedure A using 2,4-difluorophenacyl bromide to give 2-[2-(4-bromo-phenyl)-(E)-vinyl]-4-(2,4-difluoro-phenyl)-1H-imidazole, which was treated as described in general procedure E using bromoethane to give 2-[2-(4-bromo-phenyl)-(E)-vinyl]-4-(2,4-difluoro-phenyl)-1-ethyl-1H-imidazole, which was treated as described in general procedure B using 4-methoxyphenylboronic acid (30.4 g, 0.2 mol) to give 4-(2,4-difluoro-phenyl)-1-ethyl-2-[2-(4′-methoxy-biphenyl-4-yl)-(E)-vinyl]-1H-imidazole, which was then treated as described in general procedure C to give 4′-{2-[4-(2,4-difluoro-phenyl)-1-ethyl-1H-imidazol-2-yl]-(E)-vinyl}-biphenyl-4-ol (4.8 g, 12%). LCMS: m/z 403 (M+H)⁺.

4′-{2-[4-(2,4-Difluoro-phenyl)-1-ethyl-1H-imidazol-2-yl]-(E)-vinyl}-biphenyl-4-ol (804 mg, 2 mmol) was treated as described in general procedure J using methyl 2-amino-5-bromobenzoate (691 mg, 3 mmol) to give 4-(4′-{2-[4-(2,4-dichloro-phenyl)-1-ethyl-1H-imidazol-2-yl]-(E)-vinyl}-biphenyl-4-yloxy)-3-nitro-benzoic acid methyl ester, which was then treated according to general procedure K to give 2-amino-5-(4′-{2-[4-(2,4-difluoro-phenyl)-1-ethyl-1H-imidazol-2-yl]-(E)-vinyl}-biphenyl-4-yloxy)-benzoic acid methyl ester (265 mg, 24%). LCMS: m/z 552 (M+H)⁺.

2-Amino-5-(4′-{2-[4-(2,4-difluoro-phenyl)-1-ethyl-1H-imidazol-2-yl]-(E)-vinyl}-biphenyl-4-yloxy)-benzoic acid methyl ester (110 mg, 0.2 mmol) was treated as described in general procedure L using methanesulfonyl chloride (16 μL, 0.2 mmol) to give 5-(4′-{2-[4-(2,4-difluoro-phenyl)-1-ethyl-1H-imidazol-2-yl]-(E)-vinyl}-biphenyl-4-yloxy)-2-methanesulfonylamino-benzoic acid methyl ester (49 mg, 39%).

LCMS: m/z 630 (M+H)⁺; ¹H NMR (DMSO-d₆, 400 MHz): δ 1.38 (t, 3H), 3.15 (s, 3H), 3.79 (s, 3H), 4.28 (q, 2H), 7.19 (d, 2H), 7.23 (d, 2H), 7.31 (d, 2H), 7.37 (d, 1H), 7.56 (d, 1H), 7.65 (d, 2H), 7.72-7.84 (m, 6H), 8.14 (d, 1H) ppm.

### Example 163

5-(4′-{2-[4-(2,4-Difluoro-phenyl)-1-ethyl-1H-imidazol-2-yl]-(E)-vinyl}-biphenyl-4-yloxy)-2-methanesulfonylamino-benzoic acid methyl ester (38 mg, 0.06 mmol) was treated as described in general procedure F to give 5-(4′-{2-[4-(2,4-difluoro-phenyl)-1-ethyl-1H-imidazol-2-yl]-(E)-vinyl}-biphenyl-4-yloxy)-2-methanesulfonylamino-benzoic acid (22 mg, 59%).

LCMS: m/z 616 (M+H)⁺; ¹H NMR (DMSO-d₆, 400 MHz): δ 1.37 (t, 3H), 3.15 (s, 3H), 4.28 (q, 2H), 7.19 (d, 2H), 7.23 (d, 2H), 7.31 (d, 2H), 7.37 (d, 1H), 7.56 (d, 1H), 7.63 (d, 2H), 7.71-7.83 (m, 6H), 8.15 (d, 1H) ppm.

### Example 164

4-Bromocinnamic acid (predominantly trans, 11.4 g, 0.05 mol) was treated according to general procedure A using 2,4-difluorophenacyl bromide to give 2-[2-(4-bromo-phenyl)-(E)-vinyl]-4-(2,4-difluoro-phenyl)-1H-imidazole, which was treated as described in general procedure B with 3-(trifluoromethyl)benzeneboronic acid (19 g, 0.1 mol) to give 4-(2,4-difluoro-phenyl)-2-[2-(3′-trifluoromethyl-biphenyl-4-yl)-(E)-vinyl]-1H-imidazole (4.5 g, 21%). LCMS: m/z 427 (M+H)⁺

4-(2,4-Difluoro-phenyl)-2-[2-(3′-trifluoromethyl-biphenyl-4-yl)-(E)-vinyl]-1H-imidazole (852 mg, 2 mmol) was treated as described in general procedure E using 4-nitrobenzyl bromide (864 mg, 4 mmol) to give 4-(2,4-difluoro-phenyl)-1-(4-nitro-benzyl)-2-[2-(3′-trifluoromethyl-biphenyl-4-yl)-(E)-vinyl]-1H-imidazole, which was treated as described in general procedures Y1 and Y2 [using methyl bromoacetate (227 μL, 2.4 mmol)] to give (4-{4-(2,4-difluoro-phenyl)-2-[2-(3′-trifluoromethyl-biphenyl-4-yl)-(E)-vinyl]-imidazol-1-ylmethyl}-phenylamino)-acetic acid methyl ester, which was then treated as described in general procedure F to give (4-{4-(2,4-difluoro-phenyl)-2-[2-(3′-trifluoromethyl-biphenyl-4-yl)-(E)-vinyl]-imidazol-1-ylmethyl}-phenylamino)-acetic acid (165 mg, 14%).

LCMS: m/z 590 (M+H)⁺; ¹H NMR (DMSO-d₆, 400 MHz): δ 3.89 (d, 2H), 5.62 (s, 2H), 7.19 (d, 2H), 7.23 (d, 2H), 7.31 (d, 2H), 7.37 (d, 1H), 7.56 (d, 1H), 7.68-7.83 (m, 9H), 8.14 (d, 1H) ppm.

### Example 165

4-(2,4-Difluoro-phenyl)-2-[2-(3′-trifluoromethyl-biphenyl-4-yl)-(E)-vinyl]-1H-imidazole (852 mg, 2 mmol) was treated as described in general procedure E using 4-nitrobenzyl bromide (864 mg, 4 mmol) to give 4-(2,4-difluoro-phenyl)-1-(4-nitro-benzyl) 2-[2-(3′-trifluoromethyl-biphenyl-4-yl)-(E)-vinyl]-1H-imidazole, which was treated as described in general procedure Y using methyl bromoacetate (227 μL, 2.4 mmol) in procedure Y2 to give 5-(4-{4-(2,4-difluoro-phenyl)-2-[2-(3′-trifluoromethyl-biphenyl-4-yl)-(E)-vinyl]-imidazol-1-ylmethyl}-phenyl)-1,2,5-thiadiazolidine-3-one-1,1-dioxide (65 mg, 5%).

LCMS: m/z 651 (M+H)⁺; ¹H NMR (DMSO-d₆, 400 MHz): δ 3.86 (s, 2H), 5.39 (s, 2H), 7.19 (d, 2H), 7.23 (d, 2H), 7.31 (d, 2H), 7.37 (d, 1H), 7.56 (d, 1H), 7.68-7.83 (m, 9H), 8.13 (d, 1H) ppm.

### Example 166

4-{4-(2,4-Dichloro-phenyl)-2-[2-(3′-trifluoromethyl-biphenyl-4-yl)-(E)-vinyl]-imidazol-1-ylmethyl}-benzoic acid (59 mg, 0.1 mmol) was treated as described in general procedure G using glycine methyl ester hydrochloride (13 mg, 0.1 mmol) to give (4-{4-(2,4-dichloro-phenyl)-2-[2-(3′-trifluoromethyl-biphenyl-4-yl)-(E)-vinyl]-imidazol-1-ylmethyl}-benzoylamino)-acetic acid methyl ester, which was then treated as described in general procedure F to give (4-{4-(2,4-dichloro-phenyl)-2-[2-(3′-trifluoromethyl-biphenyl-4-yl)-(E)-vinyl]-imidazol-1-ylmethyl}-benzoylamino)-acetic acid (35 mg, 54%).

LCMS: m/z 650 (M+H)⁺; ¹H NMR (DMSO-d₆, 400 MHz): δ 3.88 (d, 2H), 5.62 (s, 2H), 7.19 (d, 2H), 7.23 (d, 2H), 7.31 (d, 2H), 7.37 (d, 1H), 7.56 (d, 1H), 7.66-7.82 (m, 9H), 8.15 (d, 1H) ppm.

### Example 167

4-{4-(2,4-Dichloro-phenyl)-2-[2-(3′-trifluoromethyl-biphenyl-4-yl)-(E)-vinyl]-imidazol-1-ylmethyl}-benzoic acid (59 mg, 0.1 mmol) was treated as described in general procedure G using sarcosine methyl ester hydrochloride (15 mg, 0.1 mmol) to give [(4-{4-(2,4-dichloro-phenyl)-2-[2-(3′-trifluoromethyl-biphenyl-4-yl)-(E)-vinyl]-imidazol-1-ylmethyl}-benzoyl)-methyl-amino]-acetic acid methyl ester, which was then treated as described in general procedure F to give [(4-{4-(2,4-dichloro-phenyl)-2-[2-(3′-trifluoromethyl-biphenyl-4-yl)-(E)-vinyl]-imidazol-1-ylmethyl}-benzoyl)-methyl-amino]-acetic acid (38 mg, 57%).

LCMS: m/z 664 (M+H)⁺; ¹H NMR (DMSO-d₆, 400 MHz): δ 2.96 (s, 3H), 3.88 (s, 2H), 5.62 (s, 2H), 7.19 (d, 2H), 7.23 (d, 2H), 7.31 (d, 2H), 7.37 (d, 1H), 7.56 (d, 1H), 7.66-7.82 (m, 9H), 8.14 (d, 1H) ppm.

### Example 168

4-{4-(2,4-Dichloro-phenyl)-2-[2-(3′-trifluoromethyl-biphenyl-4-yl)-(E)-vinyl]-imidazol-1-ylmethyl}-benzoic acid (593 mg, 1 mmol) was stirred with oxalyl chloride (873 μL, 10 mmol) in 5 mL dry DCM with 1 drop of DMF at 80° C. under nitrogen for 2 hours. After cooling, the reaction mixture was condensed and dissolved in 5 mL anhydrous THF and cooled down to −20° C. To this solution was added 1.5 mL lithium diisopropylamide (2M) and stirred at −20° C. under nitrogen for 1 hour, then anhydrous ethyl acetate (118 μL, 1.2 mmol) was added, and the reaction mixture was left to warm up to room temperature and stirred overnight. At completion the reaction mixture was quenched with water/EtOAc and the layers separated. The aqueous layer was further extracted with EtOAc, and the organic layers combined and dried over Na₂SO₄. The solvent was removed in vacuo and the residue was purified by silica gel chromatography to yield 3-(4-{4-(2,4-dichloro-phenyl)-2-[2-(3′-trifluoromethyl-biphenyl-4-yl)-(E)-vinyl]-imidazol-1-ylmethyl}-phenyl)-3-oxo-propionic acid ethyl ester (179 mg, 27%).

LCMS: m/z 663 (M+H)⁺.

3-(4-{4-(2,4-Dichloro-phenyl)-2-[2-(3′-trifluoromethyl-biphenyl-4-yl)-(E)-vinyl]-imidazol-1-ylmethyl}-phenyl)-3-oxo-propionic acid ethyl ester (66 mg, 0.1 mmol) was stirred with hydrazine dihydrochloride (105 mg, 1 mmol) in 1 mL dry EtOH with at 80° C. under nitrogen overnight. At completion the reaction mixture was diluted with water/EtOAc and the layers separated. The aqueous layer was further extracted with EtOAc, and the organic layers combined and dried over Na₂SO₄. The solvent was removed in vacuo and the residue was purified by silica gel chromatography to yield 5-(4-{4-(2,4-dichloro-phenyl)-2-[2-(3′-trifluoromethyl-biphenyl-4-yl)-(E)-vinyl]-imidazol-1-ylmethyl}-phenyl)-1H-pyrazol-3-ol (8 mg, 12% yield).

LCMS: m/z 631 (M+H)⁺; ¹H NMR (DMSO-d₆, 400 MHz): δ 5.43 (s, 2H), 7.19 (d, 2H), 7.23 (d, 2H), 7.31 (d, 2H), 7.37 (d, 1H), 7.56 (d, 1H), 7.65-7.84 (m, 10H), 8.14 (d, 1H) ppm.

### Example 169

3-(4-{4-(2,4-Dichloro-phenyl)-2-[2-(3′-trifluoromethyl-biphenyl-4-yl)-(E)-vinyl]-imidazol-1-ylmethyl}-phenyl)-3-oxo-propionic acid ethyl ester (66 mg, 0.1 mmol) was stirred with hydrazine dihydrochloride (105 mg, 1 mmol) in 1 mL dry EtOH with at 80° C. under nitrogen overnight. At completion, the reaction mixture was diluted with water/EtOAc and the layers were separated. The aqueous layer was further extracted with EtOAc, and the organic layers combined and dried over Na₂SO₄. The solvent was removed in vacuo and the residue was purified by silica gel chromatography to yield 5-(4-{4-(2,4-dichloro-phenyl)-2-[2-(3′-trifluoromethyl-biphenyl-4-yl)-(E)-vinyl]-imidazol-1-ylmethyl}-phenyl)-3-ethoxy-1H-pyrazole as the less-polar by-product (12 mg, 18%).

LCMS: m/z 659 (M+H)⁺.

### Example 170

3-(4-{4-(2,4-Dichloro-phenyl)-2-[2-(3′-trifluoromethyl-biphenyl-4-yl)-(E)-vinyl]-imidazol-1-ylmethyl}-phenyl)-3-oxo-propionic acid ethyl ester (66 mg, 0.1 mmol) was stirred with hydroxylamine hydrochloride (70 mg, 1 mmol) in 1 mL dry EtOH with at 80° C. under nitrogen overnight. At completion the reaction mixture was diluted with water/EtOAc and the layers were separated. The aqueous layer was further extracted with EtOAc, and the organic layers combined and dried over Na₂SO₄. The solvent was removed in vacuo and the residue was purified by silica gel chromatography to yield 5-(4-{4-(2,4-dichloro-phenyl)-2-[2-(3′-trifluoromethyl-biphenyl-4-yl)-(E)-vinyl]-imidazol-1-ylmethyl}-phenyl) -isoxazol-3-ol (14 mg, 22%).

LCMS: m/z 632 (M+H)⁺; ¹H NMR (DMSO-d₆, 400 MHz): δ 5.44 (s, 2H), 7.19 (d, 2H), 7.23 (d, 2H), 7.31 (d, 2H), 7.37 (d, 1H), 7.56 (d, 1H), 7.64-7.85 (m, 10H), 8.15 (d, 1H) ppm.

### Example 171

4-(2,4-Dichloro-phenyl)-2-[2-(3′-trifluoromethyl-biphenyl-4-yl)-(E)-vinyl]-1H-imidazole (919 mg, 2 mmol) was treated as described in general procedure E using 4-nitrobenzyl bromide (864 mg, 4 mmol) to give 4-(2,4-dichloro-phenyl)-1-(4-nitro-benzyl)-2-[2-(3′-trifluoromethyl-biphenyl-4-yl)-(E)-vinyl]-1H-imidazole, which in turn was reduced as described in general procedure K to give 4-{4-(2,4-dichloro-phenyl)-2-[2-(3′-trifluoromethyl-biphenyl-4-yl)-(E)-vinyl]-imidazol-1-ylmethyl}-phenylamine (802 mg, 71%).

LCMS: m/z 564 (M+H)⁺.

4-{4-(2,4-Dichloro-phenyl)-2-[2-(3′-trifluoromethyl-biphenyl-4-yl)-(E)-vinyl]-imidazol-1-ylmethyl}-phenylamine (564 mg, 1 mmol) was treated as described in general procedures Y2 [using methyl bromoacetate (114 μL, 1.2 mmol)] and Y3 [using N-(chlorocarbonyl) isocyanate (121 μL, 1.5 mmol) instead of chlorosulfonyl isocyanate] to give 1-(4-{4-(2,4-dichloro-phenyl)-2-[2-(3′-trifluoromethyl-biphenyl-4-yl)-(E)-vinyl]-imidazol-1-ylmethyl}-phenyl)-imidazolidine-2,4-dione (39 mg, 6%).

LCMS: m/z 647 (M+H)⁺; ¹H NMR (DMSO-d₆, 400 MHz): δ 3.85 (s, 2H), 5.36 (s, 2H), 7.19 (d, 2H), 7.23 (d, 2H), 7.31 (d, 2H), 7.37 (d, 1H), 7.56 (d, 1H), 7.68-7.83 (m, 9H), 8.14 (d, 1H) ppm.

### Example 172

4-{4-(2,4-Dichloro-phenyl)-2-[2-(3′-trifluoromethyl-biphenyl-4-yl)-(E)-vinyl]-imidazol-1-ylmethyl}-phenylamine (57 mg, 0.1 mmol) was stirred with 1,1′-carbonyldiimidazole (20 mg, 0.12 mmol) and 4-(dimethylamino)pyridine (3 mg, 0.02 mmol) in 1 mL dry DCE at 80° C. under nitrogen for 1 hour, then glycine methyl ester (11 mg, 0.12 mmol) was added, and the reaction mixture was stirred at 80° C. under nitrogen for 1 hour. The solvent was removed in vacuo and the residue was purified by silica gel chromatography to yield [3-(4-{4-(2,4-dichloro-phenyl)-2-[2-(3′-trifluoromethyl-biphenyl-4-yl)-(E)-vinyl]-imidazol-1-ylmethyl}-phenyl)-ureido]-acetic acid methyl ester (28 mg, 41%).

LCMS: m/z 679 (M+H)⁺.

### Example 173

The methyl ester of Example 172 (21 mg, 0.03 mmol) was treated as described in general procedure F to give [3-(4-{4-(2,4-dichloro-phenyl)-2-[2-(3′-trifluoromethyl-biphenyl-4-yl)-(E)-vinyl]-imidazol-1-ylmethyl}-phenyl)-ureido]-acetic acid (15 mg, 75%).

LCMS: m/z 665 (M+H)⁺; ¹H NMR (DMSO-d₆, 400 MHz): δ 3.88 (d, 2H), 5.46 (s, 2H), 7.19 (d, 2H), 7.23 (d, 2H), 7.31 (d, 2H), 7.37 (d, 1H), 7.56 (d, 1H), 7.68-7.83 (m, 9H), 8.14 (d, 1H) ppm.

### Example 174

4-{4-(2,4-Dichloro-phenyl)-2-[2-(3′-trifluoromethyl-biphenyl-4-yl)-(E)-vinyl]-imidazol-1-ylmethyl}-phenylamine (57 mg, 0.1 mmol) was stirred with 1,1′-carbonyldiimidazole (20 mg, 0.12 mmol) and 4-(dimethylamino)pyridine (3 mg, 0.02 mmol) in 1 mL dry DCE at 80° C. under nitrogen for 1 hour, then sarcosine methyl ester (13 mg, 0.12 mmol) was added, and the reaction mixture was stirred at 80° C. under nitrogen for 1 hour. The solvent was removed in vacuo and the residue was purified by silica gel chromatography to yield [3-(4-{4-(2,4-dichloro-phenyl)-2-[2-(3′-trifluoromethyl-biphenyl-4-yl)-(E)-vinyl]-imidazol-1-ylmethyl}-phenyl)-1-methyl-ureido]-acetic acid methyl ester (32 mg, 46%).

LCMS: m/z 693 (M+H)⁺.

### Example 175

The methyl ester of Example 174 (21 mg, 0.03 mmol) was treated as described in general procedure F to give [3-(4-{4-(2,4-dichloro-phenyl)-2-[2-(3′-trifluoromethyl-biphenyl-4-yl)-(E)-vinyl]-imidazol-1-ylmethyl}-phenyl)-1-methyl-ureido]-acetic acid (14 mg, 69%).

LCMS: m/z 679 (M+H)⁺; ¹H NMR (DMSO-d₆, 400 MHz): δ 2.97 (s, 3H), 3.89 (s, 2H), 5.48 (s, 2H), 7.19 (d, 2H), 7.23 (d, 2H), 7.31 (d, 2H), 7.37 (d, 1H), 7.56 (d, 1H), 7.68-7.83 (m, 9H), 8.15 (d, 1H) ppm.

### Example 176

4-{4-(2,4-Dichloro-phenyl)-2-[2-(3′-trifluoromethyl-biphenyl-4-yl)-(E)-vinyl]-imidazol-1-ylmethyl}-phenylamine (564 mg, 1 mmol) was treated as described in general procedures Y2 [using methyl-α-bromoisobutyrate (647 μL, 5 mmol)] and Y3 to give 5-(4-{4-(2,4-dichloro-phenyl)-2-[2-(3′-trifluoromethyl-biphenyl-4-yl)-(E)-vinyl]-imidazol-1-ylmethyl}-phenyl)-4,4-dimethyl-1,2,5-thiadiazolidine-3-one-1,1-dioxide (43 mg, 6%).

LCMS: m/z 711 (M+H)⁺; ¹H NMR (DMSO-d₆, 400 MHz): δ 1.93 (s, 6H), 5.37 (s, 2H), 7.17 (d, 2H), 7.23 (d, 2H), 7.31 (d, 2H), 7.37 (d, 1H), 7.56 (d, 1H), 7.66-7.81 (m, 9H), 8.13 (d, 1H) ppm.

### Example 177

The compound of Example 176 (29 mg, 0.04 mmol) was treated as described in general procedure Z using iodomethane (4 μL, 0.06 mmol) to give 5-(4-{4-(2,4-dichloro-phenyl)-2-[2-(3′-trifluoromethyl-biphenyl-4-yl)-(E)-vinyl]-imidazol-1-ylmethyl}-phenyl)-2,4,4-trimethyl-1,2,5-thiadiazolidine-3-one-1,1-dioxide (10 mg, 35% yield).

LCMS: m/z 725 (M+H)⁺; ¹H NMR (DMSO-d₆, 400 MHz): δ 1.94 (s, 6H), 2.94 (s, 3H), 5.34 (s, 2H), 7.18 (d, 2H), 7.23 (d, 2H), 7.31 (d, 2H), 7.37 (d, 1H), 7.56 (d, 1H), 7.66-7.82 (m, 9H), 8.14 (d, 1H) ppm.

### Example 178

4-Bromocinnamic acid (predominantly trans, 227 mg, 1 mmol) was treated according to general procedure A using phenacyl bromide to give 2-[2-(4-bromo-phenyl)-(E)-vinyl]-4-phenyl-1H-imidazole, which was treated as described in general procedure B using 3-(trifluoromethyl)benzeneboronic acid to give 4-phenyl-2-[2-(3′-trifluoromethyl-biphenyl-4-yl)-(E)-vinyl]-1H-imidazole, which was treated as described in general procedure E using 4-nitrobenzyl bromide to give 1-(4-nitro-benzyl)-4-phenyl-2-[2-(3′-trifluoromethyl-biphenyl-4-yl)-(E)-vinyl]-1H-imidazole. The nitro-substituted compound was treated as described in general procedures Y1, Y2 and Y3 (using methyl bromoacetate in Y2) to give 5-(4-{4-phenyl-2-[2-(3′-trifluoromethyl-biphenyl-4-yl)-(E)-vinyl]-imidazol-1-ylmethyl}-phenyl)-1,2,5-thiadiazolidine-3-one-1,1-dioxide (18 mg, 3%).

LCMS: m/z 615 (M+H)⁺; ¹H NMR (DMSO-d₆, 400 MHz): δ 3.86 (s, 2H), 5.40 (s, 2H), 7.19 (d, 2H), 7.23 (d, 2H), 7.31 (d, 2H), 7.37 (d, 1H), 7.56 (d, 1H), 7.68-8.01 (m, 12H) ppm.

### Example 179

4-Bromocinnamic acid (predominantly trans, 227 mg, 1 mmol) was treated according to general procedure A using 2-chlorophenacyl bromide to give 2-[2-(4-bromo-phenyl)-(E)-vinyl]-4-(2-chloro-phenyl)-1H-imidazole, which was treated as described in general procedure B using 3-(trifluoromethyl)benzeneboronic acid to give 4-(2-chloro-phenyl)-2-[2-(3′-trifluoromethyl-biphenyl-4-yl)-(E)-vinyl]-1H-imidazole. The imidazole derivative was treated as described in general procedure E using 4-nitrobenzyl bromide to give 4-(2-chloro-phenyl)-1-(4-nitro-benzyl)-2-[2-(3′-trifluoromethyl-biphenyl-4-yl)-(E)-vinyl]-1H-imidazole, which was treated as described in general procedures Y1, Y2 and Y3 (using methyl bromoacetate in Y2) to give 5-(4-{4-(2-chloro-phenyl)-2-[2-(3′-trifluoromethyl-biphenyl-4-yl)-(E)-vinyl]-imidazol-1-ylmethyl}-phenyl)-1,2,5-thiadiazolidine-3-one-1,1-dioxide (26 mg, 4%).

LCMS: m/z 649 (M+H)⁺; ¹H NMR (DMSO-d₆, 400 MHz): δ 3.87 (s, 2H), 5.42 (s, 2H), 7.19 (d, 2H), 7.23 (d, 2H), 7.31 (d, 2H), 7.37 (d, 1H), 7.56 (d, 1H), 7.70-8.07 (m, 11H) ppm.

### Example 180

5-(4-{4-(4-chloro-phenyl)-2-[2-(3′-trifluoromethyl-biphenyl-4-yl)-(E)-vinyl]-imidazol-1-ylmethyl}-phenyl)-1,2,5-thiadiazolidine-3-one-1,1-dioxide was prepared was prepared by analagous methods to those used to prepare Example 179. LCMS: m/z 649 (M+H)⁺.

### Example 181

4-Bromocinnamic acid (predominantly trans, 227 mg, 1 mmol) was treated according to general procedure A using 4-chlorophenacyl bromide to give 2-[2-(4-bromo-phenyl)-(E)-vinyl]-4-(4-chloro-phenyl)-1H-imidazole, which was treated as described in general procedure B using 3-(trifluoromethyl)benzeneboronic acid to give 4-(4-chloro-phenyl)-2-[2-(3′-trifluoromethyl-biphenyl-4-yl)-(E)-vinyl]-1H-imidazole. The imidazole derivative was treated as described in general procedure E using methyl 4-(bromomethyl)benzoate to give 4-{4-(4-chloro-phenyl)-2-[2-(3′-trifluoromethyl-biphenyl-4-yl)-(E)-vinyl]-imidazol-1-ylmethyl}-benzoic acid methyl ester, which was then treated as described in general procedure F to give 4-{4-(4-chloro-phenyl)-2-[2-(3′-trifluoromethyl-biphenyl-4-yl)-(E)-vinyl]-imidazol-1-ylmethyl}-benzoic acid (78 mg, 14%).

LCMS: m/z 559 (M+H)⁺; ¹H NMR (DMSO-d₆, 400 MHz): δ 5.63 (s, 2H), 7.18 (d, 2H), 7.24 (d, 2H), 7.32 (d, 2H), 7.36 (d, 1H), 7.57 (d, 1H), 7.72-8.08 (m, 11H) ppm.

By analagous methods to those used to prepare Example 181, the following compounds were synthesized:

| Example | Name | LC/MS (m/z) |
|---|---|---|
| 182 | 4-{4-(2-Chloro-phenyl)-2-[2-(3′- | 559 (M + H)⁺ |
| | trifluoromethyl-biphenyl-4-yl)-(E)- | |
| | vinyl]-imidazol-1-ylmethyl}-benzoic | |
| | acid | |
| 183 | 4-{4-(2,6-Dichloro-phenyl)-2-[2-(3′- | 593 (M + H)⁺ |
| | trifluoromethyl-biphenyl-4-yl)-(E)-vinyl]- | |
| | imidazol-1-ylmethyl}-benzoic acid | |
| 184 | 4-{4-(3,4-Dichloro-phenyl)-2-[2-(3′- | 593 (M + H)⁺ |
| | trifluoromethyl-biphenyl-4-yl)-(E)-vinyl]- | |
| | imidazol-1-ylmethyl}-benzoic acid | |
| 185 | 4-{4-(3,4-Difluoro-phenyl)-2-[2-(3′- | 561 (M + H)⁺ |
| | trifluoromethyl-biphenyl-4-yl)-(E)-vinyl]- | |
| | imidazol-1-ylmethyl}-benzoic acid | |
| 186 | 4-{4-(2-chloro-4-fluoro-phenyl)-2-[2-(3′- | 577 (M + H)⁺ |
| | trifluoromethyl-biphenyl-4-yl)-(E)-vinyl]- | |
| | imidazol-1-ylmethyl}-benzoic acid | |

### Example 187

4-[2-[2-(4-Bromo-phenyl)-(E)-vinyl]-4-(2,4-dichloro-phenyl)-imidazol-1-ylmethyl]-benzoic acid methyl ester (542 mg, 1 mmol) was treated as described in general procedure B using 4-isopropyloxyphenylboronic acid (360 mg, 2 mmol) to give 4-{4-(2,4-dichloro-phenyl)-2-[2-(4′-isopropoxy-biphenyl-4-yl)-(E)-vinyl]-imidazol-1-ylmethyl}-benzoic acid methyl ester, which was then treated as described in general procedure F to give 4-{4-(2,4-dichloro-phenyl)-2-[2-(4′-isopropoxy-biphenyl-4-yl)-(E)-vinyl]-imidazol-1-ylmethyl}-benzoic acid (204 mg, 35%).

LCMS: m/z 583 (M+H)⁺; ¹H NMR (DMSO-d₆, 400 MHz): δ 1.27 (d, 6H), 4.66 (m, 1H), 5.64 (s, 2H), 6.89 (d, 2H), 7.12 (d, 2H), 7.32 (d, 2H), 7.37 (d, 1H), 7.57 (d, 1H), 7.64-7.97 (m, 9H), 8.13 (d, 1H) ppm.

### Example 188

4-[2-[2-(4-Bromo-phenyl)-(E)-vinyl]-4-(2,4-dichloro-phenyl)-imidazol-1-ylmethyl]-benzoic acid methyl ester (542 mg, 1 mmol) was treated as described in general procedure B using 2-fluoro-5-(trifluoromethyl)phenylboronic acid (416 mg, 2 mmol) to give 4-{4-(2,4-dichloro-phenyl)-2-[2-(2′-fluoro-5′-trifluoromethyl-biphenyl-4-yl)-(E)-vinyl]-imidazol-1-ylmethyl}-benzoic acid methyl ester, which was then treated as described in general procedure F to give 4-{4-(2,4-dichloro-phenyl)-2-[2-(2′-fluoro-5′-trifluoromethyl-biphenyl-4-yl)-(E)-vinyl]-imidazol-1-ylmethyl}-benzoic acid (287 mg, 47%).

LCMS: m/z 611 (M+H)⁺; ¹H NMR (DMSO-d₆, 400 MHz): δ 5.75 (s, 2H), 7.13 (d, 2H), 7.24 (d, 2H), 7.32 (d, 2H), 7.37 (d, 1H), 7.57 (d, 1H), 7.64-8.04 (m, 8H), 8.14 (d, 1H) ppm.

### Example 189

4-(2,6-dichloro-phenyl)-2-[2-(3′-trifluoromethyl-biphenyl-4-yl)-(E)-vinyl]-1H-imidazole was treated as described in general procedure E using 4-nitrobenzyl bromide to give 4-(2,6-dichloro-phenyl)-1-(4-nitro-benzyl)-2-[2-(3′-trifluoromethyl-biphenyl-4-yl)-(E)-vinyl]-1H-imidazole, which was then treated as described in general procedures Y1 to Y2 (using methyl bromoacetate) to give (4-{4-(2,6-dichloro-phenyl)-2-[2-(3′-trifluoromethyl-biphenyl-4-yl)-(E)-vinyl]-imidazol-1-ylmethyl}-phenylamino)-acetic acid methyl ester. The ester was hydrolyzed as described in general procedure F to give (4-{4-(2,6-dichloro-phenyl)-2-[2-(3′-trifluoromethyl-biphenyl-4-yl)-(E)-vinyl]-imidazol-1-ylmethyl}-phenylamino)-acetic acid (68 mg, 11%).

LCMS: m/z 622 (M+H)⁺; ¹H NMR (DMSO-d₆, 400 MHz): δ 3.88 (d, 2H), 5.62 (s, 2H), 7.17 (d, 2H), 7.24 (d, 2H), 7.32 (d, 2H), 7.36 (d, 1H), 7.57 (d, 1H), 7.71-8.10 (m, 10H) ppm.

### Example 190

4-(2,6-dichloro-phenyl)-2-[2-(3′-trifluoromethyl-biphenyl-4-yl)-(E)-vinyl]-1H-imidazole (XX mg, XX mmol) was treated as described in general procedure E using 4-nitrobenzyl bromide to give 4-(2,6-dichloro-phenyl)-1-(4-nitro-benzyl)-2-[2-(3′-trifluoromethyl-biphenyl-4-yl)-(E)-vinyl]-1H-imidazole, which was then treated as described in general procedure Y (using methyl bromoacetate in Y2) to give 5-(4-{4-(2,6-dichloro-phenyl)-2-[2-(3′-trifluoromethyl-biphenyl-4-yl)-(E)-vinyl]-imidazol-1-ylmethyl}-phenyl)-1,2,5-thiadiazolidine-3-one-1,1-dioxide (20 mg, 3%).

LCMS: m/z 683 (M+H)⁺; ¹H NMR (DMSO-d₆, 400 MHz): δ 3.87 (s, 2H), 5.43 (s, 2H), 7.16 (d, 2H), 7.24 (d, 2H), 7.32 (d, 2H), 7.36 (d, 1H), 7.57 (d, 1H), 7.70-8.09 (m, 10H) ppm.

By analagous methods to those used to prepare Example 190, the following compounds were synthesized:

| Example | Name | LC/MS (m/z) |
|---|---|---|
| 191 | 5-(4-{4-(3,4-Dichloro-phenyl)-2-[2-(3′- | 683 (M + H)⁺ |
| | trifluoromethyl-biphenyl-4-yl)-(E)-vinyl]- | |
| | imidazol-1-ylmethyl}-phenyl)-1,2,5- | |
| | thiadiazolidine-3-one-1,1-dioxide | |
| 192 | 5-(4-{4-(3,4-Difluoro-phenyl)-2-[2-(3′- | 651 (M + H)⁺ |
| | trifluoromethyl-biphenyl-4-yl)-(E)-vinyl]- | |
| | imidazol-1-ylmethyl}-phenyl)-1,2,5- | |
| | thiadiazolidine-3-one-1,1-dioxide | |
| 193 | 5-(4-{4-(2-Chloro-4-fluoro-phenyl)-2-[2-(3′- | 667 (M + H)⁺ |
| | trifluoromethyl-biphenyl-4-yl)-(E)-vinyl]- | |
| | imidazol-1-ylmethyl}-phenyl)-1,2,5- | |
| | thiadiazolidine-3-one-1,1-dioxide | |

### Example 194

4-Bromocinnamic acid (predominantly trans, 227 mg, 1 mmol) was treated according to general procedure A using 2,4-difluorophenacyl bromide to give 2-[2-(4-bromo-phenyl)-(E)-vinyl]-4-(2,4-difluoro-phenyl)-1H-imidazole, which was treated as described in general procedure E using ethyl 4-fluorobenzoate as the aryl halide and Cs₂CO₃ as the base to give 4-[2-[2-(4-bromo-phenyl)-(E)-vinyl]-4-(2,4-difluoro-phenyl)-imidazol-1-yl]-benzoic acid ethyl ester. The bromo-ester was treated as described in general procedure B using 3-(methylsulfonylphenyl)boronic acid to give 4-{4-(2,4-difluoro-phenyl)-2-[2-(3′-methanesulfonyl-biphenyl-4-yl)-(E)-vinyl]-imidazol-1-yl}-benzoic acid ethyl ester, which was then treated as described in general procedure F to give 4-{4-(2,4-difluoro-phenyl)-2-[2-(3′-methanesulfonyl-biphenyl-4-yl)-(E)-vinyl]-imidazol-1-yl}-benzoic acid (22 mg, 4%).

LCMS: m/z 557 (M+H)⁺; ¹H NMR (DMSO-d₆, 400 MHz): δ 3.28 (s, 3H), 7.16 (d, 2H), 7.24 (d, 2H), 7.32 (d, 2H), 7.36 (d, 1H), 7.57 (d, 1H), 7.71-8.13 (m, 10H) ppm.

### Example 195

4-{4-(3,4-dichloro-phenyl)-2-[2-(3′-methanesulfonyl-biphenyl-4-yl)-(E)-vinyl]-imidazol-1-yl}-benzoic acid was prepared was prepared by analagous methods to those used to prepare Example 194. LCMS: m/z 589 (M+H)⁺.

### Example 196

4′-{2-[4-(2,4-Dichloro-phenyl)-1-ethyl-1H-imidazol-2-yl]-(E)-vinyl}-biphenyl-4-ol (87 mg, 0.2 mmol) was treated as described in general procedure I using methyl 5-fluoro-2-(trifluoromethyl)benzoate (67 mg, 0.3 mmol) to give 5-(4′-{2-[4-(2,4-dichloro-phenyl)-1-ethyl-1H-imidazol-2-yl]-(E)-vinyl}-biphenyl-4-yloxy)-2-trifluoromethyl-benzoic acid methyl ester, which was treated directly as described in general procedure F to give 5-(4′-{2-[4-(2,4-dichloro-phenyl)-1-ethyl-1H-imidazol-2-yl]-(E)-vinyl}-biphenyl-4-yloxy)-2-trifluoromethyl-benzoic acid (76 mg, 61%).

LCMS: m/z 623 (M+H)⁺; ¹H NMR (DMSO-d₆, 400 MHz): δ 1.38 (t, 3H), 4.26 (q, 2H), 7.20 (d, 2H), 7.23 (d, 2H), 7.29 (d, 2H), 7.37 (d, 1H), 7.44 (dd, 1H), 7.57 (d, 1H), 7.61 (d, 1H), 7.65 (d, 2H), 7.72-7.78 (m, 3H), 7.85 (s, 1H), 8.15 (d, 1H) ppm.

### Example 197

4′-{2-[4-(2,4-Dichloro-phenyl)-1-ethyl-1H-imidazol-2-yl]-(E)-vinyl}-biphenyl-4-ol (87 mg, 0.2 mmol) was treated as described in general procedure I using methyl 5-fluoro-2-nitrobenzoate (60 mg, 0.3 mmol) to give 5-(4′-{2-[4-(2,4-dichloro-phenyl)-1-ethyl-1H-imidazol-2-yl]-(E)-vinyl}-biphenyl-4-yloxy)-2-nitro-benzoic acid methyl ester. The nitro-derivative was then treated as described in general procedure K to give 2-amino-5-(4′-{2-[4-(2,4-dichloro-phenyl)-1-ethyl-1H-imidazol-2-yl]-(E)-vinyl}-biphenyl-4-yloxy)-benzoic acid methyl ester, which was hydrolyzed as described in general procedure F to give 2-amino-5-(4′-{2-[4-(2,4-dichloro-phenyl)-1-ethyl-1H-imidazol-2-yl]-(E)-vinyl}-biphenyl-4-yloxy)-benzoic acid (56 mg, 49%).

LCMS: m/z 570 (M+H)⁺.

### Example 198

4-Bromophenylacetic acid (215 mg, 1 mmol) was treated according to general procedure A using 2,4-dichlorophenacyl bromide to give 2-(4-bromo-benzyl)-4-(2,4-dichloro-phenyl)-1H-imidazole, which was treated as described in general procedure E using 1-fluoro-4-nitrobenzene as the aryl halide and Cs₂CO₃ as the base to give 2-(4-bromo-benzyl)-4-(2,4-dichloro-phenyl)-1-(4-nitro-phenyl)-1H-imidazole. The bromo-nitro derivative was treated as described in general procedure B using 3-(methylsulfonylphenyl)boronic acid to give 4-(2,4-dichloro-phenyl)-2-(3′-methanesulfonyl-biphenyl-4-ylmethyl)-1-(4-nitro-phenyl)-1H-imidazole, which was then treated as described in general procedure Y (using methyl bromoacetate in Y2) to give 5-{4-[4-(2,4-dichloro-phenyl)-2-(3′-methanesulfonyl-biphenyl-4-ylmethyl)-imidazol-1-yl]-phenyl}-1,2,5-thiadiazolidine-3-one-1,1-dioxide (20 mg, 3%).

LCMS: m/z 667 (M+H)⁺; ¹H NMR (DMSO-d₆, 400 MHz): δ 3.28 (s, 3H), 3.87 (s, 2H), 4.26 (s, 2H), 7.16 (d, 2H), 7.24 (d, 2H), 7.32 (d, 2H), 7.67-7.98 (m, 9H), 8.13 (d, 1H) ppm.

### Example 199

4-Bromophenylacetic acid (107.5 g, 0.5 mol) was treated according to general procedure A using 2,4-dichlorophenacyl bromide to give 2-(4-bromo-benzyl)-4-(2,4-dichloro-phenyl)-1H-imidazole, which was treated as described in general procedure E using bromoethane to give 2-(4-bromo-benzyl)-4-(2,4-dichloro-phenyl)-1-ethyl-1H-imidazole. The bromo-derivative was treated as described in general procedure B using 4-methoxyphenylboronic acid to give 4-(2,4-dichloro-phenyl)-1-ethyl-2-(4′-methoxy-biphenyl-4-ylmethyl)-1H-imidazole, which was finally treated according to general procedure C to give 4′-[4-(2,4-dichloro-phenyl)-1-ethyl-1H-imidazol-2-ylmethyl]-biphenyl-4-ol (14.8 g, 7%). LCMS: m/z 423 (M+H)⁺.

4′-[4-(2,4-Dichloro-phenyl)-1-ethyl-1H-imidazol-2-ylmethyl]-biphenyl-4-ol (2.1 g, 5 mmol) was treated as described in general procedure J using methyl 2-amino-5-bromobenzoate (1.7 g, 7.5 mmol) to give 2-amino-5-{4′-[4-(2,4-dichloro-phenyl)-1-ethyl-1H-imidazol-2-ylmethyl]-biphenyl-4-yloxy}-benzoic acid methyl ester (629 mg, 22%).

LCMS: m/z 572 (M+H)⁺.

2-Amino-5-{4′-[4-(2,4-dichloro-phenyl)-1-ethyl-1H-imidazol-2-ylmethyl]-biphenyl-4-yloxy}-benzoic acid methyl ester (17 mg, 0.03 mmol) was treated as described in general procedure F to give 2-amino-5-{4′-[4-(2,4-dichloro-phenyl)-1-ethyl-1H-imidazol-2-ylmethyl]-biphenyl-4-yloxy}-benzoic acid (14 mg, 84%).

LCMS: m/z 558 (M+H)⁺; ¹H NMR (DMSO-d₆, 400 MHz): δ 1.18 (t, 3H), 3.97 (q, 2H), 4.16 (s, 2H), 7.04 (d, 2H), 7.21 (dd, 1H), 7.31 (d, 2H), 7.42 (dd, 1H), 7.48 (d, 1H), 7.56-7.60 (m, 4H), 7.62 (d, 2H), 7.84 (s, 1H), 8.16 (d, 1H) ppm.

### Example 200

4′-[4-(2,4-Dichloro-phenyl)-1-ethyl-1H-imidazol-2-ylmethyl]-biphenyl-4-ol (42 mg, 0.1 mmol) was treated as described in general procedure I using methyl 5-fluoro-2-nitrobenzoate (30 mg, 0.15 mmol) to give 5-{4′-[4-(2,4-dichloro-phenyl)-1-ethyl-1H-imidazol-2-ylmethyl]-biphenyl-4-yloxy}-2-nitro-benzoic acid methyl ester (47 mg, 78%).

LCMS: m/z 602 (M+H)⁺.

5-{4′-[4-(2,4-Dichloro-phenyl)-1-ethyl-1H-imidazol-2-ylmethyl]-biphenyl-4-yloxy}-2-nitro-benzoic acid methyl ester (18 mg, 0.03 mmol) was treated as described in general procedure F to give 5-{4′-[4-(2,4-dichloro-phenyl)-1-ethyl-1H-imidazol-2-ylmethyl]-biphenyl-4-yloxy}-2-nitro-benzoic acid (15 mg, 86%).

LCMS: m/z 588 (M+H)⁺; ¹H NMR (DMSO-d₆, 400 MHz): δ 1.19 (t, 3H), 3.98 (q, 2H), 4.17 (s, 2H), 7.05 (d, 2H), 7.22 (dd, 1H), 7.32 (d, 2H), 7.43 (dd, 1H), 7.49 (d, 1H), 7.56-7.61 (m, 4H), 7.63 (d, 2H), 7.84 (s, 1H), 8.17 (d, 1H) ppm.

### Example 201

2-Amino-5-{4′-[4-(2,4-dichloro-phenyl)-1-ethyl-1H-imidazol-2-ylmethyl]-biphenyl-4-yloxy}-benzoic acid methyl ester (57 mg, 0.1 mmol) was treated as described in general procedure L using methane sulfonyl chloride (16 μL, 0.2 mmol) and DIEA (26 μL, 0.15 mmol). The resulted mixture of di-sulfonamide and mono-sulfonamide was concentrated and treated directly as described in general procedure F to give 5-{4′-[4-(2,4-dichloro-phenyl)-1-ethyl-1H-imidazol-2-ylmethyl]-biphenyl-4-yloxy}-2-methanesulfonylamino-benzoic acid (23 mg, 36%).

LCMS: m/z 636 (M+H)⁺; ¹H NMR (DMSO-d₆, 400 MHz): δ 1.19 (t, 3H), 2.95 (3, 3H), 3.97 (q, 2H), 4.17 (s, 2H), 7.04 (d, 2H), 7.18 (d, 1H), 7.32 (d, 2H), 7.43 (dd, 1H), 7.49 (d, 1H), 7.59-7.61 (m, 4H), 7.64 (d, 2H), 7.84 (s, 1H), 8.17 (d, 1H) ppm.

### Example 202

5-{4′-[4-(2,4-dichloro-phenyl)-1-ethyl-1H-imidazol-2-ylmethyl]-biphenyl-4-yloxy}-2-trifluoromethanesulfonylamino-benzoic acid was prepared by analagous methods to those used to prepare Example 202. LCMS: m/z 690 (M+H)⁺.

### Example 203

4′-[4-(2,4-Dichloro-phenyl)-1-ethyl-1H-imidazol-2-ylmethyl]-biphenyl-4-ol (85 mg, 0.2 mmol) was treated as described in general procedure I using methyl 5-fluoro-2-(trifluoromethyl)benzoate (67 mg, 0.3 mmol) to give 5-{4′-[4-(2,4-dichloro-phenyl)-1-ethyl-1H-imidazol-2-ylmethyl]-biphenyl-4-yloxy}-2-trifluoromethyl-benzoic acid methyl ester, which was treated directly as described in general procedure F to give 5-{4′-[4-(2,4-dichloro-phenyl)-1-ethyl-1H-imidazol-2-ylmethyl]-biphenyl-4-yloxy}-2-trifluoromethyl-benzoic acid (74 mg, 61%).

LCMS: m/z 611 (M+H)⁺; ¹H NMR (DMSO-d₆, 400 MHz): δ 1.19 (t, 3H), 3.98 (q, 2H), 4.19 (s, 2H), 7.20 (d, 2H), 7.23 (d, 2H), 7.35 (d, 2H), 7.44 (dd, 1H), 7.61 (d, 1H), 7.65 (d, 2H), 7.72-7.78 (m, 3H), 7.85 (s, 1H), 8.17 (d, 1H) ppm.

### Example 204

5-{4′-[4-(2,4-dichloro-phenyl)-1-ethyl-1H-imidazol-2-ylmethyl]-biphenyl-4-yloxy}-2-trifluoromethyl-benzoic acid (61 mg, 0.1 mmol) was treated as described in general procedure G using methanesulfonamide (12 mg, 0.12 mmol) and fluoro-N,N,N′,N′-tetramethylformamidinium hexafluorophosphate (TFFH, 53 mg, 0.2 mmol) in 1 mL THF to give N-(5-{4′-[4-(2,4-dichloro-phenyl)-1-ethyl-1H-imidazol-2-ylmethyl]-biphenyl-4-yloxy}-2-trifluoromethyl-benzoyl)-methanesulfonamide (22 mg, 32%).

LCMS: m/z 688 (M+H)⁺; ¹H NMR (DMSO-d₆, 400 MHz): δ 1.18 (t, 3H), 3.19 (s, 3H), 3.97 (q, 2H), 4.18 (s, 2H), 7.20 (d, 2H), 7.23 (d, 2H), 7.35 (d, 2H), 7.44 (dd, 1H), 7.61 (d, 1H), 7.65 (d, 2H), 7.72-7.78 (m, 3H), 7.85 (s, 1H), 8.17 (d, 1H) ppm.

### Example 205

4-{4′-[4-(2,4-dichloro-phenyl)-1-ethyl-1H-imidazol-2-ylmethyl]-biphenyl-4-yloxy}-2-trifluoromethyl-benzoic acid was prepared by analagous methods to those used to prepare Example 203. LCMS: m/z 611 (M+H)⁺.

### Example 206

N-(4-{4′-[4-(2,4-dichloro-phenyl)-1-ethyl-1H-imidazol-2-ylmethyl]-biphenyl-4-yloxy}-2-trifluoromethyl-benzoyl)-methanesulfonamide was prepared by analagous methods to those used to prepare Example 204. LCMS: m/z 688 (M+H)⁺.

### Example 207

4′-[4-(2,4-Dichloro-phenyl)-1-ethyl-1H-imidazol-2-ylmethyl]-biphenyl-4-ol (423 mg, 1 mmol) was treated as described in general procedure I using methyl 4-fluoro-2-nitrobenzoate (300 mg, 1.5 mmol) to give 4-{4′-[4-(2,4-dichloro-phenyl)-1-ethyl-1H-imidazol-2-ylmethyl]-biphenyl-4-yloxy}-2-nitro-benzoic acid methyl ester, which was then treated as described in general procedure K to give 2-amino-4-{4′-[4-(2,4-dichloro-phenyl)-1-ethyl-1H-imidazol-2-ylmethyl]-biphenyl-4-yloxy}-benzoic acid methyl ester (297 mg, 52% yield). LCMS: m/z 572 (M+H)⁺.

2-Amino-4-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using trifluoromethanesulfonic anhydride (68 μL, 0.4 mmol) and DIEA (53 μL, 0.3 mmol). The resulted mixture of di-sulfonamide and mono-sulfonamide was concentrated and treated directly as described in general procedure F to give 4-{4′-[4-(2,4-dichloro-phenyl)-1-ethyl-1H-imidazol-2-ylmethyl]-biphenyl-4-yloxy}-2-trifluoro-methanesulfonylamino-benzoic acid (36 mg, 26%).

LCMS: m/z 690 (M+H)⁺; ¹H NMR (DMSO-d₆, 400 MHz): δ 1.19 (t, 3H), 3.97 (q, 2H), 4.18 (s, 2H), 6.56 (dd, 1H), 7.14 (d, 2H), 7.23 (d, 1H), 7.34 (d, 2H), 7.44 (dd, 1H), 7.61 (d, 1H), 7.63 (d, 2H), 7.68 (d, 2H), 7.85 (s, 1H), 7.92 (d, 1H), 8.18 (d, 1H) ppm.

By analagous methods to those used to prepare Example 207, the following compounds were synthesized:

| Example | Name | LC/MS (m/z) |
|---|---|---|
| 208 | 3-{4′-[4-(2,4-dichloro-phenyl)-1-ethyl-1H- | 690 (M + H)⁺ |
| | imidazol-2-ylmethyl]-biphenyl-4-yloxy}- | |
| | 5-trifluoromethanesulfonylamino-benzoic | |
| | acid | |
| 209 | 4-{4′-[4-(2,4-dichloro-phenyl)-1-ethyl-1H- | 636 (M + H)⁺ |
| | imidazol-2-ylmethyl]-biphenyl-4-yloxy}- | |
| | 2-methane-sulfonylamino-benzoic acid | |
| 210 | 4-{4′-[4-(2,4-dichloro-phenyl)-1-ethyl-1H- | 690 (M + H)⁺ |
| | imidazol-2-ylmethyl]-biphenyl-4-yloxy}- | |
| | 3-trifluoromethanesulfonylamino-benzoic | |
| | acid | |
| 211 | 4-{4′-[4-(2,4-dichloro-phenyl)-1-ethyl-1H- | 636 (M + H)⁺ |
| | imidazol-2-ylmethyl]-biphenyl-4-yloxy}- | |
| | 3-methanesulfonylamino-benzoic acid | |
| 212 | 3-benzenesulfonylamino-4-{4′-[4-(2,4- | 698 (M + H)⁺ |
| | dichloro-phenyl)-1-ethyl-1H-imidazol-2- | |
| | ylmethyl]-biphenyl-4-yloxy}-benzoic acid | |

### Example 213

3-Amino-4-{4′-[4-(2,4-dichloro-phenyl)-1-ethyl-1H-imidazol-2-ylmethyl]-biphenyl-4-yloxy}-benzoic acid methyl ester (17 mg, 0.03 mmol) was treated as described in general procedure F to give 3-amino-4-{4′-[4-(2,4-dichloro-phenyl)-1-ethyl-1H-imidazol-2-ylmethyl]-biphenyl-4-yloxy}-benzoic acid (14 mg, 84%).

LCMS: m/z 558 (M+H)⁺; ¹H NMR (DMSO-d₆, 400 MHz): δ 1.18 (t, 3H), 3.97 (q, 2H), 4.16 (s, 2H), 7.04 (d, 2H), 7.21 (dd, 1H), 7.31 (d, 2H), 7.42 (dd, 1H), 7.48 (d, 1H), 7.56-7.60 (m, 4H), 7.62 (d, 2H), 7.84 (s, 1H), 8.16 (d, 1H) ppm.

By analagous methods to those used to prepare Example 207, the following compounds were synthesized:

| Example | Name | LC/MS (m/z) |
|---|---|---|
| 214 | 4-{4′-[4-(2,4-dichloro-phenyl)-1-ethyl-1H- | 712 (M + H)⁺ |
| | imidazol-2-ylmethyl]-biphenyl-4-yloxy}- | |
| | 3-phenylmethanesulfonylamino-benzoic | |
| | acid | |
| 215 | 4-{4′-[4-(2,4-dichloro-phenyl)-1-ethyl-1H- | 726 (M + H)⁺ |
| | imidazol-2-ylmethyl]-biphenyl-4-yloxy}- | |
| | 3-(2-phenyl-ethanesulfonylamino)-benzoic | |
| | acid | |
| 216 | 4-{4′-[4-(2,4-dichloro-phenyl)-1-ethyl-1H- | 766 (M + H)⁺ |
| | imidazol-2-ylmethyl]-biphenyl-4-yloxy}- | |
| | 3-(3-trifluoromethyl-benzenesulfonylamino)- | |
| | benzoic acid | |
| 217 | 4-{4′-[4-(2,4-dichloro-phenyl)-1-ethyl-1H- | 699 (M + H)⁺ |
| | imidazol-2-ylmethyl]-biphenyl-4-yloxy}- | |
| | 3-(pyridine-3-sulfonylamino)-benzoic acid | |

### Example 218

4′-[4-(2,4-Dichloro-phenyl)-1-ethyl-1H-imidazol-2-ylmethyl]-biphenyl-4-ol (85 mg, 0.2 mmol) was treated as described in general procedure I using methyl 6-chloronicotinate (52 mg, 0.3 mmol) to give 6-{4′-[4-(2,4-dichloro-phenyl)-1-ethyl-1H-imidazol-2-ylmethyl]-biphenyl-4-yloxy}-nicotinic acid methyl ester, which was hydrolyzed as described in general procedure F to give 6-{4′-[4-(2,4-dichloro-phenyl)-1-ethyl-1H-imidazol-2-ylmethyl]-biphenyl-4-yloxy}-nicotinic acid (35 mg, 32%).

LCMS: m/z 544 (M+H)⁺; ¹H NMR (DMSO-d₆, 400 MHz): δ 1.18 (t, 3H), 3.97 (q, 2H), 4.16 (s, 2H), 7.21 (d, 2H), 7.23 (d, 2H), 7.35 (d, 2H), 7.44 (dd, 1H), 7.61 (d, 1H), 7.61-7.73 (m, 5H), 7.85 (s, 1H), 8.15 (d, 1H) ppm.

By analagous methods to those used to prepare Example 207, the following compounds were synthesized:

| Example | Name | LC/MS (m/z) |
|---|---|---|
| 219 | 5-{4′-[4-(2,4-dichloro-phenyl)-1-ethyl-1H- | 704 (M + H)⁺ |
| | imidazol-2-ylmethyl]-biphenyl-4- | |
| | yloxy}-2-(2,2,2-trifluoro- | |
| | ethanesulfonylamino)-benzoic acid | |
| 220 | 5-{4′-[4-(2,4-dichloro-phenyl)-1-ethyl-1H- | 650 (M + H)⁺ |
| | imidazol-2-ylmethyl]-biphenyl-4-yloxy}-2- | |
| | ethanesulfonylamino-benzoic acid | |

### Example 221

5-{4′-[4-(2,4-Dichloro-phenyl)-1-ethyl-1H-imidazol-2-ylmethyl]-biphenyl-4-yloxy}-2-methanesulfonylamino-benzoic acid (64 mg, 0.1 mmol) was treated as described in general procedure P using iodomethane (13 μL, 0.2 mmol). The resulted mixture was concentrated and treated as described in general procedure F to give 5-{4′-[4-(2,4-dichloro-phenyl)-1-ethyl-1H-imidazol-2-ylmethyl]-biphenyl-4-yloxy}-2-(methanesulfonyl-methyl-amino)-benzoic acid (15 mg, 23%).

LCMS: m/z 650 (M+H)⁺; ¹H NMR (DMSO-d₆, 400 MHz): δ 1.19 (t, 3H), 2.95 (s, 3H), 3.44 (s, 3H), 3.97 (q, 2H), 4.17 (s, 2H), 6.76 (dd, 1H), 7.14 (d, 2H), 7.23 (d, 1H), 7.34 (d, 2H), 7.44 (dd, 1H), 7.62-7.72 (m, 5H), 7.84 (s, 1H), 7.92 (d, 1H), 8.13 (d, 1H) ppm.

### Example 222

5-{4′-[4-(2,4-dichloro-phenyl)-1-ethyl-1H-imidazol-2-ylmethyl]-biphenyl-4-yloxy}-2-(methyl-trifluoromethane sulfonyl-amino)-benzoic acid was prepared by analagous methods to those used to prepare Example 221. LCMS: m/z 704 (M+H)⁺.

### Example 223

2-(4-Bromo-benzyl)-4-(2,4-dichloro-phenyl)-1-ethyl-1H-imidazole (205 mg, 0.5 m mol) was treated as described in general procedure B using 3-methoxyphenylboronic acid (152 mg, 1 mmol) to give 4-(2,4-dichloro-phenyl)-1-ethyl-2-(3′-methoxy-biphenyl-4-ylmethyl)-1H-imidazole, which was treated as described in general procedure C to give 4′-[4-(2,4-dichloro-phenyl)-1-ethyl-1H-imidazol-2-ylmethyl]-biphenyl-3-ol. The phenol was treated as described in general procedure I using 5-fluoro-2-(trifluoromethyl)benzoic acid methyl ester (222 mg, 1 mmol) to give 5-{4′-[4-(2,4-dichloro-phenyl)-1-ethyl-1H-imidazol-2-ylmethyl]-biphenyl-3-yloxy}-2-trifluoromethyl-benzoic acid methyl ester. The ester was hydrolyzed as described in general procedure F to give 5-{4′-[4-(2,4-dichloro-phenyl)-1-ethyl-1H-imidazol-2-ylmethyl]-biphenyl-3-yloxy}-2-trifluoromethyl-benzoic acid (52 mg, 17%).

LCMS: m/z 611 (M+H)⁺; ¹H NMR (DMSO-d₆, 400 MHz): δ 1.18 (t, 3H), 3.97 (q, 2H), 4.17 (s, 2H), 7.21 (d, 2H), 7.23 (d, 2H), 7.35 (d, 2H), 7.44 (dd, 1H), 7.61 (d, 1H), 7.65-7.78 (m, 5H), 7.85 (s, 1H), 8.15 (d, 1H) ppm.

### Example 224

4-[2-(4-Bromo-benzyl)-4-(2,4-dichloro-phenyl)-imidazol-1-ylmethyl]-benzoic acid methyl ester (265 mg, 0.5 mmol) was treated as described in general procedure B using 4-hydroxyphenylboronic acid (86 mg, 0.6 mmol) to give 4-[4-(2,4-dichloro-phenyl)-2-(4′-hydroxy-biphenyl-4-ylmethyl)-imidazol-1-ylmethyl]-benzoic acid methyl ester (210 mg, 77%).

The resulted 4-[4-(2,4-dichloro-phenyl)-2-(4′-hydroxy-biphenyl-4-ylmethyl)-imidazol-1-ylmethyl]-benzoic acid methyl ester (200 mg, 0.37 mmol) was treated using 4-tert-butyl-benzeneboronic acid (98 mg, 0.55 mmol) according to general procedure W to give 4-[2-[4′-(4-tert-butyl-phenoxy)-biphenyl-4-ylmethyl]-4-(2,4-dichloro-phenyl)-imidazol-1-ylmethyl]-benzoicacid methyl ester (136 mg, 54%).

4-[2-[4′-(4-tert-Butyl-phenoxy)-biphenyl-4-ylmethyl]-4-(2,4-dichloro-phenyl)-imidazol-1-ylmethyl]-benzoic acid (48 mg, 73% yield) was prepared according to general procedure F using 4-[2-[4′-(4-tert-Butyl-phenoxy)-biphenyl-4-ylmethyl]-4-(2,4-dichloro-phenyl)-imidazol-1-ylmethyl]-benzoic acid methyl ester (68 mg, 0.1 mmol).

LCMS: m/z 662 (M+H)⁺; ¹H NMR (DMSO-d₆, 400 MHz): δ 1.28 (s, 9H), 4.10 (s, 2H), 5.35 (s, 2H), 6.97 (dd, 2H), 7.01 (d, 2H), 7.16 (d, 2H), 7.22 (d, 2H), 7.40-7.47 (m, 4H), 7.56-7.62 (m, 4H), 7.82 (d, 2H), 7.95 (s, 1H), 8.18 (d, 1H) ppm.

### Example 225

4-[4-(2,4-Dichloro-phenyl)-2-(4′-hydroxy-biphenyl-4-ylmethyl)-imidazol-1-ylmethyl]-benzoic acid methyl ester (200 mg, 0.37 mmol) was treated with 1-bromo 4, 4, 4, trifluoro butane (89 mg, 0.47 mmol) according to general procedure E to 4-{4-(2,4-dichloro-phenyl)-2-[4′-(4,4,4-trifluoro-butoxy)-biphenyl-4-ylmethyl]-imidazol-1-ylmethyl}-benzoic acid methyl ester (186 mg, 78%).

4-{4-(2,4-Dichloro-phenyl)-2-[4′-(4,4,4-trifluoro-butoxy)-biphenyl-4-ylmethyl]-imidazol-1-ylmethyl}-benzoic acid (49 mg, 76% yield) was prepared according to general procedure F using 4-{4-(2,4-dichloro-phenyl)-2-[4′-(4,4,4-trifluoro-butoxy)-biphenyl-4-ylmethyl]-imidazol-1-ylmethyl}-benzoic acid methyl ester (66 mg, 0.1 mmol).

LCMS: m/z 662 (M+H)⁺; ¹H NMR (DMSO-d₆, 400 MHz): δ 1.90 (q, 2H), 1.96 (q, 2H), 2.39-2.46 (m, 2H), 4.09 (s, 2H), 5.31 (s, 2H), 6.98 (d, 2H), 7.01 (d, 2H), 7.14 (d, 2H), 7.22 (d, 2H), 7.43-7.47 (m, 2H), 7.51 (d, 1H), 7.53 (d, 2H), 7.82 (d, 1H), 7.93 (s, 1H), 8.17 (d, 1H) ppm.

### Example 226

4-[2-(4-Bromo-benzyl)-4-(2,4-dichloro-phenyl)-imidazol-1-ylmethyl]-benzoic acid methyl ester (265 mg, 0.5 mmol) was treated as described in general procedure B using 3-amino phenylboronic acid (86 mg, 0.62 mmol) to give 4-[4-(2,4-dichloro-phenyl)-2-(3′-amino-biphenyl-4-ylmethyl)-imidazol-1-ylmethyl]-benzoic acid methyl ester (204 mg, 75%).

4-[4-(2,4-Dichloro-phenyl)-2-(3′-amino-biphenyl-4-ylmethyl)-imidazol-1-ylmethyl]-benzoic acid methyl ester (200 mg, 0.37 mmol) was treated with 2,2,2-trifluoroethane sulfonylchloride (67 mg, 0.36 mmol) according to general procedure L to give 4-{4-(2,4-dichloro-phenyl)-2-[3′-(2,2,2-trifluoro-ethanesulfonylamino)-biphenyl-4-ylmethyl]-imidazol-1-ylmethyl}-benzoic acid methyl ester (146 mg, 58%).

4-{4-(2,4-Dichloro-phenyl)-2-[3′-(2,2 ,2-trifluoro-ethanesulfonylamino)-biphenyl-4-ylmethyl]-imidazol-1-ylmethyl}-benzoic acid (52 mg, 75% yield) was prepared according to general procedure F using 4-{4-(2,4-dichloro-phenyl)-2-[3′-(2,2,2-trifluoro-ethanesulfonylamino)-biphenyl-4-ylmethyl]-imidazol-1-ylmethyl}-benzoic acid methyl ester (69 mg, 0.1 mmol).

LCMS: m/z 675 (M+H)⁺; ¹H NMR (DMSO-d₆, 400 MHz): δ 4.19 (s, 2H), 4.58 (s, 2H), 5.40 (s, 2H), 7.19 (d, 2H), 7.21 (d, 2H), 7.27 (d, 1H), 7.29-7.35 (m, 2H), 7.39 (d. 1H), 7.41 (d, 2H), 7.47-7.49 (m, 2H), 7.66 (s, 1H), 7.84 (d, 2H), 8.03 (s, 1H), 10.5 (s, 1H) ppm.

### Example 227

4-[2-(4-Bromo-benzyl)-4-(2,4-dichloro-phenyl)-imidazol-1-ylmethyl]-benzoic acid methyl ester (265 mg, 0.5 mmol) was treated as described in general procedure B using 4-N-Boc-amino-3-methoxy phenylboronic acid (200 mg, 0.74 mmol) to give 4-[2-(4′-tert-butoxycarbonylamino-3′-methoxy-biphenyl-4-ylmethyl)-4-(2,4-dichloro-phenyl)-imidazol-1-ylmethyl]-benzoic acid methyl ester (228 mg, 68%).

4-[2-(4′-tert-Butoxycarbonylamino-3′-methoxy-biphenyl-4-ylmethyl)-4-(2,4-dichloro-phenyl)-imidazol-1-ylmethyl]-benzoic acid (51 mg, 77% yield) was prepared according to general procedure F using 4-[2-(4′-tert-butoxycarbonylamino-3′-methoxy-biphenyl-4-ylmethyl)-4-(2,4-dichloro-phenyl)-imidazol-1-ylmethyl]-benzoic acid methyl ester (68 mg, 0.1 mmol).

LCMS: m/z 659 (M+H)⁺; ¹H NMR (DMSO-d₆, 400 MHz): δ 1.46 (s, 9H), 3.87 (s, 3H), 4.10 (s, 2H), 5.35 (s, 2H), 7.10 (d, 1H), 7.14 (d, 2H), 7.20 (d, 2H), 7.45 (d, 2H), 7.49 (d, 2H), 7.51 (s, 1H), 7.62 (d, 2H), 7.73 (d, 2H), 7.83 (s, 1H), 8.18 (d, 1H) ppm.

### Example 228

4-[4-(2,4-Dichloro-phenyl)-2-(4′-isopropoxycarbonylamino-3′-methoxy-biphenyl-4-ylmethyl)-imidazol-1-ylmethyl]-benzoic acid was prepared by analagous methods to those used to prepare Example 227 .

LCMS: m/z 645 (M+H)⁺; ¹H NMR (DMSO-d₆, 400 MHz): δ 1.23 (d, 6H), 3.87 (s, 3H), 4.12 (s, 2H), 4.89 (q, 1H), 5.46 (s, 2H), 7.13-7.16 (m, 4H), 7.18 (d, 2H), 7.24 (d, 2H), 7.51 (d, 2H), 7.73 (d, 2H), 7.82 (d, 2H), 8.27 (s, 1H) ppm.

### Example 229

N-{4-[2-[4′-(4-tert-Butyl-phenoxy)-biphenyl-4-ylmethyl]-4-(2,4-dichloro-phenyl)-imidazol-1-ylmethyl]-benzoyl}-methanesulfonamide (19 mg, 51%) was prepared from methanesulfonamide (5 mg, 0.045 mmol) and 4-[2-[4′-(4-tert-butyl-phenoxy)-biphenyl-4-ylmethyl]-4-(2,4-dichloro-phenyl)-imidazol-1-ylmethyl]-benzoic acid (33 mg, 0.05 mmol) according to the general procedure AA.

LCMS: m/z 739 (M+H)⁺; ¹H NMR (DMSO-d₆, 400 MHz): δ 1.27 (s, 9H), 3.47 (s, 3H), 4.12 (s, 2H), 5.36 (s, 2H), 6.96 (dd, 2H), 7.02 (d, 2H), 7.14 (d, 2H), 7.25 (d, 2H), 7.40-7.47 (m, 4H), 7.53-7.61 (m, 4H), 7.80 (d, 2H), 7.85 (s, 1H), 8.20 (d, 1H) ppm.

### Example 230

2-(4-Bromo-benzyl)-4-(2,4-dichloro-phenyl)-1H-imidazole (19.1 g, 50 mmol) was treated as described in general procedure E using 4-nitro-benzyl bromide to give 4-[2-(4-Bromo-benzyl)-4-(2,4-dichloro-phenyl)-nitro benzyl imidazol (17.5 g, 67%).

4-[2-(4-Bromo-benzyl)-4-(2,4-dichloro-phenyl)-nitro benzyl imidazole (5.2 g, 10 mmol) was treated as described in general procedure B using 3-(methyl sulfonylamino)-phenylboronic acid (2.8 g, 13 mmol) to give N-{4′-[1-(4-Nitro-benzyl)-4-(2,4-dichloro-phenyl)-1H-imidazol-2-ylmethyl]-biphenyl-3-yl} methanesulfonamide (3.9 g, 64%).

LCMS: m/z 608 (M+H)⁺.

### Example 231

N-{4′-[4′-(4-Nitro-benzyl)-4-(2,4-dichloro-phenyl)-1H-imidazol-2-ylmethyl]-biphenyl-3-yl} methanesulfonamide (3.0 g 5.0 mmol) was reduced according to general procedure K to give N-{4′-[1-(4-Amino-benzyl)-4-(2,4-dichloro-phenyl)-1H-imidazol-2-ylmethyl]-biphenyl-3-yl}-methanesulfonamide (2.2 g 77%).

The resulted N-{4′-[4′-(4-Amino-benzyl)-4-(2,4-dichloro-phenyl)-1H-imidazol-2-ylmethyl]-biphenyl-3-yl}-methanesulfonamide (2.0 g, 3.5 mmol)) was treated with methyl bromoacetate (0.6 g, 3.9 mmol) according to general procedure E to give {4-[4-(2,4-dichloro-phenyl)-2-(3′-methanesulfonylamino-biphenyl-4-ylmethyl)-imidazol-1-ylmethyl]-phenylamino}-acetic acid methyl ester (1.76 g, 80%).

{4-[4-(2,4-Dichloro-phenyl)-2-(3′-methanesulfonylamino-biphenyl-4-ylmethyl)-imidazol-1-ylmethyl]-phenylamino}-acetic acid (51 mg, 77%) was prepared according to general procedure F using {4-[4-(2,4-Dichloro-phenyl)-2-(3′-methanesulfonylamino-biphenyl-4-ylmethyl)-imidazol-1-ylmethyl]-phenylamino}-acetic acid methyl ester (65 mg, 0.1 mmol).

LCMS: m/z 636 (M+H)⁺; ¹H NMR (DMSO-d₆, 400 MHz): δ 1.22 (s, 2H), 3.07 (s, 2H), 3.35 (s, 3H), 4.14 (s, 2H), 7.17 (d, 2H), 7.21-7.46 (m, 6H), 7.54 (d, 2H), 7.64 (d, 2H), 7.97 (d, 2H), 8.04 (d, 2H), 8.19 (s, 1H) ppm.

### Example 232

3-Trifluoromethylphenylacetic acid (10 g, 50 mol) was treated according to general procedure A using 2,4-dichlorophenacyl bromide to give 4-(2,4-Dichloro-phenyl)-2-(3-trifluoromethyl-benzyl)-1H-imidazole (8.2 g, 45% yield).

4-(2,4-Dichloro-phenyl)-2-(3-trifluoromethyl-benzyl)-1H-imidazole (3.8 g, 10 mmol) was treated as described in general procedure E using 4-nitro-benzyl bromide to give 4-[2-(3-trifluoro-benzyl)-4-(2,4-dichloro-phenyl)-nitro benzyl imidazol (3.5 g, 68% yield).

The resulted 4-[2-(3-trifluoro-benzyl)-4-(2,4-dichloro-phenyl)-nitro benzyl imidazol (2.5 g, 5 mmol) was reduced according to general procedure K and alkylated with methyl bromoacetate following general procedure E to give {4-[4-(2,4-Dichloro-phenyl)-2-(3-trifluoromethyl-benzyl)-imidazol-1-ylmethyl]-phenylamino}-acetic acid methyl ester (1.8 g, 66%).

5-{4-[4-(2,4-Dichloro-phenyl)-2-(3-trifluoromethyl-benzyl)-imidazol-1-ylmethyl]-phenyl}-1-[1,2,5]-thiadiazolidin-3-one-1,1-dioxide (28 mg, 55% yield) was prepared according to general procedure Y3 using {4-[4-(2,4-Dichloro-phenyl)-2-(3-trifluoromethyl-benzyl)-imidazol-1-ylmethyl]-phenylamino}-acetic acid methyl ester (55 mg, 0.1 mmol).

LCMS: m/z 696 (M+H)⁺; ¹H NMR (DMSO-d₆, 400 MHz): δ 3.93 (s, 2H), 4.23 (s, 2H), 5.17 (s, 2H), 6.90 (d, 1H), 7.01 (d, 1H), 7.10 (d, 2H), 7.41 (d, 2H), 7.49 (d, 2H), 7.59 (d, 2H), 7.88 (s, 1H), 8.10 (d, 1H) ppm.

### Example 233

4-{4-(2,4-Dichloro-phenyl)-2-[2-(4′-trifluoromethyl-biphenyl-4-yl)-ethyl]-imidazol-1-ylmethyl}-benzoic acid (30 mg, 50%) was prepared according to general procedure D from 4-{4-(2,4-Dichloro-phenyl)-2-[2-(4′-trifluoromethyl-biphenyl-4-yl)-(E)-vinyl]-imidazol-1-ylmethyl}-benzoic acid (59 mg, 0.1 mmol).

LCMS: m/z 596 (M+H)⁺; ¹H NMR (DMSO-d₆, 400 MHz): δ 2.91-2.98 (m, 2H), 3.07-3.38 (m, 2H), 5.35 (s, 2H), 7.17 (d, 2H), 7.25 (d, 2H), 7.31 (d, 2H), 7.37 (dd, 1H), 7.47 (d, 2H), 7.54 (d, 2H), 7.58-8.02 (m, 4H), 8.10 (d, 1H) ppm.

### Example 234

4-{4-(2,4-Dichloro-phenyl)-2-[2-(3′-trifluoromethyl-biphenyl-4-yl)-ethyl]-imidazol-1-ylmethyl}-benzoic acid (28 mg, 48%) was prepared was prepared by analagous methods to those used to prepare Example 233.

LCMS: m/z 596 (M+H)⁺.

### Example 235

4-{4-(2,4-Dichloro-phenyl)-2-[2-(3′-methanesulfonyl-biphenyl-4-yl)-(E)-vinyl]-imidazol-1-ylmethyl}-benzoic acid (60 mg, 0.1 mmol) was reduced following general procedure D to give 4-{4-(2,4-dichloro-phenyl)-2-[2-(3′-methanesulfonyl-biphenyl-4-yl)-ethyl]-imidazol-1-ylmethyl}-benzoic acid (29 mg, 48%).

LCMS: m/z 606 (M+H)⁺; ¹H NMR (DMSO-d₆, 400 MHz): δ 2.03 (m, 2H), 2.69 (m, 2H), 3.28 (s, 3H), 5.34 (s, 2H), 7.15 (d, 2H), 7.26 (d, 2H), 7.39 (d, 2H), 7.43 (dd, 1H), 7.68 (d, 2H), 7.74 (d, 2H), 7.85-8.02 (m, 4H), 8.15 (d, 1H) ppm.

### Example 236

4-Trifluoromethyl hydrocinnamic acid (11 g, 50 mol) was treated according to general procedure A using 2,4-dichlorophenacyl bromide to give 4-(2,4-Dichloro-phenyl)-2-[2-(4-trifluoromethyl-phenyl)-ethyl]-1H-imidazole (6.2 g, 33% yield).

4-(2,4-Dichloro-phenyl)-2-[2-(4-trifluoromethyl-phenyl)-ethyl]-1H-imidazole (3.8 g, 10 mmol) was treated as described in general procedure E using 4-nitro-benzyl bromide to give 4-{4-(2,4-Dichloro-phenyl)-2-[2-(4-trifluoromethyl-phenyl)-ethyl]-nitrobenzyl imidazole (2.8 g, 54%).

LCMS: m/z 521 (M+H)⁺.

### Example 237

4-{4-(2,4-Dichloro-phenyl)-2-[2-(4-trifluoromethyl-phenyl)-ethyl]-nitrobenzyl imidazole (2.6 g, 5 mmol) was reduced according to general procedure K and alkylated with methyl bromoacetate following general procedure E to give (4-{4-(2,4-dichloro-phenyl)-2-[2-(4-trifluoromethyl-phenyl)-ethyl]-imidazol-1-ylmethyl}-phenylamino)-acetic acid methyl ester (1.8 g, 64%).

5-(4-{4-(2,4-Dichloro-phenyl)-2-[2-(4-trifluoromethyl-phenyl)-ethy]-imidazol-1-ylmethyl}-phenyl)-1,2,5-thiadiazolidin-3-one 1,1dioxide (28 mg, 54% yield) was prepared according to general procedure Y3 from (4-{4-(2,4-Dichloro-phenyl)-2-[2-(4-trifluoromethyl-phenyl)-ethyl]-imidazol-1-ylmethyl}-phenylamino)-acetic acid methyl ester (56 mg, 0.1 mmol).

LCMS: m/z 610 (M+H)⁺; ¹H NMR (DMSO-d₆, 400 MHz): δ 2.96 (m, 2H), 3.03 (m, 2H), 3.95 (s, 2H), 5.15 (s, 2H), 7.03 (d, 2H), 7.12 (d, 2H), 7.43 (d, 1H), 7.45 (d, 2H), 7.59-7.63 (m, 2H), 7.87 (s, 1H), 8.15 (d, 2H) ppm.

### Example 238

5-(4-{4-(2,4-Dichloro-phenyl)-2-[2-(2-fluoro-4-trifluoromethyl-phenyl)-ethyl]-imidazol-1-ylmethyl}-phenyl)-1,2,5-thiadiazolidin-3-one-1,1-dioxide (63 mg, 0.1 mmol) was reduced following general procedure D to give 5-(4-{4-(2,4-Dichloro-phenyl)-2-[2-(2-fluoro-4-trifluoromethyl-phenyl)-ethy]-imidazol-1-ylmethyl}-phenyl)-1,2,5-thiadiazolidin-3-one-1,1-dioxide (29 mg, 46% yield).

LCMS: m/z 628 (M+H)⁺; ¹H NMR (DMSO-d₆, 400 MHz): δ 2.97 (m, 2H), 3.07 (m, 2H), 3.97 (s, 2H), 5.16 (s, 2H), 7.03 (d, 2H), 7.13 (d, 2H), 7.25 (d, 1H), 7.42 (d, 2H), 7.49-7.59 (m, 2H), 7.65 (d, 1H), 7.88 (s, 1H) ppm.

### Example 239

5-(4-{4-(2,4-Dichloro-phenyl)-2-[2-(3′-trifluoromethoxy-biphenyl-4-yl)-ethyl]-imidazol-1-ylmethyl}-phenyl)-1,2,5-thiadiazolidin-3-one-1,1-dioxide (69 mg, 0.1 mmol) was reduced following general procedure D to give 5-(4-{4-(2,4-dichloro-phenyl)-2-[2-(3′-trifluoromethoxy-biphenyl-4-yl)-ethyl]-imidazol-1-ylmethyl}-phenyl)-1,2,5-thiadiazolidin-3-one-1,1-dioxide (27 mg, 39% yield).

LCMS: m/z 702 (M+H)⁺; ¹H NMR (DMSO-d₆, 400 MHz): δ 2.96 (m, 2H), 3.01 (m, 2H), 3.92 (s, 2H), 5.15 (s, 2H), 7.03 (d, 1H), 7.05 (d, 1H), 7.13 (d, 2H), 7.26 (d, 2H), 7.36-7.48 (m, 2H), 7.50-7.60 (m, 2H), 7.63 (d, 2H), 7.75 (d, 2H), 7.88 (s, 1H), 8.18 (d, 1H), 8.24 (d, 1H) ppm.

### Example 240

4-Methoxybenzoic acid (7.5 g, 50 mol) was treated according to general procedure A using 2,4-dichlorophenacyl bromide to give 4-(2,4-dichloro-phenyl)-2-(4-methoxy-phenyl)-1H-imidazole (6.2 g, 39%).

4-(2,4-Dichloro-phenyl)-2-(4-methoxy-phenyl)-1H-imidazole (3.2 g, 10 mmol) was treated as described in general procedure E using methyl 4-bromomethyl benzoate (2.5 g, 11 mmol) to give 4-[4-(2,4-dichloro-phenyl)-2-(4-methoxy-phenyl)-imidazol-1-yl-methyl]-benzoic acid methyl ester (3.2 g, 68% yield).

4-[4-(2,4-Dichloro-phenyl)-2-(4-methoxy-phenyl)-imidazol-1-ylmethyl]-benzoic acid methyl ester (2.3 g, 5 mmol) was hydrolyzed following general procedure F to give 4-[4-(2,4-dichloro-phenyl)-2-(4-methoxy-phenyl)-imidazol-1-ylmethyl]-benzoic acid (1.67 g, 75%).

LCMS: m/z 454 (M+H)⁺.

### Example 241

4-[4-(2,4-Dichloro-phenyl)-2-(4-methoxy-phenyl)-imidazol-1-ylmethyl]-benzoic acid methyl ester (2.3 g, 5 mmol) was dealkylated according to general procedure C to give 4-[4-(2,4-dichloro-phenyl)-2-(4-hydroxy-phenyl)-imidazol-1-ylmethyl]-benzoic acid methyl ester (1.8 g, 78%).

4-[4-(2,4-Dichloro-phenyl)-2-(4-hydroxy-phenyl)-imidazol-1-ylmethyl]-benzoic acid methyl ester (1.5 g, 3.3 mmol) was alkylated with 4-methylsulfonyl benzy bromide (0.99 g, 3.9 mmol) following general procedure E to give 4-{4-(2,4-dichloro-phenyl)-2-[4-(4-methanesulfonyl-benzyloxy)-phenyl]-imidazol-1-ylmethyl}-benzoic acid methyl ester (1.4 g, 68%).

4-{4-(2,4-Dichloro-phenyl)-2-[4-(4-methanesulfonyl-benzyloxy)-pheny]-imidazol-1-ylmethyl}-benzoic acid methyl ester (310 mg, 0.5 mmol) was hydrolyzed following general procedure F to give 4-{4-(2,4-dichloro-phenyl)-2-[4-(4-methanesulfonyl-benzyloxy)-phenyl]-imidazol-1-ylmethyl}-benzoic acid (255 mg, 84%).

LCMS: m/z 608 (M+H)⁺; ¹H NMR (DMSO-d₆, 400 MHz): δ 3.22 (s, 3H), 5.28 (s, 2H), 5.49 (s, 2H), 7.08 (d, 2H), 7.10 (d, 2H), 7.53 (d, 2H), 7.64 (s, 1H), 7.70 (d, 2H), 7.72 (d, 2H), 7.94 (d, 2H), 8.08 (s, 1H), 8.23 (d, 2H) ppm.

### Example 242

4-[4-(2,4-Dichloro-phenyl)-2-(4-hydroxy-phenyl)-imidazol-1-ylmethyl]-benzoic acid methyl ester (1.5 g, 3.3 mmol) was treated with 3-methylsulfony phenylboronic acid (0.9 g, 4.9 mmol) as described in general procedure W to give 4-{4-(2,4-dichloro-phenyl)-2-[4-(3-methanesulfonyl-phenoxy)-pheny]-imidazol-1-ylmethyl}-benzoic acid methyl ester (1.2 g, 63%).

4-{4-(2,4-Dichloro-phenyl)-2-[4-(3-methanesulfonyl-phenoxy)-pheny]-imidazol-1-ylmethyl}-benzoic acid methyl ester (304 mg, 0.5 mmol) was hydrolyzed following general procedure F to give 4-{4-(2,4-dichloro-phenyl)-2-[4-(3-methanesulfonyl-phenoxy)-pheny]-imidazol-1-ylmethyl}-benzoic acid methyl ester (225 mg, 76%).

LCMS: m/z 594 (M+H)⁺; ¹H NMR (DMSO-d₆, 400 MHz): δ 3.25 (s, 3H), 5.54 (s, 2H), 7.14 (d, 2H), 7.17 (d, 1H), 7.38 (d, 2H), 7.47 (s, 1H), 7.50 (d, 2H), 7.65 (d, 2H), 7.66-7.7.88 (m, 2H), 7.90 (d, 2H), 8.14 (s, 1H), 8.24 (d, 1H) ppm.

### Example 243

4-(2,4-Dichloro-phenyl)-2-(4-methoxy-phenyl)-1H-imidazole (3.2 g, 10 mmol) was treated with 4-bromo benzyl bromide (3.0 g, 12 mmol) as described in general procedure E to give 1-(4-bromo-benzyl)-4-(2,4-dichloro-phenyl)-2-(4-methoxy-phenyl)-1H-imidazole (3.2 g, 66%).

1-(4-Bromo-benzyl)-4-(2,4-dichloro-phenyl)-2-(4-methoxy-phenyl)-1H-imidazole (2.4 g, 5 mmol) was treated as described in general procedure B using 3-trifluoromethyl phenylboronic acid (1.1 g, 5.8 mmol) to give 4-(2,4-dichloro-phenyl)-2-(4-methoxy-phenyl)-1-(3′-trifluoromethyl-biphenyl-4-ylmethyl) 1H-imidazole (2.1 mg, 77%).

LCMS: m/z 454 (M+H)⁺; ¹H NMR (DMSO-d₆, 400 MHz): δ 3.78 (s, 3H), 5.41 (s, 2H), 7.01 (d, 2H), 7.18 (d, 2H), 7.39 (d, 2H), 7.47-7.59 (m, 2H), 7.64 (d, 1H), 7.70 (d, 2H), 7.74 (d, 2H), 7.94 (s, 1H), 8.09 (s, 1H), 8.23 (d, 1H) ppm.

### Example 244

4-{4-[4-(2,4-Dichloro-phenyl)-1-(3′-trifluoromethyl-biphenyl-4-ylmethyl)-1H-imidazol-2-yl]-phenoxy}-butyric acid was prepared by analagous methods to those used to prepare Example 243. LCMS: m/z 626 (M+H)⁺.

### Example 245

4-[4-(2,4-Dichloro-phenyl)-1-(3′-trifluoromethyl-biphenyl-4-ylmethyl)-1H-imidazol-2-yl]-phenol (540 mg, 1 mmol) was alkylated with methyl bromoacetate (169 mg, 1.1 mmol) following general procedure E to give {4-[4-(2,4-dichloro-phenyl)-1-(3′-trifluoromethyl-biphenyl-4-ylmethyl)-1H-imidazol-2-yl]-phenoxy}-acetic acid methyl ester (399 mg, 66%).

{4-[4-(2,4-Dichloro-phenyl)-1-(3′-trifluoromethyl-biphenyl-4-ylmethyl)-1H-imidazol-2-yl]-phenoxy}-acetic acid methyl ester (306 mg, 0.5 mmol) was hydrolyzed following general procedure F to give {4-[4-(2,4-dichloro-phenyl)-1-(3′-trifluoromethyl-biphenyl-4-ylmethyl)-1H-imidazol-2-yl]-phenoxy}-acetic acid (255 mg, 85%).

LCMS: m/z 598 (M+H)⁺. ¹H NMR (DMSO-d₆, 400 MHz): δ 4.27 (s, 2H), 5.45 (s, 2H), 6.88 (d, 2H), 7.18 (d, 2H), 7.46 (d, 2H), 7.51 (d, 1H), 7.63-7.70 (m, 2H), 7.72 (d, 2H), 7.86 (d, 2H), 8.06 (s, 1H), 8.23 (d, 2H) ppm.

### Example 246

4-Bromo benzoic acid (10 g, 50 mol) was treated according to general procedure A using 2,4-dichlorophenacyl bromide to give 2-(4-bromo-phenyl)-4-(2,4-dichloro-phenyl)-1H-imidazole (11.2 g, 61%).

2-(4-Bromo-phenyl)-4-(2,4-dichloro-phenyl)-1H-imidazole (3.7 g, 10 mmol) was treated as described in general procedure E using methyl 4-bromomethyl benzoate (2.5 g, 11 mmol) to give 4-[2-(4-bromo-phenyl)-4-(2,4-dichloro-phenyl)-imidazol-1-ylmethyl]-benzoic acid methyl ester (3.2 g, 62%).

4-[2-(4-Bromo-phenyl)-4-(2,4-dichloro-phenyl)-imidazol-1-ylmethyl]-benzoic acid methyl ester (2.5 g, 5 mmol) was treated as described in general procedure B using 4-hydroxyphenylboronic acid (800 mg, 5.8 mmol) to give 4-[4-(2,4-dichloro-phenyl)-2-(4′-hydroxy-biphenyl-4-yl)-imidazol-1-ylmethyl]-benzoic acid methyl ester (1.9 g, 74%).

The resulted 4-[4-(2,4-Dichloro-phenyl)-2-(4′-hydroxy-biphenyl-4-yl)-imidazol-1-ylmethyl]-benzoic acid methyl ester (265 mg, 0.5 mmol) was treated with bromo ethane (60 mg, 0.52 mmol) according to general procedure E to 4-{4-(2,4-dichloro-phenyl)-2-[4′-ethoxy-biphenyl-4-yl]-imidazol-1-ylmethyl}-benzoic acid methyl ester (216 mg, 77%).

4-{4-(2,4-Dichloro-phenyl)-2-[4′-ethoxy)-biphenyl-4-yl]-imidazol-1-ylmethyl}-benzoic acid methyl ester (185 mg, 0.3 mmol) was hydrolyzed following general procedure F to give 4-{4-(2,4-dichloro-phenyl)-2-[4′-ethoxy)-biphenyl-4-yl]-imidazol-1-ylmethyl}-benzoic acid (155 mg, 86%). LCMS: m/z 544 (M+H)⁺.

### Example 247

4-{4-(2,4-dichloro-phenyl)-2-[4′-(4,4,4-trifluoro-butoxy)-biphenyl-4-yl]-imidazol-1-ylmethyl}-benzoic acid was prepared by analagous methods to those used to prepare

### Example 246

LCMS: m/z 626 (M+H)⁺.

### Example 248

4-[2-(4-Bromo-phenyl)-4-(2,4-dichloro-phenyl)-imidazol-1-ylmethyl]-benzoic acid methyl ester (2.5 g, 5 mmol) was treated as described in general procedure B using 3-amino phenylboronic acid (797 mg, 5.8 mmol) to give 4-[2-(3′-Amino-biphenyl-4-yl)-4-(2,4-dichloro-phenyl)-imidazol-1-ylmethyl]-benzoic acid methyl ester (1.9 g, 74%).

The resulted 4-[2-(3′-Amino-biphenyl-4-yl)-4-(2,4-dichloro-phenyl)-imidazol-1-ylmethyl]-benzoic acid methyl ester (264 mg, 0.5 mmol) was alkylated with 4-methylsulfonyl benzy bromide (0.99 g, 3.9 mmol) following general procedure E to give 4-{4-(2,4-Dichloro-phenyl)-2-[3′-(4-methanesulfonyl-benzylamino)-biphenyl-4-yl]-imidazol-1-ylmethyl}-benzoic acid methyl ester (1.4 g, 68%).

4-{4-(2,4-Dichloro-phenyl)-2-[4-(4-methanesulfonyl-benzyloxy)-phenyl]-imidazol-1-ylmethyl}-benzoic acid methyl ester (310 mg, 0.5 mmol) was hydrolyzed following general procedure F to give 4-{4-(2,4-Dichloro-phenyl)-2-[4-(4-methanesulfonyl-benzyloxy)-pheny]-imidazol-1-ylmethyl}-benzoic acid (325 mg, 81%).

LCMS: m/z 683 (M+H)⁺; ¹H NMR (DMSO-d₆, 400 MHz): δ 3.18 (s, 3H), 4.46 (s, 2H), 5.56 (s, 2H), 6.56 (d, 2H), 6.84 (d, 2H), 7.13 (d, 2H), 7.18 (d, 1H), 7.49 (d, 2H), 7.50 (d, 1H), 7.51-7.67 (m, 4H), 7.86-7.91 (m, 4H), 8.13 (d, 2H), 8.25 (d, 1H) ppm.

### Example 249

4-[2-(4-Bromo-phenyl)-4-(2,4-dichloro-phenyl)-imidazol-1-ylmethyl]-benzoic acid methyl ester (2.5 g, 5 mmol) was treated as described in general procedure B using 3-methanesulfonyl phenylboronic acid (1.1 g, 5.5 mmol) to give 4-[2-(3′-methanesulfonyl-biphenyl-4-yl)-4-(2,4-dichloro-phenyl)-imidazol-1-ylmethyl]-benzoic acid methyl ester (2.1 g, 71%).

4-[2-(3′-methanesulfonyl-biphenyl-4-yl)-4-(2,4-dichloro-phenyl)-imidazol-1-ylmethyl]-benzoic acid methyl ester (295 mg, 0.5 mmol) was hydrolyzed following general procedure F to give 4-[2-(3′-methanesulfonyl-biphenyl-4-yl)-4-(2,4-dichloro-phenyl)-imidazol-1-ylmethyl]-benzoic acid (227 mg, 79%). LCMS: m/z 578 (M+H)⁺.

### Example 250

4-[2-(4-Bromo-phenyl)-4-(2,4-dichloro-phenyl)-imidazol-1-ylmethyl]-benzoic acid methyl ester (2.5 g, 5 mmol) was treated as described in general procedure B using 3-trifluoromethyl phenylboronic acid (1.0 g, 5.7 mmol) to give 4-[2-(3′-trifluoro methyl-biphenyl-4-yl)-4-(2,4-dichloro-phenyl)-imidazol-1-ylmethyl]-benzoic acid methyl ester (2.0 g, 71%).

4-[2-(3′-trifluoro-methyl-biphenyl-4-yl)-4-(2,4-dichloro-phenyl)-imidazol-1-ylmethyl]-benzoic acid methyl ester (290 mg, 0.5 mmol) was hydrolyzed following general procedure F to give 4-[2-(3′-trifluoro methyl-biphenyl-4-yl)-4-(2,4-dichloro-phenyl)-imidazol-1-ylmethyl]-benzoic acid (225 mg, 79%).

LCMS: m/z 568 (M+H)⁺; ¹H NMR (DMSO-d₆, 400 MHz): δ 5.64 (d, 2H), 7.25 (d, 2H), 7.58 (d, 1H), 7.73-7.82 (m, 4H), 7.90-7.95 (m, 4H), 8.08 (d, 2H), 8.16 (d, 2H), 8.30 (s, 1H) ppm.

### Example 251

4-[2-(4-Bromo-phenyl)-4-(2,4-dichloro-phenyl)-imidazol-1-ylmethyl]-benzoic acid methyl ester (2.5 g, 5 mmol) was treated as described in general procedure B using N-boc-amino-3-methoxyphenylboronic acid (1.5 g, 5.7 mmol) to give 4-[2-(4′-tert-butoxycarbonylamino-3′-methoxy-biphenyl-4-yl)-4-(2,4-dichloro-phenyl)-imidazol-1-ylmethyl]-benzoic acid methyl ester (2.3 g, 72%).

4-[2-(4′-tert-Butoxycarbonylamino-3′-methoxy-biphenyl-4-yl)-4-(2,4-dichloro-phenyl)-imidazol-1-ylmethyl]-benzoic acid methyl ester (329 mg, 0.5 mmol) was hydrolyzed following general procedure F to give 4-[2-(4′-tert-butoxycarbonylamino-3′-methoxy-biphenyl-4-yl)-4-(2,4-dichloro-phenyl)-imidazol-1-ylmethyl]-benzoic acid (225 mg, 70%).

LCMS: m/z 645 (M+H)⁺; ¹H NMR (DMSO-d₆, 400 MHz): δ 1.44 (s, 9H), 3.86 (s, 3H), 5.36 (s, 2H), 7.09 (d, 1H), 7.11 (d, 2H), 7.21 (d, 2H), 7.47 (d, 2H), 7.51 (d, 2H), 7.57 (s, 1H), 7.64 (d, 2H), 7.74 (d, 2H), 7.81 (s, 1H), 8.16 (d, 1H) ppm.

### Example 252

4-[2-(4′-tert-Butoxycarbonylamino-3′-methoxy-biphenyl-4-yl)-4-(2,4-dichloro-phenyl)-imidazol-1-ylmethyl]-benzoic acid (64 mg, 0.1 mmol) was treated with 4N HCl following general procedure O to give 4-[2-(4′-Amino-3′-methoxy-biphenyl-4-yl)-4-(2,4-dichloro-phenyl)-imidazol-1-ylmethyl]-benzoic acid (42 mg, 77%).

LCMS: m/z 545 (M+H)⁺;

### Example 253

4-[2-(4′-Amino-3′-methoxy-biphenyl-4-yl)-4-(2,4-dichloro-phenyl)-imidazol-1-ylmethyl]-benzoic acid (109 mg, 0.2 mmol) was treated with methane sulfonyl chloride according to general procedure L to give 4-[4-(2,4-dichloro-phenyl)-2-(4′-methane-sulfonylamino-3′-methoxy-biphenyl-4-yl)-imidazol-1-ylmethyl]-benzoic acid methyl ester (79 mg, 71%).

4-[4-(2,4-Dichloro-phenyl)-2-(4′-methanesulfonylamino-3′-methoxy-biphenyl-4-yl)-imidazol-1-ylmethyl]-benzoic acid methyl ester (32 mg, 0.5 mmol) was hydrolyzed following general procedure F to give 4-[4-(2,4-Dichloro-phenyl)-2-(4′-methane-sulfonylamino-3′-methoxy-biphenyl-4-yl)-imidazol-1-ylmethyl]-benzoic acid (24 mg, 75%).

LCMS: m/z 623 (M+H)⁺; ¹H NMR (DMSO-d₆, 400 MHz): δ 2.99 (s, 3H), 3.85 (s, 3H), 5.58 (s, 2H), 7.18 (d, 2H), 7.20 (d, 2H), 7.28-7.7.36 (m, 2H), 7.50 (d, 1H), 7.67-7.78 (m, 2H), 7.80 (s, 1H), 7.90 (d, 2H), 8.16 (s, 1H), 8.29 (d, 1H), 9.01 (s, 1H) ppm.

### Example 254

4-Bromo phenyl acetic acid (11 g, 50 mol) was treated according to general procedure A using 2,4-dichlorophenacyl bromide to give 2-(4-Bromo-benzyl)-4-(2,4-dichloro-phenyl)-1H-imidazole (11.2 g, 57%).

2-(4-Bromo-benzyl)-4-(2,4-dichloro-phenyl)-1H-imidazole (3.8 g, 10 mmol) was reacted with ethyl 4-fluoro benzoate (2.5 g, 15 mmol) following general procedure I to give 4-[2-(4-Bromo-benzyl)-4-(2,4-dichloro-phenyl)-imidazol-1-yl]-benzoic acid ethyl ester (3.89 g, 74%).

4-[2-(4-Bromo-benzyl)-4-(2,4-dichloro-phenyl)-imidazol-1-yl]-benzoic acid ethyl ester (2.7 g, 5 mmol) was treated as described in general procedure B using 4-hydroxy phenylboronic acid (800 mg, 5.8 mmol) to give 4-[4-(2,4-Dichloro-phenyl)-2-(4′-hydroxy-biphenyl-4-ylmethyl)-imidazol-1-yl]-benzoic acid ethyl ester (1.9 g, 69%). 4-[4-(2,4-Dichloro-phenyl)-2-(4′-hydroxy-biphenyl-4-ylmethyl)-imidazol-1-yl]-benzoic acid ethyl ester (54 mg, 0.1 mmol) was hydrolyzed following general procedure F to give 4-[4-(2,4-Dichloro-phenyl)-2-(4′-hydroxy-biphenyl-4-ylmethyl)-imidazol-1-yl]-benzoic acid (41 mg, 80%).

LCMS: m/z 516 (M+H)⁺; ¹H NMR (DMSO-d₆, 400 MHz): δ 4.08 (s, 2H), 6.80 (d, 2H), 7.05 (d, 2H), 7.35-7.43 (m, 2H), 7.46 (d, 1H), 7.49 (d, 2H), 7.64 (d, 2H), 7.93 (s, 1H), 8.01 (d, 2H), 8.21 (d, 2H) ppm.

### Example 255

4-[2-(4-Bromo-benzyl)-4-(2,4-dichloro-phenyl)-imidazol-1-yl]-benzoic acid ethyl ester (2.7 g, 5 mmol) was treated as described in general procedure B using 3-methylsulfonyl phenylboronic acid (1.1 g, 5.5 mmol) to give 4-[4-(2,4-Dichloro-phenyl)-2-(3′-methanesulfonyl-biphenyl-4-ylmethyl)-imidazol-1-yl]-benzoic acid ethyl ester (2.1 g, 69%).

4-[4-(2,4-Dichloro-phenyl)-2-(3′-methanesulfonyl-biphenyl-4-ylmethyl)-imidazol-1-yl]-benzoic acid ethyl ester (60 mg, 0.1 mmol) was hydrolyzed following general procedure F to give 4-[4-(2,4-Dichloro-phenyl)-2-(3′-methanesulfonyl-biphenyl-4-ylmethyl)-imidazol-1-yl]-benzoic acid (41 mg, 72%).

LCMS: m/z 578 (M+H)⁺; ¹H NMR (DMSO-d₆, 400 MHz): δ 3.09 (s, 3H), 4.12 (s, 2H), 6.91 (d, 2H), 7.07 (d, 2H), 7.25-7.38 (m, 2H), 7.42 (d, 1H), 7.51 (d, 2H), 7.64 (d, 2H), 7.91 (s, 1H), 8.11 (d, 2H), 8.21 (d, 2H) ppm.

### Example 256

4-[2-(4-Bromo-benzyl)-4-(2,4-dichloro-phenyl)-imidazol-1-yl]-benzoic acid ethyl ester (2.7 g, 5 mmol) was treated as described in general procedure B using 3-trifluor methylphenylboronic acid (1.0 g, 5.3 mmol) to give 4-[4-(2,4-Dichloro-phenyl)-2-(3′-trifluoromethyl-biphenyl-4-ylmethyl)-imidazol-1-yl]-benzoic acid ethyl ester (2.1 g, 69%).

4-[4-(2,4-Dichloro-phenyl)-2-(3′-trifluoromethyl-biphenyl-4-ylmethyl)-imidazol-1-yl]-benzoic acid ethyl ester (60 mg, 0.1 mmol) was hydrolyzed following general procedure F to give 4-[4-(2,4-Dichloro-phenyl)-2-(3′-trifluoromethyl-biphenyl-4-ylmethyl)-imidazol-1-yl]-benzoic acid (49 mg, 85%).

LCMS: m/z 568 (M+H)⁺; ¹H NMR (DMSO-d₆, 400 MHz): δ 4.19 (s, 2H), 7.17 (d, 2H), 7.47 (d, 1H), 7.49-7.64 (m, 4H), 7.68 (d, 2H), 7.90 (d, 2H), 8.02 (s, 1H), 8.07 (d, 2H), 8.20 (d, 2H) ppm.

### Example 257

4-[4-(2,4-Dichloro-phenyl)-2-(4′-hydroxy-biphenyl-4-ylmethyl)-imidazol-1-yl]-benzoic acid ethyl ester (272 mg, 0.5 mmol) was alkylated with boromoethane according to general procedure E to give 4-[4-(2,4-dichloro-phenyl)-2-(4′-ethoxy-biphenyl-4-ylmethyl)-imidazol-1-yl]-benzoic acid ethyl ester (212 mg, 74%).

4-[4-(2,4-Dichloro-phenyl)-2-(4′-ethoxy-biphenyl-4-ylmethyl)-imidazol-1-yl]-benzoic acid ethyl ester (57 mg, 0.1 mmol) was hydrolyzed following general procedure F to give 4-[4-(2,4-Dichloro-phenyl)-2-(4′-ethoxy-biphenyl-4-ylmethyl)-imidazol-1-yl]-benzoic acid (49 mg, 83%). LCMS: m/z 544 (M+H)⁺;

### Example 258

4-[2-(4-Bromo-benzyl)-4-(2,4-dichloro-phenyl)-imidazol-1-yl]-benzoic acid ethyl ester (2.7 g, 5 mmol) was treated as described in general procedure B using 4-amino phenylboronic acid (0.78 g, 5.6 mmol) to give 4-[2-(4′-amino-biphenyl-4-ylmethyl)-4-(2,4-dichloro-phenyl)-imidazol-1-yl]-benzoic acid ethyl ester (1.8 g, 65%).

4-[2-(4′-Amino-biphenyl-4-ylmethyl)-4-(2,4-dichloro-phenyl)-imidazol-1-yl]-benzoic acid ethyl ester (272 mg, 0.5 mmol) was treated with isopropylsulfonyl chloride (82 mg, 0.57 mmol) following general procedure L to give 4-{4-(2,4-dichloro-phenyl)-2-[4′-(propane-2-sulfonylamino)-biphenyl-4-ylmethyl]-imidazol-1-yl}-benzoic acid ethyl ester (218 mg, 67%).

4-{4-(2,4-Dichloro-phenyl)-2-[4′-(propane-2-sulfonylamino)-biphenyl-4-ylmethyl]-imidazol-1-yl}-benzoic acid ethyl ester (65 mg, 0.1 mmol) was hydrolyzed following general procedure F to give 4-{4-(2,4-Dichloro-phenyl)-2-[4′-(propane-2-sulfonylamino)-biphenyl-4-ylmethyl]-imidazol-1-yl}-benzoic acid (49 mg, 79%).

LCMS: m/z 621 (M+H)⁺; ¹H NMR (DMSO-d₆, 400 MHz): δ 1.24 (d, 6H), 3.25 (m, 1H), 4.14 (s, 2H), 7.10 (d, 2H), 7.26 (d, 2H), 7.47-7.52 (m, 4H), 7.55 (d, 2H), 7.64 (s, 1H), 7.98 (s, 1H), 8.03 (d, 2H), 8.18 (d, 2H), 9.89 (s, 1H) ppm.

### Example 259

4-[4-(2,4-Dichloro-phenyl)-2-(4′-hydroxy-biphenyl-4-ylmethyl)-imidazol-1-yl]-benzoic acid ethyl ester (272 mg, 0.5 mmol) was alkylated with 1-bromo 4,4,4-trifluoro butane (110 mg, 0.52 mmol) according to general procedure E to give 4-{4-(2,4-Dichloro-phenyl)-2-[4′-(4,4,4-trifluoro-butoxy)-biphenyl-4-ylmethyl]-imidazol-1-yl}-benzoic acid ethyl ester (226 mg, 67%).

4-{4-(2,4-Dichloro-phenyl)-2-[4′-(4,4,4-trifluoro-butoxy)-biphenyl-4-ylmethyl]-imidazol-1-yl}-benzoic acid ethyl ester (65 mg, 0.1 mmol) was hydrolyzed following general procedure F to give 4-{4-(2,4-dichloro-phenyl)-2-[4′-(4,4,4-trifluoro-butoxy)-biphenyl-4-ylmethy]-imidazol-1-yl}-benzoic acid (50 mg, 80%).

LCMS: m/z 626 (M+H)⁺; ¹H NMR (DMSO-d₆, 400 MHz): δ 1.90 (m, 2H), 2.36 (m 2H), 4.04 (m, 2H), 4.16 (s, 2H), 6.98 (d, 2H), 7.04 (d, 2H), 7.46-7.55 (m, 4H), 7.58 (d, 2H), 7.65 (d, 2H), 8.01 (s, 1H), 8.06 (d, 2H), 8.21 (d, 2H) ppm.

### Example 260

4-[4-(2,4-Dichloro-phenyl)-2-(4′-hydroxy-biphenyl-4-ylmethyl)-imidazol-1-yl]-benzoic acid ethyl ester (272 mg, 0.5 mmol) was treated with 3-methanesulfonyl phenyl boronic acid (110 mg, 0.55 mmol) following general procedure W to give 4-{4-(2,4-dichloro-phenyl)-2-[4′-(4-methanesulfonyl-phenoxy)-biphenyl-4-ylmethyl]-imidazol-1-yl}-benzoic acid ethyl ester (247 mg, 69%).

4-{4-(2,4-Dichloro-phenyl)-2-[4′-(4-methanesulfonyl-phenoxy)-biphenyl-4-ylmethyl]-imidazol-1-yl}-benzoic acid ethyl ester (70 mg, 0.1 mmol) was hydrolyzed following general procedure F to give 4-{4-(2,4-Dichloro-phenyl)-2-[4′-(4-methane-sulfonyl-phenoxy)-biphenyl-4-ylmethy]-imidazol-1-yl}-benzoic acid (61 mg, 84%).

LCMS: m/z 670 (M+H)⁺; ¹H NMR (DMSO-d₆, 400 MHz): δ 4.19 (s, 2H), 7.13 (d, 2H), 7.16 (d, 2H), 7.21 (d, 2H), 7.49-7.57 (m, 6H), 7.59 (s, 1H), 7.66 (d, 2H), 7.69 (d, 2H), 7.90 (d, 2H), 8.06 (d, 2H), 8.19 (d, 2H) ppm.

### Example 261

4-[4-(2,4-Dichloro-phenyl)-2-(4′-hydroxy-biphenyl-4-ylmethyl)-imidazol-1-yl]-benzoic acid ethyl ester (272 mg, 0.5 mmol) was treated with 4-tert-butyl phenyl boronic acid (98 mg, 0.55 mmol) following general procedure W to give 4-[2-[4′-(4-tert-Butyl-phenoxy)-biphenyl-4-ylmethyl]-4-(2,4-dichloro-phenyl)-imidazol-1-yl]-benzoic acid ethyl ester (227 mg, 66%).

4-2-[4′-(4-tert-Butyl-phenoxy)-biphenyl-4-ylmethyl]-4-(2,4-dichloro-phenyl)-imidazol-1-yl]-benzoic acid ethyl ester (68 mg, 0.1 mmol) was hydrolyzed following general procedure F to give 4-2-[4′-(4-tert-Butyl-phenoxy)-biphenyl-4-ylmethyl]-4-(2,4-dichloro-phenyl)-imidazol-1-yl]-benzoic acid (59 mg, 82%).

LCMS: m/z 670 (M+H)⁺; ¹H NMR (DMSO-d₆, 400 MHz): δ 1.28 (s, 9H), 4.16 (s, 2H), 6.96 (dd, 2H), 7.11 (d, 2H), 7.40 (d, 2H), 7.42 (d, 2H), 7.48 (d, 2H), 7.50 (d, 2H), 7.51-7.66 (m, 4H), 8.01 (dd, 2H), 8.21 (d, 2H) ppm.

### Example 262

4-[2-(4′-Amino-biphenyl-4-ylmethyl)-4-(2,4-dichloro-phenyl)-imidazol-1-yl]-benzoic acid ethyl ester (272 mg, 0.5 mmol) was treated with 3-trifluoromethyl benzene sulfonyl chloride (136 mg, 0.57 mmol) following general procedure L to give 4-{4-(2,4-Dichloro-phenyl)-2-[4′-(3-trifluoromethyl-benzenesulfonylamino)-biphenyl-4-ylmethyl]-imidazol-1-yl}-benzoic acid methyl ester (248 mg, 66%).

4-{4-(2,4-Dichloro-phenyl)-2-[4′-(3-trifluoromethyl-benzenesulfonylamino)-biphenyl-4-ylmethyl]-imidazol-1-yl}-benzoic acid methyl ester (75 mg, 0.1 mmol) was hydrolyzed following general procedure F to give 4-{4-(2,4-Dichloro-phenyl)-2-[4′-(3-trifluoromethyl-benzenesulfonylamino)-biphenyl-4-ylmethyl]-imidazol-1-yl}-benzoic acid (59 mg, 76%).

LCMS: m/z 621 (M+H)⁺; ¹H NMR (DMSO-d₆, 400 MHz): δ 4.14 (s, 2H), 7.08 (d, 2H), 7.12 (d, 2H), 7.43 (d, 2H), 7.45-7.50 (m, 4H), 7.51 (d, 2H), 7.65 (d, 2H), 7.80 (s, 1H), 8.01 (d, 2H), 8.04 (d, 2H), 8.17 (d, 2H) ppm.

### Example 263

4-[4-(2,4-Dichloro-phenyl)-2-(4′-hydroxy-biphenyl-4-ylmethyl)-imidazol-1-yl]-benzoic acid ethyl ester (272 mg, 0.5 mmol) was treated with 4-trifluoromethyl phenyl boronic acid (110 mg, 0.57 mmol) following general procedure W to give 4-{4-(2,4-Dichloro-phenyl)-2-[4′-(4-trifluoromethyl-phenoxy)-biphenyl-4-yl-methyl]imidazol-yl}-benzoic acid ethyl ester (217 mg, 65%).

4-{4-(2,4-Dichloro-phenyl)-2-[4′-(4-trifluoromethyl-phenoxy)-biphenyl-4-yl-methyl]imidazol-yl}-benzoic acid ethyl ester (68 mg, 0.1 mmol) was hydrolyzed following general procedure F to give 4-{4-(2,4-Dichloro-phenyl)-2-[4′-(4-trifluoromethyl-phenoxy)-biphenyl-4-yl-methyl]imidazol-yl}-benzoic acid (57 mg, 81%).

LCMS: m/z 660 (M+H)⁺; ¹H NMR (DMSO-d₆, 400 MHz): δ 4.19 (s, 2H), 7.13-7.19 (m, 4H), 7.48-7.59 (m 4H), 7.61-7.75 (m, 6H), 8.05 (d, 2H), 8.09 (d, 2H), 8.21 (d, 2H) ppm.

By analagous methods to those used to prepare Example 265, the following compounds were synthesized:

| Example | Name | LC/MS (m/z) |
|---|---|---|
| 264 | 4-{4-(2,4-Dichloro-phenyl)-2-[4′-(4- | 660 (M + H)⁺ |
| | trifluoromethyl-phenoxy)-biphenyl-4-yl- | |
| | methyl]imidazol-1-yl}-benzoic acid | |
| 265 | 4-{4-(2,4-Dichloro-phenyl)-2-[4′-(4- | 684 (M + H)⁺ |
| | methanesulfonyl-benzyloxy)-biphenyl-4- | |
| | ylmethyl]-imidazol-1-yl}-benzoic acid | |
| 266 | 4-{4-(2,4-Dichloro-phenyl)-2-[3′-(4- | 660 (M + H)⁺ |
| | trifluoromethyl-phenoxy)-biphenyl-4-yl- | |
| | methyl]imidazo1-yl}-benzoic acid | |

### Example 267

N-{4-[2-[4′-(4-tert-Butyl-phenoxy)-biphenyl-4-ylmethyl]-4-(2,4-dichloro-phenyl)-imidazol-1-yl]-benzoyl}-methanesulfonamide (17 mg, 47%) was prepared from methanesulfonamide (5 mg, 0.045 mmol) and 4-[2-[4′-(4-tert-Butyl-phenoxy)-biphenyl-4-ylmethyl]-4-(2,4-dichloro-phenyl)-imidazol-1-yl]-benzoic acid (33 mg, 0.05 mmol) according to the general procedure AA.

LCMS: m/z 725 (M+H)⁺; ¹H NMR (DMSO-d₆, 400 MHz): δ 1.27 (s, 9H), 3.47 (s, 3H), 4.16 (s, 2H), 6.96 (dd, 2H), 7.02 (d, 2H), 7.14 (d, 2H), 7.26 (d, 2H), 7.39-7.47 (m, 4H), 7.51-7.63 (m, 4H), 7.81 (d, 2H), 7.87 (s, 1H), 8.19 (d, 1H) ppm.

By analagous methods to those used to prepare Example 267, the following compounds were synthesized:

| Example | Name | LC/MS (m/z) |
|---|---|---|
| 268 | N-(4-{4-(2,4-Dichloro-phenyl)-2-[4′-(3- | 815 (M + H)⁺ |
| | trifluoromethyl-phenoxy)-biphenyl-4- | |
| | ylmethyl]-imidazol-1-yl}-benzoyl)-N-N- | |
| | dimethanesulfonamide | |
| 269 | N-(4-{4-(2,4-Dichloro-phenyl)-2-[4′-(3- | 737 (M + H)⁺ |
| | trifluoromethyl-phenoxy)-biphenyl-4- | |
| | ylmethyl]-imidazol-1-yl}-benzoyl)- | |
| | methanesulfonamide | |
| 270 | Ethanesulfonic acid 4-[2-[4′-(4-tert-butyl- | 739 (M + H)⁺ |
| | phenoxy)-biphenyl-4-ylmethyl]-4-(2,4- | |
| | dichloro-phenyl)-imidazol-1-yl]- | |
| | benzoylamide | |

### Example 271

4-{4-(2,4-Dichloro-phenyl)-2-[4′-(3-trifluoromethyl-phenoxy)-biphenyl-4-ylmethyl]-imidazol-1-yl}-N-methyl-benzamide (19 mg, 59%) was prepared from 2.0 M methyl amine in methanol solution and 4-{4-(2,4-dichloro-phenyl)-2-[4′-(3-trifluoromethyl-phenoxy)-biphenyl-4-ylmethyl]-imidazol-1-yl}-benzoic acid (33 mg, 0.05 mmol) according to the general procedure AA. LCMS: m/z 673 (M+H)⁺;

### Example 272

2-(4-Bromo-benzyl)-4-(2,4-dichloro-phenyl)-1H-imidazole (3.8 g, 10 mmol) was reacted with 5-fluoro-2-trifluoromethyl-benzoic acid methyl ester (3.4 g, 15 mmol) following general procedure I to give 5-[2-(4-bromo-benzyl)-4-(2,4-dichloro-phenyl)-imidazol-1-yl]-2-trifluoromethyl-benzoic acid methyl ester (3.9 g, 67%).

5-[2-(4-Bromo-benzyl)-4-(2,4-dichloro-phenyl)-imidazol-1-yl]-2-trifluoromethyl-benzoic acid methyl ester (2.9 g, 5 mmol) was treated as described in general procedure B using 4-hydroxy phenylboronic acid (800 mg, 5.8 mmol) to give 5-[4-(2,4-Dichloro-phenyl)-2-(4′-hydroxy-biphenyl-4-ylmethyl)-imidazol-1-yl]-2-trifluoromethyl-benzoic acid methyl ester (2.1 g, 70%).

5-[4-(2,4-Dichloro-phenyl)-2-(4′-hydroxy-biphenyl-4-ylmethyl)-imidazol-1-yl]-2-trifluoromethyl-benzoic acid methyl ester (60 mg, 0.1 mmol) was hydrolyzed following general procedure F to give 5-[4-(2,4-Dichloro-phenyl)-2-(4′-hydroxy-biphenyl-4-ylmethyl)-imidazol-1-yl]-2-trifluoromethyl-benzoic acid (44 mg, 76%).

LCMS: m/z 584 (M+H)⁺; ¹H NMR (DMSO-d₆, 400 MHz): δ 4.16 (s, 2H), 6.55 (s, 1H), 6.77 (s, 2H), 7.11 (d, 2H), 7.37 (d, 2H), 7.49 (d, 2H), 7.51 (d, 2H), 7.66 (s, 1H), 7.86 (s, 1H), 7.96 (s, 1H), 8.11 (d, 2H), 9.52 (s, 1H) ppm.

### Example 273

5-[4-(2,4-Dichloro-phenyl)-2-(4′-hydroxy-biphenyl-4-ylmethyl)-imidazol-1-yl]-2-trifluoromethyl-benzoic acid methyl ester (600 mg, 1 mmol) was treated with 4-fluoronitro benzene (160 mg, 1.1 mmol) according to general procedure I to give 5-[2-[4′-(4-Nitro-phenoxy)-biphenyl-4-ylmethyl]-4-(2,4-dichloro-phenyl)-imidazol-1-yl]-2-trifluoromethyl-benzoic acid methyl ester (521 mg, 72%).

5-[2-[4′-(4-Nitro-phenoxy)-biphenyl-4-ylmethyl]-4-(2,4-dichloro-phenyl)-imidazol-1-yl]-2-trifluoromethyl-benzoic acid methyl ester (72 mg, 0.1 mmol) was hydrolyzed following general procedure F to give 5-[2-[4′-(4-Nitro-phenoxy)-biphenyl-4-ylmethyl]-4-(2,4-dichloro-phenyl)-imidazol-1-yl]-2-trifluoromethyl-benzoic acid (57 mg, 81% yield).

LCMS: m/z 705 (M+H)⁺;

### Example 274

5-[2-[4′-(4-Nitro-phenoxy)-biphenyl-4-ylmethyl]-4-(2,4-dichloro-phenyl)-imidazol-1-yl]-2-trifluoromethyl-benzoic acid methyl ester (720 mg, 1 mmol) was reduced according to general procedure K to give 5-[2-[4′-(4-amino-phenoxy)-biphenyl-4-ylmethyl]-4-(2,4-dichloro-phenyl)-imidazol-1-yl]-2-trifluoromethyl-benzoic acid methyl ester (586 mg, 85%).

5-[2-[4′-(4-amino-phenoxy)-biphenyl-4-ylmethyl]-4-(2,4-dichloro-phenyl)-imidazol-1-yl]-2-trifluoromethyl-benzoic acid methyl ester (345 mg, 0.5 mmol) was treated with isobutylsulfonyl chloride (89 mg, 0.56 mmol) following general procedure L to give 5-(4-(2,4-dichloro-phenyl)-2-{4′-[4-(2-methyl-propane-1-sulfonylamino)-phenoxy]-biphenyl-4-ylmethyl}-imidazol-1-yl)-2-trifluoromethyl-benzoic acid methyl ester (291 mg, 71%).

5-(4-(2,4-Dichloro-phenyl)-2-{4′-[4-(2-methyl-propane-1-sulfonylamino)-phenoxy]-biphenyl-4-ylmethyl}-imidazol-1-yl)-2-trifluoromethyl-benzoic acid methyl ester (81 mg, 0.1 mmol) was hydrolyzed following general procedure F to give 5-(4-(2,4-dichloro-phenyl)-2-{4′-[4-(2-methyl-propane-1-sulfonylamino)-phenoxy]-biphenyl-4-ylmethyl}-imidazol-1-yl)-2-trifluoromethyl-benzoic acid (62 mg, 78%).

LCMS: m/z 795 (M+H)⁺; ¹H NMR (DMSO-d₆, 400 MHz): δ 1.11 (d, 6H), 2.12 (m, 1H), 4.14 (s, 2H), 4.19 (m, 2H), 7.02 (d, 2H), 7.06 (d, 2H), 7.17 (d, 2H), 7.26 (d, 2H), 7.47-7.52 (m, 2H), 7.60 (d, 2H), 7.62 (d, 2H), 7.75 (d, 2H), 8.01 (s, 1H), 8.16 (d, 2H) ppm.

By analagous methods to those used to prepare Example 274, the following compounds were synthesized:

| Example | Name | LC/MS (m/z) |
|---|---|---|
| 275 | 5-{4-(2,4-dichloro-phenyl)-2-[4′-(4- | 795 (M + H)⁺ |
| | ethanesulfonylamino-phenoxy)-biphenyl-4- | |
| | ylmethyl]-imidazol-1-yl}-2- | |
| | trifluoromethyl-benzoic acid | |
| 276 | 5-(4-(2,4-dichloro-phenyl)-2-{4′-[4- | 809 (M + H)⁺ |
| | (pentane-1-sulfonylamino)-phenoxy]- | |
| | biphenyl-4-ylmethyl}-imidazol-1-yl)- | |
| | 2-trifluoromethyl-benzoic acid | |

### Example 277

5-[4-(2,4-Dichloro-phenyl)-2-(4′-hydroxy-biphenyl-4-ylmethyl)-imidazol-1-yl]-2-trifluoromethyl-benzoic acid methyl ester (299 mg, 0.5 mmol) was treated with 4-tert-butylphenyl boronic acid (99 mg, 0.55 mmol) following general procedure W to give 5-[2-[4′-(4-tert-Butyl-phenoxy)-biphenyl-4-ylmethyl]-4-(2,4-dichloro-phenyl)-imidazol-1-yl]-2-trifluoromethyl-benzoic acid methyl ester (231 mg, 64%).

5-[2-[4′-(4-tert-Butyl-phenoxy)-biphenyl-4-ylmethyl]-4-(2,4-dichloro-phenyl)-imidazol-1-yl]-2-trifluoromethyl-benzoic acid methyl ester (73 mg, 0.1 mmol) was hydrolyzed following general procedure F to give 5-[2-[4′-(4-tert-butyl-phenoxy)-biphenyl-4-ylmethyl]-4-(2,4-dichloro-phenyl)-imidazol-1-yl]-2-trifluoromethyl-benzoic acid (59 mg, 83%).

LCMS: m/z 716 (M+H)⁺; ¹H NMR (DMSO-d₆, 400 MHz): δ 1.29 (s, 9H), 4.18 (s, 2H), 6.98 (d, 2H), 7.10 (d, 2H), 7.41 (d, 2H), 7.47 (d, 2H), 7.51 (d, 2H), 7.53 (d, 2H), 7.57-7.67 (m, 4H), 8.01 (dd, 2H), 8.19 (s, 1H) ppm.

### Example 278

2-(4-Bromo-benzyl)-4-(2,4-dichloro-phenyl)-1H-imidazole (3.8 g, 10 mmol) was reacted with 4-fluoro-2-nitro benzoic acid methyl ester (2.9 g, 15 mmol) following general procedure I to give 2-nitro-4-[2-(4-bromo-benzyl)-4-(2,4-dichloro-phenyl)-imidazol-1-yl]-benzoic acid methyl ester (3.8 g, 67%).

2-Nitro-4-[2-(4-bromo-benzyl)-4-(2,4-dichloro-phenyl)-imidazol-1-yl]-benzoic acid methyl ester (2.8 g, 5 mmol) was reduced according to general procedure K to give 2-amino-4-[2-(4-bromo-benzyl)-4-(2,4-dichloro-phenyl)-imidazol-1-yl]-benzoic acid methyl ester (1.81 g, 68%).

2-amino-4-[2-(4-bromo-benzyl)-4-(2,4-dichloro-phenyl)-imidazol-1-yl]-benzoic acid methyl ester (1.78 g, 3.3 mmol) was treated with methylsulfonyl chloride (405 mg, 3.5 mmol) following general procedure L to give 4-[2-(4-Bromo-benzyl)-4-(2,4-dichloro-phenyl)-imidazol-1-yl]-2-methanesulfonyl amino-benzoic acid methyl ester (1.39 g, 68%).

4-[2-(4-Bromo-benzyl)-4-(2,4-dichloro-phenyl)-imidazol-1-yl]methanesulfonyl amino-benzoic acid methyl ester (305 mg, 0.5 mmol) was treated as described in general procedure B using 4-hydroxy phenylboronic acid (80 mg, 0.57 mmol) to give -[4-(2,4-dichloro-phenyl)-2-(4′-hydroxy-biphenyl-4-ylmethyl)-imidazol-1-yl]-2-methane-sulfonylamino-benzoic acid methyl ester (209 mg, 67%).

4-[4-(2,4-Dichloro-phenyl)-2-(4′-hydroxy-biphenyl-4-ylmethyl)-imidazol-1-yl]-2-methanesulfonylamino-benzoic acid methyl ester (63 mg, 0.1 mmol) was hydrolyzed following general procedure F to give 4-[4-(2,4-Dichloro-phenyl)-2-(4′-hydroxy-biphenyl-4-ylmethyl)-imidazol-1-yl]-2-methanesulfonylamino-benzoic acid (47 mg, 77%).

LCMS: m/z 609 (M+H)⁺;

By analagous methods to those used to prepare Example 278, the following compounds were synthesized:

| Example | Name | LC/MS (m/z) |
|---|---|---|
| 279 | 4-[2-(4′-tert-Butyl-biphenyl-4-ylmethyl)-4- | 649 (M + H)⁺ |
| | (2,4-dichloro-phenyl)-imidazol-1-yl]-2- | |
| | methanesulfonylamino-benzoic acid | |
| 280 | 4-[4-(2,4-dichloro-phenyl)-2-(4′- | 661 (M + H)⁺ |
| | trifluoromethyl-biphenyl-4-ylmethyl)- | |
| | imidazol-1-yl]-2-methanesulfonyl- | |
| | amino-benzoic acid acid | |
| 281 | 4-[2-(4′-tert-Butyl-biphenyl-4-ylmethyl)-4- | 649 (M + H)⁺ |
| | (2,4-dichloro-phenyl)-imidazol-1-yl]-2- | |
| | trifluoromethanesulfonylamino- | |
| | benzoic acid | |

### Example 282

4-[4-(2,4-Dichloro-phenyl)-2-(4′-hydroxy-biphenyl-4-ylmethyl)-imidazol-1-yl]-2-methanesulfonylamino-benzoic acid methyl ester (311 mg, 0.5 mmol) was treated with 4-tert-butyl phenyl boronic acid (99 mg, 0.55 mmol) following general procedure W to give 4-[2-[4′-(4-tert-Butyl-phenoxy)-biphenyl-4-ylmethyl]-4-(2,4-dichloro-phenyl)-imidazol-1-yl]-2-methanesulfonylamino-benzoic acid methyl ester (241 mg, 64%).

4-[2-[4′-(4-tert-Butyl-phenoxy)-biphenyl-4-ylmethyl]-4-(2,4-dichloro-phenyl)-imidazol-1-yl]-2-methanesulfonylamino-benzoic acid methyl ester (75 mg, 0.1 mmol) was hydrolyzed following general procedure F to give 4-[2-[4′-(4-tert-Butyl-phenoxy)-biphenyl-4-ylmethyl]-4-(2,4-dichloro-phenyl)-imidazol-1-yl]-2-methanesulfonylamino-benzoic acid (61 mg, 83%).

LCMS: m/z 741 (M+H)⁺; ¹H NMR (DMSO-d₆, 400 MHz): δ 1.29 (s, 9H), 3.34 (s, 3H), 4.18 (s, 2H), 6.97 (d, 2H), 7.10 (d, 2H), 7.41 (d, 2H), 7.46 (d, 2H), 7.51 (d, 2H), 7.53 (d, 2H), 7.55-7.67 (m, 4H), 8.01 (dd, 2H), 8.19 (s, 1H) ppm.

### Example 283

4-Bromophenoxyacetic acid (23.1 g, 10 m mol) was treated according to general procedure A using 2,4-dichlorophenacyl bromide to give 2-(4-bromo-phenoxymethyl)-4-(2,4-dichloro-phenyl)-1H-imidazole (14.3 g, 36%). LCMS: m/z 399 (M+H)⁺.

2-(4-Bromo-phenoxymethyl)-4-(2,4-dichloro-phenyl)-1H-imidazole (11.9 g, 30 mmol) was treated as described in general procedure E using bromoethane to give 2-(4-Bromo-phenoxymethyl)-4-(2,4-dichloro-phenyl)-1-ethyl-1H-imidazol (10.3 g, 82% yield).

LCMS: m/z 427 (M+H).

2-(4-Bromo-phenoxymethyl)-4-(2,4-dichloro-phenyl)-1-ethyl-1H-imidazol (430 mg, 1 mmol) was treated as described in general procedure B using 3-hydroxy benzeneboronic acid (200 mg, 1.4 mmol) to give 4′-[4-(2,4-Dichloro-phenyl)-1-ethyl-1H-imidazol-2-ylmethoxy]-biphenyl-3-ol (270 mg, 61% yield).

LCMS: m/z 439 (M+H)⁺.

4′-[4-(2,4-Dichloro-phenyl)-1-ethyl-1H-imidazol-2-ylmethoxy]-biphenyl-3-ol (25 mg, 0.05 mmol) was treated with methyl 4-bromobutyrate (13 mg, 0.07 m mole) according to the general procedure E to give 4-{4′-[4-(2,4-Dichloro-phenyl)-1-ethyl-1H-imidazol-2-ylmethoxy]-biphenyl-3-yloxy}-butyric acid methyl ester (20 mg, 61%).

4-{4′-[4-(2,4-Dichloro-phenyl)-1-ethyl-1H-imidazol-2-ylmethoxy]-biphenyl-3-yloxy}-butyric acid (12 mg, 70%) is prepared according to general procedure F using 4-{4′-[4-(2,4-Dichloro-phenyl)-1-ethyl-1H-imidazol-2-ylmethoxy]-biphenyl-3-yloxy}-butyric acid methyl ester (18 mg, 0.03 m mole).

LCMS: m/z 526 (M+H)⁺; ¹H NMR (DMSO-d₆, 400 MHz): δ 1.48 (t, 3H), 2.05 (q, 2H), 2.48 (q, 2H), 4.12-4.15 (m, 2H), 4.22-4.25 (m, 2H), 5.35 (s, 2H), 6.95 (d, 1H), 7.23 (d, 2H), 7.43 (d, 1H), 7.54 (d, 1H), 7.56-7.73 (m, 4H), 8.06 (s, 2H), 8.21 (d, 1H) ppm.

### Example 284

4′-[4-(2,4-Dichloro-phenyl)-1-ethyl-1H-imidazol-2-ylmethoxy]-biphenyl-3-ol (44 mg, 0.1 mmol) was reacted with 5-fluoro-2-trifluoromethyl-benzoic acid methyl ester (34 mg, 1.5 mmol) following general procedure I to give 5-{4′-[4-(2,4-dichloro-phenyl)-1-ethyl-1H-imidazol-2-ylmethoxy]-biphenyl-3-yloxy}-2-trifluoromethyl-benzoic acid methyl ester (41 mg, 64%).

5-{4′-[4-(2,4-Dichloro-phenyl)-1-ethyl-1H-imidazol-2-ylmethoxy]-biphenyl-3-yloxy}-2-trifluoromethyl-benzoic acid methyl ester (65 mg, 0.1 mmol) was hydrolyzed following general procedure F to give 5-{4′-[4-(2,4-dichloro-phenyl)-1-ethyl-1H-imidazol-2-ylmethoxy]-biphenyl-3-yloxy}-2-trifluoromethyl-benzoic acid (51 mg, 80%).

LCMS: m/z 628 (M+H)⁺; ¹H NMR (DMSO-d₆, 400 MHz): δ 1.36 (t, 3H), 4.10 (q, 2H), 5.26 (s, 2H), 6.99 (d, 2H), 7.05 (d, 2H), 7.16 (d, 2H), 7.38 (s, 1H), 7.44 (d, 1H), 7.46-7.50 (m, 2H), 7.58-7.66 (m, 2H), 7.96 (s, 1H), 8.13 (d, 2H) ppm.

### Example 285

2-(4-Bromo-phenoxymethyl)-4-(2,4-dichloro-phenyl)-1H-imidazol (4 g, 10 mmol) was treated with methyl 4-bromomethyl benzoate (3.5 g, 15 mmol) following general procedure E to give 4-[2-(4-Bromo-phenoxymethyl)-4-(2,4-dichloro-phenyl)-imidazol-1-ylmethyl]-benzoic acid methyl ester (4.12 g, 76%).

4-[2-(4-Bromo-phenoxymethyl)-4-(2,4-dichloro-phenyl)-imidazol-1-ylmethyl]-benzoic acid methyl ester (274 mg, 0.5 mmol) was treated as described in general procedure B using 4-trifluoromethyl benzeneboronic acid (108 mg, 0.57 mmol) to give 4-[4-(2,4-dichloro-phenyl)-2-(4′-trifluoromethyl-biphenyl-4-yloxymethyl)-imidazol 1-ylmethyl]-benzoic acid methyl ester (221 mg, 72%).

4-[4-(2,4-Dichloro-phenyl)-2-(4′-trifluoromethyl-biphenyl-4-yloxymethyl)imidazol 1-ylmethyl]-benzoic acid methyl ester (62 mg, 0.1 mmol) was hydrolyzed following general procedure F to give 4-[4-(2,4-Dichloro-phenyl)-2-(4′-trifluoromethyl-biphenyl-4 yloxymethyl)-imidazol-1-ylmethyl]-benzoic acid (50 mg, 80%).

LCMS: m/z 598 (M+H)⁺; ¹H NMR (DMSO-d₆, 400 MHz): δ 5.25 (s, 2H), 5.47 (s, 2H), 7.05 (d, 2H), 7.29 (d, 2H), 7.46 (d, 1H), 7.48 (d, 2H), 7.63-7.68 (m, 2H), 7.74 (d, 1H), 7.83 (d, 2H), 7.88 (d, 2H), 8.05 (s, 1H), 8.14 (d, 1H) ppm.

By analagous methods to those used to prepare Example 285, the following compounds were synthesized:

| Example | Name | LC/MS (m/z) |
|---|---|---|
| 286 | 4-[4-(2,4-Dichloro-phenyl)-2-(3′- | 598 (M + H)⁺ |
| | trifluoromethyl-biphenyl-4yloxymethyl)- | |
| | imidazol-1-ylmethyl]-benzoic acid | |
| 287 | 4-[4-(2,4-Dichloro-phenyl)-2-(4′- | 608 (M + H)⁺ |
| | methanesulfonylbiphenyl-4-yloxymethyl) | |
| | imidazol-1-ylmethyl]-benzoic acid | |
| | methyl ester | |
| 288 | 4-[4-(2,4-Dichloro-phenyl)-2-(3′- | 608 (M + H)⁺ |
| | methanesulfonyl biphenyl-4-yloxymethyl) | |
| | imidazol-1-ylmethyl]-benzoic acid methyl | |
| | ester | |

### Example 289

4-[2-(4-Bromo-phenoxymethyl)-4-(2,4-dichloro-phenyl)-imidazol-1-ylmethyl]-benzoic acid methyl ester (274 mg, 0.5 mmol) was treated as described in general procedure B using 4-amino phenylboronic acid (78 mg, 0.56 mmol) to give 4-[2-(4′-Amino-biphenyl-4-yloxymethyl)-4-(2,4-dichloro-phenyl)-imidazol-1-ylmethyl]-benzoic acid methyl ester (201 mg, 71%).

4-[2-(4′-Amino-biphenyl-4-yloxymethyl)-4-(2,4-dichloro-phenyl)-imidazol-1-ylmethyl]-benzoic acid methyl ester (186 mg, 0.33 mmol) was reacted with methanesulfonyl chloride (40 mg, 0.35 mmol) following general procedure L to give 4-[4-(2,4-Dichloro-phenyl)-2-(4′-methanesulfonylamino-biphenyl-4-yloxymethyl)-imidazol-1-ylmethyl]-benzoic acid methyl ester (167 mg, 79%).

4-[4-(2,4-Dichloro-phenyl)-2-(4′-methanesulfonylamino-biphenyl-4-yloxymethyl) imidazol-1ylmethyl]-benzoic acid methyl ester (64 mg, 0.1 mmol) was hydrolyzed following general procedure F to give 4-[4-(2,4-dichloro-phenyl)-2-(4′-methanesulfonyl amino-biphenyl-4-yloxymethyl)-imidazol-lylmethyl]-benzoic acid methyl ester (51 mg, 78%).

LCMS: m/z 623 (M+H)⁺; ¹H NMR (DMSO-d₆, 400 MHz): δ 3.01 (s, 3H), 5.24 (s, 2H), 5.49 (s, 2H), 6.99 (d, 2H), 7.24 (d, 2H), 7.32 (d, 2H), 7.48-7.57 (m, 2H), 7.59 (d, 2H), 7.65 (d, 2H), 7.90 (d, 2H), 8.08 (s, 1H), 8.13 (d, 1H), 9.8 (s, 1H) ppm.

By analagous methods to those used to prepare Example 289, the following compounds were synthesized:

| Example | Name | LC/MS (m/z) |
|---|---|---|
| 290 | 4-{4-(2,4-Dichloro-phenyl)-2-[4′-(2,2,2- | 691 (M + H)⁺ |
| | trifluoro-ethanesulfonylamino)-biphenyl- | |
| | 4-yloxymethyl]-imidazol-1-ylmethyl}- | |
| | benzoic acid | |
| 291 | 4-[4-(2,4-dichloro-phenyl)-2-(4′-isopropoxy- | 691 (M + H)⁺ |
| | carbonylamino-biphenyl-4-yloxymethyl)- | |
| | imidazol-1-ylmethyl]-benzoic acid | |
| 292 | 4-[2-(4′-tert-butoxycarbonylamino-3′- | 675 (M + H)⁺ |
| | methoxy-biphenyl-4-yloxymethyl)-4-(2,4- | |
| | dichloro-phenyl)-imidazol-1-ylmethyl]- | |
| | benzoic | |

### Example 293

The compound of Example 292 (69 mg, 0.1 mmol) was treated with 2N HCl in dioxane following general procedure to give 4-[2-(4′-amino-3′-methoxy-biphenyl-4-yloxymethyl)-4-(2,4-dichloro-phenyl)-imidazol-1-ylmethyl]-benzoic acid (41 mg, 70% yield).

LCMS: m/z 575 (M+H)⁺; ¹H NMR (DMSO-d₆, 400 MHz): δ 3.98 (s, 3H), 5.36 (s, 2H), 5.56 (s, 2H), 6.99 (d, 2H), 7.03 (d, 1H), 7.25 (d, 1H), 7.36 (d, 2H), 7.47 (d, 1H), 7.49 (d, 1H), 7.53 (d, 1H), 7.64 (s, 1H), 7.71 (s, 1H), 7.91 (d, 2H), 7.93 (d, 2H), 8.10 (d, 1H), 8.17 (s, 1H) ppm.

### Example 294

2-[2-(4-Bromo-phenyl)-(E)-vinyl]-4-(2,4-dichloro-phenyl)-1H-imidazole (4.0 g, 10 mmol) was reacted with ethyl 4-fluoro benzoate (2.5 g, 15 mmol) following general procedure I to give 4-[2-[2-(4-bromo-phenyl)-(E)-vinyl]-4-(2,4-dichloro-phenyl)-imidazol-1-yl]-benzoic acid ethyl ester (3.9 g, 70%).

4-[2-[2-(4-Bromo-phenyl)-(E)-vinyl]-4-(2,4-dichloro-phenyl)-imidazol-1-yl]-benzoic acid ethyl ester (272 mg, 0.5 mmol) was coupled with 3-(methanesulfonyl)-phenyl boronic acid (110 mg, 0.55 mmol) following general procedure B to give 4-{4-(2,4-dichloro-phenyl)-2-[2-(3′-methanesulfonyl-biphenyl-4-yl)-(E)-vinyl]-imidazol-1-yl}-benzoic acid ethyl ester (226 mg, 73%).

4-{4-(2,4-Dichloro-phenyl)-2-[2-(3′-methanesulfonyl-biphenyl-4-yl)-(E)-vinyl]-imidazol-1-yl}-benzoic acid ethyl ester (62 mg, 0.1 mmol) was hydrolyzed according to general procedure F to give 4-{4-(2,4-dichloro-phenyl)-2-[2-(3′-methanesulfonyl-biphenyl-4-yl)-(E)-vinyl]-imidazol-1-yl}-benzoic acid (46 mg, 78%).

LCMS: 590 (M+H)^{+ 1}H NMR (DMSO, 400 MHz): δ 3.27 (s, 3H), 6.97 (s, 1H), 7.01 (s, 1H), 7.55 (d, 2H), 7.57 (d, 2H), 7.69-7.79 (m, 4H), 7.90 (d, 2H), 8.04 (d, 2H), 8.16-8.18 (m, 2H), 8.29 (s, 1H), 8.32 (s, 1H) ppm.

### Example 295

4-{4-(2,4-Dichloro-phenyl)-2-[2-(3′-trifluoromethyl-biphenyl-4-yl)-(E)-vinyl]-imidazol-1-yl}-benzoic acid was prepared by analagous methods to those used to prepare Example 294. LCMS: 580 (M+H)⁺.

### Example 296

4-[2-[2-(4-Bromo-phenyl)-(E)-vinyl]-4-(2,4-dichloro-phenyl)-imidazol-1-yl]-benzoic acid ethyl ester (272 mg, 0.5 mmol) was coupled with 3-(amino)-phenyl boronic acid (75 mg, 0.55 mmol) following general procedure B to give 4-{4-(2,4-Dichloro-phenyl)-2-[2-(3′-amino-biphenyl-4-yl)-(E)-vinyl]-imidazol-1-yl}-benzoic acid ethyl ester (212 mg, 76%).

4-{4-(2,4-Dichloro-phenyl)-2-[2-(3′-amino-biphenyl-4-yl)-(E)-vinyl]-imidazol-1-yl}-benzoic acid ethyl ester (185 mg, 0.33 mmol) was reacted with methanesulfonyl chloride (40 mg, 0.35 mmol) following general procedure L to give 4-{4-(2,4-Dichloro-phenyl)-2-[2-(3′-methanesulfonylamino-biphenyl-4-yl)-(E)-vinyl]-imidazol-1-yl}-benzoic acid ethyl ester (159 mg, 75%).

4-{4-(2,4-Dichloro-phenyl)-2-[2-(3′-methanesulfonylamino-biphenyl-4-yl)-(E)-vinyl]-imidazol-1-yl}-benzoic acid ethyl ester (64 mg, 0.1 mmol) was hydrolyzed according to general procedure F to give 4-{4-(2,4-Dichloro-phenyl)-2-[2-(3′-methanesulfonylamino-biphenyl-4-yl)-(E)-vinyl]-imidazol-1-yl}-benzoic acid (49 mg, 80% yield).

LCMS: 605 (M+H)^{+ 1}H NMR (DMSO, 400 MHz): δ 3.05 (s, 3H), 6.49 (d, 2H), 6.62 (d, 2H), 6.69 (s, 1H), 6.92 (d, 1H), 7.06 (d, 2H), 7.21 (s, 1H), 7.40-7.54 (m, 3H), 7.56-7.66 (m, 2H), 8.13 (d, 2H), 8.30 (d, 1H), 9.63 (s, 1H), 9.87 (s, 1H) ppm.

By analagous methods to those used to prepare Example 296, the following compounds were synthesized:

| Example | Name | LC/MS (m/z) |
|---|---|---|
| 297 | 4-(4-(2,4-Dichloro-phenyl)-2-{2-[3′-(2,2,2- | 673 (M + H)⁺ |
| | trifluoro-ethanesulfonylamino)-biphenyl-4- | |
| | yl]-(E)-vinyl}-imidazol-1-yl)-benzoic | |
| | acid | |
| 298 | 4-(4-(2,4-Dichloro-phenyl)-2-{2-[3′- | 633 (M + H)⁺ |
| | (propane-2-sulfonylamino)-biphenyl-4-yl]- | |
| | (E)-vinyl}-imidazol-1-yl)-benzoic acid | |

### Example 299

2-[2-(4-Bromo-phenyl)-(E)-vinyl]-4-(2,4-dichloro-phenyl)-1H-imidazole (4.0 g, 10 mmol) was reacted with methyl 3-fluoro benzoate (2.3 g, 15 mmol) following general procedure I to give 3-[2-[2-(4-Bromo-phenyl)-(E)-vinyl]-4-(2,4-dichloro-phenyl)-imidazol-1-yl]-benzoic acid methyl ester (3.7 g, 70%).

3-[2-[2-(4-Bromo-phenyl)-(E)-vinyl]-4-(2,4-dichloro-phenyl)-imidazol-1-yl]-benzoic acid methyl ester (265 mg, 0.5 mmol) was coupled with 3-(methanesulfonyl)-phenyl boronic acid (110 mg, 0.55 mmol) following general procedure B to give 3-{4-(2,4-Dichloro-phenyl)-2-[2-(3′-methanesulfonyl-biphenyl-4-yl)-(E)-vinyl]-imidazol-1-yl}-benzoic acid methyl ester (212 mg, 70%).

3-{4-(2,4-Dichloro-phenyl)-2-[2-(3′-methanesulfonyl-biphenyl-4-yl)-(E)-vinyl]-imidazol-1-yl}-benzoic acid methyl ester (60 mg, 0.1 mmol) was hydrolyzed according to general procedure F to give 3-{4-(2,4-Dichloro-phenyl)-2-[2-(3′-methanesulfonyl-biphenyl-4-yl)-(E)-vinyl]-imidazol-1-yl}-benzoic acid (47 mg, 81% yield).

LCMS: 590 (M+H)^{+ 1}H NMR (DMSO, 400 MHz): δ 3.28 (s, 3H), 6.96 (s, 1H), 7.02 (s, 1H), 7.53 (d, 2H), 7.57 (d, 2H), 7.66-7.79 (m, 4H), 7.93 (d, 1H), 8.09-8.18 (m, 3H), 8.22 (d, 2H), 8.27 (s, 1H), 8.32 (s, 1H) ppm.

### Example 300

2-[2-(4-Bromo-phenyl)-(E)-vinyl]-4-(2,4-dichloro-phenyl)-1H-imidazole (4.0 g, 10 mmol) was reacted with methyl 4-(bromomethy) benzoate (3.5 g, 15 mmol) following general procedure E to give 4-[2-[2-(4-bromo-phenyl)-(E)-vinyl]-4-(2,4-dichloro-phenyl)-imidazol-1-ylmethyl]-benzoic acid methyl ester (4.1 g, 75%).

4-[2-[2-(4-Bromo-phenyl)-(E)-vinyl]-4-(2,4-dichloro-phenyl)-imidazol-1-ylmethyl]-benzoic acid methyl ester (55 mg, 0.1 mmol) was hydrolyzed following general procedure F to give 4-[2-[2-(4-bromo-phenyl)-(E)-vinyl]-4-(2,4-dichloro-phenyl)-imidazol-1-ylmethyl]-benzoic acid (46 mg, 85%).

LCMS: m/z 529 (M+H)⁺

### Example 301

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-hydroxy phenyl boronic acid (76 mg, 0.55 mmol) following general procedure B to give 4-{4-(2,4-dichloro-phenyl)-2-[2-(4′-hydroxy-biphenyl-4-yl)-(E)-vinyl]-imidazol-1-ylmethyl}-benzoic acid methyl ester (171 mg, 67%).

4-{4-(2,4-dichloro-phenyl)-2-[2-(4′-hydroxy-biphenyl-4-yl)-(E)-vinyl]-imidazol-1-ylmethyl}-benzoic acid methyl ester (139 mg, 0.25 mmol) was treated with 4-fluoronitrobenzene (39 mg, 0.27 mmol) according to general procedure I to give 4-[2-{2-[4′-(4-nitro-phenoxy)-biphenyl-4-yl]-(E)-vinyl}-4-(2,4-dichloro-phenyl)-imidazol-1-ylmethyl]-benzoic acid methyl ester (119 mg, 70%).

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (68 mg, 0.1 mmol) was hydrolyzed following general procedure F to give 4-[2-{2-[4′-(4-nitro-phenoxy)-biphenyl-4-yl]-(E)-vinyl}-4-(2,4-dichloro-phenyl)-imidazol-1-ylmethyl]-benzoic acid (49 mg, 74%).

LCMS: m/z 663 (M+H)⁺

### Example 302

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (170 mg, 0.25 mmol) was reduced to amino compound (121 mg, 74%) following general procedure K and was treated with methanesulfonyl chloride (23 mg, 0.2 mmol) to give 4-(4-(2-4-dichloro-phenyl)-2-{2-[4′-(4-methanesulfonylamino-phenoxy)-biphenyl-4-yl]-(E)-vinyl}-imidazol-1-yl-methyl)-benzoic acid methyl ester (101 mg, 75%).

4-(4-(2-4-Dichloro-phenyl)-2-{2-[4′-(4-methanesulfonylamino-phenoxy)-biphenyl-4-yl]-(E)-vinyl}-imidazol-1-yl-methyl)-benzoic acid methyl ester (73 mg, 0.1 mmol) was hydrolyzed following general procedure F to give 4-(4-(2-4-dichloro-phenyl)-2-{2-[4′-(4-methanesulfonylamino-phenoxy)-biphenyl-4-yl]-(E)-vinyl}-imidazol-1-yl-methyl)-benzoic acid (56 mg, 78%).

LCMS: m/z 711 (M+H)⁺

### Example 303

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-(tert butyl)-phenyl boronic acid (98 mg, 0.55 mmol) following general procedure B to give 4-[2-[2-(4′-tert-butyl-biphenyl-4-yl)-(E)-vinyl]-4-(2,4-dichloro-phenyl)-imidazol-1-ylmethyl]-benzoic acid methyl ester (207 mg, 69%).

4-[2-[2-(4′-tert-Butyl-biphenyl-4-yl)-(E)-vinyl]-4-(2,4-dichloro-phenyl)-imidazol-1-ylmethyl]-benzoic acid methyl ester (60 mg, 0.1 mmol) was hydrolyzed according to general procedure F to give 4-[2-[2-(4′-tert-butyl-biphenyl-4-yl)-(E)-vinyl]-4-(2,4-dichloro-phenyl)-imidazol-1-ylmethyl]-benzoic acid (45 mg, 77%).

LCMS: 582 (M+H)^{+ 1}H NMR (DMSO, 400 MHz): δ 1.28 (s, 9H), 5.84 (s, 2H), 7.47-7.51 (m, 2H), 7.56 (s, 1H), 7.58-7.64 (m, 3H), 7.71-7.88 (m, 4H), 7.90-7.99 (m, 4H), 8.14-8.19 (m, 3H), 8.32 (s, 1H) ppm.

By analagous methods to those used to prepare Example 303, the following compounds were synthesized:

| Example | Name | LC/MS (m/z) |
|---|---|---|
| 304 | 4-[2-[2-(4′-trifluoromethyl-biphenyl-4-yl)- | 594 (M + H)⁺ |
| | (E)-vinyl]-4-(2,4-dichloro-phenyl)-imidazol- | |
| | 1-ylmethyl]-benzoic acid | |
| 305 | 4-{4-(2,4-dichloro-phenyl)-2-[2-(3′- | 604 (M + H)⁺ |
| | methanesulfonyl-biphenyl-4-yl)-(E)-vinyl]- | |
| | imidazol-1-ylmethyl}-benzoic acid | |
| 306 | 4-[2-[2-(3′-trifluoromethoxy-biphenyl-4-yl)- | 510 (M + H)⁺ |
| | (E)-vinyl]-4-(2,4-dichloro-phenyl)-imidazol- | |
| | 1-ylmethyl]-benzoic acid | |

### Example 307

2-[2-(4-Bromo-phenyl)-(E)-vinyl]-4-(2,4-dichloro-phenyl)-1H-imidazole (2.0 g, 5 mmol) was reacted with methyl 4-(bromomethy) phenyl acetic acid methyl ester (1.5 g, 6 mmol) following general procedure E to give {4-4-[2-[2-(4-bromo-phenyl)-(E)-vinyl]-4-(2,4-dichloro-phenyl)-imidazol-1-ylmethyl]-phenyl}-acetic acid methyl ester (1.7 g, 60%).

{4-[2-[2-(4-Bromo-phenyl)-(E)-vinyl]-4-(2,4-dichloro-phenyl)-imidazol-1-ylmethyl]-phenyl}-acetic acid methyl ester (272 mg, 0.5 mmol) was coupled with 3-(trifluoromethyl)-phenyl boronic acid (104 mg, 0.55 mmol) following general procedure B to give (4-{4-(2,4-dichloro-phenyl)-2-[2-(3′-trifluoromethyl-biphenyl-4-yl)-(E)-vinyl]-imidazol-1-ylmethyl}-phenyl) -acetic acid methyl ester (198 mg, 65%).

(4-{4-(2,4-Dichloro-phenyl)-2-[2-(3′-trifluoromethyl-biphenyl-4-yl)-(E)-vinyl]-imidazol-1-ylmethyl}-phenyl) -acetic acid methyl ester (63 mg, 0.1 mmol) was hydrolyzed following general procedure F to give (4-{4-(2,4-dichloro-phenyl)-2-[2-(3′-trifluoromethyl-biphenyl-4-yl)-(E)-vinyl]imidazol-1-ylmethyl}-phenyl) -acetic acid (46 mg, 75%).

LCMS: 608 (M+H)⁺

### Example 308

(4-{4-(2,4-dichloro-phenyl)-2-[2-(3′-methanesulfony-biphenyl-4-yl)-(E)-vinyl]-imidazol-1-ylmethyl}-phenyl)-acetic acid was prepared by analagous methods to those used to prepare Example 307. LCMS: 618 (M+H)⁺

### Example 309

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 5-chlorothiphene-2-boronic acid (90 mg, 0.55 mmol) following general procedure B to give 4-[2-{2-[4-(5-chloro-thiophen-2-yl)-phenyl]-(E)-vinyl}-4-(2,4-dichloro-phenyl)-imidazol-1-ylmethyl]-benzoic acid methyl ester (199 mg, 68%).

4-[2-{2-[4-(5-Chloro-thiophen-2-yl)-phenyl]-(E)-vinyl}-4-(2,4-dichloro-phenyl)-imidazol-1-ylmethyl]-benzoic acid methyl ester (58 mg, 0.1 mmol) was hydrolyzed according to general procedure F to give 4-[2-{2-[4-(5-chloro-thiophen-2-yl)-phenyl]-(E)-vinyl}-4-(2,4-dichloro-phenyl)-imidazol-1-ylmethyl]-benzoic acid (42 mg, 75%).

LCMS: 566 (M+H)^{+ 1}H NMR (DMSO, 400 MHz): δ 5.43 (s, 2H), 6.57 (d, 1H), 6.70 (d, 1H), 7.02-7.17 (m, 4H), 7.18-7.27 (m, 2H), 7.39-7.49 (m, 2H), 7.50-7.64 (m, 2H), 7.75 (d, 1H), 8.03-8.09 (m, 2H), 8.22 (d, 1H) ppm.

### Example 310

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-isopropylthiophenyl boronic acid (108 mg, 0.55 mmol) following general procedure B to give 4-{4-(2,4-dichloro-phenyl)-2-[2-(4′-isopropylsulfanyl-biphenyl-4-yl)-(E)-vinyl]-imidazol-1-ylmethyl}-benzoic acid methyl ester (211 mg, 68%).

4-{4-(2,4-Dichloro-phenyl)-2-[2-(4′-isopropylsulfanyl-biphenyl-4-yl)-(E)-vinyl]-imidazol-1-ylmethyl}-benzoic acid methyl ester (62 mg, 0.1 mmol) was hydrolyzed according to general procedure F to give 4-{4-(2,4-dichloro-phenyl)-2-[2-(4′-isopropylsulfanyl-biphenyl-4-yl)-(E)-vinyl]-imidazol-1-ylmethyl}-benzoic acid (50 mg, 80%).

LCMS: 600 (M+H)^{+ 1}H NMR (DMSO, 400 MHz): δ 1.26 (d, 6H), 3.48 (m, 1H), 5.43 (s, 2H), 6.54 (d, 1H), 6.73 (d, 1H), 7.02-7.17 (m, 4H), 7.19-7.28 (m, 2H), 7.42-7.49 (m, 2H), 7.50-7.68 (m, 4H), 7.70 (d, 1H), 8.04-8.11 (m, 2H), 8.26 (d, 1H) ppm.

### Example 311

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 1-(tert butoxy carbonyl) 5-methoxy-1H-indol-2-yl-boronic acid (160 mg, 0.55 mmol) following general procedure B to give 2-(4-{2-[4-(2,4-dichloro-phenyl)-1-(4-methoxycarbonyl-benzyl)-1H-imidazol-2-yl]-(E)-vinyl}-phenyl)-5-methoxy-indole-1-carboxylic acid tert-butyl ester (217 mg, 61%).

2-(4-{2-[4-(2,4-Dichloro-phenyl)-1-(4-methoxycarbonyl-benzyl)-1H-imidazol-2-yl]-(E)-vinyl}-phenyl)-5-methoxy-indole-1-carboxylic acid tert-butyl ester (70 mg, 0.1 mmol) was hydrolyzed according to general procedure F to give 2-(4-{2-[1-(4-carboxy-benzyl)-4-(2,4-dichloro-phenyl)-1H-imidazol-2-yl]-(E)-vinyl}-phenyl)-5-methoxy-indole-1-carboxylic acid tert-butyl ester (56 mg, 82%).

LCMS: 696 (M+H)^{+ 1}H NMR (DMSO, 400 MHz): δ 1.27 (s, 9H), 3.78 (s, 3H), 5.64 (s, 2H), 6.85 (s, 1H), 6.95 (d, 1H), 7.12 (s, 1H), 7.34 (d, 2H), 7.37 (d, 2H), 7.43-7.55 (m, 4H), 7.64 (s, 1H), 7.72 (d, 2H), 7.91-7.97 (m, 2H), 8.12 (s, 1H), 8.30 (d, 1H) ppm.

### Example 312

2-(4-{2-[1-(4-Carboxy-benzyl)-4-(2,4-dichloro-phenyl)-1H-imidazol-2-yl]-(E)-vinyl}-phenyl)-5-methoxy-indole-1-carboxylic acid tert-butyl ester (70 mg, 0.1 mmol was treated with 2N HCl in dioxane following general procedure to give 4-(4-(2,4-Dichloro-phenyl)-2-{2-[4-(5-methoxy-1H-indol-2-yl)-phenyl]-(E)-vinyl}-imidazol-1-ylmethyl)-benzoic acid (47 mg, 79%).

LCMS: 596 (M+H)⁺

### Example 313

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 3-(morpholino)-phenyl boronic acid Hcl (146 mg, 0.6 mmol) following general procedure B to give 4-{4-(2,4-dichloro-phenyl)-2-[2-(3′-morpholin-4-yl-biphenyl-4-yl)-(E)-vinyl]-imidazol-1-ylmethyl}-benzoic acid methyl ester (207 mg, 66%).

4-{4-(2,4-Dichloro-phenyl)-2-[2-(3′-morpholin-4-yl-biphenyl-4-yl)-(E)-vinyl]-imidazol-1-ylmethyl}-benzoic acid methyl ester (63 mg, 0.1 mmol) was hydrolyzed according to general procedure F to give 4-{4-(2,4-Dichloro-phenyl)-2-[2-(3′-morpholin-4-yl-biphenyl-4-yl)-(E)-vinyl]-imidazol-1-ylmethyl}-benzoic acid (49 mg, 80%).

LCMS: 611 (M+H)⁺

### Example 314

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 2-methoxy-5-pyridine-boronic acid (84 mg, 0.55 mmol) following general procedure B to give 4-{4-(2,4-Dichloro-phenyl)-2-[2-[4-(6-methoxy-pyridin-3-yl)-phenyl]-(E)-vinyl}-imidazol-1-ylmethyl)-benzoic acid methyl ester (201 mg, 70%).

4-{4-(2,4-Dichloro-phenyl)-2-[2-[4-(6-methoxy-pyridin-3-yl)-phenyl]-(E)-vinyl}-imidazol-1-ylmethyl)-benzoic acid methyl ester (60 mg, 0.1 mmol) was hydrolyzed according to general procedure F to give 4-{4-(2,4-Dichloro-phenyl)-2-[2-[4-(6-methoxy-pyridin-3-yl)-phenyl]-(E)-vinyl}-imidazol-1-ylmethyl)-benzoic acid (47 mg, 81% yield).

LCMS: 557 (M+H)⁺

### Example 315

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with pyridine boronic acid (68 mg, 0.55 mmol) following general procedure B to give 4-{4-(2,4-Dichloro-phenyl)-2-[2-[4-(pyridin-3-yl)-phenyl]-(E)-vinyl}-imidazol-1-ylmethyl)-benzoic acid methyl ester (197 mg, 72%).

4-{4-(2,4-Dichloro-phenyl)-2-[2-[4-(pyridin-3-yl)-phenyl]-(E)-vinyl}-imidazol-1-ylmethyl)-benzoic acid methyl ester (55 mg, 0.1 mmol) was hydrolyzed according to general procedure F to give 4-{4-(2,4-Dichloro-phenyl)-2-[2-[4-(pyridin-3-yl)-phenyl]-(E)-vinyl}-imidazol-1-ylmethyl)-benzoic acid (41 mg, 78% yield).

LCMS: 527 (M+H)⁺

### Example 316

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with pyrimidine boronic acid (69 mg, 0.55 mmol) following general procedure B to give 4-{4-(2,4-dichloro-phenyl)-2-[2-[4-(pyrimidin-3-yl)-phenyl]-(E)-vinyl}-imidazol-1-ylmethyl)-benzoic acid methyl ester (167 mg, 62%).

4-{4-(2,4-Dichloro-phenyl)-2-[2-[4-(pyrimidin-3-yl)-phenyl]-(E)-vinyl}-imidazol-1-ylmethyl)-benzoic acid methyl ester (55 mg, 0.1 mmol) was hydrolyzed according to general procedure F to give 4-{4-(2,4-Dichloro-phenyl)-2-[2-[4-(pyrimidin-3-yl)-phenyl]-(E)-vinyl}-imidazol-1-ylmethyl)-benzoic acid (38 mg, 71% yield).

LCMS: 528 (M+H)⁺

### Example 317

4-{4-(2,4-Dichloro-phenyl)-2-[2-[4-(6-methoxy-pyridin-3-yl)-phenyl]-(E)-vinyl}-imidazol-1-ylmethyl)-benzoic acid methyl ester (60 mg, 0.1 mmol) was reacted with boron tribromide (50 mg, 0.2 mmol) following general procedure C to give 4-{4-(2,4-Dichloro-phenyl)-2-[2-[4-(6-hydroxy-pyridin-3-yl)-phenyl]-(E)-vinyl}-imidazol-1-ylmethyl)-benzoic acid (31 mg, 55% yield)

LCMS: 543 (M+H)⁺

### Example 318

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-ethylsulfinyl phenyl boronic acid (109 mg, 0.55 mmol) following general procedure B to give 4-{4-(2,4-Dichloro-phenyl)-2-[2-(4′-ethanesulfinyl-biphenyl-4-yl)-(E)-vinyl]-imidazol-1-ylmethyl}-benzoic acid methyl ester (201 mg, 65%).

4-{4-(2,4-Dichloro-phenyl)-2-[2-(4′-ethanesulfinyl-biphenyl-4-yl)-(E)-vinyl]-imidazol-1-ylmethyl}-benzoic acid methyl ester (62 mg, 0.1 mmol) was hydrolyzed according to general procedure F to give 4-{4-(2,4-Dichloro-phenyl)-2-[2-(4′-ethanesulfinyl-biphenyl-4-yl)-(E)-vinyl]-imidazol-1-ylmethyl}-benzoic acid (50 mg, 80%).

LCMS: 602 (M+H)^{+ 1}H NMR (DMSO, 400 MHz): δ 1.07 (t, 3H), 2.79 (m, 1H), 3.06 (m, 1H), 5.65 (s, 2H), 7.35-7.40 (m, 4H), 7.51 (d, 1H), 7.53 (s, 1H), 7.57 (d, 1H), 7.61-7.78 (m, 5H), 7.90-7.95 (m, 4H), 8.11 (s, 1H), 8.30 (d, 1H) ppm.

### Example 319

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 5-acetylthiophene-2-boronic acid (94 mg, 0.55 mmol) following general procedure B to give 4-[2-{2-[4-(5-acetyl-thiophen-2-yl-phenyl]-(E)-vinyl}-4-(2,4-dichloro-phenyl)-imidazol-1-ylmethyl]-benzoic acid methyl ester (198 mg, 68%).

4-[2-{2-[4-(5-Acetyl-thiophen-2-yl-phenyl]-(E)-vinyl}-4-(2,4-dichloro-phenyl)-imidazol-1-ylmethyl]-benzoic acid methyl ester (58 mg, 0.1 mmol) was hydrolyzed according to general procedure F to give 4-[2-{2-[4-(5-acetyl-thiophen-2-yl-phenyl]-(E)-vinyl}-4-(2,4-dichloro-phenyl)-imidazol-1-ylmethyl]-benzoic acid (47 mg, 82% yield).

LCMS: 574 (M+H)^{+ 1}H NMR (DMSO, 400 MHz): δ 5.70 (s, 2H), 7.39-7.43 (m, 4H), 7.47 (s, 1H), 7.54 (d, 1H), 7.70-7.82 (m, 5H), 7.93-7.96 (m, 4H), 8.18-8.22 (m, 4H) ppm.

### Example 320

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 3-amino-phenyl boronic acid (75 mg, 0.55 mmol) following general procedure B to give 4-[2-[2-(3′-amino-biphenyl-4-yl)-(E)-vinyl]-4-(2,4-dichloro-phenyl)-imidazol-1-ylmethyl]-benzoic acid methyl ester methyl ester (201 mg, 72%).

4-[2-[2-(3′-amino-biphenyl-4-yl)-(E)-vinyl]-4-(2,4-dichloro-phenyl)-imidazol-1-ylmethyl]-benzoic acid methyl (185 mg, 0.33 mmol) was reacted with 2,2,2,-trifluoroethanesulfonyl chloride (64 mg, 0.35 mmol) following general procedure L to give 4-(4-(2,4-dichloro-phenyl)-2-{2-[3′-(2,2,2-trifluoro-ethanesulfonylamino)-biphenyl-4-yl]-(E)-vinyl}-imidazol-1-ylmethyl)-benzoic acid methyl ester (179 mg, 76%).

4-(4-(2,4-Dichloro-phenyl)-2-{2-[3′-(2,2,2-trifluoro-ethanesulfonylamino)-biphenyl-4-yl]-(E)-vinyl}-imidazol-1-ylmethyl)-benzoic acid methyl ester (70 mg, 0.1 mmol) was hydrolyzed according to general procedure F to give 4-(4-(2,4-dichloro-phenyl)-2-{2-[3′-(2,2,2-trifluoro-ethanesulfonylamino)-biphenyl-4-yl]-(E)-vinyl}-imidazol-1-yl)benzoic acid (51 mg, 75%).

LCMS: 687 (M+H)^{+ 1}H NMR (DMSO, 400 MHz): δ 4.49 (m 2H), 5.63 (S, 2H), 7.18 (d, 1H), 7.34-7.59 (m, 6H), 7.60-7.65 (m, 4H), 7.76 (d, 2H), 7.92 (d, 2H), 8.11-8.15 (m, 2H), 8.28 (d, 2H) ppm.

### Example 321

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 3-methoxycarbonyl-phenyl boronic acid (99 mg, 0.55 mmol) following general procedure B to give 4′-{2-[4-(2,4-dichloro-phenyl)-1-(4-methoxycarbonyl-benzyl)-1H-imidazol-2-yl]-(E)-vinyl}-biphenyl-3-carboxylic acid methyl ester (211 mg, 70%).

4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester (60 mg, 0.1 mmol) was hydrolyzed according to general procedure F to give 4′-{2-[4-(2,4-dichloro-phenyl)-1-(4-methoxycarbonyl-benzyl)-1H-imidazol-2-yl]-(E)-vinyl}-biphenyl-3-carboxylic acid (39 mg, 68% yield).

LCMS: 570 (M+H)⁺

### Example 322

4-[2-[2-(3′-Amino-biphenyl-4-yl)-(E)-vinyl]-4-(2,4-dichloro-phenyl)-imidazol-1-ylmethyl]-benzoic acid methyl (185 mg, 0.33 mmol) was reacted with methylbromo acetate (4 mg, 0.35 mmol) following general procedure L to give 4-(4-(2,4-dichloro-phenyl)-2-{2-[3′-(methoxycarbonylmethyl-amino)-biphenyl-4-yl]-(E)-vinyl}-imidazol-1-ylmethyl)-benzoic acid methyl ester (161 mg, 77%).

4-(4-(2,4-Dichloro-phenyl)-2-{2-[3′-(1,1,4-trioxo-1-[1,2,5]-thiadiazolidin-2-yl)-biphenyl-4-yl]-(E)-vinyl}-imidazol-1-ylmethyl)-benzoic acid methyl ester (97 mg, 57%) was prepared from 4-(4-(2,4-dichloro-phenyl)-2-{2-[3′-(methoxycarbonylmethyl-amino)-biphenyl-4-yl]-(E)-vinyl}-imidazol-1-ylmethyl)-benzoic acid methyl ester (157 mg, 0.25 mmol) following general procedure Y3.

4-(4-(2,4-Dichloro-phenyl)-2-{2-[3′-(1,1,4-trioxo-1-[1,2,5]-thiadiazolidin-2-yl)-biphenyl-4-yl]-(E)-vinyl}-imidazol-1-ylmethyl)-benzoic acid methyl ester (68 mg, 0.1 mmol) was hydrolyzed according to general procedure F to give 4-(4-(2,4-Dichloro-phenyl)-2-{2-[3′-(1,1,4-trioxo-1-[1,2,5]-thiadiazolidin-2-yl)-biphenyl-4-yl]-(E)-vinyl}-imidazol-1-ylmethyl)-benzoic acid (51 mg, 78% yield).

LCMS: 660 (M+H)⁺.

### Example 323

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-amino-phenyl boronic acid (75 mg, 0.55 mmol) following general procedure B to give 4-[2-[2-(4′-Amino-biphenyl-4-yl)-(E)-vinyl]-4-(2,4-dichloro-phenyl)-imidazol-1-ylmethyl]-benzoic acid methyl ester methyl ester (211 mg, 75%).

4-[2-[2-(4′-Amino-biphenyl-4-yl)-(E)-vinyl]-4-(2,4-dichloro-phenyl)-imidazol-1-ylmethyl]-benzoic acid methyl ester (185 mg, 0.33 mmol) was reacted with 2,2,2,-trifluoroethanesulfonyl chloride (64 mg, 0.35 mmol) following general procedure L to give 4-(4-(2,4-Dichloro-phenyl)-2-{2-[4′-(2,2 ,2-trifluoro-ethanesulfonylamino)-biphenyl-4-yl]-(E)-vinyl}-imidazol-1-ylmethyl)-benzoic acid methyl ester (181 mg, 78%).

4-(4-(2,4-Dichloro-phenyl)-2-{2-[4′-(2,2,2-trifluoro-ethanesulfonylamino)-biphenyl-4-yl]-(E)-vinyl}-imidazol-1-ylmethyl)-benzoic acid methyl ester (70 mg, 0.1 mmol) was hydrolyzed according to general procedure F to give 4-(4-(2,4-dichloro-phenyl)-2-{2-[4′-(2,2,2-trifluoro-ethanesulfonylamino)-biphenyl-4-yl]-(E)-vinyl}-imidazol-1-yl)-benzoic acid (54 mg, 79%).

LCMS: 687 (M+H)⁺; ¹H NMR (DMSO, 400 MHz): δ 4.55 (m, 2H), 5.71 (s, 2H), 7.30 (d, 2H), 7.41 (d, 2H), 7.58-7.64 (m, 6H), 7.72-7.78 (m, 4H), 7.93 (d, 2H), 8.01 (d, 1H), 8.21 (d, 1H), 10.61 (s, 1H) ppm.

### Example 324

4-{4-(2,4-dichloro-phenyl)-2-[2-(4′-isopropoxycarbonylamino-biphenyl-4-yl)-(E)-vinyl]-imidazol-1-ylmethyl}-benzoic acid prepared by analagous methods to those used to prepare Example 323. LCMS: 627 (M+H)⁺

### Example 325

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 4-N-boc-amino-3-methoxy phenylboronic acid (148 mg, 0.55 mmol) following general procedure B to give

4-[2-[2-(4′-tert-butoxycarbonylamino-3′-methoxy-biphenyl-4-yl)-(E)-vinyl]-4-(2,4-dichloro-phenyl)-imidazol-1-ylmethyl]-benzoic acid methyl ester (214 mg, 62%).

4-[2-[2-(4′-tert-Butoxycarbonylamino-3′-methoxy-biphenyl-4-yl)-(E)-vinyl]-4-(2,4-dichloro-phenyl)-imidazol-1-ylmethyl]-benzoic acid methyl ester (69 mg, 0.1 mmol) was hydrolyzed according to general procedure F to give 4-[2-[2-(4′-tert-butoxycarbonylamino-3′-methoxy-biphenyl-4-yl)-(E)-vinyl]-4-(2,4-dichloro-phenyl)-imidazol-1-ylmethyl]-benzoic acid (55 mg, 82%).

LCMS: 671 (M+H)^{+ 1}H NMR (DMSO, 400 MHz): δ 1.44 (s, 9H), 3.86 (s. 3H), 5.69 (s, 2H), 6.99 (d, 2H), 7.12 (d, 1H), 7.19 (d, 2H), 7.31 (d, 2H), 7.46 (d, 1H), 7.56-7.73 (m, 4H), 7.91 (d, 2H), 8.06 (s, 1H), 8.18 (d, 2H) ppm

### Example 326

4-[2-[2-(4′-tert-Butoxycarbonylamino-3′-methoxy-biphenyl-4-yl)-(E)-vinyl]-4-(2,4-dichloro-phenyl)-imidazol-1-ylmethyl]-benzoic acid (67 mg, 0.1 mmol) was treated with 2N HCl in dioxane following general procedure O to give 4-[2-[2-(4′-Amino-3′-methoxy-biphenyl-4-yl)-(E)-vinyl]-4-(2,4-dichloro-phenyl)-imidazol-1-ylmethyl]-benzoic acid (41 mg, 72%).

LCMS: 571 (M+H)⁺

### Example 327

4-[2-[2-(4′-Amino-3′-methoxy-biphenyl-4-yl)-(E)-vinyl]-4-(2,4-dichloro-phenyl)-imidazol-1-ylmethyl]-benzoic acid methyl ester (195 mg, 0.33 mmol) was reacted with 2,2,2,-trifluoroethanesulfonyl chloride (64 mg, 0.35 mmol) following general procedure L to give 4-(4-(2,4-Dichloro-phenyl)-2-{2-[3′-methoxy-4′-(2,2,2-trifluoro-ethanesulfonyl-amino)-biphenyl-4-yl]-(E)-vinyl}-imidazol-1-ylmethyl)-benzoic acid methyl ester (189 mg, 77%).

4-(4-(2,4-Dichloro-phenyl)-2-{2-[3′-methoxy-4′-(2,2,2-trifluoro-ethanesulfonyl-amino)-biphenyl-4-yl]-(E)-vinyl}-imidazol-1-ylmethyl)-benzoic acid methyl ester (74 mg, 0.1 mmol) was hydrolyzed according to general procedure F to give 4-(4-(2,4-Dichloro-phenyl)-2-{2-[3′-methoxy-4′-(2,2,2-trifluoro-ethanesulfonylamino)-biphenyl-4-yl]-(E)-vinyl}-imidazol-1-ylmethyl)-benzoic acid (71 mg, 84%).

LCMS: 717 (M+H)^{+ 1}H NMR (DMSO, 400 MHz): δ 3.92 (s, 3H), 4.32 (m, 2H), 5.64 (s, 2H), 7.25-7.36 (m, 4H), 7.50-7.59 (m, 4H), 7.65-7.76 (m, 4H), 7.89 (d, 2H), 7.94 (d, 1H), 8.06 (s, 1H), 8.15 (d, 1H), 8.29 (d, 1H) ppm.

By analagous methods to those used to prepare Example 327, the following compounds were synthesized:

| Example | Name | LC/MS (m/z) |
|---|---|---|
| 328 | 4-{4-(2,4-Dichloro-phenyl)-2-[2-(4′-methanesulfonylamino-3′- | 649 (M + H)⁺ |
| | methoxy-biphenyl-4-yl)-(E)-vinyl]-imidazol-1-ylmethyl}- | |
| | benzoic acid | |
| 329 | 4-{4-(2,4-Dichloro-phenyl)-2-[2-(4′-ethoxycarbonylamino-3′- | 643 (M + H)⁺ |
| | methoxy-biphenyl-4-yl)-(E)-vinyl]-imidazol-1-ylmethyl}- | |
| | benzoic acid | |
| 330 | 4-(4-(2,4-dichloro-phenyl)-2-{2-[3′-methoxy-4′-(2-methoxy- | 673 (M + H)⁺ |
| | ethoxycarbonylamino)-biphenyl-4-yl]-(E)-vinyl}-imidazol-1- | |
| | ylmethyl)-benzoic acid | |
| 331 | 4-{4-(2,4-dichloro-phenyl)-2-[2-(4′-isobutoxycarbonylamino- | 671 (M + H)⁺ |
| | 3′-methoxy-biphenyl-4-yl)-(E)-vinyl]-imidazol-1-ylmethyl}- | |
| | benzoic acid | |
| 332 | 4-{4-(2,4-Dichloro-phenyl)-2-[2-(4′- | 657 (M + H)⁺ |
| | isopropoxycarbonylamino-3′-methoxy-biphenyl-4-yl)-(E)- | |
| | vinyl]-imidazol-1-ylmethyl}-benzoic acid | |
| 333 | 4-(4-(2,4-dichloro-phenyl)-2-{2-[4′-(2,2-dimethyl- | 685 (M + H)⁺ |
| | propoxycarbonylamino)-3′-methoxy biphenyl-4-yl]-(E)-vinyl}- | |
| | imidazol-1-ylmethyl)-benzoic acid | |
| 334 | 4-(4-(2,4-Dichloro-phenyl)-2-{2-[3′-methoxy-4′-(2,2,2- | 696 (M + H)⁺ |
| | trifluoro-ethoxycarbonylamino)-biphenyl-4-yl]-(E)-vinyl}- | |
| | imidazol-1-ylmethyl)-benzoic acid | |

### Example 335

4-[2-[2-(4′-Amino-3′-methoxy-biphenyl-4-yl)-(E)-vinyl]-4-(2,4-dichloro-phenyl)-imidazol-1-ylmethyl]-benzoic acid methyl ester (195 mg, 0.33 mmol) was reacted with isovaleric acid (36 mg, 0.35 mmol) following general procedure G to give 4-(4-(2,4-dichloro-phenyl)-2-{2-[3′-methoxy-4′-(3-methyl-butyrylamino)-biphenyl-4-yl]-(E)-vinyl}-imidazol-1-ylmethyl)-benzoic acid methyl ester (177 mg, 79%).

4-(4-(2,4-Dichloro-phenyl)-2-{2-[3′-methoxy-4′-(3-methyl-butyrylamino)-biphenyl-4-yl]-(E)-vinyl}-imidazol-1-ylmethyl)-benzoic acid methyl ester (67 mg, 0.1 mmol) was hydrolyzed according to general procedure F to give 4-(4-(2,4-dichloro-phenyl)-2-{2-[3′-methoxy-4′-(3-methyl-butyrylamino)-biphenyl-4-yl]-(E)-vinyl}-imidazol-1-ylmethyl)-benzoic acid (51 mg, 78%).

LCMS: 671 (M+H)^{+ 1}H NMR (DMSO, 400 MHz): δ 0.93 (d, 6H), 2.05 (m, 2H), 2.27 (m 1H), 3.92 (s, 3H), 5.53 (s, 2H), 7.19-7.26 (m 2H), 7.32 (s, 1H), 7.36 (d, 2H), 7.49-7.57 (m, 2H), 7.63 (d, 2H), 7.7 (d, 2H), 7.84 (d, 2H), 8.01 (d, 1H), 8.07 (s, 1H), 8.28 (d, 1H), 9.11 (s, 1H) ppm.

### Example 336

4-[2-[2-(4′-Amino-3′-methoxy-biphenyl-4-yl)-(E)-vinyl]-4-(2,4-dichloro-phenyl)-imidazol-1-ylmethyl]-benzoic acid methyl ester (195 mg, 0.33 mmol) was reacted with isopropyl isocyanate (34 mg, 0.35 mmol) following general procedure L to give 4-(4-(2,4-dichloro-phenyl)-2-{2-[4′-(3-isopropyl-ureido)-3′-methoxy-biphenyl-4-yl]-(E)-vinyl}-imidazol-1-ylmethyl)-benzoic acid methyl ester (161 mg, 72%).

4-(4-(2,4-Dichloro-phenyl)-2-{2-[4′-(3-isopropyl-ureido)-3′-methoxy-biphenyl-4-yl]-(E)-vinyl}-imidazol-1-ylmethyl)-benzoic acid methyl ester (67 mg, 0.1 mmol) was hydrolyzed according to general procedure F to give 4-(4-(2,4-dichloro-phenyl)-2-{2-[4′-(3-isopropyl-ureido)-3′-methoxy-biphenyl-4-yl]-(E)-vinyl}-imidazol-1-ylmethyl)-benzoic acid (54 mg, 80% yield).

LCMS: 656 (M+H)^{+ 1}H NMR (DMSO, 400 MHz): δ 0.98 (d, 6H), 3.87 (m 1H), 3.93 (s, 3H), 5.64 (s, 2H), 6.81 (d, 1H), 7.20 (d, 1H), 7.26 (d, 2H), 7.31 (s, 1H), 7.35 (d, 2H), 7.50-7.58 (m, 2H), 7.65 (d, 2H), 7.89-7.94 (m, 4H), 8.10 (s, 1H), 8.16 (d, 1H), 8.27 (d, 1H) ppm.

### Example 337

4-[2-[2-(4-Bromo-phenyl)-(E)-vinyl]-4-(2,4-dichloro-phenyl)-imidazol-1-ylmethyl]-benzoic acid methyl ester (272 mg, 0.5 mmol) was coupled with 3-N-Boc-amino-4-methoxy phenylboronic acid (148 mg, 0.55 mmol) following general procedure B to give 4-[2-[2-(3′-tert-Butoxycarbonylamino-4′-methoxy-biphenyl-4-yl)-(E)-vinyl]-4-(2,4-dichloro-phenyl)-imidazol-1-ylmethyl]-benzoic acid methyl ester (216 mg, 64%).

4-[2-[2-(3′-tert-Butoxycarbonylamino-4′-methoxy-biphenyl-4-yl)-(E)-vinyl]-4-(2,4-dichloro-phenyl)-imidazol-1-ylmethyl]-benzoic acid methyl ester (69 mg, 0.1 mmol) was hydrolyzed according to general procedure F to give 4-[2-[2-(3′-tert-Butoxycarbonylamino-4′-methoxy-biphenyl-4-yl)-(E)-vinyl]-4-(2,4-dichloro-phenyl)-imidazol-1-ylmethyl]-benzoic acid (51 mg, 80% yield).

LCMS: 671 (M+H)^{+ 1}H NMR (DMSO, 400 MHz): δ 1.43 (s, 9H), 3.87 (s. 3H), 5.66 (s, 2H), 6.97 (d, 2H), 7.11 (d, 1H), 7.21 (d, 2H), 7.34 (d, 2H), 7.47 (d, 1H), 7.56-7.76 (m, 4H), 7.92 (d, 2H), 8.07 (s, 1H), 8.19 (d, 2H) ppm

### Example 338

4-[2-[2-(3′-tert-Butoxycarbonylamino-4′-methoxy-biphenyl-4-yl)-(E)-vinyl]-4-(2,4-dichloro-phenyl)-imidazol-1-ylmethyl]-benzoic acid (67 mg, 0.1 mmol) was treated with 2N HCl in dioxane following general procedure O to give 4-[2-[2-(3′-Amino-4′-methoxy-biphenyl-4-yl)-(E)-vinyl]-4-(2,4-dichloro-phenyl)-imidazol-1-ylmethyl]-benzoic acid (44 mg, 74% yield).

LCMS: 571 (M+H)⁺

### Example 339

4-[2-[2-(3′-Amino-4′-methoxy-biphenyl-4-yl)-(E)-vinyl]-4-(2,4-dichloro-phenyl)-imidazol-1-ylmethyl]-benzoic acid methyl ester (195 mg, 0.33 mmol) was reacted with methanesulfonyl chloride (40 mg, 0.35 mmol) following general procedure L to give 4-{4-(2,4-dichloro-phenyl)-2-[2-(3′-methanesulfonylamino-4′-methoxy-biphenyl-4-yl)-(E)-vinyl]-imidazol-1-ylmethyl}-benzoic acid methyl ester (172 mg, 77%).

4-{4-(2,4-Dichloro-phenyl)-2-[2-(3′-methanesulfonylamino-4′-methoxy-biphenyl-4-yl)-(E)-vinyl]-imidazol-1-ylmethyl}-benzoic acid methyl ester (66 mg, 0.1 mmol) was hydrolyzed according to general procedure F to give 4-{4-(2,4-dichloro-phenyl)-2-[2-(3′-methanesulfonylamino-4′-methoxy-biphenyl-4-yl)-(E)-vinyl]-imidazol-1-ylmethyl}-benzoic acid (51 mg, 79%).

LCMS: 649 (M+H)⁺

By analagous methods to those used to prepare Example 339, the following compounds were synthesized:

| Example | Name | LC/MS (m/z) |
|---|---|---|
| 340 | 4-(4-(2,4-Dichloro-phenyl)-2-{2-[4′- | 717 (M + H)⁺ |
| | methoxy-3′-(2,2,2-trifluoro-ethane- | |
| | sulfonylamino)-biphenyl-4-yl]-(E)- | |
| | vinyl}-imidazol-1-ylmethyl)-benzoic | |
| | acid | |
| 341 | 4 4-(4-(2,4-dichloro-phenyl)-2-{2-[4′-fluoro- | 665 (M + H)⁺ |
| | 3′-(propane-2-sulfonylamino)-biphenyl | |
| | 4-yl]-(E)-vinyl}-imidazol-1-ylmethyl)- | |
| | benzoic acid | |
| 342 | 4-(4-(2,4-dichloro-phenyl)-2-{2-[3′-(propane- | 647 (M + H)⁺ |
| | 2-sulfonylamino)-biphenyl-4-yl]-(E)-vinyl}- | |
| | imidazol-1-ylmethyl)-benzoic acid | |
| 343 | 4-{4-(2,4-Dichloro-phenyl)-2-[2-(3′- | 627 (M + H)⁺ |
| | isopropoxy-carbonylamino-biphenyl-4-yl)- | |
| | (E)-vinyl]-imidazol-1-ylmethyl}- | |
| | benzoic acid | |
| 344 | 4-{4-(2,4-dichloro-phenyl)-2-[2-(3′-trifluoro- | 673 (M + H)⁺ |
| | methanesulfonylamino-biphenyl-4-yl)-(E)- | |
| | vinyl]-imidazol-1-ylmethyl}-benzoic acid | |

### Example 345

2-[2-(4-Bromo-phenyl)-(E)-vinyl]-4-(2,4-dichloro-phenyl)-1H-imidazole (4.0 g, 10 mmol) was reacted with 4-nitobenzylbromide (3.3 g, 15 mmol) following general procedure E to give nitro compound and was reduced to 4-[2-[2-(4-bromo-phenyl)-(E)-vinyl]-4-(2,4-dichloro-phenyl)-imidazol-1-ylmethyl]-phenylamine (2.9 g, 58%) following general procedure K.

4-[2-[2-(4-Bromo-phenyl)-(E)-vinyl]-4-(2,4-dichloro-phenyl)-imidazol-1-ylmethyl]phenylamine (0.165 mg, 0.33 mmol) was reacted with methane sulfonyl chloride (55 mg, 0.35 mmol) following general procedure L to give N-{4-[2-[2-(4-bromo-phenyl)-(E)-vinyl]-4-(2,4-dichloro-phenyl)-imidazol-1-ylmethyl]-phenyl}-methanesulfonamide (149 mg, 78%).

N-{4-[2-[2-(4-Bromo-phenyl)-(E)-vinyl]-4-(2,4-dichloro-phenyl)-imidazol-1-ylmethyl]phenyl}-methanesulfonamide (144 mg, 0.25 mmol) was coupled with 4-trifluoromethylbenzeneboronic acid (57 mg, 0.3 mmol) following general procedure B to give N-(4-{4-(2,4-Dichloro-phenyl)-2-[2-(4′-trifluoromethyl-biphenyl-4-yl)-(E)-vinyl]-imidazol-1-ylmethyl}phenyl)-methanesulfonamide (112 mg, 70% yield).

LCMS: 643 (M+H)⁺

By analagous methods to those used to prepare Example 345, the following compounds were synthesized:

| Example | Name | LC/MS (m/z) |
|---|---|---|
| 346 | 2,2,2-Trifluoro-ethanesulfonic acid (4-{4- | 711 (M + H)⁺ |
| | (2,4-dichloro-phenyl)-2-[2-(4′-trifluoro- | |
| | methylbiphenyl-4-yl)-(E)-vinyl]- | |
| | imidazol-1-ylmethyl}-phenyl)-amide | |
| 347 | N-(4-{4-(2,4-dichloro-phenyl)-2-[2-(4′- | 697 (M + H)⁺ |
| | trifluoromethyl-biphenyl-4-yl)-(E)-vinyl]- | |
| | imidazol ylmethyl}-phenyl)-trifluoro- | |
| | methanesulfonamide | |

### Example 348

4-[2-[2-(4-Bromo-phenyl)-(E)-vinyl]-4-(2,4-dichloro-phenyl)-imidazol-1-ylmethyl]phenylamine (0.165 mg, 0.33 mmol) was reacted with methyl bromoacetate (54 mg, 0.35 mmol) following general procedure L to give {4-[2-[2-(4-bromo-phenyl)vinyl]-4-(2,4-dichloro-phenyl)-imidazol-1-ylmethyl]-phenylamino}-acetic acid methyl ester (161 mg, 85%).

{4-[2-[2-(4-Bromo-phenyl)-(E)-vinyl]-4-(2,4-dichloro-phenyl)-imidazol-1-ylmethyl]phenylamino}-acetic acid methyl ester (143 mg, 0.25 mmol) was coupled with 4-trifluoromethyl benzeneboronic acid (57 mg, 0.3 mmol) following general procedure B to give (4-{4-(2,4-dichloro-phenyl)-2-[2-(4′-trifluoromethyl-biphenyl-4-yl)-(E)-vinyl]-imidazol-1-ylmethyl}-phenylamino)-acetic acid methyl ester (121 mg, 76%).

(4-{4-(2,4-Dichloro-phenyl)-2-[2-(4′-trifluoromethyl-biphenyl-4-yl)-(E)-vinyl]-imidazol-1-ylmethyl}-phenylamino)-acetic acid methyl ester (64 mg, 0.1 mmol) was hydrolyzed according to general procedure F to give (4-{4-(2,4-dichloro-phenyl)-2-[2-(4′-trifluoromethyl-biphenyl-4-yl)-(E)-vinyl]-imidazol-1-ylmethyl}-phenylamino)-acetic acid (52 mg, 83%).

LCMS: 623 (M+H)^{+ 1}H NMR (DMSO, 400 MHz): δ 3.09 (m, 2H), 5.32 (s, 2H), 6.40 (d, 1H), 6.52 (d, 1H), 7.10 (d, 2H), 7.45-7.55 (m, 4H), 7.63 (s, 1H), 7.78-7.86 (m, 4H), 7.95 (d, 2H), 8.01 (s, 1H), 8.23 (d, 2H), 8.79 (s, 1H) ppm.

### Example 349

(4-{4-(2,4-Dichloro-phenyl)-2-[2-(4′-trifluoromethyl-biphenyl-4-yl)-(E)-vinyl]-imidazol-1-ylmethyl}-phenylamino)-acetic acid methyl ester (210 mg, 0.33 mmol) was reacted with trifluoromethanesulfonic anyhydride (64 mg, 0.35 mmol) following general procedure L to give [(4-{4-(2,4-Dichloro-phenyl)-2-[2-(4′-trifluoromethyl-biphenyl-4-yl)-(E)-vinyl]-imidazol-1-ylmethyl}-phenyl)-trifluoromethanesulfonyl-amino]-acetic acid methyl ester (176 mg, 69%).

[(4-{4-(2,4-Dichloro-phenyl)-2-[2-(4′-trifluoromethyl-biphenyl-4-yl)-(E)-vinyl]-imidazol-1-ylmethyl}-phenyl)-trifluoromethanesulfonyl-amino]-acetic acid methyl ester (77 mg, 0.1 mmol) was hydrolyzed according to general procedure F to give [(4-{4-(2,4-Dichloro-phenyl)-2-[2-(4′-trifluoromethyl-biphenyl-4-yl)-(E)-vinyl]-imidazol-1-ylmethyl}-phenyl)-trifluoromethanesulfonyl-amino]-acetic acid (59 mg, 78% yield).

LCMS: 755 (M+H)^{+ 1}H NMR (DMSO, 400 MHz): δ 2.95 (d, 2H), 5.49 (s, 2H), 6.58 (d, 1H), 7.17 (d, 1H), 7.19 (d, 2H), 7.23 (s, 1H), 7.30-7.49 (m, 2H), 7.51-7.63 (m, 2H), 7.66 (d, 2H), 7.71 (s, 1H), 7.80 (d, 2H), 8.10 (s, 1H), 8.12 (d, 1H), 8.26 (s, 1H), 9.80 (s, 1H) ppm.

### Example 350

[(4-{4-(2,4-Dichloro-phenyl)-2-[2-(4′-trifluoromethyl-biphenyl-4-yl)-(E)-vinyl]-imidazol-1-ylmethyl}-phenyl) -methanesulfonyl-amino]-acetic acid prepared by analagous methods to those used to prepare Example 549. LCMS: 701 (M+H)⁺

### Example 351

(4-{4-(2,4-Dichloro-phenyl)-2-[2-(4′-trifluoromethyl-biphenyl-4-yl)-(E)-vinyl]-imidazol-1-ylmethyl}-phenylamino)-acetic acid methyl ester (210 mg, 0.33 mmol) was reacted with iodomethane (50 mg, 0.35 mmol) following general procedure E to give [(4-{4-(2,4-dichloro-phenyl)-2-[2-(4′-trifluoromethyl-biphenyl-4-yl)-(E)-vinyl]-imidazol-1-ylmethyl}-phenyl) -methyl-amino]-acetic acid methyl ester (179 mg, 83%).

[(4-[(4-{4-(2,4-Dichloro-phenyl)-2-[2-(4′-trifluoromethyl-biphenyl-4-yl)-(E)-vinyl]-imidazol-1-ylmethyl}-phenyl)-methyl-amino]-acetic acid methyl ester (65 mg, 0.1 mmol) was hydrolyzed according to general procedure F to give [(4-{4-(2,4-Dichloro-phenyl)-2-[2-(4′-trifluoromethyl-biphenyl-4-yl)-(E)-vinyl]-imidazol-1-ylmethyl}-phenyl) -methyl-amino]-acetic acid (54 mg, 85% yield).

LCMS: 637 (M+H)^{+ 1}H NMR (DMSO, 400 MHz): δ 2.89 (d, 2H), 3.34 (s, 3H), 5.37 (s, 2H), 6.61 (d, 1H), 7.17 (d, 1H), 7.48-7.56 (m, 4H), 7.60 (d, 2H), 7.81 (d, 2H), 7.84 (d, 2H), 7.94 (d, 2H), 7.99 (d, 2H), 8.07 (d, 1H), 8.24 (d, 1H) ppm.

### Example 352

2-[2-(4-Bromo-phenyl)-(E)-vinyl]-4-(2,4-dichloro-phenyl)-1H-imidazole (4.0 g, 10 mmol) was reacted with 1-4-(bromomethyl)-phenyl-1H-1,2,4,-triazole (3.5 g, 15 mmol) following general procedure E to give 1-{4-[2-[2-(4-bromo-phenyl)-(E)-vinyl]-4-(2,4-dichloro-phenyl)-imidazol-1-ylmethyl]-phenyl}-1H-[1,2,4]-triazole (2.9 g, 52%).

1-{4-[2-[2-(4-Bromo-phenyl)-(E)-vinyl]-4-(2,4-dichloro-phenyl)-imidazol-1-ylmethyl]phenyl}-1H-[1,2,4]-triazole (138 mg, 0.25 mmol) was coupled with 4-trifluoromethylbenzeneboronic acid (57 mg, 0.3 mmol) following general procedure B to give 1-(4-{4-(2,4-dichloro-phenyl)-2-[2-(4′-trifluoromethyl-biphenyl-4-yl)-(E)-vinyl]-imidazol-1-ylmethyl}-phenyl)-1H-[1,2,4]-triazole (119 mg, 77%).

LCMS: 617 (M+H)⁺; ¹H NMR (DMSO, 400 MHz): δ 5.63 (s, 2H), 7.44-7.53 (m, 4H), 7.59 (s, 1H), 7.63 (d, 1H), 7.66 (d, 2H), 7.71-7.82 (m, 4H), 7.85 (d, 2H), 8.01 (d, 2H), 8.15 (s, 1H), 8.21 (s, 1H), 8.26 (d, 1H), 9.24 (s, 1H) ppm.

### Example 353

2,2,2-Trifluoroethanesulfonic acid {4-[2-[2-(4-bromo-phenyl)-(E)-vinyl]-4-(2,4-di-chlorophenyl)-imidazol-1-ylmethyl]-phenyl}-amide (162 mg, 0.25 mmol) was coupled with 3-trifluoromethyl benzeneboronic acid (57 mg, 0.3 mmol) following general procedure B to give 2,2,2-trifluoro-ethanesulfonic acid (4-{4-(2,4-dichloro-phenyl)-2-[2-(3′-trifluoro-methylbiphenyl-4-yl)-(E)-vinyl]-imidazol-1-ylmethyl}-phenyl)-amide (125 mg, 70%).

LCMS: 711 (M+H)^{+ 1}H NMR (DMSO, 400 MHz): δ 4.27 (m, 2H), 5.50 (s, 2H), 7.19 (d, 1H), 7.31 (d, 1H), 7.43-7.56 (m, 4H), 7.63 (d, 2H), 7.68 (d, 2H), 7.73 (s, 1H), 7.78 (d, 2H), 7.82 (d, 2H), 8.03 (s, 1H), 8.25 (d, 1H) ppm.

### Example 354

2-[2-(4-Bromo-phenyl)-(E)-vinyl]-4-(2,4-dichloro-phenyl)-1H-imidazole (4.0 g, 10 mmol) was reacted with 4-bromo (methyl)phenoxyacetic acid methyl ester (3.9 g, 15 mmol) following general procedure E to {4-[2-[2-(4-Bromo-phenyl)-(E)-vinyl]-4-(2,4-dichloro-phenyl)-imidazol-1-ylmethyl]-phenoxy}-acetic acid methyl ester (4.2 g, 48%).

{4-[2-[2-(4-Bromo-phenyl)-(E)-vinyl]-4-(2,4-dichloro-phenyl)-imidazol-1-ylmethyl]phenoxy}-acetic acid methyl ester (143 mg, 0.25 mmol) was coupled with 3-trifluoromethyl benzeneboronic acid (57 mg, 0.3 mmol) following general procedure B to give (4-{4-(2,4-Dichloro-phenyl)-2-[2-(3′-trifluoromethyl-biphenyl-4-yl)-(E)-vinyl]-imidazol-1-ylmethyl}-phenoxy)-acetic acid (101 mg, 63% yield).

(4-{4-(2,4-Dichloro-phenyl)-2-[2-(3′-trifluoromethyl-biphenyl-4-yl)-(E)-vinyl]-imidazol-1-ylmethyl}-phenoxy)-acetic acid methyl ester (64 mg, 0.1 mmol) was hydrolyzed according to general procedure F to give (4-{4-(2,4-Dichloro-phenyl)-2-[2-(3′-trifluoro-methyl-biphenyl-4-yl)-(E)-vinyl]-imidazol-1-ylmethyl}-phenoxy)-acetic acid (50 mg, 80%).

LCMS: 624 (M+H)^{+ 1}H NMR (DMSO, 400 MHz): δ 4.63 (s, 2H), 5.47 (s, 2H), 6.90 (d, 2H), 7.27 (d, 2H), 7.49 (d, 2H), 7.60 (s, 1H), 7.65 (d, 2H), 7.71 (d, 2H), 7.73-7.84 (m, 2H), 8.02 (d, 2H), 8.08 (s, 1H), 8.23 (d, 1H) ppm.

### Example 355

{4-[2-[2-(4-Bromo-phenyl)(E)vinyl]-4-(2,4-dichloro-phenyl)-imidazol-1-ylmethyl]phenylamino}-acetic acid methyl ester (143 mg, 0.25 mmol) was coupled with 3-trifluoromethylbenzeneboronic acid (57 mg, 0.3 mmol) following general procedure B to give (4-{4-(2,4-Dichloro-phenyl)-2-[2-(3′-trifluoromethyl-biphenyl-4-yl)-(E)-vinyl]-imidazol-1-ylmethyl}-phenylamino)-acetic acid methyl ester (111 mg, 70% yield).

5-(4-{4-(2,4-Dichloro-phenyl)-2-[2-(3′-trifluoromethyl-biphenyl-4-yl)-(E)-vinyl]-imidazol-1-ylmethyl}-phenyl)-1,2,5-thiadiazolidin-3-one-1,1-dioxide (31 mg, 52% yield) was prepared according to general procedure Y3 using (4-{4-(2,4-Dichloro-phenyl)-2-[2-(3′-trifluoromethyl-biphenyl-4-yl)-(E)-vinyl]-imidazol-1-ylmethyl}-phenylamino)-acetic acid methyl ester (64 mg, 0.1 mmol).

LCMS: m/z 684 (M+H)⁺; ¹H NMR (DMSO-d₆, 400 MHz): δ 3.94 (s, 2H), 5.45 (s, 2H), 7.05 (d, 2H), 7.27 (d, 2H), 7.45-7.51 (m, 2H), 7.57 (s, 1H), 7.61 (s, 1H), 7.64 (d, 2H), 7.71 (d, 2H), 7.73-7.84 (m, 4H), 8.02 (d, 2H), 8.23 (d, 1H) ppm.

By analagous methods to those used to prepare Example 355, the following compounds were synthesized:

| Example | Name | LC/MS (m/z) |
|---|---|---|
| 356 | 5-{4-[2-[2-(3′,5′-Bis-trifluoromethyl-biphenyl-4-yl)-(E)-vinyl]-4- | 752 (M + H)⁺ |
| | (2,4-dichloro-phenyl) imidazol-1-ylmethyl]-phenyl}-1,2,5- | |
| | thiadiazolidin-3-one-1,1-dioxide | |
| 357 | 5-(4-{4-(2,4-Dichloro-phenyl)-2-[2-(3′-trifluoromethoxy- | 700 (M + H)⁺ |
| | biphenyl-4-yl)-(E)-vinyl]-imidazol-1-ylmethyl}-phenyl)-1,2,5- | |
| | thiadiazolidin-3-one-1,1-dioxide | |
| 358 | 5-(4-{4-(2,4-Dichloro-phenyl)-2-[2-(2′-fluoro-5′-propoxy- | 692 (M + H)⁺ |
| | biphenyl-4-yl)-(E)-vinyl] imidazol-1ylmethyl}-phenyl)-1,2,5- | |
| | thiadiazolidin-3-one-1,1-dioxide | |
| 359 | 5-{4-[2-[2-(3′-Chloro-biphenyl-4-yl)-(E)-vinyl]-4-(2,4-dichloro- | 650 (M + H)⁺ |
| | phenyl)-imidazol-1ylmethyl]-phenyl}-1,2,5-thiadiazolidin-3- | |
| | one-1,1-dioxide | |
| 360 | 5-(4-{4-(2,4-Dichloro-phenyl)-2-[2-(3′-isopropoxy-biphenyl-4- | 674 (M + H)⁺ |
| | yl)-(E)-vinyl]-imidazol-ylmethyl}-phenyl)-1,2,5-thiadiazolidin- | |
| | 3-one-1,1-dioxide | |
| 361 | 5-(4-{4-(2,4-Dichloro-phenyl)-2-[2-(4′-isopropylsulfanyl- | 690 (M + H)⁺ |
| | biphenyl-4-yl)-(E)-vinyl]-imidazol-1 ylmethyl}-phenyl)-1,2,5- | |
| | thiadiazolidin-3-one-1,1-dioxide | |
| 362 | 5-{4-[2-[2-(4′-tert-Butyl-biphenyl-4-yl)-(E)-vinyl]-4-(2,4- | 672 (M + H)⁺ |
| | dichloro-phenyl)-imidazol-1ylmethyl]-phenyl}-1,2,5- | |
| | thiadiazolidin-3-one-1,1-dioxide | |
| 363 | 5-{4-[2-[2-(3′-tert-Butyl-5′-methyl-biphenyl-4-yl)-(E)-vinyl]-4- | 686 (M + H)⁺ |
| | (2,4-dichloro-phenyl) imidazol-1-ylmethyl]-phenyl}-1,2,5- | |
| | thiadiazolidin-3-one-1,1-dioxide | |
| 364 | 5-{4-[2-{2-[4-(5-Chloro-thiophen-2-yl)-phenyl]-(E)-vinyl}-4- | 657 (M + H)⁺ |
| | (2,4-dichloro-phenyl)-imidazol-1-ylmethyl]-phenyl}-1,2,5- | |
| | thiadiazolidin-3-one-1,1-dioxide | |

### Example 365

{4-[2-[2-(4-Bromo-phenyl)-(E)-vinyl]-4-(2,4-dichloro-phenyl)-imidazol-1-ylmethyl]phenylamino}-acetic acid methyl ester (143 mg, 0.25 mmol) was coupled with 5-acetyl thiophene-2-phenylboronic acid (51 mg, 0.3 mmol) following general procedure B to give {4-[2-{2-[4-(5-Acetyl-thiophen-2-yl)-phenyl]-(E)-vinyl}-4-(2,4-dichloro-phenyl)-imidazol-1-ylmethyl]-phenylamino}-acetic acid methyl ester (113 mg, 73%).

{4-[2-{2-[4-(5-Acetyl-thiophen-2-yl)-phenyl]-(E)-vinyl}-4-(2,4-dichloro-phenyl) imidazol-1-ylmethyl]-phenylamino}-acetic acid methyl ester (62 mg, 0.1 mmol) was hydrolyzed according to general procedure F to give {4-[2-{2-[4-(5-acetyl-thiophen-2-yl)-phenyl]-(E)-vinyl}-4-(2,4-dichloro-phenyl)-imidazol-1-ylmethyl]-phenylamino}-acetic acid (51 mg, 80% yield).

LCMS: m/z 603 (M+H)⁺; ¹H NMR (DMSO-d₆, 400 MHz): δ 3.34 (s, 3H), 3.73 (d, 2H), 5.32 (s, 2H), 6.53 (d, 2H), 7.11 (d, 2H), 7.48-7.56 (m, 4H), 7.60 (d, 1H), 7.64 (d, 1H), 7.72 (s, 1H), 7.79 (d, 2H), 7.97-8.02 (m, 2H), 8.24 (d, 1H) ppm.

### Example 366

5-{4-[2-{2-[4-(5-Acetyl-thiophen-2-yl)-phenyl]-(E)-vinyl}-4-(2,4-dichloro-phenyl) imidazol-1-ylmethyl]-phenyl}-1,2,5-thiadiazolidin-3-one-1,1-dioxide (30 mg, 52%) was prepared according to general procedure Y from {4-[2-{2-[4-(5-acetyl-thiophen-2-yl)-phenyl]-(E)-vinyl}-4-(2,4-dichloro-phenyl)-imidazol-1-ylmethyl]-phenylamino}-acetic acid methyl ester (62 mg, 0.1 mmol).

LCMS: m/z 664 (M+H)⁺; ¹H NMR (DMSO-d₆, 400 MHz): δ 3.31 (s, 3H), 3.92 (s, 2H), 5.44 (s, 2H), 7.04 (d, 2H), 7.26 (d, 2H), 7.48-7.50 (m, 4H), 7.61 (d, 1H), 7.64 (d, 1H), 7.72 (d, 1H), 7.80 (d, 2H), 7.97 (d, 1H), 8.05 (d, 1H), 8.24 (d, 1H) ppm.

### Example 367

{4-[2-[2-(4-Bromo-phenyl)(E)vinyl]-4-(2,4-dichloro-phenyl)-imidazol-1-ylmethyl]phenylamino}-acetic acid methyl ester (143 mg, 0.25 mmol) was coupled with 2-fluoro-5 (trifluoromethyl)-phenylboronic acid (63 mg, 0.3 mmol) following general procedure B to give (4-{4-(2,4-Dichloro-phenyl)-2-[2-(2′-fluoro-5′-trifluoromethyl-biphenyl-4-yl)-(E)-vinyl]-imidazol-1-ylmethyl}-phenylamino)-acetic acid methyl ester (119 mg, 73%).

5-(4-{4-(2,4-Dichloro-phenyl)-2-[2-(2′-fluoro-g-trifluoromethyl-biphenyl-4-yl) vinyl]-imidazol-1-ylmethyl}-phenyl)-1,2,5-thiadiazolidin-3-one-1,1-dioxide (34 mg, 49% yield) was prepared according to general procedure Y from (4-{4-(2,4-Dichloro phenyl)-2-[2-(2′-fluoro-5′-trifluoromethyl-biphenyl-4-yl)-(E)-vinyl]-imidazol-1-ylmethyl}phenylamino)-acetic acid methyl ester (66 mg, 0.1 mmol).

LCMS: m/z 702 (M+H)⁺;

### Example 368

2-[2-(4-Bromo-phenyl)-(E)-vinyl]-4-(2,4-dichloro-phenyl)-1H-imidazole (400 mg, 1 mmol) was reacted with 4-nitobenzyl bromide (330 mg, 1.5 mmol) following general procedure E to give nitro compound (412 mg, 76%) and was coupled with 3-hydroxy-phenylboronic acid (115 mg, 0.8 mmol) following general procedure B to give 4′-{2-[1-(4-nitro-benzyl)-4-(2,4-dichloro-phenyl)-1H-imidazol-2-yl]-(E)-vinyl}-biphenyl-3-ol (319 mg, 75%).

4′-{2-[1-(4-Nitro-benzyl)-4-(2,4-dichloro-phenyl)-1H-imidazol-2-yl]-(E)-vinyl}-biphenyl-3-ol (300 mg, 0.56 mmol) was reacted with 1-bromo-3,3-dimethyl butane (99 mg, 0.6 mmol) following general procedure E to give O-alkylated nitro compound (265 mg, 75%) and was reduced to 4-(4-(2,4-dichloro-phenyl)-2-{2-[3′-(3,3-dimethyl-butoxy)-biphenyl-4-yl]-(E)-vinyl}-imidazol-1-ylmethyl)-phenylamine (195 mg, 77%) following general procedure K.

4-(4-(2,4-Dichloro-phenyl)-2-{2-[3′-(3,3-dimethyl-butoxy)-biphenyl-4-yl]-(E)-vinyl}-imidazol-1-ylmethyl)-phenylamine (150 mg, 0.25 mmol) was reacted with methyl bromo acetate (46 mg, 0.30 mmol) following general procedure L to give [4-(4-(2,4-dichloro-phenyl)-2-{2-[3′-(3,3-dimethyl-butoxy)-biphenyl-4-yl]-(E)-vinyl}-imidazol-1-ylmethyl)-phenylamino]-acetic acid methyl ester (129 mg, 77%).

5-[4-(4-(2,4-Dichloro-phenyl)-2-{2-[3′-(3,3-dimethyl-butoxy)-biphenyl-4-yl]-(E)-vinyl}imidazo-1-ylmethyl)-phenyl]-1,2,5-thiadiazolidin-3-one-1,1-dioxide (32 mg, 45%) was prepared according to general procedure Y from [4-(4-(2,4-dichloro-phenyl)-2-{2-[3′(3,3-dimethyl-butoxy)-biphenyl-4-yl]-(E)-vinyl}-imidazol-1-ylmethyl)-phenylamino]-acetic acid methyl ester (67 mg, 0.1 mmol).

LCMS: m/z 716 (M+H)⁺; ¹H NMR (DMSO-d₆, 400 MHz): δ 0.98 (s, 9H), 1.67 (m, 2H), 3.92 (s, 2H), 4.08 (m, 2H), 5.43 (s, 2H), 6.92 (d, 1H), 7.02 (d, 2H), 7.16 (d, 1H), 7.23-7.27 (m, 2H), 7.33-7.39 (m, 2H), 7.43 (s, 1H), 7.48-7.58 (m, 2H), 7.64 (d, 2H), 7.69 (d, 2H), 7.76 (d, 1H), 8.04 (s, 1H), 8.24 (d, 1H) ppm.

### Example 369

4′-{2-[1-(4-Nitro-benzyl)-4-(2,4-dichloro-phenyl)-1H-imidazol-2-yl]-(E)-vinyl}-biphenyl-3-ol (300 mg, 0.56 mmol) was reacted with 1-bromo-4,4,4-trifluorobutane (115 mg, 0.6 mmol) following general procedure E to give O-alkylated nitro compound (255 mg, 70%) and was reduced to 4-(4-(2,4-Dichloro-phenyl)-2-{2-[3′-(4,4,4-trifluoro-butoxy)-biphenyl-4-yl]-(E)-vinyl}-imidazol-1-ylmethyl)-phenylamine (185 mg, 76%) following general procedure K.

4-(4-(2,4-Dichloro-phenyl)-2-{2-[3′-(4,4,4-trifluoro-butoxy)-biphenyl-4-yl]-(E)-vinyl}-imidazol-1-ylmethyl)-phenylamine (156 mg, 0.25 mmol) was reacted with methyl bromo acetate (46 mg, 0.30 mmol) following general procedure L to give [4-(4-(2,4-dichloro-phenyl)-2-{2-[3′-(4,4,4-trifluoro-butoxy)-biphenyl-4-yl]-(E)-vinyl}-imidazol-1-ylmethyl)-phenylamino]-acetic acid methyl ester (129 mg, 77%).

5-[4-(4-(2,4-Dichloro-phenyl)-2-{2-[3′-(4,4,4-trifluoro-butoxy)-biphenyl-4-yl]-(E)-vinyl}-imidazol-1-ylmethyl)-phenyl]-1,2,5-thiadiazolidin-3-one-1,1-dioxide (35 mg, 47%) was prepared according to general procedure Y3 from [4-(4-(2,4-dichloro-phenyl)-2-{2-[3′-(4,4,4-trifluoro-butoxy)-biphenyl-4-yl]-(E)-vinyl}-imidazol-1-ylmethyl)-phenylamino]-acetic acid methyl ester (70 mg, 0.1 mmol).

LCMS: m/z 742 (M+H)⁺

### Example 370

5-[4-(4-(2,4-Dichloro-phenyl)-2-{2-[3′-fluoro-4′-(4,4,4-trifluoro-butoxy)-biphenyl-4-yl]-(E)-vinyl}-imidazol-1-ylmethyl)-phenyl]-1,2,5-thiadiazolidin-3-one-1,1-dioxide was prepared by analagous methods to those used to prepare Example 368.

LCMS: m/z 760 (M+H)⁺.

### Example 371

[4′-(2-{4-(2,4-Dichloro-phenyl)-1-[4-(1,1,4-trioxo-1-[1,2,5]thiadiazolidin-2-yl)benzyl]-1H-imidazol-2-yl}-(E)-vinyl)-4-fluoro-biphenyl-3-yl]-carbamic acid isopropyl ester prepared by analagous methods to those used to prepare Example 369.

LCMS: m/z 735 (M+H)⁺

### Example 372

4-[2-[2-(4-Bromo-phenyl)-(E)-vinyl]-4-(2,4-dichloro-phenyl)-imidazol-1-ylmethyl]phenylamine (0.165 mg, 0.33 mmol) was reacted with methyl 2-bromopropionate (59 mg, 0.35 mmol) following general procedure L to give 2-{4-[2-[2-(4-bromo-phenyl) vinyl]-4-(2,4-dichloro-phenyl)-imidazol-1-ylmethyl]-phenylamino}-propionic acid methyl ester (141 mg, 73%).

2-{4-[2-[2-(4-Bromo-phenyl)vinyl]-4-(2,4-dichloro-phenyl)-imidazol-1-ylmethyl]phenylamino}-propionic acid methylester (147 mg, 0.25 mmol) was coupled with 4-trifluoromethyl benzeneboronic acid (57 mg, 0.3 mmol) following general procedure B to give (2-(4-{4-(2,4-Dichloro-phenyl)-2-[2-(3′-trifluoromethyl-biphenyl-4-yl)-(E)-vinyl]-imidazol-1-ylmethyl}-phenylamino)-propionic acid methyl ester (126 mg, 77% yield).

5-(4-{4-(2,4-Dichloro-phenyl)-2-[2-(3′-trifluoromethyl-biphenyl-4-yl)-(E)-vinyl]imidazol-ylmethyl}-phenyl)-4-methyl-,2,5-thiadiazolidin-3-one-1,1-dioxide (32 mg, 46% yield) was prepared according to general procedure Y3 from (2-(4-{4-(2,4-dichloro-phenyl)-2-[2-(3′-trifluoromethyl-biphenyl-4-yl)-(E)-vinyl]-imidazol-1-ylmethyl}-phenyl-amino)-propionic acid methyl ester (65 mg, 0.1 mmol).

LCMS: m/z 698 (M+H)⁺; ¹H NMR (DMSO-d₆, 400 MHz): δ 1.22 (d, 3H), 4.27 (m, 1H), 5.46 (s, 2H), 7.12 (d, 2H), 7.26 (d, 2H), 7.46 (d, 2H), 7.50 (d, 2H), 7.57 (s, 1H), 7.61 (d, 1H), 7.63 (s, 1H), 7.72-7.83 (m, 4H), 8.02 (d, 2H), 8.27 (d, 1H) ppm.

### Example 373

Trans-2-fluoro-4-(trifluoromethyl)cinnamicacid (2.4 g, 10 mmol) was treated according to general procedure A using 2,4-dichlorophenacyl bromide to give 4-(2,4-dichloro-phenyl)-2-[2-(2-fluoro-4-trifluoromethyl-phenyl)-(E)-vinyl]-1H-imidazole (1.9 g, 46% yield). 4-(2,4-Dichloro-phenyl)-2-[2-(2-fluoro-4-trifluoromethyl-phenyl)-(E)-vinyl]-1H-imidazole (1.2 g, 3 mmol) was reacted with 4-nitobenzyl bromide (76 mg, 3.5 mmol) following general procedure E to give 4-(2,4-Dichloro-phenyl)-2-[2-(2-fluoro-4-trifluoro-methyl-phenyl)-(E)-vinyl]-1-(4-nitro-benzyl)-1H-imidazole (1.2 g, 75%).

LCMS: m/z 537 (M+H)⁺; ¹H NMR (DMSO-d₆, 400 MHz): δ 5.74 (s, 2H), 7.47-7.52 (m, 3H), 7.56 (s, 1H), 7.60-7.74 (m, 4H), 8.11-8.15 (m, 2H), 8.20 (s, 1H), 8.23-8.28 (m, 2H) ppm.

### Example 374

The compound of Example 373 (268 mg, 0.5 mmol) was reduced to 4-(2,4-Dichloro-phenyl)-2-[2-(2-fluoro-4-trifluoromethyl-phenyl)-(E)-vinyl]-1-(4-amino-benzyl)-1H-imidazole (192 mg, 78%) following general procedure K.

4-(2,4-Dichloro-phenyl)-2-[2-(2-fluoro-4-trifluoromethyl-phenyl)-(E)-vinyl]-1-(4-amino-benzyl)-1H-imidazole (169 mg, 0.33 mmol) was treated with methyl bromoacetate (54 mg, 0.35 mmol) according to general procedure E to give (4-{4-(2,4-dichloro-phenyl) 2-[2-(2-fluoro-4-trifluoromethyl-phenyl)-(E)-vinyl]-imidazol-1-ylmethyl}-phenylamino)-acetic acid methyl ester (159 mg, 82%).

5-(4-{4-(2,4-Dichloro-phenyl)-2-[2-(2-fluoro-4-trifluoromethyl-phenyl)-(E)-vinyl]-imidazol-1-ylmethyl}-phenyl)-1,2,5-thiadiazolidin-3-one-1,1-dioxide (32 mg, 50% yield) was prepared according to general procedure Y3 from (4-{4-(2,4-dichloro-phenyl)-2-[2-(2-fluoro-4-trifluoromethyl-phenyl)-(E)-vinyl]-imidazol-1-ylmethyl}-phenylamino)-acetic acid methyl ester (58 mg, 0.1 mmol).

LCMS: m/z 626 (M+H)⁺; ¹H NMR (DMSO-d₆, 400 MHz): δ 3.93 (s, 2H), 5.43 (s, 2H), 6.97 (d, 2H), 7.04 (d, 2H), 7.16 (s, 1H), 7.21-7.25 (m, 2H), 7.47-7.50 (m, 2H), 7.56 (s, 1H), 7.62 (d, 2H), 7.97 (s, 1H), 8.27 (d, 1H) ppm.

### Example 375

The compound of Example 374 (63 mg, 0.1 mmol) was treated with iodo methane (16 mg, 0.11 mmol) according to general procedure L to give 5-(4-{4-(2,4-Dichloro-phenyl)-2-[2-(2-fluoro-4-trifluoromethyl-phenyl)-(E)-vinyl]-imidazol-1-ylmethyl}-phenyl)-2-methyl-2,5-thiadiazolidin-3-on-1,1-dioxide (55 mg, 85%).

LCMS: m/z 640 (M+H)⁺

### Example 376

Trans-4-hydroxy cinnamic acid (1.6 g, 1 mmol) was treated according to general procedure A using 2,4-dichlorophenacyl bromide to give 4-{2-[4-(2,4-dichloro-phenyl)-1H-imidazol-2-yl]-(E)-vinyl}-phenol (1.8 g, 56% yield).

4-{2-[4-(2,4-Dichloro-phenyl)-1H-imidazol-2-yl]-(E)-vinyl}-phenol (1 g, 3 mmol) was reacted with 4-nitobenzyl bromide (76 mg, 3.5 mmol) following general procedure E to give 4-{2-[1-(4-nitro-benzyl)-4-(2,4-dichloro-phenyl)-1H-imidazol-2-yl]-(E)-vinyl}-phenol (900 mg, 64%).

4-{2-[1-(4-nitro-benzyl)-4-(2,4-dichloro-phenyl)-1H-imidazol-2-yl]-(E)-vinyl}-phenol (467 mg, 1 mmol) was treated with 1-bromo-4,4,4-trifluorobutane (210 mg, 1.1 mmol) following general procedure E and was reduced to 4-(4-(2,4-dichloro-phenyl)-2-{2-[4-(4,4,4-trifluoro-butoxy)-phenyl]-(E)-vinyl}-imidazol-1-ylmethyl)-phenylamine (350 mg, 64% yield) following general procedure K.

4-(4-(2,4-Dichloro-phenyl)-2-{2-[4-(4,4,4-trifluoro-butoxy)-phenyl]-(E)-vinyl}-imidazol-1-ylmethyl)-phenylamine (274 mg, 0.5 mmol) was reacted with methyl bromo acetate (85 mg, 0.55 mmol) and was treated with chlorosulfonylisocynate and tert-butanol according to general procedure Y3 to give {[4-(4-(2,4-Dichloro-phenyl)-2-{2-[4-(4,4,4-trifluoro-butoxy)-phenyl]-(E)-vinyl}-imidazol-1-ylmethyl)-phenyl]-N-Boc-sulfonyl-amino}-acetic acid methyl ester (181 mg, 45%).

{[4-(4-(2,4-Dichloro-phenyl)-2-{2-[4-(4,4,4-trifluoro-butoxy)-phenyl]-(E)-vinyl}-imidazol-1-ylmethyl)-phenyl]-N-Boc-sulfonyl-amino}-acetic acid methyl ester (150 mg, 0.18 mmol) was hydrolyzed following general procedure F to give {[4-(4-(2,4-dichloro-phenyl)-2-{2-[4-(4,4,4-trifluoro-butoxy)-phenyl]-(E)-vinyl}-imidazol-1-ylmethyl)-phenyl]-N-Boc-sulfonyl-amino}-acetic acid (120 mg, 81%).

LCMS: m/z 784 (M+H)⁺.

### Example 377

The compound of Example 376 (79 mg, 0.1 mmol) was treated with 2N—HCl following general procedure O to give {[4-(4-(2,4-dichloro-phenyl)-2-{2-[4-(4,4,4-trifluoro-butoxy)-phenyl]-(E)-vinyl}-imidazol-1-ylmethyl)-phenyl]-N-sulfonyl-amino}-acetic acid (52 mg, 77% yield). LCMS: m/z 684 (M+H)⁺

### Example 378

5-[4-(4-(2,4-Dichloro-phenyl)-2-{2-[4-(4,4,4-trifluoro-butoxy)-phenyl]-(E)-vinyl}imidazol-1-ylmethyl)-phenyl]-1,2,5-thiadiazolidin-3-one-1,1-dioxide (35 mg, 53% Yield) was prepared according to general procedure Y from [4-(4-(2,4-Dichloro-phenyl)-2-{2-[4-(4,4,4-trifluoro-butoxy)-phenyl]-(E)-vinyl}-imidazol-1-ylmethyl)-phenylamino]-acetic acid methyl ester (62 mg, 0.1 mmol).

LCMS: m/z 666 (M+H)⁺; ¹H NMR (DMSO-d₆, 400 MHz): δ 1.90 (m, 2H), 2.40 (m, 2H), 3.93 (s, 2H), 4.06 (m, 2H), 5.39 (s, 2H), 6.96 (d, 2H), 7.03 (d, 2H), 7.18 (s, 1H), 7.22-0.25 (m, 2H), 7.44-7.49 (m, 4H), 7.62 (d, 2H), 7.99 (s, 1H), 8.22 (d, 1H) ppm.

### Example 379

4-Trifluoromethylhydrocinnamic acid (2.2 g, 10 mmol) was treated according to general procedure A using 2,4-dichlorophenacyl bromide to give 4-(2,4-dichloro-phenyl)-2-[2-(4-trifluoromethyl-phenyl)-ethyl]-1H-imidazole (1.8 g, 47% yield).

4-(2,4-Dichloro-phenyl)-2-[2-(4-trifluoromethyl-phenyl)-ethyl]-1H-imidazole (193 mg, 0.5 mmol) was reacted with 4-nitobenzyl bromide (120 mg, 3.5 mmol) following general procedure E to give 4-(2,4-dichloro-phenyl)-1-(4-nitro-benzyl)-2-[2-(4-trifluoromethyl-phenyl)-ethyl]-1H-imidazole (191 mg, 72%).

LCMS: m/z 521 (M+H)⁺

### Example 380

4-{2-[1-(4-nitro-benzyl)-4-(2,4-dichloro-phenyl)-1H-imidazol-2-yl]-(E)-vinyl}-phenol (467 mg, 1 mmol) was treated with 4-tert-butyl benzene boronic acid (196 mg, 1.1 mmol) following general procedure w to give 2-{2-[4-(4-tert-Butyl-phenoxy)-phenyl]-(E)-vinyl}-4-(2,4-dichloro-phenyl)-1-(4-nitro-benzyl)-1H-imidazole (385 mg, 64% yield).

LCMS: m/z 599 (M+H)⁺

### Example 381

2-{2-[4-(4-tert-Butyl-phenoxy)-phenyl]-(E)-vinyl}-4-(2,4-dichloro-phenyl)-1-(4-nitro-benzyl)-1H-imidazole (300 mg, 0.5 mmol) was reduced to 4-amino compound and was N-alkylated with methyl bromoacetate (85 mg, 0.55 mmol) to give {4-[2-{2-[4-(4-tert-butyl-phenoxy)-phenyl]-(E)-vinyl}-4-(2,4-dichloro-phenyl)-imidazol-1-ylmethyl]-phenylamino}-acetic acid methyl ester (218 mg, 68%).

5-[4-(4-(2,4-Dichloro-phenyl)-2-{2-[4-(4,4,4-trifluoro-butoxy)-phenyl]-(E)-vinyl}-imidazol-1-ylmethyl)-phenyl]-1,2,5-thiadiazolidin-3-one-1,1-dioxide (34 mg, 49%) was prepared from {4-[2-{2-[4-(4-tert-butyl-phenoxy)-phenyl]-(E)-vinyl}-4-(2,4-dichloro-phenyl)-imidazol-1-ylmethyl]-phenylamino}-acetic acid methyl ester (65 mg, 0.1 mmol) following general procedure Y3.

LCMS: m/z 688 (M+H)⁺; ¹H NMR (DMSO-d₆, 400 MHz): δ 1.28 (s, 9H), 3.93 (s, 2H), 5.40 (s, 2H), 6.97 (d, 2H), 7.03 (d, 1H), 7.24 (d, 1H), 7.40 (s, 1H), 7.41-7.48 (m, 2H), 7.49 (s, 1H), 7.52-7.62 (m, 4H), 7.68 (d, 2H), 7.71 (d, 2H), 8.02 (s, 1H), 8.23 (d, 1H) ppm.

### Example 382

4-{2-[1-(4-nitro-benzyl)-4-(2,4-dichloro-phenyl)-1H-imidazol-2-yl]-(E)-vinyl}-phenol (467 mg, 1 mmol) was treated with 4-(trifluoromethyl)benzene boronic acid (210 mg, 1.1 mmol) following general procedure w to give 2-{2-[4-(4-trifluoromethyl-phenoxy)-phenyl]-(E)-vinyl}-4-(2,4-dichloro-phenyl)-1-(4-nitro-benzyl)-1H-imidazole (389 mg, 64%).

2-{2-[4-(4-trifluoromethyl-phenoxy)-phenyl]-(E)-vinyl}-4-(2,4-dichloro-phenyl)-1-(4-nitro-benzyl)-1H-imidazole (306 mg, 0.5 mmol) was reduced to 4-amino compound and was N-alkylated with methyl bromoacetate (85 mg, 0.55 mmol) to give {4-[2-{2-[4-(4-trifluoromethyl-phenoxy)-phenyl]-(E)-vinyl}-4-(2,4-dichloro-phenyl)-imidazol-1-ylmethyl]-phenylamino}-acetic acid methyl ester (226 mg, 68% yield).

{4-[2-{2-[4-(4-trifluoromethyl-phenoxy)-phenyl]-(E)-vinyl}-4-(2,4-dichloro-phenyl)-imidazol-1-ylmethyl]-phenylamino}-acetic acid methyl ester (65 mg, 0.1 mmol) was hydrolyzed to {4-[2-{2-[4-(4-trifluoromethyl-phenoxy)-phenyl]-(E)-vinyl}-4-(2,4-dichloro-phenyl)-imidazol-1-ylmethyl]-phenylamino}-acetic acid (56 mg, 88% yield) following general procedure F.

LCMS: m/z 639 (M+H)⁺

### Example 383

5-[4-(4-(2,4-Dichloro-phenyl)-2-{2-[4-(4-trifluoromethyl-phenoxy)-phenyl]-(E)-vinyl}-imidazol-1-ylmethyl)-phenyl]-1,2,5]thiadiazolidin-3-one-1,1-dioxide (36 mg, 50% yield) was prepared from {4-[2-{2-[4-(4-trifluoromethyl-phenoxy)-phenyl]-(E)-vinyl}-4-(2,4 dichloro-phenyl)imidazol-1-ylmethyl]-phenylamino}-acetic acid methyl ester (65 mg, 0.1 mmol) following general procedure Y3.

LCMS: m/z 688 (M+H)⁺; ¹H NMR (DMSO-d₆, 400 MHz): δ 3.92 (s, 2H), 5.43 (s, 2H), 6.99 (d, 2H), 7.07 (d, 1H), 7.27 (d, 1H), 7.43 (s, 1H), 7.47-7.53 (m, 2H), 7.54 (s, 1H), 7.55-7.66 (m, 4H), 7.69 (d, 2H), 7.73 (d, 2H), 8.09 (s, 1H), 8.27 (d, 1H) ppm.

### Example 384

4-{4-(2,4-Dichloro-phenyl)-2-[2-(3′-trifluoromethyl-biphenyl-4-yl)-(E)-vinyl]-imidazol-1-ylmethyl}-benzoic acid (60 mg, 0.1 mmol) was reacted with ammonia in 2.0 M solution methanol following general procedure G to 4-{4-(2,4-dichloro-phenyl)-2-[2-(3′-trifluoromethyl-biphenyl-4-yl)-(E)-vinyl]-imidazol-1-ylmethyl}-benzamide (49 mg, 83%).

LCMS: m/z 593 (M+H)⁺; ¹H NMR (DMSO-d₆, 400 MHz): δ 5.62 (s, 2H), 7.33-7.39 (m, 2H), 7.43 (s, 1H), 7.50 (d, 1H), 7.52 (d, 1H), 7.57 (s, 1H), 7.61 (s, 1H), 7.65 (d, 2H), 7.69 (d, 2H), 7.71-7.82 (m, 4H), 7.84 (d, 1H), 8.01 (d, 2H), 8.11 (s, 1H), 8.27 (d, 1H) ppm.

### Example 385

2-[2-(4-Bromo-phenyl)-(E)-vinyl]-4-(2,4-dichloro-phenyl)-1H-imidazole (4.0 g, 10 mmol) was reacted with methyl 4-nitro benzyl bromide (3.2 g, 15 mmol) following general procedure E to give 2-[2-(4-Bromo-phenyl)-(E)-vinyl]-4-(2,4-dichloro-phenyl)-1-(4-nitro-benzyl)-1H-imidazole (4.1 g, 77%).

2-[2-(4-Bromo-phenyl)-(E)-vinyl]-4-(2,4-dichloro-phenyl)-1-(4-nitro-benzyl)-1H-imidazole (2.6 g, 5 mmol) was reduced according to general procedure K to give amino compound (1.8 g, 75%) and was treated with methanesulfonyl chloride (450 mg, 3.9 mmol) to give N-{4-[2-[2-(4-bromo-phenyl)-(E)-vinyl]-4-(2,4-dichloro-phenyl)-imidazol-1-ylmethyl]-phenyl}-methanesulfonamide (1.2 g, 60% Yield).

N-{4-[2-[2-(4-Bromo-phenyl)-(E)-vinyl]-4-(2,4-dichloro-phenyl)-imidazol-1-ylmethyl]-phenyl}-methanesulfonamide (289 mg, 0.5 mmol) was coupled with 3-methoxy carbonyl phenyl boronic acid (99 mg, 0.55 mmol) following general procedure B to give 4′-{2-[4-(2,4-dichloro-phenyl)-1-(4-methanesulfonylamino-benzyl)-1H-imidazol-2-yl]-(E)-vinyl}-biphenyl-3-carboxylic acid methyl ester (217 mg, 68%).

4′-{2-[4-(2,4-Dichloro-phenyl)-1-(4-methanesulfonylamino-benzyl)-1H-imidazol-2-yl]-(E)-vinyl}-biphenyl-3-carboxylic acid methyl ester (64 mg, 01 mmol) was hydrolyzed following general procedure F to give 4′-{2-[4-(2,4-dichlorophenyl)-1-(4-methanesulfonyl-amino-benzyl)-1H-imidazol-2-yl]-(E)-vinyl}-biphenyl-3-carboxylic acid (51 mg, 82%)

LCMS: m/z 619 (M+H)⁺; ¹H NMR (DMSO-d₆, 400 MHz): δ 2.95 (s, 3H), 5.49 (s, 2H), 6.55 (d, 1H), 6.75 (d, 1H), 7.16-7.21 (m, 2H), 7.23 (d, 2H), 7.29-7.31 (m, 2H), 7.41-7.51 (m, 2H), 7.55 (d, 1H), 7.62-7.74 (m, 2H), 7.80 (d, 1H), 7.94 (d, 1H), 8.06 (s, 1H), 8.14 (d, 1H), 8.28 (d, 1H), 9.80 (s, 1H) ppm.

### Example 386

2-[2-(4-Bromo-phenyl)-(E)-vinyl]-4-(2,4-dichloro-phenyl)-1H-imidazole (4.0 g, 10 mmol) was reacted with methyl 4-(trifluoromethoxy)-benzyl bromide (3.8 g, 15 mmol) following general procedure E to give 2-[2-(4-bromo-phenyl)-(E)-vinyl]-4-(2,4-dichloro-phenyl)-1-(4-trifluoromethoxy-benzyl)-1H-imidazole (3.9 g, 68%).

2-[2-(4-Bromo-phenyl)-(E)-vinyl]-4-(2,4-dichloro-phenyl)-1-(4-trifluoromethoxy-benzyl)-1H-imidazole (285 mg, 0.5 mmol) was coupled with 3-methoxy carbonyl phenyl boronic acid (99 mg, 0.55 mmol) following general procedure B to give 4′-{2-[4-(2,4-dichloro-phenyl)-1-(4-trifluoromethoxy-benzyl)-1H-imidazol-2-yl]-(E)-vinyl}-biphenyl-3-carboxylic acid methyl ester (209 mg, 67%).

4′-{2-[4-(2,4-Dichloro-phenyl)-1-(4-trifluoromethoxy-benzyl)-1H-imidazol-2-yl]-(E)-vinyl}-biphenyl-3-carboxylic acid methyl ester (63 mg, 01 mmol) was hydrolyzed following general procedure F to give 4′-{2-[4-(2,4-Dichloro-phenyl)-1-(4-trifluoro-methoxy-benzyl)-1H-imidazol-2-yl]-(E)-vinyl}-biphenyl-3-carboxylic acid (50 mg, 82%)

LCMS: m/z 610 (M+H)⁺; ¹H NMR (DMSO-d₆, 400 MHz): δ 5.60 (s, 2H), 6.98 (d, 1H), 7.28-7.59 (m, 6H), 7.61 (d, 1H), 7.62-7.72 (m, 3H), 7.74 (d, 2H), 7.93 (d, 2H), 7.97 (s, 1H), 8.11 (s, 1H), 8.27 (d, 1H) ppm.

### Example 387

4-(4-(2,4-Dichloro-phenyl)-2-{2-[4-(3-trifluoromethyl-benzenesulfonylamino)-phenyl]-(E)-vinyl}-imidazol-1-ylmethyl)-benzoic acid (24 mg, 0.036 mmol) was treated with sodium hydride and methyl iodide according to general procedure P to provide 4-[4-(2,4-dichloro-phenyl)-2-(2-{4-[methyl-(3-trifluoromethyl-benzenesulfonyl)-amino]-phenyl}-(E)-vinyl)-imidazol-1-ylmethyl]-benzoic acid methyl ester (19 mg, 76% yield).

LCMS: m/z 700 (M+H)⁺. ¹H NMR (CDCl₃, 400 MHz): δ 3.18 (s, 3H), 3.92 (s, 3H), 5.35 (s, 2H), 6.77 (d, 1H), 7.05 (m, 2H), 7.24 (d, 2H), 7.34 (dd, 1H), 7.38 (d, 2H), 7.43 (d, 1H), 7.58-7.73 (m, 4H), 7.79 (s, 1H), 7.85 (d, 1H), 8.05 (m, 2H), 8.26 (d, 1H) ppm.

### Example 388

4-[4-(2,4-dichloro-phenyl)-2-(2-{4-[methyl-(4-trifluoromethyl-benzenesulfonyl)-amino]-phenyl}-(E)-vinyl]-imidazol-1-ylmethyl]-benzoic acid methyl ester prepared by analagous methods to those used to prepare Example 387. (16 mg, 64% yield).

LCMS: m/z 700 (M+H)⁺.

### Example 389

The compound of Example 387 (19 mg, 0.027 mmol) was hydrolyzed according to general procedure F to provide 4-[4-(2,4-dichloro-phenyl)-2-(2-{4-[methyl-(3-trifluoromethyl-benzenesulfonyl)-amino]-phenyl}-(E)-vinyl)-imidazol-1-ylmethyl]-benzoic acid (12 mg, 64% yield). LCMS: m/z 686 (M+H)⁺.

### Example 390

The compound of Example 388 (16 mg, 0.023 mmol) was hydrolyzed according to general procedure F to provide 4-[4-(2,4-dichloro-phenyl)-2-(2-{4-[methyl-(4-trifluoromethyl-benzenesulfonyl)-amino]-phenyl}-(E)-vinyl)-imidazol-1-ylmethyl]-benzoic acid (12 mg, 76% yield). LCMS: m/z 686 (M+H)⁺.

### Example 391

3-Boc-amino-phenylacetic acid and 2,4-dichlorophenacyl bromide were reacted according to general procedure A. The obtained imidazole was alkylated with methyl 4-bromomethyl benzoate using general procedure E, then deprotected using general procedure O to provide 4-[2-(3-amino-benzyl)-4-(2,4-dichloro-phenyl)-imidazol-1-ylmethyl]-benzoic acid methyl ester (364 mg, 0.8 mmol). Treatment of the 3-amino compound (49 mg, 0.1 mmol) with 4-n-butylbenzenesulfonyl chloride according to general procedure L provided 4-[2-[3-(4-butyl-benzenesulfonylamino)-benzyl]-4-(2,4-dichloro-phenyl)-imidazol-1-ylmethyl]-benzoic acid methyl ester (57 mg, 82% yield)

LCMS: m/z 662 (M+H)⁺. ¹H NMR (CDCl₃, 400 MHz): δ 0.89 (t, 3H), 1.30 (m, 2H), 1.50 (m, 2H), 2.55 (t, 2H), 3.92 (s, 3H), 3.99 (s, 2H), 4.88 (s, 2H), 6.84 (d, 1H), 6.89 (s, 1H), 6.94-7.00 (m, 3H), 7.08-7.12 (m, 3H), 7.14 (dd, 1H), 7.41 (d, 1H), 7.54 (s, 1H), 7.61 (m, 2H), 7.87 (s, 1H), 7.93 (m, 2H), 8.05 (d, 1H) ppm.

### Example 392

The compound of Example 391 (50 mg, 0.076 mmol) was hydrolyzed according to general procedure F to give 4-[2-[3-(4-Butyl-benzenesulfonylamino)-benzyl]-4-(2,4-dichloro-phenyl)-imidazol-1-ylmethyl]-benzoic acid (42 mg, 86% yield).

LCMS: m/z 648 (M+H)⁺.

### Example 393

3-Amino-4-{4-(2,4-dichloro-phenyl)-2-[2-(4′-trifluoromethyl-biphenyl-4-yl)-(E)-vinyl]-imidazol-1-ylmethyl}-benzoic acid methyl ester (32 mg, 0.05 mmol) was treated with methanesulfonyl chloride according to general procedure L to provide 4-{4-(2,4-dichloro-phenyl)-2-[2-(4′-trifluoromethyl-biphenyl-4-yl)-(E)-vinyl]-imidazol-1-ylmethyl}-3-methanesulfonylamino-benzoic acid methyl ester (10 mg, 28% yield).

LCMS: m/z 700 (M+H)⁺. ¹H NMR (CD₃OD, 400 MHz): δ 3.14 (s, 3H), 3.92 (s, 3H), 5.72 (s, 2H), 6.95 (d, 1H), 7.03 (d, 1H), 7.38 (dd, 1H), 7.48 (d, 1H), 7.63 (m, 4H), 7.68 (d, 1H), 7.72 (m, 4H), 7.76 (s, 1H), 7.94 (dd, 1H) 7.98 (d, 1H), 8.09 (d, 1H) ppm.

### Example 394

The compound of Example 393 (8 mg, 0.01 mmol) was hydrolyzed according to general procedure F to provide 4-{4-(2,4-dichloro-phenyl)-2-[2-(4′-trifluoromethyl-biphenyl-4-yl)-(E)-vinyl]-imidazol-1-ylmethyl}-3-methanesulfonylamino-benzoic acid (6 mg, 76% yield).

LCMS: m/z 686 (M+H)⁺.

### Example 395

3-[2-[2-(4-Bromo-phenyl)-(E)-vinyl]-4-(2,4-dichloro-phenyl)-imidazol-1-ylmethyl]-benzoic acid methyl ester was coupled with 4-(Boc-amino)-3-methoxyphenyl boronic acid according to general procedure B to provide 3-[2-[2-(4′-tert-butoxycarbonylamino-3′-methoxy-biphenyl-4-yl)-(E)-vinyl]-4-(2,4-dichloro-phenyl)-imidazol-1-ylmethyl]-benzoic acid methyl ester. 15 mg (0.02 mmol) of the ester was hydrolyzed according to general procedure F to provide 3-[2-[2-(4′-tert-butoxycarbonylamino-3′-methoxy-biphenyl-4-yl)-(E)-vinyl]-4-(2,4-dichloro-phenyl)-imidazol-1-ylmethyl]-benzoic acid (12 mg, 82% yield).

LCMS: m/z 670 (M+H)⁺. ¹H NMR (CD₃OD, 400 MHz): δ 1.54 (s, 9H), 3.95 (s, 3H), 5.53 (s, 2H), 7.15 (d, 1H), 7.18-7.22 (m, 2H), 7.40 (dd, 1H), 7.47-7.51 (m, 2H), 7.54 (d, 1H), 7.57-7.64 (m, 4H), 7.80 (s, 1H), 7.84 (s, 1H), 7.91 (d, 1H) 7.97 (m, 1H), 7.99-8.03 (m, 2H) ppm.

### Example 396

The compound of Example 395 (45 mg, 0.066 mmol) was deprotected according to general procedure O, then treated with isopropyl chloroformate using general procedure L to provide 3-{4-(2,4-dichloro-phenyl)-2-[2-(4′-isopropoxycarbonylamino-3′-methoxy-biphenyl-4-yl)-(E)-vinyl]-imidazol-1-ylmethyl}-benzoic acid methyl ester (27 mg, 61% yield).

LCMS: m/z 670 (M+H)⁺. ¹H NMR (CD₃OD, 400 MHz): δ 1.33 (d, 6H), 3.89 (s, 3H), 3.95 (s, 3H), 4.99 (sept, 1H), 5.44 (s, 2H), 7.04 (d, 1H), 7.15 (d, 1H), 7.19 (dd, 1H), 7.36 (dd, 1H), 7.46-7.49 (m, 2H), 7.54-7.63 (m, 5H), 7.74 (s, 1H), 7.95-8.05 (m, 5H) ppm.

### Example 397

3-{4-(2,4-Dichloro-phenyl)-2-[2-(4′-isopropoxycarbonylamino-3′-methoxy-biphenyl-4-yl)-(E)-vinyl]-imidazol-1-ylmethyl}-benzoic acid methyl ester (22 mg, 0.033 mmol) was hydrolyzed according to general procedure F to provide 3-{4-(2,4-dichloro-phenyl)-2-[2-(4′-isopropoxycarbonylamino-3′-methoxy-biphenyl-4-yl)-(E)-vinyl]-imidazol-1-ylmethyl}-benzoic acid (9 mg, 42% yield).

LCMS: m/z 656 (M+H)⁺. ¹H NMR (CD₃OD, 400 MHz): δ 1.34 (d, 6H), 3.96 (s, 3H), 4.98 (sept, 1H), 5.50 (s, 2H), 7.11 (d, 1H), 7.18-7.23 (m, 2H), 7.38 (dd, 1H), 7.47-7.51 (m, 3H), 7.57-7.64 (m, 5H), 7.77 (s, 1H), 7.96-8.04 (m, 4H) ppm.

### Example 398

4-Bromomethyl-2,3-difluoro-benzoic acid methyl ester (prepared by esterification and benzylic bromination of 2,3-difluoro-4-methyl benzoic acid) was employed to alkylate 2-[2-(4-bromo-phenyl)-(E)-vinyl]-4-(2,4-dichloro-phenyl)-1H-imidazole using general procedure E. The product, 4-[2-[2-(4-bromo-phenyl)-(E)-vinyl]-4-(2,4-dichloro-phenyl)-imidazol-1-ylmethyl]-2,3-difluoro-benzoic acid methyl ester was coupled with 4-(Boc-amino)-3-methoxyphenyl boronic acid according to general procedure B to provide 4-[2-[2-(4′-tert-butoxycarbonylamino-3′-methoxy-biphenyl-4-yl)-(E)-vinyl]-4-(2,4-dichloro-phenyl)-imidazol-1-ylmethyl]-2,3-difluoro-benzoic acid methyl ester (71 mg, 0.1 mmol). The Boc group was removed according to general procedure O, and the crude product was treated with isopropyl chloroformate using general procedure L to provide 4-{4-(2,4-dichloro-phenyl)-2-[2-(4′-isopropoxycarbonylamino-3′-methoxy-biphenyl-4-yl)-(E)-vinyl]-imidazol-1-ylmethyl}-2,3-difluoro-benzoic acid methyl ester (36 mg, 52% yield).

LCMS: m/z 706 (M+H)⁺. ¹H NMR (CD₃OD, 400 MHz): δ 1.35 (d, 6H), 3.93 (s, 3H), 3.97 (s, 3H), 5.00 (sept, 1H), 5.52 (s, 2H), 6.92 (m, 1H), 7.03 (d, 1H), 7.15 (d, 1H), 7.21 (dd, 1H), 7.36 (dd, 1H), 7.47 (d, 1H), 7.61 (s, 4H), 7.65 (d, 1H), 7.70 (m, 1H), 7.76 (s, 1H), 7.99-8.07 (m, 2H) ppm.

### Example 399

The compound of Example 398 (33 mg, 0.047 mmol) was hydrolyzed according to general procedure F to provide 4-{4-(2,4-dichloro-phenyl)-2-[2-(4′-isopropoxycarbonylamino-3′-methoxy-biphenyl-4-yl)-(E)-vinyl]-imidazol-1-ylmethyl}-2,3-difluoro-benzoic acid (27 mg, 83% yield).

LCMS: m/z 692 (M+H)⁺.

### Example 400

4-Bromomethyl-3-trifluoromethyl-benzoic acid methyl ester (prepared by esterification and benzylic bromination of 3-trifluoromethyl-4-methyl-benzoic acid) was employed to alkylate2-[2-(4-bromo-phenyl)-(E)-vinyl]-4-(2,4-dichloro-phenyl)-1H-imidazole using general procedure E. The product, 4-[2-[2-(4-bromo-phenyl)-(E)-vinyl]-4-(2,4-dichloro-phenyl)-imidazol-1-ylmethyl]-3-trifluoromethyl-benzoic acid methyl ester was coupled with 4-(Boc-amino)-3-methoxyphenyl boronic acid according to general procedure B to provide 4-[2-[2-(4′-tert-butoxycarbonylamino-3′-methoxy-biphenyl-4-yl)-(E)-vinyl]-4-(2,4-dichloro-phenyl)-imidazol-1-ylmethyl]-3-trifluoromethyl-benzoic acid methyl ester (68 mg, 0.09 mmol). The Boc group was removed according to general procedure O, and the crude product was treated with isopropyl chloroformate using general procedure L to provide 4-{4-(2,4-dichloro-phenyl)-2-[2-(4′-isopropoxycarbonyl-amino-3′-methoxy-biphenyl-4-yl)-(E)-vinyl]-imidazol-1-ylmethyl}-3-trifluoromethyl-benzoic acid methyl ester (38 mg, 57% yield).

LCMS: m/z 738 (M+H)⁺. ¹H NMR (CD₃OD, 400 MHz): δ 1.34 (d, 6H), 3.96 (m, 6H), 5.00 (sept, 1H), 5.63 (s, 2H), 6.79 (d, 1H), 7.00 (d, 1H), 7.11 (d, 1H), 7.19 (dd, 1H), 7.38 (dd, 1H), 7.47-7.52 (m, 3H), 7.59 (d, 1H), 7.76 (s, 1H), 8.04 (m, 1H), 8.12 (d, 1H), 8.17 (d, 1H), 8.43 (s, 1H) ppm.

### Example 401

The compound of Example 400 (35 mg, 0.047 mmol) was hydrolyzed according to general procedure F to provide 4-{4-(2,4-dichloro-phenyl)-2-[2-(4′-isopropoxycarbonylamino-3′-methoxy-biphenyl-4-yl)-(E)-vinyl]-imidazol-1-ylmethyl}-3-trifluoromethyl-benzoic acid (19 mg, 55% yield). LCMS: m/z 724 (M+H)⁺.

### Example 402

4-Bromomethyl-2-fluoro-benzoic acid methyl ester (prepared by esterification and benzylic bromination of 2-fluoro-4-methyl benzoic acid) was employed to alkylate2-[2-(4-bromo-phenyl)-(E)-vinyl]-4-(2,4-dichloro-phenyl)-1H-imidazole using general procedure E. The product, 4-[2-[2-(4-bromo-phenyl)-(E)-vinyl]-4-(2,4-dichloro-phenyl)-imidazol-1-ylmethyl]-3-trifluoromethyl-benzoic acid methyl ester (350 mg, 0.62 mmol) was coupled with 3-methanesulfonylphenyl boronic acid according to general procedure B to provide 4-{4-(2,4-dichloro-phenyl)-2-[2-(3′-methanesulfonyl-biphenyl-4-yl)-(E)-vinyl]-imidazol-1-ylmethyl}-2-fluoro-benzoic acid methyl ester (88 mg, 22% yield).

LCMS: m/z 635 (M+H)⁺. ¹H NMR (DMSO-d₆, 400 MHz): δ 3.33 (s, 3H), 3.87 (s, 3H), 5.33 (s, 2H), 6.87 (d, 1H), 7.38-7.47 (m, 3H), 7.75-7.82 (m, 3H), 7.84-7.89 (m, 3H), 7.91-7.97 (m, 5H), 8.11 (d, 1H), 8.23 (m, 1H) ppm.

### Example 403

The compound of Example 402 (85 mg, 0.13 mmol)) was hydrolyzed according to general procedure F to provide 4-{4-(2,4-dichloro-phenyl)-2-[2-(3′-methanesulfonyl-biphenyl-4-yl)-(E)-vinyl]-imidazol-1-ylmethyl}-2-fluoro-benzoic acid (60 mg, 72% yield).

LCMS: m/z 621 (M+H)⁺.

### Example 404

(4-{4-(2,4-Dichloro-phenyl)-2-[2-(3′-methanesulfonyl-biphenyl-4-yl)-(E)-vinyl]-imidazol-1-ylmethyl}-phenylamino)-acetic acid methyl ester (100 mg, 0.16 mmol)) was cyclized according to general procedure Y to provide 5-(4-{4-(2,4-dichloro-phenyl)-2-[2-(3′-methanesulfonyl-biphenyl-4-yl)-(E)-vinyl]-imidazol-1-ylmethyl}-phenyl)-1,2,5-thiadiazolidin-3-one-1,1-dioxide (16 mg, 15% yield).

LCMS: m/z 693 (M+H)⁺. ¹H NMR (DMSO-d₆, 400 MHz): δ 3.33 (s, 3H), 3.94 (s, 2H), 5.46 (s, 2H), 7.06 (d, 2H), 7.28 (d, 2H), 7.45-7.52 (m, 2H), 7.59 (d, 1H), 7.65 (d, 1H), 7.76 (t, 1H), 7.81-7.88 (m, 4H), 7.92 (m, 1H), 8.06 (s, 1H), 8.10 (m, 1H), 8.21 (m, 1H), 8.27 (d, 1H) ppm.

### Example 405

(4-{2-[4-(2,4-Dichloro-phenyl)-1-ethyl-1H-imidazol-2-yl]-(E)-vinyl}-phenylamino)-acetic acid methyl ester (267 mg, 0.62 mmol)) was cyclized according to general procedure Y to provide 5-(4-{2-[4-(2,4-dichloro-phenyl)-1-ethyl-1H-imidazol-2-yl]-(E)-vinyl}-phenyl)-1,2,5-thiadiazolidin-3-one-1 ,1-dioxide (48 mg, 16% yield).

LCMS: m/z 477 (M+H)⁺. ¹H NMR (DMSO-d₆, 400 MHz): δ 1.37 (t, 3H), 4.05 (s, 2H), 4.24 (q, 2H), 7.07-7.17 (m, 3H), 7.46-7.52 (m, 2H), 7.61-7.68 (m, 3H), 7.93 (s, 1H), 8.25 (d, 1H) ppm.

### Example 406

4-(4-(2,4-Dichloro-phenyl)-2-{2-[4-(methoxycarbonylmethyl-amino)-phenyl]-(E)-vinyl}-imidazol-1-ylmethyl)-benzoic acid methyl ester (345 mg, 0.62 mmol)) was cyclized according to general procedure Y to provide 4-(4-(2,4-dichloro-phenyl)-2-{2-[4-(1,1,4-trioxo-1,2,5-thiadiazolidin-2-yl)-phenyl]-(E)-vinyl}-imidazol-1-ylmethyl)-benzoic acid methyl ester (59 mg, 16% yield).

LCMS: m/z 597 (M+H)⁺. ¹H NMR (DMSO-d₆, 400 MHz): δ 3.82 (s, 3H), 4.03 (s, 2H), 5.64 (s, 2H), 6.67 (d, 1H), 7.02 (d, 1H), 7.09 (d, 1H), 7.31 (d, 1H), 7.42 (d, 1H), 7.54 (d, 1H), 7.60 (d, 1H), 7.68 (m, 1H), 7.91-8.00 (m, 4H), 8.09-8.16 (m, 2H) ppm.

### Example 407

The compound of Example 406 (31 mg, 0.052 mmol) was hydrolyzed according to general procedure F to provide 4-(4-(2,4-dichloro-phenyl)-2-{2-[4-(1,1,4-trioxo-1,2,5-thiadiazolidin-2-yl)-phenyl]-(E)-vinyl}-imidazol-1-ylmethyl)-benzoic acid (16 mg, 53% yield).

LCMS: m/z 583 (M+H)⁺. ¹H NMR (DMSO-d₆, 400 MHz): δ 4.03 (s, 2H), 5.54 (s, 2H), 6.63 (d, 1H), 7.02 (d, 1H), 7.07 (d, 1H), 7.13-7.20 (m, 2H), 7.29 (d, 1H), 7.51 (m, 1H), 7.59 (d, 1H), 7.65 (m, 1H), 7.87-7.95 (m, 4H), 8.09 (d, 1H) ppm.

### Example 408

(4-{4-(2,4-Dichloro-phenyl)-2-[2-(3′-methanesulfonyl-biphenyl-4-yl)-(E)-vinyl]-imidazol-1-yl}-phenylamino)-acetic acid methyl ester (174 mg, 0.27 mmol)) was cyclized according to general procedure Y to provide 5-(4-{4-(2,4-dichloro-phenyl)-2-[2-(3′-methanesulfonyl-biphenyl-4-yl)-(E)-vinyl]-imidazol-1-yl}-phenyl)-1,2,5-thiadiazolidin-3-one-1,1-dioxide (59 mg, 32% yield).

LCMS: m/z 679 (M+H)⁺. ¹H NMR (acetone-d₆, 400 MHz): δ 3.19 (s, 3H), 4.47 (s, 2H), 6.89 (d, 1H), 7.34-7.49 (m, 6H), 7.54-7.62 (m, 3H), 7.63-7.70 (m, 3H), 7.87 (d, 1H), 7.91 (s, 1H), 7.95 (d, 1H), 8.16 (m, 1H), 8.39 (d, 1H) ppm.

### Example 409

4-{4-(2,4-Dichloro-phenyl)-2-[2-(3′-methanesulfonyl-biphenyl-4-yl)-(E)-vinyl]-imidazol-1-yl}-benzaldehyde (700 mg, 1.22 mmol)) was cyclized according to general procedure AB to provide (±)-4-(4-{4-(2,4-dichloro-phenyl)-2-[2-(3′-methanesulfonyl-biphenyl-4-yl)-(E)-vinyl]-imidazol-1-yl}-phenyl)-1,1-dioxo-1,2,5-thiadiazolidin-3-ylideneamine (210 mg, 25% yield).

LCMS: m/z 678 (M+H)⁺. ¹H NMR (DMSO-d₆, 400 MHz): δ 3.31 (s, 3H), 5.52 (d, 1H), 6.94 (d, 1H), 7.57 (dd, 1H), 7.66-7.74 (m, 7H), 7.76-7.82 (m, 3H), 7.88 (d, 1H), 7.92 (m, 1H), 8.06 (m, 1H), 8.09 (s, 1H), 8.18 (m, 1H), 8.31 (d, 1H), 8.42 (br s, 1H) ppm.

### Example 410

(4-{4-(2,4-Dichloro-phenyl)-2-[2-(3′-isopropoxycarbonylamino-biphenyl-4-yl)-(E)-vinyl]-imidazol-1-ylmethyl}-phenylamino)-acetic acid methyl ester (790 mg, 1.18 mmol)) was cyclized according to general procedure Y to provide [4′-(2-{4-(2,4-dichloro-phenyl)-1-[4-(1,1,4-trioxo-1,2,5-thiadiazolidin-2-yl)-benzyl]-1H-imidazol-2-yl]-(E)-vinyl) -biphenyl-3-yl}-carbamic acid isopropyl ester (27 mg, 3% yield). LCMS: m/z 716 (M+H)⁺.

By analagous methods to those used to prepare Example 410, the following compounds were synthesized:

| Example | Name | LC/MS (m/z) |
|---|---|---|
| 411 | [4′-(2-{4-(2,4-dichloro-phenyl)-1-[4-(1,1,4- | 730 (M + H)⁺ |
| | trioxo-1,2,5-thiadiazolidin-2-yl)-benzyl]- | |
| | 1H-imidazol-2-yl}-(E)-vinyl)- | |
| | biphenyl-3-yl]-carbamic acid isobutyl | |
| | ester | |
| 412 | 5-(4-{4-(2,4-dichloro-phenyl)-2-[2-(3′- | 657 (M + H)⁺ |
| | isopropyl-biphenyl-4-yl)-(E)-vinyl]- | |
| | imidazol-1-ylmethyl}-phenyl)-1,2,5- | |
| | thiadiazolidin-3-one-1,1-dioxide | |
| 413 | 5-(4-{4-(2,4-dichloro-phenyl)-2-[2-(3′- | 629 (M + H)⁺ |
| | methyl-biphenyl-4-yl)-(E)-vinyl]-imidazol- | |
| | 1-ylmethyl}-phenyl)-1,2,5-thiadiazolidin- | |
| | 3-one-1,1-dioxide | |
| 414 | 5-(4-{4-(2,4-dichloro-phenyl)-2-[2-(4- | 631 (M + H)⁺ |
| | phenoxy-phenyl)-(E)-vinyl]-imidazol- | |
| | 1-ylmethyl}-phenyl)-1,2,5-thiadiazolidin- | |
| | 3-one-1,1-dioxide | |

### Example 415

(4-{4-(2,4-Dichloro-phenyl)-2-[2-(3′-trifluoromethyl-biphenyl-4-yl)-(E)-vinyl]-imidazol-1-ylmethyl}-2-methyl-phenylamino)-acetic acid methyl ester (70 mg, 0.1 mmol)) was treated according to general procedure Y, parts Y3-A and Y3-B to provide (4-{4-(2,4-dichloro-phenyl)-2-[2-(3′-trifluoromethyl-biphenyl-4-yl)-(E)-vinyl]-imidazol-1-ylmethyl}-2-methyl-phenylaminosulfonamido)-acetic acid methyl ester (45 mg, 57% yield).

LCMS: m/z 729 (M+H)⁺.

### Example 416

(4-{4-(2,4-Dichloro-phenyl)-2-[2-(3′-trifluoromethyl-biphenyl-4-yl)-(E)-vinyl]-imidazol-1-ylmethyl}-2-methyl-phenylamino)-acetic acid methyl ester (70 mg, 0.1 mmol)) was cyclized according to general procedure Y to provide 5-(4-{4-(2,4-dichloro-phenyl)-2-[2-(3′-trifluoromethyl-biphenyl-4-yl)-(E)-vinyl]-imidazol-1-ylmethyl}-2-methyl-phenyl)-1,2,5-thiadiazolidin-3-one-1,1-dioxide (22 mg, 29% yield).

LCMS: m/z 697 (M+H)⁺. ¹H NMR (CD₃OD, 400 MHz): δ 2.41 (s, 3H), 4.18 (s, 2H), 5.45 (s, 2H), 7.14 (m, 1H), 7.19 (d, 1H), 7.22 (s, 1H), 7.40 (dd, 1H), 7.49 (d, 1H), 7.53 (d, 1H), 7.61 (d, 1H), 7.63-7.66 (m, 2H), 7.69 (s, 4H), 7.82 (s, 1H), 7.92 (m, 2H), 8.05 (d, 1H) ppm.

### Example 417

2-(4-{4-(2,4-Dichloro-phenyl)-2-[2-(3′-trifluoromethyl-biphenyl-4-yl)-(E)-vinyl]-imidazol-1-ylmethyl}-phenylamino)-pentanoic acid ethyl ester (100 mg, 0.14 mmol) was cyclized according to general procedure Y to provide (±)-5-(4-{4-(2,4-dichloro-phenyl)-2-[2-(3′-trifluoromethyl-biphenyl-4-yl)-(E)-vinyl]-imidazol-1-ylmethyl}-phenyl)-4-propyl-1,2,5-thiadiazolidin-3-one-1,1-dioxide (25 mg, 24% yield).

LCMS: m/z 725 (M+H)⁺. ¹H NMR (CD₃OD, 400 MHz): δ 0.90 (t, 3H), 1.41 (m, 2H), 1.85 (m, 2H), 4.66 (m, 1H), 5.38 (s, 2H), 7.10 (d, 1H), 7.25-7.33 (m, 4H), 7.37 (dd, 1H), 7.47 (d, 1H), 7.61-7.67 (m, 7H), 7.71 (s, 1H), 7.83-7.88 (m, 2H), 8.02 (d, 1H) ppm.

### Example 418

4-{4-(2,4-Dichloro-phenyl)-2-[2-(3′-trifluoromethyl-biphenyl-4-yl)-(E)-vinyl]-imidazol-1-ylmethyl}-benzaldehyde (360 mg, 0.62 mmol) was cyclized according to general procedure AB to provide (±)-4-(4-{4-(2,4-dichloro-phenyl)-2-[2-(3′-trifluoromethyl-biphenyl-4-yl)-(E)-vinyl]-imidazol-1-ylmethyl}-phenyl)-1,2,5-thiadiazolidin-3-one-1,1-dioxide (29 mg, 7% yield).

LCMS: m/z 683 (M+H)⁺. ¹H NMR (CD₃OD, 400 MHz): δ 5.06 (s, 1H), 5.36 (s, 2H), 7.04 (d, 1H), 7.24, (d, 2H), 7.35 (dd, 1H), 7.46 (d, 1H), 7.53 (d, 2H), 7.59-7.66 (m, 7H), 7.68 (s, 1H), 7.85 (m, 2H), 8.03 (d, 1H) ppm.

### Example 419

4-[2-[2-(4-Bromo-phenyl)-(E)-vinyl]-4-(2,4-dichloro-phenyl)-imidazol-1-ylmethyl]-benzoic acid methyl ester (1088 mg, 2.0 mmol) was coupled with 2-fluoro-5-propoxyphenyl boronic acid according to general procedure B to provide 4-{4-(2,4-dichloro-phenyl)-2-[2-(2′-fluoro-5′-propoxy-biphenyl-4-yl)-(E)-vinyl]-imidazol-1-ylmethyl}-benzoic acid methyl ester (1120 mg, 91% yield).

LCMS: m/z 615 (M+H)⁺.

### Example 420

4-{4-(2,4-Dichloro-phenyl)-2-[2-(2′-fluoro-5′-propoxy-biphenyl-4-yl)-(E)-vinyl]-imidazol-1-ylmethyl}-benzoic acid methyl ester (983 mg, 1.6 mmol) was hydrolyzed according to general procedure F to provide 4-{4-(2,4-dichloro-phenyl)-2-[2-(2′-fluoro-5′-propoxy-biphenyl-4-yl)-(E)-vinyl]-imidazol-1-ylmethyl}-benzoic acid (722 mg, 75% yield).

LCMS: m/z 601 (M+H)⁺. ¹H NMR (DMSO-d₆, 400 MHz): δ 1.00 (t, 3H), 1.74 (m, 2H), 3.99 (t, 2H), 5.67 (s, 2H), 6.93-6.98 (m, 1H), 7.03-7.07 (m, 1H), 7.20-7.26 (m, 1H), 7.35-7.41 (m, 3H), 7.52 (dd, 1H), 7.56-7.62 (m, 3H), 7.66 (d, 1H), 7.77 (d, 2H), 7.95 (d, 2H), 8.13 (s, 1H), 8.30 (d, 1H) ppm.

### Example 421

4-[2-[2-(4-Bromo-phenyl)-(E)-vinyl]-4-(2,4-dichloro-phenyl)-imidazol-1-ylmethyl]-benzoic acid methyl ester (1088 mg, 2.0 mmol) was coupled with 3,4-difluorophenyl boronic acid according to general procedure B to provide 4-{4-(2,4-dichloro-phenyl)-2-[2-(3′,4′-difluoro-biphenyl-4-yl)-(E)-vinyl]-imidazol-1-ylmethyl}-benzoic acid methyl ester (960 mg, 83% yield).

LCMS: m/z 575 (M+H)⁺.

### Example 422

4-{4-(2,4-Dichloro-phenyl)-2-[2-(3′,4′-difluoro-biphenyl-4-yl)-(E)-vinyl]-imidazol-1-ylmethyl}-benzoic acid methyl ester (876 mg, 1.52 mmol) was hydrolyzed according to general procedure F to provide 4-{4-(2,4-Dichloro-phenyl)-2-[2-(3′,4′-difluoro-biphenyl-4-yl)-(E)-vinyl]-imidazol-1-ylmethyl}-benzoic acid (768 mg, 86% yield).

LCMS: m/z 561 (M+H)⁺.

### Example 423

4-[4-(2,4-Dichloro-phenyl)-2-(4′-hydroxy-biphenyl-4-ylmethyl)-imidazol-1-ylmethyl]-benzoic acid methyl ester (200 mg, 0.37 mmol) was coupled with 4-trifluoromethylphenyl boronic acid according to general procedure W to give 4-{4-(2,4-dichloro-phenyl)-2-[4′-(4-trifluoromethyl-phenoxy)-biphenyl-4-ylmethyl]-imidazol-1-ylmethyl}-benzoic acid methyl ester (27 mg, 11% yield).

LCMS: m/z 687 (M+H)⁺. ¹H NMR (CD₃OD, 400 MHz): δ 3.86 (s, 3H), 4.18 (s, 2H), 5.17 (s, 2H), 7.02 (d, 2H), 7.09-7.15 (m, 4H), 7.18 (d, 2H), 7.37 (dd, 1H), 7.43 (d, 2H), 7.47 (d, 1H), 7.52-7.57 (m, 2H), 7.61 (m, 2H), 7.67 (s, 1H), 7.89 (m, 2H), 8.02 (d, 1H) ppm.

### Example 424

4-{4-(2,4-dichloro-phenyl)-2-[4′-(4-methanesulfonyl-phenoxy)-biphenyl-4-ylmethyl]-imidazol-1-ylmethyl}-benzoic acid methyl ester was prepared by analagous methods to those used to prepare Example 423. LCMS: m/z 697 (M+H)⁺

### Example 425

4-{4-(2,4-Dichloro-phenyl)-2-[4′-(4-trifluoromethyl-phenoxy)-biphenyl-4-ylmethyl]-imidazol-1-ylmethyl}-benzoic acid methyl ester (25 mg, 0.036 mmol) was hydrolyzed according to general procedure F to provide 4-{4-(2,4-dichloro-phenyl)-2-[4′-(4-trifluoromethyl-phenoxy)-biphenyl-4-ylmethy]-imidazol-1-ylmethyl}-benzoic acid (23 mg, 94% yield). LCMS: m/z 673 (M+H)⁺.

### Example 426

4-{4-(2,4-dichloro-phenyl)-2-[4′-(4-methanesulfonyl-phenoxy)-biphenyl-4-ylmethyl]-imidazol-1-ylmethyl}-benzoic acid was prepared by analagous methods to those used to prepare Example 425. LCMS: m/z 683 (M+H)⁺.

### Example 427

4-[4-(2,4-Dichloro-phenyl)-2-(4′-hydroxy-biphenyl-4-yloxymethyl)-imidazol-1-ylmethyl]-benzoic acid methyl ester (55 mg, 0.1 mmol) was reacted with 4-fluorobenzotrifluoride according to general procedure I. The resulting trifluoromethyl phenyl ether was hydrolyzed according to general procedure F to provide 4-{4-(2,4-dichloro-phenyl)-2-[4′-(4-trifluoromethyl-phenoxy)-biphenyl-4-yloxymethyl]-imidazol-1-ylmethyl}-benzoic acid (5 mg, 7% yield).

LCMS: m/z 689 (M+H)⁺. ¹H NMR (DMSO-d₆, 400 MHz): δ 5.24 (s, 2H), 5.50 (s, 2H), 7.03 (m, 2H), 7.19 (d, 4H), 7.33 (d, 2H), 7.49 (dd, 1H), 7.59 (m, 2H), 7.65 (d, 1H), 7.69 (m, 2H), 7.75 (d, 2H), 7.91 (m, 2H), 8.07 (s, 1H), 8.17 (d, 1H) ppm.

### Example 428

4-[2-(4-Amino-benzyl)-4-(2,4-dichloro-phenyl)-imidazol-1-ylmethyl]-benzoic acid methyl ester (77 mg, 0.16 mmol) was coupled with 3-acetylbenzenesulfonyl chloride according to general procedure L. The resulting sulfonamide was hydrolyzed according to general procedure F to give 4-[2-[4-(3-acetyl-benzenesulfonylamino)-benzyl]-4-(2,4-dichloro-phenyl)-imidazol-1-ylmethyl]-benzoic acid (84 mg, 80% yield).

LCMS: m/z 634 (M+H)⁺.

### Example 429

4-[2-(4-Amino-benzyl)-4-(2,4-dichloro-phenyl)-imidazol-1-ylmethyl]-benzoic acid methyl ester (77 mg, 0.16 mmol) was coupled with 2,5-dimethoxybenzenesulfonyl chloride according to general procedure L. The resulting sulfonamide was hydrolyzed according to general procedure F to give 4-{4-(2,4-dichloro-phenyl)-2-[4-(2,5-dimethoxy-benzenesulfonylamino)-benzyl]-imidazol-1-ylmethyl}-benzoic acid (74 mg, 69% yield).

LCMS: m/z 652 (M+H)⁺.

### Example 430

4-[2-[4-(3-acetyl-benzenesulfonylamino)-benzyl]-4-(2,4-dichloro-phenyl)-imidazol-1-ylmethyl]-benzoic acid (84 mg, 0.13 mmol) was alkylated with methyl iodide (2 eq) according to general procedure P, and the resulting methyl ester was hydrolyzed according to general procedure F to give 4-[2-{4-[(3-acetyl-benzenesulfonyl)-methyl-amino]-benzyl}-4-(2,4-dichloro-phenyl)-imidazol-1-ylmethyl]-benzoic acid (35 mg, 42% yield).

LCMS: m/z 648 (M+H)⁺. ¹H NMR (DMSO-d₆, 400 MHz): δ 2.55 (s, 3H), 3.08 (s, 3H), 4.07 (s, 2H), 5.34 (s, 2H), 6.94 (d, 2H), 7.09-7.17 (m, 4H), 7.46 (dd, 1H), 7.62 (d, 1H), 7.67-7.74 (m, 2H), 7.84 (d, 2H), 7.89 (m, 1H), 7.95 (s, 1H), 8.17 (dd, 1H), 8.25 (m, 1H) ppm.

By analagous methods to those used to prepare Example 430, the following compounds were synthesized:

| Example | Name | LC/MS (m/z) |
|---|---|---|
| 431 | 4-(4-(2,4-dichloro-phenyl)-2-{4-[(2,5- | 666 (M + H)⁺ |
| | dimethoxy-benzenesulfonyl)-methyl- | |
| | amino]-benzyl}-imidazol-1- | |
| | ylmethyl)-benzoic acid | |
| 432 | 4-(4-(2,4-dichloro-phenyl)-2-{4-[(3,4- | 666 (M + H)⁺ |
| | dimethoxy-benzenesulfonyl)-methyl- | |
| | amino]-benzyl}-imidazol-1- | |
| | ylmethyl)-benzoic acid | |

### Example 433

5-[2-(4-Bromo-benzyl)-4-(2,4-dichloro-phenyl)-imidazol-1-yl]-2-methanesulfonyl-benzoic acid methyl ester (420 mg, 0.7 mmol) was coupled with 4-hydroxyphenyl boronic acid according to general procedure B to provide 5-[4-(2,4-dichloro-phenyl)-2-(4′-hydroxy-biphenyl-4-ylmethyl)-imidazol-1-yl]-2-methanesulfonyl-benzoic acid methyl ester. The phenol intermediate was coupled with 4-trifluoromethylphenyl boronic acid according to general procedure W to give the trifluoromethylphenyl phenyl ether intermediate, which was hydrolyzed according to general procedure F to afford 5-{4-(2,4-dichloro-phenyl)-2-[4′-(4-trifluoromethyl-phenoxy)-biphenyl-4-ylmethyl]-imidazol-1-yl}-2-methanesulfonyl-benzoic acid (37 mg, 7% yield).

LCMS: m/z 737 (M+H)⁺.

### Example 434

4′-[4-(2,4-Dichloro-phenyl)-1-ethyl-1H-imidazol-2-ylmethyl]-biphenyl-4-ol (100 mg, 0.24 mmol) was treated with methyl bromoacetate according to general procedure E. The phenyl O-acetyl ester product was hydrolyzed according to general procedure F to afford {4′-[4-(2,4-dichloro-phenyl)-1-ethyl-1H-imidazol-2-ylmethyl]-biphenyl-4-yloxy}-acetic acid (18 mg, 16% yield).

LCMS: m/z 481 (M+H)⁺.

### Example 435

4′-[4-(2,4-Dichloro-phenyl)-1-ethyl-1H-imidazol-2-ylmethyl]-biphenyl-4-ol (100 mg, 0.24 mmol) was treated with ethyl bromo(4-fluorophenyl)acetate according to general procedure E. The phenyl O-acetyl ester product was hydrolyzed according to general procedure F to afford {4′-[4-(2,4-dichloro-phenyl)-1-ethyl-1H-imidazol-2-ylmethyl]-biphenyl-4-yloxy}-(4-fluoro-phenyl)-acetic acid (34 mg, 25% yield).

LCMS: m/z 575 (M+H)⁺. ¹H NMR (DMSO-d₆, 400 MHz): δ 1.20 (t, 3H), 3.96 (q, 2H), 4.16 (s, 2H), 5.60 (s, 1H), 6.99 (d, 2H), 7.14-7.21 (m, 3H), 7.29 (d, 2H) 7.44 (dd, 1H), 7.51-7.62 (m, 6H), 7.84 (s, 1H), 8.17 (d, 1H) ppm.

### Example 436

4-[2-(4-Bromo-benzyl)-4-(2,4-dichloro-phenyl)-imidazol-1-yl]methanesulfonyl amino-benzoic acid methyl ester (305 mg, 0.5 mmol) was treated as described in general procedure B using 4-isobutyl butyl phenylboronic acid (102 mg, 0.57 mmol) to give 4-[2-(4′-Isobutyl-biphenyl-4-ylmethyl)-4-(2,4-dichloro-phenyl)-imidazol-1-yl]-2-methanesulfonylamino-benzoic acid methyl ester (219 mg, 66% yield). 4-[2-(4′-Isobutyl-biphenyl-4-ylmethyl)-4-(2,4-dichloro-phenyl)-imidazol-1-yl]-2-methanesulfonylamino-benzoic acid methyl ester (67 mg, 0.1 mmol) was hydrolyzed following general procedure F to give 4-[2-(4′-Isobutyl-biphenyl-4-ylmethyl)-4-(2,4-dichloro-phenyl)-imidazol-1-yl]-2-methanesulfonylamino-benzoic acid (56 mg, 86% yield).

LCMS: m/z 649 (M+H)⁺; ¹H NMR (DMSO-d6, 400 MHz): d 0.82 (d, 6H), 1.81 (m 1H), 2.43 (d, 2H), 3.45 (s, 3H), 4.17 (s, 2H), 6.96 (d, 1H), 7.10 (d, 2H), 7.18 (d, 1H), 7.35 (d, 2H), 7.46 (s, 1H), 7.47-7.50 (m, 2H), 7.56 (d, 2H), 7.95 (s, 1H), 8.04 (d, 2H), 8.19 (d, 2H) ppm.

### Example 437

4-[2-(4-Bromo-benzyl)-4-(2,4-dichloro-phenyl)-imidazol-1-yl]methanesulfonyl amino-benzoic acid methyl ester (305 mg, 0.5 mmol) was treated as described in general procedure B using 3-isopropyl butyl phenylboronic acid (95 mg, 0.57 mmol) to give 4-[2-(3′-Isopropyl-biphenyl-4-ylmethyl)-4-(2,4-dichloro-phenyl)-imidazol-1-yl]-2-methanesulfonylamino-benzoic acid methyl ester (219 mg, 66% yield). 4-[2-(3′-Isopropyl-biphenyl-4-ylmethyl)-4-(2,4-dichloro-phenyl)-imidazol-1-yl]-2-methanesulfonylamino-benzoic acid methyl ester (67 mg, 0.1 mmol) was hydrolyzed following general procedure F to give 4-[2-(3′-Isopropyl-biphenyl-4-ylmethyl)-4-(2,4-dichloro-phenyl)-imidazol-1-yl]-2-methanesulfonylamino-benzoic acid (56 mg, 86% yield).

LCMS: m/z 635 (M+H)⁺; ¹H NMR (DMSO-d6, 400 MHz): d 1.21 (d, 6H), 2.82 (m, 1H), 3.36 (s, 3H), 4.15 (s, 2H), 6.95 (d, 1H), 7.13 (d, 2H), 7.19 (d, 1H), 7.31 (d, 2H), 7.35-7.39 (m, 1H), 7.43 (s, 1H), 7.47-7.51 (m, 1H), 7.64 (d, 2H), 7.96 (s, 1H), 8.04 (d, 2H), 8.18 (d, 2H) ppm.

### Example 438

4′-[4-(2,4-Dichloro-phenyl)-1-ethyl-1H-imidazol-2-ylmethyl]-biphenyl-4-ol (44 mg, 0.1 mmol) was treated with 4-bromo-2-tert-butoxycarbonylamino-butyric acid methyl ester according to general procedure E. The resulting α-N-Boc-amino ester was deprotected according to general procedure O, then treated with methanesulfonyl chloride according to general procedure L. The resulting methanesulfonamide was hydrolyzed according to general procedure F to provide 4-{4′-[4-(2,4-dichloro-phenyl)-1-ethyl-1H-imidazol-2-ylmethyl]-biphenyl-4-yloxy}-2-(S)-methanesulfonylamino-butyric acid (30 mg, 48% yield).

LCMS: m/z 602 (M+H)⁺. ¹H NMR (CD₃OD, 400 MHz): d 1.30 (t, 3H), 2.10-2.18 (m, 1H), 2.38-2.47 (m, 1H), 2.97 (s, 3H), 3.93 (q, 2H), 4.16-4.22 (m, 2H), 4.24 (s, 2H), 4.30 (dd, 1H), 7.00 (m, 2H), 7.25 (d, 2H), 7.35 (dd, 1H), 7.47 (d, 1H), 7.49-7.54 (m, 4H), 7.62 (s, 1H), 7.94 (d, 1H) ppm.

### Example 439

4′-[4-(2,4-Dichloro-phenyl)-1-ethyl-1H-imidazol-2-ylmethyl]-biphenyl-4-ol (87 mg, 0.2 mmol) was treated with 4-bromo-2-tert-butoxycarbonylamino-butyric acid methyl ester according to general procedure E. The resulting α-N-Boc-amino ester was deprotected according to general procedure O, then treated with trifluoromethanesulfonic anhydride according to general procedure L. The trifluoromethanesulfonamide was then hydrolyzed according to general procedure F to provide 4-{4′-[4-(2,4-dichloro-phenyl)-1-ethyl-1H-imidazol-2-ylmethyl]-biphenyl-4-yloxy}-2-(S)-trifluoromethanesulfonylamino-butyric acid (52 mg, 39% yield).

LCMS: m/z 656 (M+H)⁺. ¹H NMR (CD₃OD, 400 MHz): d 1.28 (t, 3H), 2.12-2.22 (m, 1H), 2.39-2.48 (m, 1H), 3.94 (q, 2H), 4.12-4.18 (m, 2H), 4.25 (s, 2H), 4.38 (dd, 1H), 6.99 (m, 2H), 7.25 (d, 2H), 7.35 (dd, 1H), 7.48 (d, 1H), 7.49-7.54 (m, 4H), 7.63 (s, 1H), 7.92 (d, 1H) ppm.

### Example 440

To a solution of 4′-[4-(2,4-dichloro-phenyl)-1-ethyl-1H-imidazol-2-ylmethyl]-biphenyl-4-ol (564 mg, 1.33 mmol), (2S,4R)-4-hydroxy-piperidine-1,2-dicarboxylic acid 1-tert-butyl ester 2-methyl ester (231 mg, 0.89 mmol), and triphenylphosphine (466 mg, 1.78 mmol) in 1 ml dry THF was added diisopropyl azodicarboxylate (363 mg, 1.78 mmol) dropwise while sonicating. The mixture was then sonicated 3.5 hours (bath at 50° C.). The solvent was evaporated in vacuo and the residue was purified by flash column chromatography to afford 127 mg of 4(S)-{4′-[4-(2,4-dichloro-phenyl)-1-ethyl-1H-imidazol-2-ylmethyl]-biphenyl-4-yloxy}-piperidine-1,2(S)-dicarboxylic acid 1-tert-butyl ester 2-methyl ester. The 4-N-Boc-amino ester was deprotected according to general procedure O, then treated with trifluoromethanesulfonic anhydride according to general procedure L. The trifluoromethanesulfonamide was then hydrolyzed according to general procedure F to provide 4(S)-{4′-[4-(2,4-dichloro-phenyl)-1-ethyl-1H-imidazol-2-ylmethyl]-biphenyl-4-yloxy}-1-trifluoromethanesulfonyl-piperidine-2-(S)-carboxylic acid (17 mg, 2% yield).

LCMS: m/z 682 (M+H)⁺. ¹H NMR (CD₃OD, 400 MHz): d 1.27 (t, 3H), 1.62-1.80 (m, 2H), 2.18 (d, 1H), 2.91 (d, 1H), 3.75 (m, 1H), 3.87-3.96 (m, 3H), 4.21 (s, 2H), 4.40 (m, 1H), 4.62 (br s, 1H), 6.98 (d, 2H), 7.24 (d, 2H), 7.34 (dd, 1H), 7.45-7.51 (m, 5H), 7.61 (s, 1H), 7.94 (d, 1H) ppm.

### Example 441

2-Amino-5-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using methyl chloroformate (39 mL, 0.5 mmol) and DIEA (88 mL, 0.5 mmol) till the starting material disappeared (monitored by LC-MS). The resulted 5-{4′-[4-(2,4-dichloro-phenyl)-1-ethyl-1H-imidazol-2-ylmethyl]-biphenyl-4-yloxy}-2-methoxycarbonylamino-benzoic acid methyl ester was concentrated and treated directly as described in general procedure F to give 5-{4′-[4-(2,4-dichloro-phenyl)-1-ethyl-1H-imidazol-2-ylmethyl]-biphenyl-4-yloxy}-2-methoxycarbonylamino-benzoic acid (50 mg, 41% yield).

LCMS: m/z 616 (M+H)⁺; ¹H NMR (DMSO-d6, 400 MHz): d 1.19 (t, 3H), 3.79 (s, 3H), 3.98 (q, 2H), 4.17 (s, 2H), 7.01 (d, 2H), 7.13 (dd, 1H), 7.31 (d, 2H), 7.43 (dd, 1H), 7.57-7.65 (m, 6H), 7.83 (s, 1H), 8.16 (d, 1H), 8.20 (d, 1H) ppm.

### Example 442

2-Amino-5-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using ethyl chloroformate (48 mL, 0.5 mmol) and DIEA (88 mL, 0.5 mmol) till the starting material disappeared (monitored by LC-MS). The resulted 5-{4′-[4-(2,4-dichloro-phenyl)-1-ethyl-1H-imidazol-2-ylmethyl]-biphenyl-4-yloxy}-2-ethoxycarbonylamino-benzoic acid methyl ester was concentrated and treated directly as described in general procedure F to give 5-{4′-[4-(2,4-dichloro-phenyl)-1-ethyl-1H-imidazol-2-ylmethyl]-biphenyl-4-yloxy}-2-ethoxycarbonylamino-benzoic acid (67 mg, 53% yield).

LCMS: m/z 630 (M+H)⁺; ¹H NMR (DMSO-d6, 400 MHz): d 1.18 (t, 3H), 1.22 (t, 3H), 3.97 (q, 2H), 4.10 (q, 2H), 4.17 (s, 2H), 7.00 (d, 2H), 7.13 (dd, 1H), 7.31 (d, 2H), 7.43 (dd, 1H), 7.57-7.66 (m, 6H), 7.83 (s, 1H), 8.16 (d, 1H), 8.21 (d, 1H) ppm.

### Example 443

2-Amino-5-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using ethyl oxalyl chloride (56 mL, 0.5 mmol) and DIEA (88 mL, 0.5 mmol) to give 5-{4′-[4-(2,4-dichloro-phenyl)-1-ethyl-1H-imidazol-2-ylmethyl]-biphenyl-4-yloxy}-2-(ethoxyoxalyl-amino)-benzoic acid methyl ester, which was treated as described in general procedure F (starting with pure ester, no column needed after the reaction work-up. The white-powder product was triturated several times with ether) to give 5-{4′-[4-(2,4-dichloro-phenyl)-1-ethyl-1H-imidazol-2-ylmethyl]-biphenyl-4-yloxy}-2-(oxalyl-amino)-benzoic acid (59 mg, 47% yield).

LCMS: m/z 630 (M+H)⁺; ¹H NMR (DMSO-d6, 400 MHz): d 1.19 (t, 3H), 3.98 (q, 2H), 4.20 (s, 2H), 7.11 (d, 2H), 7.34 (d, 2H), 7.42-7.46 (m, 2H), 7.60-7.63 (m, 4H), 7.68 (d, 2H), 7.86 (s, 1H), 8.14 (d, 1H), 8.65 (d, 1H) ppm.

### Example 444

2-Amino-5-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using tert-butylacetyl chloride (70 mL, 0.5 mmol) and DIEA (88 mL, 0.5 mmol) to give 5-{4′-[4-(2,4-dichloro-phenyl)-1-ethyl-1H-imidazol-2-ylmethyl]-biphenyl-4-yloxy}-2-(3,3-dimethyl-butyrylamino)-benzoic acid methyl ester, which was treated as described in general procedure F to give 5-{4′-[4-(2,4-dichloro-phenyl)-1-ethyl-1H-imidazol-2-ylmethyl]-biphenyl-4-yloxy}-2-(3,3-dimethyl-butyrylamino)-benzoic acid (63 mg, 48% yield).

LCMS: m/z 656 (M+H)⁺; ¹H NMR (DMSO-d6, 400 MHz): d 1.02 (s, 9H), 1.17 (t, 3H), 2.21 (s, 2H), 3.96 (q, 2H), 4.16 (s, 2H), 7.06 (d, 2H), 7.30-7.35 (m, 3H), 7.42 (dd, 1H), 7.52 (d, 1H), 7.58-7.60 (m, 3H), 7.64 (d, 2H), 7.83 (s, 1H), 8.14 (d, 1H), 8.48 (d, 1H) ppm.

### Example 445

2-Amino-5-{4′-[4-(2,4-dichloro-phenyl)-1-ethyl-1H-imidazol-2-ylmethyl]-biphenyl-4-yloxy}-benzoic acid methyl ester (115 mg, 0.2 mmol) was treated as described in general procedure L using hexanoyl chloride (70 mL, 0.5 mmol) and DIEA (88 mL, 0.5 mmol) to give 5-{4′-[4-(2,4-dichloro-phenyl)-1-ethyl-1H-imidazol-2-ylmethyl]-biphenyl-4-yloxy}-2-hexanoylamino-benzoic acid methyl ester, which was treated as described in general procedure F to give 5-{4′-[4-(2,4-dichloro-phenyl)-1-ethyl-1H-imidazol-2-ylmethyl]-biphenyl-4-yloxy}-2-hexanoylamino-benzoic acid (67 mg, 51% yield).

LCMS: m/z 656 (M+H)⁺; ¹H NMR (DMSO-d6, 400 MHz): d 0.91 (t, 3H), 1.17 (t, 3H), 1.42 (m, 4H), 1.71 (m, 2H), 2.22 (t, 2H), 3.96 (q, 2H), 4.16 (s, 2H), 7.06 (d, 2H), 7.29-7.34 (m, 3H), 7.42 (dd, 1H), 7.51 (d, 1H), 7.56-7.59 (m, 3H), 7.64 (d, 2H), 7.83 (s, 1H), 8.14 (d, 1H), 8.46 (d, 1H) ppm.

### Biological Assay

The following assay methods are utilized to identify compounds of formula I which are effective in inhibiting the activity of certain phosphatases, an example of which, as used herein, is PTP1B.

### PTP1B Assay

The assay for PTP1B inhibition is based on the detection of the complex between Malachite Green dye and free phosphate, liberated from the phosphopeptide substrate by PTPase action. To each well of a flat-bottom assay plate is added 45 μL assay buffer [−50 mM Imidazole, pH 7.2, 100 mM NaCl, 5 mM DTT, and 1 mM EDTA] and 10 μL of peptide substrate (Tyrosine Phosphopeptide-1, END(ₚY)INASL (SEQ ID NO: 1), 80 μM FAC, Promega Cat # V256A) to a total volume of 55 μL. Test compound (10 μL in up to 50% DMSO) is then added. The mixture is incubated for 5 min, at 25° C., and 10 μL of PTP-1B (Protein Tyrosine Phosphatase 1B (PTP-1B); FAC 0.8 nM; Upstate Biotechnology, Cat # 14-109 lot # 19045) is then added. The mixture is incubated for 30 min at 25° C. Subsequently, 25 μL of Malachite Green reagent (10% (w/v) Ammonium Molybdate in water, Sigma Cat # A-7302, 0.2% (w/v) Malachite Green in 4 N HCl, Aldrich Cat # 21, 302-0) is then added. After incubation for 15 min at 27° C., the reaction endpoint is measured at 640 nM.

The Malachite Green reagent is prepared by mixing one volume of 10% Ammonium Molybdate with 3 volumes of 0.2% Malachite Green solution, stirring at room temperature for 30 min and then filtering and collecting the filtrate. The Malachite Green reagent is treated with 10 μL of 5% Tween 20 per 990 μL of dye solution before use.

Test compounds are typically examined at six concentrations in the above assay. For this assay, the IC50 (μM) of the enzyme inhibition assay represents the concentration of compound at which 50% signal has been inhibited.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness of the subject being treated for PTPase-mediated disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

What is claimed is:

1. A compound of Formula (I): 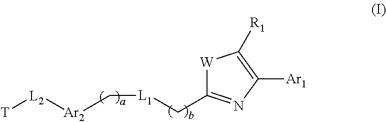
wherein
a and b are, independently, equal to 0, 1, or 2, wherein the values of 0, 1, and 2 represent a direct bond, —CH₂—, and —CH₂CH₂—, respectively, and wherein the —CH₂— and —CH₂CH₂— groups are optionally substituted 1 to 2 times with a substituent independently selected from the group consisting of: -alkyl, -aryl, -alkylene-aryl, -arylene-alkyl, -alkylene-arylene-alkyl, —O-alkyl, —O-aryl, and -hydroxyl;
W is —N(R₂)—,
wherein
R₂ is
a) -L₃-D₁-arylene-G₁-G₂;
b) -L₃-D₁-arylene-alkylene-G₁-G₂; or
c)-L₃-D₁-arylene-D₂-G₁-G₂;
wherein
L₃ is a direct bond;
D₁ is a direct bond;
D₂ is selected from the group consisting of a direct bond, —CH₂—, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, —N(R₅)SO₂N(R₆)—, —N═N—, and —N(R₅)—N(R₆)—;
wherein
R₅ and R₆ are independently selected from the group consisting of: -hydrogen, -alkyl, -aryl, -arylene-alkyl, -alkylene-aryl, and -alkylene-arylene-alkyl; and
G₁ is a direct bond, -alkylene, -alkenylene, or alkynylene; and
G₂ is —SO₃H, —P(O)(OH)₂, —P(O)(O-alkyl)(OH), —CO₂H, —CO₂-alkyl, or an acid isostere,
R₁ is
a) -hydrogen;
b) -fluoro;
c) -chloro;
d) -bromo;
e) -iodo;
f) -cyano;
g)-alkyl;
h) -aryl;
i) -alkylene-aryl;
j)-heteroaryl;
k) -alkylkene-heteroaryl;
l) -cycloalkyl;
m) -alkylene-cycloalkyl
n) -heterocyclyl; or
o) -alkylene-heterocyclyl;
L₁ is 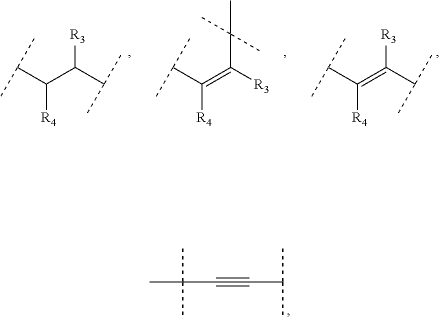
or —CH₂—, wherein R₃ and R₄ are independently selected from the group consisting of: hydrogen, chloro, fluoro, bromo, alkyl, aryl, -alkylene-aryl, -cycloalkyl, -alkylene-cycloalkyl, -heterocyclyl, -alkylene-heterocyclyl, and -alkynylene;
Ar₁ is an aryl, or fused cycloalkylaryl, wherein Ar₁ is optionally substituted 1 to 7 times with substituent(s) independently selected from the group consisting of:
a) -fluoro;
b)-chloro;
c) -bromo;
d) -iodo;
e) -cyano;
f) -nitro;
g) -perfluoroalkyl;
h) -J-R₁₄;
i) -alkyl;
j) -aryl;
k) -heteroaryl;
l) -heterocyclyl;
m) -cycloalkyl;
n) -L₅-aryl;
o) -L₅-arylene-aryl;
p) -L₅-arylene-alkyl;
q) -arylene-alkyl;
r) -arylene-arylene-alkyl;
s) -J-alkyl;
t) -J-aryl;
u) -J-alkylene-aryl;
v)-J-arylene-alkyl;
w) -J-alkylene-arylene-aryl;
x) -J-arylene-arylene-aryl;
y) -J-alkylene-arylene-alkyl;
z) -L₅-J-alkylene-aryl;
aa) -arylene-J-alkyl;
bb) -L₅-J-aryl;
cc) -L₅-J-heteroaryl;
dd) -L₅-J-cycloalkyl;
ee) -L₅-J-heterocyclyl;
ff) -L₅-J-arylene-alkyl;
gg) -L₅-J-alkylene-arylene-alkyl;
hh) -L₅-J-alkyl;
ii) -L₅-J-R₁₄;
jj) -arylene-J-R₁₄; and
ll) -hydrogen;
wherein
L₅ is a direct bond, -alkylene, -alkenylene, or -alkynylene; and
J is a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—,
wherein R₁₄, R₁₅, and R₁₆ are independently selected from the group consisting of: -hydrogen, -alkyl, -aryl, -arylene-alkyl, -alkylene-aryl, and -alkylene-arylene-alkyl;
Ar₂ is an arylene, or fused arylcycloalkylene, wherein Ar₂ is optionally substituted 1 to 7 times with substituent(s) independently selected from the group consisting of:
a) -fluoro;
b) -chloro;
c) -bromo;
d) -iodo;
e) -cyano;
f) -nitro;
g) -perfluoroalkyl;
h) -Q-R₁₇;
i) -alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s) -Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y) -Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L₆-Q-arylene-alkyl;
gg) -L₆-Q-alkylene-arylene-alkyl;
hh) -L₆-Q-alkyl;
ii) -L₆-Q-alkylene-aryl-R₁₇;
jj) -L₆-Q-alkylene-heteroaryl-R₁₇;
kk) -arylene-Q-alkylene-R₁₇;
ll) -heteroarylene-Q-alkylene-R₁₇;
mm) -L₆-Q-aryl-R₁₇;
nn) -L₆-Q-heteroarylene-R₁₇;
oo) -L₆-Q-heteroaryl-R₁₇;
pp) -L₆-Q-cycloalkyl-R₁₇;
qq) -L₆-Q-heterocyclyl-R₁₇;
rr) -L₆-Q-arylene-alkyl-R₁₇;
ss) -L₆-Q-heteroarylene-alkyl-R₁₇;
tt) -L₆-Q-alkylene-arylene-alkyl-R₁₇;
uu) -L₆-Q-alkylene-heteroarylene-alkyl-R₁₇;
vv) -L₆-Q-alkylene-cycloalkylene-alkyl-R₁₇;
ww) -L₆-Q-alkylene-heterocyclylene-alkyl-R₁₇;
xx) -L₆-Q-alkyl-R₁₇;
yy) -L₆-Q-R₁₇;
zz) -arylene-Q-R₁₇;
aaa) -heteroarylene-Q-R₁₇;
bbb) -heterocyclylene-Q-R₁₇;
ccc) -Q-alkylene-R₁₇;
ddd) -Q-arylene-R₁₇;
eee) -Q-heteroarylene-R₁₇;
fff) -Q-alkylene-arylene-R₁₇;
ggg) -Q-alkylene-heteroarylene-R₁₇;
hhh) -Q-heteroarylene-alkylene-R₁₇;
iii) -Q-arylene-alkylene-R₁₇;
jjj) -Q-cycloalkylene-alkylene-R₁₇;
kkk) -Q-heterocyclylene-alkylene-R₁₇
lll) -Q-alkylene-arylene-alkyl-R₁₇;
mmm) -Q-alkylene-heteroarylene-alkyl-R₁₇; 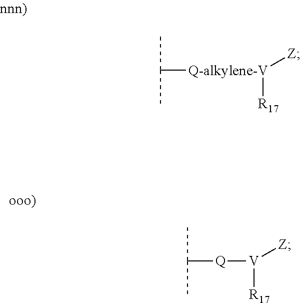
and
ppp) -hydrogen
wherein
L₆ is a direct bond, -alkylene, -alkenylene, or -alkynylene;
Q is a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₈)SO₂N(R₁₉)—, —N═N—, or —N(R₁₈)—N(R₁₉)—;
wherein
R₁₈ and R₁₉ are independently selected from the group consisting of: -hydrogen, -alkyl, -aryl, -arylene-alkyl, -alkylene-aryl, and -alkylene-arylene-alkyl;
V is 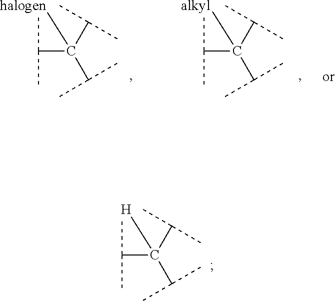
Z is hydrogen, -alkylene-aryl, -alkyl, -aryl, -heteroaryl, -heterocyclyl, -cycloalkyl, -alkylene-heteroaryl, or -alkylene-cycloalkyl;
R₁₇ is —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, acyloxy-alkylene-, or -alkylene-arylene-alkyl;
L₂ is: —CH₂—, —O—, —K—, -alkylene-, -alkenylene-, -alkynylene-, —K-alkylene-, -alkylene-K—, -alkylene-K-alkylene-, -alkenylene-K-alkylene-, -alkylene-K-alkenylene-, -arylene-K-alkylene-, alkylene-K-arylene-, -heteroarylene-K-alkylene-, alkylene-K-heteroarylene-, -arylene-K—, —K-arylene-, -heteroarylene-K—, —K-heteroarylene-, or a direct bond,
wherein
K is a direct bond, —O—, —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂—, —SO₂N(R₂₀)—, —C(O)—O—, —O—C(O)—, —S—, —S(O)—, —S(O₂)—, —N(R₂₀)SO₂N(R₂₁)—, —N═N—, or —N(R₂₀)—N(R₂₁)—;
wherein
R₂₀ and R₂₁ are independently selected from the group consisting of: -hydrogen, -alkyl, -aryl, -arylene-alkyl, -alkylene-aryl, and -alkylene-arylene-alkyl;
T is: hydrogen, alkyl, cycloalkyl, heterocyclyl, aryl, heteroaryl, fused cycloalkylaryl, fused cycloalkylheteroaryl, fused heterocyclylaryl, or fused heterocyclylheteroaryl group, wherein T is optionally substituted 1 to 7 times with substituent(s) independently selected from the group consisting of:
a) -fluoro;
b) -chloro;
c) -bromo;
d) -iodo;
e) -cyano;
f) -nitro;
g) -perfluoroalkyl;
h) —U₁-perfluoroalkyl;
i) —U₁—R₂₂;
j) -alkyl;
k) -aryl;
l) -heteroaryl;
m) -heterocyclyl;
n) -cycloalkyl;
o) -L₇-aryl;
p) -L₇-arylene-aryl;
q) -L₇-arylene-alkyl;
r) -arylene-alkyl;
s) -arylene-arylene-alkyl;
t) —U₁-alkyl;
u) —U₁-aryl;
v) —U₁-alkylene-aryl;
w) —U₁-arylene-alkyl;
x) —U₁-alkylene-arylene-aryl;
y) —U₁-arylene-arylene-aryl;
z) —U₁-alkylene-arylene-alkyl;
aa) -L₇-U₁-alkylene-aryl;
bb) -arylene-U₁-alkyl;
cc) -L₇-U₁-aryl;
dd) -L₇-U₁-heteroaryl;
ee) -L₇-U₁-cycloalkyl;
ff) -L₇-U₁-heterocyclyl;
gg) -L₇-U₁arylene-alkyl;
hh) -L₇-U₁-alkylene-arylene-alkyl;
ii) -L₇-U₁-alkyl;
jj) -L₇-U₁-alkylene-aryl-R₂₂;
kk) -L₇-U₁-alkylene-heteroaryl-R₂₂;
ll) -arylene-U₁-alkylene-R₂₂;
mm) -heteroarylene-U₁-alkylene-R₂₂;
nn) -L₇-U₁-aryl-R₂₂;
oo) -L₇-U₁-heteroarylene-R₂₂;
PP) -L₇-U₁-heteroaryl-R₂₂;
qq) -L₇-U₁-cycloalkyl-R₂₂;
rr) -L₇-U₁-heterocyclyl-R₂₂;
ss) -L₇-U₁-arylene-alkyl-R₂₂;
tt) -L₇-U₁heteroarylene-alkyl-R₂₂;
uu) -L₇-U₁-alkylene-arylene-alkyl-R₂₂;
vv) -L₇-U₁-alkylene-heteroarylene-alkyl-R₂₂;
ww) -L₇-U₁-alkylene-cycloalkylene-alkyl-R₂₂;
xx) -L₇-U₁-alkylene-heterocyclylene-alkyl-R₂₂;
yy) -L₇-U₁-alkylene-R₂₂;
zz) -L₇-U₁R₂₂;
aaa) -arylene-U₁—R₂₂;
bbb) -heteroarylene-U₁—R₂₂;
ccc) -heterocyclylene-U₁—R₂₂;
ddd) —U₁-alkylene-R₂₂;
eee) —U₁-arylene-R₂₂;
fff) —U₁-heteroarylene-R₂₂;
ggg) —U₁-alkylene-arylene-R₂₂;
hhh) —U₁-alkylene-heteroarylene-R₂₂;
iii) —U₁-heteroarylene-alkylene-R₂₂;
jjj) —U₁arylene-alkylene-R₂₂;
kkk) —U₁-cycloalkylene-alkylene-R₂₂;
lll) —U₁-heterocyclylene-alkylene-R₂₂;
mmm) —U₁-alkylene-arylene-alkyl-R₂₂;
nnn) —U₁-alkylene-heteroarylene-alkyl-R₂₂; 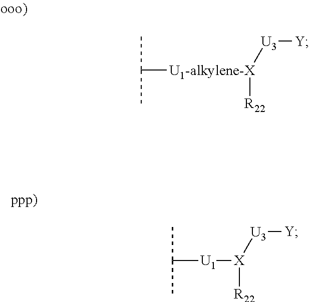
qqq) —U₁-alkylene-U₂-alkyl;
rrr) —U₁—U₂-alkyl; and
sss) -hydrogen
wherein
L₇ is a direct bond, -alkylene, -alkenylene, or -alkynylene;
U₁, U₂, and U₃ are independently selected from the group consisting of: a direct bond, —CH₂—, —O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, and —N(R₂₃)—N(R₂₄)—;
wherein
R₂₃ and R₂₄ are independently selected from the group consisting of: -hydrogen, —U₅-alkyl, —U₅-aryl, —U₅-perhaloalkyl, -arylene-alkyl, -alkylene-aryl, and -alkylene-arylene-alkyl; wherein U₅ is a direct bond, —SO₂—, —CO—, or —SO₂—NHCO₂—;
and when T is substituted with the group 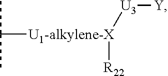
and U₁ is selected from the group consisting of —N(R₂₃)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —N(R₂₃)SO₂N(R₂₄)—, and —N(R₂₃)—N(R₂₄)—; then R₂₃ or R₂₄ may be fused with the alkylene group between U₁ and X to form a 5 to 7 membered ring;
X is 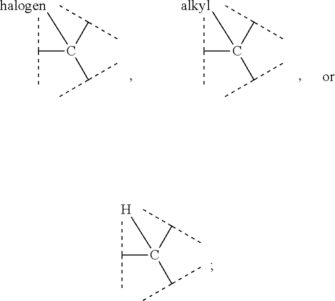
Y is hydrogen, -alkylene-aryl, -alkyl, -aryl, -heteroaryl, -heterocyclyl, -cycloalkyl, -alkylene-heteroaryl, or -alkylene-cycloalkyl;
R₂₂ is —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, acyloxy-alkylene-, or -alkylene-arylene-alkyl;
or a pharmaceutically acceptable salt, solvate, or prodrug thereof.

2. The compound of Formula (I) in claim 1 or a pharmaceutically acceptable salt or solvate thereof.

3. The compound of Formula (I) in claim 1 or a pharmaceutically acceptable salt or solvate thereof, wherein
R₂ is -L₃-D₁-arylene-D₂-G₁-G₂,
wherein
L₃ is a direct bond,
D₁ is a direct bond,
D₂ is a direct bond, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—,
wherein
R₅ and R₆ are independently selected from the group consisting of: -hydrogen, -alkyl, -aryl, and -alkylene-aryl,
G₁ is a direct bond or alkylene, and
G₂ is —CO₂H, —CO₂-alkyl, or an acid isostere, and
wherein
the arylene group of R₂ is optionally substituted with
a) -halo,
b) lower alkoxy optionally substituted 1 to 5 times with halo, or
c) -alkyl optionally substituted 1 to 5 times with halo.

4. The compound of Formula (I) in claim 3 or a pharmaceutically acceptable salt or solvate thereof, wherein W is —N(R₂)—, wherein
R₂ is -L₃-D₁-phenylene-D₂-G₁-G₂,
wherein
L₃ is a direct bond,
D₁ is a direct bond,
and either
D₂ is a direct bond,
G₁ is a direct bond, and
G₂ is selected from the group consisting of: —CO₂H, —CO₂-alkyl, and -acid isostere,
or
D₂ is —NH—,
G₁ is —CH₂—, and
G₂ is —CO₂H,
wherein
the phenylene group of R₂ is further optionally substituted with a substituent selected from the group consisting of: -halo, -perhaloalkyl, and —NHSO₂CH₃.

5. The compound of Formula (I) in claim 1 or a pharmaceutically acceptable salt or solvate thereof, wherein R₁ is hydrogen.

6. The compound of Formula (I) in claim 1 or a pharmaceutically acceptable salt or solvate thereof, wherein L₁ is

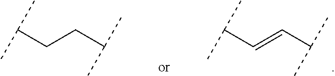

7. The compound of Formula (I) in claim 1 or a pharmaceutically acceptable salt or solvate thereof, wherein L₁ is

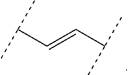

8. The compound of Formula (I) in claim 1 or a pharmaceutically acceptable salt or solvate thereof, wherein L₁ is —CH₂—.

9. The compound of Formula (I) in claim 1 or a pharmaceutically acceptable salt or solvate thereof, wherein Ar₁ is a phenyl group substituted 1 to 5 times, wherein the substituent(s) are independently selected from the group consisting of: -chloro and -fluoro.

10. The compound of Formula (I) in claim 1 or a pharmaceutically acceptable salt or solvate thereof, wherein Ar₂ is a phenylene or naphthylene group optionally having 1 to 5 substituents.

11. The compound of Formula (I) in claim 1 or a pharmaceutically acceptable salt or solvate thereof, wherein Ar₂ is a phenylene group or naphthylene group, wherein Ar₂ is substituted 1 to 5 times, wherein the substituent(s) are independently selected from the group consisting of:
a) -fluoro;
b) -chloro;
c) -bromo;
d) -iodo;
e) -Q-R₁₇;
f) -alkyl;
g) -aryl;
h) -arylene-alkyl;
i) -Q-alkyl; and
j) -arylene-Q-alkyl;
wherein
Q is —CH₂—, —O—, —C(O)—, or —C(O)—O—, and
R₁₇ is: -hydrogen, -alkyl, -aryl, —CO₂H, or an acid isostere.

12. The compound of Formula (I) in claim 1 or a pharmaceutically acceptable salt or solvate thereof, wherein Ar₂ is a phenylene group substituted 1 to 5 times, wherein the substituent(s) are independently selected from the group consisting of:
a) -fluoro;
b) -chloro;
c) -bromo;
d) -iodo;
e) -Q-R₁₇;
f) -alkyl;
g) -phenyl;
h) -phenylene-alkyl;
i) -Q-alkyl; and
j) -phenylene-Q-alkyl;
wherein
Q is —CH₂—, —O—, —C(O)—, or —C(O)—O—, and
R₁₇ is: -hydrogen, -alkyl, -phenyl, or —CO₂H.

13. The compound of Formula (I) in claim 1 or a pharmaceutically acceptable salt or solvate thereof, wherein L₂ is: —O—, —O-alkylene-, -alkylene-O, or a direct bond.

14. The compound of Formula (I) in claim 1 or a pharmaceutically acceptable salt or solvate thereof, wherein L₂ is: —O-alkylene- or a direct bond.

15. The compound of Formula (I) in claim 1 or a pharmaceutically acceptable salt or solvate thereof, wherein L₂ is —K—, wherein K is —O—, —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂—, —SO₂N(R₂₀)—, —C(O)—O—, —O—C(O)—, —S—, —S(O)—, —S(O₂)—, —N(R₂₀)SO₂N(R₂₁)—, —N═N—, or —N(R₂₀)—N(R₂₁)—.

16. The compound of Formula (I) in claim 1 or a pharmaceutically acceptable salt or solvate thereof, wherein L₂ is —K—, wherein K is —N(R₂₀)CO—, wherein R₂₀ is hydrogen or alkyl.

17. The compound of Formula (I) in claim 1 or a pharmaceutically acceptable salt or solvate thereof, wherein T is an aryl group optionally having 1 to 5 substituents.

18. The compound of Formula (I) in claim 1 or a pharmaceutically acceptable salt or solvate thereof, wherein T is an aryl group substituted by the group —U₁-alkylene-R₂₂, wherein U₁ is —O— or a direct bond, and R₂₂ is —CO₂H or an acid isostere.

19. The compound of Formula (I) in claim 1 or a pharmaceutically acceptable salt or solvate thereof, wherein Ar₂ is phenylene, L₂ is a direct bond, and T is phenyl such that —Ar₂-L₂-T taken together are a biphenyl group, wherein T is substituted with at least one group selected from the group consisting of: —U₁-alkyl, —U₁-perhaloalkyl, —U₁—R₂₂, fluoro, and chloro,
wherein
U₁ is a direct bond, —CO₂—, —O—, —S—, —NHSO₂—, —N(R₂₃)SO₂—, —SO₂—, —NHCO—, —NHCO₂—, or —NHCO₂NH—, wherein R₂₃ is —U₅-alkyl, wherein
U₅ is a direct bond or —SO₂—,
R₂₂ is alkyl, —CO₂H or acid isostere, and
wherein the alkyl group in —U₁-alkyl is optionally substituted 1 to 5 times with halo.

20. The compound of Formula (I) in claim 1 or a pharmaceutically acceptable salt or solvate thereof, wherein Ar₁ is 2,4-dichlorophenyl.

21. The compound of Formula (I) in claim 1 or a pharmaceutically acceptable salt or solvate thereof, wherein
Ar₂ is phenylene,
L₂ is a direct bond, —K— or -arylene-K—;
wherein K is —NH—SO₂—, —N(alkyl)-SO₂—, or —O—
T is phenyl substituted with at least one group selected from the group consisting of:
a) -fluoro;
b) -chloro;
c) -cyano;
d) -perfluoroalkyl;
e) —U₁-perfluoroalkyl;
f) —U₁-alkylene-R₂₂;
g) —U₁—R₂₂; and
e) -alkyl substituted 1 to 5 times with halo;
wherein
U₁ is —O—, direct bond, —SO₂—, or —NHSO₂—; and
R₂₂ is -alkyl, —SO₃H, —P(O)(OH)₂, —P(O)(O-alkyl)(OH), —CO₂H, —CO₂-alkyl, or an acid isostere.

22. The compound of Formula (I) in claim 1 or a pharmaceutically acceptable salt or solvate thereof, wherein
Ar₂ is phenylene,
L₂ is a direct bond,
T is thiophenyl substituted with at least one substituent selected from the group consisting of:
a) -fluoro,
b)-chloro,
c) -bromo,
d) -iodo,
e) -alkyl;
f) -alkyl substituted 1 to 5 times with halo; and
g) —U₁—R₂₂;
wherein
U₁ is —O—, direct bond, —SO₂—, or —NHSO₂—; and
R₂₂ is -alkyl, —SO₃H, —P(O)(OH)₂, —P(O)(O-alkyl)(OH), —CO₂H, —CO₂-alkyl, or an acid isostere.

23. The compound of Formula (I) in claim 1 or a pharmaceutically acceptable salt or solvate thereof, wherein
a and b are equal to zero, and
-L₁-Ar₂-L₂-T taken together are a group selected from the group consisting of:
2-[alkyl-benzenesulfonylamino-phenyl]-(E)-vinyl,
2-[(alkyl-benzylamino)-phenyl]-(E)-vinyl,
2-[(trifluoroalkyl-benzenesulfonylamino)-phenyl]-(E)-vinyl,
2-{[(alkyl-benzenesulfonyl)-alkyl-amino]-phenyl}-(E)-vinyl,
2-(4′-trifluoroalkoxy-biphenyl-4-yl)-(E)-vinyl,
2-(3′-trifluoroalkylsulfonyl amino-biphenyl-4-yl)-(E)-vinyl,
2-(3′-carboxy-biphenyl-4-yl)-(E)-vinyl,
2-(4′-carboxy-biphenyl-4-yl)-(E)-vinyl,
2-(3′-alkylsulfonyl-biphenyl-4-yl)-(E)-vinyl,
2-[4-(5-Chloro-thiophen-2-yl)-phenyl]-(E)-vinyl,
2-(4′-alkylsulfanyl-biphenyl-4-yl)-(E)-vinyl,
2-[(4-pyrimidin-3-yl)-phenyl]-(E)-vinyl,
2-[4-(5-acetyl-thiophen-2-yl-phenyl)]-(E)-vinyl,
2-[3′-(1,1,4-trioxo-1-[1,2,5]-thiadiazolidin-2-yl)-biphenyl-4-yl]-(E)-vinyl,
2-(4′-alkoxyoxycarbonylamino-3′-alkoxyoxy-biphenyl-4-yl)-(E)-vinyl,
2-(4′-amino-3′-alkoxy-biphenyl-4-yl)-(E)-vinyl,
2-[4′-(3-isopropyl-ureido)-3′-alkoxyoxy-biphenyl-4-yl]-(E)-vinyl, and
2-[4-(trifluoroalkyl-phenoxy)-phenyl]-(E)-vinyl.

24. The compound of Formula (I) in claim 1 or a pharmaceutically acceptable salt or solvate thereof, wherein
a and b are equal to zero, and
-L₁-Ar₂-L₂-T taken together are a group selected from the group consisting of:
3′-trifluoroalkyl-biphenyl-4-yl-methyl,
4′-trifluoroalkyl-biphenyl-4-yl-methyl,
(3′-alkylsulfonylamino-biphenyl-4-yl)-methyl, and
(4′-alkylsulfonylamino-biphenyl-4-yl)-methyl.

25. A pharmaceutically acceptable salt, solvate, or prodrug of a compound of Formula (I) according to claim 1.

26. A prodrug of a compound of Formula (I) of claim 25, wherein the prodrug comprises at least one of a biohydrolyzable ester or biohydrolyzable amide of a compound of Formula (I).

27. The compound of Formula (I) in claim 1 or a pharmaceutically acceptable salt or solvate thereof, wherein the alkyl and aryl groups in Ar₁, Ar₂, R₁ through R₄₄, and Y are optionally substituted 1 to 5 times with substituent(s) independently selected from the group consisting of:
a) -halogen;
b) -hydroxyl;
c) —U₄-alkyl; and
d) —U₄-alkylene-aryl;
wherein U₄ is selected from the group consisting of: —CH₂—, —O—, —N(H)—, —S—, —SO₂—, —CON(H)—, —NHC(O)—, —NHSO₂—, —SO₂N(H)—, —CO₂—, and —O—CO—.

28. A compound selected from the group consisting of:
1) 4-{4-(2,4-dichloro-phenyl)-2-[3-(3′-trifluoromethyl-biphenyl-4-yl)-propyl]-imidazol-1-yl}-benzoic acid;
2) 4-{4-(2,4-difluoro-phenyl)-2-[2-(3′-methanesulfonyl-biphenyl-4-yl)-(E)-vinyl]-imidazol-1-yl}-benzoic acid;
3) 4-{4-(3,4-dichloro-phenyl)-2-[2-(3′-methanesulfonyl-biphenyl-4-yl)-(E)-vinyl]-imidazol-1-yl}-benzoic acid;
4) 4-[4-(2,4-Dichloro-phenyl)-2-(4′-hydroxy-biphenyl-4-yl-methyl)-imidazol-1-yl]-benzoic acid;
5) 4-[4-(2,4-Dichloro-phenyl)-2-(3′-methanesulfonyl-biphenyl-4-yl-methyl)-imidazol-1-yl]-benzoic acid;
6) 4-[4-(2,4-Dichloro-phenyl)-2-(3′-trifluoromethyl-biphenyl-4-yl-methyl)-imidazol-1-yl]-benzoic acid;
7) 4-[4-(2,4-Dichloro-phenyl)-2-(4′-ethoxy-biphenyl-4-yl-methyl)-imidazol-1-yl]-benzoic acid;
8) 4-{4-(2,4-Dichloro-phenyl)-2-[4′-(propane-2-sulfonylamino)-biphenyl-4-yl-methyl]-imidazol-1-yl}-benzoic acid;
9) 4-{4-(2,4-Dichloro-phenyl)-2-[4′-(4,4,4-trifluoro-butoxy)-biphenyl-4-yl-methyl]-imidazol-1-yl}-benzoic acid;
10) 5-[4-(2,4-Dichloro-phenyl)-2-(4′-hydroxy-biphenyl-4-yl-methyl)-imidazol-1-yl]-2-trifluoromethyl-benzoic acid;
11) 4-[4-(2,4-Dichloro-phenyl)-2-(4′-hydroxy-biphenyl-4-yl-methyl)-imidazol-1-yl]-2-methanesulfonylamino-benzoic acid;
12) 4-[2-(4′-tert-Butyl-biphenyl-4-yl-methyl)-4-(2,4-dichloro-phenyl)-imidazol-1-yl]-2-methanesulfonylamino-benzoic acid;
13) 4-[4-(2,4-Dichloro-phenyl)-2-(4′-trifluoromethyl-biphenyl-4-yl-methyl)-imidazol-1-yl]-2-methanesulfonylamino-benzoic acid;
14) 2-amino-5-(4′-{2-[4-(2,4-dichloro-phenyl)-1-ethyl-1H-imidazol-2-yl]-(E)-vinyl}-biphenyl-4-yl-oxy)-benzoic acid;
15) 5-{4-[4-(2,4-dichloro-phenyl)-2-(3′-methanesulfonyl-biphenyl-4-yl-methyl)-imidazol-1-yl]-phenyl}-1,2,5-thiadiazolidine-3-one-1,1-dioxide;
16) 4-{4-(2,4-Dichloro-phenyl)-2-[2-(3′-methanesulfonyl-biphenyl-4-yl)-(E)-vinyl]-imidazol-1-yl}-benzoic acid;
17) 4-{4-(2,4-Dichloro-phenyl)-2-[2-(3′-trifluoromethyl-biphenyl-4-yl)-(E)-vinyl]-imidazol-1-yl}-benzoic acid;
18) 4-{4-(2,4-Dichloro-phenyl)-2-[2-(3′-methanesulfonylamino-biphenyl-4-yl)-(E)-vinyl]imidazol-1-yl}-benzoic acid;
19) 4-(4-(2,4-Dichloro-phenyl)-2-{2-[3′-(2,2,2-trifluoro-ethanesulfonylamino)-biphenyl-4-yl]-(E)-vinyl}-imidazol-1-yl)-benzoic acid;
20) 4-(4-(2,4-Dichloro-phenyl)-2-{2-[3′-(propane-2-sulfonylamino)-biphenyl-4-yl]-(E)-vinyl}-imidazol-1-yl)-benzoic acid;
21) 3-{4-(2,4-Dichloro-phenyl)-2-[2-(3′-methanesulfonyl-biphenyl-4-yl)-(E)-vinyl]-imidazol-1-yl}-benzoic acid;
22) 5-(4-{4-(2,4-dichloro-phenyl)-2-[2-(3′-methanesulfonyl-biphenyl-4-yl)-(E)-vinyl]-imidazol-1-yl}-phenyl)-1,2,5-thiadiazolidine-3-one-1,1-dioxide;
23) (±)-4-(4-{4-(2,4-dichloro-phenyl)-2-[2-(3′-methanesulfonyl-biphenyl-4-yl)-(E)-vinyl]-imidazol-1-yl}-phenyl)-1,1-dioxo-1,2,5-thiadiazolidin-3-ylideneamine;
and pharmaceutically acceptable salts or solvates thereof.

29. A pharmaceutical composition comprising a compound of Formula (I) as claimed in claim 1 or a pharmaceutically acceptable salt or solvate thereof, and a pharmaceutically acceptable carrier, excipient, or diluent.

30. A pharmaceutical composition comprising a compound of Formula (I) as claimed in claim 2 or a pharmaceutically acceptable salt or solvate thereof, and a pharmaceutically acceptable carrier, excipient, or diluent.

31. A pharmaceutical composition comprising a compound of Formula (I) as claimed in claim 3 or a pharmaceutically acceptable salt or solvate thereof, and a pharmaceutically acceptable carrier, excipient, or diluent.

32. A pharmaceutical composition comprising a compound of Formula (I) as claimed in claim 4 or a pharmaceutically acceptable salt or solvate thereof, and a pharmaceutically acceptable carrier, excipient, or diluent.

33. A pharmaceutical composition comprising a compound of Formula (I) as claimed in claim 5 or a pharmaceutically acceptable salt or solvate thereof, and a pharmaceutically acceptable carrier, excipient, or diluent.

34. A pharmaceutical composition comprising a compound of Formula (I) as claimed in claim 6 or a pharmaceutically acceptable salt or solvate thereof, and a pharmaceutically acceptable carrier, excipient, or diluent.

35. A pharmaceutical composition comprising a compound of Formula (I) as claimed in claim 7 or a pharmaceutically acceptable salt or solvate thereof, and a pharmaceutically acceptable carrier, excipient, or diluent.

36. A pharmaceutical composition comprising a compound of Formula (I) as claimed in claim 8 or a pharmaceutically acceptable salt or solvate thereof, and a pharmaceutically acceptable carrier, excipient, or diluent.

37. A pharmaceutical composition comprising a compound of Formula (I) as claimed in claim 9 or a pharmaceutically acceptable salt or solvate thereof, and a pharmaceutically acceptable carrier, excipient, or diluent.

38. A pharmaceutical composition comprising a compound of Formula (I) as claimed in claim 10 or a pharmaceutically acceptable salt or solvate thereof, and a pharmaceutically acceptable carrier, excipient, or diluent.

39. A pharmaceutical composition comprising a compound of Formula (I) as claimed in claim 11 or a pharmaceutically acceptable salt or solvate thereof, and a pharmaceutically acceptable carrier, excipient, or diluent.

40. A pharmaceutical composition comprising a compound of Formula (I) as claimed in claim 12 or a pharmaceutically acceptable salt or solvate thereof, and a pharmaceutically acceptable carrier, excipient, or diluent.

41. A pharmaceutical composition comprising a compound of Formula (I) as claimed in claim 13 or a pharmaceutically acceptable salt or solvate thereof, and a pharmaceutically acceptable carrier, excipient, or diluent.

42. A pharmaceutical composition comprising a compound of Formula (I) as claimed in claim 14 or a pharmaceutically acceptable salt or solvate thereof, and a pharmaceutically acceptable carrier, excipient, or diluent.

43. A pharmaceutical composition comprising a compound of Formula (I) as claimed in claim 15 or a pharmaceutically acceptable salt or solvate thereof, and a pharmaceutically acceptable carrier, excipient, or diluent.

44. A pharmaceutical composition comprising a compound of Formula (I) as claimed in claim 16 or a pharmaceutically acceptable salt or solvate thereof, and a pharmaceutically acceptable carrier, excipient, or diluent.

45. A pharmaceutical composition comprising a compound of Formula (I) as claimed in claim 17 or a pharmaceutically acceptable salt or solvate thereof, and a pharmaceutically acceptable carrier, excipient, or diluent.

46. A pharmaceutical composition comprising a compound of Formula (I) as claimed in claim 18 or a pharmaceutically acceptable salt or solvate thereof, and a pharmaceutically acceptable carrier, excipient, or diluent.

47. A pharmaceutical composition comprising a compound of Formula (I) as claimed in claim 19 or a pharmaceutically acceptable salt or solvate thereof, and a pharmaceutically acceptable carrier, excipient, or diluent.

48. A pharmaceutical composition comprising a compound of Formula (I) as claimed in claim 20 or a pharmaceutically acceptable salt or solvate thereof, and a pharmaceutically acceptable carrier, excipient, or diluent.

49. A pharmaceutical composition comprising a compound of Formula (I) as claimed in claim 21 or a pharmaceutically acceptable salt or solvate thereof, and a pharmaceutically acceptable carrier, excipient, or diluent.

50. A pharmaceutical composition comprising a compound of Formula (I) as claimed in claim 22 or a pharmaceutically acceptable salt or solvate thereof, and a pharmaceutically acceptable carrier, excipient, or diluent.

51. A pharmaceutical composition comprising a compound of Formula (I) as claimed in claim 23 or a pharmaceutically acceptable salt or solvate thereof, and a pharmaceutically acceptable carrier, excipient, or diluent.

52. A pharmaceutical composition comprising a compound of Formula (I) as claimed in claim 24 or a pharmaceutically acceptable salt or solvate thereof, and a pharmaceutically acceptable carrier, excipient, or diluent.

53. A pharmaceutical composition comprising a compound as claimed in claim 28 or a pharmaceutically acceptable salt or solvate thereof, and a pharmaceutically acceptable carrier, excipient, or diluent.


SEQUENCE LISTING (1 sequence)

SEQ ID NO: 1 - protein, 9 aa, Artificial Sequence
  Synthetic
ENDXINASL